(19) United States
(12) Patent Application Publication
(10) Pub. No.: US 2010/0037342 A1
(43) Pub. Date: Feb. 11, 2010

(54) METHODS AND COMPOSITIONS FOR BREEDING PLANTS WITH ENHANCED YIELD

(75) Inventors: Glenn Richard Johnson, Urbana, IL (US); Xiaofeng S. Yang, Chesterfield, MO (US)

Correspondence Address: MONSANTO COMPANY, 800 N. LINDBERGH BLVD., ATTENTION: GAIL P. WUELLNER, IP PARALEGAL, (E1NA), ST. LOUIS, MO, 63167, US

(73) Assignee: Monsanto Technology LLC, St. Louis, MO (US)

(21) Appl. No.: 12/533,136

(22) Filed: Jul. 31, 2009

Related U.S. Application Data
(60) Provisional application No. 61/085,501, filed on Aug. 1, 2008.

(51) Int. Cl. A01H 1/00

(52) U.S. Cl. 800/263; 800/260; 800/264; 800/265; 800/266

(57) ABSTRACT

The present invention provides breeding methods and compositions to enhance the germplasm of a plant by the use of direct nucleic acid sequence information. The methods describe the identification and accumulation of preferred nucleic acid sequences in the germplasm of a breeding population of crop plants.

### CROSS-REFERENCE TO RELATED APPLICATIONS

This application claims priority to U.S. Provisional Application No. 61/085,501 (filed Aug. 1, 2008), which is incorporated by reference herein in its entirety.

### FIELD OF INVENTION

This invention is in the field of plant breeding. More specifically, this invention relates to the use of expression profiling technology in activities related to germplasm improvement and to methods and compositions for breeding crop plants with enhanced yield.

### INCORPORATION OF SEQUENCE LISTING

A sequence listing containing the file named “54066seq.txt” which is 422 bytes (measured in MS-Windows®) and created on Jul. 28, 2008, comprises 438 nucleotide sequences, and is herein incorporated by reference in its entirety.

### BACKGROUND OF INVENTION

A key objective of plant breeding is to increase genetic gain, often in terms of yield. Plant breeders have historically relied on phenotypic measurements as the basis of selection in order to achieve yield gain. There remains a need in the art for the identification of methods and compositions to increase the efficiency of plant breeding.

The present invention utilizes expression profiling technology in combination with a modified association study approach to map and identify targets for yield in corn plants. The compositions provided herein are useful in marker-assisted selection and as expression constructs in transgenic corn plants. The methods provided herein are exemplified in corn plants though the inventors contemplate utility in other crop plants, as well as in traits including but not limited to yield.

### SUMMARY OF INVENTION

In one embodiment, the present invention is directed to a method of plant breeding. The method comprises determining the expression profile for at least one nucleic acid within the genome of at least one crop plant in a breeding population; associating the determined expression profile with at least one numerical value wherein the numerical value is related to one or more phenotypic traits; and making a plant breeding decision for the one or more crop plants based on the association. In more particular embodiments, the step of making the plant breeding decision may comprise selecting among breeding populations based on the at least one numerical value; selecting progeny in one or more breeding populations based on the at least one numerical value;

predicting progeny performance of parental lines and selecting parental lines based on the predicted progeny performance; advancing lines in germplasm improvement activities based on the at least one numerical value; and selecting for at least one phenotypic trait based on the at least one numerical value associated with another phenotypic trait.

In another embodiment, the invention is directed to a method of plant breeding comprising providing at least two plants wherein at least one expression profile is assayed for at least one locus for at each plant; determining an expression profile effect estimate for at least one phenotypic trait for at least two expression profiles for the at least one locus; and making plant breeding decisions based on the determined expression profile effect estimate for the at least one phenotypic trait.

In another embodiment, the invention is directed to a method of plant breeding comprising establishing a fingerprint map defining a plurality of loci within the genome of a breeding population; associating a QTL allele with known map location with a phenotypic trait; and assaying at least one other plant for presence of the QTL allele using at least one marker corresponding to at least one of the plurality of expression profiles to predict expression of the phenotypic trait.

In another embodiment, the invention is directed to a method of marker assisted breeding comprising providing a breeding population comprising at least two plants; associating at least one phenotypic trait with at least one locus of the genome of the plants of the breeding population, wherein the locus is defined by at least one nucleic acid sequence; and assaying for the presence of the at least one nucleic acid sequence of the at least one locus to predict expression of at least one phenotypic trait in a progeny plant of the breeding population.

In another embodiment, the invention is directed to a method of selecting a breeding population for use in a breeding program. The method comprises providing at least two distinct breeding populations; using a plurality of breeding values for at least one phenotypic trait for at least two expression profiles for at least one locus for the breeding populations; and selecting at least one breeding population based on at least one breeding value.

In a further embodiment, the invention is directed to an isolated nucleic acid marker comprising at least 20 consecutive nucleotides corresponding to a nucleic acid designated as SEQ ID NO 1-438, associated with enhanced yield.

In another embodiment, the invention is directed to a plant or parts thereof comprising at least one nucleic acid at least 80% identical to a nucleic acid selected from the group consisting of SEQ ID NO 1-438.

In another embodiment, the invention is directed to a substantially purified nucleic acid molecule comprising a nucleic acid sequence wherein said nucleic acid sequence exhibits 95% or greater identity to a nucleic acid sequence selected from the group consisting of SEQ ID NO: 1 through SEQ ID NO: 338 and sequences complementary to SEQ ID NO: 1 through SEQ ID NO: 338.

In still a further embodiment, the invention is directed to a transgenic plant comprising a nucleic acid molecule, wherein the nucleic acid molecule comprises: (a) an exogenous promoter region that functions in a plant cell to cause the production of an mRNA molecule, operably linked to (b) a structural nucleic acid molecule comprising a nucleic acid sequence having 95% or greater identity to a nucleic acid sequence selected from the group consisting of: (i) SEQ ID NO: 1 through SEQ ID NO: 338, (ii) a sequence complementary to SEQ ID NO: 1 through SEQ ID NO: 338, and (iii) any fragment of a nucleic acid sequence from “(i)” and/or “(ii)”; operably linked to (c) a 3′ non-translated sequence that functions in said plant cell to cause termination of transcription and/or addition of polyadenylated ribonucleotides to the 3′ end of said mRNA molecule.

The present invention includes a method for breeding of a crop plant, such as maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, oilseed, and root crops, wherein oilseed crops include soybean, canola, oil seed rape, oil palm, sunflower, olive, corn, cottonseed, peanut, flaxseed, safflower, and coconut, with enhanced traits comprising at least one sequence of interest, further defined as conferring a preferred property selected from the group consisting of herbicide tolerance, disease resistance, insect or pest resistance, altered fatty acid, protein or carbohydrate metabolism, increased grain yield, increased oil, increased nutritional content, increased growth rates, enhanced stress tolerance, preferred maturity, enhanced organoleptic properties, altered morphological characteristics, other phenotypic traits, traits for industrial uses, or traits for improved consumer appeal, wherein said traits may be nontransgenic or transgenic.

Non-limiting examples of silage quality traits include brown midrib (BMR) traits, in vitro digestibility of dry matter, leafiness, horny endosperm, crude protein, neutral detergent fiber, neutral detergent fiber digestibility, starch content, starch availability, kernel texture, milk/ton, fat content of milk, readily available energy, soluble carbohydrate digestibility, nonsoluble carbohydrate digestibility, reduced phytate production, reduced waste production, and silage yield.

Non-limiting examples of grain quality traits for biofuel yield include total biomass, fermentation yield, fermentation kinetics, total starch, extractable starch, starch morphology, phosphorous availability, waxy traits, glucose content, total oil content, germ oil content, endosperm oil content, fatty acid composition, kernel or seed morphology, amylose content, amylopectin content, protein composition and content (in particular, for end-use in animal feed following fractionation).

The present invention also provides for plants and parts thereof with compositions of one or more preferred nucleic acid sequences as described herein.

### DETAILED DESCRIPTION OF THE INVENTION

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts et al., Molecular Biology of The Cell, 5^{th} Edition, Garland Science Publishing, Inc.: New York, 2007; Rieger et al., Glossary of Genetics: Classical and Molecular, 5^{th} edition, Springer-Verlag: New York, 1991; King et al, A Dictionary of Genetics, 6^{th} ed, Oxford University Press: New York, 2002; and Lewin, Genes IX, Oxford University Press: New York, 2007. The nomenclature for DNA bases as set forth at 37 CFR §1.822 is used.

An “allele” refers to an alternative sequence at a particular locus; the length of an allele can be as small as 1 nucleotide base, but is typically larger. Allelic sequence can be denoted as nucleic acid sequence or as amino acid sequence that is encoded by the nucleic acid sequence.

A “locus” is a position on a genomic sequence that is usually found by a point of reference; e.g., a short DNA sequence that is a gene, or part of a gene or intergenic region. The loci of this invention comprise one or more polymorphisms in a population; i.e., alternative alleles present in some individuals.

As used herein, an “expression variant” refers to cases where two or more distinct RNA variants are transcribed from a DNA sequence. Expression variants can comprise alternative ribonucleotide arrangements wherein one or more introns are retained or one or more exons are spliced out. Expression variants can result in variant phenotypes, i.e., distinct polypeptides.

As used herein, “expression profile” means the characterization of a transcription product of a nucleic acid. In one aspect, an expression profile is presented as the relative hybridization of at least one target nucleic acid with at least one test nucleic acid, wherein the hybridization level may indicate up-regulation, down-regulation, or no change in expression level.

As used herein, “expression profile assay” means a method to query at least one target nucleic acid with at least one test nucleic acid using any means available in the art, including future platforms designed for measuring relative hybridization between two or more pools of nucleic acids. Non-limiting examples of platforms for detection of expression levels include DNA microarrays, DNA arrays, DNA chips, gene chips, bead-based arrays, RT-PCR, quantitative-RT PCR, and Northern blots.

As used herein, “target nucleic acid” means at least one nucleic acid physically associated with a substrate such as a bead, glass slide, or other microarray platform. The target nucleic acid may comprise an oligonucleotide sequence, a partial gene sequence, a full gene sequence, a non-coding sequence, and may overlap in sequence with other target nucleic acids.

As used herein, “test nucleic acid” means at least one nucleic acid that is combined with the target nucleic acid in order to evaluate level of hybridization, i.e., level of complementarity. This allows for the identification of polymorphisms between the test and target sequence, inference of regulatory modules, comparison of at least two test nucleic acids, and, in conjunction with phenotype data, association of expression profile levels with trait values to map QTL associated with expression (eQTL) or hybridization level (hQTL). In certain aspect, a test nucleic acid may RNA or cDNA.

Further, any nucleic acid, as used herein, may comprise one or more haplotypes, portions of one or more haplotypes, one or more genes, portions of one or more genes, one or more QTL, and portions of one or more QTL. In addition, a plurality of nucleic acids can comprise one or more haplotypes, portions of one or more haplotypes, one or more genes, portions of one or more genes, one or more QTL, and portions of one or more QTL. The nucleic acid may originate from a DNA or RNA template, either directly or indirectly (i.e., cDNA obtained from reverse transcription of mRNA).

As used herein, “expression profile effect estimate” means a predicted effect estimate for an expression profile reflecting association with one or more phenotypic traits, wherein said associations can be made de novo or by leveraging historical expression profile-trait association data or genotype-trait association data wherein at least one genotype is identified to be associated with at least one expression profile.

As used herein, “breeding value” means a calculation based on expression profile effect estimates and expression profile frequency values, wherein expression profile frequency can be measured in terms of the underlying nucleic acid sequence associated with the expression profile. The breeding value of a specific expression profile relative to other expression profiles at the same locus (i.e., haplotype window), or across loci (i.e., haplotype windows), can also be determined. In other words, the change in population mean by fixing a nucleic acid sequence associated with a particular expression profile is determined. In addition, in the context of evaluating the effect of substituting a specific region in the genome, either by introgression or a transgenic event, breeding values provide the basis for comparing specific nucleic acid sequences, and their corresponding expression profiles, for substitution effects. Also, in hybrid crops, the breeding value of expression profiles can be calculated in the context of the expression profile in the tester used to produce the hybrid.

As used herein, “genotype” means the genetic component of the phenotype and it can be indirectly characterized using markers or directly characterized by nucleic acid sequencing. Suitable markers include a phenotypic character, a metabolic profile, a genetic marker, or some other type of marker. A genotype may constitute an allele for at least one genetic marker locus or a haplotype for at least one haplotype window. In some embodiments, a genotype may represent a single locus and in others it may represent a genome-wide set of loci. In another embodiment, the genotype can reflect the sequence of a portion of a chromosome, an entire chromosome, a portion of the genome, and the entire genome. As opposed to a genetic marker such as a SNP, where the genotype comprises a single nucleotide, the genotype identified with the expression profiling may constitute a plurality of nucleotides, where the length of the genotype is contingent on the length of the target nucleic acid sequence and the number of nucleotides at any locus is contingent on the number of probes (i.e. bi-allelic vs. all four possible base pairs for DNA or RNA). Notably, a genetic marker assay as known in the art (e.g., SNP detection via TaqMan) detects only two alleles. An advantage of the present invention is the potential to directly query all four nucleotides (adenine, A; thymine, T; cytosine, C; and guanine, G) simultaneously at any one nucleotide position based on the representation of target nucleic acids in the expression profile assay. That is, for any one base pair position, there will be twice the information when using direct nucleic acid sequencing versus a genetic marker assays. This can be very important in determining whether two lines share DNA that is identical by decent. With a SNP genotype, one can only assess whether a pair of alternative nucleic acid bases exist at a single nucleotide locus. As used herein, an expression profile or a nucleic acid associated with an expression profile from each of two or more individual plants from the same genomic region, that may or may not be associated with one or more phenotypic trait values, provides the basis for decisions related to germplasm improvement activities, wherein one or more loci can be evaluated. Knowing whether two sequences at a locus are completely identical or if they contain combinations of identical and non-identical loci can aid in determining whether the loci have the same trait value, are linked to the same traits or are identical by descent. Therefore in another aspect, one or more nucleic acid sequences from one or more individual plants that are associated with a phenotypic trait value can provide the basis for decisions related to germplasm improvement activities.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found, or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs) a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms. In addition, the presence, absence, or variation in copy number of the preceding may comprise a polymorphism.

As used herein, the term “single nucleotide polymorphism,” also referred to by the abbreviation “SNP,” means a polymorphism at a single site wherein said polymorphism constitutes a single base pair change, an insertion of one or more base pairs, or a deletion of one or more base pairs.

As used herein, “marker” means a detectable characteristic that can be used to discriminate between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics. As used herein, “genetic marker” means polymorphic nucleic acid sequence or nucleic acid feature.

As used herein, “marker assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc.

As used herein, the term “haplotype” means a chromosomal region within a haplotype window. Typically, the unique marker fingerprint combinations in each haplotype window define and differentiate individual haplotypes for that window. As used herein, a haplotype is defined and differentiated by one or more nucleic acid sequences at one or more loci within a “haplotype window,” wherein a nucleic acid sequence may represent a single base pair genotype (i.e., a SNP), an insertion or deletion, one or more contiguous base pairs (i.e., sequence nucleic acid, or a corresponding expression profile.

As used herein, the term “haplotype window” means a chromosomal region that is established by statistical analyses known to those of skill in the art and is in linkage disequilibrium. In the art, identity by state between two inbred individuals (or two gametes) at one or more molecular marker loci located within this region is taken as evidence of identity-by-descent of the entire region, wherein each haplotype window includes at least one polymorphic molecular marker. As used herein, haplotype windows are defined by at least two polymorphisms for at least one locus. Haplotype windows can be mapped along each chromosome in the genome and do not necessarily need to be contiguous. Haplotype windows are not fixed per se and, given the ever-increasing amount of nucleic acid sequence information, this invention anticipates the number and size of haplotype windows to evolve, with the number of windows increasing and their respective sizes decreasing, thus resulting in an ever-increasing degree confidence in ascertaining identity by descent based on the identity by state of genotypes. The advantage of using haplotype windows to delineate genomic regions of interest is the fact these genomic regions tend to be inherited as linkage blocks and thus are informative for association mapping and for tracking across multiple generations.

As used herein, “phenotype” means the detectable characteristics of a cell or organism which can be influenced by gene expression.

As used herein, “consensus sequence” means a constructed DNA sequence which identifies single nucleotide and indel polymorphisms in alleles at a locus. Consensus sequence can be based on either strand of DNA at the locus and states the nucleotide base of either one of each SNP in the locus and the nucleotide bases of all Indels in the locus. Thus, although a consensus sequence may not be a copy of an actual DNA sequence, a consensus sequence is useful for precisely designing primers and probes for actual polymorphisms in the locus.

As used herein, “linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

As used herein, “linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium. Two loci are said to be “genetically linked” when they are in linkage disequilibrium.

As used herein, “quantitative trait locus (QTL)” means a locus that controls to some degree numerically representable traits that are usually continuously distributed.

As used herein, “nucleic acid sequencing” means the determination of the order of nucleotides in a sample of nucleic acids, wherein nucleic acids include DNA and RNA molecules. “High throughput nucleic acid sequencing” means an automated and massively parallel approach for the determination of nucleotides in a sample of nucleic acids wherein examples of high throughput nucleic acid sequencing technology include, but are not limited to, platforms provided by 454 Life Sciences, Agencourt Bioscience, Applied Biosystems, LI-COR Biosciences, Microchip Biotechnologies, Network Biosystems, NimbleGen Systems, Illumina (Solexa), and VisiGen Biotechnologies, comprising but not limited to formats such as parallel bead arrays, sequencing by synthesis, sequencing by ligation, capillary electrophoresis, electronic microchips, “biochips,” microarrays, parallel microchips, and single-molecule arrays, as reviewed by Service (Science 2006 311:1544-1546).

As used herein, “aligning” or “alignment” of two or more nucleic acid sequences is the comparison of the nucleic acid sequences found at the same locus. Several methods of alignment are known in the art and are included in most of the popular bioinformatics packages.

As used herein, the term “primer” means a single strand of synthetic oligonucleotide, preferably from 10 to 120 nucleotides, which can be synthesized chemically or assembled from several chemically synthesized oligonucleotides. As used herein, primers may be used to initiate sequencing reactions and polymerase reactions, such as in gap fill reactions and PCR. As used herein, a primer will hybridize under the assay conditions specifically to a desired target sequence. As used herein, primers may be used to introduce a DNA tag, to introduce chemically modified bases, such as biotin labeled bases, or to introduce a hybridization sequence that can subsequently be used for capture, such as capture to a sequencing matrix or to an avidin-containing surface.

As used herein, “DNA amplification” means the in vitro synthesis of double stranded DNA through the use of a DNA polymerase. Typically, this is accomplished in a polymerase chain reaction (PCR) assay but may also include other methods such as a gap-fill reaction, mis-match repair, Klenow reaction, etc. DNA amplification is used to provide detectable or excess amounts of a specific DNA. It can also be used to incorporate into a target nucleic acid, hybridized probes, annealed adaptors and primers which may include specific functionality or information.

As used herein, the term “biomarker” refers to a marker representing at least one phenotype of interest in an organism, such as a crop plant, wherein the marker constitutes the characterization of a nucleic acid expression level or protein level such that is it present or absent, elevated, decreased, temporally variable, spatially variable, or quantifiable in some other aspect.

As used herein, the term “corn” means Zea mays or maize and includes all plant varieties that can be bred with corn, including wild maize species.

As used herein, the term “soybean” means Glycine max and includes all plant varieties that can be bred with soybean, including wild soybean species.

As used herein, the term “canola” means Brassica napus and B. campestris and includes all plant varieties than can be bred with canola, including wild Brassica species and other agricultural Brassica species.

As used herein, the term “elite line” means any line that has resulted from breeding and selection for superior agronomic performance. An elite plant is any plant from an elite line.

In accordance with the present invention, Applicants have discovered methods for breeding plants wherein breeding decisions can be made genotypically on the expression profile of at least one nucleic acid sequence associated with at least one phenotypic trait of interest. For example, the methods of the present invention provide for use of expression profiling technology to identify targets for selection and to serve as a secondary marker for making selection decisions for at least one locus of interest. Further, the methods of the present invention allow for improved flexibility, wherein the entire genome can be queried without reliance on pre-determined genetic markers and the development of genetic marker detection assays. In addition, any length of sequence from any locus can be leveraged to 1) determine genotype-trait associations, 2) discriminate between two or more lines, 3) predict line performance or hybrid performance and, ultimately, 4) provide the basis for improved breeding activities related to germplasm improvement. Further, this invention provides compositions for increased yield in a crop plant, specifically maize. This invention also provides methods and compositions for use of expression profiling technology as a biomarker to screen plants for response to stress or biological processes.

### A. Marker-Assisted Breeding

Breeding has advanced from selection for economically important traits in plants and animals based on phenotypic records of an individual and its relatives to the application of molecular genetics to identify genomic regions that contain valuable genetic traits. Inclusion of genetic markers in breeding programs has accelerated the genetic accumulation of valuable traits into a germplasm compared to that achieved based on phenotypic data only. Herein, “germplasm” includes breeding germplasm, breeding populations, collection of elite inbred lines, populations of random mating individuals, and biparental crosses. Genetic marker alleles (an “allele” is an alternative sequence at a locus) are used to identify plants that contain a desired genotype at multiple loci, and that are expected to transfer the desired genotype, along with a desired phenotype to their progeny. Genetic marker alleles can be used to identify plants that contain the desired genotype at one marker locus, several loci, or a haplotype, and that would be expected to transfer the desired genotype, along with a desired phenotype to their progeny. This process has been widely referenced and has served to greatly economize plant breeding by accelerating the fixation of advantageous alleles and also eliminating the need for phenotyping every generation.

Molecular breeding is often referred to as marker-assisted selection (MAS) and marker-assisted breeding (MAB), wherein MAS refers to making breeding decisions on the basis of molecular marker genotypes and MAB is a general term representing the use of molecular markers in plant breeding. In these types of molecular breeding programs, genetic marker alleles can be used to identify plants that contain the desired genotype at one marker locus, several loci, or a haplotype, and that would be expected to transfer the desired genotype, along with a desired phenotype to their progeny. Markers are highly useful in plant breeding because once established, they are not subject to environmental or epistatic interactions. Furthermore, certain types of markers are suited for high throughput detection, enabling rapid identification in a cost effective manner.

Marker discovery and development in crops provides the initial framework for applications to MAB (U.S. Pat. No. 5,437,697; US Patent Applications 2005/0204780, 2005/0216545, 2005/0218305). The resulting “genetic map” is the representation of the relative position of characterized loci (DNA markers or any other locus for which alleles can be identified) along the chromosomes. The measure of distance on this map is relative to the frequency of crossover events between sister chromatids at meiosis. As a set, polyallelic markers have served as a useful tool for fingerprinting plants to inform the degree of identity of lines or varieties (U.S. Pat. No. 6,207,367). These markers form the basis for determining associations with phenotype and can be used to drive genetic gain. The implementation of MAS, wherein selection decisions are based on marker genotypes, is dependent on the ability to detect underlying genetic differences between individuals.

Many individuals and companies have developed their own proprietary and trade marked versions of molecular breeding. One aspect in common is that they are based on markers to report differences which are then used to make selections. However, these markers provide no or very limited information on the differences at the DNA sequence level; for example, a typical biallelic SNP marker provides information on only one base pair position and it can only distinguish between 2, rather than 4, nucleotides. Using expression profile assays gives the power to query 4 nucleotides at any given position within a nucleic acid sequence as directed by inclusion of target nucleic acid sequences. Furthermore, this power will be useful to fingerprint plant populations or lineages to allow genome wide discovery of useful variation, build pedigrees or calculate breeding values.

### 1. Marker Technologies

The development of markers and the association of markers with phenotypes, or quantitative trait loci (QTL) mapping for marker-assisted breeding has advanced in recent years. Examples of genetic markers are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), and others known to those skilled in the art. Marker discovery and development in crops provides the initial framework for applications to marker-assisted breeding activities (US Patent Applications 2005/0204780, 2005/0216545, 2005/0218305, and 2006/00504538). The resulting “genetic map” is the representation of the relative position of characterized loci (DNA markers or any other locus for which alleles can be identified) along the chromosomes. The measure of distance on this map is relative to the frequency of crossover events between sister chromatids at meiosis.

As a set, polymorphic markers serve as a useful tool for fingerprinting plants to inform the degree of identity of lines or varieties (U.S. Pat. No. 6,207,367). These markers form the basis for determining associations with phenotype and can be used to drive genetic gain. The implementation of marker-assisted selection is dependent on the ability to detect underlying genetic differences between individuals.

Genetic markers for use in the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

Nucleic acid molecules or fragments thereof are capable of specifically hybridizing to other nucleic acid molecules under certain circumstances. As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, molecules are exhibit “complete complementarity” when every nucleotide of one of the molecules is complementary to a nucleotide of the other. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al., In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The nucleic-acid probes and primers of the present invention can hybridize under stringent conditions to a target DNA sequence. The term “stringent hybridization conditions” is defined as conditions under which a probe or primer hybridizes specifically with a target sequence(s) and not with non-target sequences, as can be determined empirically. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa 1984 Nucl. Acids Res. 12:203-213; and Wetmur et al. 1968 J. Mol. Biol. 31:349-370. Appropriate stringency conditions that promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y., 1989, 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

A fragment of a nucleic acid molecule can be any sized fragment and illustrative fragments include fragments of nucleic acid sequences set forth in SEQ ID NO: 1-176 and complements thereof. In one aspect, a fragment can be between 15 and 25, 15 and 30, 15 and 40, 15 and 50, 15 and 100, 20 and 25, 20 and 30, 20 and 40, 20 and 50, 20 and 100, 25 and 30, 25 and 40, 25 and 50, 25 and 100, 30 and 40, 30 and 50, and 30 and 100. In another aspect, the fragment can be greater than 10, 15, 20, 25, 30, 35, 40, 50, 100, or 250 nucleotides.

Additional genetic markers can be used to select plants with an allele of a QTL associated with transgene modulating loci of the present invention. Examples of public marker databases include, for example: Maize Genome Database, Agricultural Research Service, United States Department of Agriculture or Soybase, an Agricultural Research Service, United States Department of Agriculture.

In another embodiment, markers, such as single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with alleles of a QTL of the present invention can be utilized.

In one embodiment, nucleic acid-based analyses for the presence or absence of the genetic polymorphism can be used for the selection of seeds in a breeding population. A wide variety of genetic markers for the analysis of genetic polymorphisms are available and known to those of skill in the art. The analysis may be used to select for genes, portions of genes, QTL, alleles, or genomic regions (haplotypes) that comprise or are linked to a genetic marker.

Herein, nucleic acid analysis methods are known in the art and include, but are not limited to, PCR-based detection methods (for example, TaqMan assays), microarray methods, and nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form.

Polymorphisms in DNA sequences can be detected or typed by a variety of effective methods well known in the art including, but not limited to, those disclosed in U.S. Pat. Nos. 5,468,613 and 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464, 7,312,039, 7,238,476, 7,297,485, 7,282,355, 7,270,981, and 7,250,252 all of which are incorporated herein by reference in their entireties. However, the compositions and methods of this invention can be used in conjunction with any polymorphism typing method to type polymorphisms in genomic DNA samples. These genomic DNA samples used include but are not limited to genomic DNA isolated directly from a plant, cloned genomic DNA, or amplified genomic DNA.

The present invention provides methods for the identification of nucleic acids of interest. In one embodiment, the one or more nucleic acid sequences can be associated with a marker, wherein the marker may be a genetic marker and the genetic marker may comprise some portion of the nucleic acid sequence of interest or be genetically linked to the nucleic acid sequence of interest. Further, an expression profile assay can be used to detect the genetic marker. In one aspect, microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476.

For the purpose of QTL mapping, the markers included should be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes.

As used herein, a “nucleic acid molecule,” be it a naturally occurring molecule or otherwise may be “substantially purified”, if desired, referring to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

The agents of the present invention may also be recombinant. As used herein, the term recombinant means any agent (e.g. DNA, peptide etc.), that is, or results, however indirect, from human manipulation of a nucleic acid molecule.

The agents of the present invention may be labeled with reagents that facilitate detection of the agent (e.g. fluorescent labels (Prober et al. 1987 Science 238:336-340; European Patent 144914), chemical labels (U.S. Pat. No. 4,582,789; U.S. Pat. No. 4,563,417), and modified bases (European Patent 119448).

### 2. Marker-Trait Associations

The present invention provides methods for identification of loci associated with yield using mapping techniques. By establishing performance as a phenotype, genotypes associated with preferred performance are identified. The methods of the present invention are useful for comparing two or more germplasm entries. Exemplary methods for the detection of marker-trait associations are set forth below.

Because of allelic differences in genetic markers, QTL can be identified by statistical evaluation of the genotypes and phenotypes of segregating populations. Processes to map QTL are well-described (WO 90/04651; U.S. Pat. Nos. 5,492,547, 5,981,832, 6,455,758; reviewed in Flint-Garcia et al. 2003 Ann. Rev. Plant Biol. Ann. Rev. Plant Biol. 54:357-374). Methods for determining the statistical significance of a correlation between a phenotype and a genotype, whether a genetic marker or haplotype, may be determined by any statistical test known in the art and with any accepted threshold of statistical significance being required. The application of particular methods and thresholds of significance are well within the skill of the ordinary practitioner of the art. Notably, any type of marker can be correlated with the causative genotype and selection decisions can be made based on a genetic or phenotypic marker.

Using markers to infer a phenotype of interest results in the economization of a breeding program by substituting costly, time-intensive phenotyping with genotyping or a cheaper phenotyping platform, such as an early emerging phenotypic character. Further, breeding programs can be designed to explicitly drive the frequency of specific, favorable phenotypes by targeting particular genotypes (U.S. Pat. No. 6,399,855). Fidelity of these associations may be monitored continuously to ensure maintained predictive ability and, thus, informed breeding decisions (US Patent Application 2005/0015827).

An allele of a QTL can comprise multiple genes or other genetic factors even within a contiguous genomic region or linkage group, such as a haplotype. As used herein, an allele of a QTL or transgene modulating locus can therefore encompass more than one gene or other genetic factor where each individual gene or genetic component is also capable of exhibiting allelic variation and where each gene or genetic factor is also capable of eliciting a phenotypic effect on the quantitative trait in question. In an aspect of the present invention, the allele of a QTL comprises one or more genes or other genetic factors that are also capable of exhibiting allelic variation. The use of the term “an allele of a QTL” is thus not intended to exclude a QTL that comprises more than one gene or other genetic factor. Specifically, an “allele of a QTL” in the present invention can denote a haplotype within a haplotype window wherein a phenotype can be disease resistance. A haplotype window is a contiguous genomic region that can be defined, and tracked, with a set of one or more polymorphic markers wherein the polymorphisms indicate identity by descent. A haplotype within that window can be defined by the unique fingerprint of alleles at each marker. As used herein, an allele is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus. Plants of the present invention may be homozygous or heterozygous at any particular transgene modulating locus or for a particular polymorphic marker.

The identification of marker-trait associations has evolved to the application of genetic markers as a tool for the selection of “new and superior plants” via introgression of preferred genomic regions as determined by statistical analyses (U.S. Pat. No. 6,219,964). Marker-assisted introgression involves the transfer of a chromosomal region, defined by one or more markers, from one germplasm to a second germplasm. The initial step in that process is the localization of the genomic region or transgene by gene mapping, which is the process of determining the position of a gene or genomic region relative to other genes and genetic markers through linkage analysis. The basic principle for linkage mapping is that the closer together two genes are on a chromosome, the more likely they are to be inherited together. Briefly, a cross is generally made between two genetically compatible but divergent parents relative to the traits of interest. Genetic markers can then be used to follow the segregation of these traits in the progeny from the cross, often a backcross (BC1), F₂, or recombinant inbred population.

For the purpose of QTL mapping, the markers included should be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes.

In plant breeding populations, linkage disequilibrium (LD) is the level of departure from random association between two or more loci in a population and LD often persists over large chromosomal segments. Although it is possible for one to be concerned with the individual effect of each gene in the segment, for a practical plant breeding purpose the emphasis is typically on the average impact the region has for the trait(s) of interest when present in a line, hybrid or variety. The amount of pair-wise LD is calculated (using the r² statistic) against the distance in centiMorgan (cM, one hundredth of a Morgan, on average one recombination per meiosis, recombination is the result of the reciprocal exchange of chromatid segments between homologous chromosomes paired at meiosis, and it is usually observed through the association of alleles at linked loci from different grandparents in the progeny) using a set of genetic markers and set of germplasm entries.

The genetic linkage of additional genetic marker molecules can be established by a gene mapping model such as, without limitation, the flanking marker model reported by Lander et al. (Lander et al. 1989 Genetics, 121:185-199), and the interval mapping, based on maximum likelihood methods described therein, and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y.). Use of Qgene software is a particularly preferred approach.

A maximum likelihood estimate (MLE) for the presence of a genetic marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of genetic markers and the length of the genome. Graphs indicating LOD thresholds are set forth in Lander et al. (1989), and further described by Arús and Moreno-González, Plant Breeding, Hayward, Bosemark, Romagosa (eds.) Chapman & Hall, London, pp. 314-331 (1993).

Additional models can be used. Many modifications and alternative approaches to interval mapping have been reported, including the use of non-parametric methods (Kruglyak et al. 1995 Genetics, 139:1421-1428). Multiple regression methods or models can be also be used, in which the trait is regressed on a large number of genetic markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given genetic marker interval, and at the same time onto a number of genetic markers that serve as ‘cofactors,’ have been reported by Jansen et al. (Jansen et al. 1994 Genetics, 136:1447-1455) and Zeng (Zeng 1994 Genetics 136:1457-1468). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng 1994). These models can be extended to multi-environment experiments to analyze genotype-environment interactions (Jansen et al. 1995 Theor. Appl. Genet. 91:33-3). Association study approaches such as transmission disequilibrium tests may be useful for detecting marker-trait associations (Stich et al. 2006 Theor. Appl. Genet. 113:1121-1130).

An alternative to traditional QTL mapping involves achieving higher resolution by mapping haplotypes, versus individual genetic markers (Fan et al. 2006 Genetics 172:663-686). This approach tracks blocks of DNA known as haplotypes, as defined by polymorphic genetic markers, which are assumed to be identical by descent in the mapping population. This assumption results in a larger effective sample size, offering greater resolution of QTL. Methods for determining the statistical significance of a correlation between a phenotype and a genotype, in this case a haplotype, may be determined by any statistical test known in the art and with any accepted threshold of statistical significance being required. The application of particular methods and thresholds of significance are well with in the skill of the ordinary practitioner of the art.

Selection of appropriate mapping populations is important to map construction. The choice of an appropriate mapping population depends on the type of marker systems employed (Tanksley et al., Molecular mapping in plant chromosomes. chromosome structure and function: Impact of new concepts J. P. Gustafson and R. Appels (eds.). Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

An F₂ population is the first generation of selfing after the hybrid seed is produced. Usually a single F₁ plant is selfed to generate a population segregating for all the genes in Mendelian (1:2:1) fashion. Maximum genetic information is obtained from a completely classified F₂ population using a codominant genetic marker system (Mather, Measurement of Linkage in Heredity: Methuen and Co., (1938)). In the case of dominant markers, progeny tests (e.g. F₃, BCF₂) are required to identify the heterozygotes, thus making it equivalent to a completely classified F₂ population. However, this procedure is often prohibitive because of the cost and time involved in progeny testing. Progeny testing of F₂ individuals is often used in map construction where phenotypes do not consistently reflect genotype (e.g. disease resistance) or where trait expression is controlled by a QTL. Segregation data from progeny test populations (e.g. F₃ or BCF₂) can be used in map construction. Marker-assisted selection can then be applied to cross progeny based on marker-trait map associations (F₂, F₃), where linkage groups have not been completely disassociated by recombination events (i.e., maximum disequilibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so. Under conditions of tight linkage (i.e., about <10% recombination), dominant and co-dominant genetic markers evaluated in RIL populations provide more information per individual than either marker type in backcross populations (Reiter et al. 1992 Proc. Natl. Acad. Sci. (USA) 89:1477-1481). However, as the distance between markers becomes larger (i.e., loci become more independent), the information in RIL populations decreases dramatically.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus a population is created consisting of individuals nearly like the recurrent parent but each individual carries varying amounts of genomic regions from the donor parent. Backcross populations can be useful for mapping dominant genetic markers if all loci in the recurrent parent are homozygous and the donor and recurrent parent have contrasting polymorphic marker alleles (Reiter et al. 1992 Proc. Natl. Acad. Sci. (USA) 89:1477-1481). Information obtained from backcross populations using either codominant or dominant markers is less than that obtained from F₂ populations because one, rather than two, recombinant gametes are sampled per plant. Backcross populations, however, are more informative (at low marker saturation) when compared to RILs as the distance between linked loci increases in RIL populations (i.e. about 0.15% recombination). Increased recombination can be beneficial for resolution of tight linkages, but may be undesirable in the construction of maps with low marker saturation.

Near-isogenic lines (NIL) created by many backcrosses to produce an array of individuals that are nearly identical in genetic composition except for the trait or genomic region under interrogation can be used as a mapping population. In mapping with NILs, only a portion of the polymorphic loci are expected to map to a selected region.

Bulk segregant analysis (BSA) is a method developed for the rapid identification of linkage between genetic markers and traits of interest (Michelmore et al. 1991 Proc. Natl. Acad. Sci. (U.S.A.) 88:9828-9832). In BSA, two bulked DNA samples are drawn from a segregating population originating from a single cross. These bulks contain individuals that are identical for a particular trait (resistant or susceptible to particular disease) or genomic region but arbitrary at unlinked regions (i.e. heterozygous). Regions unlinked to the target region will not differ between the bulked samples of many individuals in BSA.

In another embodiment, plants can be screened for one or more markers associated using high throughput, non-destructive seed sampling, wherein one or more of the markers is associated with at least one transgene modulating locus. In a preferred aspect, seed is sampled in this manner and only seed with at least one genotype of interest is advanced. Apparatus and methods for the high-throughput, non-destructive sampling of seeds have been described which would overcome the obstacles of statistical samples by allowing for individual seed analysis. For example, U.S. patent application Ser. No. 11/213,430 (filed Aug. 26, 2005); U.S. patent application Ser. No. 11/213,431 (filed Aug. 26, 2005); U.S. patent application Ser. No. 11/213,432 (filed Aug. 26, 2005); U.S. patent application Ser. No. 11/213,434 (filed Aug. 26, 2005); and U.S. patent application Ser. No. 11/213,435 (filed Aug. 26, 2005), U.S. patent application Ser. No. 11/680,611 (filed Mar. 2, 2007), which are incorporated herein by reference in their entirety, disclose apparatus and systems for the automated sampling of seeds as well as methods of sampling, testing and bulking seeds.

### 3. Plant Breeding

Plants of the present invention can be part of or generated from a breeding program. The choice of breeding method depends on the mode of plant reproduction, the heritability of the trait(s) being improved, and the type of cultivar used commercially (e.g., F₁ hybrid cultivar, pureline cultivar, etc). A cultivar is a race or variety of a plant species that has been created or selected intentionally and maintained through cultivation.

The present invention provides for parts of the plants of the present invention.

Selected, non-limiting approaches for breeding the plants of the present invention are set forth below. A breeding program can be enhanced using marker assisted selection (MAS) on the progeny of any cross. It is understood that nucleic acid markers of the present invention can be used in a MAS (breeding) program. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

In one aspect, MAB programs use a plurality of markers to identify higher performing selections that have, on average, a higher frequency of favorable alleles at one or more loci. Fingerprinting was developed to determine the genome-wide marker distribution. Using the resulting marker distance and/or marker similarities indices between two or more lines, it is possible to build pedigrees and to calculate the breeding value across all assessed loci. Herein, breeding values are calculated based on expression profile effect estimates and expression profile (i.e., allele) frequency, wherein the expression profile breeding value represents the effect of fixing a particular nucleic acid sequence (i.e., allele) underlying the expression profile in a population, thus providing the basis for ranking nucleic acid sequences, based on corresponding expression profiles.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred aspect, a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes.

Breeding lines can be tested and compared to appropriate standards in environments representative of the commercial target area(s) for two or more generations. The best lines are candidates for new commercial cultivars; those still deficient in traits may be used as parents to produce new populations for further selection.

For hybrid crops, the development of new elite hybrids requires the development and selection of elite inbred lines, the crossing of these lines and selection of superior hybrid crosses. The hybrid seed can be produced by manual crosses between selected male-fertile parents or by using male sterility systems. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Backcross breeding has been used to transfer genes for a simply inherited, highly heritable trait into a desirable homozygous cultivar or inbred line, which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have most attributes of the recurrent parent (e.g., cultivar) and, in addition, the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

The doubled haploid (DH) approach achieves isogenic plants in a shorter time frame. DH plants provide an invaluable tool to plant breeders, particularly for generating inbred lines and quantitative genetics studies. For breeders, DH populations have been particularly useful in QTL mapping, cytoplasmic conversions, and trait introgression. Moreover, there is value in testing and evaluating homozygous lines for plant breeding programs. All of the genetic variance is among progeny in a breeding cross, which improves selection gain.

Most research and breeding applications rely on artificial methods of DH production. The initial step involves the haploidization of the plant which results in the production of a population comprising haploid seed. Non-homozygous lines are crossed with an inducer parent, resulting in the production of haploid seed. Seed that has a haploid embryo, but normal triploid endosperm, advances to the second stage. That is, haploid seed and plants are any plant with a haploid embryo, independent of the ploidy level of the endosperm.

After selecting haploid seeds from the population, the selected seeds undergo chromosome doubling to produce doubled haploid seeds. A spontaneous chromosome doubling in a cell lineage will lead to normal gamete production or the production of unreduced gametes from haploid cell lineages. Application of a chemical compound, such as colchicine, can be used to increase the rate of diploidization. Colchicine binds to tubulin and prevents its polymerization into microtubules, thus arresting mitosis at metaphase, can be used to increase the rate of diploidization, i.e. doubling of the chromosome number These chimeric plants are self-pollinated to produce diploid (doubled haploid) seed. This DH seed is cultivated and subsequently evaluated and used in hybrid testcross production.

Descriptions of other breeding methods that are commonly used for different traits and crops can be found in one of several reference books (Allard, “Principles of Plant Breeding,” John Wiley & Sons, NY, U. of CA, Davis, Calif., 50-98, 1960; Simmonds, “Principles of crop improvement,” Longman, Inc., NY, 369-399, 1979; Sneep and Hendriksen, “Plant breeding perspectives,” Wageningen (ed), Center for Agricultural Publishing and Documentation, 1979; Fehr, In: Soybeans: Improvement, Production and Uses, 2nd Edition, Monograph., 16:249, 1987; Fehr, “Principles of variety development,” Theory and Technique, (Vol. 1) and Crop Species Soybean (Vol. 2), Iowa State Univ., Macmillan Pub. Co., NY, 360-376, 1987).

In a preferred aspect of the present invention, an expression profile is determined for at least one locus in at least one plant genome and the expression profile is associated with at least one phenotypic trait. The material evaluated can include at least one inbred plant, at least one hybrid plant, and at least one tester. In certain aspects, at least two alleles for at least one locus are evaluated. In other aspects, at least two expression variants for at least one allele are evaluates. Methods for calculating associations between two or more data sets are known in the art. For example, in multiple regression analysis, a linear equation is fit so as to describe the response of one or more dependent variables to variation in a set of explanatory variables. In another aspect, ordinary least-squares regression can be used, wherein the objective is to minimize the prediction error of response in the sample of response and explanatory variables under immediate consideration. Another method, partial least-squares (PLS) regression, has the objective of minimizing prediction error of response when the linear prediction equation is applied to a new sample drawn from the same population from which the original sample was obtained.

When the explanatory variables are highly correlated, least-squares regression performs very poorly in prediction of dependent variable response in future samples. Partial least-squares multiple regression overcomes this deficiency by focusing on directions in the predictor space of response and explanatory variables that are well sampled. The well-sampled directions are determined by factorization of the covariance matrix of the response and explanatory variables. Cross validation is then employed to determine which factors are best sampled and, therefore, are the best predictors of response.

In another aspect, the partial least-squares technique can be used. The first applications of PLS regression were to problems in fitting calibrated response curves of chemical elements in a sample to highly collinear spectral emission data. A similar problem arises when one attempts to fit observed phenotypic variation in yield to variation in gene expression or variation in marker genotype. Gene expression or marker variables can, by their very nature, be highly collinear due to linkage disequilibrium between loci physically linked along the chromosomes. In addition, many more gene expression and marker variables are usually available than can be accommodated by any reasonable sample of observations. For ordinary least-squares, this means that the normal equations will be comprised of a set of consistent equations that have an infinite number of solutions, any one of which is perfect for the sample at hand, but of absolutely no use in predicting response in future samples. PLS regression circumvents this problem by identifying a very few highly predictive factors, many fewer than the number of observations in the sample.PLS regression has been shown to perform very well on small samples with very large numbers of explanatory variables (Boulesteix and Strimmer 2006 Briefings in Bioinformatics 8: 32-4). Consequently, it was a natural choice for exploring the relationships between phenotypic variation in yield with variation in gene expression and marker genotype.

Once the predictive factors are identified, the regression coefficients on these factors are transformed back into the space of the original explanatory variables. Then, selection among the original variables to attain an even more predictive model can be accomplished by calculation of the VIP (Variable Importance for Prediction) statistic. It can be shown, through calculation of the empirical distribution of the statistic, that a cutoff value of VIP=0.8 is generally appropriate for explanatory variable selection. That is, when the cutoff is imposed, only explanatory variables with VIP values equal to or greater than 0.8 are retained in the regression model.

A number of versions of PLS regression are known. One of the most useful is the SIMPLS routine (dejong 1993 Chemometrics and Intelligent Laboratory Systems 18:251-263). The utility of this version is due to the fact that different models constituted with different explanatory variables are directly comparable. In accordance with this benefit, the SIMPLS version of SAS Proc PLS was employed in all PLS regression applications described below.

Gain from selection on a trait is dependent on selection intensity, the amount of genetic variability for the trait in the population under selection, and the repeatability of the trait over diverse environmental conditions. Repeatability of the trait is measured as the ratio of genetic variation to phenotypic variation, where phenotypic variation is the sum total of genetic and environmental variation.

Selection can be applied simultaneously to multiple traits. For multiple traits selection, the equation for expected genetic advance, or gain, is, in matrix notation

ΔH=k(B´Gw)^{1/2}

where

B=P⁻¹Gw

and

k=selection intensity
G=the genotypic covariance matrix among traits
P=the phenotypic covariance matrix among traits
w=weight vector with elements assigned according to the importance of the traits.

Multiple trait selection as described by the equations above is called index selection (Falconer 1960 Introduction to quantitative genetics. Ronald Press, NY), with the index determined by the elements of the w vector assigned in accordance with the breeder's estimation of the importance of the traits.

Genetic and phenotypic correlations are derived from the G and P matrices, respectively, while the repeatabilities (also called heritabilities) are obtained from the ratios of the G and P matrix diagonal elements.

For selection on a single trait, the G and P matrices and the B vector devolve into scalar quantities, while the w vector becomes just a scalar equal to 1.

For correlated response in one trait to selection on the others, the G matrix becomes a row vector corresponding to the response trait, the element of the w vector corresponding to the response trait is set equal to zero while the elements corresponding to the traits under selection are set equal to one, and the P matrix contains only rows and columns corresponding to the traits under selection.

To examine the efficacy of including probeset and marker models of predicted yield in selection for yield, three scenarios may be envisioned: (1) selection on observed yield only; (2) correlated response in yield to selection on probeset or marker model predicted yield; (3) index selection in which the element of the weight matrix corresponding to observed yield is set equal to 1 and the element corresponding to model predicted yield is set equal to zero. Scenario (2) is often referred to as indirect selection, and scenario (3) is known as index selection with a secondary trait. The secondary trait is given a weight of zero in the index since it of itself has no value, but if its repeatability is high and its genetic correlation with the primary trait is substantial, it can be an effective aid in selection for the primary trait.

Given finished sequence of the maize genome, one could first identify markers proximal to eQTL but in trans with respect to a probeset significantly associated with a phenotypic trait, locate the marker on a BAC or contig, scan the region for open reading frames (ORF), blast sequence from the ORFs against gene data bases for possible function, and then lay down candidate oligos on a new chip for a second round of discovery. Obviously, the cycle could be extended to three or more rounds.

The methods of the present invention take advantage of recent technology developments in the field of expression profiling assays, e.g., U.S. Pat. No. 7,163,792, U.S. Pat. No. 7,081,339, U.S. Pat. No. 6,996,476, U.S. Pat. No. 6,548,257, US Patent Application 20070172854, US Patent Application 20070172831, US Patent Application 20070148690, US Patent Application 20070037189, and US Patent Application 20070003953.

As provided by the present invention, the knowledge of nucleic acid sequences as inferred by expression profiles targeting the nucleic acid sequences can be applied to make decisions at multiple stages of the breeding program:

a) Among segregating progeny, as a pre-selection method, to increase the selection index and drive the frequency of favorable nucleic acid sequences among breeding populations, wherein pre-selection is defined as selection among offspring of a breeding cross based on the genotype of these progenies at a selected set of two or more nucleic acid sequences, and leveraging of expression profile-trait associations identified in previous breeding crosses.
b) Among segregating progeny from a breeding population, to increase the frequency of the favorable nucleic acid sequences for the purpose of line or variety development.
c) Among segregating progeny from a breeding population, to increase the frequency of the favorable nucleic acid sequences prior to QTL mapping within this breeding population.
d) For hybrid crops, among parental lines from different heterotic groups to predict the performance potential of different hybrids.

In another embodiment, the present invention provides a method for improving plant germplasm by accumulation of nucleic acid sequences of interest in a germplasm comprising determining expression profiles for at least two loci in the genome of a species of plant, and associating the expression profiles with at least one trait, and using this expression profile effect estimates to direct breeding decisions. These expression profile effect estimates can be derived using historical expression profile-trait associations or de novo from mapping populations. The expression profile effect estimates for one or more traits provide the basis for making decisions in a breeding program. This invention also provides an alternative basis for decision-making using breeding value calculations based on the estimated effect and frequency of the underlying nucleic acid sequences in the germplasm. These breeding values can be used to rank a specified set of nucleic acid sequences. In the context of the specified set of nucleic acid sequences, these breeding values form the basis for calculating an index to rank the alleles both within and between loci.

For example, any given chromosome segment can be represented in a given population by a number of nucleic acid sequences that can vary from 1 (region is fixed), to the size of the population times the ploidy level of that species (2 in a diploid species), in a population in which every chromosome has a different nucleic acid sequence. Identity-by-descent among nucleic acid sequences carried by multiple individuals in a non-fixed population will result in an intermediate number of different nucleic acid sequences and possibly a differing frequency among the different nucleic acid sequences. New nucleic acid sequences may arise, through recombination at meiosis between existing nucleic acid sequences in heterozygous progenitors. The frequency of each nucleic acid sequence may be estimated by several means known to one versed in the art (e.g. by direct counting, or by using an EM algorithm). Let us assume that “k” different nucleic acid sequences, wherein a nucleic acid sequence represents at least one nucleotide and may constitute an allele or haplotype, identified as “nᵢ” (i=1, . . . , k), are known, that their frequency in the population is “fᵢ” (i=1, . . . , k), and for each of these nucleic acid sequences we have an effect estimate “Estᵢ” (i=1, . . . , k). If we call the “breeding value” (BVᵢ) the effect on that population of fixing that nucleic acid sequence, then this breeding value corresponds to the change in mean for the trait(s) of interest of that population between its original state of haplotypic distribution at the window and a final state at which nucleic acid sequence “nᵢ” encounters itself at a frequency of 100%. The breeding value estimate of nᵢ in this population can be calculated as:

$${BV}_{i} = {Est}_{i} - {\sum }_{i = 1}^{k} {Est}_{i} {f}_{i} .$$

One skilled in the art will recognize that nucleic acid sequences that are rare in the population in which effects are estimated tend to be less precisely estimated, this difference of confidence may lead to adjustment in the calculation. For example one can ignore the effects of rare nucleic acid sequences, by calculating breeding value of better known nucleic acid sequence after adjusting the frequency of these (by dividing it by the sum of frequency of the better known nucleic acid sequences). One could also provide confidence intervals for the breeding value of each nucleic acid sequences.

This breeding value will change according to the population for which it is calculated, as a function of difference of nucleic acid sequence frequencies. The term population can then assume different meanings, below are two examples of special cases. First, it can be a single inbred line in which one intend to replace its current nucleic acid sequence nⱼ by a new nucleic acid sequence nᵢ, in this case BVᵢ=Estᵢ-Estⱼ. Second, it can be a F2 population in which the two parental nucleic acid sequence nᵢ and nⱼ are originally present in equal frequency (50%), in which case BVᵢ=½ (Estᵢ-Estⱼ).

These statistical approaches enable expression profile effect estimates to inform breeding decisions in multiple contexts. Other statistical approaches to calculate breeding values are known to those skilled in the art and can be used in substitution without departing from the spirit and scope of this invention.

Further, methods for determining the statistical significance of a correlation between a phenotype and a genotype, in this case an expression profile, may be determined by any statistical test known in the art and with any accepted threshold of statistical significance being required. The application of particular methods and thresholds of significance are well with in the skill of the ordinary practitioner of the art.

Expression profile effect estimates and/or breeding values for one or more traits of interest provide the basis for determining one or more nucleic acid sequences of interest in comparisons of two or more expression profiles. With this a priori information, breeding selections are conducted on a nucleic acid sequence basis, wherein a first plant is crossed with a second plant that contains at least one nucleic acid sequence that is different from the first plant nucleic acid sequence or nucleic acid sequences; and at least one progeny plant is selected by detecting the nucleic acid sequence or set of nucleic acid sequences of the first plant, wherein the progeny plant comprises in its genome one or more nucleic acid sequences of interest of the first plant and at least one nucleic acid sequence of interest of the second plant; and the progeny plant is used in activities related to germplasm improvement, herein defined as including using the plant for line and variety development, hybrid development, transgenic event selection, making breeding crosses, testing and advancing a plant through self fertilization, using plant or parts thereof for transformation, using plants or parts thereof for candidates for expression constructs, and using plant or parts thereof for mutagenesis.

In one aspect, this invention uses expression profiles to identify nucleic acids, in one or more regions of a plant genome, that provide a basis to compare two or more germplasm entries. Regions of contiguous nucleic acid sequence are indicative of the conservation of genetic identity of all intervening genes from a common progenitor. In cases where conserved sequence segments are coincident with segments in which QTL have been identified it is possible to deduce with high probability that QTL inferences can be extrapolated to other germplasm having an identical sequence in that locus. This a priori information provides the basis to select for favorable QTLs prior to QTL mapping within a given population.

For example, plant breeding decisions could comprise:

a) Selection among new breeding populations to determine which populations have the highest frequency of favorable nucleic acid sequences, wherein sequences are designated as favorable based on coincidence with previous QTL mapping; or
b) Selection of progeny containing said favorable nucleic acid sequences in breeding populations prior to, or in substitution for, QTL mapping within that population, wherein selection could be done at any stage of breeding and could also be used to drive multiple generations of recurrent selection; or
c) Prediction of progeny performance for specific breeding crosses; or
d) Selection of lines for germplasm improvement activities based on said favorable nucleic acid sequences, including line development, hybrid development, selection among transgenic events based on the breeding value of the haplotype that the transgene was inserted into, making breeding crosses, testing and advancing a plant through self fertilization, using plant or parts thereof for transformation, using plants or parts thereof for candidates for expression constructs, and using plant or parts thereof for mutagenesis.

An additional unique aspect of this invention is the ability to select for specific genes or gene alleles, as they are targeted by expression profile assays. For example, in cases where the target nucleic acid sequence is coincident with segments in which genes have been identified it is possible to deduce with high probability that gene inferences can be extrapolated to other germplasm having an identical genotype in that locus. This a priori information provides the basis to select for favorable genes or gene alleles on the basis of an expression profile assay comprising the target nucleic acid within a given population.

For example, plant breeding decisions could comprise:

a) Selection among new breeding populations to determine which populations have the highest frequency of favorable nucleic acid sequences, wherein sequences are designated as favorable based on coincidence with previous gene mapping; or
b) Selection of progeny containing said favorable nucleic acid sequences in breeding populations, wherein selection is effectively enabled at the gene level, wherein selection could be done at any stage of inbreeding and could also be used to drive multiple generations of recurrent selection; or
c) Prediction of progeny performance for specific breeding crosses; or
d) Selection of lines for germplasm improvement activities based on said favorable nucleic acid sequences, including line development, hybrid development, selection among transgenic events based on the breeding value of the haplotype that the transgene was inserted into, making breeding crosses, testing and advancing a plant through self fertilization, using plant or parts thereof for transformation, using plants or parts thereof for candidates for expression constructs, and using plant or parts thereof for mutagenesis.

Further, in another preferred embodiment of this invention, the a priori information on the frequency of favorable nucleic acid sequences, as identified by an expression profile assay, in breeding populations enables pre-selection. That is, parental lines are selected based on the historical genotype-phenotype association information, wherein the genotype comprises an expression profile, for the purpose of driving favorable nucleic acid frequency for multiple traits simultaneously. In pre-selection, breeders can predict the phenotypic contribution for multiple traits of any line based on that line's fingerprint information, which corresponds to a composition of pre-defined expression profiles and the corresponding nucleic acid sequences. This multi-trait sequence selection approach economizes a breeding program by initiating selection at the initial stage of choosing parental crosses and it also reduces the need for costly, time-consuming phenotyping of progeny.

A preferred sequence provides a preferred property to a parent plant and to the progeny of the parent when selected by a marker means or phenotypic means. The method of the present invention provides for selection of preferred sequences, or sequences of interest, and the accumulation of these sequences in a breeding population.

In another embodiment, this invention enables indirect selection through selection decisions for at least one nucleic acid sequence based on at least one expression profile effect estimate such that additional phenotypes are indirectly selected upon due to the additional expression profile effect estimates for other phenotypic traits.

Another preferred embodiment of the present invention is to build additional value by selecting a composition of nucleic acid sequences wherein each corresponding expression profile has an estimated associated phenotype that is not negative with respect to yield, or is not positive with respect to maturity, or is null with respect to maturity, or amongst the best 50 percent with respect an agronomic trait, transgene, and/or a multiple trait index when compared to any other nucleic acid sequence at the same locus in a set of germplasm, or amongst the best 50 percent with respect to an agronomic trait, transgene, and/or a multiple trait index when compared to any other loci across the entire genome in a set of germplasm, or the nucleic acid sequence being present with a frequency of 75 percent or more in a breeding population or a set of germplasm can be taken as evidence of its high value, or any combination of these.

This invention anticipates a stacking of nucleic acid sequences from at least two loci into plants or lines by crossing parent plants or lines containing different nucleic acid sequences with different corresponding expression profiles, that is, different genotypes. The value of the plant or line comprising in its genome stacked nucleic acid sequences from two or more loci can be estimated by a composite breeding value, which depends on a combination of the value of the traits and the value of the nucleic acid sequence(s) to which the traits are linked. The present invention further anticipates that the composite breeding value of a plant or line can be improved by modifying the components of one or each of the nucleic acid sequences. Additionally, the present invention anticipates that additional value can be built into the composite breeding value of a plant or line by selection of at least one recipient nucleic acid sequence with a preferred expression profile effect estimate or, in conjunction with the frequency of said nucleic acid sequence in the germplasm pool, breeding value to which one or any of the other nucleic acid sequences are linked, or by selection of plants or lines for stacking two or more nucleic acid sequences from two or more loci by breeding.

Another embodiment of this invention is a method for enhancing breeding populations by accumulation of one or more nucleic acid sequences in one or more loci, in a germplasm. Loci include genetic information and provide phenotypic traits to the plant. Variations in the genetic information can result in variation of the phenotypic trait and the value of the phenotype can be measured. The genetic mapping of the nucleic acid sequences allows for a determination of linkage across sequences. The nucleic acid sequence of interest is novel in the genome of the progeny plant and can in itself serve as a genetic marker of a locus of interest. Notably, this nucleic acid sequence can also be used as an identifier for a gene or QTL. For example, in the event of multiple traits or trait effects associated with the nucleic acid sequence, only one marker would be necessary for selection purposes. Additionally, the locus of interest may provide a means to select for plants that have the linked locus.

In another embodiment, the present invention provides methods and compositions for biomarkers. Those markers might be useful for optimizing screening conditions. A biomarker is a characteristic that can be used as an indicator of biologic processes or responses to internal and external interventions. Herein, characteristic gene expression profiles can be related to different biological processes and stress responses, such as drought and nitrogen deficiency. The information is not only useful for understanding the underlying molecular mechanisms and identifying lead genes, but also can be used to develop biomarkers as indicators for various purposes. In this context, the TxP biomarkers under investigation are the genes with indicative expression profiles for the following aspects:

a. Biological pathways (e.g. ABA biosynthesis): these include key pathway genes that indicate a particular mode of action that a transgene may have, or a given stress type may affect.
b. Stress responses (e.g. nitrogen deficiency): These biomarkers may monitor the types and severity of stresses imposed on plants. They can be used for evaluation and optimization of screening conditions.
c. Transgene activity: They are indicators for cellular effect of transgenes and could be used in event evaluation and selection. They can also provide the possible MOA for the transgenes.
d. Physiology efficacy: They are indicators for physiology aspects of plants, which could replace for certain types of greenhouse or field assays.
e. Yield efficacy: They are predicators of yield efficacy with strong association with field test result for individual genes or gene groups under specific or general conditions. They could be used to aid event selection to increase the hit rate and save field testing cost and time.
f. Trait (e.g. oil content): These are the biomarkers associated with specific traits not necessarily in transgenic plants. They could be used as complement to genetic markers for breeding selection in given germplasm populations.
g. Consistency of Treatment: These are the biomarkers which evaluate stress and drought tolerance, monitoring of environmental microheterogeneity, defining and classification of microenvironments, providing increased precision in data analyses and to aid decisions for plant advancement.

The present invention also provides for the screening of progeny plants' loci of interest and using the expression profile effect estimate as the basis for selection for use in a breeding program to enhance the accumulation of preferred nucleic acid sequences.

Using this method, the present invention contemplates that nucleic acid sequences of interest are selected from a large population of plants. Additionally, these nucleic acid sequences can be used in the described breeding methods to accumulate other beneficial and preferred loci and maintain these in a breeding population to enhance the overall germplasm of the crop plant. Crop plants considered for use in the method include but are not limited to, corn, soybean, cotton, wheat, rice, canola, oilseed rape, sugar beet, sorghum, millet, alfalfa, forage crops, oilseed crops, grain crops, fruit crops, ornamental plants, vegetable crops, fiber crops, spice crops, nut crops, turf crops, sugar crops, beverage crops, tuber crops, root crops, and forest crops.

The present invention provides methods of use for a portable, isothermal nucleic acid detection technology. Applications include methods for nucleic acid screening during breeding. This genotyping capability can be used for pre-selection, progeny selection, and stacking of genomic traits. In particular, this is especially useful for genotyping “on the fly” wherein potential resource efficiency is possible by only growing to maturity plants comprising one or more preferred genotypes. In one aspect, seedlings are raised in a greenhouse, leaf tissue is sampled, and only preferred plants are further propagated, either in the greenhouse or transplanted to the field. In particular, this tool is used in a multi-season program (i.e., winter nursery) where facilities and resources are not conducive to a typical lab-based genotyping facility but this tool allows for genotypic data to be collected and subsequently populated in a database, allowing decision-making by the breeder regardless of his/her geographic location.

### B. Transgenic Breeding

In still another embodiment, the present invention acknowledges that preferred nucleic acids identified by the methods presented herein may be advanced as candidate genes for inclusion in expression constructs, i.e., transgenes.

Nucleic acids underlying expression profiles of interest may be expressed in plant cells by operably linking them to a promoter functional in plants. Nucleic acids for proteins disclosed in the present invention can be expressed in plant cells by operably linked them to a promoter functional in plants Tissue specific and/or inducible promoters may be utilized for appropriate expression of a nucleic acid for a particular trait. The 3′ un-translated sequence, 3′ transcription termination region, or poly adenylation region means a DNA molecule linked to and located downstream of a structural polynucleotide molecule responsible for a trait and includes polynucleotides that provide polyadenylation signal and other regulatory signals capable of affecting transcription, mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA genes. A 5′ UTR that functions as a translation leader sequence is a DNA genetic element located between the promoter sequence and the coding sequence. The translation leader sequence is present in the fully processed mRNA upstream of the translation start sequence. The translation leader sequence may affect processing of the primary transcript to mRNA, mRNA stability or translation efficiency.

In another aspect, nucleic acids underlying expression profiles of interest may have their expression modified by double-stranded RNA-mediated gene suppression, also known as RNA interference (“RNAi”), which includes suppression mediated by small interfering RNAs (“siRNA”), trans-acting small interfering RNAs (“ta-siRNA”), or microRNAs (“miRNA”). Examples of RNAi methodology suitable for use in plants are described in detail in U.S. patent application publications 2006/0200878 and 2007/0011775.

Methods are known in the art for assembling and introducing constructs into a cell in such a manner that the nucleic acid molecule for a trait is transcribed into a functional mRNA molecule that is translated and expressed as a protein product. For the practice of the present invention, conventional compositions and methods for preparing and using constructs and host cells are well known to one skilled in the art, see for example, Molecular Cloning: A Laboratory Manual, 3rd edition Volumes 1, 2, and 3 (2000) J. F. Sambrook, D. W. Russell, and N. Irwin, Cold Spring Harbor Laboratory Press. Methods for making transformation constructs particularly suited to plant transformation include, without limitation, those described in U.S. Pat. Nos. 4,971,908, 4,940,835, 4,769,061 and 4,757,011, all of which are herein incorporated by reference in their entirety. Transformation methods for the introduction of expression units into plants are known in the art and include electroporation as illustrated in U.S. Pat. No. 5,384,253; microprojectile bombardment as illustrated in U.S. Pat. Nos. 5,015,580; 5,550,318; 5,538,880; 6,160,208; 6,399,861; and 6,403,865; protoplast transformation as illustrated in U.S. Pat. No. 5,508,184; and Agrobacterium-mediated transformation as illustrated in U.S. Pat. Nos. 5,635,055; 5,824,877; 5,591,616; 5,981,840; and 6,384,301.

In summary, this invention describes the novel combination of expression profile analysis and molecular breeding methodologies to enable the use of expression profile information to carry out molecular plant breeding. Taken together, this invention enables the plant breeder to use expression profile information in parent selection, progeny selection, choosing tester combinations, developing pedigrees, fingerprinting samples, screening for haplotype diversity, and for building databases of sequence associations to trait and performance data.

### EXAMPLE 1

### Association of Genetic Variation in Yield with Transcript Expression and Expression QTL in Testcrosses of Homozygous Inbred Lines of Maize

The present example provides the results and analyses used to identify transcript profiles and SNP markers associated with yield in maize. SEQ IDs are provided for the ORF corresponding to probesets associated with yield, wherein these nucleic acids are useful both as molecular markers for either SNP development or sequence-assisted breeding (U.S. Patent Application Ser. No. 60/942,707) and as nucleic acid expression constructs for use in transgenic breeding. Further, data are providing that support the value of these nucleic acid sequences for the prediction of yield, via regression analyses for probesets, expression QTL (eQTL) and combinations thereof.

The plant materials used were testcrosses of a set of maize inbred lines crossed to two testers. The inbred line set was comprised of 82 dihaploid progeny emanating from random crosses among a large number of parental inbreds belonging to the female heterotic group. The dihaploid progeny were comprised of sib-pair or single-individuals families, each emanating from a different cross. The testers were comprised of two inbred lines. The tester lines are unrelated to each other and to all of the lines in the dihaploid progeny set. Testcrosses of 80 dihaploid lines by inbred A, and two dihaploid lines by inbred B, were evaluated at three locations in central Iowa over two years. Ear leaf tissue was sampled from each hybrid at each location at anthesis, flash frozen in liquid nitrogen and stored in frozen condition. The leaf samples were then fine-ground and m-RNA extracted. The extracted m-RNA from each sample was labeled with a fluorescent dye and hybridized to an Affymetrix GeneChip containing 2204 probesets. Each probeset was comprised of 16 25-mer oligonucleotide probes, each probeset representing an expressed sequence tag (EST) from a unigene library. The 2204 probesets were, on the basis of earlier studies, or probeset annotation, thought to be representative of genes associated with yield variation.

To measure gene expression associated with a particular probeset in a sample in a TxP experiment, first mRNA is extracted from the sample, cRNA is transcribed, then sheared to an average size of approximately 100 bases, and then labeled with a fluor. The labeled sheared cRNA prep is then hybridized to the chip, and the level of hybridization as measured by fluorescence is recorded for each of the 1.3 million probes on the chip. Fluorescence intensity from each probe of the probeset was recorded, and the composite fluorescence intensity, estimated as the Tukey mean of all probes in the probeset, was calculated. Fluorescence intensity is assumed to be proportional to mRNA concentration in the sample and, hence, is an indicator of transcriptional activity of the gene represented by the probeset. Transcript activity is commonly referred to as gene transcript expression (TxP).

Testcrosses, to inbred B and in which ear leaf samples had been taken at anthesis, of sixty-five members of the dihaploid line set had been evaluated in year 1. These testcrosses also had been evaluated at three central Iowa locations. Leaf samples were collected and processed in year 1 in a manner identical to that in year 2.

Each of the dihaploid lines had been genotyped for a large number of SNP markers. Markers from 653 SNP loci were select for analysis in the study; SNP markers of the present invention are provided in US Patent Application 20060141495 and WO 2008/021225, both incorporated herein by reference in their entirety. The criteria for selection comprised: (1) minimal missing data and (2) informativeness.

All plots in both years were machine-harvested, and grain yield in bushels per acre adjusted to %15.5 grain moisture was recorded.

### Least-Squares Means of Yield and Gene Expression

In the data structure of the study, the experimental design was unbalanced to some extent. Consequently, for further analysis, a line least-squares mean for yield, and for gene expression of each probeset, was calculated using SAS Proc GLM. Values of least-squares means are those which would be obtained if the experimental design was balanced. The model used for the calculation of the least-squares means was

y=location+line+residual

where y stands either for yield or for gene expression of a particular probeset.

Gene expression for each probeset can be regarded as a phenotypically observed trait, no differently than for yield. Hence, hereinafter, ‘probeset’ will variously be understood to mean the name of the probeset or the line least-squares mean for gene expression of the probeset. Which meaning is appropriate will be understood from the context. Also, hereinafter, ‘yield’ will be understood to mean the line least-squares mean of yield.

### Single-Factor Regression Analysis

Simple single-factor regressions of yield on each probeset and yield on each SNP marker locus were calculated. In addition, Single-factor regressions of each probeset on each SNP marker locus were calculated.

The model for each regression was of the form

y=a+xb+residual

where y stands either for the yield or probeset expression, a is the intercept, x is either probeset expression or SNP genotype (coded as 1 for allele 1, −1 for allele 2, and 0 for missing), b is the regression coefficient, and residual is deviation from regression. An F test for significance of the regression coefficient was calculated for each regression analysis. Because, many regression analyses were calculated for yield and each probeset, false positives due to multiple testing needed to be taken into account. To guard against false positives, the false discovery rate (FDR) was calculated (Benjamini and Hochberg 1995) in addition to the F test for significance in each single-factor analysis. Because the results from the single-factor analyses are quite voluminous, they are not presented here but can be accessed from the SAS data sets Single_Factor_M and Single_Factor_P data sets.

A set of 68 probesets that, on the basis of single factor analysis of probeset expression on marker genotype, mapped very closely to one or more marker loci.

### Regression of Yield on Probesets

A summary of the results of PLS regression of yield on the 68 probesets appears in Table 1. The rsquare value for the model was 47%, meaning that variation in the 68 probesets explained 47% of the variation in yield. The inference, under the assumptions relevant to PLS regression, is that the model comprised of the 68 probesets is the most predictive model for prediction of yield in future samples which are drawn from the same population of testcrosses, and which are evaluated across a sample of environments drawn from the same population of environments typical of those in the present sample. Also reported is the variable importance predictor (VIP) as measured in SAS. This value indicated the value of the probeset as a predictor of yield. VIP values greater than 0.8 are considered indicative of strong predictive power.

TABLE 1
Significant probesets in partial least squares regression of yield on probeset
expression. SEQ IDs include the nucleic acid sequence for the corresponding ORF
sequence for the probeset. Rsquare for the regression model = 47.47.

| | | | Probeset | |
|---|---|---|---|---|
| | SEQ | Probeset | Regression | |
| | ID | Regression | Coefficient | Putative Probeset |
| Probeset | NO | Coefficient | VIP | Annotation |
| A1ZM005469_AT | 367 | 0.01944 | 0.95 | |
| A1ZM047966_X_AT | 368 | −0.01817 | 0.89 | putative 60S ribosomal |
| | | | | protein L37 |
| A1ZM055950_S_AT | 369 | 0.01973 | 0.97 | |
| A1ZM049194_AT | 370 | 0.02061 | 1.01 | sugar transporter |
| | | | | family protein |
| A1ZM002154_S_AT | 371 | 0.01976 | 0.97 | unknown |
| A1ZM024567_AT | 372 | 0.01811 | 0.89 | DJ-1 family protein |
| A1ZM069545_AT | 373 | 0.01914 | 0.94 | |
| A1ZM001187_A_AT | 374 | 0.02095 | 1.03 | RING zinc finger |
| | | | | protein-like |
| A1ZM003299_S_AT | 375 | 0.02117 | 1.04 | ESTs |
| | | | | C98382(C2985), D22444 |
| | | | | (C11129) correspond |
| | | | | to a region of the |
| | | | | predicted gene.~Similar |
| | | | | to Arabidopsis thaliana |
| | | | | APK1 gene for protein |
| | | | | tyrosine-serine- |
| | | | | threonine |
| | | | | kinase.(D12522) |
| A1ZM061606_X_AT | 376 | −0.01940 | 0.95 | OSJNBa0079A21.14 |
| A1ZM067500_AT | 377 | −0.01933 | 0.95 | NADH-ubiquinone |
| | | | | oxidoreductase 24 kDa |
| | | | | subunit, putative |
| A1ZM079744_AT | 378 | −0.02280 | 1.12 | |
| A1ZM025769_AT | 379 | 0.02453 | 1.20 | putative chloroplast |
| | | | | membrane-associated |
| | | | | 30 kD protein |
| A1ZM015563_AT | 380 | −0.01863 | 0.92 | OSJNBa0084K11.5 |
| A1ZM033956_AT | 381 | −0.01912 | 0.94 | |
| A1ZM044683_X_AT | 382 | 0.02033 | 1.00 | |
| A1ZM039148_AT | 383 | −0.02143 | 1.05 | |
| A1ZM045717_AT | 384 | −0.01890 | 0.93 | EBNA-3B |
| A1ZM081073_AT | 385 | −0.01906 | 0.94 | |
| A1ZM025661_AT | 386 | −0.02022 | 0.99 | 12-oxophytodienoic |
| | | | | acid reductase |
| A1ZM030937_AT | 387 | −0.02380 | 1.17 | contains ESTs |
| | | | | AU093915(E1276), AU162319 |
| | | | | (E60301)~similar |
| | | | | to cytosolic |
| | | | | factor~unknown |
| | | | | protein |
| A1ZM058469_AT | 388 | −0.01918 | 0.94 | putative CRK1 protein |
| A1ZM056031_AT | 389 | 0.02028 | 1.00 | putative ZF-HD |
| | | | | homeobox protein |
| A1ZM011510_S_AT | 390 | −0.02048 | 1.01 | hypothetical protein |
| A1ZM006707_AT | 391 | 0.01915 | 0.94 | putative oxysterol- |
| | | | | binding protein |
| A1ZM009697_S_AT | 392 | 0.01864 | 0.92 | |
| A1ZM077940_X_AT | 393 | −0.02073 | 1.02 | KIAA0324 protein |
| A1ZM017379_AT | 394 | −0.01795 | 0.88 | putative |
| | | | | phosphohydrolase |
| A1ZM053031_S_AT | 395 | 0.02014 | 0.99 | |
| A1ZM061580_AT | 396 | 0.02170 | 1.07 | DNA topoisomerase III |
| | | | | alpha, putative |
| A1ZM052424_S_AT | 397 | −0.02185 | 1.07 | Peroxisomal fatty acid |
| | | | | beta-oxidation |
| | | | | multifunctional protein |
| | | | | (MFP) |
| A1ZM057110_AT | 398 | 0.01796 | 0.88 | |
| A1ZM000016_S_AT | 399 | −0.01898 | 0.93 | glutathione S- |
| | | | | transferase GST 33 |
| A1ZM043521_AT | 400 | −0.02031 | 1.00 | OSJNBa0063C18.1 |
| A1ZM021664_AT | 401 | −0.02365 | 1.16 | |
| A1ZM057468_AT | 402 | 0.01939 | 0.95 | |
| A1ZM023477_S_AT | 403 | −0.01911 | 0.94 | GCN5L1 family |
| | | | | protein |
| A1ZM012881_AT | 404 | 0.01796 | 0.88 | |
| A1ZM006719_AT | 405 | −0.01807 | 0.89 | P0431H09.18 |
| A1ZM062662_AT | 406 | −0.02339 | 1.15 | putative Cytochrome |
| | | | | P450 |
| A1ZM057238_AT | 407 | −0.01933 | 0.95 | putative PRLI- |
| | | | | interacting factor G |
| A1ZM057856_AT | 408 | −0.01943 | 0.95 | OSJNBb0103I08.17 |
| A1ZM026886_AT | 409 | −0.01896 | 0.93 | unknown |
| A1ZM063309_AT | 410 | −0.02093 | 1.03 | |
| A1ZM006628_A_AT | 411 | −0.01972 | 0.97 | putative zinc finger |
| | | | | protein |
| A1ZM016061_AT | 412 | −0.01801 | 0.88 | voltage-dependent |
| | | | | anion channel protein 2 |
| A1ZM015898_AT | 413 | −0.02228 | 1.09 | P0485G01.21 |
| A1ZM037009_AT | 414 | −0.01858 | 0.91 | unnamed protein |
| | | | | product |
| A1ZM001187_X_AT | 415 | 0.01829 | 0.90 | RING zinc finger |
| | | | | protein-like |
| A1ZM019225_AT | 416 | −0.02393 | 1.18 | |
| A1ZM025982_AT | 417 | 0.02655 | 1.30 | hydrolase, alpha/beta |
| | | | | fold family protein |
| A1ZM085633_AT | 418 | −0.01904 | 0.94 | transducin family |
| | | | | protein/WD-40 repeat |
| | | | | family protein |
| A1ZM043652_S_AT | 419 | 0.02152 | 1.06 | B1144G04.20 |
| A1ZM056158_AT | 420 | −0.02022 | 0.99 | OSJNBa0004G10.17 |
| A1ZM072906_AT | 421 | −0.01944 | 0.95 | putative ER lumen |
| | | | | protein-retaining |
| | | | | receptor |

The regression coefficients for the probesets listed were obtained from transformation of the regression coefficients from the underlying factor model. This factor model found to be most predictive of yield was comprised of just a single factor, suggesting a parsimonious relationship between yield and underlying gene expression variation. That is, the underlying biological relationship may be quite direct.

### Regression of Yield on Markers

The results of PLS regression of yield on SNP markers per se are given in Table 2. The results for markers are remarkably similar to those for probesets. Again, a model with only a single underlying factor was found to be most predictive of yield. Of the total of 653 markers, 24 markers comprised the final model in which variation among the marker genotypes accounted to 48% of the variation in yield.

TABLE 2
Significant markers in partial least squares regression of yield on markers.
Rsquare for the regression model = 48.12.

| | | | | Marker |
|---|---|---|---|---|
| | | | Marker | Regression |
| | | | Regression | Coefficient |
| Marker | Chromosome | Position | Coefficient | VIP |
| NC0173332 | 1 | 33.3 | −0.05546 | 0.97 |
| NC0199555 | 1 | 34.4 | 0.05678 | 0.99 |
| NC0200044 | 1 | 47.5 | −0.05755 | 1.00 |
| NC0173453 | 1 | 172.2 | −0.05473 | 0.95 |
| NC0196949 | 2 | 43.4 | 0.07238 | 1.26 |
| NC0013275 | 2 | 49.7 | 0.06616 | 1.15 |
| NC0199346 | 2 | 50.8 | 0.05537 | 0.96 |
| NC0199297 | 2 | 100.2 | −0.05192 | 0.90 |
| NC0199803 | 3 | 85.6 | 0.05138 | 0.90 |
| NC0173436 | 3 | 86 | 0.06807 | 1.19 |
| NC0000399 | 3 | 121.1 | 0.05894 | 1.03 |
| NC0015965 | 3 | 140.8 | 0.05781 | 1.01 |
| NC0199794 | 3 | 146 | 0.06721 | 1.17 |
| NC0190032 | 3 | 147.8 | 0.05230 | 0.91 |
| NC0199447 | 5 | 80.6 | 0.05728 | 1.00 |
| NC0173900 | 6 | 38.4 | 0.05140 | 0.90 |
| NC0020696 | 6 | 38.5 | −0.05010 | 0.87 |
| NC0199730 | 8 | 79.6 | 0.05528 | 0.96 |
| NC0199735 | 8 | 84.4 | 0.05278 | 0.92 |
| NC0199896 | 8 | 96.4 | −0.05682 | 0.99 |
| NC0033526 | 8 | 153.1 | 0.06435 | 1.12 |
| NC0014545 | 8 | 155.1 | 0.05398 | 0.94 |
| NC0199956 | 8 | 169.9 | −0.05126 | 0.89 |
| NC0199722 | 9 | 79.6 | −0.05027 | 0.88 |

### Probeset Map Positions

Approximate map positions of the 68 probesets most predictive of yield appear in Table 3. The map positions were arrived at by assuming that the probeset locus would be proximal to the marker locus having the lowest p-value for type I error in the single-factor regressions of probesets on markers. If this assumption is valid, precision of placement of the probeset will then be proportional to the size of the corresponding p-value.

TABLE 3
Map positions of markers inferred to be proximal to probesets significantly
associated with yield. The inference of marker proximity is based on the p-value in the
test of single factor regression of probeset expression on markers. The marker with the
most significant p-value in single factor regression of markers on a particular probeset
was inferred to be the marker most closely linked to the probeset.

| | | | | | Proximal |
|---|---|---|---|---|---|
| | | | | | Probeset |
| | | | | Proximal | Marker |
| | | | | Probeset | Single |
| | | Proximal | Proximal | Marker | Factor |
| | Proximal | Probeset | Probeset | Single Factor | Regression |
| | Probeset | Marker | Marker | Regression | Coefficient |
| Probeset | Marker | Chromosome | Position | Coefficient | P-Value |
| A1ZM005469_AT | NC0070876 | 1 | 19.7 | −0.51028 | 0.00000 |
| A1ZM052424_S_AT | NC0173400 | 1 | 32.2 | 0.40073 | 0.00019 |
| A1ZM061580_AT | NC0199555 | 1 | 34.4 | 0.45585 | 0.00002 |
| A1ZM012881_AT | NC0173858 | 1 | 43.8 | −0.37927 | 0.00044 |
| A1ZM021664_AT | NC0200213 | 1 | 67.4 | 0.48218 | 0.00000 |
| A1ZM047966_X_AT | NC0157255 | 1 | 87.7 | −0.56900 | 0.00000 |
| A1ZM055950_S_AT | NC0173484 | 1 | 103.2 | −0.36088 | 0.00087 |
| A1ZM002154_S_AT | NC0199461 | 1 | 176.7 | 0.44649 | 0.00003 |
| A1ZM069545_AT | NC0199461 | 1 | 176.7 | 0.48237 | 0.00000 |
| A1ZM000016_S_AT | NC0173951 | 1 | 229.4 | 0.51887 | 0.00000 |
| A1ZM020483_A_AT | NC0173951 | 1 | 229.4 | 0.45334 | 0.00002 |
| A1ZM063309_AT | NC0173951 | 1 | 229.4 | 0.38945 | 0.00030 |
| A1ZM003299_S_AT | NC0013275 | 2 | 49.7 | 0.47455 | 0.00001 |
| A1ZM061606_X_AT | NC0013275 | 2 | 49.7 | −0.42631 | 0.00007 |
| A1ZM067500_AT | NC0013275 | 2 | 49.7 | −0.48096 | 0.00000 |
| A1ZM079744_AT | NC0013275 | 2 | 49.7 | −0.50613 | 0.00000 |
| A1ZM025769_AT | NC0199346 | 2 | 50.8 | 0.47534 | 0.00001 |
| A1ZM015563_AT | NC0172302 | 2 | 94.0 | −0.60820 | 0.00000 |
| A1ZM033956_AT | NC0174062 | 2 | 129.5 | −0.32410 | 0.00297 |
| A1ZM044683_X_AT | NC0174062 | 2 | 129.5 | 0.56686 | 0.00000 |
| A1ZM049194_AT | NC0033320 | 2 | 173.6 | 0.38122 | 0.00041 |
| A1ZM039148_AT | NC0003536 | 3 | 30.9 | −0.35884 | 0.00093 |
| A1ZM081073_AT | NC0109315 | 3 | 77.4 | −0.38094 | 0.00041 |
| A1ZM025661_AT | NC0173436 | 3 | 86.0 | −0.75890 | 0.00000 |
| A1ZM030937_AT | NC0173436 | 3 | 86.0 | −0.56086 | 0.00000 |
| A1ZM058469_AT | NC0173436 | 3 | 86.0 | −0.72184 | 0.00000 |
| A1ZM056031_AT | NC0199589 | 3 | 91.5 | −0.61791 | 0.00000 |
| A1ZM053031_S_AT | NC0200156 | 3 | 96.1 | −0.33270 | 0.00226 |
| A1ZM011510_S_AT | NC0199741 | 3 | 125.7 | −0.53705 | 0.00000 |
| A1ZM009697_S_AT | NC0015965 | 3 | 140.8 | 0.82543 | 0.00000 |
| A1ZM006707_AT | NC0112924 | 3 | 145.3 | 0.39361 | 0.00025 |
| A1ZM077940_X_AT | NC0193457 | 3 | 216.6 | 0.56649 | 0.00000 |
| A1ZM017379_AT | NC0067577 | 4 | 59.3 | 0.65541 | 0.00000 |
| A1ZM057110_AT | NC0200120 | 5 | 75.4 | −0.50163 | 0.00000 |
| A1ZM033184_AT | NC0200093 | 5 | 131.8 | −0.36687 | 0.00070 |
| A1ZM057468_AT | NC0036529 | 6 | 43.2 | −0.39450 | 0.00024 |
| A1ZM023477_S_AT | NC0028658 | 6 | 52.7 | 0.60562 | 0.00000 |
| A1ZM006719_AT | NC0195223 | 6 | 118.6 | −0.42431 | 0.00007 |
| A1ZM062662_AT | NC0195223 | 6 | 118.6 | −0.33661 | 0.00199 |
| A1ZM057238_AT | NC0199958 | 6 | 142.0 | −0.47570 | 0.00001 |
| A1ZM057856_AT | NC0173859 | 7 | 96.8 | −0.54932 | 0.00000 |
| A1ZM026886_AT | NC0038733 | 7 | 106.5 | 0.31817 | 0.00358 |
| A1ZM045717_AT | NC0200185 | 7 | 147.7 | −0.47190 | 0.00001 |
| A1ZM006628_A_AT | NC0199443 | 8 | 72.9 | −0.55349 | 0.00000 |
| A1ZM016061_AT | NC0194645 | 8 | 76.5 | −0.79465 | 0.00000 |
| A1ZM025982_AT | NC0199730 | 8 | 79.6 | 0.69816 | 0.00000 |
| A1ZM015898_AT | NC0199735 | 8 | 84.4 | −0.48072 | 0.00000 |
| A1ZM037009_AT | NC0199735 | 8 | 84.4 | −0.36918 | 0.00064 |
| A1ZM024567_AT | NC0174094 | 8 | 91.1 | 0.35684 | 0.00100 |
| A1ZM019225_AT | NC0199896 | 8 | 96.4 | 0.45145 | 0.00002 |
| A1ZM085633_AT | NC0199896 | 8 | 96.4 | 0.40512 | 0.00016 |
| A1ZM043652_S_AT | NC0173626 | 8 | 96.8 | 0.47720 | 0.00001 |
| A1ZM056158_AT | NC0173626 | 8 | 96.8 | −0.54652 | 0.00000 |
| A1ZM001187_A_AT | NC0111472 | 8 | 103.3 | −0.53226 | 0.00000 |
| A1ZM001187_X_AT | NC0111472 | 8 | 103.3 | −0.52897 | 0.00000 |
| A1ZM072906_AT | NC0199563 | 8 | 115.6 | 0.33766 | 0.00192 |
| A1ZM004376_S_AT | NC0035106 | 8 | 151.4 | −0.45309 | 0.00002 |
| A1ZM017348_S_AT | NC0033526 | 8 | 153.1 | −0.60888 | 0.00000 |
| A1ZM049860_AT | NC0033526 | 8 | 153.1 | −0.63335 | 0.00000 |
| A1ZM006671_AT | NC0173953 | 9 | 67.4 | 0.63913 | 0.00000 |
| A1ZM032249_AT | NC0173530 | 9 | 78.1 | 0.43519 | 0.00004 |
| A1ZM084532_AT | NC0173530 | 9 | 78.1 | 0.38447 | 0.00036 |
| A1ZM026053_AT | NC0199722 | 9 | 79.6 | −0.52564 | 0.00000 |
| A1ZM031333_AT | NC0199722 | 9 | 79.6 | 0.63064 | 0.00000 |
| A1ZM033611_AT | NC0014240 | 9 | 103.5 | −0.45724 | 0.00002 |
| A1ZM067740_X_AT | NC0014240 | 9 | 103.5 | −0.42137 | 0.00008 |
| A1ZM057725_A_AT | NC0171018 | 10 | 42.7 | 0.55635 | 0.00000 |
| A1ZM043521_AT | NC0199635 | 10 | 106.4 | 0.44964 | 0.00002 |

### Expression QTL

Similarly to the PLS regression of yield on marker genotypes, PLS regression of probeset on marker genotypes was calculated for each of the 68 probesets found to be most predictive of yield. As with yield, the final model for each probeset contained a reduced number of marker loci. The retained marker loci were then assumed to be proximal to an expression QTL (eQTL) for the probeset, facilitating construction of an eQTL map for each probeset (Table 4).

TABLE 4
Map locations of markers proximal to expression QTL of probesets significantly
associated with yield.

| | | Proximal | Proximal | Proximal | Proximal |
|---|---|---|---|---|---|
| | Proximal | Expression | Expression | Expression QTL | Expression QTL |
| | Expression | QTL | QTL | Marker | Marker |
| | QTL | Marker | Marker | Regression | Regression |
| Probeset | Marker | Chromosome | Position | Coefficient | VIP |
| A1ZM000016_S_AT | NC0019524 | 1 | 5.8 | −0.05103 | 0.87 |
| A1ZM000016_S_AT | NC0173400 | 1 | 32.2 | 0.05155 | 0.88 |
| A1ZM000016_S_AT | NC0043266 | 1 | 71.3 | −0.05207 | 0.89 |
| A1ZM000016_S_AT | NC0032745 | 1 | 94.5 | −0.06021 | 1.03 |
| A1ZM000016_S_AT | NC0199760 | 1 | 179.1 | −0.05508 | 0.94 |
| A1ZM000016_S_AT | NC0015937 | 1 | 197.7 | 0.05624 | 0.96 |
| A1ZM000016_S_AT | NC0173395 | 1 | 200.1 | −0.05881 | 1.00 |
| A1ZM000016_S_AT | NC0196534 | 1 | 201.9 | −0.05690 | 0.97 |
| A1ZM000016_S_AT | NC0200158 | 1 | 205.1 | 0.05602 | 0.95 |
| A1ZM000016_S_AT | NC0173951 | 1 | 229.4 | 0.08782 | 1.50 |
| A1ZM000016_S_AT | NC0200048 | 1 | 230.2 | −0.06664 | 1.14 |
| A1ZM000016_S_AT | NC0200138 | 1 | 238.1 | −0.05245 | 0.89 |
| A1ZM000016_S_AT | NC0199954 | 2 | 54.1 | −0.04962 | 0.85 |
| A1ZM000016_S_AT | NC0173900 | 6 | 38.4 | −0.06913 | 1.18 |
| A1ZM000016_S_AT | NC0194397 | 6 | 38.4 | −0.05757 | 0.98 |
| A1ZM000016_S_AT | NC0199422 | 6 | 41.1 | 0.05083 | 0.87 |
| A1ZM000016_S_AT | NC0173500 | 6 | 136.0 | 0.05870 | 1.00 |
| A1ZM000016_S_AT | NC0158250 | 7 | 24.3 | 0.06448 | 1.10 |
| A1ZM000016_S_AT | NC0199331 | 7 | 173.1 | 0.05440 | 0.93 |
| A1ZM000016_S_AT | NC0000129 | 8 | 16.5 | −0.05977 | 1.02 |
| A1ZM000016_S_AT | NC0199387 | 9 | 33.0 | −0.06315 | 1.08 |
| A1ZM000016_S_AT | NC0200107 | 9 | 65.9 | −0.04975 | 0.85 |
| A1ZM000016_S_AT | NC0199722 | 9 | 79.6 | 0.05043 | 0.86 |
| A1ZM000016_S_AT | NC0014240 | 9 | 103.5 | −0.05125 | 0.87 |
| A1ZM000016_S_AT | NC0158122 | 10 | 36.7 | −0.06176 | 1.05 |
| A1ZM000016_S_AT | NC0199810 | 10 | 48.3 | 0.05909 | 1.01 |
| A1ZM000016_S_AT | NC0173818 | 10 | 65.3 | 0.07132 | 1.21 |
| A1ZM000016_S_AT | NC0011002 | 10 | 66.5 | −0.05181 | 0.88 |
| A1ZM001187_A_AT | NC0144205 | 1 | 38.5 | 0.03308 | 1.00 |
| A1ZM001187_A_AT | NC0199461 | 1 | 176.7 | 0.02930 | 0.89 |
| A1ZM001187_A_AT | NC0173402 | 1 | 217.0 | 0.02681 | 0.81 |
| A1ZM001187_A_AT | NC0196949 | 2 | 43.4 | 0.03861 | 1.17 |
| A1ZM001187_A_AT | NC0013275 | 2 | 49.7 | 0.04295 | 1.30 |
| A1ZM001187_A_AT | NC0027262 | 2 | 57.3 | −0.03758 | 1.14 |
| A1ZM001187_A_AT | NC0199350 | 2 | 67.8 | −0.02941 | 0.89 |
| A1ZM001187_A_AT | NC0003725 | 2 | 68.4 | −0.02882 | 0.87 |
| A1ZM001187_A_AT | NC0196659 | 2 | 70.4 | 0.02780 | 0.84 |
| A1ZM001187_A_AT | NC0172302 | 2 | 94.0 | 0.03140 | 0.95 |
| A1ZM001187_A_AT | NC0200126 | 3 | 81.8 | 0.03069 | 0.93 |
| A1ZM001187_A_AT | NC0199803 | 3 | 85.6 | 0.03232 | 0.98 |
| A1ZM001187_A_AT | NC0173436 | 3 | 86.0 | 0.03809 | 1.16 |
| A1ZM001187_A_AT | NC0000399 | 3 | 121.1 | 0.03147 | 0.95 |
| A1ZM001187_A_AT | NC0015965 | 3 | 140.8 | 0.02990 | 0.91 |
| A1ZM001187_A_AT | NC0112924 | 3 | 145.3 | 0.03393 | 1.03 |
| A1ZM001187_A_AT | NC0067577 | 4 | 59.3 | −0.03555 | 1.08 |
| A1ZM001187_A_AT | NC0199812 | 4 | 77.3 | 0.02848 | 0.86 |
| A1ZM001187_A_AT | NC0171755 | 4 | 77.8 | 0.02678 | 0.81 |
| A1ZM001187_A_AT | NC0173414 | 4 | 78.9 | −0.03426 | 1.04 |
| A1ZM001187_A_AT | NC0028257 | 4 | 81.8 | −0.02997 | 0.91 |
| A1ZM001187_A_AT | NC0199680 | 4 | 81.9 | −0.02863 | 0.87 |
| A1ZM001187_A_AT | NC0199410 | 4 | 82.5 | 0.02824 | 0.86 |
| A1ZM001187_A_AT | NC0200151 | 4 | 84.4 | 0.03407 | 1.03 |
| A1ZM001187_A_AT | NC0200083 | 4 | 84.9 | −0.02825 | 0.86 |
| A1ZM001187_A_AT | NC0188018 | 5 | 85.4 | −0.02661 | 0.81 |
| A1ZM001187_A_AT | NC0173452 | 5 | 177.7 | −0.03523 | 1.07 |
| A1ZM001187_A_AT | NC0173900 | 6 | 38.4 | 0.02777 | 0.84 |
| A1ZM001187_A_AT | NC0200165 | 6 | 99.9 | −0.02946 | 0.89 |
| A1ZM001187_A_AT | NC0173876 | 6 | 104.2 | −0.02854 | 0.87 |
| A1ZM001187_A_AT | NC0195223 | 6 | 118.6 | 0.04081 | 1.24 |
| A1ZM001187_A_AT | NC0199409 | 6 | 118.6 | −0.03531 | 1.07 |
| A1ZM001187_A_AT | NC0199958 | 6 | 142.0 | 0.03081 | 0.93 |
| A1ZM001187_A_AT | NC0199339 | 7 | 150.8 | −0.03014 | 0.91 |
| A1ZM001187_A_AT | NC0173490 | 8 | 59.0 | −0.02811 | 0.85 |
| A1ZM001187_A_AT | NC0030011 | 8 | 63.1 | 0.02794 | 0.85 |
| A1ZM001187_A_AT | NC0194645 | 8 | 76.5 | 0.03672 | 1.11 |
| A1ZM001187_A_AT | NC0199730 | 8 | 79.6 | 0.03433 | 1.04 |
| A1ZM001187_A_AT | NC0199735 | 8 | 84.4 | 0.03359 | 1.02 |
| A1ZM001187_A_AT | NC0199770 | 8 | 96.2 | 0.04149 | 1.26 |
| A1ZM001187_A_AT | NC0199896 | 8 | 96.4 | −0.03137 | 0.95 |
| A1ZM001187_A_AT | NC0173626 | 8 | 96.8 | 0.04015 | 1.22 |
| A1ZM001187_A_AT | NC0111472 | 8 | 103.3 | −0.04788 | 1.45 |
| A1ZM001187_A_AT | NC0173350 | 8 | 104.0 | −0.03880 | 1.18 |
| A1ZM001187_A_AT | NC0199563 | 8 | 115.6 | −0.03140 | 0.95 |
| A1ZM001187_A_AT | NC0033526 | 8 | 153.1 | 0.02738 | 0.83 |
| A1ZM001187_X_AT | NC0144205 | 1 | 38.5 | 0.03687 | 1.07 |
| A1ZM001187_X_AT | NC0172493 | 1 | 86.9 | 0.02941 | 0.85 |
| A1ZM001187_X_AT | NC0199461 | 1 | 176.7 | 0.03165 | 0.92 |
| A1ZM001187_X_AT | NC0196949 | 2 | 43.4 | 0.03681 | 1.07 |
| A1ZM001187_X_AT | NC0013275 | 2 | 49.7 | 0.04315 | 1.25 |
| A1ZM001187_X_AT | NC0027262 | 2 | 57.3 | −0.04161 | 1.21 |
| A1ZM001187_X_AT | NC0199350 | 2 | 67.8 | −0.02999 | 0.87 |
| A1ZM001187_X_AT | NC0003725 | 2 | 68.4 | −0.02988 | 0.87 |
| A1ZM001187_X_AT | NC0196659 | 2 | 70.4 | 0.03011 | 0.87 |
| A1ZM001187_X_AT | NC0172302 | 2 | 94.0 | 0.03249 | 0.94 |
| A1ZM001187_X_AT | NC0200126 | 3 | 81.8 | 0.03358 | 0.98 |
| A1ZM001187_X_AT | NC0199803 | 3 | 85.6 | 0.03276 | 0.95 |
| A1ZM001187_X_AT | NC0173436 | 3 | 86.0 | 0.03738 | 1.09 |
| A1ZM001187_X_AT | NC0000399 | 3 | 121.1 | 0.03179 | 0.92 |
| A1ZM001187_X_AT | NC0112924 | 3 | 145.3 | 0.03180 | 0.92 |
| A1ZM001187_X_AT | NC0067577 | 4 | 59.3 | −0.03837 | 1.11 |
| A1ZM001187_X_AT | NC0199812 | 4 | 77.3 | 0.03043 | 0.88 |
| A1ZM001187_X_AT | NC0171755 | 4 | 77.8 | 0.03112 | 0.90 |
| A1ZM001187_X_AT | NC0173414 | 4 | 78.9 | −0.03724 | 1.08 |
| A1ZM001187_X_AT | NC0028257 | 4 | 81.8 | −0.03491 | 1.01 |
| A1ZM001187_X_AT | NC0199680 | 4 | 81.9 | −0.03324 | 0.97 |
| A1ZM001187_X_AT | NC0199410 | 4 | 82.5 | 0.03374 | 0.98 |
| A1ZM001187_X_AT | NC0200151 | 4 | 84.4 | 0.03819 | 1.11 |
| A1ZM001187_X_AT | NC0200083 | 4 | 84.9 | −0.03137 | 0.91 |
| A1ZM001187_X_AT | NC0173929 | 5 | 11.7 | −0.02835 | 0.82 |
| A1ZM001187_X_AT | NC0188018 | 5 | 85.4 | −0.02889 | 0.84 |
| A1ZM001187_X_AT | NC0173452 | 5 | 177.7 | −0.03897 | 1.13 |
| A1ZM001187_X_AT | NC0173876 | 6 | 104.2 | −0.02894 | 0.84 |
| A1ZM001187_X_AT | NC0195223 | 6 | 118.6 | 0.03594 | 1.04 |
| A1ZM001187_X_AT | NC0199409 | 6 | 118.6 | −0.03183 | 0.92 |
| A1ZM001187_X_AT | NC0199958 | 6 | 142.0 | 0.02831 | 0.82 |
| A1ZM001187_X_AT | NC0038733 | 7 | 106.5 | 0.03349 | 0.97 |
| A1ZM001187_X_AT | NC0199339 | 7 | 150.8 | −0.03499 | 1.02 |
| A1ZM001187_X_AT | NC0173490 | 8 | 59.0 | −0.02993 | 0.87 |
| A1ZM001187_X_AT | NC0030011 | 8 | 63.1 | 0.02867 | 0.83 |
| A1ZM001187_X_AT | NC0194645 | 8 | 76.5 | 0.03626 | 1.05 |
| A1ZM001187_X_AT | NC0199730 | 8 | 79.6 | 0.03258 | 0.95 |
| A1ZM001187_X_AT | NC0199735 | 8 | 84.4 | 0.03040 | 0.88 |
| A1ZM001187_X_AT | NC0199770 | 8 | 96.2 | 0.04401 | 1.28 |
| A1ZM001187_X_AT | NC0199896 | 8 | 96.4 | −0.03006 | 0.87 |
| A1ZM001187_X_AT | NC0173626 | 8 | 96.8 | 0.04140 | 1.20 |
| A1ZM001187_X_AT | NC0111472 | 8 | 103.3 | −0.05055 | 1.47 |
| A1ZM001187_X_AT | NC0173350 | 8 | 104.0 | −0.03902 | 1.13 |
| A1ZM001187_X_AT | NC0199563 | 8 | 115.6 | −0.02903 | 0.84 |
| A1ZM002154_S_AT | NC0173334 | 1 | 102.8 | −0.04914 | 0.91 |
| A1ZM002154_S_AT | NC0173484 | 1 | 103.2 | −0.05543 | 1.02 |
| A1ZM002154_S_AT | NC0199391 | 1 | 107.5 | −0.05486 | 1.01 |
| A1ZM002154_S_AT | NC0188455 | 1 | 113.3 | −0.06776 | 1.25 |
| A1ZM002154_S_AT | NC0199986 | 1 | 113.3 | −0.05715 | 1.06 |
| A1ZM002154_S_AT | NC0199461 | 1 | 176.7 | 0.07007 | 1.30 |
| A1ZM002154_S_AT | NC0173951 | 1 | 229.4 | −0.05173 | 0.96 |
| A1ZM002154_S_AT | NC0173409 | 2 | 32.7 | −0.05030 | 0.93 |
| A1ZM002154_S_AT | NC0170324 | 2 | 94.0 | 0.04976 | 0.92 |
| A1ZM002154_S_AT | NC0174027 | 2 | 99.2 | −0.05375 | 0.99 |
| A1ZM002154_S_AT | NC0199297 | 2 | 100.2 | −0.05008 | 0.93 |
| A1ZM002154_S_AT | NC0199851 | 2 | 165.2 | −0.04933 | 0.91 |
| A1ZM002154_S_AT | NC0033320 | 2 | 173.6 | 0.05233 | 0.97 |
| A1ZM002154_S_AT | NC0051616 | 3 | 40.4 | −0.05721 | 1.06 |
| A1ZM002154_S_AT | NC0199522 | 3 | 90.3 | 0.05926 | 1.10 |
| A1ZM002154_S_AT | NC0112481 | 3 | 183.6 | −0.06465 | 1.20 |
| A1ZM002154_S_AT | NC0170468 | 3 | 216.7 | 0.04604 | 0.85 |
| A1ZM002154_S_AT | NC0036637 | 5 | 100.0 | −0.04879 | 0.90 |
| A1ZM002154_S_AT | NC0196774 | 6 | 69.4 | 0.05249 | 0.97 |
| A1ZM002154_S_AT | NC0173525 | 6 | 70.2 | 0.05110 | 0.94 |
| A1ZM002154_S_AT | NC0199800 | 7 | 114.5 | −0.05174 | 0.96 |
| A1ZM002154_S_AT | NC0199443 | 8 | 72.9 | 0.06171 | 1.14 |
| A1ZM002154_S_AT | NC0194645 | 8 | 76.5 | 0.05797 | 1.07 |
| A1ZM002154_S_AT | NC0199730 | 8 | 79.6 | 0.05532 | 1.02 |
| A1ZM002154_S_AT | NC0199735 | 8 | 84.4 | 0.05286 | 0.98 |
| A1ZM002154_S_AT | NC0174094 | 8 | 91.1 | 0.04634 | 0.86 |
| A1ZM002154_S_AT | NC0199563 | 8 | 115.6 | −0.04742 | 0.88 |
| A1ZM002154_S_AT | NC0033526 | 8 | 153.1 | 0.04597 | 0.85 |
| A1ZM002154_S_AT | NC0014545 | 8 | 155.1 | 0.04626 | 0.86 |
| A1ZM002154_S_AT | NC0107256 | 9 | 68.5 | 0.05121 | 0.95 |
| A1ZM002154_S_AT | NC0173571 | 9 | 90.4 | −0.05597 | 1.03 |
| A1ZM002154_S_AT | NC0148189 | 9 | 91.0 | −0.05635 | 1.04 |
| A1ZM003299_S_AT | NC0199461 | 1 | 176.7 | 0.04694 | 1.00 |
| A1ZM003299_S_AT | NC0173409 | 2 | 32.7 | −0.04268 | 0.91 |
| A1ZM003299_S_AT | NC0196949 | 2 | 43.4 | 0.05657 | 1.20 |
| A1ZM003299_S_AT | NC0013275 | 2 | 49.7 | 0.06505 | 1.38 |
| A1ZM003299_S_AT | NC0199346 | 2 | 50.8 | 0.04034 | 0.86 |
| A1ZM003299_S_AT | NC0002630 | 2 | 54.1 | −0.04810 | 1.02 |
| A1ZM003299_S_AT | NC0027262 | 2 | 57.3 | −0.04482 | 0.95 |
| A1ZM003299_S_AT | NC0015965 | 3 | 140.8 | 0.05244 | 1.11 |
| A1ZM003299_S_AT | NC0199794 | 3 | 146.0 | 0.05729 | 1.22 |
| A1ZM003299_S_AT | NC0171755 | 4 | 77.8 | 0.04259 | 0.91 |
| A1ZM003299_S_AT | NC0199680 | 4 | 81.9 | −0.04290 | 0.91 |
| A1ZM003299_S_AT | NC0199410 | 4 | 82.5 | 0.04138 | 0.88 |
| A1ZM003299_S_AT | NC0199832 | 4 | 84.7 | 0.04994 | 1.06 |
| A1ZM003299_S_AT | NC0200083 | 4 | 84.9 | −0.04708 | 1.00 |
| A1ZM003299_S_AT | NC0199826 | 4 | 190.8 | 0.04467 | 0.95 |
| A1ZM003299_S_AT | NC0199455 | 6 | 73.3 | 0.04219 | 0.90 |
| A1ZM003299_S_AT | NC0173962 | 6 | 96.7 | 0.04980 | 1.06 |
| A1ZM003299_S_AT | NC0194601 | 6 | 143.7 | −0.04979 | 1.06 |
| A1ZM003299_S_AT | NC0199685 | 7 | 28.6 | 0.04194 | 0.89 |
| A1ZM003299_S_AT | NC0199937 | 7 | 31.2 | 0.04587 | 0.98 |
| A1ZM003299_S_AT | NC0174167 | 7 | 58.0 | −0.04487 | 0.95 |
| A1ZM003299_S_AT | NC0199770 | 8 | 96.2 | 0.04193 | 0.89 |
| A1ZM003299_S_AT | NC0173626 | 8 | 96.8 | 0.04549 | 0.97 |
| A1ZM003299_S_AT | NC0111472 | 8 | 103.3 | −0.04147 | 0.88 |
| A1ZM003299_S_AT | NC0199722 | 9 | 79.6 | −0.04289 | 0.91 |
| A1ZM003299_S_AT | NC0148189 | 9 | 91.0 | −0.04498 | 0.96 |
| A1ZM004376_S_AT | NC0173334 | 1 | 102.8 | −0.05260 | 0.92 |
| A1ZM004376_S_AT | NC0173484 | 1 | 103.2 | −0.05077 | 0.89 |
| A1ZM004376_S_AT | NC0199986 | 1 | 113.3 | −0.05762 | 1.01 |
| A1ZM004376_S_AT | NC0199350 | 2 | 67.8 | −0.05746 | 1.00 |
| A1ZM004376_S_AT | NC0196659 | 2 | 70.4 | 0.05178 | 0.90 |
| A1ZM004376_S_AT | NC0000399 | 3 | 121.1 | 0.05486 | 0.96 |
| A1ZM004376_S_AT | NC0190032 | 3 | 147.8 | 0.05218 | 0.91 |
| A1ZM004376_S_AT | NC0018073 | 3 | 201.6 | 0.05056 | 0.88 |
| A1ZM004376_S_AT | NC0199978 | 4 | 70.6 | 0.06506 | 1.13 |
| A1ZM004376_S_AT | NC0199812 | 4 | 77.3 | 0.06070 | 1.06 |
| A1ZM004376_S_AT | NC0171755 | 4 | 77.8 | 0.05270 | 0.92 |
| A1ZM004376_S_AT | NC0173414 | 4 | 78.9 | −0.04993 | 0.87 |
| A1ZM004376_S_AT | NC0199603 | 4 | 79.8 | −0.05651 | 0.99 |
| A1ZM004376_S_AT | NC0200083 | 4 | 84.9 | −0.05731 | 1.00 |
| A1ZM004376_S_AT | NC0189286 | 4 | 177.0 | 0.05243 | 0.91 |
| A1ZM004376_S_AT | NC0147215 | 4 | 179.6 | −0.06321 | 1.10 |
| A1ZM004376_S_AT | NC0194601 | 6 | 143.7 | −0.06049 | 1.06 |
| A1ZM004376_S_AT | NC0173584 | 7 | 8.5 | −0.05920 | 1.03 |
| A1ZM004376_S_AT | NC0174551 | 7 | 11.2 | 0.05404 | 0.94 |
| A1ZM004376_S_AT | NC0199805 | 8 | 51.7 | 0.05602 | 0.98 |
| A1ZM004376_S_AT | NC0199563 | 8 | 115.6 | −0.05861 | 1.02 |
| A1ZM004376_S_AT | NC0035106 | 8 | 151.4 | −0.07551 | 1.32 |
| A1ZM004376_S_AT | NC0033526 | 8 | 153.1 | 0.05998 | 1.05 |
| A1ZM004376_S_AT | NC0014545 | 8 | 155.1 | 0.06660 | 1.16 |
| A1ZM004376_S_AT | NC0199880 | 8 | 161.8 | 0.04940 | 0.86 |
| A1ZM005469_AT | NC0039304 | 1 | 19.7 | −0.06679 | 0.99 |
| A1ZM005469_AT | NC0070876 | 1 | 19.7 | −0.09737 | 1.45 |
| A1ZM005469_AT | NC0173555 | 1 | 22.9 | 0.09283 | 1.38 |
| A1ZM005469_AT | NC0173332 | 1 | 33.3 | −0.06261 | 0.93 |
| A1ZM005469_AT | NC0157397 | 1 | 87.7 | 0.05918 | 0.88 |
| A1ZM005469_AT | NC0171154 | 1 | 88.1 | −0.05657 | 0.84 |
| A1ZM005469_AT | NC0199317 | 2 | 138.2 | −0.06102 | 0.91 |
| A1ZM005469_AT | NC0003536 | 3 | 30.9 | 0.06132 | 0.91 |
| A1ZM005469_AT | NC0010671 | 4 | 63.7 | −0.05782 | 0.86 |
| A1ZM005469_AT | NC0173568 | 4 | 150.5 | −0.06954 | 1.03 |
| A1ZM005469_AT | NC0187978 | 7 | 165.7 | −0.06223 | 0.92 |
| A1ZM005469_AT | NC0199331 | 7 | 173.1 | −0.05680 | 0.84 |
| A1ZM005469_AT | NC0199735 | 8 | 84.4 | 0.06557 | 0.97 |
| A1ZM005469_AT | NC0199440 | 8 | 87.9 | −0.07186 | 1.07 |
| A1ZM005469_AT | NC0199313 | 8 | 89.3 | 0.07323 | 1.09 |
| A1ZM005469_AT | NC0199860 | 8 | 93.9 | 0.06022 | 0.89 |
| A1ZM005469_AT | NC0199896 | 8 | 96.4 | −0.07520 | 1.12 |
| A1ZM005469_AT | NC0199563 | 8 | 115.6 | −0.05771 | 0.86 |
| A1ZM005469_AT | NC0199525 | 9 | 90.9 | −0.05799 | 0.86 |
| A1ZM005469_AT | NC0148189 | 9 | 91.0 | −0.06203 | 0.92 |
| A1ZM006628_A_AT | NC0035426 | 1 | 34.5 | 0.03411 | 0.86 |
| A1ZM006628_A_AT | NC0170811 | 1 | 153.9 | 0.04197 | 1.06 |
| A1ZM006628_A_AT | NC0173449 | 1 | 154.4 | −0.04329 | 1.10 |
| A1ZM006628_A_AT | NC0009148 | 1 | 213.0 | −0.03363 | 0.85 |
| A1ZM006628_A_AT | NC0112831 | 2 | 9.0 | 0.03265 | 0.83 |
| A1ZM006628_A_AT | NC0199954 | 2 | 54.1 | −0.03498 | 0.88 |
| A1ZM006628_A_AT | NC0200081 | 3 | 115.9 | −0.04317 | 1.09 |
| A1ZM006628_A_AT | NC0112924 | 3 | 145.3 | −0.03240 | 0.82 |
| A1ZM006628_A_AT | NC0112481 | 3 | 183.6 | 0.03312 | 0.84 |
| A1ZM006628_A_AT | NC0199293 | 5 | 62.9 | −0.04181 | 1.06 |
| A1ZM006628_A_AT | NC0173716 | 5 | 81.5 | −0.03877 | 0.98 |
| A1ZM006628_A_AT | NC0194693 | 5 | 89.9 | −0.04439 | 1.12 |
| A1ZM006628_A_AT | NC0078090 | 5 | 141.8 | −0.03512 | 0.89 |
| A1ZM006628_A_AT | NC0173396 | 5 | 143.1 | −0.03442 | 0.87 |
| A1ZM006628_A_AT | NC0042059 | 5 | 143.2 | −0.03846 | 0.97 |
| A1ZM006628_A_AT | NC0173843 | 5 | 143.2 | 0.03531 | 0.89 |
| A1ZM006628_A_AT | NC0020696 | 6 | 38.5 | 0.03686 | 0.93 |
| A1ZM006628_A_AT | NC0199750 | 6 | 81.6 | 0.03475 | 0.88 |
| A1ZM006628_A_AT | NC0013445 | 6 | 131.2 | −0.03456 | 0.87 |
| A1ZM006628_A_AT | NC0199331 | 7 | 173.1 | 0.03451 | 0.87 |
| A1ZM006628_A_AT | NC0079084 | 8 | 65.2 | 0.04295 | 1.09 |
| A1ZM006628_A_AT | NC0200128 | 8 | 65.7 | −0.04161 | 1.05 |
| A1ZM006628_A_AT | NC0171070 | 8 | 70.0 | −0.04686 | 1.19 |
| A1ZM006628_A_AT | NC0173531 | 8 | 70.0 | 0.03235 | 0.82 |
| A1ZM006628_A_AT | NC0199443 | 8 | 72.9 | −0.05684 | 1.44 |
| A1ZM006628_A_AT | NC0194645 | 8 | 76.5 | −0.04259 | 1.08 |
| A1ZM006628_A_AT | NC0199730 | 8 | 79.6 | −0.04894 | 1.24 |
| A1ZM006628_A_AT | NC0199735 | 8 | 84.4 | −0.03664 | 0.93 |
| A1ZM006628_A_AT | NC0199770 | 8 | 96.2 | −0.03635 | 0.92 |
| A1ZM006628_A_AT | NC0199896 | 8 | 96.4 | 0.05208 | 1.32 |
| A1ZM006628_A_AT | NC0173626 | 8 | 96.8 | −0.04195 | 1.06 |
| A1ZM006628_A_AT | NC0111472 | 8 | 103.3 | 0.03729 | 0.94 |
| A1ZM006628_A_AT | NC003S106 | 8 | 151.4 | 0.03548 | 0.90 |
| A1ZM006628_A_AT | NC0033526 | 8 | 153.1 | −0.03808 | 0.96 |
| A1ZM006628_A_AT | NC0199956 | 8 | 169.9 | 0.04077 | 1.03 |
| A1ZM006671_AT | NC0029744 | 9 | 67.4 | −0.16112 | 1.13 |
| A1ZM006671_AT | NC0173953 | 9 | 67.4 | 0.16462 | 1.15 |
| A1ZM006671_AT | NC0020872 | 9 | 76.3 | 0.13487 | 0.94 |
| A1ZM006671_AT | NC0173530 | 9 | 78.1 | −0.12930 | 0.90 |
| A1ZM006671_AT | NC0199722 | 9 | 79.6 | −0.11967 | 0.84 |
| A1ZM006707_AT | NC0199703 | 1 | 26.9 | −0.05681 | 0.95 |
| A1ZM006707_AT | NC0029053 | 1 | 88.1 | −0.06064 | 1.02 |
| A1ZM006707_AT | NC0199461 | 1 | 176.7 | 0.05488 | 0.92 |
| A1ZM006707_AT | NC0013275 | 2 | 49.7 | 0.06825 | 1.15 |
| A1ZM006707_AT | NC0199346 | 2 | 50.8 | 0.05387 | 0.91 |
| A1ZM006707_AT | NC0002630 | 2 | 54.1 | −0.05599 | 0.94 |
| A1ZM006707_AT | NC0199803 | 3 | 85.6 | 0.05449 | 0.92 |
| A1ZM006707_AT | NC0009470 | 3 | 88.0 | −0.05683 | 0.96 |
| A1ZM006707_AT | NC0199522 | 3 | 90.3 | 0.05701 | 0.96 |
| A1ZM006707_AT | NC0112924 | 3 | 145.3 | 0.07244 | 1.22 |
| A1ZM006707_AT | NC0199826 | 4 | 190.8 | 0.05399 | 0.91 |
| A1ZM006707_AT | NC0200106 | 5 | 124.4 | 0.05704 | 0.96 |
| A1ZM006707_AT | NC0107877 | 5 | 132.7 | −0.06609 | 1.11 |
| A1ZM006707_AT | NC0200064 | 7 | 65.3 | −0.05462 | 0.92 |
| A1ZM006707_AT | NC0000129 | 8 | 16.5 | 0.05852 | 0.98 |
| A1ZM006707_AT | NC0173490 | 8 | 59.0 | −0.05517 | 0.93 |
| A1ZM006707_AT | NC0199735 | 8 | 84.4 | 0.05578 | 0.94 |
| A1ZM006707_AT | NC0199770 | 8 | 96.2 | 0.06662 | 1.12 |
| A1ZM006707_AT | NC0173626 | 8 | 96.8 | 0.05897 | 0.99 |
| A1ZM006707_AT | NC0111472 | 8 | 103.3 | −0.06684 | 1.12 |
| A1ZM006707_AT | NC0199563 | 8 | 115.6 | −0.05409 | 0.91 |
| A1ZM006707_AT | NC0020872 | 9 | 76.3 | 0.05629 | 0.95 |
| A1ZM006707_AT | NC0199722 | 9 | 79.6 | −0.06717 | 1.13 |
| A1ZM006719_AT | NC0039304 | 1 | 19.7 | 0.05018 | 0.97 |
| A1ZM006719_AT | NC0199391 | 1 | 107.5 | 0.05663 | 1.09 |
| A1ZM006719_AT | NC0188455 | 1 | 113.3 | 0.05374 | 1.04 |
| A1ZM006719_AT | NC0199621 | 1 | 133.4 | −0.04793 | 0.93 |
| A1ZM006719_AT | NC0170811 | 1 | 153.9 | 0.04703 | 0.91 |
| A1ZM006719_AT | NC0173449 | 1 | 154.4 | −0.05265 | 1.02 |
| A1ZM006719_AT | NC0199461 | 1 | 176.7 | −0.05192 | 1.00 |
| A1ZM006719_AT | NC0200048 | 1 | 230.2 | −0.04612 | 0.89 |
| A1ZM006719_AT | NC0013275 | 2 | 49.7 | −0.05838 | 1.13 |
| A1ZM006719_AT | NC0199310 | 2 | 75.7 | −0.04636 | 0.90 |
| A1ZM006719_AT | NC0198241 | 3 | 102.0 | −0.04665 | 0.90 |
| A1ZM006719_AT | NC0187941 | 4 | 121.0 | 0.04796 | 0.93 |
| A1ZM006719_AT | NC0200171 | 5 | 73.2 | −0.04515 | 0.87 |
| A1ZM006719_AT | NC0056874 | 5 | 73.5 | 0.04842 | 0.94 |
| A1ZM006719_AT | NC0199425 | 5 | 81.2 | −0.04683 | 0.90 |
| A1ZM006719_AT | NC0033924 | 6 | 36.2 | −0.04758 | 0.92 |
| A1ZM006719_AT | NC0042917 | 6 | 36.4 | −0.04530 | 0.88 |
| A1ZM006719_AT | NC0195223 | 6 | 118.6 | −0.06522 | 1.26 |
| A1ZM006719_AT | NC0199409 | 6 | 118.6 | 0.05817 | 1.12 |
| A1ZM006719_AT | NC0200199 | 7 | 12.4 | −0.04832 | 0.93 |
| A1ZM006719_AT | NC0199685 | 7 | 28.6 | −0.05863 | 1.13 |
| A1ZM006719_AT | NC0199937 | 7 | 31.2 | −0.05052 | 0.98 |
| A1ZM006719_AT | NC0199889 | 7 | 99.4 | −0.05166 | 1.00 |
| A1ZM006719_AT | NC0199735 | 8 | 84.4 | −0.05371 | 1.04 |
| A1ZM006719_AT | NC0199770 | 8 | 96.2 | −0.05053 | 0.98 |
| A1ZM006719_AT | NC0173626 | 8 | 96.8 | −0.06013 | 1.16 |
| A1ZM006719_AT | NC0111472 | 8 | 103.3 | 0.05448 | 1.05 |
| A1ZM006719_AT | NC0035106 | 8 | 151.4 | 0.06024 | 1.16 |
| A1ZM006719_AT | NC0033526 | 8 | 153.1 | −0.04659 | 0.90 |
| A1ZM006719_AT | NC0011002 | 10 | 66.5 | −0.04751 | 0.92 |
| A1ZM009697_S_AT | NC0015965 | 3 | 140.8 | 0.32642 | 1.02 |
| A1ZM009697_S_AT | NC0199794 | 3 | 146.0 | 0.32244 | 1.01 |
| A1ZM009697_S_AT | NC0190032 | 3 | 147.8 | 0.31129 | 0.97 |
| A1ZM011510_S_AT | NC0036726 | 1 | 35.2 | −0.03738 | 0.85 |
| A1ZM011510_S_AT | NC0199491 | 1 | 183.9 | 0.03616 | 0.82 |
| A1ZM011510_S_AT | NC0196949 | 2 | 43.4 | −0.05180 | 1.18 |
| A1ZM011510_S_AT | NC0013275 | 2 | 49.7 | −0.04918 | 1.12 |
| A1ZM011510_S_AT | NC0027262 | 2 | 57.3 | 0.03790 | 0.86 |
| A1ZM011510_S_AT | NC0173436 | 3 | 86.0 | −0.03940 | 0.90 |
| A1ZM011510_S_AT | NC0000399 | 3 | 121.1 | −0.04140 | 0.94 |
| A1ZM011510_S_AT | NC0199741 | 3 | 125.7 | −0.06208 | 1.41 |
| A1ZM011510_S_AT | NC0112924 | 3 | 145.3 | −0.04940 | 1.12 |
| A1ZM011510_S_AT | NC0199794 | 3 | 146.0 | −0.03949 | 0.90 |
| A1ZM011510_S_AT | NC0000189 | 3 | 215.3 | 0.03530 | 0.80 |
| A1ZM011510_S_AT | NC0193457 | 3 | 216.6 | 0.03651 | 0.83 |
| A1ZM011510_S_AT | NC0024368 | 4 | 162.6 | −0.03711 | 0.84 |
| A1ZM011510_S_AT | NC0195223 | 6 | 118.6 | −0.04211 | 0.96 |
| A1ZM011510_S_AT | NC0199409 | 6 | 118.6 | 0.03745 | 0.85 |
| A1ZM011510_S_AT | NC0194645 | 8 | 76.5 | −0.04861 | 1.11 |
| A1ZM011510_S_AT | NC0199730 | 8 | 79.6 | −0.05117 | 1.16 |
| A1ZM011510_S_AT | NC0199770 | 8 | 96.2 | −0.04038 | 0.92 |
| A1ZM011510_S_AT | NC0199896 | 8 | 96.4 | 0.03841 | 0.87 |
| A1ZM011510_S_AT | NC0111472 | 8 | 103.3 | 0.03984 | 0.91 |
| A1ZM011510_S_AT | NC0035106 | 8 | 151.4 | 0.04193 | 0.95 |
| A1ZM011510_S_AT | NC0029744 | 9 | 67.4 | 0.04876 | 1.11 |
| A1ZM011510_S_AT | NC0173953 | 9 | 67.4 | −0.04219 | 0.96 |
| A1ZM011510_S_AT | NC0173530 | 9 | 78.1 | 0.03813 | 0.87 |
| A1ZM011510_S_AT | NC0199722 | 9 | 79.6 | 0.04372 | 0.99 |
| A1ZM011510_S_AT | NC0030512 | 9 | 103.6 | −0.03620 | 0.82 |
| A1ZM011510_S_AT | NC0200144 | 9 | 110.4 | 0.05930 | 1.35 |
| A1ZM011510_S_AT | NC0051922 | 9 | 110.5 | 0.04828 | 1.10 |
| A1ZM011510_S_AT | NC0029024 | 9 | 116.9 | 0.04991 | 1.14 |
| A1ZM011510_S_AT | NC0199898 | 9 | 119.2 | 0.04984 | 1.13 |
| A1ZM011510_S_AT | NC0199686 | 10 | 49.1 | −0.03610 | 0.82 |
| A1ZM011510_S_AT | NC0199366 | 10 | 51.6 | −0.03634 | 0.83 |
| A1ZM011510_S_AT | NC0053108 | 10 | 61.2 | −0.04216 | 0.96 |
| A1ZM011510_S_AT | NC0011002 | 10 | 66.5 | −0.05247 | 1.19 |
| A1ZM012881_AT | NC0173858 | 1 | 43.8 | −0.11769 | 1.12 |
| A1ZM012881_AT | NC0199415 | 2 | 84.7 | −0.10719 | 1.02 |
| A1ZM012881_AT | NC0200109 | 2 | 95.1 | 0.10521 | 1.00 |
| A1ZM012881_AT | NC0157517 | 2 | 99.0 | −0.09214 | 0.88 |
| A1ZM012881_AT | NC0199535 | 6 | 75.4 | −0.09347 | 0.89 |
| A1ZM012881_AT | NC0199750 | 6 | 81.6 | 0.10202 | 0.97 |
| A1ZM012881_AT | NC0173962 | 6 | 96.7 | 0.11313 | 1.08 |
| A1ZM012881_AT | NC0199480 | 6 | 99.9 | −0.11309 | 1.07 |
| A1ZM012881_AT | NC0173357 | 7 | 131.0 | −0.10145 | 0.96 |
| A1ZM012881_AT | NC0145457 | 8 | 91.1 | −0.09398 | 0.89 |
| A1ZM012881_AT | NC0174094 | 8 | 91.1 | 0.11358 | 1.08 |
| A1ZM012881_AT | NC0148189 | 9 | 91.0 | −0.10564 | 1.00 |
| A1ZM015563_AT | NC0013275 | 2 | 49.7 | −0.07268 | 1.11 |
| A1ZM015563_AT | NC0199346 | 2 | 50.8 | −0.05930 | 0.91 |
| A1ZM015563_AT | NC0002630 | 2 | 54.1 | 0.06350 | 0.97 |
| A1ZM015563_AT | NC0199954 | 2 | 54.1 | −0.06903 | 1.06 |
| A1ZM015563_AT | NC0170324 | 2 | 94.0 | −0.07870 | 1.21 |
| A1ZM015563_AT | NC0172302 | 2 | 94.0 | −0.08430 | 1.29 |
| A1ZM015563_AT | NC0000399 | 3 | 121.1 | −0.05942 | 0.91 |
| A1ZM015563_AT | NC0112924 | 3 | 145.3 | −0.07793 | 1.19 |
| A1ZM015563_AT | NC0199293 | 5 | 62.9 | −0.05563 | 0.85 |
| A1ZM015563_AT | NC0194693 | 5 | 89.9 | −0.07535 | 1.15 |
| A1ZM015563_AT | NC0187978 | 7 | 165.7 | 0.06145 | 0.94 |
| A1ZM015563_AT | NC0199443 | 8 | 72.9 | −0.05399 | 0.83 |
| A1ZM015563_AT | NC0194645 | 8 | 76.5 | −0.06615 | 1.01 |
| A1ZM015563_AT | NC0199730 | 8 | 79.6 | −0.06509 | 1.00 |
| A1ZM015563_AT | NC0199735 | 8 | 84.4 | −0.06457 | 0.99 |
| A1ZM015563_AT | NC0199896 | 8 | 96.4 | 0.06627 | 1.02 |
| A1ZM015563_AT | NC0111472 | 8 | 103.3 | 0.05471 | 0.84 |
| A1ZM015563_AT | NC0199563 | 8 | 115.6 | 0.06139 | 0.94 |
| A1ZM015563_AT | NC0035106 | 8 | 151.4 | 0.05250 | 0.80 |
| A1ZM015563_AT | NC0033526 | 8 | 153.1 | −0.06321 | 0.97 |
| A1ZM015563_AT | NC0014545 | 8 | 155.1 | −0.05266 | 0.81 |
| A1ZM015898_AT | NC0029053 | 1 | 88.1 | 0.05599 | 0.84 |
| A1ZM015898_AT | NC0013275 | 2 | 49.7 | −0.06396 | 0.96 |
| A1ZM015898_AT | NC0199346 | 2 | 50.8 | −0.06045 | 0.90 |
| A1ZM015898_AT | NC0199480 | 6 | 99.9 | 0.05898 | 0.88 |
| A1ZM015898_AT | NC0199339 | 7 | 150.8 | 0.06161 | 0.92 |
| A1ZM015898_AT | NC0199730 | 8 | 79.6 | −0.05413 | 0.81 |
| A1ZM015898_AT | NC0199735 | 8 | 84.4 | −0.08786 | 1.31 |
| A1ZM015898_AT | NC0199440 | 8 | 87.9 | 0.06348 | 0.95 |
| A1ZM015898_AT | NC0199313 | 8 | 89.3 | −0.05530 | 0.83 |
| A1ZM015898_AT | NC0145457 | 8 | 91.1 | 0.06383 | 0.95 |
| A1ZM015898_AT | NC0199860 | 8 | 93.9 | −0.06709 | 1.00 |
| A1ZM015898_AT | NC0151542 | 8 | 96.2 | 0.06277 | 0.94 |
| A1ZM015898_AT | NC0199770 | 8 | 96.2 | −0.08183 | 1.22 |
| A1ZM015898_AT | NC0199896 | 8 | 96.4 | 0.06821 | 1.02 |
| A1ZM015898_AT | NC0173626 | 8 | 96.8 | −0.08382 | 1.25 |
| A1ZM015898_AT | NC0111472 | 8 | 103.3 | 0.06265 | 0.94 |
| A1ZM015898_AT | NC0157736 | 9 | 56.6 | 0.06973 | 1.04 |
| A1ZM015898_AT | NC0200122 | 9 | 56.6 | −0.06973 | 1.04 |
| A1ZM016061_AT | NC0170324 | 2 | 94.0 | −0.10967 | 0.90 |
| A1ZM016061_AT | NC0172302 | 2 | 94.0 | −0.11856 | 0.97 |
| A1ZM016061_AT | NC0200126 | 3 | 81.8 | −0.10494 | 0.86 |
| A1ZM016061_AT | NC0112924 | 3 | 145.3 | −0.11330 | 0.93 |
| A1ZM016061_AT | NC0199443 | 8 | 72.9 | −0.12072 | 0.99 |
| A1ZM016061_AT | NC0194645 | 8 | 76.5 | −0.14993 | 1.23 |
| A1ZM016061_AT | NC0199730 | 8 | 79.6 | −0.15436 | 1.27 |
| A1ZM016061_AT | NC0199735 | 8 | 84.4 | −0.10990 | 0.90 |
| A1ZM016061_AT | NC0199896 | 8 | 96.4 | 0.12567 | 1.03 |
| A1ZM016061_AT | NC0111472 | 8 | 103.3 | 0.09971 | 0.82 |
| A1ZM017348_S_AT | NC0199461 | 1 | 176.7 | −0.04076 | 0.99 |
| A1ZM017348_S_AT | NC0199760 | 1 | 179.1 | −0.03946 | 0.96 |
| A1ZM017348_S_AT | NC0200153 | 1 | 182.8 | 0.03759 | 0.91 |
| A1ZM017348_S_AT | NC0080029 | 2 | 33.1 | −0.03741 | 0.91 |
| A1ZM017348_S_AT | NC0196949 | 2 | 43.4 | −0.04274 | 1.04 |
| A1ZM017348_S_AT | NC0013275 | 2 | 49.7 | −0.03625 | 0.88 |
| A1ZM017348_S_AT | NC0199350 | 2 | 67.8 | 0.03362 | 0.82 |
| A1ZM017348_S_AT | NC0200081 | 3 | 115.9 | −0.03753 | 0.91 |
| A1ZM017348_S_AT | NC0000399 | 3 | 121.1 | −0.05109 | 1.24 |
| A1ZM017348_S_AT | NC0199741 | 3 | 125.7 | −0.04216 | 1.02 |
| A1ZM017348_S_AT | NC0015965 | 3 | 140.8 | −0.03767 | 0.92 |
| A1ZM017348_S_AT | NC0112924 | 3 | 145.3 | −0.04686 | 1.14 |
| A1ZM017348_S_AT | NC0199794 | 3 | 146.0 | −0.03333 | 0.81 |
| A1ZM017348_S_AT | NC0190032 | 3 | 147.8 | −0.03545 | 0.86 |
| A1ZM017348_S_AT | NC0199913 | 3 | 152.8 | −0.03557 | 0.86 |
| A1ZM017348_S_AT | NC0035568 | 3 | 166.8 | 0.03586 | 0.87 |
| A1ZM017348_S_AT | NC0174066 | 3 | 168.4 | 0.03532 | 0.86 |
| A1ZM017348_S_AT | NC0199675 | 5 | 45.9 | −0.03563 | 0.87 |
| A1ZM017348_S_AT | NC0199293 | 5 | 62.9 | −0.04994 | 1.21 |
| A1ZM017348_S_AT | NC0199447 | 5 | 80.6 | −0.05312 | 1.29 |
| A1ZM017348_S_AT | NC0173900 | 6 | 38.4 | −0.03339 | 0.81 |
| A1ZM017348_S_AT | NC0020696 | 6 | 38.5 | 0.03766 | 0.92 |
| A1ZM017348_S_AT | NC0029612 | 6 | 57.8 | −0.03349 | 0.81 |
| A1ZM017348_S_AT | NC0200165 | 6 | 99.9 | 0.03400 | 0.83 |
| A1ZM017348_S_AT | NC0195223 | 6 | 118.6 | −0.03929 | 0.96 |
| A1ZM017348_S_AT | NC0199409 | 6 | 118.6 | 0.04302 | 1.05 |
| A1ZM017348_S_AT | NC0199331 | 7 | 173.1 | 0.03932 | 0.96 |
| A1ZM017348_S_AT | NC0199443 | 8 | 72.9 | −0.03470 | 0.84 |
| A1ZM017348_S_AT | NC0199730 | 8 | 79.6 | −0.03911 | 0.95 |
| A1ZM017348_S_AT | NC0199896 | 8 | 96.4 | 0.03404 | 0.83 |
| A1ZM017348_S_AT | NC0035106 | 8 | 151.4 | 0.05669 | 1.38 |
| A1ZM017348_S_AT | NC0033526 | 8 | 153.1 | −0.06457 | 1.57 |
| A1ZM017348_S_AT | NC0014545 | 8 | 155.1 | −0.05188 | 1.26 |
| A1ZM017348_S_AT | NC0199956 | 8 | 169.9 | 0.04475 | 1.09 |
| A1ZM017348_S_AT | NC0056940 | 9 | 0.4 | 0.03748 | 0.91 |
| A1ZM017348_S_AT | NC0157951 | 10 | 48.3 | 0.03676 | 0.89 |
| A1ZM017379_AT | NC0144205 | 1 | 38.5 | −0.06592 | 0.91 |
| A1ZM017379_AT | NC0200169 | 1 | 159.2 | 0.06876 | 0.95 |
| A1ZM017379_AT | NC0199929 | 2 | 27.0 | 0.06761 | 0.93 |
| A1ZM017379_AT | NC0023415 | 2 | 67.3 | −0.07125 | 0.98 |
| A1ZM017379_AT | NC0199638 | 2 | 79.6 | −0.06614 | 0.91 |
| A1ZM017379_AT | NC0172302 | 2 | 94.0 | −0.06477 | 0.90 |
| A1ZM017379_AT | NC0067577 | 4 | 59.3 | 0.13409 | 1.85 |
| A1ZM017379_AT | NC0171755 | 4 | 77.8 | −0.06392 | 0.88 |
| A1ZM017379_AT | NC0173414 | 4 | 78.9 | 0.07043 | 0.97 |
| A1ZM017379_AT | NC0199832 | 4 | 84.7 | −0.06119 | 0.85 |
| A1ZM017379_AT | NC0038478 | 5 | 96.0 | 0.06122 | 0.85 |
| A1ZM017379_AT | NC0173990 | 5 | 98.9 | −0.07374 | 1.02 |
| A1ZM017379_AT | NC0200212 | 5 | 176.4 | 0.07089 | 0.98 |
| A1ZM017379_AT | NC0173452 | 5 | 177.7 | 0.07364 | 1.02 |
| A1ZM017379_AT | NC0173900 | 6 | 38.4 | −0.06144 | 0.85 |
| A1ZM017379_AT | NC0199614 | 7 | 108.4 | −0.06169 | 0.85 |
| A1ZM017379_AT | NC0199339 | 7 | 150.8 | 0.07783 | 1.08 |
| A1ZM017379_AT | NC0199896 | 8 | 96.4 | 0.06164 | 0.85 |
| A1ZM017379_AT | NC0014545 | 8 | 155.1 | −0.06086 | 0.84 |
| A1ZM017379_AT | NC0010740 | 9 | 0.0 | 0.07944 | 1.10 |
| A1ZM017379_AT | NC0056940 | 9 | 0.4 | 0.06944 | 0.96 |
| A1ZM019225_AT | NC0199417 | 1 | 54.3 | −0.05653 | 0.99 |
| A1ZM019225_AT | NC0199318 | 1 | 85.9 | −0.05985 | 1.05 |
| A1ZM019225_AT | NC0157255 | 1 | 87.7 | −0.04905 | 0.86 |
| A1ZM019225_AT | NC0157397 | 1 | 87.7 | −0.04747 | 0.83 |
| A1ZM019225_AT | NC0029053 | 1 | 88.1 | 0.05760 | 1.01 |
| A1ZM019225_AT | NC0199760 | 1 | 179.1 | −0.05201 | 0.91 |
| A1ZM019225_AT | NC0196949 | 2 | 43.4 | −0.04801 | 0.84 |
| A1ZM019225_AT | NC0013275 | 2 | 49.7 | −0.06374 | 1.12 |
| A1ZM019225_AT | NC0170324 | 2 | 94.0 | −0.05609 | 0.98 |
| A1ZM019225_AT | NC0172302 | 2 | 94.0 | −0.05554 | 0.97 |
| A1ZM019225_AT | NC0112924 | 3 | 145.3 | −0.07012 | 1.23 |
| A1ZM019225_AT | NC0199794 | 3 | 146.0 | −0.05351 | 0.94 |
| A1ZM019225_AT | NC0029487 | 4 | 171.1 | −0.05109 | 0.89 |
| A1ZM019225_AT | NC0012281 | 5 | 106.5 | 0.04832 | 0.85 |
| A1ZM019225_AT | NC0200185 | 7 | 147.7 | −0.05169 | 0.91 |
| A1ZM019225_AT | NC0199331 | 7 | 173.1 | 0.05084 | 0.89 |
| A1ZM019225_AT | NC0199443 | 8 | 72.9 | −0.06256 | 1.10 |
| A1ZM019225_AT | NC0194645 | 8 | 76.5 | −0.05961 | 1.04 |
| A1ZM019225_AT | NC0199730 | 8 | 79.6 | −0.07042 | 1.23 |
| A1ZM019225_AT | NC0199735 | 8 | 84.4 | −0.06934 | 1.21 |
| A1ZM019225_AT | NC0199770 | 8 | 96.2 | −0.05152 | 0.90 |
| A1ZM019225_AT | NC0199896 | 8 | 96.4 | 0.07269 | 1.27 |
| A1ZM019225_AT | NC0173626 | 8 | 96.8 | −0.05668 | 0.99 |
| A1ZM019225_AT | NC0111472 | 8 | 103.3 | 0.04762 | 0.83 |
| A1ZM019225_AT | NC0199956 | 8 | 169.9 | 0.05334 | 0.93 |
| A1ZM020483_A_AT | NC0173400 | 1 | 32.2 | 0.06641 | 1.04 |
| A1ZM020483_A_AT | NC0199391 | 1 | 107.5 | 0.06107 | 0.95 |
| A1ZM020483_A_AT | NC0173951 | 1 | 229.4 | 0.08517 | 1.33 |
| A1ZM020483_A_AT | NC0200048 | 1 | 230.2 | −0.07692 | 1.20 |
| A1ZM020483_A_AT | NC0199645 | 3 | 12.1 | −0.06293 | 0.98 |
| A1ZM020483_A_AT | NC0190645 | 3 | 202.4 | 0.05844 | 0.91 |
| A1ZM020483_A_AT | NC0200099 | 5 | 171.1 | −0.05585 | 0.87 |
| A1ZM020483_A_AT | NC0033924 | 6 | 36.2 | −0.05541 | 0.86 |
| A1ZM020483_A_AT | NC0199354 | 7 | 87.4 | 0.05559 | 0.87 |
| A1ZM020483_A_AT | NC0173859 | 7 | 96.8 | −0.08069 | 1.26 |
| A1ZM020483_A_AT | NC0015314 | 7 | 99.0 | 0.05563 | 0.87 |
| A1ZM020483_A_AT | NC0071001 | 7 | 99.4 | −0.06199 | 0.97 |
| A1ZM020483_A_AT | NC0199889 | 7 | 99.4 | −0.06058 | 0.95 |
| A1ZM020483_A_AT | NC0079306 | 7 | 111.7 | 0.05855 | 0.91 |
| A1ZM020483_A_AT | NC0199879 | 7 | 112.1 | 0.06074 | 0.95 |
| A1ZM020483_A_AT | NC0199800 | 7 | 114.5 | 0.05811 | 0.91 |
| A1ZM020483_A_AT | NC0173358 | 7 | 131.0 | −0.05627 | 0.88 |
| A1ZM020483_A_AT | NC0200185 | 7 | 147.7 | −0.07213 | 1.13 |
| A1ZM020483_A_AT | NC0000129 | 8 | 16.5 | −0.06197 | 0.97 |
| A1ZM020483_A_AT | NC0199387 | 9 | 33.0 | −0.06590 | 1.03 |
| A1ZM021664_AT | NC0173332 | 1 | 33.3 | 0.03849 | 0.95 |
| A1ZM021664_AT | NC0199555 | 1 | 34.4 | −0.03435 | 0.85 |
| A1ZM021664_AT | NC0200044 | 1 | 47.5 | 0.04706 | 1.16 |
| A1ZM021664_AT | NC0143873 | 1 | 52.6 | 0.03757 | 0.92 |
| A1ZM021664_AT | NC0199417 | 1 | 54.3 | −0.03525 | 0.87 |
| A1ZM021664_AT | NC0010830 | 1 | 65.8 | 0.04824 | 1.19 |
| A1ZM021664_AT | NC0199971 | 1 | 66.1 | 0.04693 | 1.15 |
| A1ZM021664_AT | NC0158866 | 1 | 66.4 | −0.03601 | 0.89 |
| A1ZM021664_AT | NC0200213 | 1 | 67.4 | 0.05539 | 1.36 |
| A1ZM021664_AT | NC0199911 | 1 | 70.2 | 0.03720 | 0.92 |
| A1ZM021664_AT | NC0043266 | 1 | 71.3 | −0.03898 | 0.96 |
| A1ZM021664_AT | NC0173951 | 1 | 229.4 | 0.03363 | 0.83 |
| A1ZM021664_AT | NC0200138 | 1 | 238.1 | −0.03686 | 0.91 |
| A1ZM021664_AT | NC0199346 | 2 | 50.8 | −0.03658 | 0.90 |
| A1ZM021664_AT | NC0004901 | 2 | 131.6 | 0.03387 | 0.83 |
| A1ZM021664_AT | NC0188475 | 2 | 131.6 | 0.03748 | 0.92 |
| A1ZM021664_AT | NC0193437 | 2 | 131.6 | 0.03748 | 0.92 |
| A1ZM021664_AT | NC0003536 | 3 | 30.9 | −0.04131 | 1.02 |
| A1ZM021664_AT | NC0199913 | 3 | 152.8 | −0.03855 | 0.95 |
| A1ZM021664_AT | NC0174066 | 3 | 168.4 | 0.03575 | 0.88 |
| A1ZM021664_AT | NC0199837 | 4 | 46.8 | −0.03589 | 0.88 |
| A1ZM021664_AT | NC0174148 | 5 | 1.8 | 0.03515 | 0.86 |
| A1ZM021664_AT | NC0036529 | 6 | 43.2 | 0.05323 | 1.31 |
| A1ZM021664_AT | NC0158130 | 6 | 66.3 | −0.03587 | 0.88 |
| A1ZM021664_AT | NC0173859 | 7 | 96.8 | −0.04351 | 1.07 |
| A1ZM021664_AT | NC0199614 | 7 | 108.4 | −0.05376 | 1.32 |
| A1ZM021664_AT | NC0199800 | 7 | 114.5 | 0.03899 | 0.96 |
| A1ZM021664_AT | NC0199355 | 7 | 138.4 | −0.03508 | 0.86 |
| A1ZM021664_AT | NC0200185 | 7 | 147.7 | −0.05021 | 1.24 |
| A1ZM021664_AT | NC0173751 | 7 | 152.4 | 0.04045 | 1.00 |
| A1ZM021664_AT | NC0187978 | 7 | 165.7 | 0.03661 | 0.90 |
| A1ZM021664_AT | NC0199331 | 7 | 173.1 | 0.04413 | 1.09 |
| A1ZM021664_AT | NC0173490 | 8 | 59.0 | 0.03519 | 0.87 |
| A1ZM021664_AT | NC0199563 | 8 | 115.6 | 0.04448 | 1.09 |
| A1ZM021664_AT | NC0173574 | 9 | 36.6 | −0.04092 | 1.01 |
| A1ZM021664_AT | NC0173530 | 9 | 78.1 | 0.03837 | 0.94 |
| A1ZM021664_AT | NC0014240 | 9 | 103.5 | −0.04748 | 1.17 |
| A1ZM021664_AT | NC0200144 | 9 | 110.4 | 0.04075 | 1.00 |
| A1ZM021664_AT | NC0173478 | 9 | 122.4 | 0.03760 | 0.92 |
| A1ZM021664_AT | NC0173382 | 10 | 27.7 | 0.04053 | 1.00 |
| A1ZM021664_AT | NC0199635 | 10 | 106.4 | 0.03455 | 0.85 |
| A1ZM023477_S_AT | NC0199556 | 1 | 208.0 | −0.09500 | 0.90 |
| A1ZM023477_S_AT | NC0013275 | 2 | 49.7 | −0.09499 | 0.90 |
| A1ZM023477_S_AT | NC0199346 | 2 | 50.8 | −0.08919 | 0.85 |
| A1ZM023477_S_AT | NC0173731 | 2 | 138.4 | −0.08730 | 0.83 |
| A1ZM023477_S_AT | NC0003536 | 3 | 30.9 | −0.09965 | 0.95 |
| A1ZM023477_S_AT | NC0011194 | 5 | 29.3 | 0.09735 | 0.93 |
| A1ZM023477_S_AT | NC0188018 | 5 | 85.4 | 0.08641 | 0.82 |
| A1ZM023477_S_AT | NC0042917 | 6 | 36.4 | −0.08706 | 0.83 |
| A1ZM023477_S_AT | NC0036529 | 6 | 43.2 | 0.14186 | 1.35 |
| A1ZM023477_S_AT | NC0028658 | 6 | 52.7 | 0.17865 | 1.70 |
| A1ZM023477_S_AT | NC0143950 | 6 | 66.0 | 0.09256 | 0.88 |
| A1ZM023477_S_AT | NC0199535 | 6 | 75.4 | 0.09524 | 0.91 |
| A1ZM023477_S_AT | NC0199480 | 6 | 99.9 | 0.09387 | 0.89 |
| A1ZM023477_S_AT | NC0200165 | 6 | 99.9 | 0.11001 | 1.05 |
| A1ZM023477_S_AT | NC0173876 | 6 | 104.2 | 0.09092 | 0.87 |
| A1ZM023477_S_AT | NC0195223 | 6 | 118.6 | −0.09705 | 0.92 |
| A1ZM024567_AT | NC0173858 | 1 | 43.8 | −0.08484 | 0.99 |
| A1ZM024567_AT | NC0199467 | 1 | 96.7 | 0.07960 | 0.93 |
| A1ZM024567_AT | NC0199827 | 1 | 103.4 | −0.08187 | 0.95 |
| A1ZM024567_AT | NC0188455 | 1 | 113.3 | −0.08619 | 1.00 |
| A1ZM024567_AT | NC0199461 | 1 | 176.7 | 0.09361 | 1.09 |
| A1ZM024567_AT | NC0013275 | 2 | 49.7 | 0.08840 | 1.03 |
| A1ZM024567_AT | NC0011181 | 2 | 57.2 | −0.08662 | 1.01 |
| A1ZM024567_AT | NC0199522 | 3 | 90.3 | 0.08311 | 0.97 |
| A1ZM024567_AT | NC0173414 | 4 | 78.9 | −0.07909 | 0.92 |
| A1ZM024567_AT | NC0029487 | 4 | 171.1 | 0.08968 | 1.04 |
| A1ZM024567_AT | NC0199826 | 4 | 190.8 | 0.08471 | 0.99 |
| A1ZM024567_AT | NC0199425 | 5 | 81.2 | 0.08260 | 0.96 |
| A1ZM024567_AT | NC0188018 | 5 | 85.4 | −0.08564 | 1.00 |
| A1ZM024567_AT | NC0199735 | 8 | 84.4 | 0.08332 | 0.97 |
| A1ZM024567_AT | NC0145457 | 8 | 91.1 | −0.08576 | 1.00 |
| A1ZM024567_AT | NC0174094 | 8 | 91.1 | 0.09647 | 1.12 |
| A1ZM025661_AT | NC0200044 | 1 | 47.5 | 0.09124 | 1.01 |
| A1ZM025661_AT | NC0170324 | 2 | 94.0 | −0.07836 | 0.87 |
| A1ZM025661_AT | NC0172302 | 2 | 94.0 | −0.07992 | 0.88 |
| A1ZM025661_AT | NC0109315 | 3 | 77.4 | −0.07482 | 0.83 |
| A1ZM025661_AT | NC0199803 | 3 | 85.6 | −0.08863 | 0.98 |
| A1ZM025661_AT | NC0173436 | 3 | 86.0 | −0.13897 | 1.53 |
| A1ZM025661_AT | NC0199763 | 3 | 86.0 | 0.08728 | 0.96 |
| A1ZM025661_AT | NC0199947 | 3 | 86.4 | 0.10339 | 1.14 |
| A1ZM025661_AT | NC0199372 | 3 | 87.1 | 0.08128 | 0.90 |
| A1ZM025661_AT | NC0199589 | 3 | 91.5 | 0.10045 | 1.11 |
| A1ZM025661_AT | NC0199676 | 3 | 91.5 | 0.11469 | 1.27 |
| A1ZM025661_AT | NC0077263 | 4 | 50.6 | −0.07301 | 0.81 |
| A1ZM025661_AT | NC0200212 | 5 | 176.4 | 0.07555 | 0.83 |
| A1ZM025661_AT | NC0199915 | 6 | 84.9 | −0.08163 | 0.90 |
| A1ZM025661_AT | NC0187978 | 7 | 165.7 | 0.07929 | 0.88 |
| A1ZM025661_AT | NC0199730 | 8 | 79.6 | −0.07372 | 0.81 |
| A1ZM025769_AT | NC0013275 | 2 | 49.7 | 0.06653 | 1.01 |
| A1ZM025769_AT | NC0199346 | 2 | 50.8 | 0.09069 | 1.37 |
| A1ZM025769_AT | NC0002630 | 2 | 54.1 | −0.06485 | 0.98 |
| A1ZM025769_AT | NC0199524 | 2 | 141.5 | −0.06229 | 0.94 |
| A1ZM025769_AT | NC0030963 | 3 | 87.0 | −0.06183 | 0.94 |
| A1ZM025769_AT | NC0200081 | 3 | 115.9 | 0.06965 | 1.06 |
| A1ZM025769_AT | NC0199794 | 3 | 146.0 | 0.06877 | 1.04 |
| A1ZM025769_AT | NC0200120 | 5 | 75.4 | −0.08028 | 1.22 |
| A1ZM025769_AT | NC0199447 | 5 | 80.6 | 0.06054 | 0.92 |
| A1ZM025769_AT | NC0188018 | 5 | 85.4 | −0.05742 | 0.87 |
| A1ZM025769_AT | NC0078090 | 5 | 141.8 | 0.05658 | 0.86 |
| A1ZM025769_AT | NC0173396 | 5 | 143.1 | 0.07140 | 1.08 |
| A1ZM025769_AT | NC0042059 | 5 | 143.2 | 0.07263 | 1.10 |
| A1ZM025769_AT | NC0173843 | 5 | 143.2 | −0.05970 | 0.90 |
| A1ZM025769_AT | NC0033924 | 6 | 36.2 | 0.06348 | 0.96 |
| A1ZM025769_AT | NC0042917 | 6 | 36.4 | 0.05622 | 0.85 |
| A1ZM025769_AT | NC0195223 | 6 | 118.6 | 0.05601 | 0.85 |
| A1ZM025769_AT | NC0173794 | 6 | 131.1 | 0.06649 | 1.01 |
| A1ZM025769_AT | NC0199685 | 7 | 28.6 | 0.06401 | 0.97 |
| A1ZM025769_AT | NC0199331 | 7 | 173.1 | −0.05964 | 0.90 |
| A1ZM025982_AT | NC0194645 | 8 | 76.5 | 0.13598 | 0.94 |
| A1ZM025982_AT | NC0199730 | 8 | 79.6 | 0.17787 | 1.23 |
| A1ZM025982_AT | NC0199735 | 8 | 84.4 | 0.12829 | 0.88 |
| A1ZM025982_AT | NC0199770 | 8 | 96.2 | 0.14535 | 1.00 |
| A1ZM025982_AT | NC0199896 | 8 | 96.4 | −0.14701 | 1.01 |
| A1ZM025982_AT | NC0173626 | 8 | 96.8 | 0.12956 | 0.89 |
| A1ZM026053_AT | NC0036726 | 1 | 35.2 | 0.03990 | 0.80 |
| A1ZM026053_AT | NC0196949 | 2 | 43.4 | 0.05657 | 1.14 |
| A1ZM026053_AT | NC0013275 | 2 | 49.7 | 0.04975 | 1.00 |
| A1ZM026053_AT | NC0199741 | 3 | 125.7 | 0.04102 | 0.83 |
| A1ZM026053_AT | NC0015965 | 3 | 140.8 | 0.05190 | 1.05 |
| A1ZM026053_AT | NC0199921 | 3 | 141.1 | −0.04084 | 0.82 |
| A1ZM026053_AT | NC0199794 | 3 | 146.0 | 0.04055 | 0.82 |
| A1ZM026053_AT | NC0190032 | 3 | 147.8 | 0.05220 | 1.05 |
| A1ZM026053_AT | NC0175374 | 4 | 21.6 | 0.04212 | 0.85 |
| A1ZM026053_AT | NC0199978 | 4 | 70.6 | 0.03976 | 0.80 |
| A1ZM026053_AT | NC0199376 | 5 | 54.4 | −0.04168 | 0.84 |
| A1ZM026053_AT | NC0200112 | 6 | 105.6 | −0.04211 | 0.85 |
| A1ZM026053_AT | NC0199723 | 7 | 40.4 | −0.05134 | 1.03 |
| A1ZM026053_AT | NC0173476 | 7 | 44.2 | 0.04420 | 0.89 |
| A1ZM026053_AT | NC0158373 | 8 | 148.9 | −0.04189 | 0.84 |
| A1ZM026053_AT | NC0199880 | 8 | 161.8 | 0.04418 | 0.89 |
| A1ZM026053_AT | NC0029744 | 9 | 67.4 | −0.06562 | 1.32 |
| A1ZM026053_AT | NC0173953 | 9 | 67.4 | 0.05924 | 1.19 |
| A1ZM026053_AT | NC0020872 | 9 | 76.3 | 0.05112 | 1.03 |
| A1ZM026053_AT | NC0173530 | 9 | 78.1 | −0.05188 | 1.05 |
| A1ZM026053_AT | NC0199722 | 9 | 79.6 | −0.06739 | 1.36 |
| A1ZM026053_AT | NC0153579 | 9 | 97.3 | 0.04037 | 0.81 |
| A1ZM026053_AT | NC0014240 | 9 | 103.5 | 0.04798 | 0.97 |
| A1ZM026053_AT | NC0030512 | 9 | 103.6 | 0.06668 | 1.34 |
| A1ZM026053_AT | NC0200144 | 9 | 110.4 | −0.06515 | 1.31 |
| A1ZM026053_AT | NC0051922 | 9 | 110.5 | −0.04482 | 0.90 |
| A1ZM026053_AT | NC0029024 | 9 | 116.9 | −0.04286 | 0.86 |
| A1ZM026053_AT | NC0199898 | 9 | 119.2 | −0.04667 | 0.94 |
| A1ZM026053_AT | NC0173478 | 9 | 122.4 | −0.05517 | 1.11 |
| A1ZM026053_AT | NC0174164 | 9 | 147.9 | 0.04397 | 0.89 |
| A1ZM026053_AT | NC0011002 | 10 | 66.5 | 0.04723 | 0.95 |
| A1ZM026886_AT | NC0200147 | 5 | 139.5 | −0.15008 | 0.98 |
| A1ZM026886_AT | NC0038733 | 7 | 106.5 | 0.15955 | 1.04 |
| A1ZM026886_AT | NC0200123 | 7 | 107.2 | 0.15847 | 1.03 |
| A1ZM026886_AT | NC0173530 | 9 | 78.1 | 0.14797 | 0.96 |
| A1ZM026886_AT | NC0199722 | 9 | 79.6 | 0.15101 | 0.98 |
| A1ZM030937_AT | NC0032745 | 1 | 94.5 | −0.04906 | 0.90 |
| A1ZM030937_AT | NC0173334 | 1 | 102.8 | 0.04836 | 0.89 |
| A1ZM030937_AT | NC0173484 | 1 | 103.2 | 0.05206 | 0.95 |
| A1ZM030937_AT | NC0199346 | 2 | 50.8 | −0.04534 | 0.83 |
| A1ZM030937_AT | NC0195038 | 3 | 74.2 | −0.05017 | 0.92 |
| A1ZM030937_AT | NC0109315 | 3 | 77.4 | −0.06267 | 1.15 |
| A1ZM030937_AT | NC0173641 | 3 | 80.9 | 0.06448 | 1.18 |
| A1ZM030937_AT | NC0199803 | 3 | 85.6 | −0.04631 | 0.85 |
| A1ZM030937_AT | NC0173436 | 3 | 86.0 | −0.08150 | 1.49 |
| A1ZM030937_AT | NC0173845 | 3 | 87.0 | −0.04699 | 0.86 |
| A1ZM030937_AT | NC0199589 | 3 | 91.5 | 0.07812 | 1.43 |
| A1ZM030937_AT | NC0199676 | 3 | 91.5 | 0.06862 | 1.26 |
| A1ZM030937_AT | NC0200156 | 3 | 96.1 | 0.04822 | 0.88 |
| A1ZM030937_AT | NC0015965 | 3 | 140.8 | −0.05089 | 0.93 |
| A1ZM030937_AT | NC0199794 | 3 | 146.0 | −0.05571 | 1.02 |
| A1ZM030937_AT | NC0199814 | 3 | 208.5 | −0.04626 | 0.85 |
| A1ZM030937_AT | NC0200132 | 4 | 128.7 | −0.04826 | 0.88 |
| A1ZM030937_AT | NC0192676 | 5 | 88.2 | 0.04633 | 0.85 |
| A1ZM030937_AT | NC0199805 | 8 | 51.7 | −0.04464 | 0.82 |
| A1ZM030937_AT | NC0199387 | 9 | 33.0 | −0.04520 | 0.83 |
| A1ZM030937_AT | NC0029744 | 9 | 67.4 | 0.04860 | 0.89 |
| A1ZM030937_AT | NC0173953 | 9 | 67.4 | −0.04649 | 0.85 |
| A1ZM030937_AT | NC0020872 | 9 | 76.3 | −0.05111 | 0.94 |
| A1ZM030937_AT | NC0173530 | 9 | 78.1 | 0.05879 | 1.08 |
| A1ZM030937_AT | NC0199722 | 9 | 79.6 | 0.05439 | 1.00 |
| A1ZM030937_AT | NC0014240 | 9 | 103.5 | −0.06198 | 1.13 |
| A1ZM030937_AT | NC0030512 | 9 | 103.6 | −0.05445 | 1.00 |
| A1ZM030937_AT | NC0200144 | 9 | 110.4 | 0.06019 | 1.10 |
| A1ZM030937_AT | NC0157951 | 10 | 48.3 | 0.04383 | 0.80 |
| A1ZM031333_AT | NC0013275 | 2 | 49.7 | −0.08936 | 0.94 |
| A1ZM031333_AT | NC0199346 | 2 | 50.8 | −0.08901 | 0.94 |
| A1ZM031333_AT | NC0009470 | 3 | 88.0 | 0.08570 | 0.90 |
| A1ZM031333_AT | NC0015965 | 3 | 140.8 | −0.09789 | 1.03 |
| A1ZM031333_AT | NC0199794 | 3 | 146.0 | −0.08035 | 0.85 |
| A1ZM031333_AT | NC0199913 | 3 | 152.8 | −0.07651 | 0.81 |
| A1ZM031333_AT | NC0173476 | 7 | 44.2 | −0.08394 | 0.89 |
| A1ZM031333_AT | NC0200185 | 7 | 147.7 | −0.07899 | 0.83 |
| A1ZM031333_AT | NC0020872 | 9 | 76.3 | −0.10514 | 1.11 |
| A1ZM031333_AT | NC0173530 | 9 | 78.1 | 0.11429 | 1.21 |
| A1ZM031333_AT | NC0199722 | 9 | 79.6 | 0.13037 | 1.38 |
| A1ZM031333_AT | NC0011002 | 10 | 66.5 | −0.09068 | 0.96 |
| A1ZM032249_AT | NC0173951 | 1 | 229.4 | 0.18168 | 0.92 |
| A1ZM032249_AT | NC0199346 | 2 | 50.8 | −0.07136 | 0.82 |
| A1ZM032249_AT | NC0199669 | 3 | 122.8 | 0.22196 | 1.09 |
| A1ZM032249_AT | NC0015965 | 3 | 140.8 | −0.06541 | 1.03 |
| A1ZM032249_AT | NC0199794 | 3 | 146.0 | −0.08412 | 0.96 |
| A1ZM032249_AT | NC0190032 | 3 | 147.8 | −0.04389 | 0.83 |
| A1ZM032249_AT | NC0173476 | 7 | 44.2 | −0.15596 | 0.84 |
| A1ZM032249_AT | NC0173435 | 7 | 71.6 | −0.28189 | 1.26 |
| A1ZM032249_AT | NC0199355 | 7 | 138.4 | −0.25414 | 1.21 |
| A1ZM032249_AT | NC0020872 | 9 | 76.3 | 0.01853 | 0.92 |
| A1ZM032249_AT | NC0173530 | 9 | 78.1 | 0.08986 | 1.05 |
| A1ZM032249_AT | NC0199722 | 9 | 79.6 | 0.03780 | 0.95 |
| A1ZM033184_AT | NC0009148 | 1 | 213.0 | 0.08631 | 1.00 |
| A1ZM033184_AT | NC0013275 | 2 | 49.7 | −0.08063 | 0.93 |
| A1ZM033184_AT | NC0199346 | 2 | 50.8 | −0.08481 | 0.98 |
| A1ZM033184_AT | NC0174062 | 2 | 129.5 | −0.07905 | 0.92 |
| A1ZM033184_AT | NC0003536 | 3 | 30.9 | −0.08241 | 0.96 |
| A1ZM033184_AT | NC0015965 | 3 | 140.8 | −0.09366 | 1.09 |
| A1ZM033184_AT | NC0035568 | 3 | 166.8 | 0.08196 | 0.95 |
| A1ZM033184_AT | NC0200093 | 5 | 131.8 | −0.09789 | 1.13 |
| A1ZM033184_AT | NC0200165 | 6 | 99.9 | 0.08093 | 0.94 |
| A1ZM033184_AT | NC0200185 | 7 | 147.7 | −0.09041 | 1.05 |
| A1ZM033184_AT | NC0199563 | 8 | 115.6 | 0.08328 | 0.97 |
| A1ZM033184_AT | NC0173530 | 9 | 78.1 | 0.08423 | 0.98 |
| A1ZM033184_AT | NC0199722 | 9 | 79.6 | 0.08384 | 0.97 |
| A1ZM033184_AT | NC0014240 | 9 | 103.5 | −0.08870 | 1.03 |
| A1ZM033184_AT | NC0011002 | 10 | 66.5 | −0.09359 | 1.08 |
| A1ZM033611_AT | NC0199555 | 1 | 34.4 | −0.07035 | 0.95 |
| A1ZM033611_AT | NC0043266 | 1 | 71.3 | −0.06642 | 0.90 |
| A1ZM033611_AT | NC0199346 | 2 | 50.8 | −0.07477 | 1.01 |
| A1ZM033611_AT | NC0002630 | 2 | 54.1 | 0.07037 | 0.95 |
| A1ZM033611_AT | NC0109315 | 3 | 77.4 | −0.07120 | 0.96 |
| A1ZM033611_AT | NC0112924 | 3 | 145.3 | −0.07228 | 0.98 |
| A1ZM033611_AT | NC0199388 | 7 | 62.8 | −0.06716 | 0.91 |
| A1ZM033611_AT | NC0200064 | 7 | 65.3 | 0.06625 | 0.90 |
| A1ZM033611_AT | NC0199834 | 8 | 84.0 | −0.07528 | 1.02 |
| A1ZM033611_AT | NC0199581 | 8 | 91.6 | 0.07413 | 1.00 |
| A1ZM033611_AT | NC0173350 | 8 | 104.0 | 0.07203 | 0.98 |
| A1ZM033611_AT | NC0199563 | 8 | 115.6 | 0.07883 | 1.07 |
| A1ZM033611_AT | NC0029744 | 9 | 67.4 | 0.07238 | 0.98 |
| A1ZM033611_AT | NC0173953 | 9 | 67.4 | −0.07353 | 1.00 |
| A1ZM033611_AT | NC0173530 | 9 | 78.1 | 0.07233 | 0.98 |
| A1ZM033611_AT | NC0199722 | 9 | 79.6 | 0.07202 | 0.98 |
| A1ZM033611_AT | NC0014240 | 9 | 103.5 | −0.09958 | 1.35 |
| A1ZM033611_AT | NC0030512 | 9 | 103.6 | −0.06705 | 0.91 |
| A1ZM033611_AT | NC0199810 | 10 | 48.3 | 0.07938 | 1.08 |
| A1ZM033956_AT | NC0199661 | 1 | 202.9 | −0.15110 | 0.99 |
| A1ZM033956_AT | NC0174062 | 2 | 129.5 | −0.16092 | 1.06 |
| A1ZM033956_AT | NC0174066 | 3 | 168.4 | 0.14772 | 0.97 |
| A1ZM033956_AT | NC0195223 | 6 | 118.6 | −0.15374 | 1.01 |
| A1ZM033956_AT | NC0199958 | 6 | 142.0 | −0.15727 | 1.03 |
| A1ZM033956_AT | NC0020611 | 7 | 10.0 | 0.15420 | 1.01 |
| A1ZM033956_AT | NC0174551 | 7 | 11.2 | −0.14694 | 0.97 |
| A1ZM033956_AT | NC0038733 | 7 | 106.5 | 0.14517 | 0.95 |
| A1ZM037009_AT | NC0188455 | 1 | 113.3 | 0.07076 | 0.92 |
| A1ZM037009_AT | NC0199760 | 1 | 179.1 | −0.07244 | 0.94 |
| A1ZM037009_AT | NC0199913 | 3 | 152.8 | −0.07420 | 0.96 |
| A1ZM037009_AT | NC0036529 | 6 | 43.2 | 0.06736 | 0.87 |
| A1ZM037009_AT | NC0199685 | 7 | 28.6 | −0.08234 | 1.07 |
| A1ZM037009_AT | NC0199937 | 7 | 31.2 | −0.08128 | 1.05 |
| A1ZM037009_AT | NC0173476 | 7 | 44.2 | −0.07114 | 0.92 |
| A1ZM037009_AT | NC0199735 | 8 | 84.4 | −0.08415 | 1.09 |
| A1ZM037009_AT | NC0199440 | 8 | 87.9 | 0.07660 | 0.99 |
| A1ZM037009_AT | NC0145457 | 8 | 91.1 | 0.08299 | 1.07 |
| A1ZM037009_AT | NC0174094 | 8 | 91.1 | −0.07024 | 0.91 |
| A1ZM037009_AT | NC0199563 | 8 | 115.6 | 0.07851 | 1.02 |
| A1ZM037009_AT | NC0020872 | 9 | 76.3 | −0.07795 | 1.01 |
| A1ZM037009_AT | NC0173530 | 9 | 78.1 | 0.08343 | 1.08 |
| A1ZM037009_AT | NC0199722 | 9 | 79.6 | 0.08266 | 1.07 |
| A1ZM039148_AT | NC0003536 | 3 | 30.9 | −0.13051 | 1.13 |
| A1ZM039148_AT | NC0015965 | 3 | 140.8 | −0.12034 | 1.04 |
| A1ZM039148_AT | NC0190032 | 3 | 147.8 | −0.12714 | 1.10 |
| A1ZM039148_AT | NC0199913 | 3 | 152.8 | −0.11006 | 0.96 |
| A1ZM039148_AT | NC0199485 | 3 | 175.1 | 0.10710 | 0.93 |
| A1ZM039148_AT | NC0200093 | 5 | 131.8 | −0.11526 | 1.00 |
| A1ZM039148_AT | NC0173584 | 7 | 8.5 | 0.10646 | 0.92 |
| A1ZM039148_AT | NC0174551 | 7 | 11.2 | −0.10701 | 0.93 |
| A1ZM039148_AT | NC0173476 | 7 | 44.2 | −0.10752 | 0.93 |
| A1ZM039148_AT | NC0199805 | 8 | 51.7 | −0.11554 | 1.00 |
| A1ZM039148_AT | NC0199563 | 8 | 115.6 | 0.11762 | 1.02 |
| A1ZM043521_AT | NC0173400 | 1 | 32.2 | 0.05198 | 0.88 |
| A1ZM043521_AT | NC0200044 | 1 | 47.5 | 0.05409 | 0.91 |
| A1ZM043521_AT | NC0143873 | 1 | 52.6 | 0.05787 | 0.98 |
| A1ZM043521_AT | NC0000399 | 3 | 121.1 | −0.05194 | 0.88 |
| A1ZM043521_AT | NC0199741 | 3 | 125.7 | −0.06620 | 1.12 |
| A1ZM043521_AT | NC0015965 | 3 | 140.8 | −0.05091 | 0.86 |
| A1ZM043521_AT | NC0112924 | 3 | 145.3 | −0.06724 | 1.13 |
| A1ZM043521_AT | NC0199913 | 3 | 152.8 | −0.05637 | 0.95 |
| A1ZM043521_AT | NC0199877 | 4 | 127.9 | −0.05026 | 0.85 |
| A1ZM043521_AT | NC0024368 | 4 | 162.6 | −0.05703 | 0.96 |
| A1ZM043521_AT | NC0194397 | 6 | 38.4 | −0.06619 | 1.12 |
| A1ZM043521_AT | NC0199422 | 6 | 41.1 | 0.07342 | 1.24 |
| A1ZM043521_AT | NC0158250 | 7 | 24.3 | 0.07278 | 1.23 |
| A1ZM043521_AT | NC0199388 | 7 | 62.8 | −0.05027 | 0.85 |
| A1ZM043521_AT | NC0173859 | 7 | 96.8 | −0.06135 | 1.03 |
| A1ZM043521_AT | NC0199355 | 7 | 138.4 | −0.06030 | 1.02 |
| A1ZM043521_AT | NC0200185 | 7 | 147.7 | −0.06104 | 1.03 |
| A1ZM043521_AT | NC0000129 | 8 | 16.5 | −0.06128 | 1.03 |
| A1ZM043521_AT | NC0158373 | 8 | 148.9 | 0.05163 | 0.87 |
| A1ZM043521_AT | NC0173530 | 9 | 78.1 | 0.04991 | 0.84 |
| A1ZM043521_AT | NC0199722 | 9 | 79.6 | 0.04988 | 0.84 |
| A1ZM043521_AT | NC0010789 | 9 | 96.9 | 0.06449 | 1.09 |
| A1ZM043521_AT | NC0014240 | 9 | 103.5 | −0.05926 | 1.00 |
| A1ZM043521_AT | NC0173382 | 10 | 27.7 | 0.06290 | 1.06 |
| A1ZM043521_AT | NC0199810 | 10 | 48.3 | 0.05341 | 0.90 |
| A1ZM043521_AT | NC0173834 | 10 | 49.1 | −0.05819 | 0.98 |
| A1ZM043521_AT | NC0197151 | 10 | 92.4 | 0.05031 | 0.85 |
| A1ZM043521_AT | NC0199635 | 10 | 106.4 | 0.07644 | 1.29 |
| A1ZM043652_S_AT | NC0199461 | 1 | 176.7 | 0.06976 | 0.95 |
| A1ZM043652_S_AT | NC0013275 | 2 | 49.7 | 0.06169 | 0.84 |
| A1ZM043652_S_AT | NC0199465 | 3 | 81.6 | −0.07271 | 1.00 |
| A1ZM043652_S_AT | NC0199853 | 3 | 81.6 | 0.06779 | 0.93 |
| A1ZM043652_S_AT | NC0199803 | 3 | 85.6 | 0.07606 | 1.04 |
| A1ZM043652_S_AT | NC0173436 | 3 | 86.0 | 0.06687 | 0.92 |
| A1ZM043652_S_AT | NC0198241 | 3 | 102.0 | 0.06169 | 0.84 |
| A1ZM043652_S_AT | NC0199826 | 4 | 190.8 | 0.06760 | 0.93 |
| A1ZM043652_S_AT | NC0199614 | 7 | 108.4 | 0.06164 | 0.84 |
| A1ZM043652_S_AT | NC0199730 | 8 | 79.6 | 0.06397 | 0.88 |
| A1ZM043652_S_AT | NC0199735 | 8 | 84.4 | 0.07629 | 1.04 |
| A1ZM043652_S_AT | NC0199770 | 8 | 96.2 | 0.08659 | 1.19 |
| A1ZM043652_S_AT | NC0199896 | 8 | 96.4 | −0.06912 | 0.95 |
| A1ZM043652_S_AT | NC0173626 | 8 | 96.8 | 0.09984 | 1.37 |
| A1ZM043652_S_AT | NC0111472 | 8 | 103.3 | −0.08930 | 1.22 |
| A1ZM043652_S_AT | NC0107256 | 9 | 68.5 | −0.06532 | 0.89 |
| A1ZM044683_X_AT | NC0010830 | 1 | 65.8 | −0.10186 | 1.09 |
| A1ZM044683_X_AT | NC0158866 | 1 | 66.4 | 0.07810 | 0.84 |
| A1ZM044683_X_AT | NC0199911 | 1 | 70.2 | −0.08326 | 0.89 |
| A1ZM044683_X_AT | NC0173453 | 1 | 172.2 | −0.08166 | 0.88 |
| A1ZM044683_X_AT | NC0015937 | 1 | 197.7 | −0.09149 | 0.98 |
| A1ZM044683_X_AT | NC0199310 | 2 | 75.7 | 0.07554 | 0.81 |
| A1ZM044683_X_AT | NC0174061 | 2 | 118.1 | 0.10556 | 1.13 |
| A1ZM044683_X_AT | NC0200164 | 2 | 120.4 | −0.11633 | 1.25 |
| A1ZM044683_X_AT | NC0174062 | 2 | 129.5 | 0.14585 | 1.56 |
| A1ZM044683_X_AT | NC0199713 | 2 | 139.5 | −0.09392 | 1.01 |
| A1ZM044683_X_AT | NC0187941 | 4 | 121.0 | −0.08747 | 0.94 |
| A1ZM044683_X_AT | NC0173919 | 4 | 142.6 | −0.07772 | 0.83 |
| A1ZM044683_X_AT | NC0199707 | 4 | 144.0 | −0.07868 | 0.84 |
| A1ZM044683_X_AT | NC0199421 | 4 | 156.3 | 0.07624 | 0.82 |
| A1ZM044683_X_AT | NC0199468 | 4 | 173.5 | −0.09507 | 1.02 |
| A1ZM044683_X_AT | NC0020611 | 7 | 10.0 | −0.09100 | 0.98 |
| A1ZM044683_X_AT | NC0200123 | 7 | 107.2 | 0.07672 | 0.82 |
| A1ZM045717_AT | NC0013275 | 2 | 49.7 | −0.09518 | 0.89 |
| A1ZM045717_AT | NC0173731 | 2 | 138.4 | −0.09861 | 0.93 |
| A1ZM045717_AT | NC0003536 | 3 | 30.9 | −0.11558 | 1.09 |
| A1ZM045717_AT | NC0199853 | 3 | 81.6 | 0.10200 | 0.96 |
| A1ZM045717_AT | NC0015965 | 3 | 140.8 | −0.11562 | 1.09 |
| A1ZM045717_AT | NC0174066 | 3 | 168.4 | 0.10456 | 0.98 |
| A1ZM045717_AT | NC0200165 | 6 | 99.9 | 0.10037 | 0.94 |
| A1ZM045717_AT | NC0195223 | 6 | 118.6 | −0.09872 | 0.93 |
| A1ZM045717_AT | NC0200185 | 7 | 147.7 | −0.14206 | 1.34 |
| A1ZM045717_AT | NC0173530 | 9 | 78.1 | 0.09117 | 0.86 |
| A1ZM045717_AT | NC0011002 | 10 | 66.5 | −0.09638 | 0.91 |
| A1ZM047966_X_AT | NC0173332 | 1 | 33.3 | 0.04523 | 0.87 |
| A1ZM047966_X_AT | NC0199555 | 1 | 34.4 | −0.04617 | 0.89 |
| A1ZM047966_X_AT | NC0200044 | 1 | 47.5 | 0.04632 | 0.89 |
| A1ZM047966_X_AT | NC0143873 | 1 | 52.6 | 0.04306 | 0.83 |
| A1ZM047966_X_AT | NC0157255 | 1 | 87.7 | −0.07274 | 1.40 |
| A1ZM047966_X_AT | NC0157397 | 1 | 87.7 | −0.06028 | 1.16 |
| A1ZM047966_X_AT | NC0029053 | 1 | 88.1 | 0.07229 | 1.39 |
| A1ZM047966_X_AT | NC0171154 | 1 | 88.1 | 0.06311 | 1.22 |
| A1ZM047966_X_AT | NC0173852 | 1 | 88.1 | −0.05603 | 1.08 |
| A1ZM047966_X_AT | NC0199954 | 2 | 54.1 | −0.05162 | 0.99 |
| A1ZM047966_X_AT | NC0170324 | 2 | 94.0 | −0.04862 | 0.94 |
| A1ZM047966_X_AT | NC0172302 | 2 | 94.0 | −0.05675 | 1.09 |
| A1ZM047966_X_AT | NC0188475 | 2 | 131.6 | 0.04226 | 0.81 |
| A1ZM047966_X_AT | NC0193437 | 2 | 131.6 | 0.04226 | 0.81 |
| A1ZM047966_X_AT | NC0200126 | 3 | 81.8 | −0.04968 | 0.96 |
| A1ZM047966_X_AT | NC0173436 | 3 | 86.0 | −0.04156 | 0.80 |
| A1ZM047966_X_AT | NC0199741 | 3 | 125.7 | −0.04974 | 0.96 |
| A1ZM047966_X_AT | NC0112924 | 3 | 145.3 | −0.05550 | 1.07 |
| A1ZM047966_X_AT | NC0173832 | 4 | 41.5 | −0.05933 | 1.14 |
| A1ZM047966_X_AT | NC0173929 | 5 | 11.7 | 0.04555 | 0.88 |
| A1ZM047966_X_AT | NC0020696 | 6 | 38.5 | 0.04196 | 0.81 |
| A1ZM047966_X_AT | NC0199915 | 6 | 84.9 | −0.04438 | 0.86 |
| A1ZM047966_X_AT | NC0200165 | 6 | 99.9 | 0.04416 | 0.85 |
| A1ZM047966_X_AT | NC0199614 | 7 | 108.4 | −0.04327 | 0.83 |
| A1ZM047966_X_AT | NC0187978 | 7 | 165.7 | 0.04987 | 0.96 |
| A1ZM047966_X_AT | NC0199331 | 7 | 173.1 | 0.06183 | 1.19 |
| A1ZM047966_X_AT | NC0173490 | 8 | 59.0 | 0.04407 | 0.85 |
| A1ZM047966_X_AT | NC0194645 | 8 | 76.5 | −0.04919 | 0.95 |
| A1ZM047966_X_AT | NC0199730 | 8 | 79.6 | −0.05563 | 1.07 |
| A1ZM047966_X_AT | NC0199735 | 8 | 84.4 | −0.04545 | 0.88 |
| A1ZM047966_X_AT | NC0199896 | 8 | 96.4 | 0.05904 | 1.14 |
| A1ZM047966_X_AT | NC0199563 | 8 | 115.6 | 0.05193 | 1.00 |
| A1ZM049194_AT | NC0188455 | 1 | 113.3 | −0.15498 | 1.03 |
| A1ZM049194_AT | NC0199986 | 1 | 113.3 | −0.13866 | 0.92 |
| A1ZM049194_AT | NC0033320 | 2 | 173.6 | 0.17864 | 1.18 |
| A1ZM049194_AT | NC0199736 | 3 | 187.4 | 0.13749 | 0.91 |
| A1ZM049194_AT | NC0199388 | 7 | 62.8 | 0.15072 | 1.00 |
| A1ZM049194_AT | NC0035106 | 8 | 151.4 | −0.13989 | 0.93 |
| A1ZM049194_AT | NC0200107 | 9 | 65.9 | 0.15087 | 1.00 |
| A1ZM049860_AT | NC0199555 | 1 | 34.4 | −0.04466 | 0.80 |
| A1ZM049860_AT | NC0036726 | 1 | 35.2 | −0.04682 | 0.84 |
| A1ZM049860_AT | NC0199461 | 1 | 176.7 | −0.05193 | 0.93 |
| A1ZM049860_AT | NC0196949 | 2 | 43.4 | −0.05155 | 0.92 |
| A1ZM049860_AT | NC0000399 | 3 | 121.1 | −0.04823 | 0.87 |
| A1ZM049860_AT | NC0112924 | 3 | 145.3 | −0.05231 | 0.94 |
| A1ZM049860_AT | NC0199485 | 3 | 175.1 | 0.04549 | 0.82 |
| A1ZM049860_AT | NC0189286 | 4 | 177.0 | −0.05817 | 1.04 |
| A1ZM049860_AT | NC0199826 | 4 | 190.8 | −0.05297 | 0.95 |
| A1ZM049860_AT | NC0199675 | 5 | 45.9 | −0.04764 | 0.85 |
| A1ZM049860_AT | NC0199293 | 5 | 62.9 | −0.06489 | 1.16 |
| A1ZM049860_AT | NC0199447 | 5 | 80.6 | −0.05135 | 0.92 |
| A1ZM049860_AT | NC0194693 | 5 | 89.9 | −0.05182 | 0.93 |
| A1ZM049860_AT | NC0200165 | 6 | 99.9 | 0.05040 | 0.90 |
| A1ZM049860_AT | NC0195223 | 6 | 118.6 | −0.06379 | 1.14 |
| A1ZM049860_AT | NC0199409 | 6 | 118.6 | 0.06106 | 1.10 |
| A1ZM049860_AT | NC0194645 | 8 | 76.5 | −0.04655 | 0.84 |
| A1ZM049860_AT | NC0199730 | 8 | 79.6 | −0.04895 | 0.88 |
| A1ZM049860_AT | NC0199563 | 8 | 115.6 | 0.06209 | 1.11 |
| A1ZM049860_AT | NC0035106 | 8 | 151.4 | 0.06365 | 1.14 |
| A1ZM049860_AT | NC0033526 | 8 | 153.1 | −0.08533 | 1.53 |
| A1ZM049860_AT | NC0014545 | 8 | 155.1 | −0.06725 | 1.21 |
| A1ZM049860_AT | NC0056940 | 9 | 0.4 | 0.04689 | 0.84 |
| A1ZM052424_S_AT | NC0173400 | 1 | 32.2 | 0.20604 | 1.23 |
| A1ZM052424_S_AT | NC0200048 | 1 | 230.2 | −0.15107 | 0.90 |
| A1ZM052424_S_AT | NC0199373 | 4 | 146.5 | −0.15848 | 0.95 |
| A1ZM052424_S_AT | NC0199771 | 4 | 147.3 | −0.16240 | 0.97 |
| A1ZM052424_S_AT | NC0200120 | 5 | 75.4 | 0.17627 | 1.05 |
| A1ZM052424_S_AT | NC0173900 | 6 | 38.4 | −0.15573 | 0.93 |
| A1ZM052424_S_AT | NC0010740 | 9 | 0.0 | 0.16726 | 1.00 |
| A1ZM052424_S_AT | NC0174063 | 9 | 60.8 | −0.15674 | 0.94 |
| A1ZM053031_S_AT | NC0200156 | 3 | 96.1 | −0.23808 | 1.08 |
| A1ZM053031_S_AT | NC0199694 | 4 | 142.6 | −0.21371 | 0.97 |
| A1ZM053031_S_AT | NC0199470 | 5 | 2.0 | −0.20944 | 0.95 |
| A1ZM055950_S_AT | NC0032745 | 1 | 94.5 | 0.12701 | 1.02 |
| A1ZM055950_S_AT | NC0173334 | 1 | 102.8 | −0.12943 | 1.04 |
| A1ZM055950_S_AT | NC0173484 | 1 | 103.2 | −0.13569 | 1.09 |
| A1ZM055950_S_AT | NC0199986 | 1 | 113.3 | −0.11593 | 0.93 |
| A1ZM055950_S_AT | NC0009470 | 3 | 88.0 | 0.11345 | 0.91 |
| A1ZM056031_AT | NC0173400 | 1 | 32.2 | −0.07378 | 0.82 |
| A1ZM056031_AT | NC0032745 | 1 | 94.5 | 0.07942 | 0.88 |
| A1ZM056031_AT | NC0173334 | 1 | 102.8 | −0.07499 | 0.83 |
| A1ZM056031_AT | NC0173484 | 1 | 103.2 | −0.07676 | 0.85 |
| A1ZM056031_AT | NC0199986 | 1 | 113.3 | −0.10327 | 1.14 |
| A1ZM056031_AT | NC0199491 | 1 | 183.9 | −0.09474 | 1.05 |
| A1ZM056031_AT | NC0200135 | 1 | 188.2 | −0.07811 | 0.86 |
| A1ZM056031_AT | NC0188030 | 1 | 207.9 | −0.08376 | 0.93 |
| A1ZM056031_AT | NC0173436 | 3 | 86.0 | 0.08082 | 0.89 |
| A1ZM056031_AT | NC0199522 | 3 | 90.3 | 0.07372 | 0.81 |
| A1ZM056031_AT | NC0199589 | 3 | 91.5 | −0.14178 | 1.57 |
| A1ZM056031_AT | NC0199676 | 3 | 91.5 | −0.09551 | 1.06 |
| A1ZM056031_AT | NC0200156 | 3 | 96.1 | −0.10127 | 1.12 |
| A1ZM056031_AT | NC0015965 | 3 | 140.8 | 0.09528 | 1.05 |
| A1ZM056031_AT | NC0199794 | 3 | 146.0 | 0.09071 | 1.00 |
| A1ZM056031_AT | NC0190032 | 3 | 147.8 | 0.09035 | 1.00 |
| A1ZM056031_AT | NC0188678 | 3 | 201.5 | 0.07961 | 0.88 |
| A1ZM056158_AT | NC0173858 | 1 | 43.8 | 0.04889 | 0.85 |
| A1ZM056158_AT | NC0199454 | 1 | 136.7 | 0.04976 | 0.86 |
| A1ZM056158_AT | NC0199461 | 1 | 176.7 | −0.05268 | 0.91 |
| A1ZM056158_AT | NC0013275 | 2 | 49.7 | −0.05459 | 0.94 |
| A1ZM056158_AT | NC0200126 | 3 | 81.8 | −0.04747 | 0.82 |
| A1ZM056158_AT | NC0112481 | 3 | 183.6 | 0.05083 | 0.88 |
| A1ZM056158_AT | NC0195163 | 3 | 216.7 | 0.05444 | 0.94 |
| A1ZM056158_AT | NC0200106 | 5 | 124.4 | −0.04846 | 0.84 |
| A1ZM056158_AT | NC0079526 | 5 | 125.0 | −0.04863 | 0.84 |
| A1ZM056158_AT | NC0107877 | 5 | 132.7 | 0.05397 | 0.93 |
| A1ZM056158_AT | NC0036529 | 6 | 43.2 | 0.06204 | 1.07 |
| A1ZM056158_AT | NC0199443 | 8 | 72.9 | −0.05580 | 0.97 |
| A1ZM056158_AT | NC0194645 | 8 | 76.5 | −0.05995 | 1.04 |
| A1ZM056158_AT | NC0199730 | 8 | 79.6 | −0.05925 | 1.03 |
| A1ZM056158_AT | NC0199735 | 8 | 84.4 | −0.07448 | 1.29 |
| A1ZM056158_AT | NC0200184 | 8 | 91.1 | −0.05010 | 0.87 |
| A1ZM056158_AT | NC0199860 | 8 | 93.9 | −0.06080 | 1.05 |
| A1ZM056158_AT | NC0199770 | 8 | 96.2 | −0.07017 | 1.21 |
| A1ZM056158_AT | NC0199896 | 8 | 96.4 | 0.06574 | 1.14 |
| A1ZM056158_AT | NC0173626 | 8 | 96.8 | −0.07939 | 1.37 |
| A1ZM056158_AT | NC0111472 | 8 | 103.3 | 0.05842 | 1.01 |
| A1ZM056158_AT | NC0148189 | 9 | 91.0 | 0.05168 | 0.89 |
| A1ZM057110_AT | NC0172493 | 1 | 86.9 | −0.10390 | 0.85 |
| A1ZM057110_AT | NC0173465 | 1 | 86.9 | −0.11246 | 0.92 |
| A1ZM057110_AT | NC0002630 | 2 | 54.1 | −0.10237 | 0.84 |
| A1ZM057110_AT | NC0172718 | 5 | 72.0 | −0.12304 | 1.01 |
| A1ZM057110_AT | NC0200092 | 5 | 73.8 | −0.11893 | 0.97 |
| A1ZM057110_AT | NC0200120 | 5 | 75.4 | −0.17210 | 1.41 |
| A1ZM057110_AT | NC0173525 | 6 | 70.2 | 0.10074 | 0.82 |
| A1ZM057110_AT | NC0173962 | 6 | 96.7 | 0.15723 | 1.29 |
| A1ZM057110_AT | NC0199480 | 6 | 99.9 | −0.11289 | 0.92 |
| A1ZM057110_AT | NC0196573 | 8 | 66.1 | 0.11626 | 0.95 |
| A1ZM057110_AT | NC0199735 | 8 | 84.4 | 0.10179 | 0.83 |
| A1ZM057238_AT | NC0144205 | 1 | 38.5 | −0.06526 | 1.11 |
| A1ZM057238_AT | NC0200044 | 1 | 47.5 | 0.05369 | 0.91 |
| A1ZM057238_AT | NC0199346 | 2 | 50.8 | −0.05372 | 0.91 |
| A1ZM057238_AT | NC0002630 | 2 | 54.1 | 0.05891 | 1.00 |
| A1ZM057238_AT | NC0199853 | 3 | 81.6 | 0.05222 | 0.89 |
| A1ZM057238_AT | NC0015965 | 3 | 140.8 | −0.05865 | 0.99 |
| A1ZM057238_AT | NC0112924 | 3 | 145.3 | −0.05626 | 0.95 |
| A1ZM057238_AT | NC0199794 | 3 | 146.0 | −0.05461 | 0.93 |
| A1ZM057238_AT | NC0174066 | 3 | 168.4 | 0.05564 | 0.94 |
| A1ZM057238_AT | NC0190159 | 3 | 168.4 | 0.05467 | 0.93 |
| A1ZM057238_AT | NC0173452 | 5 | 177.7 | 0.07271 | 1.23 |
| A1ZM057238_AT | NC0200165 | 6 | 99.9 | 0.05441 | 0.92 |
| A1ZM057238_AT | NC0199958 | 6 | 142.0 | −0.08368 | 1.42 |
| A1ZM057238_AT | NC0194906 | 6 | 144.8 | 0.06253 | 1.06 |
| A1ZM057238_AT | NC0199312 | 7 | 65.9 | 0.06325 | 1.07 |
| A1ZM057238_AT | NC0000129 | 8 | 16.5 | −0.05206 | 0.88 |
| A1ZM057238_AT | NC0049890 | 8 | 41.2 | 0.06193 | 1.05 |
| A1ZM057238_AT | NC0199834 | 8 | 84.0 | −0.05193 | 0.88 |
| A1ZM057238_AT | NC0199464 | 9 | 66.5 | 0.05383 | 0.91 |
| A1ZM057238_AT | NC0002876 | 9 | 66.6 | 0.05434 | 0.92 |
| A1ZM057238_AT | NC0107256 | 9 | 68.5 | −0.06930 | 1.18 |
| A1ZM057238_AT | NC0173799 | 9 | 68.5 | 0.06056 | 1.03 |
| A1ZM057238_AT | NC0020872 | 9 | 76.3 | −0.05208 | 0.88 |
| A1ZM057238_AT | NC0173530 | 9 | 78.1 | 0.05862 | 0.99 |
| A1ZM057238_AT | NC0199722 | 9 | 79.6 | 0.05288 | 0.90 |
| A1ZM057238_AT | NC0028613 | 9 | 94.6 | −0.05356 | 0.91 |
| A1ZM057468_AT | NC0175336 | 1 | 43.5 | 0.06387 | 0.98 |
| A1ZM057468_AT | NC0173858 | 1 | 43.8 | −0.06943 | 1.07 |
| A1ZM057468_AT | NC0200188 | 1 | 44.1 | 0.06204 | 0.96 |
| A1ZM057468_AT | NC0199760 | 1 | 179.1 | 0.05950 | 0.92 |
| A1ZM057468_AT | NC0173409 | 2 | 32.7 | −0.07140 | 1.10 |
| A1ZM057468_AT | NC0013275 | 2 | 49.7 | 0.06516 | 1.00 |
| A1ZM057468_AT | NC0027262 | 2 | 57.3 | −0.05904 | 0.91 |
| A1ZM057468_AT | NC0009470 | 3 | 88.0 | −0.05992 | 0.92 |
| A1ZM057468_AT | NC0015965 | 3 | 140.8 | 0.06627 | 1.02 |
| A1ZM057468_AT | NC0199794 | 3 | 146.0 | 0.06215 | 0.96 |
| A1ZM057468_AT | NC0190032 | 3 | 147.8 | 0.06144 | 0.95 |
| A1ZM057468_AT | NC0200083 | 4 | 84.9 | −0.05970 | 0.92 |
| A1ZM057468_AT | NC0199945 | 6 | 39.4 | 0.07298 | 1.12 |
| A1ZM057468_AT | NC0036529 | 6 | 43.2 | −0.07957 | 1.23 |
| A1ZM057468_AT | NC0020611 | 7 | 10.0 | −0.06997 | 1.08 |
| A1ZM057468_AT | NC0199685 | 7 | 28.6 | 0.06089 | 0.94 |
| A1ZM057468_AT | NC0199984 | 7 | 60.4 | −0.06360 | 0.98 |
| A1ZM057468_AT | NC0199889 | 7 | 99.4 | 0.06073 | 0.94 |
| A1ZM057468_AT | NC0173530 | 9 | 78.1 | −0.05959 | 0.92 |
| A1ZM057468_AT | NC0199722 | 9 | 79.6 | −0.06904 | 1.06 |
| A1ZM057468_AT | NC0200144 | 9 | 110.4 | −0.06207 | 0.96 |
| A1ZM057725_A_AT | NC0191470 | 1 | 57.9 | −0.03977 | 0.87 |
| A1ZM057725_A_AT | NC0200081 | 3 | 115.9 | 0.04069 | 0.89 |
| A1ZM057725_A_AT | NC0199921 | 3 | 141.1 | −0.05204 | 1.13 |
| A1ZM057725_A_AT | NC0173814 | 4 | 140.2 | 0.04133 | 0.90 |
| A1ZM057725_A_AT | NC0199746 | 5 | 85.4 | 0.03958 | 0.86 |
| A1ZM057725_A_AT | NC0200093 | 5 | 131.8 | 0.03940 | 0.86 |
| A1ZM057725_A_AT | NC0033526 | 8 | 153.1 | 0.04609 | 1.00 |
| A1ZM057725_A_AT | NC0014545 | 8 | 155.1 | 0.04546 | 0.99 |
| A1ZM057725_A_AT | NC0010740 | 9 | 0.0 | −0.03995 | 0.87 |
| A1ZM057725_A_AT | NC0056940 | 9 | 0.4 | −0.04706 | 1.03 |
| A1ZM057725_A_AT | NC0171018 | 10 | 42.7 | 0.07297 | 1.59 |
| A1ZM057725_A_AT | NC0016041 | 10 | 43.7 | −0.04466 | 0.97 |
| A1ZM057725_A_AT | NC0146010 | 10 | 43.7 | −0.04529 | 0.99 |
| A1ZM057725_A_AT | NC0173336 | 10 | 45.0 | −0.04825 | 1.05 |
| A1ZM057725_A_AT | NC0173415 | 10 | 45.0 | 0.03848 | 0.84 |
| A1ZM057725_A_AT | NC0190754 | 10 | 45.0 | 0.03848 | 0.84 |
| A1ZM057725_A_AT | NC0199333 | 10 | 45.0 | 0.04223 | 0.92 |
| A1ZM057725_A_AT | NC0199574 | 10 | 45.2 | −0.03957 | 0.86 |
| A1ZM057725_A_AT | NC0157951 | 10 | 48.3 | −0.06158 | 1.34 |
| A1ZM057725_A_AT | NC0199686 | 10 | 49.1 | 0.04157 | 0.91 |
| A1ZM057725_A_AT | NC0143408 | 10 | 49.2 | −0.04206 | 0.92 |
| A1ZM057725_A_AT | NC0199366 | 10 | 51.6 | 0.05162 | 1.12 |
| A1ZM057725_A_AT | NC0040011 | 10 | 54.2 | −0.04170 | 0.91 |
| A1ZM057856_AT | NC0200044 | 1 | 47.5 | 0.05103 | 0.99 |
| A1ZM057856_AT | NC0143873 | 1 | 52.6 | 0.04272 | 0.83 |
| A1ZM057856_AT | NC0200213 | 1 | 67.4 | 0.05713 | 1.11 |
| A1ZM057856_AT | NC0173436 | 3 | 86.0 | −0.05152 | 1.00 |
| A1ZM057856_AT | NC0199589 | 3 | 91.5 | 0.05309 | 1.03 |
| A1ZM057856_AT | NC0199676 | 3 | 91.5 | 0.04799 | 0.93 |
| A1ZM057856_AT | NC0199814 | 3 | 208.5 | −0.04476 | 0.87 |
| A1ZM057856_AT | NC0199657 | 4 | 0.5 | −0.04300 | 0.83 |
| A1ZM057856_AT | NC0199824 | 4 | 13.6 | 0.04271 | 0.83 |
| A1ZM057856_AT | NC0199781 | 4 | 82.7 | −0.05716 | 1.11 |
| A1ZM057856_AT | NC0189552 | 5 | 63.8 | −0.04908 | 0.95 |
| A1ZM057856_AT | NC0200171 | 5 | 73.2 | 0.04993 | 0.97 |
| A1ZM057856_AT | NC0078090 | 5 | 141.8 | −0.04414 | 0.86 |
| A1ZM057856_AT | NC0042059 | 5 | 143.2 | −0.04916 | 0.95 |
| A1ZM057856_AT | NC0173843 | 5 | 143.2 | 0.04613 | 0.89 |
| A1ZM057856_AT | NC0199422 | 6 | 41.1 | 0.05016 | 0.97 |
| A1ZM057856_AT | NC0200199 | 7 | 12.4 | −0.04304 | 0.83 |
| A1ZM057856_AT | NC0199723 | 7 | 40.4 | −0.04648 | 0.90 |
| A1ZM057856_AT | NC0199648 | 7 | 62.5 | 0.05128 | 0.99 |
| A1ZM057856_AT | NC0173493 | 7 | 62.9 | 0.04739 | 0.92 |
| A1ZM057856_AT | NC0200064 | 7 | 65.3 | 0.04348 | 0.84 |
| A1ZM057856_AT | NC0200207 | 7 | 84.1 | −0.05408 | 1.05 |
| A1ZM057856_AT | NC0008799 | 7 | 87.0 | 0.06288 | 1.22 |
| A1ZM057856_AT | NC0199354 | 7 | 87.4 | 0.06461 | 1.25 |
| A1ZM057856_AT | NC0173859 | 7 | 96.8 | −0.07996 | 1.55 |
| A1ZM057856_AT | NC0015314 | 7 | 99.0 | 0.04511 | 0.87 |
| A1ZM057856_AT | NC0071001 | 7 | 99.4 | −0.05597 | 1.09 |
| A1ZM057856_AT | NC0199889 | 7 | 99.4 | −0.05885 | 1.14 |
| A1ZM057856_AT | NC0199614 | 7 | 108.4 | −0.04539 | 0.88 |
| A1ZM057856_AT | NC0199339 | 7 | 150.8 | 0.05037 | 0.98 |
| A1ZM058469_AT | NC0199803 | 3 | 85.6 | −0.14536 | 0.85 |
| A1ZM058469_AT | NC0173436 | 3 | 86.0 | −0.21419 | 1.25 |
| A1ZM058469_AT | NC0199947 | 3 | 86.4 | 0.18693 | 1.09 |
| A1ZM058469_AT | NC0199372 | 3 | 87.1 | 0.14177 | 0.83 |
| A1ZM058469_AT | NC0199589 | 3 | 91.5 | 0.13787 | 0.80 |
| A1ZM058469_AT | NC0199676 | 3 | 91.5 | 0.18896 | 1.10 |
| A1ZM061580_AT | NC0173400 | 1 | 32.2 | −0.09447 | 0.88 |
| A1ZM061580_AT | NC0173332 | 1 | 33.3 | −0.10510 | 0.98 |
| A1ZM061580_AT | NC0199555 | 1 | 34.4 | 0.14385 | 1.34 |
| A1ZM061580_AT | NC0035426 | 1 | 34.5 | −0.09403 | 0.88 |
| A1ZM061580_AT | NC0036726 | 1 | 35.2 | 0.10530 | 0.98 |
| A1ZM061580_AT | NC0173919 | 4 | 142.6 | −0.10371 | 0.97 |
| A1ZM061580_AT | NC0199575 | 4 | 142.6 | 0.11151 | 1.04 |
| A1ZM061580_AT | NC0199739 | 5 | 62.9 | 0.10562 | 0.99 |
| A1ZM061580_AT | NC0079519 | 5 | 63.1 | −0.11113 | 1.04 |
| A1ZM061580_AT | NC0194397 | 6 | 38.4 | 0.09573 | 0.89 |
| A1ZM061580_AT | NC0199860 | 8 | 93.9 | 0.09696 | 0.91 |
| A1ZM061606_X_AT | NC0199461 | 1 | 176.7 | −0.09419 | 1.03 |
| A1ZM061606_X_AT | NC0013275 | 2 | 49.7 | −0.11516 | 1.26 |
| A1ZM061606_X_AT | NC0199346 | 2 | 50.8 | −0.08002 | 0.87 |
| A1ZM061606_X_AT | NC0015965 | 3 | 140.8 | −0.09671 | 1.05 |
| A1ZM061606_X_AT | NC0199794 | 3 | 146.0 | −0.08644 | 0.94 |
| A1ZM061606_X_AT | NC0173962 | 6 | 96.7 | −0.09175 | 1.00 |
| A1ZM061606_X_AT | NC0199735 | 8 | 84.4 | −0.07938 | 0.87 |
| A1ZM061606_X_AT | NC0174094 | 8 | 91.1 | −0.09285 | 1.01 |
| A1ZM061606_X_AT | NC0199722 | 9 | 79.6 | 0.08361 | 0.91 |
| A1ZM062662_AT | NC0199415 | 2 | 84.7 | 0.11662 | 1.00 |
| A1ZM062662_AT | NC0174061 | 2 | 118.1 | −0.11292 | 0.97 |
| A1ZM062662_AT | NC0174062 | 2 | 129.5 | −0.11671 | 1.00 |
| A1ZM062662_AT | NC0173731 | 2 | 138.4 | −0.11862 | 1.01 |
| A1ZM062662_AT | NC0173436 | 3 | 86.0 | −0.11666 | 1.00 |
| A1ZM062662_AT | NC0199741 | 3 | 125.7 | −0.11365 | 0.97 |
| A1ZM062662_AT | NC0195223 | 6 | 118.6 | −0.12874 | 1.10 |
| A1ZM062662_AT | NC0199409 | 6 | 118.6 | 0.11429 | 0.98 |
| A1ZM062662_AT | NC0173476 | 7 | 44.2 | −0.11488 | 0.98 |
| A1ZM062662_AT | NC0200185 | 7 | 147.7 | −0.12476 | 1.07 |
| A1ZM062662_AT | NC0199722 | 9 | 79.6 | 0.11213 | 0.96 |
| A1ZM062662_AT | NC0011002 | 10 | 66.5 | −0.11237 | 0.96 |
| A1ZM063309_AT | NC0173951 | 1 | 229.4 | 0.14379 | 1.15 |
| A1ZM063309_AT | NC0013275 | 2 | 49.7 | −0.11625 | 0.93 |
| A1ZM063309_AT | NC0199346 | 2 | 50.8 | −0.13208 | 1.05 |
| A1ZM063309_AT | NC0174066 | 3 | 168.4 | 0.10976 | 0.88 |
| A1ZM063309_AT | NC0199750 | 6 | 81.6 | −0.12313 | 0.98 |
| A1ZM063309_AT | NC0199354 | 7 | 87.4 | 0.11748 | 0.94 |
| A1ZM063309_AT | NC0200185 | 7 | 147.7 | −0.13465 | 1.07 |
| A1ZM063309_AT | NC0049890 | 8 | 41.2 | 0.13420 | 1.07 |
| A1ZM063309_AT | NC0023268 | 9 | 84.5 | 0.11409 | 0.91 |
| A1ZM063309_AT | NC0014240 | 9 | 103.5 | −0.13391 | 1.07 |
| A1ZM063309_AT | NC0030512 | 9 | 103.6 | −0.11451 | 0.91 |
| A1ZM067500_AT | NC0199491 | 1 | 183.9 | 0.07835 | 0.89 |
| A1ZM067500_AT | NC0196949 | 2 | 43.4 | −0.08829 | 1.01 |
| A1ZM067500_AT | NC0013275 | 2 | 49.7 | −0.12334 | 1.41 |
| A1ZM067500_AT | NC0199346 | 2 | 50.8 | −0.07524 | 0.86 |
| A1ZM067500_AT | NC0170324 | 2 | 94.0 | −0.07637 | 0.87 |
| A1ZM067500_AT | NC0172302 | 2 | 94.0 | −0.08050 | 0.92 |
| A1ZM067500_AT | NC0200140 | 2 | 99.3 | −0.07575 | 0.86 |
| A1ZM067500_AT | NC0195038 | 3 | 74.2 | −0.07967 | 0.91 |
| A1ZM067500_AT | NC0109315 | 3 | 77.4 | −0.07688 | 0.88 |
| A1ZM067500_AT | NC0173436 | 3 | 86.0 | −0.08576 | 0.98 |
| A1ZM067500_AT | NC0199794 | 3 | 146.0 | −0.09025 | 1.03 |
| A1ZM067500_AT | NC0193457 | 3 | 216.6 | 0.07952 | 0.91 |
| A1ZM067500_AT | NC0195223 | 6 | 118.6 | −0.11449 | 1.31 |
| A1ZM067500_AT | NC0199409 | 6 | 118.6 | 0.09597 | 1.10 |
| A1ZM067500_AT | NC0200064 | 7 | 65.3 | 0.09251 | 1.06 |
| A1ZM067500_AT | NC0200039 | 8 | 55.3 | −0.07690 | 0.88 |
| A1ZM067500_AT | NC0199722 | 9 | 79.6 | 0.08278 | 0.94 |
| A1ZM067740_X_AT | NC0199346 | 2 | 50.8 | −0.10998 | 0.97 |
| A1ZM067740_X_AT | NC0173350 | 8 | 104.0 | 0.09548 | 0.84 |
| A1ZM067740_X_AT | NC0020872 | 9 | 76.3 | −0.10087 | 0.89 |
| A1ZM067740_X_AT | NC0173530 | 9 | 78.1 | 0.11435 | 1.01 |
| A1ZM067740_X_AT | NC0199722 | 9 | 79.6 | 0.11915 | 1.05 |
| A1ZM067740_X_AT | NC0014240 | 9 | 103.5 | −0.13644 | 1.20 |
| A1ZM069545_AT | NC0199461 | 1 | 176.7 | 0.05472 | 1.35 |
| A1ZM069545_AT | NC0013275 | 2 | 49.7 | 0.04804 | 1.19 |
| A1ZM069545_AT | NC0170324 | 2 | 94.0 | 0.04267 | 1.06 |
| A1ZM069545_AT | NC0172302 | 2 | 94.0 | 0.04211 | 1.04 |
| A1ZM069545_AT | NC0199297 | 2 | 100.2 | −0.04390 | 1.09 |
| A1ZM069545_AT | NC0199568 | 2 | 114.7 | 0.03882 | 0.96 |
| A1ZM069545_AT | NC0035094 | 2 | 116.9 | 0.05036 | 1.25 |
| A1ZM069545_AT | NC0199511 | 2 | 121.3 | −0.03593 | 0.89 |
| A1ZM069545_AT | NC0038699 | 2 | 121.8 | 0.04728 | 1.17 |
| A1ZM069545_AT | NC0004901 | 2 | 131.6 | −0.03728 | 0.92 |
| A1ZM069545_AT | NC0188475 | 2 | 131.6 | −0.03337 | 0.83 |
| A1ZM069545_AT | NC0193437 | 2 | 131.6 | −0.03337 | 0.83 |
| A1ZM069545_AT | NC0199851 | 2 | 165.2 | −0.03446 | 0.85 |
| A1ZM069545_AT | NC0200126 | 3 | 81.8 | 0.03739 | 0.92 |
| A1ZM069545_AT | NC0173790 | 3 | 83.2 | −0.03758 | 0.93 |
| A1ZM069545_AT | NC0199763 | 3 | 86.0 | −0.03741 | 0.92 |
| A1ZM069545_AT | NC0199947 | 3 | 86.4 | −0.03731 | 0.92 |
| A1ZM069545_AT | NC0173739 | 3 | 116.9 | −0.03857 | 0.95 |
| A1ZM069545_AT | NC0000399 | 3 | 121.1 | 0.05170 | 1.28 |
| A1ZM069545_AT | NC0015965 | 3 | 140.8 | 0.03821 | 0.94 |
| A1ZM069545_AT | NC0112924 | 3 | 145.3 | 0.03788 | 0.94 |
| A1ZM069545_AT | NC0199794 | 3 | 146.0 | 0.03895 | 0.96 |
| A1ZM069545_AT | NC0112481 | 3 | 183.6 | −0.03723 | 0.92 |
| A1ZM069545_AT | NC0067577 | 4 | 59.3 | −0.03513 | 0.87 |
| A1ZM069545_AT | NC0173568 | 4 | 150.5 | −0.03699 | 0.91 |
| A1ZM069545_AT | NC0195197 | 5 | 66.6 | 0.03837 | 0.95 |
| A1ZM069545_AT | NC0200171 | 5 | 73.2 | 0.04079 | 1.01 |
| A1ZM069545_AT | NC0056874 | 5 | 73.5 | −0.03396 | 0.84 |
| A1ZM069545_AT | NC0079526 | 5 | 125.0 | 0.03726 | 0.92 |
| A1ZM069545_AT | NC0107877 | 5 | 132.7 | −0.03445 | 0.85 |
| A1ZM069545_AT | NC0199915 | 6 | 84.9 | 0.03629 | 0.90 |
| A1ZM069545_AT | NC0195223 | 6 | 118.6 | 0.03432 | 0.85 |
| A1ZM069545_AT | NC0199409 | 6 | 118.6 | −0.03767 | 0.93 |
| A1ZM069545_AT | NC0199443 | 8 | 72.9 | 0.04287 | 1.06 |
| A1ZM069545_AT | NC0194645 | 8 | 76.5 | 0.05069 | 1.25 |
| A1ZM069545_AT | NC0199730 | 8 | 79.6 | 0.04403 | 1.09 |
| A1ZM069545_AT | NC0111472 | 8 | 103.3 | −0.04539 | 1.12 |
| A1ZM069545_AT | NC0033526 | 8 | 153.1 | 0.04763 | 1.18 |
| A1ZM069545_AT | NC0014545 | 8 | 155.1 | 0.03408 | 0.84 |
| A1ZM069545_AT | NC0028613 | 9 | 94.6 | 0.04019 | 0.99 |
| A1ZM069545_AT | NC0053108 | 10 | 61.2 | 0.03832 | 0.95 |
| A1ZM072906_AT | NC0013275 | 2 | 49.7 | −0.12449 | 0.96 |
| A1ZM072906_AT | NC0003536 | 3 | 30.9 | −0.13511 | 1.04 |
| A1ZM072906_AT | NC0195223 | 6 | 118.6 | −0.13512 | 1.04 |
| A1ZM072906_AT | NC0199409 | 6 | 118.6 | 0.12655 | 0.98 |
| A1ZM072906_AT | NC0199958 | 6 | 142.0 | −0.13363 | 1.03 |
| A1ZM072906_AT | NC0199312 | 7 | 65.9 | 0.12633 | 0.97 |
| A1ZM072906_AT | NC0199563 | 8 | 115.6 | 0.13684 | 1.05 |
| A1ZM072906_AT | NC0011002 | 10 | 66.5 | −0.11878 | 0.92 |
| A1ZM077940_X_AT | NC0199738 | 2 | 28.8 | 0.05337 | 0.82 |
| A1ZM077940_X_AT | NC0196898 | 2 | 33.5 | 0.06258 | 0.96 |
| A1ZM077940_X_AT | NC0013275 | 2 | 49.7 | −0.05481 | 0.85 |
| A1ZM077940_X_AT | NC0199851 | 2 | 165.2 | 0.06089 | 0.94 |
| A1ZM077940_X_AT | NC0033320 | 2 | 173.6 | −0.05286 | 0.81 |
| A1ZM077940_X_AT | NC0110714 | 2 | 190.1 | 0.05948 | 0.92 |
| A1ZM077940_X_AT | NC0173436 | 3 | 86.0 | −0.05726 | 0.88 |
| A1ZM077940_X_AT | NC0188678 | 3 | 201.5 | −0.08312 | 1.28 |
| A1ZM077940_X_AT | NC0018073 | 3 | 201.6 | −0.07971 | 1.23 |
| A1ZM077940_X_AT | NC0000189 | 3 | 215.3 | 0.07925 | 1.22 |
| A1ZM077940_X_AT | NC0193457 | 3 | 216.6 | 0.08565 | 1.32 |
| A1ZM077940_X_AT | NC0170468 | 3 | 216.7 | −0.06831 | 1.05 |
| A1ZM077940_X_AT | NC0195163 | 3 | 216.7 | 0.06041 | 0.93 |
| A1ZM077940_X_AT | NC0038211 | 4 | 141.1 | −0.06188 | 0.95 |
| A1ZM077940_X_AT | NC0173628 | 4 | 144.0 | −0.06191 | 0.95 |
| A1ZM077940_X_AT | NC0173518 | 4 | 152.5 | 0.06674 | 1.03 |
| A1ZM077940_X_AT | NC0200099 | 5 | 171.1 | −0.05578 | 0.86 |
| A1ZM077940_X_AT | NC0029612 | 6 | 57.8 | −0.06533 | 1.01 |
| A1ZM077940_X_AT | NC0199409 | 6 | 118.6 | 0.05459 | 0.84 |
| A1ZM077940_X_AT | NC0200186 | 6 | 131.0 | 0.05498 | 0.85 |
| A1ZM077940_X_AT | NC0013445 | 6 | 131.2 | −0.05634 | 0.87 |
| A1ZM077940_X_AT | NC0200199 | 7 | 12.4 | −0.06039 | 0.93 |
| A1ZM077940_X_AT | NC0113218 | 8 | 75.2 | −0.08328 | 1.28 |
| A1ZM077940_X_AT | NC0199574 | 10 | 45.2 | 0.05868 | 0.90 |
| A1ZM079744_AT | NC0196949 | 2 | 43.4 | −0.06124 | 0.93 |
| A1ZM079744_AT | NC0013275 | 2 | 49.7 | −0.09514 | 1.45 |
| A1ZM079744_AT | NC0199346 | 2 | 50.8 | −0.05850 | 0.89 |
| A1ZM079744_AT | NC0003536 | 3 | 30.9 | −0.05766 | 0.88 |
| A1ZM079744_AT | NC0199741 | 3 | 125.7 | −0.05682 | 0.86 |
| A1ZM079744_AT | NC0015965 | 3 | 140.8 | −0.08197 | 1.25 |
| A1ZM079744_AT | NC0112924 | 3 | 145.3 | −0.07165 | 1.09 |
| A1ZM079744_AT | NC0199794 | 3 | 146.0 | −0.07446 | 1.13 |
| A1ZM079744_AT | NC0199913 | 3 | 152.8 | −0.05720 | 0.87 |
| A1ZM079744_AT | NC0199750 | 6 | 81.6 | −0.06504 | 0.99 |
| A1ZM079744_AT | NC0200165 | 6 | 99.9 | 0.05777 | 0.88 |
| A1ZM079744_AT | NC0195223 | 6 | 118.6 | −0.07257 | 1.10 |
| A1ZM079744_AT | NC0199409 | 6 | 118.6 | 0.06111 | 0.93 |
| A1ZM079744_AT | NC0199958 | 6 | 142.0 | −0.05598 | 0.85 |
| A1ZM079744_AT | NC0200185 | 7 | 147.7 | −0.07289 | 1.11 |
| A1ZM079744_AT | NC0199331 | 7 | 173.1 | 0.06173 | 0.94 |
| A1ZM079744_AT | NC0199896 | 8 | 96.4 | 0.05674 | 0.86 |
| A1ZM079744_AT | NC0199810 | 10 | 48.3 | 0.05540 | 0.84 |
| A1ZM079744_AT | NC0011002 | 10 | 66.5 | −0.06117 | 0.93 |
| A1ZM081073_AT | NC0199760 | 1 | 179.1 | −0.14299 | 0.92 |
| A1ZM081073_AT | NC0173731 | 2 | 138.4 | −0.13913 | 0.90 |
| A1ZM081073_AT | NC0109315 | 3 | 77.4 | −0.17740 | 1.14 |
| A1ZM081073_AT | NC0173641 | 3 | 80.9 | 0.16817 | 1.09 |
| A1ZM081073_AT | NC0200132 | 4 | 128.7 | −0.14998 | 0.97 |
| A1ZM081073_AT | NC0199754 | 7 | 77.6 | 0.16367 | 1.06 |
| A1ZM081073_AT | NC0199722 | 9 | 79.6 | 0.13871 | 0.90 |
| A1ZM084532_AT | NC0196949 | 2 | 43.4 | −0.09024 | 0.99 |
| A1ZM084532_AT | NC0173731 | 2 | 138.4 | −0.09084 | 0.99 |
| A1ZM084532_AT | NC0173436 | 3 | 86.0 | −0.08827 | 0.97 |
| A1ZM084532_AT | NC0015965 | 3 | 140.8 | −0.08769 | 0.96 |
| A1ZM084532_AT | NC0112924 | 3 | 145.3 | −0.08738 | 0.96 |
| A1ZM084532_AT | NC0199794 | 3 | 146.0 | −0.08991 | 0.98 |
| A1ZM084532_AT | NC0200132 | 4 | 128.7 | −0.08339 | 0.91 |
| A1ZM084532_AT | NC0173476 | 7 | 44.2 | −0.08566 | 0.94 |
| A1ZM084532_AT | NC0199896 | 8 | 96.4 | 0.10085 | 1.10 |
| A1ZM084532_AT | NC0173350 | 8 | 104.0 | 0.08573 | 0.94 |
| A1ZM084532_AT | NC0173530 | 9 | 78.1 | 0.10632 | 1.16 |
| A1ZM084532_AT | NC0199722 | 9 | 79.6 | 0.09771 | 1.07 |
| A1ZM085633_AT | NC0144205 | 1 | 38.5 | −0.07886 | 0.94 |
| A1ZM085633_AT | NC0029053 | 1 | 88.1 | 0.08797 | 1.05 |
| A1ZM085633_AT | NC0174066 | 3 | 168.4 | 0.08763 | 1.04 |
| A1ZM085633_AT | NC0190159 | 3 | 168.4 | 0.08762 | 1.04 |
| A1ZM085633_AT | NC0199442 | 5 | 22.4 | −0.07712 | 0.92 |
| A1ZM085633_AT | NC0040322 | 7 | 64.5 | 0.08921 | 1.06 |
| A1ZM085633_AT | NC0042930 | 7 | 64.5 | 0.07903 | 0.94 |
| A1ZM085633_AT | NC0199312 | 7 | 65.9 | 0.08498 | 1.01 |
| A1ZM085633_AT | NC0199313 | 8 | 89.3 | −0.07726 | 0.92 |
| A1ZM085633_AT | NC0199896 | 8 | 96.4 | 0.10652 | 1.27 |
| A1ZM085633_AT | NC0157736 | 9 | 56.6 | 0.08231 | 0.98 |
| A1ZM085633_AT | NC0200122 | 9 | 56.6 | −0.08231 | 0.98 |
| A1ZM085633_AT | NC0107256 | 9 | 68.5 | −0.07854 | 0.93 |
| A1ZM085633_AT | NC0173530 | 9 | 78.1 | 0.07965 | 0.95 |
| A1ZM085633_AT | NC0199722 | 9 | 79.6 | 0.07739 | 0.92 |
| A1ZM085633_AT | NC0173818 | 10 | 65.3 | 0.08476 | 1.01 |

Next, by including markers proximal to probesets and markers proximal to eQTL, both probesets and eQTL were placed on the same map, and the cis or trans orientation of eQTL relative to the probesets was determined (Table 5). A cis orientation was declared if the marker proximal to the probeset and the marker proximal to the eQTL were one and the same. In Table 5, a value of 1 for the cis match variable indicates this condition. A value of 0 indicates trans orientation. The one-to-one marker match rule for cis orientation has, as a result, a declaration of trans orientation of some eQTL in close proximity to the putative probeset locus. Alternatively, one could have declared cis orientation if the proximal probe marker and proximal eQTL marker were located within the limits of some arbitrarily prescribed map distance.

TABLE 5
Map positions of markers proximal to probesets significantly associated with
yield and markers proximal to expression QTL. A cis match value equal to 1 indicates
that the marker proximal to an eQTL is also proximal to a probeset significantly
associated with yield.

| | | | | | | | | Proximal | Proximal |
|---|---|---|---|---|---|---|---|---|---|
| | | | | | | Proximal | | Expression | Expression |
| | | | Proximal | Proximal | | Expression | | QTL | QTL |
| | Proximal | | Probeset | Expression | | QTL | | Marker | Marker |
| | Probeset | | Marker | QTL | | Marker | Cis | Regression | Regression |
| Probeset | Marker | Chr | Position | Marker | Chr | Position | match | Coefficient | VIP |
| A1ZM000016_S_AT | NC0173951 | 1 | 229.4 | NC0019524 | 1 | 5.8 | 0 | −0.05103 | 0.87 |
| A1ZM000016_S_AT | NC0173951 | 1 | 229.4 | NC0173400 | 1 | 32.2 | 0 | 0.05155 | 0.88 |
| A1ZM000016_S_AT | NC0173951 | 1 | 229.4 | NC0043266 | 1 | 71.3 | 0 | −0.05207 | 0.89 |
| A1ZM000016_S_AT | NC0173951 | 1 | 229.4 | NC0032745 | 1 | 94.5 | 0 | −0.06021 | 1.03 |
| A1ZM000016_S_AT | NC0173951 | 1 | 229.4 | NC0199760 | 1 | 179.1 | 0 | −0.05508 | 0.94 |
| A1ZM000016_S_AT | NC0173951 | 1 | 229.4 | NC0015937 | 1 | 197.7 | 0 | 0.05624 | 0.96 |
| A1ZM000016_S_AT | NC0173951 | 1 | 229.4 | NC0173395 | 1 | 200.1 | 0 | −0.05881 | 1.00 |
| A1ZM000016_S_AT | NC0173951 | 1 | 229.4 | NC0196534 | 1 | 201.9 | 0 | −0.05690 | 0.97 |
| A1ZM000016_S_AT | NC0173951 | 1 | 229.4 | NC0200158 | 1 | 205.1 | 0 | 0.05602 | 0.95 |
| A1ZM000016_S_AT | NC0173951 | 1 | 229.4 | NC0173951 | 1 | 229.4 | 1 | 0.08782 | 1.50 |
| A1ZM000016_S_AT | NC0173951 | 1 | 229.4 | NC0200048 | 1 | 230.2 | 0 | −0.06664 | 1.14 |
| A1ZM000016_S_AT | NC0173951 | 1 | 229.4 | NC0200138 | 1 | 238.1 | 0 | −0.05245 | 0.89 |
| A1ZM000016_S_AT | NC0173951 | 1 | 229.4 | NC0199954 | 2 | 54.1 | 0 | −0.04962 | 0.85 |
| A1ZM000016_S_AT | NC0173951 | 1 | 229.4 | NC0173900 | 6 | 38.4 | 0 | −0.06913 | 1.18 |
| A1ZM000016_S_AT | NC0173951 | 1 | 229.4 | NC0194397 | 6 | 38.4 | 0 | −0.05757 | 0.98 |
| A1ZM000016_S_AT | NC0173951 | 1 | 229.4 | NC0199422 | 6 | 41.1 | 0 | 0.05083 | 0.87 |
| A1ZM000016_S_AT | NC0173951 | 1 | 229.4 | NC0173500 | 6 | 136 | 0 | 0.05870 | 1.00 |
| A1ZM000016_S_AT | NC0173951 | 1 | 229.4 | NC0158250 | 7 | 24.3 | 0 | 0.06448 | 1.10 |
| A1ZM000016_S_AT | NC0173951 | 1 | 229.4 | NC0199331 | 7 | 173.1 | 0 | 0.05440 | 0.93 |
| A1ZM000016_S_AT | NC0173951 | 1 | 229.4 | NC0000129 | 8 | 16.5 | 0 | −0.05977 | 1.02 |
| A1ZM000016_S_AT | NC0173951 | 1 | 229.4 | NC0199387 | 9 | 33 | 0 | −0.06315 | 1.08 |
| A1ZM000016_S_AT | NC0173951 | 1 | 229.4 | NC0200107 | 9 | 65.9 | 0 | −0.04975 | 0.85 |
| A1ZM000016_S_AT | NC0173951 | 1 | 229.4 | NC0199722 | 9 | 79.6 | 0 | 0.05043 | 0.86 |
| A1ZM000016_S_AT | NC0173951 | 1 | 229.4 | NC0014240 | 9 | 103.5 | 0 | −0.05125 | 0.87 |
| A1ZM000016_S_AT | NC0173951 | 1 | 229.4 | NC0158122 | 10 | 36.7 | 0 | −0.06176 | 1.05 |
| A1ZM000016_S_AT | NC0173951 | 1 | 229.4 | NC0199810 | 10 | 48.3 | 0 | 0.05909 | 1.01 |
| A1ZM000016_S_AT | NC0173951 | 1 | 229.4 | NC0173818 | 10 | 65.3 | 0 | 0.07132 | 1.21 |
| A1ZM000016_S_AT | NC0173951 | 1 | 229.4 | NC0011002 | 10 | 66.5 | 0 | −0.05181 | 0.88 |
| A1ZM001187_A_AT | NC0111472 | 8 | 103.3 | NC0144205 | 1 | 38.5 | 0 | 0.03308 | 1.00 |
| A1ZM001187_A_AT | NC0111472 | 8 | 103.3 | NC0199461 | 1 | 176.7 | 0 | 0.02930 | 0.89 |
| A1ZM001187_A_AT | NC0111472 | 8 | 103.3 | NC0173402 | 1 | 217 | 0 | 0.02681 | 0.81 |
| A1ZM001187_A_AT | NC0111472 | 8 | 103.3 | NC0196949 | 2 | 43.4 | 0 | 0.03861 | 1.17 |
| A1ZM001187_A_AT | NC0111472 | 8 | 103.3 | NC0013275 | 2 | 49.7 | 0 | 0.04295 | 1.30 |
| A1ZM001187_A_AT | NC0111472 | 8 | 103.3 | NC0027262 | 2 | 57.3 | 0 | −0.03758 | 1.14 |
| A1ZM001187_A_AT | NC0111472 | 8 | 103.3 | NC0199350 | 2 | 67.8 | 0 | −0.02941 | 0.89 |
| A1ZM001187_A_AT | NC0111472 | 8 | 103.3 | NC0003725 | 2 | 68.4 | 0 | −0.02882 | 0.87 |
| A1ZM001187_A_AT | NC0111472 | 8 | 103.3 | NC0196659 | 2 | 70.4 | 0 | 0.02780 | 0.84 |
| A1ZM001187_A_AT | NC0111472 | 8 | 103.3 | NC0172302 | 2 | 94 | 0 | 0.03140 | 0.95 |
| A1ZM001187_A_AT | NC0111472 | 8 | 103.3 | NC0200126 | 3 | 81.8 | 0 | 0.03069 | 0.93 |
| A1ZM001187_A_AT | NC0111472 | 8 | 103.3 | NC0199803 | 3 | 85.6 | 0 | 0.03232 | 0.98 |
| A1ZM001187_A_AT | NC0111472 | 8 | 103.3 | NC0173436 | 3 | 86 | 0 | 0.03809 | 1.16 |
| A1ZM001187_A_AT | NC0111472 | 8 | 103.3 | NC0000399 | 3 | 121.1 | 0 | 0.03147 | 0.95 |
| A1ZM001187_A_AT | NC0111472 | 8 | 103.3 | NC0015965 | 3 | 140.8 | 0 | 0.02990 | 0.91 |
| A1ZM001187_A_AT | NC0111472 | 8 | 103.3 | NC0112924 | 3 | 145.3 | 0 | 0.03393 | 1.03 |
| A1ZM001187_A_AT | NC0111472 | 8 | 103.3 | NC0067577 | 4 | 59.3 | 0 | −0.03555 | 1.08 |
| A1ZM001187_A_AT | NC0111472 | 8 | 103.3 | NC0199812 | 4 | 77.3 | 0 | 0.02848 | 0.86 |
| A1ZM001187_A_AT | NC0111472 | 8 | 103.3 | NC0171755 | 4 | 77.8 | 0 | 0.02678 | 0.81 |
| A1ZM001187_A_AT | NC0111472 | 8 | 103.3 | NC0173414 | 4 | 78.9 | 0 | −0.03426 | 1.04 |
| A1ZM001187_A_AT | NC0111472 | 8 | 103.3 | NC0028257 | 4 | 81.8 | 0 | −0.02997 | 0.91 |
| A1ZM001187_A_AT | NC0111472 | 8 | 103.3 | NC0199680 | 4 | 81.9 | 0 | −0.02863 | 0.87 |
| A1ZM001187_A_AT | NC0111472 | 8 | 103.3 | NC0199410 | 4 | 82.5 | 0 | 0.02824 | 0.86 |
| A1ZM001187_A_AT | NC0111472 | 8 | 103.3 | NC0200151 | 4 | 84.4 | 0 | 0.03407 | 1.03 |
| A1ZM001187_A_AT | NC0111472 | 8 | 103.3 | NC0200083 | 4 | 84.9 | 0 | −0.02825 | 0.86 |
| A1ZM001187_A_AT | NC0111472 | 8 | 103.3 | NC0188018 | 5 | 85.4 | 0 | −0.02661 | 0.81 |
| A1ZM001187_A_AT | NC0111472 | 8 | 103.3 | NC0173452 | 5 | 177.7 | 0 | −0.03523 | 1.07 |
| A1ZM001187_A_AT | NC0111472 | 8 | 103.3 | NC0173900 | 6 | 38.4 | 0 | 0.02777 | 0.84 |
| A1ZM001187_A_AT | NC0111472 | 8 | 103.3 | NC0200165 | 6 | 99.9 | 0 | −0.02946 | 0.89 |
| A1ZM001187_A_AT | NC0111472 | 8 | 103.3 | NC0173876 | 6 | 104.2 | 0 | −0.02854 | 0.87 |
| A1ZM001187_A_AT | NC0111472 | 8 | 103.3 | NC0195223 | 6 | 118.6 | 0 | 0.04081 | 1.24 |
| A1ZM001187_A_AT | NC0111472 | 8 | 103.3 | NC0199409 | 6 | 118.6 | 0 | −0.03531 | 1.07 |
| A1ZM001187_A_AT | NC0111472 | 8 | 103.3 | NC0199958 | 6 | 142 | 0 | 0.03081 | 0.93 |
| A1ZM001187_A_AT | NC0111472 | 8 | 103.3 | NC0199339 | 7 | 150.8 | 0 | −0.03014 | 0.91 |
| A1ZM001187_A_AT | NC0111472 | 8 | 103.3 | NC0173490 | 8 | 59 | 0 | −0.02811 | 0.85 |
| A1ZM001187_A_AT | NC0111472 | 8 | 103.3 | NC0030011 | 8 | 63.1 | 0 | 0.02794 | 0.85 |
| A1ZM001187_A_AT | NC0111472 | 8 | 103.3 | NC0194645 | 8 | 76.5 | 0 | 0.03672 | 1.11 |
| A1ZM001187_A_AT | NC0111472 | 8 | 103.3 | NC0199730 | 8 | 79.6 | 0 | 0.03433 | 1.04 |
| A1ZM001187_A_AT | NC0111472 | 8 | 103.3 | NC0199735 | 8 | 84.4 | 0 | 0.03359 | 1.02 |
| A1ZM001187_A_AT | NC0111472 | 8 | 103.3 | NC0199770 | 8 | 96.2 | 0 | 0.04149 | 1.26 |
| A1ZM001187_A_AT | NC0111472 | 8 | 103.3 | NC0199896 | 8 | 96.4 | 0 | −0.03137 | 0.95 |
| A1ZM001187_A_AT | NC0111472 | 8 | 103.3 | NC0173626 | 8 | 96.8 | 0 | 0.04015 | 1.22 |
| A1ZM001187_A_AT | NC0111472 | 8 | 103.3 | NC0111472 | 8 | 103.3 | 1 | −0.04788 | 1.45 |
| A1ZM001187_A_AT | NC0111472 | 8 | 103.3 | NC0173350 | 8 | 104 | 0 | −0.03880 | 1.18 |
| A1ZM001187_A_AT | NC0111472 | 8 | 103.3 | NC0199563 | 8 | 115.6 | 0 | −0.03140 | 0.95 |
| A1ZM001187_A_AT | NC0111472 | 8 | 103.3 | NC0033526 | 8 | 153.1 | 0 | 0.02738 | 0.83 |
| A1ZM002154_S_AT | NC0199461 | 1 | 176.7 | NC0173334 | 1 | 102.8 | 0 | −0.04914 | 0.91 |
| A1ZM002154_S_AT | NC0199461 | 1 | 176.7 | NC0173484 | 1 | 103.2 | 0 | −0.05543 | 1.02 |
| A1ZM002154_S_AT | NC0199461 | 1 | 176.7 | NC0199391 | 1 | 107.5 | 0 | −0.05486 | 1.01 |
| A1ZM002154_S_AT | NC0199461 | 1 | 176.7 | NC0188455 | 1 | 113.3 | 0 | −0.06776 | 1.25 |
| A1ZM002154_S_AT | NC0199461 | 1 | 176.7 | NC0199986 | 1 | 113.3 | 0 | −0.05715 | 1.06 |
| A1ZM002154_S_AT | NC0199461 | 1 | 176.7 | NC0199461 | 1 | 176.7 | 1 | 0.07007 | 1.30 |
| A1ZM002154_S_AT | NC0199461 | 1 | 176.7 | NC0173951 | 1 | 229.4 | 0 | −0.05173 | 0.96 |
| A1ZM002154_S_AT | NC0199461 | 1 | 176.7 | NC0173409 | 2 | 32.7 | 0 | −0.05030 | 0.93 |
| A1ZM002154_S_AT | NC0199461 | 1 | 176.7 | NC0170324 | 2 | 94 | 0 | 0.04976 | 0.92 |
| A1ZM002154_S_AT | NC0199461 | 1 | 176.7 | NC0174027 | 2 | 99.2 | 0 | −0.05375 | 0.99 |
| A1ZM002154_S_AT | NC0199461 | 1 | 176.7 | NC0199297 | 2 | 100.2 | 0 | −0.05008 | 0.93 |
| A1ZM002154_S_AT | NC0199461 | 1 | 176.7 | NC0199851 | 2 | 165.2 | 0 | −0.04933 | 0.91 |
| A1ZM002154_S_AT | NC0199461 | 1 | 176.7 | NC0033320 | 2 | 173.6 | 0 | 0.05233 | 0.97 |
| A1ZM002154_S_AT | NC0199461 | 1 | 176.7 | NC0051616 | 3 | 40.4 | 0 | −0.05721 | 1.06 |
| A1ZM002154_S_AT | NC0199461 | 1 | 176.7 | NC0199522 | 3 | 90.3 | 0 | 0.05926 | 1.10 |
| A1ZM002154_S_AT | NC0199461 | 1 | 176.7 | NC0112481 | 3 | 183.6 | 0 | −0.06465 | 1.20 |
| A1ZM002154_S_AT | NC0199461 | 1 | 176.7 | NC0170468 | 3 | 216.7 | 0 | 0.04604 | 0.85 |
| A1ZM002154_S_AT | NC0199461 | 1 | 176.7 | NC0036637 | 5 | 100 | 0 | −0.04879 | 0.90 |
| A1ZM002154_S_AT | NC0199461 | 1 | 176.7 | NC0196774 | 6 | 69.4 | 0 | 0.05249 | 0.97 |
| A1ZM002154_S_AT | NC0199461 | 1 | 176.7 | NC0173525 | 6 | 70.2 | 0 | 0.05110 | 0.94 |
| A1ZM002154_S_AT | NC0199461 | 1 | 176.7 | NC0199800 | 7 | 114.5 | 0 | −0.05174 | 0.96 |
| A1ZM002154_S_AT | NC0199461 | 1 | 176.7 | NC0199443 | 8 | 72.9 | 0 | 0.06171 | 1.14 |
| A1ZM002154_S_AT | NC0199461 | 1 | 176.7 | NC0194645 | 8 | 76.5 | 0 | 0.05797 | 1.07 |
| A1ZM002154_S_AT | NC0199461 | 1 | 176.7 | NC0199730 | 8 | 79.6 | 0 | 0.05532 | 1.02 |
| A1ZM002154_S_AT | NC0199461 | 1 | 176.7 | NC0199735 | 8 | 84.4 | 0 | 0.05286 | 0.98 |
| A1ZM002154_S_AT | NC0199461 | 1 | 176.7 | NC0174094 | 8 | 91.1 | 0 | 0.04634 | 0.86 |
| A1ZM002154_S_AT | NC0199461 | 1 | 176.7 | NC0199563 | 8 | 115.6 | 0 | −0.04742 | 0.88 |
| A1ZM002154_S_AT | NC0199461 | 1 | 176.7 | NC0033526 | 8 | 153.1 | 0 | 0.04597 | 0.85 |
| A1ZM002154_S_AT | NC0199461 | 1 | 176.7 | NC0014545 | 8 | 155.1 | 0 | 0.04626 | 0.86 |
| A1ZM002154_S_AT | NC0199461 | 1 | 176.7 | NC0107256 | 9 | 68.5 | 0 | 0.05121 | 0.95 |
| A1ZM002154_S_AT | NC0199461 | 1 | 176.7 | NC0173571 | 9 | 90.4 | 0 | −0.05597 | 1.03 |
| A1ZM002154_S_AT | NC0199461 | 1 | 176.7 | NC0148189 | 9 | 91 | 0 | −0.05635 | 1.04 |
| A1ZM003299_S_AT | NC0013275 | 2 | 49.7 | NC0199461 | 1 | 176.7 | 0 | 0.04694 | 1.00 |
| A1ZM003299_S_AT | NC0013275 | 2 | 49.7 | NC0173409 | 2 | 32.7 | 0 | −0.04268 | 0.91 |
| A1ZM003299_S_AT | NC0013275 | 2 | 49.7 | NC0196949 | 2 | 43.4 | 0 | 0.05657 | 1.20 |
| A1ZM003299_S_AT | NC0013275 | 2 | 49.7 | NC0013275 | 2 | 49.7 | 1 | 0.06505 | 1.38 |
| A1ZM003299_S_AT | NC0013275 | 2 | 49.7 | NC0199346 | 2 | 50.8 | 0 | 0.04034 | 0.86 |
| A1ZM003299_S_AT | NC0013275 | 2 | 49.7 | NC0002630 | 2 | 54.1 | 0 | −0.04810 | 1.02 |
| A1ZM003299_S_AT | NC0013275 | 2 | 49.7 | NC0027262 | 2 | 57.3 | 0 | −0.04482 | 0.95 |
| A1ZM003299_S_AT | NC0013275 | 2 | 49.7 | NC0015965 | 3 | 140.8 | 0 | 0.05244 | 1.11 |
| A1ZM003299_S_AT | NC0013275 | 2 | 49.7 | NC0199794 | 3 | 146 | 0 | 0.05729 | 1.22 |
| A1ZM003299_S_AT | NC0013275 | 2 | 49.7 | NC0171755 | 4 | 77.8 | 0 | 0.04259 | 0.91 |
| A1ZM003299_S_AT | NC0013275 | 2 | 49.7 | NC0199680 | 4 | 81.9 | 0 | −0.04290 | 0.91 |
| A1ZM003299_S_AT | NC0013275 | 2 | 49.7 | NC0199410 | 4 | 82.5 | 0 | 0.04138 | 0.88 |
| A1ZM003299_S_AT | NC0013275 | 2 | 49.7 | NC0199832 | 4 | 84.7 | 0 | 0.04994 | 1.06 |
| A1ZM003299_S_AT | NC0013275 | 2 | 49.7 | NC0200083 | 4 | 84.9 | 0 | −0.04708 | 1.00 |
| A1ZM003299_S_AT | NC0013275 | 2 | 49.7 | NC0199826 | 4 | 190.8 | 0 | 0.04467 | 0.95 |
| A1ZM003299_S_AT | NC0013275 | 2 | 49.7 | NC0199455 | 6 | 73.3 | 0 | 0.04219 | 0.90 |
| A1ZM003299_S_AT | NC0013275 | 2 | 49.7 | NC0173962 | 6 | 96.7 | 0 | 0.04980 | 1.06 |
| A1ZM003299_S_AT | NC0013275 | 2 | 49.7 | NC0194601 | 6 | 143.7 | 0 | −0.04979 | 1.06 |
| A1ZM003299_S_AT | NC0013275 | 2 | 49.7 | NC0199685 | 7 | 28.6 | 0 | 0.04194 | 0.89 |
| A1ZM003299_S_AT | NC0013275 | 2 | 49.7 | NC0199937 | 7 | 31.2 | 0 | 0.04587 | 0.98 |
| A1ZM003299_S_AT | NC0013275 | 2 | 49.7 | NC0174167 | 7 | 58 | 0 | −0.04487 | 0.95 |
| A1ZM003299_S_AT | NC0013275 | 2 | 49.7 | NC0199770 | 8 | 96.2 | 0 | 0.04193 | 0.89 |
| A1ZM003299_S_AT | NC0013275 | 2 | 49.7 | NC0173626 | 8 | 96.8 | 0 | 0.04549 | 0.97 |
| A1ZM003299_S_AT | NC0013275 | 2 | 49.7 | NC0111472 | 8 | 103.3 | 0 | −0.04147 | 0.88 |
| A1ZM003299_S_AT | NC0013275 | 2 | 49.7 | NC0199722 | 9 | 79.6 | 0 | −0.04289 | 0.91 |
| A1ZM003299_S_AT | NC0013275 | 2 | 49.7 | NC0148189 | 9 | 91 | 0 | −0.04498 | 0.96 |
| A1ZM004376_S_AT | NC0035106 | 8 | 151.4 | NC0173334 | 1 | 102.8 | 0 | −0.05260 | 0.92 |
| A1ZM004376_S_AT | NC0035106 | 8 | 151.4 | NC0173484 | 1 | 103.2 | 0 | −0.05077 | 0.89 |
| A1ZM004376_S_AT | NC0035106 | 8 | 151.4 | NC0199986 | 1 | 113.3 | 0 | −0.05762 | 1.01 |
| A1ZM004376_S_AT | NC0035106 | 8 | 151.4 | NC0199350 | 2 | 67.8 | 0 | −0.05746 | 1.00 |
| A1ZM004376_S_AT | NC0035106 | 8 | 151.4 | NC0196659 | 2 | 70.4 | 0 | 0.05178 | 0.90 |
| A1ZM004376_S_AT | NC0035106 | 8 | 151.4 | NC0000399 | 3 | 121.1 | 0 | 0.05486 | 0.96 |
| A1ZM004376_S_AT | NC0035106 | 8 | 151.4 | NC0190032 | 3 | 147.8 | 0 | 0.05218 | 0.91 |
| A1ZM004376_S_AT | NC0035106 | 8 | 151.4 | NC0018073 | 3 | 201.6 | 0 | 0.05056 | 0.88 |
| A1ZM004376_S_AT | NC0035106 | 8 | 151.4 | NC0199978 | 4 | 70.6 | 0 | 0.06506 | 1.13 |
| A1ZM004376_S_AT | NC0035106 | 8 | 151.4 | NC0199812 | 4 | 77.3 | 0 | 0.06070 | 1.06 |
| A1ZM004376_S_AT | NC0035106 | 8 | 151.4 | NC0171755 | 4 | 77.8 | 0 | 0.05270 | 0.92 |
| A1ZM004376_S_AT | NC0035106 | 8 | 151.4 | NC0173414 | 4 | 78.9 | 0 | −0.04993 | 0.87 |
| A1ZM004376_S_AT | NC0035106 | 8 | 151.4 | NC0199603 | 4 | 79.8 | 0 | −0.05651 | 0.99 |
| A1ZM004376_S_AT | NC0035106 | 8 | 151.4 | NC0200083 | 4 | 84.9 | 0 | −0.05731 | 1.00 |
| A1ZM004376_S_AT | NC0035106 | 8 | 151.4 | NC0189286 | 4 | 177 | 0 | 0.05243 | 0.91 |
| A1ZM004376_S_AT | NC0035106 | 8 | 151.4 | NC0147215 | 4 | 179.6 | 0 | −0.06321 | 1.10 |
| A1ZM004376_S_AT | NC0035106 | 8 | 151.4 | NC0194601 | 6 | 143.7 | 0 | −0.06049 | 1.06 |
| A1ZM004376_S_AT | NC0035106 | 8 | 151.4 | NC0173584 | 7 | 8.5 | 0 | −0.05920 | 1.03 |
| A1ZM004376_S_AT | NC0035106 | 8 | 151.4 | NC0174551 | 7 | 11.2 | 0 | 0.05404 | 0.94 |
| A1ZM004376_S_AT | NC0035106 | 8 | 151.4 | NC0199805 | 8 | 51.7 | 0 | 0.05602 | 0.98 |
| A1ZM004376_S_AT | NC0035106 | 8 | 151.4 | NC0199563 | 8 | 115.6 | 0 | −0.05861 | 1.02 |
| A1ZM004376_S_AT | NC0035106 | 8 | 151.4 | NC0035106 | 8 | 151.4 | 1 | −0.07551 | 1.32 |
| A1ZM004376_S_AT | NC0035106 | 8 | 151.4 | NC0033526 | 8 | 153.1 | 0 | 0.05998 | 1.05 |
| A1ZM004376_S_AT | NC0035106 | 8 | 151.4 | NC0014545 | 8 | 155.1 | 0 | 0.06660 | 1.16 |
| A1ZM004376_S_AT | NC0035106 | 8 | 151.4 | NC0199880 | 8 | 161.8 | 0 | 0.04940 | 0.86 |
| A1ZM005469_AT | NC0070876 | 1 | 19.7 | NC0039304 | 1 | 19.7 | 0 | −0.06679 | 0.99 |
| A1ZM005469_AT | NC0070876 | 1 | 19.7 | NC0070876 | 1 | 19.7 | 1 | −0.09737 | 1.45 |
| A1ZM005469_AT | NC0070876 | 1 | 19.7 | NC0173555 | 1 | 22.9 | 0 | 0.09283 | 1.38 |
| A1ZM005469_AT | NC0070876 | 1 | 19.7 | NC0173332 | 1 | 33.3 | 0 | −0.06261 | 0.93 |
| A1ZM005469_AT | NC0070876 | 1 | 19.7 | NC0157397 | 1 | 87.7 | 0 | 0.05918 | 0.88 |
| A1ZM005469_AT | NC0070876 | 1 | 19.7 | NC0171154 | 1 | 88.1 | 0 | −0.05657 | 0.84 |
| A1ZM005469_AT | NC0070876 | 1 | 19.7 | NC0199317 | 2 | 138.2 | 0 | −0.06102 | 0.91 |
| A1ZM005469_AT | NC0070876 | 1 | 19.7 | NC0003536 | 3 | 30.9 | 0 | 0.06132 | 0.91 |
| A1ZM005469_AT | NC0070876 | 1 | 19.7 | NC0010671 | 4 | 63.7 | 0 | −0.05782 | 0.86 |
| A1ZM005469_AT | NC0070876 | 1 | 19.7 | NC0173568 | 4 | 150.5 | 0 | −0.06954 | 1.03 |
| A1ZM005469_AT | NC0070876 | 1 | 19.7 | NC0187978 | 7 | 165.7 | 0 | −0.06223 | 0.92 |
| A1ZM005469_AT | NC0070876 | 1 | 19.7 | NC0199331 | 7 | 173.1 | 0 | −0.05680 | 0.84 |
| A1ZM005469_AT | NC0070876 | 1 | 19.7 | NC0199735 | 8 | 84.4 | 0 | 0.06557 | 0.97 |
| A1ZM005469_AT | NC0070876 | 1 | 19.7 | NC0199440 | 8 | 87.9 | 0 | −0.07186 | 1.07 |
| A1ZM005469_AT | NC0070876 | 1 | 19.7 | NC0199313 | 8 | 89.3 | 0 | 0.07323 | 1.09 |
| A1ZM005469_AT | NC0070876 | 1 | 19.7 | NC0199860 | 8 | 93.9 | 0 | 0.06022 | 0.89 |
| A1ZM005469_AT | NC0070876 | 1 | 19.7 | NC0199896 | 8 | 96.4 | 0 | −0.07520 | 1.12 |
| A1ZM005469_AT | NC0070876 | 1 | 19.7 | NC0199563 | 8 | 115.6 | 0 | −0.05771 | 0.86 |
| A1ZM005469_AT | NC0070876 | 1 | 19.7 | NC0199525 | 9 | 90.9 | 0 | −0.05799 | 0.86 |
| A1ZM005469_AT | NC0070876 | 1 | 19.7 | NC0148189 | 9 | 91 | 0 | −0.06203 | 0.92 |
| A1ZM006628_A_AT | NC0199443 | 8 | 72.9 | NC0035426 | 1 | 34.5 | 0 | 0.03411 | 0.86 |
| A1ZM006628_A_AT | NC0199443 | 8 | 72.9 | NC0170811 | 1 | 153.9 | 0 | 0.04197 | 1.06 |
| A1ZM006628_A_AT | NC0199443 | 8 | 72.9 | NC0173449 | 1 | 154.4 | 0 | −0.04329 | 1.10 |
| A1ZM006628_A_AT | NC0199443 | 8 | 72.9 | NC0009148 | 1 | 213 | 0 | −0.03363 | 0.85 |
| A1ZM006628_A_AT | NC0199443 | 8 | 72.9 | NC0112831 | 2 | 9 | 0 | 0.03265 | 0.83 |
| A1ZM006628_A_AT | NC0199443 | 8 | 72.9 | NC0199954 | 2 | 54.1 | 0 | −0.03498 | 0.88 |
| A1ZM006628_A_AT | NC0199443 | 8 | 72.9 | NC0200081 | 3 | 115.9 | 0 | −0.04317 | 1.09 |
| A1ZM006628_A_AT | NC0199443 | 8 | 72.9 | NC0112924 | 3 | 145.3 | 0 | −0.03240 | 0.82 |
| A1ZM006628_A_AT | NC0199443 | 8 | 72.9 | NC0112481 | 3 | 183.6 | 0 | 0.03312 | 0.84 |
| A1ZM006628_A_AT | NC0199443 | 8 | 72.9 | NC0199293 | 5 | 62.9 | 0 | −0.04181 | 1.06 |
| A1ZM006628_A_AT | NC0199443 | 8 | 72.9 | NC0173716 | 5 | 81.5 | 0 | −0.03877 | 0.98 |
| A1ZM006628 A_AT | NC0199443 | 8 | 72.9 | NC0194693 | 5 | 89.9 | 0 | −0.04439 | 1.12 |
| A1ZM006628_A_AT | NC0199443 | 8 | 72.9 | NC0078090 | 5 | 141.8 | 0 | −0.03512 | 0.89 |
| A1ZM006628_A_AT | NC0199443 | 8 | 72.9 | NC0173396 | 5 | 143.1 | 0 | −0.03442 | 0.87 |
| A1ZM006628_A_AT | NC0199443 | 8 | 72.9 | NC0042059 | 5 | 143.2 | 0 | −0.03846 | 0.97 |
| A1ZM006628_A_AT | NC0199443 | 8 | 72.9 | NC0173843 | 5 | 143.2 | 0 | 0.03531 | 0.89 |
| A1ZM006628_A_AT | NC0199443 | 8 | 72.9 | NC0020696 | 6 | 38.5 | 0 | 0.03686 | 0.93 |
| A1ZM006628_A_AT | NC0199443 | 8 | 72.9 | NC0199750 | 6 | 81.6 | 0 | 0.03475 | 0.88 |
| A1ZM006628_A_AT | NC0199443 | 8 | 72.9 | NC0013445 | 6 | 131.2 | 0 | −0.03456 | 0.87 |
| A1ZM006628_A_AT | NC0199443 | 8 | 72.9 | NC0199331 | 7 | 173.1 | 0 | 0.03451 | 0.87 |
| A1ZM006628_A_AT | NC0199443 | 8 | 72.9 | NC0079084 | 8 | 65.2 | 0 | 0.04295 | 1.09 |
| A1ZM006628_A_AT | NC0199443 | 8 | 72.9 | NC0200128 | 8 | 65.7 | 0 | −0.04161 | 1.05 |
| A1ZM006628_A_AT | NC0199443 | 8 | 72.9 | NC0171070 | 8 | 70 | 0 | −0.04686 | 1.19 |
| A1ZM006628_A_AT | NC0199443 | 8 | 72.9 | NC0173531 | 8 | 70 | 0 | 0.03235 | 0.82 |
| A1ZM006628_A_AT | NC0199443 | 8 | 72.9 | NC0199443 | 8 | 72.9 | 1 | −0.05684 | 1.44 |
| A1ZM006628_A_AT | NC0199443 | 8 | 72.9 | NC0194645 | 8 | 76.5 | 0 | −0.04259 | 1.08 |
| A1ZM006628_A_AT | NC0199443 | 8 | 72.9 | NC0199730 | 8 | 79.6 | 0 | −0.04894 | 1.24 |
| A1ZM006628_A_AT | NC0199443 | 8 | 72.9 | NC0199735 | 8 | 84.4 | 0 | −0.03664 | 0.93 |
| A1ZM006628_A_AT | NC0199443 | 8 | 72.9 | NC0199770 | 8 | 96.2 | 0 | −0.03635 | 0.92 |
| A1ZM006628_A_AT | NC0199443 | 8 | 72.9 | NC0199896 | 8 | 96.4 | 0 | 0.05208 | 1.32 |
| A1ZM006628_A_AT | NC0199443 | 8 | 72.9 | NC0173626 | 8 | 96.8 | 0 | −0.04195 | 1.06 |
| A1ZM006628_A_AT | NC0199443 | 8 | 72.9 | NC0111472 | 8 | 103.3 | 0 | 0.03729 | 0.94 |
| A1ZM006628_A_AT | NC0199443 | 8 | 72.9 | NC0035106 | 8 | 151.4 | 0 | 0.03548 | 0.90 |
| A1ZM006628_A_AT | NC0199443 | 8 | 72.9 | NC0033526 | 8 | 153.1 | 0 | −0.03808 | 0.96 |
| A1ZM006628_A_AT | NC0199443 | 8 | 72.9 | NC0199956 | 8 | 169.9 | 0 | 0.04077 | 1.03 |
| A1ZM006707_AT | NC0112924 | 3 | 145.3 | NC0199703 | 1 | 26.9 | 0 | −0.05681 | 0.95 |
| A1ZM006707_AT | NC0112924 | 3 | 145.3 | NC0029053 | 1 | 88.1 | 0 | −0.06064 | 1.02 |
| A1ZM006707_AT | NC0112924 | 3 | 145.3 | NC0199461 | 1 | 176.7 | 0 | 0.05488 | 0.92 |
| A1ZM006707_AT | NC0112924 | 3 | 145.3 | NC0013275 | 2 | 49.7 | 0 | 0.06825 | 1.15 |
| A1ZM006707_AT | NC0112924 | 3 | 145.3 | NC0199346 | 2 | 50.8 | 0 | 0.05387 | 0.91 |
| A1ZM006707_AT | NC0112924 | 3 | 145.3 | NC0002630 | 2 | 54.1 | 0 | −0.05599 | 0.94 |
| A1ZM006707_AT | NC0112924 | 3 | 145.3 | NC0199803 | 3 | 85.6 | 0 | 0.05449 | 0.92 |
| A1ZM006707_AT | NC0112924 | 3 | 145.3 | NC0009470 | 3 | 88 | 0 | −0.05683 | 0.96 |
| A1ZM006707_AT | NC0112924 | 3 | 145.3 | NC0199522 | 3 | 90.3 | 0 | 0.05701 | 0.96 |
| A1ZM006707_AT | NC0112924 | 3 | 145.3 | NC0112924 | 3 | 145.3 | 1 | 0.07244 | 1.22 |
| A1ZM006707_AT | NC0112924 | 3 | 145.3 | NC0199826 | 4 | 190.8 | 0 | 0.05399 | 0.91 |
| A1ZM006707_AT | NC0112924 | 3 | 145.3 | NC0200106 | 5 | 124.4 | 0 | 0.05704 | 0.96 |
| A1ZM006707_AT | NC0112924 | 3 | 145.3 | NC0107877 | 5 | 132.7 | 0 | −0.06609 | 1.11 |
| A1ZM006707_AT | NC0112924 | 3 | 145.3 | NC0200064 | 7 | 65.3 | 0 | −0.05462 | 0.92 |
| A1ZM006707_AT | NC0112924 | 3 | 145.3 | NC0000129 | 8 | 16.5 | 0 | 0.05852 | 0.98 |
| A1ZM006707_AT | NC0112924 | 3 | 145.3 | NC0173490 | 8 | 59 | 0 | −0.05517 | 0.93 |
| A1ZM006707_AT | NC0112924 | 3 | 145.3 | NC0199735 | 8 | 84.4 | 0 | 0.05578 | 0.94 |
| A1ZM006707_AT | NC0112924 | 3 | 145.3 | NC0199770 | 8 | 96.2 | 0 | 0.06662 | 1.12 |
| A1ZM006707_AT | NC0112924 | 3 | 145.3 | NC0173626 | 8 | 96.8 | 0 | 0.05897 | 0.99 |
| A1ZM006707_AT | NC0112924 | 3 | 145.3 | NC0111472 | 8 | 103.3 | 0 | −0.06684 | 1.12 |
| A1ZM006707_AT | NC0112924 | 3 | 145.3 | NC0199563 | 8 | 115.6 | 0 | −0.05409 | 0.91 |
| A1ZM006707_AT | NC0112924 | 3 | 145.3 | NC0020872 | 9 | 76.3 | 0 | 0.05629 | 0.95 |
| A1ZM006707_AT | NC0112924 | 3 | 145.3 | NC0199722 | 9 | 79.6 | 0 | −0.06717 | 1.13 |
| A1ZM011510_S_AT | NC0199741 | 3 | 125.7 | NC0036726 | 1 | 35.2 | 0 | −0.03738 | 0.85 |
| A1ZM011510_S_AT | NC0199741 | 3 | 125.7 | NC0199491 | 1 | 183.9 | 0 | 0.03616 | 0.82 |
| A1ZM011510_S_AT | NC0199741 | 3 | 125.7 | NC0196949 | 2 | 43.4 | 0 | −0.05180 | 1.18 |
| A1ZM011510_S_AT | NC0199741 | 3 | 125.7 | NC0013275 | 2 | 49.7 | 0 | −0.04918 | 1.12 |
| A1ZM011510_S_AT | NC0199741 | 3 | 125.7 | NC0027262 | 2 | 57.3 | 0 | 0.03790 | 0.86 |
| A1ZM011510_S_AT | NC0199741 | 3 | 125.7 | NC0173436 | 3 | 86 | 0 | −0.03940 | 0.90 |
| A1ZM011510_S_AT | NC0199741 | 3 | 125.7 | NC0000399 | 3 | 121.1 | 0 | −0.04140 | 0.94 |
| A1ZM011510_S_AT | NC0199741 | 3 | 125.7 | NC0199741 | 3 | 125.7 | 1 | −0.06208 | 1.41 |
| A1ZM011510_S_AT | NC0199741 | 3 | 125.7 | NC0112924 | 3 | 145.3 | 0 | −0.04940 | 1.12 |
| A1ZM011510_S_AT | NC0199741 | 3 | 125.7 | NC0199794 | 3 | 146 | 0 | −0.03949 | 0.90 |
| A1ZM011510_S_AT | NC0199741 | 3 | 125.7 | NC0000189 | 3 | 215.3 | 0 | 0.03530 | 0.80 |
| A1ZM011510_S_AT | NC0199741 | 3 | 125.7 | NC0193457 | 3 | 216.6 | 0 | 0.03651 | 0.83 |
| A1ZM011510_S_AT | NC0199741 | 3 | 125.7 | NC0024368 | 4 | 162.6 | 0 | −0.03711 | 0.84 |
| A1ZM011510_S_AT | NC0199741 | 3 | 125.7 | NC0195223 | 6 | 118.6 | 0 | −0.04211 | 0.96 |
| A1ZM011510_S_AT | NC0199741 | 3 | 125.7 | NC0199409 | 6 | 118.6 | 0 | 0.03745 | 0.85 |
| A1ZM011510_S_AT | NC0199741 | 3 | 125.7 | NC0194645 | 8 | 76.5 | 0 | −0.04861 | 1.11 |
| A1ZM011510_S_AT | NC0199741 | 3 | 125.7 | NC0199730 | 8 | 79.6 | 0 | −0.05117 | 1.16 |
| A1ZM011510_S_AT | NC0199741 | 3 | 125.7 | NC0199770 | 8 | 96.2 | 0 | −0.04038 | 0.92 |
| A1ZM011510_S_AT | NC0199741 | 3 | 125.7 | NC0199896 | 8 | 96.4 | 0 | 0.03841 | 0.87 |
| A1ZM011510_S_AT | NC0199741 | 3 | 125.7 | NC0111472 | 8 | 103.3 | 0 | 0.03984 | 0.91 |
| A1ZM011510_S_AT | NC0199741 | 3 | 125.7 | NC0035106 | 8 | 151.4 | 0 | 0.04193 | 0.95 |
| A1ZM011510_S_AT | NC0199741 | 3 | 125.7 | NC0029744 | 9 | 67.4 | 0 | 0.04876 | 1.11 |
| A1ZM011510_S_AT | NC0199741 | 3 | 125.7 | NC0173953 | 9 | 67.4 | 0 | −0.04219 | 0.96 |
| A1ZM011510_S_AT | NC0199741 | 3 | 125.7 | NC0173530 | 9 | 78.1 | 0 | 0.03813 | 0.87 |
| A1ZM011510_S_AT | NC0199741 | 3 | 125.7 | NC0199722 | 9 | 79.6 | 0 | 0.04372 | 0.99 |
| A1ZM011510_S_AT | NC0199741 | 3 | 125.7 | NC0030512 | 9 | 103.6 | 0 | −0.03620 | 0.82 |
| A1ZM011510_S_AT | NC0199741 | 3 | 125.7 | NC0200144 | 9 | 110.4 | 0 | 0.05930 | 1.35 |
| A1ZM011510_S_AT | NC0199741 | 3 | 125.7 | NC0051922 | 9 | 110.5 | 0 | 0.04828 | 1.10 |
| A1ZM011510_S_AT | NC0199741 | 3 | 125.7 | NC0029024 | 9 | 116.9 | 0 | 0.04991 | 1.14 |
| A1ZM011510_S_AT | NC0199741 | 3 | 125.7 | NC0199898 | 9 | 119.2 | 0 | 0.04984 | 1.13 |
| A1ZM011510_S_AT | NC0199741 | 3 | 125.7 | NC0199686 | 10 | 49.1 | 0 | −0.03610 | 0.82 |
| A1ZM011510_S_AT | NC0199741 | 3 | 125.7 | NC0199366 | 10 | 51.6 | 0 | −0.03634 | 0.83 |
| A1ZM011510_S_AT | NC0199741 | 3 | 125.7 | NC0053108 | 10 | 61.2 | 0 | −0.04216 | 0.96 |
| A1ZM011510_S_AT | NC0199741 | 3 | 125.7 | NC0011002 | 10 | 66.5 | 0 | −0.05247 | 1.19 |
| A1ZM015898_AT | NC0199735 | 8 | 84.4 | NC0029053 | 1 | 88.1 | 0 | 0.05599 | 0.84 |
| A1ZM015898_AT | NC0199735 | 8 | 84.4 | NC0013275 | 2 | 49.7 | 0 | −0.06396 | 0.96 |
| A1ZM015898_AT | NC0199735 | 8 | 84.4 | NC0199346 | 2 | 50.8 | 0 | −0.06045 | 0.90 |
| A1ZM015898_AT | NC0199735 | 8 | 84.4 | NC0199480 | 6 | 99.9 | 0 | 0.05898 | 0.88 |
| A1ZM015898_AT | NC0199735 | 8 | 84.4 | NC0199339 | 7 | 150.8 | 0 | 0.06161 | 0.92 |
| A1ZM015898_AT | NC0199735 | 8 | 84.4 | NC0199730 | 8 | 79.6 | 0 | −0.05413 | 0.81 |
| A1ZM015898_AT | NC0199735 | 8 | 84.4 | NC0199735 | 8 | 84.4 | 1 | −0.08786 | 1.31 |
| A1ZM015898_AT | NC0199735 | 8 | 84.4 | NC0199440 | 8 | 87.9 | 0 | 0.06348 | 0.95 |
| A1ZM015898_AT | NC0199735 | 8 | 84.4 | NC0199313 | 8 | 89.3 | 0 | −0.05530 | 0.83 |
| A1ZM015898_AT | NC0199735 | 8 | 84.4 | NC0145457 | 8 | 91.1 | 0 | 0.06383 | 0.95 |
| A1ZM015898_AT | NC0199735 | 8 | 84.4 | NC0199860 | 8 | 93.9 | 0 | −0.06709 | 1.00 |
| A1ZM015898_AT | NC0199735 | 8 | 84.4 | NC0151542 | 8 | 96.2 | 0 | 0.06277 | 0.94 |
| A1ZM015898_AT | NC0199735 | 8 | 84.4 | NC0199770 | 8 | 96.2 | 0 | −0.08183 | 1.22 |
| A1ZM015898_AT | NC0199735 | 8 | 84.4 | NC0199896 | 8 | 96.4 | 0 | 0.06821 | 1.02 |
| A1ZM015898_AT | NC0199735 | 8 | 84.4 | NC0173626 | 8 | 96.8 | 0 | −0.08382 | 1.25 |
| A1ZM015898_AT | NC0199735 | 8 | 84.4 | NC0111472 | 8 | 103.3 | 0 | 0.06265 | 0.94 |
| A1ZM015898_AT | NC0199735 | 8 | 84.4 | NC0157736 | 9 | 56.6 | 0 | 0.06973 | 1.04 |
| A1ZM015898_AT | NC0199735 | 8 | 84.4 | NC0200122 | 9 | 56.6 | 0 | −0.06973 | 1.04 |
| A1ZM017348_S_AT | NC0033526 | 8 | 153.1 | NC0199461 | 1 | 176.7 | 0 | −0.04076 | 0.99 |
| A1ZM017348_S_AT | NC0033526 | 8 | 153.1 | NC0199760 | 1 | 179.1 | 0 | −0.03946 | 0.96 |
| A1ZM017348_S_AT | NC0033526 | 8 | 153.1 | NC0200153 | 1 | 182.8 | 0 | 0.03759 | 0.91 |
| A1ZM017348_S_AT | NC0033526 | 8 | 153.1 | NC0080029 | 2 | 33.1 | 0 | −0.03741 | 0.91 |
| A1ZM017348_S_AT | NC0033526 | 8 | 153.1 | NC0196949 | 2 | 43.4 | 0 | −0.04274 | 1.04 |
| A1ZM017348_S_AT | NC0033526 | 8 | 153.1 | NC0013275 | 2 | 49.7 | 0 | −0.03625 | 0.88 |
| A1ZM017348_S_AT | NC0033526 | 8 | 153.1 | NC0199350 | 2 | 67.8 | 0 | 0.03362 | 0.82 |
| A1ZM017348_S_AT | NC0033526 | 8 | 153.1 | NC0200081 | 3 | 115.9 | 0 | −0.03753 | 0.91 |
| A1ZM017348_S_AT | NC0033526 | 8 | 153.1 | NC0000399 | 3 | 121.1 | 0 | −0.05109 | 1.24 |
| A1ZM017348_S_AT | NC0033526 | 8 | 153.1 | NC0199741 | 3 | 125.7 | 0 | −0.04216 | 1.02 |
| A1ZM017348_S_AT | NC0033526 | 8 | 153.1 | NC0015965 | 3 | 140.8 | 0 | −0.03767 | 0.92 |
| A1ZM017348_S_AT | NC0033526 | 8 | 153.1 | NC0112924 | 3 | 145.3 | 0 | −0.04686 | 1.14 |
| A1ZM017348_S_AT | NC0033526 | 8 | 153.1 | NC0199794 | 3 | 146 | 0 | −0.03333 | 0.81 |
| A1ZM017348_S_AT | NC0033526 | 8 | 153.1 | NC0190032 | 3 | 147.8 | 0 | −0.03545 | 0.86 |
| A1ZM017348_S_AT | NC0033526 | 8 | 153.1 | NC0199913 | 3 | 152.8 | 0 | −0.03557 | 0.86 |
| A1ZM017348_S_AT | NC0033526 | 8 | 153.1 | NC0035568 | 3 | 166.8 | 0 | 0.03586 | 0.87 |
| A1ZM017348_S_AT | NC0033526 | 8 | 153.1 | NC0174066 | 3 | 168.4 | 0 | 0.03532 | 0.86 |
| A1ZM017348_S_AT | NC0033526 | 8 | 153.1 | NC0199675 | 5 | 45.9 | 0 | −0.03563 | 0.87 |
| A1ZM017348_S_AT | NC0033526 | 8 | 153.1 | NC0199293 | 5 | 62.9 | 0 | −0.04994 | 1.21 |
| A1ZM017348_S_AT | NC0033526 | 8 | 153.1 | NC0199447 | 5 | 80.6 | 0 | −0.05312 | 1.29 |
| A1ZM017348_S_AT | NC0033526 | 8 | 153.1 | NC0173900 | 6 | 38.4 | 0 | −0.03339 | 0.81 |
| A1ZM017348_S_AT | NC0033526 | 8 | 153.1 | NC0020696 | 6 | 38.5 | 0 | 0.03766 | 0.92 |
| A1ZM017348_S_AT | NC0033526 | 8 | 153.1 | NC0029612 | 6 | 57.8 | 0 | −0.03349 | 0.81 |
| A1ZM017348_S_AT | NC0033526 | 8 | 153.1 | NC0200165 | 6 | 99.9 | 0 | 0.03400 | 0.83 |
| A1ZM017348_S_AT | NC0033526 | 8 | 153.1 | NC0195223 | 6 | 118.6 | 0 | −0.03929 | 0.96 |
| A1ZM017348_S_AT | NC0033526 | 8 | 153.1 | NC0199409 | 6 | 118.6 | 0 | 0.04302 | 1.05 |
| A1ZM017348_S_AT | NC0033526 | 8 | 153.1 | NC0199331 | 7 | 173.1 | 0 | 0.03932 | 0.96 |
| A1ZM017348_S_AT | NC0033526 | 8 | 153.1 | NC0199443 | 8 | 72.9 | 0 | −0.03470 | 0.84 |
| A1ZM017348_S_AT | NC0033526 | 8 | 153.1 | NC0199730 | 8 | 79.6 | 0 | −0.03911 | 0.95 |
| A1ZM017348_S_AT | NC0033526 | 8 | 153.1 | NC0199896 | 8 | 96.4 | 0 | 0.03404 | 0.83 |
| A1ZM017348_S_AT | NC0033526 | 8 | 153.1 | NC0035106 | 8 | 151.4 | 0 | 0.05669 | 1.38 |
| A1ZM017348_S_AT | NC0033526 | 8 | 153.1 | NC0033526 | 8 | 153.1 | 1 | −0.06457 | 1.57 |
| A1ZM017348_S_AT | NC0033526 | 8 | 153.1 | NC0014545 | 8 | 155.1 | 0 | −0.05188 | 1.26 |
| A1ZM017348_S_AT | NC0033526 | 8 | 153.1 | NC0199956 | 8 | 169.9 | 0 | 0.04475 | 1.09 |
| A1ZM017348_S_AT | NC0033526 | 8 | 153.1 | NC0056940 | 9 | 0.4 | 0 | 0.03748 | 0.91 |
| A1ZM017348_S_AT | NC0033526 | 8 | 153.1 | NC0157951 | 10 | 48.3 | 0 | 0.03676 | 0.89 |
| A1ZM019225_AT | NC0199896 | 8 | 96.4 | NC0199417 | 1 | 54.3 | 0 | −0.05653 | 0.99 |
| A1ZM019225_AT | NC0199896 | 8 | 96.4 | NC0199318 | 1 | 85.9 | 0 | −0.05985 | 1.05 |
| A1ZM019225_AT | NC0199896 | 8 | 96.4 | NC0157255 | 1 | 87.7 | 0 | −0.04905 | 0.86 |
| A1ZM019225_AT | NC0199896 | 8 | 96.4 | NC0157397 | 1 | 87.7 | 0 | −0.04747 | 0.83 |
| A1ZM019225_AT | NC0199896 | 8 | 96.4 | NC0029053 | 1 | 88.1 | 0 | 0.05760 | 1.01 |
| A1ZM019225_AT | NC0199896 | 8 | 96.4 | NC0199760 | 1 | 179.1 | 0 | −0.05201 | 0.91 |
| A1ZM019225_AT | NC0199896 | 8 | 96.4 | NC0196949 | 2 | 43.4 | 0 | −0.04801 | 0.84 |
| A1ZM019225_AT | NC0199896 | 8 | 96.4 | NC0013275 | 2 | 49.7 | 0 | −0.06374 | 1.12 |
| A1ZM019225_AT | NC0199896 | 8 | 96.4 | NC0170324 | 2 | 94 | 0 | −0.05609 | 0.98 |
| A1ZM019225_AT | NC0199896 | 8 | 96.4 | NC0172302 | 2 | 94 | 0 | −0.05554 | 0.97 |
| A1ZM019225_AT | NC0199896 | 8 | 96.4 | NC0112924 | 3 | 145.3 | 0 | −0.07012 | 1.23 |
| A1ZM019225_AT | NC0199896 | 8 | 96.4 | NC0199794 | 3 | 146 | 0 | −0.05351 | 0.94 |
| A1ZM019225_AT | NC0199896 | 8 | 96.4 | NC0029487 | 4 | 171.1 | 0 | −0.05109 | 0.89 |
| A1ZM019225_AT | NC0199896 | 8 | 96.4 | NC0012281 | 5 | 106.5 | 0 | 0.04832 | 0.85 |
| A1ZM019225_AT | NC0199896 | 8 | 96.4 | NC0200185 | 7 | 147.7 | 0 | −0.05169 | 0.91 |
| A1ZM019225_AT | NC0199896 | 8 | 96.4 | NC0199331 | 7 | 173.1 | 0 | 0.05084 | 0.89 |
| A1ZM019225_AT | NC0199896 | 8 | 96.4 | NC0199443 | 8 | 72.9 | 0 | −0.06256 | 1.10 |
| A1ZM019225_AT | NC0199896 | 8 | 96.4 | NC0194645 | 8 | 76.5 | 0 | −0.05961 | 1.04 |
| A1ZM019225_AT | NC0199896 | 8 | 96.4 | NC0199730 | 8 | 79.6 | 0 | −0.07042 | 1.23 |
| A1ZM019225_AT | NC0199896 | 8 | 96.4 | NC0199735 | 8 | 84.4 | 0 | −0.06934 | 1.21 |
| A1ZM019225_AT | NC0199896 | 8 | 96.4 | NC0199770 | 8 | 96.2 | 0 | −0.05152 | 0.90 |
| A1ZM019225_AT | NC0199896 | 8 | 96.4 | NC0199896 | 8 | 96.4 | 1 | 0.07269 | 1.27 |
| A1ZM019225_AT | NC0199896 | 8 | 96.4 | NC0173626 | 8 | 96.8 | 0 | −0.05668 | 0.99 |
| A1ZM019225_AT | NC0199896 | 8 | 96.4 | NC0111472 | 8 | 103.3 | 0 | 0.04762 | 0.83 |
| A1ZM019225_AT | NC0199896 | 8 | 96.4 | NC0199956 | 8 | 169.9 | 0 | 0.05334 | 0.93 |
| A1ZM020483_A_AT | NC0173951 | 1 | 229.4 | NC0173400 | 1 | 32.2 | 0 | 0.06641 | 1.04 |
| A1ZM020483_A_AT | NC0173951 | 1 | 229.4 | NC0199391 | 1 | 107.5 | 0 | 0.06107 | 0.95 |
| A1ZM020483_A_AT | NC0173951 | 1 | 229.4 | NC0173951 | 1 | 229.4 | 1 | 0.08517 | 1.33 |
| A1ZM020483_A_AT | NC0173951 | 1 | 229.4 | NC0200048 | 1 | 230.2 | 0 | −0.07692 | 1.20 |
| A1ZM020483_A_AT | NC0173951 | 1 | 229.4 | NC0199645 | 3 | 12.1 | 0 | −0.06293 | 0.98 |
| A1ZM020483_A_AT | NC0173951 | 1 | 229.4 | NC0190645 | 3 | 202.4 | 0 | 0.05844 | 0.91 |
| A1ZM020483_A_AT | NC0173951 | 1 | 229.4 | NC0200099 | 5 | 171.1 | 0 | −0.05585 | 0.87 |
| A1ZM020483_A_AT | NC0173951 | 1 | 229.4 | NC0033924 | 6 | 36.2 | 0 | −0.05541 | 0.86 |
| A1ZM020483_A_AT | NC0173951 | 1 | 229.4 | NC0199354 | 7 | 87.4 | 0 | 0.05559 | 0.87 |
| A1ZM020483_A_AT | NC0173951 | 1 | 229.4 | NC0173859 | 7 | 96.8 | 0 | −0.08069 | 1.26 |
| A1ZM020483_A_AT | NC0173951 | 1 | 229.4 | NC0015314 | 7 | 99 | 0 | 0.05563 | 0.87 |
| A1ZM020483_A_AT | NC0173951 | 1 | 229.4 | NC0071001 | 7 | 99.4 | 0 | −0.06199 | 0.97 |
| A1ZM020483_A_AT | NC0173951 | 1 | 229.4 | NC0199889 | 7 | 99.4 | 0 | −0.06058 | 0.95 |
| A1ZM020483_A_AT | NC0173951 | 1 | 229.4 | NC0079306 | 7 | 111.7 | 0 | 0.05855 | 0.91 |
| A1ZM020483_A_AT | NC0173951 | 1 | 229.4 | NC0199879 | 7 | 112.1 | 0 | 0.06074 | 0.95 |
| A1ZM020483_A_AT | NC0173951 | 1 | 229.4 | NC0199800 | 7 | 114.5 | 0 | 0.05811 | 0.91 |
| A1ZM020483_A_AT | NC0173951 | 1 | 229.4 | NC0173358 | 7 | 131 | 0 | −0.05627 | 0.88 |
| A1ZM020483_A_AT | NC0173951 | 1 | 229.4 | NC0200185 | 7 | 147.7 | 0 | −0.07213 | 1.13 |
| A1ZM020483_A_AT | NC0173951 | 1 | 229.4 | NC0000129 | 8 | 16.5 | 0 | −0.06197 | 0.97 |
| A1ZM020483_A_AT | NC0173951 | 1 | 229.4 | NC0199387 | 9 | 33 | 0 | −0.06590 | 1.03 |
| A1ZM021664_AT | NC0200213 | 1 | 67.4 | NC0173332 | 1 | 33.3 | 0 | 0.03849 | 0.95 |
| A1ZM021664_AT | NC0200213 | 1 | 67.4 | NC0199555 | 1 | 34.4 | 0 | −0.03435 | 0.85 |
| A1ZM021664_AT | NC0200213 | 1 | 67.4 | NC0200044 | 1 | 47.5 | 0 | 0.04706 | 1.16 |
| A1ZM021664_AT | NC0200213 | 1 | 67.4 | NC0143873 | 1 | 52.6 | 0 | 0.03757 | 0.92 |
| A1ZM021664_AT | NC0200213 | 1 | 67.4 | NC0199417 | 1 | 54.3 | 0 | −0.03525 | 0.87 |
| A1ZM021664_AT | NC0200213 | 1 | 67.4 | NC0010830 | 1 | 65.8 | 0 | 0.04824 | 1.19 |
| A1ZM021664_AT | NC0200213 | 1 | 67.4 | NC0199971 | 1 | 66.1 | 0 | 0.04693 | 1.15 |
| A1ZM021664_AT | NC0200213 | 1 | 67.4 | NC0158866 | 1 | 66.4 | 0 | −0.03601 | 0.89 |
| A1ZM021664_AT | NC0200213 | 1 | 67.4 | NC0200213 | 1 | 67.4 | 1 | 0.05539 | 1.36 |
| A1ZM021664_AT | NC0200213 | 1 | 67.4 | NC0199911 | 1 | 70.2 | 0 | 0.03720 | 0.92 |
| A1ZM021664_AT | NC0200213 | 1 | 67.4 | NC0043266 | 1 | 71.3 | 0 | −0.03898 | 0.96 |
| A1ZM021664_AT | NC0200213 | 1 | 67.4 | NC0173951 | 1 | 229.4 | 0 | 0.03363 | 0.83 |
| A1ZM021664_AT | NC0200213 | 1 | 67.4 | NC0200138 | 1 | 238.1 | 0 | −0.03686 | 0.91 |
| A1ZM021664_AT | NC0200213 | 1 | 67.4 | NC0199346 | 2 | 50.8 | 0 | −0.03658 | 0.90 |
| A1ZM021664_AT | NC0200213 | 1 | 67.4 | NC0004901 | 2 | 131.6 | 0 | 0.03387 | 0.83 |
| A1ZM021664_AT | NC0200213 | 1 | 67.4 | NC0188475 | 2 | 131.6 | 0 | 0.03748 | 0.92 |
| A1ZM021664_AT | NC0200213 | 1 | 67.4 | NC0193437 | 2 | 131.6 | 0 | 0.03748 | 0.92 |
| A1ZM021664_AT | NC0200213 | 1 | 67.4 | NC0003536 | 3 | 30.9 | 0 | −0.04131 | 1.02 |
| A1ZM021664_AT | NC0200213 | 1 | 67.4 | NC0199913 | 3 | 152.8 | 0 | −0.03855 | 0.95 |
| A1ZM021664_AT | NC0200213 | 1 | 67.4 | NC0174066 | 3 | 168.4 | 0 | 0.03575 | 0.88 |
| A1ZM021664_AT | NC0200213 | 1 | 67.4 | NC0199837 | 4 | 46.8 | 0 | −0.03589 | 0.88 |
| A1ZM021664_AT | NC0200213 | 1 | 67.4 | NC0174148 | 5 | 1.8 | 0 | 0.03515 | 0.86 |
| A1ZM021664_AT | NC0200213 | 1 | 67.4 | NC0036529 | 6 | 43.2 | 0 | 0.05323 | 1.31 |
| A1ZM021664_AT | NC0200213 | 1 | 67.4 | NC0158130 | 6 | 66.3 | 0 | −0.03587 | 0.88 |
| A1ZM021664_AT | NC0200213 | 1 | 67.4 | NC0173859 | 7 | 96.8 | 0 | −0.04351 | 1.07 |
| A1ZM021664_AT | NC0200213 | 1 | 67.4 | NC0199614 | 7 | 108.4 | 0 | −0.05376 | 1.32 |
| A1ZM021664_AT | NC0200213 | 1 | 67.4 | NC0199800 | 7 | 114.5 | 0 | 0.03899 | 0.96 |
| A1ZM021664_AT | NC0200213 | 1 | 67.4 | NC0199355 | 7 | 138.4 | 0 | −0.03508 | 0.86 |
| A1ZM021664_AT | NC0200213 | 1 | 67.4 | NC0200185 | 7 | 147.7 | 0 | −0.05021 | 1.24 |
| A1ZM021664_AT | NC0200213 | 1 | 67.4 | NC0173751 | 7 | 152.4 | 0 | 0.04045 | 1.00 |
| A1ZM021664_AT | NC0200213 | 1 | 67.4 | NC0187978 | 7 | 165.7 | 0 | 0.03661 | 0.90 |
| A1ZM021664_AT | NC0200213 | 1 | 67.4 | NC0199331 | 7 | 173.1 | 0 | 0.04413 | 1.09 |
| A1ZM021664_AT | NC0200213 | 1 | 67.4 | NC0173490 | 8 | 59 | 0 | 0.03519 | 0.87 |
| A1ZM021664_AT | NC0200213 | 1 | 67.4 | NC0199563 | 8 | 115.6 | 0 | 0.04448 | 1.09 |
| A1ZM021664_AT | NC0200213 | 1 | 67.4 | NC0173574 | 9 | 36.6 | 0 | −0.04092 | 1.01 |
| A1ZM021664_AT | NC0200213 | 1 | 67.4 | NC0173530 | 9 | 78.1 | 0 | 0.03837 | 0.94 |
| A1ZM021664_AT | NC0200213 | 1 | 67.4 | NC0014240 | 9 | 103.5 | 0 | −0.04748 | 1.17 |
| A1ZM021664_AT | NC0200213 | 1 | 67.4 | NC0200144 | 9 | 110.4 | 0 | 0.04075 | 1.00 |
| A1ZM021664_AT | NC0200213 | 1 | 67.4 | NC0173478 | 9 | 122.4 | 0 | 0.03760 | 0.92 |
| A1ZM021664_AT | NC0200213 | 1 | 67.4 | NC0173382 | 10 | 27.7 | 0 | 0.04053 | 1.00 |
| A1ZM021664_AT | NC0200213 | 1 | 67.4 | NC0199635 | 10 | 106.4 | 0 | 0.03455 | 0.85 |
| A1ZM023477_S_AT | NC0028658 | 6 | 52.7 | NC0199556 | 1 | 208 | 0 | −0.09500 | 0.90 |
| A1ZM023477_S_AT | NC0028658 | 6 | 52.7 | NC0013275 | 2 | 49.7 | 0 | −0.09499 | 0.90 |
| A1ZM023477_S_AT | NC0028658 | 6 | 52.7 | NC0199346 | 2 | 50.8 | 0 | −0.08919 | 0.85 |
| A1ZM023477_S_AT | NC0028658 | 6 | 52.7 | NC0173731 | 2 | 138.4 | 0 | −0.08730 | 0.83 |
| A1ZM023477_S_AT | NC0028658 | 6 | 52.7 | NC0003536 | 3 | 30.9 | 0 | −0.09965 | 0.95 |
| A1ZM023477_S_AT | NC0028658 | 6 | 52.7 | NC0011194 | 5 | 29.3 | 0 | 0.09735 | 0.93 |
| A1ZM023477_S_AT | NC0028658 | 6 | 52.7 | NC0188018 | 5 | 85.4 | 0 | 0.08641 | 0.82 |
| A1ZM023477_S_AT | NC0028658 | 6 | 52.7 | NC0042917 | 6 | 36.4 | 0 | −0.08706 | 0.83 |
| A1ZM023477_S_AT | NC0028658 | 6 | 52.7 | NC0036529 | 6 | 43.2 | 0 | 0.14186 | 1.35 |
| A1ZM023477_S_AT | NC0028658 | 6 | 52.7 | NC0028658 | 6 | 52.7 | 1 | 0.17865 | 1.70 |
| A1ZM023477_S_AT | NC0028658 | 6 | 52.7 | NC0143950 | 6 | 66 | 0 | 0.09256 | 0.88 |
| A1ZM023477_S_AT | NC0028658 | 6 | 52.7 | NC0199535 | 6 | 75.4 | 0 | 0.09524 | 0.91 |
| A1ZM023477_S_AT | NC0028658 | 6 | 52.7 | NC0199480 | 6 | 99.9 | 0 | 0.09387 | 0.89 |
| A1ZM023477_S_AT | NC0028658 | 6 | 52.7 | NC0200165 | 6 | 99.9 | 0 | 0.11001 | 1.05 |
| A1ZM023477_S_AT | NC0028658 | 6 | 52.7 | NC0173876 | 6 | 104.2 | 0 | 0.09092 | 0.87 |
| A1ZM023477_S_AT | NC0028658 | 6 | 52.7 | NC0195223 | 6 | 118.6 | 0 | −0.09705 | 0.92 |
| A1ZM025661_AT | NC0173436 | 3 | 86 | NC0200044 | 1 | 47.5 | 0 | 0.09124 | 1.01 |
| A1ZM025661_AT | NC0173436 | 3 | 86 | NC0170324 | 2 | 94 | 0 | −0.07836 | 0.87 |
| A1ZM025661_AT | NC0173436 | 3 | 86 | NC0172302 | 2 | 94 | 0 | −0.07992 | 0.88 |
| A1ZM025661_AT | NC0173436 | 3 | 86 | NC0109315 | 3 | 77.4 | 0 | −0.07482 | 0.83 |
| A1ZM025661_AT | NC0173436 | 3 | 86 | NC0199803 | 3 | 85.6 | 0 | −0.08863 | 0.98 |
| A1ZM025661_AT | NC0173436 | 3 | 86 | NC0173436 | 3 | 86 | 1 | −0.13897 | 1.53 |
| A1ZM025661_AT | NC0173436 | 3 | 86 | NC0199763 | 3 | 86 | 0 | 0.08728 | 0.96 |
| A1ZM025661_AT | NC0173436 | 3 | 86 | NC0199947 | 3 | 86.4 | 0 | 0.10339 | 1.14 |
| A1ZM025661_AT | NC0173436 | 3 | 86 | NC0199372 | 3 | 87.1 | 0 | 0.08128 | 0.90 |
| A1ZM025661_AT | NC0173436 | 3 | 86 | NC0199589 | 3 | 91.5 | 0 | 0.10045 | 1.11 |
| A1ZM025661_AT | NC0173436 | 3 | 86 | NC0199676 | 3 | 91.5 | 0 | 0.11469 | 1.27 |
| A1ZM025661_AT | NC0173436 | 3 | 86 | NC0077263 | 4 | 50.6 | 0 | −0.07301 | 0.81 |
| A1ZM025661_AT | NC0173436 | 3 | 86 | NC0200212 | 5 | 176.4 | 0 | 0.07555 | 0.83 |
| A1ZM025661_AT | NC0173436 | 3 | 86 | NC0199915 | 6 | 84.9 | 0 | −0.08163 | 0.90 |
| A1ZM025661_AT | NC0173436 | 3 | 86 | NC0187978 | 7 | 165.7 | 0 | 0.07929 | 0.88 |
| A1ZM025661_AT | NC0173436 | 3 | 86 | NC0199730 | 8 | 79.6 | 0 | −0.07372 | 0.81 |
| A1ZM025769_AT | NC0199346 | 2 | 50.8 | NC0013275 | 2 | 49.7 | 0 | 0.06653 | 1.01 |
| A1ZM025769_AT | NC0199346 | 2 | 50.8 | NC0199346 | 2 | 50.8 | 1 | 0.09069 | 1.37 |
| A1ZM025769_AT | NC0199346 | 2 | 50.8 | NC0002630 | 2 | 54.1 | 0 | −0.06485 | 0.98 |
| A1ZM025769_AT | NC0199346 | 2 | 50.8 | NC0199524 | 2 | 141.5 | 0 | −0.06229 | 0.94 |
| A1ZM025769_AT | NC0199346 | 2 | 50.8 | NC0030963 | 3 | 87 | 0 | −0.06183 | 0.94 |
| A1ZM025769_AT | NC0199346 | 2 | 50.8 | NC0200081 | 3 | 115.9 | 0 | 0.06965 | 1.06 |
| A1ZM025769_AT | NC0199346 | 2 | 50.8 | NC0199794 | 3 | 146 | 0 | 0.06877 | 1.04 |
| A1ZM025769_AT | NC0199346 | 2 | 50.8 | NC0200120 | 5 | 75.4 | 0 | −0.08028 | 1.22 |
| A1ZM025769_AT | NC0199346 | 2 | 50.8 | NC0199447 | 5 | 80.6 | 0 | 0.06054 | 0.92 |
| A1ZM025769_AT | NC0199346 | 2 | 50.8 | NC0188018 | 5 | 85.4 | 0 | −0.05742 | 0.87 |
| A1ZM025769_AT | NC0199346 | 2 | 50.8 | NC0078090 | 5 | 141.8 | 0 | 0.05658 | 0.86 |
| A1ZM025769_AT | NC0199346 | 2 | 50.8 | NC0173396 | 5 | 143.1 | 0 | 0.07140 | 1.08 |
| A1ZM025769_AT | NC0199346 | 2 | 50.8 | NC0042059 | 5 | 143.2 | 0 | 0.07263 | 1.10 |
| A1ZM025769_AT | NC0199346 | 2 | 50.8 | NC0173843 | 5 | 143.2 | 0 | −0.05970 | 0.90 |
| A1ZM025769_AT | NC0199346 | 2 | 50.8 | NC0033924 | 6 | 36.2 | 0 | 0.06348 | 0.96 |
| A1ZM025769_AT | NC0199346 | 2 | 50.8 | NC0042917 | 6 | 36.4 | 0 | 0.05622 | 0.85 |
| A1ZM025769_AT | NC0199346 | 2 | 50.8 | NC0195223 | 6 | 118.6 | 0 | 0.05601 | 0.85 |
| A1ZM025769_AT | NC0199346 | 2 | 50.8 | NC0173794 | 6 | 131.1 | 0 | 0.06649 | 1.01 |
| A1ZM025769_AT | NC0199346 | 2 | 50.8 | NC0199685 | 7 | 28.6 | 0 | 0.06401 | 0.97 |
| A1ZM025769_AT | NC0199346 | 2 | 50.8 | NC0199331 | 7 | 173.1 | 0 | −0.05964 | 0.90 |
| A1ZM025982_AT | NC0199730 | 8 | 79.6 | NC0194645 | 8 | 76.5 | 0 | 0.13598 | 0.94 |
| A1ZM025982_AT | NC0199730 | 8 | 79.6 | NC0199730 | 8 | 79.6 | 1 | 0.17787 | 1.23 |
| A1ZM025982_AT | NC0199730 | 8 | 79.6 | NC0199735 | 8 | 84.4 | 0 | 0.12829 | 0.88 |
| A1ZM025982_AT | NC0199730 | 8 | 79.6 | NC0199770 | 8 | 96.2 | 0 | 0.14535 | 1.00 |
| A1ZM025982_AT | NC0199730 | 8 | 79.6 | NC0199896 | 8 | 96.4 | 0 | −0.14701 | 1.01 |
| A1ZM025982_AT | NC0199730 | 8 | 79.6 | NC0173626 | 8 | 96.8 | 0 | 0.12956 | 0.89 |
| A1ZM026053_AT | NC0199722 | 9 | 79.6 | NC0036726 | 1 | 35.2 | 0 | 0.03990 | 0.80 |
| A1ZM026053_AT | NC0199722 | 9 | 79.6 | NC0196949 | 2 | 43.4 | 0 | 0.05657 | 1.14 |
| A1ZM026053_AT | NC0199722 | 9 | 79.6 | NC0013275 | 2 | 49.7 | 0 | 0.04975 | 1.00 |
| A1ZM026053_AT | NC0199722 | 9 | 79.6 | NC0199741 | 3 | 125.7 | 0 | 0.04102 | 0.83 |
| A1ZM026053_AT | NC0199722 | 9 | 79.6 | NC0015965 | 3 | 140.8 | 0 | 0.05190 | 1.05 |
| A1ZM026053_AT | NC0199722 | 9 | 79.6 | NC0199921 | 3 | 141.1 | 0 | −0.04084 | 0.82 |
| A1ZM026053_AT | NC0199722 | 9 | 79.6 | NC0199794 | 3 | 146 | 0 | 0.04055 | 0.82 |
| A1ZM026053_AT | NC0199722 | 9 | 79.6 | NC0190032 | 3 | 147.8 | 0 | 0.05220 | 1.05 |
| A1ZM026053_AT | NC0199722 | 9 | 79.6 | NC0175374 | 4 | 21.6 | 0 | 0.04212 | 0.85 |
| A1ZM026053_AT | NC0199722 | 9 | 79.6 | NC0199978 | 4 | 70.6 | 0 | 0.03976 | 0.80 |
| A1ZM026053_AT | NC0199722 | 9 | 79.6 | NC0199376 | 5 | 54.4 | 0 | −0.04168 | 0.84 |
| A1ZM026053_AT | NC0199722 | 9 | 79.6 | NC0200112 | 6 | 105.6 | 0 | −0.04211 | 0.85 |
| A1ZM026053_AT | NC0199722 | 9 | 79.6 | NC0199723 | 7 | 40.4 | 0 | −0.05134 | 1.03 |
| A1ZM026053_AT | NC0199722 | 9 | 79.6 | NC0173476 | 7 | 44.2 | 0 | 0.04420 | 0.89 |
| A1ZM026053_AT | NC0199722 | 9 | 79.6 | NC0158373 | 8 | 148.9 | 0 | −0.04189 | 0.84 |
| A1ZM026053_AT | NC0199722 | 9 | 79.6 | NC0199880 | 8 | 161.8 | 0 | 0.04418 | 0.89 |
| A1ZM026053_AT | NC0199722 | 9 | 79.6 | NC0029744 | 9 | 67.4 | 0 | −0.06562 | 1.32 |
| A1ZM026053_AT | NC0199722 | 9 | 79.6 | NC0173953 | 9 | 67.4 | 0 | 0.05924 | 1.19 |
| A1ZM026053_AT | NC0199722 | 9 | 79.6 | NC0020872 | 9 | 76.3 | 0 | 0.05112 | 1.03 |
| A1ZM026053_AT | NC0199722 | 9 | 79.6 | NC0173530 | 9 | 78.1 | 0 | −0.05188 | 1.05 |
| A1ZM026053_AT | NC0199722 | 9 | 79.6 | NC0199722 | 9 | 79.6 | 1 | −0.06739 | 1.36 |
| A1ZM026053_AT | NC0199722 | 9 | 79.6 | NC0153579 | 9 | 97.3 | 0 | 0.04037 | 0.81 |
| A1ZM026053_AT | NC0199722 | 9 | 79.6 | NC0014240 | 9 | 103.5 | 0 | 0.04798 | 0.97 |
| A1ZM026053_AT | NC0199722 | 9 | 79.6 | NC0030512 | 9 | 103.6 | 0 | 0.06668 | 1.34 |
| A1ZM026053_AT | NC0199722 | 9 | 79.6 | NC0200144 | 9 | 110.4 | 0 | −0.06515 | 1.31 |
| A1ZM026053_AT | NC0199722 | 9 | 79.6 | NC0051922 | 9 | 110.5 | 0 | −0.04482 | 0.90 |
| A1ZM026053_AT | NC0199722 | 9 | 79.6 | NC0029024 | 9 | 116.9 | 0 | −0.04286 | 0.86 |
| A1ZM026053_AT | NC0199722 | 9 | 79.6 | NC0199898 | 9 | 119.2 | 0 | −0.04667 | 0.94 |
| A1ZM026053_AT | NC0199722 | 9 | 79.6 | NC0173478 | 9 | 122.4 | 0 | −0.05517 | 1.11 |
| A1ZM026053_AT | NC0199722 | 9 | 79.6 | NC0174164 | 9 | 147.9 | 0 | 0.04397 | 0.89 |
| A1ZM026053_AT | NC0199722 | 9 | 79.6 | NC0011002 | 10 | 66.5 | 0 | 0.04723 | 0.95 |
| A1ZM026886_AT | NC0038733 | 7 | 106.5 | NC0200147 | 5 | 139.5 | 0 | −0.15008 | 0.98 |
| A1ZM026886_AT | NC0038733 | 7 | 106.5 | NC0038733 | 7 | 106.5 | 1 | 0.15955 | 1.04 |
| A1ZM026886_AT | NC0038733 | 7 | 106.5 | NC0200123 | 7 | 107.2 | 0 | 0.15847 | 1.03 |
| A1ZM026886_AT | NC0038733 | 7 | 106.5 | NC0173530 | 9 | 78.1 | 0 | 0.14797 | 0.96 |
| A1ZM026886_AT | NC0038733 | 7 | 106.5 | NC0199722 | 9 | 79.6 | 0 | 0.15101 | 0.98 |
| A1ZM030937_AT | NC0173436 | 3 | 86 | NC0032745 | 1 | 94.5 | 0 | −0.04906 | 0.90 |
| A1ZM030937_AT | NC0173436 | 3 | 86 | NC0173334 | 1 | 102.8 | 0 | 0.04836 | 0.89 |
| A1ZM030937_AT | NC0173436 | 3 | 86 | NC0173484 | 1 | 103.2 | 0 | 0.05206 | 0.95 |
| A1ZM030937_AT | NC0173436 | 3 | 86 | NC0199346 | 2 | 50.8 | 0 | −0.04534 | 0.83 |
| A1ZM030937_AT | NC0173436 | 3 | 86 | NC0195038 | 3 | 74.2 | 0 | −0.05017 | 0.92 |
| A1ZM030937_AT | NC0173436 | 3 | 86 | NC0109315 | 3 | 77.4 | 0 | −0.06267 | 1.15 |
| A1ZM030937_AT | NC0173436 | 3 | 86 | NC0173641 | 3 | 80.9 | 0 | 0.06448 | 1.18 |
| A1ZM030937_AT | NC0173436 | 3 | 86 | NC0199803 | 3 | 85.6 | 0 | −0.04631 | 0.85 |
| A1ZM030937_AT | NC0173436 | 3 | 86 | NC0173436 | 3 | 86 | 1 | −0.08150 | 1.49 |
| A1ZM030937_AT | NC0173436 | 3 | 86 | NC0173845 | 3 | 87 | 0 | −0.04699 | 0.86 |
| A1ZM030937_AT | NC0173436 | 3 | 86 | NC0199589 | 3 | 91.5 | 0 | 0.07812 | 1.43 |
| A1ZM030937_AT | NC0173436 | 3 | 86 | NC0199676 | 3 | 91.5 | 0 | 0.06862 | 1.26 |
| A1ZM030937_AT | NC0173436 | 3 | 86 | NC0200156 | 3 | 96.1 | 0 | 0.04822 | 0.88 |
| A1ZM030937_AT | NC0173436 | 3 | 86 | NC0015965 | 3 | 140.8 | 0 | −0.05089 | 0.93 |
| A1ZM030937_AT | NC0173436 | 3 | 86 | NC0199794 | 3 | 146 | 0 | −0.05571 | 1.02 |
| A1ZM030937_AT | NC0173436 | 3 | 86 | NC0199814 | 3 | 208.5 | 0 | −0.04626 | 0.85 |
| A1ZM030937_AT | NC0173436 | 3 | 86 | NC0200132 | 4 | 128.7 | 0 | −0.04826 | 0.88 |
| A1ZM030937_AT | NC0173436 | 3 | 86 | NC0192676 | 5 | 88.2 | 0 | 0.04633 | 0.85 |
| A1ZM030937_AT | NC0173436 | 3 | 86 | NC0199805 | 8 | 51.7 | 0 | −0.04464 | 0.82 |
| A1ZM030937_AT | NC0173436 | 3 | 86 | NC0199387 | 9 | 33 | 0 | −0.04520 | 0.83 |
| A1ZM030937_AT | NC0173436 | 3 | 86 | NC0029744 | 9 | 67.4 | 0 | 0.04860 | 0.89 |
| A1ZM030937_AT | NC0173436 | 3 | 86 | NC0173953 | 9 | 67.4 | 0 | −0.04649 | 0.85 |
| A1ZM030937_AT | NC0173436 | 3 | 86 | NC0020872 | 9 | 76.3 | 0 | −0.05111 | 0.94 |
| A1ZM030937_AT | NC0173436 | 3 | 86 | NC0173530 | 9 | 78.1 | 0 | 0.05879 | 1.08 |
| A1ZM030937_AT | NC0173436 | 3 | 86 | NC0199722 | 9 | 79.6 | 0 | 0.05439 | 1.00 |
| A1ZM030937_AT | NC0173436 | 3 | 86 | NC0014240 | 9 | 103.5 | 0 | −0.06198 | 1.13 |
| A1ZM030937_AT | NC0173436 | 3 | 86 | NC0030512 | 9 | 103.6 | 0 | −0.05445 | 1.00 |
| A1ZM030937_AT | NC0173436 | 3 | 86 | NC0200144 | 9 | 110.4 | 0 | 0.06019 | 1.10 |
| A1ZM030937_AT | NC0173436 | 3 | 86 | NC0157951 | 10 | 48.3 | 0 | 0.04383 | 0.80 |
| A1ZM031333_AT | NC0199722 | 9 | 79.6 | NC0013275 | 2 | 49.7 | 0 | −0.08936 | 0.94 |
| A1ZM031333_AT | NC0199722 | 9 | 79.6 | NC0199346 | 2 | 50.8 | 0 | −0.08901 | 0.94 |
| A1ZM031333_AT | NC0199722 | 9 | 79.6 | NC0009470 | 3 | 88 | 0 | 0.08570 | 0.90 |
| A1ZM031333_AT | NC0199722 | 9 | 79.6 | NC0015965 | 3 | 140.8 | 0 | −0.09789 | 1.03 |
| A1ZM031333_AT | NC0199722 | 9 | 79.6 | NC0199794 | 3 | 146 | 0 | −0.08035 | 0.85 |
| A1ZM031333_AT | NC0199722 | 9 | 79.6 | NC0199913 | 3 | 152.8 | 0 | −0.07651 | 0.81 |
| A1ZM031333_AT | NC0199722 | 9 | 79.6 | NC0173476 | 7 | 44.2 | 0 | −0.08394 | 0.89 |
| A1ZM031333_AT | NC0199722 | 9 | 79.6 | NC0200185 | 7 | 147.7 | 0 | −0.07899 | 0.83 |
| A1ZM031333_AT | NC0199722 | 9 | 79.6 | NC0020872 | 9 | 76.3 | 0 | −0.10514 | 1.11 |
| A1ZM031333_AT | NC0199722 | 9 | 79.6 | NC0173530 | 9 | 78.1 | 0 | 0.11429 | 1.21 |
| A1ZM031333_AT | NC0199722 | 9 | 79.6 | NC0199722 | 9 | 79.6 | 1 | 0.13037 | 1.38 |
| A1ZM031333_AT | NC0199722 | 9 | 79.6 | NC0011002 | 10 | 66.5 | 0 | −0.09068 | 0.96 |
| A1ZM032249_AT | NC0173530 | 9 | 78.1 | NC0173951 | 1 | 229.4 | 0 | 0.18168 | 0.92 |
| A1ZM032249_AT | NC0173530 | 9 | 78.1 | NC0199346 | 2 | 50.8 | 0 | −0.07136 | 0.82 |
| A1ZM032249_AT | NC0173530 | 9 | 78.1 | NC0199669 | 3 | 122.8 | 0 | 0.22196 | 1.09 |
| A1ZM032249_AT | NC0173530 | 9 | 78.1 | NC0015965 | 3 | 140.8 | 0 | −0.06541 | 1.03 |
| A1ZM032249_AT | NC0173530 | 9 | 78.1 | NC0199794 | 3 | 146 | 0 | −0.08412 | 0.96 |
| A1ZM032249_AT | NC0173530 | 9 | 78.1 | NC0190032 | 3 | 147.8 | 0 | −0.04389 | 0.83 |
| A1ZM032249_AT | NC0173530 | 9 | 78.1 | NC0173476 | 7 | 44.2 | 0 | −0.15596 | 0.84 |
| A1ZM032249_AT | NC0173530 | 9 | 78.1 | NC0173435 | 7 | 71.6 | 0 | −0.28189 | 1.26 |
| A1ZM032249_AT | NC0173530 | 9 | 78.1 | NC0199355 | 7 | 138.4 | 0 | −0.25414 | 1.21 |
| A1ZM032249_AT | NC0173530 | 9 | 78.1 | NC0020872 | 9 | 76.3 | 0 | 0.01853 | 0.92 |
| A1ZM032249_AT | NC0173530 | 9 | 78.1 | NC0173530 | 9 | 78.1 | 1 | 0.08986 | 1.05 |
| A1ZM032249_AT | NC0173530 | 9 | 78.1 | NC0199722 | 9 | 79.6 | 0 | 0.03780 | 0.95 |
| A1ZM033184_AT | NC0200093 | 5 | 131.8 | NC0009148 | 1 | 213 | 0 | 0.08631 | 1.00 |
| A1ZM033184_AT | NC0200093 | 5 | 131.8 | NC0013275 | 2 | 49.7 | 0 | −0.08063 | 0.93 |
| A1ZM033184_AT | NC0200093 | 5 | 131.8 | NC0199346 | 2 | 50.8 | 0 | −0.08481 | 0.98 |
| A1ZM033184_AT | NC0200093 | 5 | 131.8 | NC0174062 | 2 | 129.5 | 0 | −0.07905 | 0.92 |
| A1ZM033184_AT | NC0200093 | 5 | 131.8 | NC0003536 | 3 | 30.9 | 0 | −0.08241 | 0.96 |
| A1ZM033184_AT | NC0200093 | 5 | 131.8 | NC0015965 | 3 | 140.8 | 0 | −0.09366 | 1.09 |
| A1ZM033184_AT | NC0200093 | 5 | 131.8 | NC0035568 | 3 | 166.8 | 0 | 0.08196 | 0.95 |
| A1ZM033184_AT | NC0200093 | 5 | 131.8 | NC0200093 | 5 | 131.8 | 1 | −0.09789 | 1.13 |
| A1ZM033184_AT | NC0200093 | 5 | 131.8 | NC0200165 | 6 | 99.9 | 0 | 0.08093 | 0.94 |
| A1ZM033184_AT | NC0200093 | 5 | 131.8 | NC0200185 | 7 | 147.7 | 0 | −0.09041 | 1.05 |
| A1ZM033184_AT | NC0200093 | 5 | 131.8 | NC0199563 | 8 | 115.6 | 0 | 0.08328 | 0.97 |
| A1ZM033184_AT | NC0200093 | 5 | 131.8 | NC0173530 | 9 | 78.1 | 0 | 0.08423 | 0.98 |
| A1ZM033184_AT | NC0200093 | 5 | 131.8 | NC0199722 | 9 | 79.6 | 0 | 0.08384 | 0.97 |
| A1ZM033184_AT | NC0200093 | 5 | 131.8 | NC0014240 | 9 | 103.5 | 0 | −0.08870 | 1.03 |
| A1ZM033184_AT | NC0200093 | 5 | 131.8 | NC0011002 | 10 | 66.5 | 0 | −0.09359 | 1.08 |
| A1ZM033611_AT | NC0014240 | 9 | 103.5 | NC0199555 | 1 | 34.4 | 0 | −0.07035 | 0.95 |
| A1ZM033611_AT | NC0014240 | 9 | 103.5 | NC0043266 | 1 | 71.3 | 0 | −0.06642 | 0.90 |
| A1ZM033611_AT | NC0014240 | 9 | 103.5 | NC0199346 | 2 | 50.8 | 0 | −0.07477 | 1.01 |
| A1ZM033611_AT | NC0014240 | 9 | 103.5 | NC0002630 | 2 | 54.1 | 0 | 0.07037 | 0.95 |
| A1ZM033611_AT | NC0014240 | 9 | 103.5 | NC0109315 | 3 | 77.4 | 0 | −0.07120 | 0.96 |
| A1ZM033611_AT | NC0014240 | 9 | 103.5 | NC0112924 | 3 | 145.3 | 0 | −0.07228 | 0.98 |
| A1ZM033611_AT | NC0014240 | 9 | 103.5 | NC0199388 | 7 | 62.8 | 0 | −0.06716 | 0.91 |
| A1ZM033611_AT | NC0014240 | 9 | 103.5 | NC0200064 | 7 | 65.3 | 0 | 0.06625 | 0.90 |
| A1ZM033611_AT | NC0014240 | 9 | 103.5 | NC0199834 | 8 | 84 | 0 | −0.07528 | 1.02 |
| A1ZM033611_AT | NC0014240 | 9 | 103.5 | NC0199581 | 8 | 91.6 | 0 | 0.07413 | 1.00 |
| A1ZM033611_AT | NC0014240 | 9 | 103.5 | NC0173350 | 8 | 104 | 0 | 0.07203 | 0.98 |
| A1ZM033611_AT | NC0014240 | 9 | 103.5 | NC0199563 | 8 | 115.6 | 0 | 0.07883 | 1.07 |
| A1ZM033611_AT | NC0014240 | 9 | 103.5 | NC0029744 | 9 | 67.4 | 0 | 0.07238 | 0.98 |
| A1ZM033611_AT | NC0014240 | 9 | 103.5 | NC0173953 | 9 | 67.4 | 0 | −0.07353 | 1.00 |
| A1ZM033611_AT | NC0014240 | 9 | 103.5 | NC0173530 | 9 | 78.1 | 0 | 0.07233 | 0.98 |
| A1ZM033611_AT | NC0014240 | 9 | 103.5 | NC0199722 | 9 | 79.6 | 0 | 0.07202 | 0.98 |
| A1ZM033611_AT | NC0014240 | 9 | 103.5 | NC0014240 | 9 | 103.5 | 1 | −0.09958 | 1.35 |
| A1ZM033611_AT | NC0014240 | 9 | 103.5 | NC0030512 | 9 | 103.6 | 0 | −0.06705 | 0.91 |
| A1ZM033611_AT | NC0014240 | 9 | 103.5 | NC0199810 | 10 | 48.3 | 0 | 0.07938 | 1.08 |
| A1ZM033956_AT | NC0174062 | 2 | 129.5 | NC0199661 | 1 | 202.9 | 0 | −0.15110 | 0.99 |
| A1ZM033956_AT | NC0174062 | 2 | 129.5 | NC0174062 | 2 | 129.5 | 1 | −0.16092 | 1.06 |
| A1ZM033956_AT | NC0174062 | 2 | 129.5 | NC0174066 | 3 | 168.4 | 0 | 0.14772 | 0.97 |
| A1ZM033956_AT | NC0174062 | 2 | 129.5 | NC0195223 | 6 | 118.6 | 0 | −0.15374 | 1.01 |
| A1ZM033956_AT | NC0174062 | 2 | 129.5 | NC0199958 | 6 | 142 | 0 | −0.15727 | 1.03 |
| A1ZM033956_AT | NC0174062 | 2 | 129.5 | NC0020611 | 7 | 10 | 0 | 0.15420 | 1.01 |
| A1ZM033956_AT | NC0174062 | 2 | 129.5 | NC0174551 | 7 | 11.2 | 0 | −0.14694 | 0.97 |
| A1ZM033956_AT | NC0174062 | 2 | 129.5 | NC0038733 | 7 | 106.5 | 0 | 0.14517 | 0.95 |
| A1ZM039148_AT | NC0003536 | 3 | 30.9 | NC0003536 | 3 | 30.9 | 1 | −0.13051 | 1.13 |
| A1ZM039148_AT | NC0003536 | 3 | 30.9 | NC0015965 | 3 | 140.8 | 0 | −0.12034 | 1.04 |
| A1ZM039148_AT | NC0003536 | 3 | 30.9 | NC0190032 | 3 | 147.8 | 0 | −0.12714 | 1.10 |
| A1ZM039148_AT | NC0003536 | 3 | 30.9 | NC0199913 | 3 | 152.8 | 0 | −0.11006 | 0.96 |
| A1ZM039148_AT | NC0003536 | 3 | 30.9 | NC0199485 | 3 | 175.1 | 0 | 0.10710 | 0.93 |
| A1ZM039148_AT | NC0003536 | 3 | 30.9 | NC0200093 | 5 | 131.8 | 0 | −0.11526 | 1.00 |
| A1ZM039148_AT | NC0003536 | 3 | 30.9 | NC0173584 | 7 | 8.5 | 0 | 0.10646 | 0.92 |
| A1ZM039148_AT | NC0003536 | 3 | 30.9 | NC0174551 | 7 | 11.2 | 0 | −0.10701 | 0.93 |
| A1ZM039148_AT | NC0003536 | 3 | 30.9 | NC0173476 | 7 | 44.2 | 0 | −0.10752 | 0.93 |
| A1ZM039148_AT | NC0003536 | 3 | 30.9 | NC0199805 | 8 | 51.7 | 0 | −0.11554 | 1.00 |
| A1ZM039148_AT | NC0003536 | 3 | 30.9 | NC0199563 | 8 | 115.6 | 0 | 0.11762 | 1.02 |
| A1ZM043521_AT | NC0199635 | 10 | 106.4 | NC0173400 | 1 | 32.2 | 0 | 0.05198 | 0.88 |
| A1ZM043521_AT | NC0199635 | 10 | 106.4 | NC0200044 | 1 | 47.5 | 0 | 0.05409 | 0.91 |
| A1ZM043521_AT | NC0199635 | 10 | 106.4 | NC0143873 | 1 | 52.6 | 0 | 0.05787 | 0.98 |
| A1ZM043521_AT | NC0199635 | 10 | 106.4 | NC0000399 | 3 | 121.1 | 0 | −0.05194 | 0.88 |
| A1ZM043521_AT | NC0199635 | 10 | 106.4 | NC0199741 | 3 | 125.7 | 0 | −0.06620 | 1.12 |
| A1ZM043521_AT | NC0199635 | 10 | 106.4 | NC0015965 | 3 | 140.8 | 0 | −0.05091 | 0.86 |
| A1ZM043521_AT | NC0199635 | 10 | 106.4 | NC0112924 | 3 | 145.3 | 0 | −0.06724 | 1.13 |
| A1ZM043521_AT | NC0199635 | 10 | 106.4 | NC0199913 | 3 | 152.8 | 0 | −0.05637 | 0.95 |
| A1ZM043521_AT | NC0199635 | 10 | 106.4 | NC0199877 | 4 | 127.9 | 0 | −0.05026 | 0.85 |
| A1ZM043521_AT | NC0199635 | 10 | 106.4 | NC0024368 | 4 | 162.6 | 0 | −0.05703 | 0.96 |
| A1ZM043521_AT | NC0199635 | 10 | 106.4 | NC0194397 | 6 | 38.4 | 0 | −0.06619 | 1.12 |
| A1ZM043521_AT | NC0199635 | 10 | 106.4 | NC0199422 | 6 | 41.1 | 0 | 0.07342 | 1.24 |
| A1ZM043521_AT | NC0199635 | 10 | 106.4 | NC0158250 | 7 | 24.3 | 0 | 0.07278 | 1.23 |
| A1ZM043521_AT | NC0199635 | 10 | 106.4 | NC0199388 | 7 | 62.8 | 0 | −0.05027 | 0.85 |
| A1ZM043521_AT | NC0199635 | 10 | 106.4 | NC0173859 | 7 | 96.8 | 0 | −0.06135 | 1.03 |
| A1ZM043521_AT | NC0199635 | 10 | 106.4 | NC0199355 | 7 | 138.4 | 0 | −0.06030 | 1.02 |
| A1ZM043521_AT | NC0199635 | 10 | 106.4 | NC0200185 | 7 | 147.7 | 0 | −0.06104 | 1.03 |
| A1ZM043521_AT | NC0199635 | 10 | 106.4 | NC0000129 | 8 | 16.5 | 0 | −0.06128 | 1.03 |
| A1ZM043521_AT | NC0199635 | 10 | 106.4 | NC0158373 | 8 | 148.9 | 0 | 0.05163 | 0.87 |
| A1ZM043521_AT | NC0199635 | 10 | 106.4 | NC0173530 | 9 | 78.1 | 0 | 0.04991 | 0.84 |
| A1ZM043521_AT | NC0199635 | 10 | 106.4 | NC0199722 | 9 | 79.6 | 0 | 0.04988 | 0.84 |
| A1ZM043521_AT | NC0199635 | 10 | 106.4 | NC0010789 | 9 | 96.9 | 0 | 0.06449 | 1.09 |
| A1ZM043521_AT | NC0199635 | 10 | 106.4 | NC0014240 | 9 | 103.5 | 0 | −0.05926 | 1.00 |
| A1ZM043521_AT | NC0199635 | 10 | 106.4 | NC0173382 | 10 | 27.7 | 0 | 0.06290 | 1.06 |
| A1ZM043521_AT | NC0199635 | 10 | 106.4 | NC0199810 | 10 | 48.3 | 0 | 0.05341 | 0.90 |
| A1ZM043521_AT | NC0199635 | 10 | 106.4 | NC0173834 | 10 | 49.1 | 0 | −0.05819 | 0.98 |
| A1ZM043521_AT | NC0199635 | 10 | 106.4 | NC0197151 | 10 | 92.4 | 0 | 0.05031 | 0.85 |
| A1ZM043521_AT | NC0199635 | 10 | 106.4 | NC0199635 | 10 | 106.4 | 1 | 0.07644 | 1.29 |
| A1ZM043652_S_AT | NC0173626 | 8 | 96.8 | NC0199461 | 1 | 176.7 | 0 | 0.06976 | 0.95 |
| A1ZM043652_S_AT | NC0173626 | 8 | 96.8 | NC0013275 | 2 | 49.7 | 0 | 0.06169 | 0.84 |
| A1ZM043652_S_AT | NC0173626 | 8 | 96.8 | NC0199465 | 3 | 81.6 | 0 | −0.07271 | 1.00 |
| A1ZM043652_S_AT | NC0173626 | 8 | 96.8 | NC0199853 | 3 | 81.6 | 0 | 0.06779 | 0.93 |
| A1ZM043652_S_AT | NC0173626 | 8 | 96.8 | NC0199803 | 3 | 85.6 | 0 | 0.07606 | 1.04 |
| A1ZM043652_S_AT | NC0173626 | 8 | 96.8 | NC0173436 | 3 | 86 | 0 | 0.06687 | 0.92 |
| A1ZM043652_S_AT | NC0173626 | 8 | 96.8 | NC0198241 | 3 | 102 | 0 | 0.06169 | 0.84 |
| A1ZM043652_S_AT | NC0173626 | 8 | 96.8 | NC0199826 | 4 | 190.8 | 0 | 0.06760 | 0.93 |
| A1ZM043652_S_AT | NC0173626 | 8 | 96.8 | NC0199614 | 7 | 108.4 | 0 | 0.06164 | 0.84 |
| A1ZM043652_S_AT | NC0173626 | 8 | 96.8 | NC0199730 | 8 | 79.6 | 0 | 0.06397 | 0.88 |
| A1ZM043652_S_AT | NC0173626 | 8 | 96.8 | NC0199735 | 8 | 84.4 | 0 | 0.07629 | 1.04 |
| A1ZM043652_S_AT | NC0173626 | 8 | 96.8 | NC0199770 | 8 | 96.2 | 0 | 0.08659 | 1.19 |
| A1ZM043652_S_AT | NC0173626 | 8 | 96.8 | NC0199896 | 8 | 96.4 | 0 | −0.06912 | 0.95 |
| A1ZM043652_S_AT | NC0173626 | 8 | 96.8 | NC0173626 | 8 | 96.8 | 1 | 0.09984 | 1.37 |
| A1ZM043652_S_AT | NC0173626 | 8 | 96.8 | NC0111472 | 8 | 103.3 | 0 | −0.08930 | 1.22 |
| A1ZM043652_S_AT | NC0173626 | 8 | 96.8 | NC0107256 | 9 | 68.5 | 0 | −0.06532 | 0.89 |
| A1ZM044683_X_AT | NC0174062 | 2 | 129.5 | NC0010830 | 1 | 65.8 | 0 | −0.10186 | 1.09 |
| A1ZM044683_X_AT | NC0174062 | 2 | 129.5 | NC0158866 | 1 | 66.4 | 0 | 0.07810 | 0.84 |
| A1ZM044683_X_AT | NC0174062 | 2 | 129.5 | NC0199911 | 1 | 70.2 | 0 | −0.08326 | 0.89 |
| A1ZM044683_X_AT | NC0174062 | 2 | 129.5 | NC0173453 | 1 | 172.2 | 0 | −0.08166 | 0.88 |
| A1ZM044683_X_AT | NC0174062 | 2 | 129.5 | NC0015937 | 1 | 197.7 | 0 | −0.09149 | 0.98 |
| A1ZM044683_X_AT | NC0174062 | 2 | 129.5 | NC0199310 | 2 | 75.7 | 0 | 0.07554 | 0.81 |
| A1ZM044683_X_AT | NC0174062 | 2 | 129.5 | NC0174061 | 2 | 118.1 | 0 | 0.10556 | 1.13 |
| A1ZM044683_X_AT | NC0174062 | 2 | 129.5 | NC0200164 | 2 | 120.4 | 0 | −0.11633 | 1.25 |
| A1ZM044683_X_AT | NC0174062 | 2 | 129.5 | NC0174062 | 2 | 129.5 | 1 | 0.14585 | 1.56 |
| A1ZM044683_X_AT | NC0174062 | 2 | 129.5 | NC0199713 | 2 | 139.5 | 0 | −0.09392 | 1.01 |
| A1ZM044683_X_AT | NC0174062 | 2 | 129.5 | NC0187941 | 4 | 121 | 0 | −0.08747 | 0.94 |
| A1ZM044683_X_AT | NC0174062 | 2 | 129.5 | NC0173919 | 4 | 142.6 | 0 | −0.07772 | 0.83 |
| A1ZM044683_X_AT | NC0174062 | 2 | 129.5 | NC0199707 | 4 | 144 | 0 | −0.07868 | 0.84 |
| A1ZM044683_X_AT | NC0174062 | 2 | 129.5 | NC0199421 | 4 | 156.3 | 0 | 0.07624 | 0.82 |
| A1ZM044683_X_AT | NC0174062 | 2 | 129.5 | NC0199468 | 4 | 173.5 | 0 | −0.09507 | 1.02 |
| A1ZM044683_X_AT | NC0174062 | 2 | 129.5 | NC0020611 | 7 | 10 | 0 | −0.09100 | 0.98 |
| A1ZM044683_X_AT | NC0174062 | 2 | 129.5 | NC0200123 | 7 | 107.2 | 0 | 0.07672 | 0.82 |
| A1ZM045717_AT | NC0200185 | 7 | 147.7 | NC0013275 | 2 | 49.7 | 0 | −0.09518 | 0.89 |
| A1ZM045717_AT | NC0200185 | 7 | 147.7 | NC0173731 | 2 | 138.4 | 0 | −0.09861 | 0.93 |
| A1ZM045717_AT | NC0200185 | 7 | 147.7 | NC0003536 | 3 | 30.9 | 0 | −0.11558 | 1.09 |
| A1ZM045717_AT | NC0200185 | 7 | 147.7 | NC0199853 | 3 | 81.6 | 0 | 0.10200 | 0.96 |
| A1ZM045717_AT | NC0200185 | 7 | 147.7 | NC0015965 | 3 | 140.8 | 0 | −0.11562 | 1.09 |
| A1ZM045717_AT | NC0200185 | 7 | 147.7 | NC0174066 | 3 | 168.4 | 0 | 0.10456 | 0.98 |
| A1ZM045717_AT | NC0200185 | 7 | 147.7 | NC0200165 | 6 | 99.9 | 0 | 0.10037 | 0.94 |
| A1ZM045717_AT | NC0200185 | 7 | 147.7 | NC0195223 | 6 | 118.6 | 0 | −0.09872 | 0.93 |
| A1ZM045717_AT | NC0200185 | 7 | 147.7 | NC0200185 | 7 | 147.7 | 1 | −0.14206 | 1.34 |
| A1ZM045717_AT | NC0200185 | 7 | 147.7 | NC0173530 | 9 | 78.1 | 0 | 0.09117 | 0.86 |
| A1ZM045717_AT | NC0200185 | 7 | 147.7 | NC0011002 | 10 | 66.5 | 0 | −0.09638 | 0.91 |
| A1ZM049194_AT | NC0033320 | 2 | 173.6 | NC0188455 | 1 | 113.3 | 0 | −0.15498 | 1.03 |
| A1ZM049194_AT | NC0033320 | 2 | 173.6 | NC0199986 | 1 | 113.3 | 0 | −0.13866 | 0.92 |
| A1ZM049194_AT | NC0033320 | 2 | 173.6 | NC0033320 | 2 | 173.6 | 1 | 0.17864 | 1.18 |
| A1ZM049194_AT | NC0033320 | 2 | 173.6 | NC0199736 | 3 | 187.4 | 0 | 0.13749 | 0.91 |
| A1ZM049194_AT | NC0033320 | 2 | 173.6 | NC0199388 | 7 | 62.8 | 0 | 0.15072 | 1.00 |
| A1ZM049194_AT | NC0033320 | 2 | 173.6 | NC0035106 | 8 | 151.4 | 0 | −0.13989 | 0.93 |
| A1ZM049194_AT | NC0033320 | 2 | 173.6 | NC0200107 | 9 | 65.9 | 0 | 0.15087 | 1.00 |
| A1ZM049860_AT | NC0033526 | 8 | 153.1 | NC0199555 | 1 | 34.4 | 0 | −0.04466 | 0.80 |
| A1ZM049860_AT | NC0033526 | 8 | 153.1 | NC0036726 | 1 | 35.2 | 0 | −0.04682 | 0.84 |
| A1ZM049860_AT | NC0033526 | 8 | 153.1 | NC0199461 | 1 | 176.7 | 0 | −0.05193 | 0.93 |
| A1ZM049860_AT | NC0033526 | 8 | 153.1 | NC0196949 | 2 | 43.4 | 0 | −0.05155 | 0.92 |
| A1ZM049860_AT | NC0033526 | 8 | 153.1 | NC0000399 | 3 | 121.1 | 0 | −0.04823 | 0.87 |
| A1ZM049860_AT | NC0033526 | 8 | 153.1 | NC0112924 | 3 | 145.3 | 0 | −0.05231 | 0.94 |
| A1ZM049860_AT | NC0033526 | 8 | 153.1 | NC0199485 | 3 | 175.1 | 0 | 0.04549 | 0.82 |
| A1ZM049860_AT | NC0033526 | 8 | 153.1 | NC0189286 | 4 | 177 | 0 | −0.05817 | 1.04 |
| A1ZM049860_AT | NC0033526 | 8 | 153.1 | NC0199826 | 4 | 190.8 | 0 | −0.05297 | 0.95 |
| A1ZM049860_AT | NC0033526 | 8 | 153.1 | NC0199675 | 5 | 45.9 | 0 | −0.04764 | 0.85 |
| A1ZM049860_AT | NC0033526 | 8 | 153.1 | NC0199293 | 5 | 62.9 | 0 | −0.06489 | 1.16 |
| A1ZM049860_AT | NC0033526 | 8 | 153.1 | NC0199447 | 5 | 80.6 | 0 | −0.05135 | 0.92 |
| A1ZM049860_AT | NC0033526 | 8 | 153.1 | NC0194693 | 5 | 89.9 | 0 | −0.05182 | 0.93 |
| A1ZM049860_AT | NC0033526 | 8 | 153.1 | NC0200165 | 6 | 99.9 | 0 | 0.05040 | 0.90 |
| A1ZM049860_AT | NC0033526 | 8 | 153.1 | NC0195223 | 6 | 118.6 | 0 | −0.06379 | 1.14 |
| A1ZM049860_AT | NC0033526 | 8 | 153.1 | NC0199409 | 6 | 118.6 | 0 | 0.06106 | 1.10 |
| A1ZM049860_AT | NC0033526 | 8 | 153.1 | NC0194645 | 8 | 76.5 | 0 | −0.04655 | 0.84 |
| A1ZM049860_AT | NC0033526 | 8 | 153.1 | NC0199730 | 8 | 79.6 | 0 | −0.04895 | 0.88 |
| A1ZM049860_AT | NC0033526 | 8 | 153.1 | NC0199563 | 8 | 115.6 | 0 | 0.06209 | 1.11 |
| A1ZM049860_AT | NC0033526 | 8 | 153.1 | NC0035106 | 8 | 151.4 | 0 | 0.06365 | 1.14 |
| A1ZM049860_AT | NC0033526 | 8 | 153.1 | NC0033526 | 8 | 153.1 | 1 | −0.08533 | 1.53 |
| A1ZM049860_AT | NC0033526 | 8 | 153.1 | NC0014545 | 8 | 155.1 | 0 | −0.06725 | 1.21 |
| A1ZM049860_AT | NC0033526 | 8 | 153.1 | NC0056940 | 9 | 0.4 | 0 | 0.04689 | 0.84 |
| A1ZM052424_S_AT | NC0173400 | 1 | 32.2 | NC0173400 | 1 | 32.2 | 1 | 0.20604 | 1.23 |
| A1ZM052424_S_AT | NC0173400 | 1 | 32.2 | NC0200048 | 1 | 230.2 | 0 | −0.15107 | 0.90 |
| A1ZM052424_S_AT | NC0173400 | 1 | 32.2 | NC0199373 | 4 | 146.5 | 0 | −0.15848 | 0.95 |
| A1ZM052424_S_AT | NC0173400 | 1 | 32.2 | NC0199771 | 4 | 147.3 | 0 | −0.16240 | 0.97 |
| A1ZM052424_S_AT | NC0173400 | 1 | 32.2 | NC0200120 | 5 | 75.4 | 0 | 0.17627 | 1.05 |
| A1ZM052424_S_AT | NC0173400 | 1 | 32.2 | NC0173900 | 6 | 38.4 | 0 | −0.15573 | 0.93 |
| A1ZM052424_S_AT | NC0173400 | 1 | 32.2 | NC0010740 | 9 | 0 | 0 | 0.16726 | 1.00 |
| A1ZM052424_S_AT | NC0173400 | 1 | 32.2 | NC0174063 | 9 | 60.8 | 0 | −0.15674 | 0.94 |
| A1ZM053031_S_AT | NC0200156 | 3 | 96.1 | NC0200156 | 3 | 96.1 | 1 | −0.23808 | 1.08 |
| A1ZM053031_S_AT | NC0200156 | 3 | 96.1 | NC0199694 | 4 | 142.6 | 0 | −0.21371 | 0.97 |
| A1ZM053031_S_AT | NC0200156 | 3 | 96.1 | NC0199470 | 5 | 2 | 0 | −0.20944 | 0.95 |
| A1ZM055950_S_AT | NC0173484 | 1 | 103.2 | NC0032745 | 1 | 94.5 | 0 | 0.12701 | 1.02 |
| A1ZM055950_S_AT | NC0173484 | 1 | 103.2 | NC0173334 | 1 | 102.8 | 0 | −0.12943 | 1.04 |
| A1ZM055950_S_AT | NC0173484 | 1 | 103.2 | NC0173484 | 1 | 103.2 | 1 | −0.13569 | 1.09 |
| A1ZM055950_S_AT | NC0173484 | 1 | 103.2 | NC0199986 | 1 | 113.3 | 0 | −0.11593 | 0.93 |
| A1ZM055950_S_AT | NC0173484 | 1 | 103.2 | NC0009470 | 3 | 88 | 0 | 0.11345 | 0.91 |
| A1ZM056031_AT | NC0199589 | 3 | 91.5 | NC0173400 | 1 | 32.2 | 0 | −0.07378 | 0.82 |
| A1ZM056031_AT | NC0199589 | 3 | 91.5 | NC0032745 | 1 | 94.5 | 0 | 0.07942 | 0.88 |
| A1ZM056031_AT | NC0199589 | 3 | 91.5 | NC0173334 | 1 | 102.8 | 0 | −0.07499 | 0.83 |
| A1ZM056031_AT | NC0199589 | 3 | 91.5 | NC0173484 | 1 | 103.2 | 0 | −0.07676 | 0.85 |
| A1ZM056031_AT | NC0199589 | 3 | 91.5 | NC0199986 | 1 | 113.3 | 0 | −0.10327 | 1.14 |
| A1ZM056031_AT | NC0199589 | 3 | 91.5 | NC0199491 | 1 | 183.9 | 0 | −0.09474 | 1.05 |
| A1ZM056031_AT | NC0199589 | 3 | 91.5 | NC0200135 | 1 | 188.2 | 0 | −0.07811 | 0.86 |
| A1ZM056031_AT | NC0199589 | 3 | 91.5 | NC0188030 | 1 | 207.9 | 0 | −0.08376 | 0.93 |
| A1ZM056031_AT | NC0199589 | 3 | 91.5 | NC0173436 | 3 | 86 | 0 | 0.08082 | 0.89 |
| A1ZM056031_AT | NC0199589 | 3 | 91.5 | NC0199522 | 3 | 90.3 | 0 | 0.07372 | 0.81 |
| A1ZM056031_AT | NC0199589 | 3 | 91.5 | NC0199589 | 3 | 91.5 | 1 | −0.14178 | 1.57 |
| A1ZM056031_AT | NC0199589 | 3 | 91.5 | NC0199676 | 3 | 91.5 | 0 | −0.09551 | 1.06 |
| A1ZM056031_AT | NC0199589 | 3 | 91.5 | NC0200156 | 3 | 96.1 | 0 | −0.10127 | 1.12 |
| A1ZM056031_AT | NC0199589 | 3 | 91.5 | NC0015965 | 3 | 140.8 | 0 | 0.09528 | 1.05 |
| A1ZM056031_AT | NC0199589 | 3 | 91.5 | NC0199794 | 3 | 146 | 0 | 0.09071 | 1.00 |
| A1ZM056031_AT | NC0199589 | 3 | 91.5 | NC0190032 | 3 | 147.8 | 0 | 0.09035 | 1.00 |
| A1ZM056031_AT | NC0199589 | 3 | 91.5 | NC0188678 | 3 | 201.5 | 0 | 0.07961 | 0.88 |
| A1ZM056158_AT | NC0173626 | 8 | 96.8 | NC0173858 | 1 | 43.8 | 0 | 0.04889 | 0.85 |
| A1ZM056158_AT | NC0173626 | 8 | 96.8 | NC0199454 | 1 | 136.7 | 0 | 0.04976 | 0.86 |
| A1ZM056158_AT | NC0173626 | 8 | 96.8 | NC0199461 | 1 | 176.7 | 0 | −0.05268 | 0.91 |
| A1ZM056158_AT | NC0173626 | 8 | 96.8 | NC0013275 | 2 | 49.7 | 0 | −0.05459 | 0.94 |
| A1ZM056158_AT | NC0173626 | 8 | 96.8 | NC0200126 | 3 | 81.8 | 0 | −0.04747 | 0.82 |
| A1ZM056158_AT | NC0173626 | 8 | 96.8 | NC0112481 | 3 | 183.6 | 0 | 0.05083 | 0.88 |
| A1ZM056158_AT | NC0173626 | 8 | 96.8 | NC0195163 | 3 | 216.7 | 0 | 0.05444 | 0.94 |
| A1ZM056158_AT | NC0173626 | 8 | 96.8 | NC0200106 | 5 | 124.4 | 0 | −0.04846 | 0.84 |
| A1ZM056158_AT | NC0173626 | 8 | 96.8 | NC0079526 | 5 | 125 | 0 | −0.04863 | 0.84 |
| A1ZM056158_AT | NC0173626 | 8 | 96.8 | NC0107877 | 5 | 132.7 | 0 | 0.05397 | 0.93 |
| A1ZM056158_AT | NC0173626 | 8 | 96.8 | NC0036529 | 6 | 43.2 | 0 | 0.06204 | 1.07 |
| A1ZM056158_AT | NC0173626 | 8 | 96.8 | NC0199443 | 8 | 72.9 | 0 | −0.05580 | 0.97 |
| A1ZM056158_AT | NC0173626 | 8 | 96.8 | NC0194645 | 8 | 76.5 | 0 | −0.05995 | 1.04 |
| A1ZM056158_AT | NC0173626 | 8 | 96.8 | NC0199730 | 8 | 79.6 | 0 | −0.05925 | 1.03 |
| A1ZM056158_AT | NC0173626 | 8 | 96.8 | NC0199735 | 8 | 84.4 | 0 | −0.07448 | 1.29 |
| A1ZM056158_AT | NC0173626 | 8 | 96.8 | NC0200184 | 8 | 91.1 | 0 | −0.05010 | 0.87 |
| A1ZM056158_AT | NC0173626 | 8 | 96.8 | NC0199860 | 8 | 93.9 | 0 | −0.06080 | 1.05 |
| A1ZM056158_AT | NC0173626 | 8 | 96.8 | NC0199770 | 8 | 96.2 | 0 | −0.07017 | 1.21 |
| A1ZM056158_AT | NC0173626 | 8 | 96.8 | NC0199896 | 8 | 96.4 | 0 | 0.06574 | 1.14 |
| A1ZM056158_AT | NC0173626 | 8 | 96.8 | NC0173626 | 8 | 96.8 | 1 | −0.07939 | 1.37 |
| A1ZM056158_AT | NC0173626 | 8 | 96.8 | NC0111472 | 8 | 103.3 | 0 | 0.05842 | 1.01 |
| A1ZM056158_AT | NC0173626 | 8 | 96.8 | NC0148189 | 9 | 91 | 0 | 0.05168 | 0.89 |
| A1ZM057238_AT | NC0199958 | 6 | 142 | NC0144205 | 1 | 38.5 | 0 | −0.06526 | 1.11 |
| A1ZM057238_AT | NC0199958 | 6 | 142 | NC0200044 | 1 | 47.5 | 0 | 0.05369 | 0.91 |
| A1ZM057238_AT | NC0199958 | 6 | 142 | NC0199346 | 2 | 50.8 | 0 | −0.05372 | 0.91 |
| A1ZM057238_AT | NC0199958 | 6 | 142 | NC0002630 | 2 | 54.1 | 0 | 0.05891 | 1.00 |
| A1ZM057238_AT | NC0199958 | 6 | 142 | NC0199853 | 3 | 81.6 | 0 | 0.05222 | 0.89 |
| A1ZM057238_AT | NC0199958 | 6 | 142 | NC0015965 | 3 | 140.8 | 0 | −0.05865 | 0.99 |
| A1ZM057238_AT | NC0199958 | 6 | 142 | NC0112924 | 3 | 145.3 | 0 | −0.05626 | 0.95 |
| A1ZM057238_AT | NC0199958 | 6 | 142 | NC0199794 | 3 | 146 | 0 | −0.05461 | 0.93 |
| A1ZM057238_AT | NC0199958 | 6 | 142 | NC0174066 | 3 | 168.4 | 0 | 0.05564 | 0.94 |
| A1ZM057238_AT | NC0199958 | 6 | 142 | NC0190159 | 3 | 168.4 | 0 | 0.05467 | 0.93 |
| A1ZM057238_AT | NC0199958 | 6 | 142 | NC0173452 | 5 | 177.7 | 0 | 0.07271 | 1.23 |
| A1ZM057238_AT | NC0199958 | 6 | 142 | NC0200165 | 6 | 99.9 | 0 | 0.05441 | 0.92 |
| A1ZM057238_AT | NC0199958 | 6 | 142 | NC0199958 | 6 | 142 | 1 | −0.08368 | 1.42 |
| A1ZM057238_AT | NC0199958 | 6 | 142 | NC0194906 | 6 | 144.8 | 0 | 0.06253 | 1.06 |
| A1ZM057238_AT | NC0199958 | 6 | 142 | NC0199312 | 7 | 65.9 | 0 | 0.06325 | 1.07 |
| A1ZM057238_AT | NC0199958 | 6 | 142 | NC0000129 | 8 | 16.5 | 0 | −0.05206 | 0.88 |
| A1ZM057238_AT | NC0199958 | 6 | 142 | NC0049890 | 8 | 41.2 | 0 | 0.06193 | 1.05 |
| A1ZM057238_AT | NC0199958 | 6 | 142 | NC0199834 | 8 | 84 | 0 | −0.05193 | 0.88 |
| A1ZM057238_AT | NC0199958 | 6 | 142 | NC0199464 | 9 | 66.5 | 0 | 0.05383 | 0.91 |
| A1ZM057238_AT | NC0199958 | 6 | 142 | NC0002876 | 9 | 66.6 | 0 | 0.05434 | 0.92 |
| A1ZM057238_AT | NC0199958 | 6 | 142 | NC0107256 | 9 | 68.5 | 0 | −0.06930 | 1.18 |
| A1ZM057238_AT | NC0199958 | 6 | 142 | NC0173799 | 9 | 68.5 | 0 | 0.06056 | 1.03 |
| A1ZM057238_AT | NC0199958 | 6 | 142 | NC0020872 | 9 | 76.3 | 0 | −0.05208 | 0.88 |
| A1ZM057238_AT | NC0199958 | 6 | 142 | NC0173530 | 9 | 78.1 | 0 | 0.05862 | 0.99 |
| A1ZM057238_AT | NC0199958 | 6 | 142 | NC0199722 | 9 | 79.6 | 0 | 0.05288 | 0.90 |
| A1ZM057238_AT | NC0199958 | 6 | 142 | NC0028613 | 9 | 94.6 | 0 | −0.05356 | 0.91 |
| A1ZM057468_AT | NC0036529 | 6 | 43.2 | NC0175336 | 1 | 43.5 | 0 | 0.06387 | 0.98 |
| A1ZM057468_AT | NC0036529 | 6 | 43.2 | NC0173858 | 1 | 43.8 | 0 | −0.06943 | 1.07 |
| A1ZM057468_AT | NC0036529 | 6 | 43.2 | NC0200188 | 1 | 44.1 | 0 | 0.06204 | 0.96 |
| A1ZM057468_AT | NC0036529 | 6 | 43.2 | NC0199760 | 1 | 179.1 | 0 | 0.05950 | 0.92 |
| A1ZM057468_AT | NC0036529 | 6 | 43.2 | NC0173409 | 2 | 32.7 | 0 | −0.07140 | 1.10 |
| A1ZM057468_AT | NC0036529 | 6 | 43.2 | NC0013275 | 2 | 49.7 | 0 | 0.06516 | 1.00 |
| A1ZM057468_AT | NC0036529 | 6 | 43.2 | NC0027262 | 2 | 57.3 | 0 | −0.05904 | 0.91 |
| A1ZM057468_AT | NC0036529 | 6 | 43.2 | NC0009470 | 3 | 88 | 0 | −0.05992 | 0.92 |
| A1ZM057468_AT | NC0036529 | 6 | 43.2 | NC0015965 | 3 | 140.8 | 0 | 0.06627 | 1.02 |
| A1ZM057468_AT | NC0036529 | 6 | 43.2 | NC0199794 | 3 | 146 | 0 | 0.06215 | 0.96 |
| A1ZM057468_AT | NC0036529 | 6 | 43.2 | NC0190032 | 3 | 147.8 | 0 | 0.06144 | 0.95 |
| A1ZM057468_AT | NC0036529 | 6 | 43.2 | NC0200083 | 4 | 84.9 | 0 | −0.05970 | 0.92 |
| A1ZM057468_AT | NC0036529 | 6 | 43.2 | NC0199945 | 6 | 39.4 | 0 | 0.07298 | 1.12 |
| A1ZM057468_AT | NC0036529 | 6 | 43.2 | NC0036529 | 6 | 43.2 | 1 | −0.07957 | 1.23 |
| A1ZM057468_AT | NC0036529 | 6 | 43.2 | NC0020611 | 7 | 10 | 0 | −0.06997 | 1.08 |
| A1ZM057468_AT | NC0036529 | 6 | 43.2 | NC0199685 | 7 | 28.6 | 0 | 0.06089 | 0.94 |
| A1ZM057468_AT | NC0036529 | 6 | 43.2 | NC0199984 | 7 | 60.4 | 0 | −0.06360 | 0.98 |
| A1ZM057468_AT | NC0036529 | 6 | 43.2 | NC0199889 | 7 | 99.4 | 0 | 0.06073 | 0.94 |
| A1ZM057468_AT | NC0036529 | 6 | 43.2 | NC0173530 | 9 | 78.1 | 0 | −0.05959 | 0.92 |
| A1ZM057468_AT | NC0036529 | 6 | 43.2 | NC0199722 | 9 | 79.6 | 0 | −0.06904 | 1.06 |
| A1ZM057468_AT | NC0036529 | 6 | 43.2 | NC0200144 | 9 | 110.4 | 0 | −0.06207 | 0.96 |
| A1ZM057856_AT | NC0173859 | 7 | 96.8 | NC0200044 | 1 | 47.5 | 0 | 0.05103 | 0.99 |
| A1ZM057856_AT | NC0173859 | 7 | 96.8 | NC0143873 | 1 | 52.6 | 0 | 0.04272 | 0.83 |
| A1ZM057856_AT | NC0173859 | 7 | 96.8 | NC0200213 | 1 | 67.4 | 0 | 0.05713 | 1.11 |
| A1ZM057856_AT | NC0173859 | 7 | 96.8 | NC0173436 | 3 | 86 | 0 | −0.05152 | 1.00 |
| A1ZM057856_AT | NC0173859 | 7 | 96.8 | NC0199589 | 3 | 91.5 | 0 | 0.05309 | 1.03 |
| A1ZM057856_AT | NC0173859 | 7 | 96.8 | NC0199676 | 3 | 91.5 | 0 | 0.04799 | 0.93 |
| A1ZM057856_AT | NC0173859 | 7 | 96.8 | NC0199814 | 3 | 208.5 | 0 | −0.04476 | 0.87 |
| A1ZM057856_AT | NC0173859 | 7 | 96.8 | NC0199657 | 4 | 0.5 | 0 | −0.04300 | 0.83 |
| A1ZM057856_AT | NC0173859 | 7 | 96.8 | NC0199824 | 4 | 13.6 | 0 | 0.04271 | 0.83 |
| A1ZM057856_AT | NC0173859 | 7 | 96.8 | NC0199781 | 4 | 82.7 | 0 | −0.05716 | 1.11 |
| A1ZM057856_AT | NC0173859 | 7 | 96.8 | NC0189552 | 5 | 63.8 | 0 | −0.04908 | 0.95 |
| A1ZM057856_AT | NC0173859 | 7 | 96.8 | NC0200171 | 5 | 73.2 | 0 | 0.04993 | 0.97 |
| A1ZM057856_AT | NC0173859 | 7 | 96.8 | NC0078090 | 5 | 141.8 | 0 | −0.04414 | 0.86 |
| A1ZM057856_AT | NC0173859 | 7 | 96.8 | NC0042059 | 5 | 143.2 | 0 | −0.04916 | 0.95 |
| A1ZM057856_AT | NC0173859 | 7 | 96.8 | NC0173843 | 5 | 143.2 | 0 | 0.04613 | 0.89 |
| A1ZM057856_AT | NC0173859 | 7 | 96.8 | NC0199422 | 6 | 41.1 | 0 | 0.05016 | 0.97 |
| A1ZM057856_AT | NC0173859 | 7 | 96.8 | NC0200199 | 7 | 12.4 | 0 | −0.04304 | 0.83 |
| A1ZM057856_AT | NC0173859 | 7 | 96.8 | NC0199723 | 7 | 40.4 | 0 | −0.04648 | 0.90 |
| A1ZM057856_AT | NC0173859 | 7 | 96.8 | NC0199648 | 7 | 62.5 | 0 | 0.05128 | 0.99 |
| A1ZM057856_AT | NC0173859 | 7 | 96.8 | NC0173493 | 7 | 62.9 | 0 | 0.04739 | 0.92 |
| A1ZM057856_AT | NC0173859 | 7 | 96.8 | NC0200064 | 7 | 65.3 | 0 | 0.04348 | 0.84 |
| A1ZM057856_AT | NC0173859 | 7 | 96.8 | NC0200207 | 7 | 84.1 | 0 | −0.05408 | 1.05 |
| A1ZM057856_AT | NC0173859 | 7 | 96.8 | NC0008799 | 7 | 87 | 0 | 0.06288 | 1.22 |
| A1ZM057856_AT | NC0173859 | 7 | 96.8 | NC0199354 | 7 | 87.4 | 0 | 0.06461 | 1.25 |
| A1ZM057856_AT | NC0173859 | 7 | 96.8 | NC0173859 | 7 | 96.8 | 1 | −0.07996 | 1.55 |
| A1ZM057856_AT | NC0173859 | 7 | 96.8 | NC0015314 | 7 | 99 | 0 | 0.04511 | 0.87 |
| A1ZM057856_AT | NC0173859 | 7 | 96.8 | NC0071001 | 7 | 99.4 | 0 | −0.05597 | 1.09 |
| A1ZM057856_AT | NC0173859 | 7 | 96.8 | NC0199889 | 7 | 99.4 | 0 | −0.05885 | 1.14 |
| A1ZM057856_AT | NC0173859 | 7 | 96.8 | NC0199614 | 7 | 108.4 | 0 | −0.04539 | 0.88 |
| A1ZM057856_AT | NC0173859 | 7 | 96.8 | NC0199339 | 7 | 150.8 | 0 | 0.05037 | 0.98 |
| A1ZM058469_AT | NC0173436 | 3 | 86 | NC0199803 | 3 | 85.6 | 0 | −0.14536 | 0.85 |
| A1ZM058469_AT | NC0173436 | 3 | 86 | NC0173436 | 3 | 86 | 1 | −0.21419 | 1.25 |
| A1ZM058469_AT | NC0173436 | 3 | 86 | NC0199947 | 3 | 86.4 | 0 | 0.18693 | 1.09 |
| A1ZM058469_AT | NC0173436 | 3 | 86 | NC0199372 | 3 | 87.1 | 0 | 0.14177 | 0.83 |
| A1ZM058469_AT | NC0173436 | 3 | 86 | NC0199589 | 3 | 91.5 | 0 | 0.13787 | 0.80 |
| A1ZM058469_AT | NC0173436 | 3 | 86 | NC0199676 | 3 | 91.5 | 0 | 0.18896 | 1.10 |
| A1ZM061580_AT | NC0199555 | 1 | 34.4 | NC0173400 | 1 | 32.2 | 0 | −0.09447 | 0.88 |
| A1ZM061580_AT | NC0199555 | 1 | 34.4 | NC0173332 | 1 | 33.3 | 0 | −0.10510 | 0.98 |
| A1ZM061580_AT | NC0199555 | 1 | 34.4 | NC0199555 | 1 | 34.4 | 1 | 0.14385 | 1.34 |
| A1ZM061580_AT | NC0199555 | 1 | 34.4 | NC0035426 | 1 | 34.5 | 0 | −0.09403 | 0.88 |
| A1ZM061580_AT | NC0199555 | 1 | 34.4 | NC0036726 | 1 | 35.2 | 0 | 0.10530 | 0.98 |
| A1ZM061580_AT | NC0199555 | 1 | 34.4 | NC0173919 | 4 | 142.6 | 0 | −0.10371 | 0.97 |
| A1ZM061580_AT | NC0199555 | 1 | 34.4 | NC0199575 | 4 | 142.6 | 0 | 0.11151 | 1.04 |
| A1ZM061580_AT | NC0199555 | 1 | 34.4 | NC0199739 | 5 | 62.9 | 0 | 0.10562 | 0.99 |
| A1ZM061580_AT | NC0199555 | 1 | 34.4 | NC0079519 | 5 | 63.1 | 0 | −0.11113 | 1.04 |
| A1ZM061580_AT | NC0199555 | 1 | 34.4 | NC0194397 | 6 | 38.4 | 0 | 0.09573 | 0.89 |
| A1ZM061580_AT | NC0199555 | 1 | 34.4 | NC0199860 | 8 | 93.9 | 0 | 0.09696 | 0.91 |
| A1ZM061606_X_AT | NC0013275 | 2 | 49.7 | NC0199461 | 1 | 176.7 | 0 | −0.09419 | 1.03 |
| A1ZM061606_X_AT | NC0013275 | 2 | 49.7 | NC0013275 | 2 | 49.7 | 1 | −0.11516 | 1.26 |
| A1ZM061606_X_AT | NC0013275 | 2 | 49.7 | NC0199346 | 2 | 50.8 | 0 | −0.08002 | 0.87 |
| A1ZM061606_X_AT | NC0013275 | 2 | 49.7 | NC0015965 | 3 | 140.8 | 0 | −0.09671 | 1.05 |
| A1ZM061606_X_AT | NC0013275 | 2 | 49.7 | NC0199794 | 3 | 146 | 0 | −0.08644 | 0.94 |
| A1ZM061606_X_AT | NC0013275 | 2 | 49.7 | NC0173962 | 6 | 96.7 | 0 | −0.09175 | 1.00 |
| A1ZM061606_X_AT | NC0013275 | 2 | 49.7 | NC0199735 | 8 | 84.4 | 0 | −0.07938 | 0.87 |
| A1ZM061606_X_AT | NC0013275 | 2 | 49.7 | NC0174094 | 8 | 91.1 | 0 | −0.09285 | 1.01 |
| A1ZM061606_X_AT | NC0013275 | 2 | 49.7 | NC0199722 | 9 | 79.6 | 0 | 0.08361 | 0.91 |
| A1ZM062662_AT | NC0195223 | 6 | 118.6 | NC0199415 | 2 | 84.7 | 0 | 0.11662 | 1.00 |
| A1ZM062662_AT | NC0195223 | 6 | 118.6 | NC0174061 | 2 | 118.1 | 0 | −0.11292 | 0.97 |
| A1ZM062662_AT | NC0195223 | 6 | 118.6 | NC0174062 | 2 | 129.5 | 0 | −0.11671 | 1.00 |
| A1ZM062662_AT | NC0195223 | 6 | 118.6 | NC0173731 | 2 | 138.4 | 0 | −0.11862 | 1.01 |
| A1ZM062662_AT | NC0195223 | 6 | 118.6 | NC0173436 | 3 | 86 | 0 | −0.11666 | 1.00 |
| A1ZM062662_AT | NC0195223 | 6 | 118.6 | NC0199741 | 3 | 125.7 | 0 | −0.11365 | 0.97 |
| A1ZM062662_AT | NC0195223 | 6 | 118.6 | NC0195223 | 6 | 118.6 | 1 | −0.12874 | 1.10 |
| A1ZM062662_AT | NC0195223 | 6 | 118.6 | NC0199409 | 6 | 118.6 | 0 | 0.11429 | 0.98 |
| A1ZM062662_AT | NC0195223 | 6 | 118.6 | NC0173476 | 7 | 44.2 | 0 | −0.11488 | 0.98 |
| A1ZM062662_AT | NC0195223 | 6 | 118.6 | NC0200185 | 7 | 147.7 | 0 | −0.12476 | 1.07 |
| A1ZM062662_AT | NC0195223 | 6 | 118.6 | NC0199722 | 9 | 79.6 | 0 | 0.11213 | 0.96 |
| A1ZM062662_AT | NC0195223 | 6 | 118.6 | NC0011002 | 10 | 66.5 | 0 | −0.11237 | 0.96 |
| A1ZM063309_AT | NC0173951 | 1 | 229.4 | NC0173951 | 1 | 229.4 | 1 | 0.14379 | 1.15 |
| A1ZM063309_AT | NC0173951 | 1 | 229.4 | NC0013275 | 2 | 49.7 | 0 | −0.11625 | 0.93 |
| A1ZM063309_AT | NC0173951 | 1 | 229.4 | NC0199346 | 2 | 50.8 | 0 | −0.13208 | 1.05 |
| A1ZM063309_AT | NC0173951 | 1 | 229.4 | NC0174066 | 3 | 168.4 | 0 | 0.10976 | 0.88 |
| A1ZM063309_AT | NC0173951 | 1 | 229.4 | NC0199750 | 6 | 81.6 | 0 | −0.12313 | 0.98 |
| A1ZM063309_AT | NC0173951 | 1 | 229.4 | NC0199354 | 7 | 87.4 | 0 | 0.11748 | 0.94 |
| A1ZM063309_AT | NC0173951 | 1 | 229.4 | NC0200185 | 7 | 147.7 | 0 | −0.13465 | 1.07 |
| A1ZM063309_AT | NC0173951 | 1 | 229.4 | NC0049890 | 8 | 41.2 | 0 | 0.13420 | 1.07 |
| A1ZM063309_AT | NC0173951 | 1 | 229.4 | NC0023268 | 9 | 84.5 | 0 | 0.11409 | 0.91 |
| A1ZM063309_AT | NC0173951 | 1 | 229.4 | NC0014240 | 9 | 103.5 | 0 | −0.13391 | 1.07 |
| A1ZM063309_AT | NC0173951 | 1 | 229.4 | NC0030512 | 9 | 103.6 | 0 | −0.11451 | 0.91 |
| A1ZM067500_AT | NC0013275 | 2 | 49.7 | NC0199491 | 1 | 183.9 | 0 | 0.07835 | 0.89 |
| A1ZM067500_AT | NC0013275 | 2 | 49.7 | NC0196949 | 2 | 43.4 | 0 | −0.08829 | 1.01 |
| A1ZM067500_AT | NC0013275 | 2 | 49.7 | NC0013275 | 2 | 49.7 | 1 | −0.12334 | 1.41 |
| A1ZM067500_AT | NC0013275 | 2 | 49.7 | NC0199346 | 2 | 50.8 | 0 | −0.07524 | 0.86 |
| A1ZM067500_AT | NC0013275 | 2 | 49.7 | NC0170324 | 2 | 94 | 0 | −0.07637 | 0.87 |
| A1ZM067500_AT | NC0013275 | 2 | 49.7 | NC0172302 | 2 | 94 | 0 | −0.08050 | 0.92 |
| A1ZM067500_AT | NC0013275 | 2 | 49.7 | NC0200140 | 2 | 99.3 | 0 | −0.07575 | 0.86 |
| A1ZM067500_AT | NC0013275 | 2 | 49.7 | NC0195038 | 3 | 74.2 | 0 | −0.07967 | 0.91 |
| A1ZM067500_AT | NC0013275 | 2 | 49.7 | NC0109315 | 3 | 77.4 | 0 | −0.07688 | 0.88 |
| A1ZM067500_AT | NC0013275 | 2 | 49.7 | NC0173436 | 3 | 86 | 0 | −0.08576 | 0.98 |
| A1ZM067500_AT | NC0013275 | 2 | 49.7 | NC0199794 | 3 | 146 | 0 | −0.09025 | 1.03 |
| A1ZM067500_AT | NC0013275 | 2 | 49.7 | NC0193457 | 3 | 216.6 | 0 | 0.07952 | 0.91 |
| A1ZM067500_AT | NC0013275 | 2 | 49.7 | NC0195223 | 6 | 118.6 | 0 | −0.11449 | 1.31 |
| A1ZM067500_AT | NC0013275 | 2 | 49.7 | NC0199409 | 6 | 118.6 | 0 | 0.09597 | 1.10 |
| A1ZM067500_AT | NC0013275 | 2 | 49.7 | NC0200064 | 7 | 65.3 | 0 | 0.09251 | 1.06 |
| A1ZM067500_AT | NC0013275 | 2 | 49.7 | NC0200039 | 8 | 55.3 | 0 | −0.07690 | 0.88 |
| A1ZM067500_AT | NC0013275 | 2 | 49.7 | NC0199722 | 9 | 79.6 | 0 | 0.08278 | 0.94 |
| A1ZM067740_X_AT | NC0014240 | 9 | 103.5 | NC0199346 | 2 | 50.8 | 0 | −0.10998 | 0.97 |
| A1ZM067740_X_AT | NC0014240 | 9 | 103.5 | NC0173350 | 8 | 104 | 0 | 0.09548 | 0.84 |
| A1ZM067740_X_AT | NC0014240 | 9 | 103.5 | NC0020872 | 9 | 76.3 | 0 | −0.10087 | 0.89 |
| A1ZM067740_X_AT | NC0014240 | 9 | 103.5 | NC0173530 | 9 | 78.1 | 0 | 0.11435 | 1.01 |
| A1ZM067740_X_AT | NC0014240 | 9 | 103.5 | NC0199722 | 9 | 79.6 | 0 | 0.11915 | 1.05 |
| A1ZM067740_X_AT | NC0014240 | 9 | 103.5 | NC0014240 | 9 | 103.5 | 1 | −0.13644 | 1.20 |
| A1ZM069545_AT | NC0199461 | 1 | 176.7 | NC0199461 | 1 | 176.7 | 1 | 0.05472 | 1.35 |
| A1ZM069545_AT | NC0199461 | 1 | 176.7 | NC0013275 | 2 | 49.7 | 0 | 0.04804 | 1.19 |
| A1ZM069545_AT | NC0199461 | 1 | 176.7 | NC0170324 | 2 | 94 | 0 | 0.04267 | 1.06 |
| A1ZM069545_AT | NC0199461 | 1 | 176.7 | NC0172302 | 2 | 94 | 0 | 0.04211 | 1.04 |
| A1ZM069545_AT | NC0199461 | 1 | 176.7 | NC0199297 | 2 | 100.2 | 0 | −0.04390 | 1.09 |
| A1ZM069545_AT | NC0199461 | 1 | 176.7 | NC0199568 | 2 | 114.7 | 0 | 0.03882 | 0.96 |
| A1ZM069545_AT | NC0199461 | 1 | 176.7 | NC0035094 | 2 | 116.9 | 0 | 0.05036 | 1.25 |
| A1ZM069545_AT | NC0199461 | 1 | 176.7 | NC0199511 | 2 | 121.3 | 0 | −0.03593 | 0.89 |
| A1ZM069545_AT | NC0199461 | 1 | 176.7 | NC0038699 | 2 | 121.8 | 0 | 0.04728 | 1.17 |
| A1ZM069545_AT | NC0199461 | 1 | 176.7 | NC0004901 | 2 | 131.6 | 0 | −0.03728 | 0.92 |
| A1ZM069545_AT | NC0199461 | 1 | 176.7 | NC0188475 | 2 | 131.6 | 0 | −0.03337 | 0.83 |
| A1ZM069545_AT | NC0199461 | 1 | 176.7 | NC0193437 | 2 | 131.6 | 0 | −0.03337 | 0.83 |
| A1ZM069545_AT | NC0199461 | 1 | 176.7 | NC0199851 | 2 | 165.2 | 0 | −0.03446 | 0.85 |
| A1ZM069545_AT | NC0199461 | 1 | 176.7 | NC0200126 | 3 | 81.8 | 0 | 0.03739 | 0.92 |
| A1ZM069545_AT | NC0199461 | 1 | 176.7 | NC0173790 | 3 | 83.2 | 0 | −0.03758 | 0.93 |
| A1ZM069545_AT | NC0199461 | 1 | 176.7 | NC0199763 | 3 | 86 | 0 | −0.03741 | 0.92 |
| A1ZM069545_AT | NC0199461 | 1 | 176.7 | NC0199947 | 3 | 86.4 | 0 | −0.03731 | 0.92 |
| A1ZM069545_AT | NC0199461 | 1 | 176.7 | NC0173739 | 3 | 116.9 | 0 | −0.03857 | 0.95 |
| A1ZM069545_AT | NC0199461 | 1 | 176.7 | NC0000399 | 3 | 121.1 | 0 | 0.05170 | 1.28 |
| A1ZM069545_AT | NC0199461 | 1 | 176.7 | NC0015965 | 3 | 140.8 | 0 | 0.03821 | 0.94 |
| A1ZM069545_AT | NC0199461 | 1 | 176.7 | NC0112924 | 3 | 145.3 | 0 | 0.03788 | 0.94 |
| A1ZM069545_AT | NC0199461 | 1 | 176.7 | NC0199794 | 3 | 146 | 0 | 0.03895 | 0.96 |
| A1ZM069545_AT | NC0199461 | 1 | 176.7 | NC0112481 | 3 | 183.6 | 0 | −0.03723 | 0.92 |
| A1ZM069545_AT | NC0199461 | 1 | 176.7 | NC0067577 | 4 | 59.3 | 0 | −0.03513 | 0.87 |
| A1ZM069545_AT | NC0199461 | 1 | 176.7 | NC0173568 | 4 | 150.5 | 0 | −0.03699 | 0.91 |
| A1ZM069545_AT | NC0199461 | 1 | 176.7 | NC0195197 | 5 | 66.6 | 0 | 0.03837 | 0.95 |
| A1ZM069545_AT | NC0199461 | 1 | 176.7 | NC0200171 | 5 | 73.2 | 0 | 0.04079 | 1.01 |
| A1ZM069545_AT | NC0199461 | 1 | 176.7 | NC0056874 | 5 | 73.5 | 0 | −0.03396 | 0.84 |
| A1ZM069545_AT | NC0199461 | 1 | 176.7 | NC0079526 | 5 | 125 | 0 | 0.03726 | 0.92 |
| A1ZM069545_AT | NC0199461 | 1 | 176.7 | NC0107877 | 5 | 132.7 | 0 | −0.03445 | 0.85 |
| A1ZM069545_AT | NC0199461 | 1 | 176.7 | NC0199915 | 6 | 84.9 | 0 | 0.03629 | 0.90 |
| A1ZM069545_AT | NC0199461 | 1 | 176.7 | NC0195223 | 6 | 118.6 | 0 | 0.03432 | 0.85 |
| A1ZM069545_AT | NC0199461 | 1 | 176.7 | NC0199409 | 6 | 118.6 | 0 | −0.03767 | 0.93 |
| A1ZM069545_AT | NC0199461 | 1 | 176.7 | NC0199443 | 8 | 72.9 | 0 | 0.04287 | 1.06 |
| A1ZM069545_AT | NC0199461 | 1 | 176.7 | NC0194645 | 8 | 76.5 | 0 | 0.05069 | 1.25 |
| A1ZM069545_AT | NC0199461 | 1 | 176.7 | NC0199730 | 8 | 79.6 | 0 | 0.04403 | 1.09 |
| A1ZM069545_AT | NC0199461 | 1 | 176.7 | NC0111472 | 8 | 103.3 | 0 | −0.04539 | 1.12 |
| A1ZM069545_AT | NC0199461 | 1 | 176.7 | NC0033526 | 8 | 153.1 | 0 | 0.04763 | 1.18 |
| A1ZM069545_AT | NC0199461 | 1 | 176.7 | NC0014545 | 8 | 155.1 | 0 | 0.03408 | 0.84 |
| A1ZM069545_AT | NC0199461 | 1 | 176.7 | NC0028613 | 9 | 94.6 | 0 | 0.04019 | 0.99 |
| A1ZM069545_AT | NC0199461 | 1 | 176.7 | NC0053108 | 10 | 61.2 | 0 | 0.03832 | 0.95 |
| A1ZM072906_AT | NC0199563 | 8 | 115.6 | NC0013275 | 2 | 49.7 | 0 | −0.12449 | 0.96 |
| A1ZM072906_AT | NC0199563 | 8 | 115.6 | NC0003536 | 3 | 30.9 | 0 | −0.13511 | 1.04 |
| A1ZM072906_AT | NC0199563 | 8 | 115.6 | NC0195223 | 6 | 118.6 | 0 | −0.13512 | 1.04 |
| A1ZM072906_AT | NC0199563 | 8 | 115.6 | NC0199409 | 6 | 118.6 | 0 | 0.12655 | 0.98 |
| A1ZM072906_AT | NC0199563 | 8 | 115.6 | NC0199958 | 6 | 142 | 0 | −0.13363 | 1.03 |
| A1ZM072906_AT | NC0199563 | 8 | 115.6 | NC0199312 | 7 | 65.9 | 0 | 0.12633 | 0.97 |
| A1ZM072906_AT | NC0199563 | 8 | 115.6 | NC0199563 | 8 | 115.6 | 1 | 0.13684 | 1.05 |
| A1ZM072906_AT | NC0199563 | 8 | 115.6 | NC0011002 | 10 | 66.5 | 0 | −0.11878 | 0.92 |
| A1ZM077940_X_AT | NC0193457 | 3 | 216.6 | NC0199738 | 2 | 28.8 | 0 | 0.05337 | 0.82 |
| A1ZM077940_X_AT | NC0193457 | 3 | 216.6 | NC0196898 | 2 | 33.5 | 0 | 0.06258 | 0.96 |
| A1ZM077940_X_AT | NC0193457 | 3 | 216.6 | NC0013275 | 2 | 49.7 | 0 | −0.05481 | 0.85 |
| A1ZM077940_X_AT | NC0193457 | 3 | 216.6 | NC0199851 | 2 | 165.2 | 0 | 0.06089 | 0.94 |
| A1ZM077940_X_AT | NC0193457 | 3 | 216.6 | NC0033320 | 2 | 173.6 | 0 | −0.05286 | 0.81 |
| A1ZM077940_X_AT | NC0193457 | 3 | 216.6 | NC0110714 | 2 | 190.1 | 0 | 0.05948 | 0.92 |
| A1ZM077940_X_AT | NC0193457 | 3 | 216.6 | NC0173436 | 3 | 86 | 0 | −0.05726 | 0.88 |
| A1ZM077940_X_AT | NC0193457 | 3 | 216.6 | NC0188678 | 3 | 201.5 | 0 | −0.08312 | 1.28 |
| A1ZM077940_X_AT | NC0193457 | 3 | 216.6 | NC0018073 | 3 | 201.6 | 0 | −0.07971 | 1.23 |
| A1ZM077940_X_AT | NC0193457 | 3 | 216.6 | NC0000189 | 3 | 215.3 | 0 | 0.07925 | 1.22 |
| A1ZM077940_X_AT | NC0193457 | 3 | 216.6 | NC0193457 | 3 | 216.6 | 1 | 0.08565 | 1.32 |
| A1ZM077940_X_AT | NC0193457 | 3 | 216.6 | NC0170468 | 3 | 216.7 | 0 | −0.06831 | 1.05 |
| A1ZM077940_X_AT | NC0193457 | 3 | 216.6 | NC0195163 | 3 | 216.7 | 0 | 0.06041 | 0.93 |
| A1ZM077940_X_AT | NC0193457 | 3 | 216.6 | NC0038211 | 4 | 141.1 | 0 | −0.06188 | 0.95 |
| A1ZM077940_X_AT | NC0193457 | 3 | 216.6 | NC0173628 | 4 | 144 | 0 | −0.06191 | 0.95 |
| A1ZM077940_X_AT | NC0193457 | 3 | 216.6 | NC0173518 | 4 | 152.5 | 0 | 0.06674 | 1.03 |
| A1ZM077940_X_AT | NC0193457 | 3 | 216.6 | NC0200099 | 5 | 171.1 | 0 | −0.05578 | 0.86 |
| A1ZM077940_X_AT | NC0193457 | 3 | 216.6 | NC0029612 | 6 | 57.8 | 0 | −0.06533 | 1.01 |
| A1ZM077940_X_AT | NC0193457 | 3 | 216.6 | NC0199409 | 6 | 118.6 | 0 | 0.05459 | 0.84 |
| A1ZM077940_X_AT | NC0193457 | 3 | 216.6 | NC0200186 | 6 | 131 | 0 | 0.05498 | 0.85 |
| A1ZM077940_X_AT | NC0193457 | 3 | 216.6 | NC0013445 | 6 | 131.2 | 0 | −0.05634 | 0.87 |
| A1ZM077940_X_AT | NC0193457 | 3 | 216.6 | NC0200199 | 7 | 12.4 | 0 | −0.06039 | 0.93 |
| A1ZM077940_X_AT | NC0193457 | 3 | 216.6 | NC0113218 | 8 | 75.2 | 0 | −0.08328 | 1.28 |
| A1ZM077940_X_AT | NC0193457 | 3 | 216.6 | NC0199574 | 10 | 45.2 | 0 | 0.05868 | 0.90 |
| A1ZM079744_AT | NC0013275 | 2 | 49.7 | NC0196949 | 2 | 43.4 | 0 | −0.06124 | 0.93 |
| A1ZM079744_AT | NC0013275 | 2 | 49.7 | NC0013275 | 2 | 49.7 | 1 | −0.09514 | 1.45 |
| A1ZM079744_AT | NC0013275 | 2 | 49.7 | NC0199346 | 2 | 50.8 | 0 | −0.05850 | 0.89 |
| A1ZM079744_AT | NC0013275 | 2 | 49.7 | NC0003536 | 3 | 30.9 | 0 | −0.05766 | 0.88 |
| A1ZM079744_AT | NC0013275 | 2 | 49.7 | NC0199741 | 3 | 125.7 | 0 | −0.05682 | 0.86 |
| A1ZM079744_AT | NC0013275 | 2 | 49.7 | NC0015965 | 3 | 140.8 | 0 | −0.08197 | 1.25 |
| A1ZM079744_AT | NC0013275 | 2 | 49.7 | NC0112924 | 3 | 145.3 | 0 | −0.07165 | 1.09 |
| A1ZM079744_AT | NC0013275 | 2 | 49.7 | NC0199794 | 3 | 146 | 0 | −0.07446 | 1.13 |
| A1ZM079744_AT | NC0013275 | 2 | 49.7 | NC0199913 | 3 | 152.8 | 0 | −0.05720 | 0.87 |
| A1ZM079744_AT | NC0013275 | 2 | 49.7 | NC0199750 | 6 | 81.6 | 0 | −0.06504 | 0.99 |
| A1ZM079744_AT | NC0013275 | 2 | 49.7 | NC0200165 | 6 | 99.9 | 0 | 0.05777 | 0.88 |
| A1ZM079744_AT | NC0013275 | 2 | 49.7 | NC0195223 | 6 | 118.6 | 0 | −0.07257 | 1.10 |
| A1ZM079744_AT | NC0013275 | 2 | 49.7 | NC0199409 | 6 | 118.6 | 0 | 0.06111 | 0.93 |
| A1ZM079744_AT | NC0013275 | 2 | 49.7 | NC0199958 | 6 | 142 | 0 | −0.05598 | 0.85 |
| A1ZM079744_AT | NC0013275 | 2 | 49.7 | NC0200185 | 7 | 147.7 | 0 | −0.07289 | 1.11 |
| A1ZM079744_AT | NC0013275 | 2 | 49.7 | NC0199331 | 7 | 173.1 | 0 | 0.06173 | 0.94 |
| A1ZM079744_AT | NC0013275 | 2 | 49.7 | NC0199896 | 8 | 96.4 | 0 | 0.05674 | 0.86 |
| A1ZM079744_AT | NC0013275 | 2 | 49.7 | NC0199810 | 10 | 48.3 | 0 | 0.05540 | 0.84 |
| A1ZM079744_AT | NC0013275 | 2 | 49.7 | NC0011002 | 10 | 66.5 | 0 | −0.06117 | 0.93 |
| A1ZM081073_AT | NC0109315 | 3 | 77.4 | NC0199760 | 1 | 179.1 | 0 | −0.14299 | 0.92 |
| A1ZM081073_AT | NC0109315 | 3 | 77.4 | NC0173731 | 2 | 138.4 | 0 | −0.13913 | 0.90 |
| A1ZM081073_AT | NC0109315 | 3 | 77.4 | NC0109315 | 3 | 77.4 | 1 | −0.17740 | 1.14 |
| A1ZM081073_AT | NC0109315 | 3 | 77.4 | NC0173641 | 3 | 80.9 | 0 | 0.16817 | 1.09 |
| A1ZM081073_AT | NC0109315 | 3 | 77.4 | NC0200132 | 4 | 128.7 | 0 | −0.14998 | 0.97 |
| A1ZM081073_AT | NC0109315 | 3 | 77.4 | NC0199754 | 7 | 77.6 | 0 | 0.16367 | 1.06 |
| A1ZM081073_AT | NC0109315 | 3 | 77.4 | NC0199722 | 9 | 79.6 | 0 | 0.13871 | 0.90 |
| A1ZM084532_AT | NC0173530 | 9 | 78.1 | NC0196949 | 2 | 43.4 | 0 | −0.09024 | 0.99 |
| A1ZM084532_AT | NC0173530 | 9 | 78.1 | NC0173731 | 2 | 138.4 | 0 | −0.09084 | 0.99 |
| A1ZM084532_AT | NC0173530 | 9 | 78.1 | NC0173436 | 3 | 86 | 0 | −0.08827 | 0.97 |
| A1ZM084532_AT | NC0173530 | 9 | 78.1 | NC0015965 | 3 | 140.8 | 0 | −0.08769 | 0.96 |
| A1ZM084532_AT | NC0173530 | 9 | 78.1 | NC0112924 | 3 | 145.3 | 0 | −0.08738 | 0.96 |
| A1ZM084532_AT | NC0173530 | 9 | 78.1 | NC0199794 | 3 | 146 | 0 | −0.08991 | 0.98 |
| A1ZM084532_AT | NC0173530 | 9 | 78.1 | NC0200132 | 4 | 128.7 | 0 | −0.08339 | 0.91 |
| A1ZM084532_AT | NC0173530 | 9 | 78.1 | NC0173476 | 7 | 44.2 | 0 | −0.08566 | 0.94 |
| A1ZM084532_AT | NC0173530 | 9 | 78.1 | NC0199896 | 8 | 96.4 | 0 | 0.10085 | 1.10 |
| A1ZM084532_AT | NC0173530 | 9 | 78.1 | NC0173350 | 8 | 104 | 0 | 0.08573 | 0.94 |
| A1ZM084532_AT | NC0173530 | 9 | 78.1 | NC0173530 | 9 | 78.1 | 1 | 0.10632 | 1.16 |
| A1ZM084532_AT | NC0173530 | 9 | 78.1 | NC0199722 | 9 | 79.6 | 0 | 0.09771 | 1.07 |
| A1ZM085633_AT | NC0199896 | 8 | 96.4 | NC0144205 | 1 | 38.5 | 0 | −0.07886 | 0.94 |
| A1ZM085633_AT | NC0199896 | 8 | 96.4 | NC0029053 | 1 | 88.1 | 0 | 0.08797 | 1.05 |
| A1ZM085633_AT | NC0199896 | 8 | 96.4 | NC0174066 | 3 | 168.4 | 0 | 0.08763 | 1.04 |
| A1ZM085633_AT | NC0199896 | 8 | 96.4 | NC0190159 | 3 | 168.4 | 0 | 0.08762 | 1.04 |
| A1ZM085633_AT | NC0199896 | 8 | 96.4 | NC0199442 | 5 | 22.4 | 0 | −0.07712 | 0.92 |
| A1ZM085633_AT | NC0199896 | 8 | 96.4 | NC0040322 | 7 | 64.5 | 0 | 0.08921 | 1.06 |
| A1ZM085633_AT | NC0199896 | 8 | 96.4 | NC0042930 | 7 | 64.5 | 0 | 0.07903 | 0.94 |
| A1ZM085633_AT | NC0199896 | 8 | 96.4 | NC0199312 | 7 | 65.9 | 0 | 0.08498 | 1.01 |
| A1ZM085633_AT | NC0199896 | 8 | 96.4 | NC0199313 | 8 | 89.3 | 0 | −0.07726 | 0.92 |
| A1ZM085633_AT | NC0199896 | 8 | 96.4 | NC0199896 | 8 | 96.4 | 1 | 0.10652 | 1.27 |
| A1ZM085633_AT | NC0199896 | 8 | 96.4 | NC0157736 | 9 | 56.6 | 0 | 0.08231 | 0.98 |
| A1ZM085633_AT | NC0199896 | 8 | 96.4 | NC0200122 | 9 | 56.6 | 0 | −0.08231 | 0.98 |
| A1ZM085633_AT | NC0199896 | 8 | 96.4 | NC0107256 | 9 | 68.5 | 0 | −0.07854 | 0.93 |
| A1ZM085633_AT | NC0199896 | 8 | 96.4 | NC0173530 | 9 | 78.1 | 0 | 0.07965 | 0.95 |
| A1ZM085633_AT | NC0199896 | 8 | 96.4 | NC0199722 | 9 | 79.6 | 0 | 0.07739 | 0.92 |
| A1ZM085633_AT | NC0199896 | 8 | 96.4 | NC0173818 | 10 | 65.3 | 0 | 0.08476 | 1.01 |

PLS Regression of Yield on Cis or Trans Oriented Proximal eQTL Markers

PLS regressions of yield on marker genotype were calculated separately for cis and trans orient markers. That is, one regression analysis was calculated for proximal eQTL markers having a “cis match” value equal to 1 in Table 5, and another regression analysis was calculated for proximal eQTL markers having a “cis match” value equal to 0.

Variation in cis oriented proximal eQTL marker genotypes accounted for only 38% of the variation in yield whereas the trans oriented counterparts accounted for 46% of yield variation, suggesting that variation in trans acting factors on gene expression at a locus is more important than variation at the locus itself (Table 6).

TABLE 6
PLS regression of yield on markers in cis orientation or in trans
orientation to probesets significantly associated with yield. Markers in both cis and trans
orientation are those where a proximal expression QTL marker is in cis orientation with
respect to one probeset and in trans orientation to another. Rsquare for the cis orientation
model = 38.34. Rsquare for the trans orientation model = 46.30.

| | | | | Proximal | Proximal |
|---|---|---|---|---|---|
| | | Proximal | Proximal | Expression | Expression |
| | Marker | Expression | Expression | QTL | QTL |
| proximal | Orientation | QTL | QTL | Marker | Marker |
| Expression QTL | To | Marker | Marker | Regression | Regression |
| Marker | Probesets | Chromosome | Position | Coefficient | VIP |
| NC0173332 | trans | 1 | 33.3 | −0.03384 | 1.08 |
| NC0199555 | cis | 1 | 34.4 | 0.07608 | 1.05 |
| NC0199555 | trans | 1 | 34.4 | 0.03465 | 1.10 |
| NC0036726 | trans | 1 | 35.2 | 0.02742 | 0.87 |
| NC0200044 | trans | 1 | 47.5 | −0.03512 | 1.12 |
| NC0199971 | trans | 1 | 66.1 | −0.02789 | 0.89 |
| NC0173453 | trans | 1 | 172.2 | −0.03340 | 1.06 |
| NC0199461 | cis | 1 | 176.7 | 0.06377 | 0.88 |
| NC0199461 | trans | 1 | 176.7 | 0.02904 | 0.92 |
| NC0199760 | trans | 1 | 179.1 | 0.02799 | 0.89 |
| NC0196949 | trans | 2 | 43.4 | 0.04416 | 1.41 |
| NC0013275 | cis | 2 | 49.7 | 0.08864 | 1.22 |
| NC0013275 | trans | 2 | 49.7 | 0.04037 | 1.28 |
| NC0199346 | cis | 2 | 50.8 | 0.07418 | 1.02 |
| NC0199346 | trans | 2 | 50.8 | 0.03379 | 1.08 |
| NC0002630 | trans | 2 | 54.1 | −0.02694 | 0.86 |
| NC0199297 | trans | 2 | 100.2 | −0.03168 | 1.01 |
| NC0051616 | trans | 3 | 40.4 | −0.02665 | 0.85 |
| NC0199803 | trans | 3 | 85.6 | 0.03135 | 1.00 |
| NC0173436 | cis | 3 | 86.0 | 0.09119 | 1.26 |
| NC0173436 | trans | 3 | 86.0 | 0.04153 | 1.32 |
| NC0000399 | trans | 3 | 121.1 | 0.03596 | 1.14 |
| NC0199741 | trans | 3 | 125.7 | 0.02536 | 0.81 |
| NC0015965 | cis | 3 | 140.8 | 0.07745 | 1.07 |
| NC0015965 | trans | 3 | 140.8 | 0.03527 | 1.12 |
| NC0112924 | cis | 3 | 145.3 | 0.06243 | 0.86 |
| NC0112924 | trans | 3 | 145.3 | 0.02844 | 0.91 |
| NC0199794 | trans | 3 | 146.0 | 0.04101 | 1.31 |
| NC0190032 | trans | 3 | 147.8 | 0.03191 | 1.02 |
| NC0199675 | trans | 5 | 45.9 | 0.02628 | 0.84 |
| NC0200120 | cis | 5 | 75.4 | −0.06401 | 0.88 |
| NC0200120 | trans | 5 | 75.4 | −0.02916 | 0.93 |
| NC0199447 | trans | 5 | 80.6 | 0.03496 | 1.11 |
| NC0173990 | trans | 5 | 98.9 | 0.02637 | 0.84 |
| NC0200212 | trans | 5 | 176.4 | −0.02603 | 0.83 |
| NC0173900 | trans | 6 | 38.4 | 0.03137 | 1.00 |
| NC0020696 | trans | 6 | 38.5 | −0.03057 | 0.97 |
| NC0173859 | trans | 7 | 96.8 | 0.02548 | 0.81 |
| NC0199889 | trans | 7 | 99.4 | 0.02550 | 0.81 |
| NC0200185 | trans | 7 | 147.7 | 0.02529 | 0.80 |
| NC0199339 | trans | 7 | 150.8 | −0.02758 | 0.88 |
| NC0187978 | trans | 7 | 165.7 | −0.02664 | 0.85 |
| NC0199331 | trans | 7 | 173.1 | −0.02928 | 0.93 |
| NC0000129 | trans | 8 | 16.5 | 0.02847 | 0.91 |
| NC0199443 | cis | 8 | 72.9 | 0.06037 | 0.83 |
| NC0199443 | trans | 8 | 72.9 | 0.02750 | 0.88 |
| NC0199730 | cis | 8 | 79.6 | 0.07407 | 1.02 |
| NC0199730 | trans | 8 | 79.6 | 0.03373 | 1.07 |
| NC0199735 | cis | 8 | 84.4 | 0.07071 | 0.97 |
| NC0199735 | trans | 8 | 84.4 | 0.03221 | 1.03 |
| NC0199770 | trans | 8 | 96.2 | 0.02575 | 0.82 |
| NC0199896 | cis | 8 | 96.4 | −0.07613 | 1.05 |
| NC0199896 | trans | 8 | 96.4 | −0.03467 | 1.10 |
| NC0033526 | cis | 8 | 153.1 | 0.08622 | 1.19 |
| NC0033526 | trans | 8 | 153.1 | 0.03927 | 1.25 |
| NC0014545 | trans | 8 | 155.1 | 0.03294 | 1.05 |
| NC0199956 | trans | 8 | 169.9 | −0.03128 | 1.00 |
| NC0173530 | cis | 9 | 78.1 | −0.05905 | 0.81 |
| NC0173530 | trans | 9 | 78.1 | −0.02690 | 0.86 |
| NC0199722 | cis | 9 | 79.6 | −0.06735 | 0.93 |
| NC0199722 | trans | 9 | 79.6 | −0.03067 | 0.98 |
| NC0028613 | trans | 9 | 94.6 | 0.02822 | 0.90 |
| NC0171018 | cis | 10 | 42.7 | 0.05886 | 0.81 |

Note that some markers proximal to a probeset were declared to be in both cis and trans orientation. This occurs because the marker in question is proximal to an eQTL in cis orientation to the particular probeset locus while at the same time being proximal to an eQTL in trans orientation to another probeset.

All markers, classified cis, trans, or both, were entered into another PLS regression analysis with yield (Table 7). In the final model, variation in marker genotype accounted for 47% of the variation of yield, exactly the same rsquare value obtained from the regression of yield on trans oriented eQTL markers only. This result reinforces the conclusion that variation in trans acting elements may be more important than variation at the structural genes themselves.

TABLE 7
PLS regression of yield on markers in either cis or trans orientation to probesets
significantly associated with yield. Markers in both cis and trans orientation are those
where a proximal expression QTL marker is in cis orientation with respect to one
probeset and in trans orientation to another. Rsquare for the regression model = 47.03.

| | | | | Proximal | Proximal |
|---|---|---|---|---|---|
| | | Proximal | Proximal | Expression | Expression |
| Proximal | | Expression | Expression | QTL | QTL |
| Expression | | QTL | QTL | Marker | Marker |
| QTL | Orientation To | Marker | Marker | Regression | Regression |
| Marker | Probesets | Chromosome | Position | Coefficient | VIP |
| NC0173332 | trans | 1 | 33.3 | −0.03257 | 1.09 |
| NC0199555 | cis & trans | 1 | 34.4 | 0.03335 | 1.12 |
| NC0200044 | trans | 1 | 47.5 | −0.03379 | 1.13 |
| NC0143873 | trans | 1 | 52.6 | −0.02388 | 0.80 |
| NC0199971 | trans | 1 | 66.1 | −0.02684 | 0.90 |
| NC0173453 | trans | 1 | 172.2 | −0.03214 | 1.08 |
| NC0199461 | cis & trans | 1 | 176.7 | 0.02795 | 0.94 |
| NC0199760 | trans | 1 | 179.1 | 0.02693 | 0.90 |
| NC0196949 | trans | 2 | 43.4 | 0.04250 | 1.43 |
| NC0013275 | cis & trans | 2 | 49.7 | 0.03885 | 1.30 |
| NC0199346 | cis & trans | 2 | 50.8 | 0.03252 | 1.09 |
| NC0002630 | trans | 2 | 54.1 | −0.02593 | 0.87 |
| NC0199297 | trans | 2 | 100.2 | −0.03049 | 1.02 |
| NC0051616 | trans | 3 | 40.4 | −0.02564 | 0.86 |
| NC0199803 | trans | 3 | 85.6 | 0.03017 | 1.01 |
| NC0173436 | cis & trans | 3 | 86.0 | 0.03997 | 1.34 |
| NC0199676 | trans | 3 | 91.5 | −0.02390 | 0.80 |
| NC0000399 | trans | 3 | 121.1 | 0.03461 | 1.16 |
| NC0199741 | cis & trans | 3 | 125.7 | 0.02441 | 0.82 |
| NC0015965 | cis & trans | 3 | 140.8 | 0.03395 | 1.14 |
| NC0112924 | cis & trans | 3 | 145.3 | 0.02737 | 0.92 |
| NC0199794 | trans | 3 | 146.0 | 0.03947 | 1.32 |
| NC0190032 | trans | 3 | 147.8 | 0.03071 | 1.03 |
| NC0199675 | trans | 5 | 45.9 | 0.02529 | 0.85 |
| NC0200120 | cis & trans | 5 | 75.4 | −0.02806 | 0.94 |
| NC0199447 | trans | 5 | 80.6 | 0.03364 | 1.13 |
| NC0173990 | trans | 5 | 98.9 | 0.02538 | 0.85 |
| NC0200212 | trans | 5 | 176.4 | −0.02505 | 0.84 |
| NC0173900 | trans | 6 | 38.4 | 0.03019 | 1.01 |
| NC0020696 | trans | 6 | 38.5 | −0.02942 | 0.99 |
| NC0173859 | cis & trans | 7 | 96.8 | 0.02452 | 0.82 |
| NC0199889 | trans | 7 | 99.4 | 0.02454 | 0.82 |
| NC0200185 | cis & trans | 7 | 147.7 | 0.02434 | 0.82 |
| NC0199339 | trans | 7 | 150.8 | −0.02655 | 0.89 |
| NC0187978 | trans | 7 | 165.7 | −0.02564 | 0.86 |
| NC0199331 | trans | 7 | 173.1 | −0.02818 | 0.94 |
| NC0000129 | trans | 8 | 16.5 | 0.02739 | 0.92 |
| NC0199443 | cis & trans | 8 | 72.9 | 0.02646 | 0.89 |
| NC0199730 | cis & trans | 8 | 79.6 | 0.03247 | 1.09 |
| NC0199735 | cis & trans | 8 | 84.4 | 0.03100 | 1.04 |
| NC0199770 | trans | 8 | 96.2 | 0.02478 | 0.83 |
| NC0199896 | cis & trans | 8 | 96.4 | −0.03337 | 1.12 |
| NC0033526 | cis & trans | 8 | 153.1 | 0.03779 | 1.27 |
| NC0014545 | trans | 8 | 155.1 | 0.03170 | 1.06 |
| NC0199956 | trans | 8 | 169.9 | −0.03010 | 1.01 |
| NC0020872 | trans | 9 | 76.3 | 0.02398 | 0.80 |
| NC0173530 | cis & trans | 9 | 78.1 | −0.02588 | 0.87 |
| NC0199722 | cis & trans | 9 | 79.6 | −0.02952 | 0.99 |
| NC0028613 | trans | 9 | 94.6 | 0.02716 | 0.91 |
| NC0171018 | cis | 10 | 42.7 | 0.02580 | 0.87 |

The cis-trans orientation of all proximal eQTL markers to probesets is more fully portrayed in Table 8.

TABLE 8
Cis-trans orientation of proximal expression eQTL markers relative to probeset position.

| | | | | | | | | Proximal |
|---|---|---|---|---|---|---|---|---|
| | | | | | | Proximal | | Expression |
| | | Proximal | Proximal | Proximal | Proximal | Expression | Probeset | QTL |
| | Proximal | Probeset | Probset | Expression | Expression | QTL | To | Marker |
| | Probeset | Marker | Marker | QTL | QTL Marker | Marker | Marker | Regression |
| Probeset | Marker | Chromosome | Position | Marker | Chromosome | Position | Probeset | Position |
| A1ZM005469_AT | NC0070876 | 1 | 19.7 | NC0173332 | 1 | 33.3 | trans | −0.03429 |
| A1ZM005469_AT | NC0070876 | 1 | 19.7 | NC0187978 | 7 | 165.7 | trans | −0.02700 |
| A1ZM005469_AT | NC0070876 | 1 | 19.7 | NC0199331 | 7 | 173.1 | trans | −0.02967 |
| A1ZM005469_AT | NC0070876 | 1 | 19.7 | NC0199735 | 8 | 84.4 | trans | 0.03264 |
| A1ZM005469_AT | NC0070876 | 1 | 19.7 | NC0199896 | 8 | 96.4 | trans | −0.03513 |
| A1ZM052424_S_AT | NC0173400 | 1 | 32.2 | NC0173900 | 6 | 38.4 | trans | 0.03178 |
| A1ZM052424_S_AT | NC0173400 | 1 | 32.2 | NC0200120 | 5 | 75.4 | trans | −0.02954 |
| A1ZM061580_AT | NC0199555 | 1 | 34.4 | NC0036726 | 1 | 35.2 | trans | 0.02779 |
| A1ZM061580_AT | NC0199555 | 1 | 34.4 | NC0173332 | 1 | 33.3 | trans | −0.03429 |
| A1ZM061580_AT | NC0199555 | 1 | 34.4 | NC0199555 | 1 | 34.4 | cis | 0.03511 |
| A1ZM021664_AT | NC0200213 | 1 | 67.4 | NC0173332 | 1 | 33.3 | trans | −0.03429 |
| A1ZM021664_AT | NC0200213 | 1 | 67.4 | NC0173530 | 9 | 78.1 | trans | −0.02725 |
| A1ZM021664_AT | NC0200213 | 1 | 67.4 | NC0173859 | 7 | 96.8 | trans | 0.02582 |
| A1ZM021664_AT | NC0200213 | 1 | 67.4 | NC0187978 | 7 | 165.7 | trans | −0.02700 |
| A1ZM021664_AT | NC0200213 | 1 | 67.4 | NC0199331 | 7 | 173.1 | trans | −0.02967 |
| A1ZM021664_AT | NC0200213 | 1 | 67.4 | NC0199346 | 2 | 50.8 | trans | 0.03424 |
| A1ZM021664_AT | NC0200213 | 1 | 67.4 | NC0199555 | 1 | 34.4 | trans | 0.03511 |
| A1ZM021664_AT | NC0200213 | 1 | 67.4 | NC0199971 | 1 | 66.1 | trans | −0.02826 |
| A1ZM021664_AT | NC0200213 | 1 | 67.4 | NC0200044 | 1 | 47.5 | trans | −0.03558 |
| A1ZM021664_AT | NC0200213 | 1 | 67.4 | NC0200185 | 7 | 147.7 | trans | 0.02563 |
| A1ZM002154_S_AT | NC0199461 | 1 | 176.7 | NC0014545 | 8 | 155.1 | trans | 0.03338 |
| A1ZM002154_S_AT | NC0199461 | 1 | 176.7 | NC0033526 | 8 | 153.1 | trans | 0.03979 |
| A1ZM002154_S_AT | NC0199461 | 1 | 176.7 | NC0051616 | 3 | 40.4 | trans | −0.02700 |
| A1ZM002154_S_AT | NC0199461 | 1 | 176.7 | NC0199297 | 2 | 100.2 | trans | −0.03210 |
| A1ZM002154_S_AT | NC0199461 | 1 | 176.7 | NC0199443 | 8 | 72.9 | trans | 0.02786 |
| A1ZM002154_S_AT | NC0199461 | 1 | 176.7 | NC0199461 | 1 | 176.7 | cis | 0.02943 |
| A1ZM002154_S_AT | NC0199461 | 1 | 176.7 | NC0199730 | 8 | 79.6 | trans | 0.03418 |
| A1ZM002154_S_AT | NC0199461 | 1 | 176.7 | NC0199735 | 8 | 84.4 | trans | 0.03264 |
| A1ZM069545_AT | NC0199461 | 1 | 176.7 | NC0000399 | 3 | 121.1 | trans | 0.03644 |
| A1ZM069545_AT | NC0199461 | 1 | 176.7 | NC0013275 | 2 | 49.7 | trans | 0.04091 |
| A1ZM069545_AT | NC0199461 | 1 | 176.7 | NC0014545 | 8 | 155.1 | trans | 0.03338 |
| A1ZM069545_AT | NC0199461 | 1 | 176.7 | NC0015965 | 3 | 140.8 | trans | 0.03574 |
| A1ZM069545_AT | NC0199461 | 1 | 176.7 | NC0028613 | 9 | 94.6 | trans | 0.02860 |
| A1ZM069545_AT | NC0199461 | 1 | 176.7 | NC0033526 | 8 | 153.1 | trans | 0.03979 |
| A1ZM069545_AT | NC0199461 | 1 | 176.7 | NC0112924 | 3 | 145.3 | trans | 0.02881 |
| A1ZM069545_AT | NC0199461 | 1 | 176.7 | NC0199297 | 2 | 100.2 | trans | −0.03210 |
| A1ZM069545_AT | NC0199461 | 1 | 176.7 | NC0199443 | 8 | 72.9 | trans | 0.02786 |
| A1ZM069545_AT | NC0199461 | 1 | 176.7 | NC0199461 | 1 | 176.7 | cis | 0.02943 |
| A1ZM069545_AT | NC0199461 | 1 | 176.7 | NC0199730 | 8 | 79.6 | trans | 0.03418 |
| A1ZM069545_AT | NC0199461 | 1 | 176.7 | NC0199794 | 3 | 146 | trans | 0.04156 |
| A1ZM000016_S_AT | NC0173951 | 1 | 229.4 | NC0000129 | 8 | 16.5 | trans | 0.02884 |
| A1ZM000016_S_AT | NC0173951 | 1 | 229.4 | NC0173900 | 6 | 38.4 | trans | 0.03178 |
| A1ZM000016_S_AT | NC0173951 | 1 | 229.4 | NC0199331 | 7 | 173.1 | trans | −0.02967 |
| A1ZM000016_S_AT | NC0173951 | 1 | 229.4 | NC0199722 | 9 | 79.6 | trans | −0.03108 |
| A1ZM000016_S_AT | NC0173951 | 1 | 229.4 | NC0199760 | 1 | 179.1 | trans | 0.02836 |
| A1ZM020483_A_AT | NC0173951 | 1 | 229.4 | NC0000129 | 8 | 16.5 | trans | 0.02884 |
| A1ZM020483_A_AT | NC0173951 | 1 | 229.4 | NC0173859 | 7 | 96.8 | trans | 0.02582 |
| A1ZM020483_A_AT | NC0173951 | 1 | 229.4 | NC0199889 | 7 | 99.4 | trans | 0.02584 |
| A1ZM020483_A_AT | NC0173951 | 1 | 229.4 | NC0200185 | 7 | 147.7 | trans | 0.02563 |
| A1ZM063309_AT | NC0173951 | 1 | 229.4 | NC0013275 | 2 | 49.7 | trans | 0.04091 |
| A1ZM063309_AT | NC0173951 | 1 | 229.4 | NC0199346 | 2 | 50.8 | trans | 0.03424 |
| A1ZM063309_AT | NC0173951 | 1 | 229.4 | NC0200185 | 7 | 147.7 | trans | 0.02563 |
| A1ZM003299_S_AT | NC0013275 | 2 | 49.7 | NC0002630 | 2 | 54.1 | trans | −0.02730 |
| A1ZM003299_S_AT | NC0013275 | 2 | 49.7 | NC0013275 | 2 | 49.7 | cis | 0.04091 |
| A1ZM003299_S_AT | NC0013275 | 2 | 49.7 | NC0015965 | 3 | 140.8 | trans | 0.03574 |
| A1ZM003299_S_AT | NC0013275 | 2 | 49.7 | NC0196949 | 2 | 43.4 | trans | 0.04475 |
| A1ZM003299_S_AT | NC0013275 | 2 | 49.7 | NC0199346 | 2 | 50.8 | trans | 0.03424 |
| A1ZM003299_S_AT | NC0013275 | 2 | 49.7 | NC0199461 | 1 | 176.7 | trans | 0.02943 |
| A1ZM003299_S_AT | NC0013275 | 2 | 49.7 | NC0199722 | 9 | 79.6 | trans | −0.03108 |
| A1ZM003299_S_AT | NC0013275 | 2 | 49.7 | NC0199770 | 8 | 96.2 | trans | 0.02609 |
| A1ZM003299_S_AT | NC0013275 | 2 | 49.7 | NC0199794 | 3 | 146 | trans | 0.04156 |
| A1ZM061606_X_AT | NC0013275 | 2 | 49.7 | NC0013275 | 2 | 49.7 | cis | 0.04091 |
| A1ZM061606_X_AT | NC0013275 | 2 | 49.7 | NC0015965 | 3 | 140.8 | trans | 0.03574 |
| A1ZM061606_X_AT | NC0013275 | 2 | 49.7 | NC0199346 | 2 | 50.8 | trans | 0.03424 |
| A1ZM061606_X_AT | NC0013275 | 2 | 49.7 | NC0199461 | 1 | 176.7 | trans | 0.02943 |
| A1ZM061606_X_AT | NC0013275 | 2 | 49.7 | NC0199722 | 9 | 79.6 | trans | −0.03108 |
| A1ZM061606_X_AT | NC0013275 | 2 | 49.7 | NC0199735 | 8 | 84.4 | trans | 0.03264 |
| A1ZM061606_X_AT | NC0013275 | 2 | 49.7 | NC0199794 | 3 | 146 | trans | 0.04156 |
| A1ZM067500_AT | NC0013275 | 2 | 49.7 | NC0013275 | 2 | 49.7 | cis | 0.04091 |
| A1ZM067500_AT | NC0013275 | 2 | 49.7 | NC0173436 | 3 | 86 | trans | 0.04209 |
| A1ZM067500_AT | NC0013275 | 2 | 49.7 | NC0196949 | 2 | 43.4 | trans | 0.04475 |
| A1ZM067500_AT | NC0013275 | 2 | 49.7 | NC0199346 | 2 | 50.8 | trans | 0.03424 |
| A1ZM067500_AT | NC0013275 | 2 | 49.7 | NC0199722 | 9 | 79.6 | trans | −0.03108 |
| A1ZM067500_AT | NC0013275 | 2 | 49.7 | NC0199794 | 3 | 146 | trans | 0.04156 |
| A1ZM079744_AT | NC0013275 | 2 | 49.7 | NC0013275 | 2 | 49.7 | cis | 0.04091 |
| A1ZM079744_AT | NC0013275 | 2 | 49.7 | NC0015965 | 3 | 140.8 | trans | 0.03574 |
| A1ZM079744_AT | NC0013275 | 2 | 49.7 | NC0112924 | 3 | 145.3 | trans | 0.02881 |
| A1ZM079744_AT | NC0013275 | 2 | 49.7 | NC0196949 | 2 | 43.4 | trans | 0.04475 |
| A1ZM079744_AT | NC0013275 | 2 | 49.7 | NC0199331 | 7 | 173.1 | trans | −0.02967 |
| A1ZM079744_AT | NC0013275 | 2 | 49.7 | NC0199346 | 2 | 50.8 | trans | 0.03424 |
| A1ZM079744_AT | NC0013275 | 2 | 49.7 | NC0199741 | 3 | 125.7 | trans | 0.02570 |
| A1ZM079744_AT | NC0013275 | 2 | 49.7 | NC0199794 | 3 | 146 | trans | 0.04156 |
| A1ZM079744_AT | NC0013275 | 2 | 49.7 | NC0199896 | 8 | 96.4 | trans | −0.03513 |
| A1ZM079744_AT | NC0013275 | 2 | 49.7 | NC0200185 | 7 | 147.7 | trans | 0.02563 |
| A1ZM025769_AT | NC0199346 | 2 | 50.8 | NC0002630 | 2 | 54.1 | trans | −0.02730 |
| A1ZM025769_AT | NC0199346 | 2 | 50.8 | NC0013275 | 2 | 49.7 | trans | 0.04091 |
| A1ZM025769_AT | NC0199346 | 2 | 50.8 | NC0199331 | 7 | 173.1 | trans | −0.02967 |
| A1ZM025769_AT | NC0199346 | 2 | 50.8 | NC0199346 | 2 | 50.8 | cis | 0.03424 |
| A1ZM025769_AT | NC0199346 | 2 | 50.8 | NC0199447 | 5 | 80.6 | trans | 0.03542 |
| A1ZM025769_AT | NC0199346 | 2 | 50.8 | NC0199794 | 3 | 146 | trans | 0.04156 |
| A1ZM025769_AT | NC0199346 | 2 | 50.8 | NC0200120 | 5 | 75.4 | trans | −0.02954 |
| A1ZM044683_X_AT | NC0174062 | 2 | 129.5 | NC0173453 | 1 | 172.2 | trans | −0.03384 |
| A1ZM039148_AT | NC0003536 | 3 | 30.9 | NC0015965 | 3 | 140.8 | trans | 0.03574 |
| A1ZM039148_AT | NC0003536 | 3 | 30.9 | NC0190032 | 3 | 147.8 | trans | 0.03234 |
| A1ZM081073_AT | NC0109315 | 3 | 77.4 | NC0199722 | 9 | 79.6 | trans | −0.03108 |
| A1ZM081073_AT | NC0109315 | 3 | 77.4 | NC0199760 | 1 | 179.1 | trans | 0.02836 |
| A1ZM025661_AT | NC0173436 | 3 | 86 | NC0173436 | 3 | 86 | cis | 0.04209 |
| A1ZM025661_AT | NC0173436 | 3 | 86 | NC0187978 | 7 | 165.7 | trans | −0.02700 |
| A1ZM025661_AT | NC0173436 | 3 | 86 | NC0199730 | 8 | 79.6 | trans | 0.03418 |
| A1ZM025661_AT | NC0173436 | 3 | 86 | NC0199803 | 3 | 85.6 | trans | 0.03177 |
| A1ZM025661_AT | NC0173436 | 3 | 86 | NC0200044 | 1 | 47.5 | trans | −0.03558 |
| A1ZM025661_AT | NC0173436 | 3 | 86 | NC0200212 | 5 | 176.4 | trans | −0.02637 |
| A1ZM030937_AT | NC0173436 | 3 | 86 | NC0015965 | 3 | 140.8 | trans | 0.03574 |
| A1ZM030937_AT | NC0173436 | 3 | 86 | NC0173436 | 3 | 86 | cis | 0.04209 |
| A1ZM030937_AT | NC0173436 | 3 | 86 | NC0173530 | 9 | 78.1 | trans | −0.02725 |
| A1ZM030937_AT | NC0173436 | 3 | 86 | NC0199346 | 2 | 50.8 | trans | 0.03424 |
| A1ZM030937_AT | NC0173436 | 3 | 86 | NC0199722 | 9 | 79.6 | trans | −0.03108 |
| A1ZM030937_AT | NC0173436 | 3 | 86 | NC0199794 | 3 | 146 | trans | 0.04156 |
| A1ZM030937_AT | NC0173436 | 3 | 86 | NC0199803 | 3 | 85.6 | trans | 0.03177 |
| A1ZM058469_AT | NC0173436 | 3 | 86 | NC0173436 | 3 | 86 | cis | 0.04209 |
| A1ZM058469_AT | NC0173436 | 3 | 86 | NC0199803 | 3 | 85.6 | trans | 0.03177 |
| A1ZM056031_AT | NC0199589 | 3 | 91.5 | NC0015965 | 3 | 140.8 | trans | 0.03574 |
| A1ZM056031_AT | NC0199589 | 3 | 91.5 | NC0173436 | 3 | 86 | trans | 0.04209 |
| A1ZM056031_AT | NC0199589 | 3 | 91.5 | NC0190032 | 3 | 147.8 | trans | 0.03234 |
| A1ZM056031_AT | NC0199589 | 3 | 91.5 | NC0199794 | 3 | 146 | trans | 0.04156 |
| A1ZM011510_S_AT | NC0199741 | 3 | 125.7 | NC0000399 | 3 | 121.1 | trans | 0.03644 |
| A1ZM011510_S_AT | NC0199741 | 3 | 125.7 | NC0013275 | 2 | 49.7 | trans | 0.04091 |
| A1ZM011510_S_AT | NC0199741 | 3 | 125.7 | NC0036726 | 1 | 35.2 | trans | 0.02779 |
| A1ZM011510_S_AT | NC0199741 | 3 | 125.7 | NC0112924 | 3 | 145.3 | trans | 0.02881 |
| A1ZM011510_S_AT | NC0199741 | 3 | 125.7 | NC0173436 | 3 | 86 | trans | 0.04209 |
| A1ZM011510_S_AT | NC0199741 | 3 | 125.7 | NC0173530 | 9 | 78.1 | trans | −0.02725 |
| A1ZM011510_S_AT | NC0199741 | 3 | 125.7 | NC0196949 | 2 | 43.4 | trans | 0.04475 |
| A1ZM011510_S_AT | NC0199741 | 3 | 125.7 | NC0199722 | 9 | 79.6 | trans | −0.03108 |
| A1ZM011510_S_AT | NC0199741 | 3 | 125.7 | NC0199730 | 8 | 79.6 | trans | 0.03418 |
| A1ZM011510_S_AT | NC0199741 | 3 | 125.7 | NC0199741 | 3 | 125.7 | cis | 0.02570 |
| A1ZM011510_S_AT | NC0199741 | 3 | 125.7 | NC0199770 | 8 | 96.2 | trans | 0.02609 |
| A1ZM011510_S_AT | NC0199741 | 3 | 125.7 | NC0199794 | 3 | 146 | trans | 0.04156 |
| A1ZM011510_S_AT | NC0199741 | 3 | 125.7 | NC0199896 | 8 | 96.4 | trans | −0.03513 |
| A1ZM006707_AT | NC0112924 | 3 | 145.3 | NC0000129 | 8 | 16.5 | trans | 0.02884 |
| A1ZM006707_AT | NC0112924 | 3 | 145.3 | NC0002630 | 2 | 54.1 | trans | −0.02730 |
| A1ZM006707_AT | NC0112924 | 3 | 145.3 | NC0013275 | 2 | 49.7 | trans | 0.04091 |
| A1ZM006707_AT | NC0112924 | 3 | 145.3 | NC0112924 | 3 | 145.3 | cis | 0.02881 |
| A1ZM006707_AT | NC0112924 | 3 | 145.3 | NC0199346 | 2 | 50.8 | trans | 0.03424 |
| A1ZM006707_AT | NC0112924 | 3 | 145.3 | NC0199461 | 1 | 176.7 | trans | 0.02943 |
| A1ZM006707_AT | NC0112924 | 3 | 145.3 | NC0199722 | 9 | 79.6 | trans | −0.03108 |
| A1ZM006707_AT | NC0112924 | 3 | 145.3 | NC0199735 | 8 | 84.4 | trans | 0.03264 |
| A1ZM006707_AT | NC0112924 | 3 | 145.3 | NC0199770 | 8 | 96.2 | trans | 0.02609 |
| A1ZM006707_AT | NC0112924 | 3 | 145.3 | NC0199803 | 3 | 85.6 | trans | 0.03177 |
| A1ZM077940_X_AT | NC0193457 | 3 | 216.6 | NC0013275 | 2 | 49.7 | trans | 0.04091 |
| A1ZM077940_X_AT | NC0193457 | 3 | 216.6 | NC0173436 | 3 | 86 | trans | 0.04209 |
| A1ZM033184_AT | NC0200093 | 5 | 131.8 | NC0013275 | 2 | 49.7 | trans | 0.04091 |
| A1ZM033184_AT | NC0200093 | 5 | 131.8 | NC0015965 | 3 | 140.8 | trans | 0.03574 |
| A1ZM033184_AT | NC0200093 | 5 | 131.8 | NC0173530 | 9 | 78.1 | trans | −0.02725 |
| A1ZM033184_AT | NC0200093 | 5 | 131.8 | NC0199346 | 2 | 50.8 | trans | 0.03424 |
| A1ZM033184_AT | NC0200093 | 5 | 131.8 | NC0199722 | 9 | 79.6 | trans | −0.03108 |
| A1ZM033184_AT | NC0200093 | 5 | 131.8 | NC0200185 | 7 | 147.7 | trans | 0.02563 |
| A1ZM057468_AT | NC0036529 | 6 | 43.2 | NC0013275 | 2 | 49.7 | trans | 0.04091 |
| A1ZM057468_AT | NC0036529 | 6 | 43.2 | NC0015965 | 3 | 140.8 | trans | 0.03574 |
| A1ZM057468_AT | NC0036529 | 6 | 43.2 | NC0173530 | 9 | 78.1 | trans | −0.02725 |
| A1ZM057468_AT | NC0036529 | 6 | 43.2 | NC0190032 | 3 | 147.8 | trans | 0.03234 |
| A1ZM057468_AT | NC0036529 | 6 | 43.2 | NC0199722 | 9 | 79.6 | trans | −0.03108 |
| A1ZM057468_AT | NC0036529 | 6 | 43.2 | NC0199760 | 1 | 179.1 | trans | 0.02836 |
| A1ZM057468_AT | NC0036529 | 6 | 43.2 | NC0199794 | 3 | 146 | trans | 0.04156 |
| A1ZM057468_AT | NC0036529 | 6 | 43.2 | NC0199889 | 7 | 99.4 | trans | 0.02584 |
| A1ZM023477_S_AT | NC0028658 | 6 | 52.7 | NC0013275 | 2 | 49.7 | trans | 0.04091 |
| A1ZM023477_S_AT | NC0028658 | 6 | 52.7 | NC0199346 | 2 | 50.8 | trans | 0.03424 |
| A1ZM062662_AT | NC0195223 | 6 | 118.6 | NC0173436 | 3 | 86 | trans | 0.04209 |
| A1ZM062662_AT | NC0195223 | 6 | 118.6 | NC0199722 | 9 | 79.6 | trans | −0.03108 |
| A1ZM062662_AT | NC0195223 | 6 | 118.6 | NC0199741 | 3 | 125.7 | trans | 0.02570 |
| A1ZM062662_AT | NC0195223 | 6 | 118.6 | NC0200185 | 7 | 147.7 | trans | 0.02563 |
| A1ZM057238_AT | NC0199958 | 6 | 142 | NC0000129 | 8 | 16.5 | trans | 0.02884 |
| A1ZM057238_AT | NC0199958 | 6 | 142 | NC0002630 | 2 | 54.1 | trans | −0.02730 |
| A1ZM057238_AT | NC0199958 | 6 | 142 | NC0015965 | 3 | 140.8 | trans | 0.03574 |
| A1ZM057238_AT | NC0199958 | 6 | 142 | NC0028613 | 9 | 94.6 | trans | 0.02860 |
| A1ZM057238_AT | NC0199958 | 6 | 142 | NC0112924 | 3 | 145.3 | trans | 0.02881 |
| A1ZM057238_AT | NC0199958 | 6 | 142 | NC0173530 | 9 | 78.1 | trans | −0.02725 |
| A1ZM057238_AT | NC0199958 | 6 | 142 | NC0199346 | 2 | 50.8 | trans | 0.03424 |
| A1ZM057238_AT | NC0199958 | 6 | 142 | NC0199722 | 9 | 79.6 | trans | −0.03108 |
| A1ZM057238_AT | NC0199958 | 6 | 142 | NC0199794 | 3 | 146 | trans | 0.04156 |
| A1ZM057238_AT | NC0199958 | 6 | 142 | NC0200044 | 1 | 47.5 | trans | −0.03558 |
| A1ZM057856_AT | NC0173859 | 7 | 96.8 | NC0173436 | 3 | 86 | trans | 0.04209 |
| A1ZM057856_AT | NC0173859 | 7 | 96.8 | NC0173859 | 7 | 96.8 | cis | 0.02582 |
| A1ZM057856_AT | NC0173859 | 7 | 96.8 | NC0199339 | 7 | 150.8 | trans | −0.02795 |
| A1ZM057856_AT | NC0173859 | 7 | 96.8 | NC0199889 | 7 | 99.4 | trans | 0.02584 |
| A1ZM057856_AT | NC0173859 | 7 | 96.8 | NC0200044 | 1 | 47.5 | trans | −0.03558 |
| A1ZM026886_AT | NC0038733 | 7 | 106.5 | NC0173530 | 9 | 78.1 | trans | −0.02725 |
| A1ZM026886_AT | NC0038733 | 7 | 106.5 | NC0199722 | 9 | 79.6 | trans | −0.03108 |
| A1ZM045717_AT | NC0200185 | 7 | 147.7 | NC0013275 | 2 | 49.7 | trans | 0.04091 |
| A1ZM045717_AT | NC0200185 | 7 | 147.7 | NC0015965 | 3 | 140.8 | trans | 0.03574 |
| A1ZM045717_AT | NC0200185 | 7 | 147.7 | NC0173530 | 9 | 78.1 | trans | −0.02725 |
| A1ZM045717_AT | NC0200185 | 7 | 147.7 | NC0200185 | 7 | 147.7 | cis | 0.02563 |
| A1ZM006628_A_AT | NC0199443 | 8 | 72.9 | NC0020696 | 6 | 38.5 | trans | −0.03098 |
| A1ZM006628_A_AT | NC0199443 | 8 | 72.9 | NC0033526 | 8 | 153.1 | trans | 0.03979 |
| A1ZM006628_A_AT | NC0199443 | 8 | 72.9 | NC0112924 | 3 | 145.3 | trans | 0.02881 |
| A1ZM006628_A_AT | NC0199443 | 8 | 72.9 | NC0199331 | 7 | 173.1 | trans | −0.02967 |
| A1ZM006628_A_AT | NC0199443 | 8 | 72.9 | NC0199443 | 8 | 72.9 | cis | 0.02786 |
| A1ZM006628_A_AT | NC0199443 | 8 | 72.9 | NC0199730 | 8 | 79.6 | trans | 0.03418 |
| A1ZM006628_A_AT | NC0199443 | 8 | 72.9 | NC0199735 | 8 | 84.4 | trans | 0.03264 |
| A1ZM006628_A_AT | NC0199443 | 8 | 72.9 | NC0199770 | 8 | 96.2 | trans | 0.02609 |
| A1ZM006628_A_AT | NC0199443 | 8 | 72.9 | NC0199896 | 8 | 96.4 | trans | −0.03513 |
| A1ZM006628_A_AT | NC0199443 | 8 | 72.9 | NC0199956 | 8 | 169.9 | trans | −0.03169 |
| A1ZM025982_AT | NC0199730 | 8 | 79.6 | NC0199730 | 8 | 79.6 | cis | 0.03418 |
| A1ZM025982_AT | NC0199730 | 8 | 79.6 | NC0199735 | 8 | 84.4 | trans | 0.03264 |
| A1ZM025982_AT | NC0199730 | 8 | 79.6 | NC0199770 | 8 | 96.2 | trans | 0.02609 |
| A1ZM025982_AT | NC0199730 | 8 | 79.6 | NC0199896 | 8 | 96.4 | trans | −0.03513 |
| A1ZM015898_AT | NC0199735 | 8 | 84.4 | NC0013275 | 2 | 49.7 | trans | 0.04091 |
| A1ZM015898_AT | NC0199735 | 8 | 84.4 | NC0199339 | 7 | 150.8 | trans | −0.02795 |
| A1ZM015898_AT | NC0199735 | 8 | 84.4 | NC0199346 | 2 | 50.8 | trans | 0.03424 |
| A1ZM015898_AT | NC0199735 | 8 | 84.4 | NC0199730 | 8 | 79.6 | trans | 0.03418 |
| A1ZM015898_AT | NC0199735 | 8 | 84.4 | NC0199735 | 8 | 84.4 | cis | 0.03264 |
| A1ZM015898_AT | NC0199735 | 8 | 84.4 | NC0199770 | 8 | 96.2 | trans | 0.02609 |
| A1ZM015898_AT | NC0199735 | 8 | 84.4 | NC0199896 | 8 | 96.4 | trans | −0.03513 |
| A1ZM019225_AT | NC0199896 | 8 | 96.4 | NC0013275 | 2 | 49.7 | trans | 0.04091 |
| A1ZM019225_AT | NC0199896 | 8 | 96.4 | NC0112924 | 3 | 145.3 | trans | 0.02881 |
| A1ZM019225_AT | NC0199896 | 8 | 96.4 | NC0196949 | 2 | 43.4 | trans | 0.04475 |
| A1ZM019225_AT | NC0199896 | 8 | 96.4 | NC0199331 | 7 | 173.1 | trans | −0.02967 |
| A1ZM019225_AT | NC0199896 | 8 | 96.4 | NC0199443 | 8 | 72.9 | trans | 0.02786 |
| A1ZM019225_AT | NC0199896 | 8 | 96.4 | NC0199730 | 8 | 79.6 | trans | 0.03418 |
| A1ZM019225_AT | NC0199896 | 8 | 96.4 | NC0199735 | 8 | 84.4 | trans | 0.03264 |
| A1ZM019225_AT | NC0199896 | 8 | 96.4 | NC0199760 | 1 | 179.1 | trans | 0.02836 |
| A1ZM019225_AT | NC0199896 | 8 | 96.4 | NC0199770 | 8 | 96.2 | trans | 0.02609 |
| A1ZM019225_AT | NC0199896 | 8 | 96.4 | NC0199794 | 3 | 146 | trans | 0.04156 |
| A1ZM019225_AT | NC0199896 | 8 | 96.4 | NC0199896 | 8 | 96.4 | cis | −0.03513 |
| A1ZM019225_AT | NC0199896 | 8 | 96.4 | NC0199956 | 8 | 169.9 | trans | −0.03169 |
| A1ZM019225_AT | NC0199896 | 8 | 96.4 | NC0200185 | 7 | 147.7 | trans | 0.02563 |
| A1ZM085633_AT | NC0199896 | 8 | 96.4 | NC0173530 | 9 | 78.1 | trans | −0.02725 |
| A1ZM085633_AT | NC0199896 | 8 | 96.4 | NC0199722 | 9 | 79.6 | trans | −0.03108 |
| A1ZM085633_AT | NC0199896 | 8 | 96.4 | NC0199896 | 8 | 96.4 | cis | −0.03513 |
| A1ZM043652_S_AT | NC0173626 | 8 | 96.8 | NC0013275 | 2 | 49.7 | trans | 0.04091 |
| A1ZM043652_S_AT | NC0173626 | 8 | 96.8 | NC0173436 | 3 | 86 | trans | 0.04209 |
| A1ZM043652_S_AT | NC0173626 | 8 | 96.8 | NC0199461 | 1 | 176.7 | trans | 0.02943 |
| A1ZM043652_S_AT | NC0173626 | 8 | 96.8 | NC0199730 | 8 | 79.6 | trans | 0.03418 |
| A1ZM043652_S_AT | NC0173626 | 8 | 96.8 | NC0199735 | 8 | 84.4 | trans | 0.03264 |
| A1ZM043652_S_AT | NC0173626 | 8 | 96.8 | NC0199770 | 8 | 96.2 | trans | 0.02609 |
| A1ZM043652_S_AT | NC0173626 | 8 | 96.8 | NC0199803 | 3 | 85.6 | trans | 0.03177 |
| A1ZM043652_S_AT | NC0173626 | 8 | 96.8 | NC0199896 | 8 | 96.4 | trans | −0.03513 |
| A1ZM056158_AT | NC0173626 | 8 | 96.8 | NC0013275 | 2 | 49.7 | trans | 0.04091 |
| A1ZM056158_AT | NC0173626 | 8 | 96.8 | NC0199443 | 8 | 72.9 | trans | 0.02786 |
| A1ZM056158_AT | NC0173626 | 8 | 96.8 | NC0199461 | 1 | 176.7 | trans | 0.02943 |
| A1ZM056158_AT | NC0173626 | 8 | 96.8 | NC0199730 | 8 | 79.6 | trans | 0.03418 |
| A1ZM056158_AT | NC0173626 | 8 | 96.8 | NC0199735 | 8 | 84.4 | trans | 0.03264 |
| A1ZM056158_AT | NC0173626 | 8 | 96.8 | NC0199770 | 8 | 96.2 | trans | 0.02609 |
| A1ZM056158_AT | NC0173626 | 8 | 96.8 | NC0199896 | 8 | 96.4 | trans | −0.03513 |
| A1ZM001187_A_AT | NC0111472 | 8 | 103.3 | NC0000399 | 3 | 121.1 | trans | 0.03644 |
| A1ZM001187_A_AT | NC0111472 | 8 | 103.3 | NC0013275 | 2 | 49.7 | trans | 0.04091 |
| A1ZM001187_A_AT | NC0111472 | 8 | 103.3 | NC0015965 | 3 | 140.8 | trans | 0.03574 |
| A1ZM001187_A_AT | NC0111472 | 8 | 103.3 | NC0033526 | 8 | 153.1 | trans | 0.03979 |
| A1ZM001187_A_AT | NC0111472 | 8 | 103.3 | NC0112924 | 3 | 145.3 | trans | 0.02881 |
| A1ZM001187_A_AT | NC0111472 | 8 | 103.3 | NC0173436 | 3 | 86 | trans | 0.04209 |
| A1ZM001187_A_AT | NC0111472 | 8 | 103.3 | NC0173900 | 6 | 38.4 | trans | 0.03178 |
| A1ZM001187_A_AT | NC0111472 | 8 | 103.3 | NC0196949 | 2 | 43.4 | trans | 0.04475 |
| A1ZM001187_A_AT | NC0111472 | 8 | 103.3 | NC0199339 | 7 | 150.8 | trans | −0.02795 |
| A1ZM001187_A_AT | NC0111472 | 8 | 103.3 | NC0199461 | 1 | 176.7 | trans | 0.02943 |
| A1ZM001187_A_AT | NC0111472 | 8 | 103.3 | NC0199730 | 8 | 79.6 | trans | 0.03418 |
| A1ZM001187_A_AT | NC0111472 | 8 | 103.3 | NC0199735 | 8 | 84.4 | trans | 0.03264 |
| A1ZM001187_A_AT | NC0111472 | 8 | 103.3 | NC0199770 | 8 | 96.2 | trans | 0.02609 |
| A1ZM001187_A_AT | NC0111472 | 8 | 103.3 | NC0199803 | 3 | 85.6 | trans | 0.03177 |
| A1ZM001187_A_AT | NC0111472 | 8 | 103.3 | NC0199896 | 8 | 96.4 | trans | −0.03513 |
| A1ZM072906_AT | NC0199563 | 8 | 115.6 | NC0013275 | 2 | 49.7 | trans | 0.04091 |
| A1ZM004376_S_AT | NC0035106 | 8 | 151.4 | NC0000399 | 3 | 121.1 | trans | 0.03644 |
| A1ZM004376_S_AT | NC0035106 | 8 | 151.4 | NC0014545 | 8 | 155.1 | trans | 0.03338 |
| A1ZM004376_S_AT | NC0035106 | 8 | 151.4 | NC0033526 | 8 | 153.1 | trans | 0.03979 |
| A1ZM004376_S_AT | NC0035106 | 8 | 151.4 | NC0190032 | 3 | 147.8 | trans | 0.03234 |
| A1ZM017348_S_AT | NC0033526 | 8 | 153.1 | NC0000399 | 3 | 121.1 | trans | 0.03644 |
| A1ZM017348_S_AT | NC0033526 | 8 | 153.1 | NC0013275 | 2 | 49.7 | trans | 0.04091 |
| A1ZM017348_S_AT | NC0033526 | 8 | 153.1 | NC0014545 | 8 | 155.1 | trans | 0.03338 |
| A1ZM017348_S_AT | NC0033526 | 8 | 153.1 | NC0015965 | 3 | 140.8 | trans | 0.03574 |
| A1ZM017348_S_AT | NC0033526 | 8 | 153.1 | NC0020696 | 6 | 38.5 | trans | −0.03098 |
| A1ZM017348_S_AT | NC0033526 | 8 | 153.1 | NC0033526 | 8 | 153.1 | cis | 0.03979 |
| A1ZM017348_S_AT | NC0033526 | 8 | 153.1 | NC0112924 | 3 | 145.3 | trans | 0.02881 |
| A1ZM017348_S_AT | NC0033526 | 8 | 153.1 | NC0173900 | 6 | 38.4 | trans | 0.03178 |
| A1ZM017348_S_AT | NC0033526 | 8 | 153.1 | NC0190032 | 3 | 147.8 | trans | 0.03234 |
| A1ZM017348_S_AT | NC0033526 | 8 | 153.1 | NC0196949 | 2 | 43.4 | trans | 0.04475 |
| A1ZM017348_S_AT | NC0033526 | 8 | 153.1 | NC0199331 | 7 | 173.1 | trans | −0.02967 |
| A1ZM017348_S_AT | NC0033526 | 8 | 153.1 | NC0199443 | 8 | 72.9 | trans | 0.02786 |
| A1ZM017348_S_AT | NC0033526 | 8 | 153.1 | NC0199447 | 5 | 80.6 | trans | 0.03542 |
| A1ZM017348_S_AT | NC0033526 | 8 | 153.1 | NC0199461 | 1 | 176.7 | trans | 0.02943 |
| A1ZM017348_S_AT | NC0033526 | 8 | 153.1 | NC0199675 | 5 | 45.9 | trans | 0.02662 |
| A1ZM017348_S_AT | NC0033526 | 8 | 153.1 | NC0199730 | 8 | 79.6 | trans | 0.03418 |
| A1ZM017348_S_AT | NC0033526 | 8 | 153.1 | NC0199741 | 3 | 125.7 | trans | 0.02570 |
| A1ZM017348_S_AT | NC0033526 | 8 | 153.1 | NC0199760 | 1 | 179.1 | trans | 0.02836 |
| A1ZM017348_S_AT | NC0033526 | 8 | 153.1 | NC0199794 | 3 | 146 | trans | 0.04156 |
| A1ZM017348_S_AT | NC0033526 | 8 | 153.1 | NC0199896 | 8 | 96.4 | trans | −0.03513 |
| A1ZM017348_S_AT | NC0033526 | 8 | 153.1 | NC0199956 | 8 | 169.9 | trans | −0.03169 |
| A1ZM049860_AT | NC0033526 | 8 | 153.1 | NC0000399 | 3 | 121.1 | trans | 0.03644 |
| A1ZM049860_AT | NC0033526 | 8 | 153.1 | NC0014545 | 8 | 155.1 | trans | 0.03338 |
| A1ZM049860_AT | NC0033526 | 8 | 153.1 | NC0033526 | 8 | 153.1 | cis | 0.03979 |
| A1ZM049860_AT | NC0033526 | 8 | 153.1 | NC0036726 | 1 | 35.2 | trans | 0.02779 |
| A1ZM049860_AT | NC0033526 | 8 | 153.1 | NC0112924 | 3 | 145.3 | trans | 0.02881 |
| A1ZM049860_AT | NC0033526 | 8 | 153.1 | NC0196949 | 2 | 43.4 | trans | 0.04475 |
| A1ZM049860_AT | NC0033526 | 8 | 153.1 | NC0199447 | 5 | 80.6 | trans | 0.03542 |
| A1ZM049860_AT | NC0033526 | 8 | 153.1 | NC0199461 | 1 | 176.7 | trans | 0.02943 |
| A1ZM049860_AT | NC0033526 | 8 | 153.1 | NC0199555 | 1 | 34.4 | trans | 0.03511 |
| A1ZM049860_AT | NC0033526 | 8 | 153.1 | NC0199675 | 5 | 45.9 | trans | 0.02662 |
| A1ZM049860_AT | NC0033526 | 8 | 153.1 | NC0199730 | 8 | 79.6 | trans | 0.03418 |
| A1ZM032249_AT | NC0173530 | 9 | 78.1 | NC0015965 | 3 | 140.8 | trans | 0.03574 |
| A1ZM032249_AT | NC0173530 | 9 | 78.1 | NC0173530 | 9 | 78.1 | cis | −0.02725 |
| A1ZM032249_AT | NC0173530 | 9 | 78.1 | NC0190032 | 3 | 147.8 | trans | 0.03234 |
| A1ZM032249_AT | NC0173530 | 9 | 78.1 | NC0199346 | 2 | 50.8 | trans | 0.03424 |
| A1ZM032249_AT | NC0173530 | 9 | 78.1 | NC0199722 | 9 | 79.6 | trans | −0.03108 |
| A1ZM032249_AT | NC0173530 | 9 | 78.1 | NC0199794 | 3 | 146 | trans | 0.04156 |
| A1ZM084532_AT | NC0173530 | 9 | 78.1 | NC0015965 | 3 | 140.8 | trans | 0.03574 |
| A1ZM084532_AT | NC0173530 | 9 | 78.1 | NC0112924 | 3 | 145.3 | trans | 0.02881 |
| A1ZM084532_AT | NC0173530 | 9 | 78.1 | NC0173436 | 3 | 86 | trans | 0.04209 |
| A1ZM084532_AT | NC0173530 | 9 | 78.1 | NC0173530 | 9 | 78.1 | cis | −0.02725 |
| A1ZM084532_AT | NC0173530 | 9 | 78.1 | NC0196949 | 2 | 43.4 | trans | 0.04475 |
| A1ZM084532_AT | NC0173530 | 9 | 78.1 | NC0199722 | 9 | 79.6 | trans | −0.03108 |
| A1ZM084532_AT | NC0173530 | 9 | 78.1 | NC0199794 | 3 | 146 | trans | 0.04156 |
| A1ZM084532_AT | NC0173530 | 9 | 78.1 | NC0199896 | 8 | 96.4 | trans | −0.03513 |
| A1ZM026053_AT | NC0199722 | 9 | 79.6 | NC0013275 | 2 | 49.7 | trans | 0.04091 |
| A1ZM026053_AT | NC0199722 | 9 | 79.6 | NC0015965 | 3 | 140.8 | trans | 0.03574 |
| A1ZM026053_AT | NC0199722 | 9 | 79.6 | NC0036726 | 1 | 35.2 | trans | 0.02779 |
| A1ZM026053_AT | NC0199722 | 9 | 79.6 | NC0173530 | 9 | 78.1 | trans | −0.02725 |
| A1ZM026053_AT | NC0199722 | 9 | 79.6 | NC0190032 | 3 | 147.8 | trans | 0.03234 |
| A1ZM026053_AT | NC0199722 | 9 | 79.6 | NC0196949 | 2 | 43.4 | trans | 0.04475 |
| A1ZM026053_AT | NC0199722 | 9 | 79.6 | NC0199722 | 9 | 79.6 | cis | −0.03108 |
| A1ZM026053_AT | NC0199722 | 9 | 79.6 | NC0199741 | 3 | 125.7 | trans | 0.02570 |
| A1ZM026053_AT | NC0199722 | 9 | 79.6 | NC0199794 | 3 | 146 | trans | 0.04156 |
| A1ZM031333_AT | NC0199722 | 9 | 79.6 | NC0013275 | 2 | 49.7 | trans | 0.04091 |
| A1ZM031333_AT | NC0199722 | 9 | 79.6 | NC0015965 | 3 | 140.8 | trans | 0.03574 |
| A1ZM031333_AT | NC0199722 | 9 | 79.6 | NC0173530 | 9 | 78.1 | trans | −0.02725 |
| A1ZM031333_AT | NC0199722 | 9 | 79.6 | NC0199346 | 2 | 50.8 | trans | 0.03424 |
| A1ZM031333_AT | NC0199722 | 9 | 79.6 | NC0199722 | 9 | 79.6 | cis | −0.03108 |
| A1ZM031333_AT | NC0199722 | 9 | 79.6 | NC0199794 | 3 | 146 | trans | 0.04156 |
| A1ZM031333_AT | NC0199722 | 9 | 79.6 | NC0200185 | 7 | 147.7 | trans | 0.02563 |
| A1ZM033611_AT | NC0014240 | 9 | 103.5 | NC0002630 | 2 | 54.1 | trans | −0.02730 |
| A1ZM033611_AT | NC0014240 | 9 | 103.5 | NC0112924 | 3 | 145.3 | trans | 0.02881 |
| A1ZM033611_AT | NC0014240 | 9 | 103.5 | NC0173530 | 9 | 78.1 | trans | −0.02725 |
| A1ZM033611_AT | NC0014240 | 9 | 103.5 | NC0199346 | 2 | 50.8 | trans | 0.03424 |
| A1ZM033611_AT | NC0014240 | 9 | 103.5 | NC0199555 | 1 | 34.4 | trans | 0.03511 |
| A1ZM033611_AT | NC0014240 | 9 | 103.5 | NC0199722 | 9 | 79.6 | trans | −0.03108 |
| A1ZM067740_X_AT | NC0014240 | 9 | 103.5 | NC0173530 | 9 | 78.1 | trans | −0.02725 |
| A1ZM067740_X_AT | NC0014240 | 9 | 103.5 | NC0199346 | 2 | 50.8 | trans | 0.03424 |
| A1ZM067740_X_AT | NC0014240 | 9 | 103.5 | NC0199722 | 9 | 79.6 | trans | −0.03108 |
| A1ZM043521_AT | NC0199635 | 10 | 106.4 | NC0000129 | 8 | 16.5 | trans | 0.02884 |
| A1ZM043521_AT | NC0199635 | 10 | 106.4 | NC0000399 | 3 | 121.1 | trans | 0.03644 |
| A1ZM043521_AT | NC0199635 | 10 | 106.4 | NC0015965 | 3 | 140.8 | trans | 0.03574 |
| A1ZM043521_AT | NC0199635 | 10 | 106.4 | NC0112924 | 3 | 145.3 | trans | 0.02881 |
| A1ZM043521_AT | NC0199635 | 10 | 106.4 | NC0173530 | 9 | 78.1 | trans | −0.02725 |
| A1ZM043521_AT | NC0199635 | 10 | 106.4 | NC0173859 | 7 | 96.8 | trans | 0.02582 |
| A1ZM043521_AT | NC0199635 | 10 | 106.4 | NC0199722 | 9 | 79.6 | trans | −0.03108 |
| A1ZM043521_AT | NC0199635 | 10 | 106.4 | NC0199741 | 3 | 125.7 | trans | 0.02570 |
| A1ZM043521_AT | NC0199635 | 10 | 106.4 | NC0200044 | 1 | 47.5 | trans | −0.03558 |
| A1ZM043521_AT | NC0199635 | 10 | 106.4 | NC0200185 | 7 | 147.7 | trans | 0.02563 |

Table 9 provides a summary of the significant markers, including the corresponding SEQ ID No.

TABLE 9
Summary of SNP markers associated with yield eQTL in cis, trans, or
cis and trans.

| SEQ | | | | |
|---|---|---|---|---|
| ID | | | | PROBESET |
| NO | MARKER | CHROMOSOME | POSITION | ORIENTATION |
| 1 | NC0019524 | 1 | 5.8 | trans |
| 2 | NC0039304 | 1 | 19.7 | trans |
| 3 | NC0070876 | 1 | 19.7 | cis |
| 4 | NC0173555 | 1 | 22.9 | trans |
| 5 | NC0199703 | 1 | 26.9 | trans |
| 6 | NC0173400 | 1 | 32.2 | trans + cis |
| 7 | NC0173332 | 1 | 33.3 | trans |
| 8 | NC0199555 | 1 | 34.4 | trans + cis |
| 9 | NC0035426 | 1 | 34.5 | trans |
| 10 | NC0036726 | 1 | 35.2 | trans |
| 11 | NC0144205 | 1 | 38.5 | trans |
| 12 | NC0175336 | 1 | 43.5 | trans |
| 13 | NC0173858 | 1 | 43.8 | trans |
| 14 | NC0200188 | 1 | 44.1 | trans |
| 15 | NC0200044 | 1 | 47.5 | trans |
| 16 | NC0143873 | 1 | 52.6 | trans |
| 17 | NC0199417 | 1 | 54.3 | trans |
| 18 | NC0010830 | 1 | 65.8 | trans |
| 19 | NC0199971 | 1 | 66.1 | trans |
| 20 | NC0158866 | 1 | 66.4 | trans |
| 21 | NC0200213 | 1 | 67.4 | trans + cis |
| 22 | NC0199911 | 1 | 70.2 | trans |
| 23 | NC0043266 | 1 | 71.3 | trans |
| 24 | NC0199318 | 1 | 85.9 | trans |
| 25 | NC0157255 | 1 | 87.7 | trans |
| 26 | NC0157397 | 1 | 87.7 | trans |
| 27 | NC0029053 | 1 | 88.1 | trans |
| 28 | NC0171154 | 1 | 88.1 | trans |
| 29 | NC0032745 | 1 | 94.5 | trans |
| 30 | NC0173334 | 1 | 102.8 | trans |
| 31 | NC0173484 | 1 | 103.2 | trans + cis |
| 32 | NC0199391 | 1 | 107.5 | trans |
| 33 | NC0188455 | 1 | 113.3 | trans |
| 34 | NC0199986 | 1 | 113.3 | trans |
| 35 | NC0199454 | 1 | 136.7 | trans |
| 36 | NC0170811 | 1 | 153.9 | trans |
| 37 | NC0173449 | 1 | 154.4 | trans |
| 38 | NC0173453 | 1 | 172.2 | trans |
| 39 | NC0199461 | 1 | 176.7 | trans + cis |
| 40 | NC0199760 | 1 | 179.1 | trans |
| 41 | NC0200153 | 1 | 182.8 | trans |
| 42 | NC0199491 | 1 | 183.9 | trans |
| 43 | NC0200135 | 1 | 188.2 | trans |
| 44 | NC0015937 | 1 | 197.7 | trans |
| 45 | NC0173395 | 1 | 200.1 | trans |
| 46 | NC0196534 | 1 | 201.9 | trans |
| 47 | NC0199661 | 1 | 202.9 | trans |
| 48 | NC0200158 | 1 | 205.1 | trans |
| 49 | NC0188030 | 1 | 207.9 | trans |
| 50 | NC0199556 | 1 | 208 | trans |
| 51 | NC0009148 | 1 | 213 | trans |
| 52 | NC0173402 | 1 | 217 | trans |
| 53 | NC0173951 | 1 | 229.4 | trans + cis |
| 54 | NC0200048 | 1 | 230.2 | trans |
| 55 | NC0200138 | 1 | 238.1 | trans |
| 56 | NC0112831 | 2 | 9 | trans |
| 57 | NC0199738 | 2 | 28.8 | trans |
| 58 | NC0173409 | 2 | 32.7 | trans |
| 59 | NC0080029 | 2 | 33.1 | trans |
| 60 | NC0196898 | 2 | 33.5 | trans |
| 61 | NC0196949 | 2 | 43.4 | trans |
| 62 | NC0013275 | 2 | 49.7 | trans + cis |
| 63 | NC0199346 | 2 | 50.8 | trans + cis |
| 64 | NC0002630 | 2 | 54.1 | trans |
| 65 | NC0199954 | 2 | 54.1 | trans |
| 66 | NC0027262 | 2 | 57.3 | trans |
| 67 | NC0199350 | 2 | 67.8 | trans |
| 68 | NC0003725 | 2 | 68.4 | trans |
| 69 | NC0196659 | 2 | 70.4 | trans |
| 70 | NC0199310 | 2 | 75.7 | trans |
| 71 | NC0199415 | 2 | 84.7 | trans |
| 72 | NC0170324 | 2 | 94 | trans |
| 73 | NC0172302 | 2 | 94 | trans |
| 74 | NC0174027 | 2 | 99.2 | trans |
| 75 | NC0200140 | 2 | 99.3 | trans |
| 76 | NC0199297 | 2 | 100.2 | trans |
| 77 | NC0199568 | 2 | 114.7 | trans |
| 78 | NC0035094 | 2 | 116.9 | trans |
| 79 | NC0174061 | 2 | 118.1 | trans |
| 80 | NC0200164 | 2 | 120.4 | trans |
| 81 | NC0199511 | 2 | 121.3 | trans |
| 82 | NC0038699 | 2 | 121.8 | trans |
| 83 | NC0174062 | 2 | 129.5 | trans + cis |
| 84 | NC0004901 | 2 | 131.6 | trans |
| 85 | NC0188475 | 2 | 131.6 | trans |
| 86 | NC0193437 | 2 | 131.6 | trans |
| 87 | NC0199317 | 2 | 138.2 | trans |
| 88 | NC0173731 | 2 | 138.4 | trans |
| 89 | NC0199713 | 2 | 139.5 | trans |
| 90 | NC0199524 | 2 | 141.5 | trans |
| 91 | NC0199851 | 2 | 165.2 | trans |
| 92 | NC0033320 | 2 | 173.6 | trans + cis |
| 93 | NC0110714 | 2 | 190.1 | trans |
| 94 | NC0199645 | 3 | 12.1 | trans |
| 95 | NC0003536 | 3 | 30.9 | trans + cis |
| 96 | NC0051616 | 3 | 40.4 | trans |
| 97 | NC0195038 | 3 | 74.2 | trans |
| 98 | NC0109315 | 3 | 77.4 | trans + cis |
| 99 | NC0173641 | 3 | 80.9 | trans |
| 100 | NC0199465 | 3 | 81.6 | trans |
| 101 | NC0199853 | 3 | 81.6 | trans |
| 102 | NC0200126 | 3 | 81.8 | trans |
| 103 | NC0173790 | 3 | 83.2 | trans |
| 104 | NC0199803 | 3 | 85.6 | trans |
| 105 | NC0173436 | 3 | 86 | trans + cis |
| 106 | NC0199763 | 3 | 86 | trans |
| 107 | NC0199947 | 3 | 86.4 | trans |
| 108 | NC0030963 | 3 | 87 | trans |
| 109 | NC0173845 | 3 | 87 | trans |
| 110 | NC0199372 | 3 | 87.1 | trans |
| 111 | NC0009470 | 3 | 88 | trans |
| 112 | NC0199522 | 3 | 90.3 | trans |
| 113 | NC0199589 | 3 | 91.5 | trans + cis |
| 114 | NC0199676 | 3 | 91.5 | trans |
| 115 | NC0200156 | 3 | 96.1 | trans + cis |
| 116 | NC0198241 | 3 | 102 | trans |
| 117 | NC0200081 | 3 | 115.9 | trans |
| 118 | NC0173739 | 3 | 116.9 | trans |
| 119 | NC0000399 | 3 | 121.1 | trans |
| 120 | NC0199669 | 3 | 122.8 | trans |
| 121 | NC0199741 | 3 | 125.7 | trans + cis |
| 122 | NC0015965 | 3 | 140.8 | trans |
| 123 | NC0199921 | 3 | 141.1 | trans |
| 124 | NC0112924 | 3 | 145.3 | trans + cis |
| 125 | NC0199794 | 3 | 146 | trans |
| 126 | NC0190032 | 3 | 147.8 | trans |
| 127 | NC0199913 | 3 | 152.8 | trans |
| 128 | NC0035568 | 3 | 166.8 | trans |
| 129 | NC0174066 | 3 | 168.4 | trans |
| 130 | NC0190159 | 3 | 168.4 | trans |
| 131 | NC0199485 | 3 | 175.1 | trans |
| 132 | NC0112481 | 3 | 183.6 | trans |
| 133 | NC0199736 | 3 | 187.4 | trans |
| 134 | NC0188678 | 3 | 201.5 | trans |
| 135 | NC0018073 | 3 | 201.6 | trans |
| 136 | NC0190645 | 3 | 202.4 | trans |
| 137 | NC0199814 | 3 | 208.5 | trans |
| 138 | NC0000189 | 3 | 215.3 | trans |
| 139 | NC0193457 | 3 | 216.6 | trans + cis |
| 140 | NC0170468 | 3 | 216.7 | trans |
| 141 | NC0195163 | 3 | 216.7 | trans |
| 142 | NC0199657 | 4 | 0.5 | trans |
| 143 | NC0199824 | 4 | 13.6 | trans |
| 144 | NC0175374 | 4 | 21.6 | trans |
| 145 | NC0199837 | 4 | 46.8 | trans |
| 146 | NC0077263 | 4 | 50.6 | trans |
| 147 | NC0067577 | 4 | 59.3 | trans |
| 148 | NC0010671 | 4 | 63.7 | trans |
| 149 | NC0199978 | 4 | 70.6 | trans |
| 150 | NC0199812 | 4 | 77.3 | trans |
| 151 | NC0171755 | 4 | 77.8 | trans |
| 152 | NC0173414 | 4 | 78.9 | trans |
| 153 | NC0199603 | 4 | 79.8 | trans |
| 154 | NC0028257 | 4 | 81.8 | trans |
| 155 | NC0199680 | 4 | 81.9 | trans |
| 156 | NC0199410 | 4 | 82.5 | trans |
| 157 | NC0199781 | 4 | 82.7 | trans |
| 158 | NC0200151 | 4 | 84.4 | trans |
| 159 | NC0199832 | 4 | 84.7 | trans |
| 160 | NC0200083 | 4 | 84.9 | trans |
| 161 | NC0187941 | 4 | 121 | trans |
| 162 | NC0199877 | 4 | 127.9 | trans |
| 163 | NC0200132 | 4 | 128.7 | trans |
| 164 | NC0038211 | 4 | 141.1 | trans |
| 165 | NC0173919 | 4 | 142.6 | trans |
| 166 | NC0199575 | 4 | 142.6 | trans |
| 167 | NC0199694 | 4 | 142.6 | trans |
| 168 | NC0173628 | 4 | 144 | trans |
| 169 | NC0199707 | 4 | 144 | trans |
| 170 | NC0199373 | 4 | 146.5 | trans |
| 171 | NC0199771 | 4 | 147.3 | trans |
| 172 | NC0173568 | 4 | 150.5 | trans |
| 173 | NC0173518 | 4 | 152.5 | trans |
| 174 | NC0199421 | 4 | 156.3 | trans |
| 175 | NC0024368 | 4 | 162.6 | trans |
| 176 | NC0029487 | 4 | 171.1 | trans |
| 177 | NC0199468 | 4 | 173.5 | trans |
| 178 | NC0189286 | 4 | 177 | trans |
| 179 | NC0147215 | 4 | 179.6 | trans |
| 180 | NC0199826 | 4 | 190.8 | trans |
| 181 | NC0174148 | 5 | 1.8 | trans |
| 182 | NC0199470 | 5 | 2 | trans |
| 183 | NC0199442 | 5 | 22.4 | trans |
| 184 | NC0011194 | 5 | 29.3 | trans |
| 185 | NC0199675 | 5 | 45.9 | trans |
| 186 | NC0199376 | 5 | 54.4 | trans |
| 187 | NC0199293 | 5 | 62.9 | trans |
| 188 | NC0199739 | 5 | 62.9 | trans |
| 189 | NC0079519 | 5 | 63.1 | trans |
| 190 | NC0189552 | 5 | 63.8 | trans |
| 191 | NC0195197 | 5 | 66.6 | trans |
| 192 | NC0200171 | 5 | 73.2 | trans |
| 193 | NC0056874 | 5 | 73.5 | trans |
| 194 | NC0200120 | 5 | 75.4 | trans |
| 195 | NC0199447 | 5 | 80.6 | trans |
| 196 | NC0173716 | 5 | 81.5 | trans |
| 197 | NC0188018 | 5 | 85.4 | trans |
| 198 | NC0192676 | 5 | 88.2 | trans |
| 199 | NC0194693 | 5 | 89.9 | trans |
| 200 | NC0036637 | 5 | 100 | trans |
| 201 | NC0012281 | 5 | 106.5 | trans |
| 202 | NC0200106 | 5 | 124.4 | trans |
| 203 | NC0079526 | 5 | 125 | trans |
| 204 | NC0200093 | 5 | 131.8 | trans + cis |
| 205 | NC0107877 | 5 | 132.7 | trans |
| 206 | NC0200147 | 5 | 139.5 | trans |
| 207 | NC0078090 | 5 | 141.8 | trans |
| 208 | NC0173396 | 5 | 143.1 | trans |
| 209 | NC0042059 | 5 | 143.2 | trans |
| 210 | NC0173843 | 5 | 143.2 | trans |
| 211 | NC0200099 | 5 | 171.1 | trans |
| 212 | NC0200212 | 5 | 176.4 | trans |
| 213 | NC0173452 | 5 | 177.7 | trans |
| 214 | NC0033924 | 6 | 36.2 | trans |
| 215 | NC0042917 | 6 | 36.4 | trans |
| 216 | NC0173900 | 6 | 38.4 | trans |
| 217 | NC0194397 | 6 | 38.4 | trans |
| 218 | NC0020696 | 6 | 38.5 | trans |
| 219 | NC0199945 | 6 | 39.4 | trans |
| 220 | NC0199422 | 6 | 41.1 | trans |
| 221 | NC0036529 | 6 | 43.2 | trans + cis |
| 222 | NC0028658 | 6 | 52.7 | cis |
| 223 | NC0029612 | 6 | 57.8 | trans |
| 224 | NC0143950 | 6 | 66 | trans |
| 225 | NC0158130 | 6 | 66.3 | trans |
| 226 | NC0196774 | 6 | 69.4 | trans |
| 227 | NC0173525 | 6 | 70.2 | trans |
| 228 | NC0199455 | 6 | 73.3 | trans |
| 229 | NC0199535 | 6 | 75.4 | trans |
| 230 | NC0199750 | 6 | 81.6 | trans |
| 231 | NC0199915 | 6 | 84.9 | trans |
| 232 | NC0173962 | 6 | 96.7 | trans |
| 233 | NC0199480 | 6 | 99.9 | trans |
| 234 | NC0200165 | 6 | 99.9 | trans |
| 235 | NC0173876 | 6 | 104.2 | trans |
| 236 | NC0200112 | 6 | 105.6 | trans |
| 237 | NC0195223 | 6 | 118.6 | trans + cis |
| 238 | NC0199409 | 6 | 118.6 | trans |
| 239 | NC0200186 | 6 | 131 | trans |
| 240 | NC0173794 | 6 | 131.1 | trans |
| 241 | NC0013445 | 6 | 131.2 | trans |
| 242 | NC0173500 | 6 | 136 | trans |
| 243 | NC0199958 | 6 | 142 | trans + cis |
| 244 | NC0194601 | 6 | 143.7 | trans |
| 245 | NC0194906 | 6 | 144.8 | trans |
| 246 | NC0173584 | 7 | 8.5 | trans |
| 247 | NC0020611 | 7 | 10 | trans |
| 248 | NC0174551 | 7 | 11.2 | trans |
| 249 | NC0200199 | 7 | 12.4 | trans |
| 250 | NC0158250 | 7 | 24.3 | trans |
| 251 | NC0199685 | 7 | 28.6 | trans |
| 252 | NC0199937 | 7 | 31.2 | trans |
| 253 | NC0199723 | 7 | 40.4 | trans |
| 254 | NC0173476 | 7 | 44.2 | trans |
| 255 | NC0174167 | 7 | 58 | trans |
| 256 | NC0199984 | 7 | 60.4 | trans |
| 257 | NC0199648 | 7 | 62.5 | trans |
| 258 | NC0199388 | 7 | 62.8 | trans |
| 259 | NC0173493 | 7 | 62.9 | trans |
| 260 | NC0040322 | 7 | 64.5 | trans |
| 261 | NC0042930 | 7 | 64.5 | trans |
| 262 | NC0200064 | 7 | 65.3 | trans |
| 263 | NC0199312 | 7 | 65.9 | trans |
| 264 | NC0173435 | 7 | 71.6 | trans |
| 265 | NC0199754 | 7 | 77.6 | trans |
| 266 | NC0200207 | 7 | 84.1 | trans |
| 267 | NC0008799 | 7 | 87 | trans |
| 268 | NC0199354 | 7 | 87.4 | trans |
| 269 | NC0173859 | 7 | 96.8 | trans + cis |
| 270 | NC0015314 | 7 | 99 | trans |
| 271 | NC0071001 | 7 | 99.4 | trans |
| 272 | NC0199889 | 7 | 99.4 | trans |
| 273 | NC0038733 | 7 | 106.5 | trans + cis |
| 274 | NC0200123 | 7 | 107.2 | trans |
| 275 | NC0199614 | 7 | 108.4 | trans |
| 276 | NC0079306 | 7 | 111.7 | trans |
| 277 | NC0199879 | 7 | 112.1 | trans |
| 278 | NC0199800 | 7 | 114.5 | trans |
| 279 | NC0173358 | 7 | 131 | trans |
| 280 | NC0199355 | 7 | 138.4 | trans |
| 281 | NC0200185 | 7 | 147.7 | trans + cis |
| 282 | NC0199339 | 7 | 150.8 | trans |
| 283 | NC0173751 | 7 | 152.4 | trans |
| 284 | NC0187978 | 7 | 165.7 | trans |
| 285 | NC0199331 | 7 | 173.1 | trans |
| 286 | NC0000129 | 8 | 16.5 | trans |
| 287 | NC0049890 | 8 | 41.2 | trans |
| 288 | NC0199805 | 8 | 51.7 | trans |
| 289 | NC0200039 | 8 | 55.3 | trans |
| 290 | NC0173490 | 8 | 59 | trans |
| 291 | NC0030011 | 8 | 63.1 | trans |
| 292 | NC0079084 | 8 | 65.2 | trans |
| 293 | NC0200128 | 8 | 65.7 | trans |
| 294 | NC0171070 | 8 | 70 | trans |
| 295 | NC0173531 | 8 | 70 | trans |
| 296 | NC0199443 | 8 | 72.9 | trans + cis |
| 297 | NC0113218 | 8 | 75.2 | trans |
| 298 | NC0194645 | 8 | 76.5 | trans |
| 299 | NC0199730 | 8 | 79.6 | trans + cis |
| 300 | NC0199834 | 8 | 84 | trans |
| 301 | NC0199735 | 8 | 84.4 | trans + cis |
| 302 | NC0199440 | 8 | 87.9 | trans |
| 303 | NC0199313 | 8 | 89.3 | trans |
| 304 | NC0145457 | 8 | 91.1 | trans |
| 305 | NC0174094 | 8 | 91.1 | trans |
| 306 | NC0200184 | 8 | 91.1 | trans |
| 307 | NC0199581 | 8 | 91.6 | trans |
| 308 | NC0199860 | 8 | 93.9 | trans |
| 309 | NC0151542 | 8 | 96.2 | trans |
| 310 | NC0199770 | 8 | 96.2 | trans |
| 311 | NC0199896 | 8 | 96.4 | trans + cis |
| 312 | NC0173626 | 8 | 96.8 | trans + cis |
| 313 | NC0111472 | 8 | 103.3 | trans + cis |
| 314 | NC0173350 | 8 | 104 | trans |
| 315 | NC0199563 | 8 | 115.6 | trans + cis |
| 316 | NC0158373 | 8 | 148.9 | trans |
| 317 | NC0035106 | 8 | 151.4 | trans + cis |
| 318 | NC0033526 | 8 | 153.1 | trans + cis |
| 319 | NC0014545 | 8 | 155.1 | trans |
| 320 | NC0199880 | 8 | 161.8 | trans |
| 321 | NC0199956 | 8 | 169.9 | trans |
| 322 | NC0010740 | 9 | 0 | trans |
| 323 | NC0056940 | 9 | 0.4 | trans |
| 324 | NC0199387 | 9 | 33 | trans |
| 325 | NC0173574 | 9 | 36.6 | trans |
| 326 | NC0157736 | 9 | 56.6 | trans |
| 327 | NC0200122 | 9 | 56.6 | trans |
| 328 | NC0174063 | 9 | 60.8 | trans |
| 329 | NC0200107 | 9 | 65.9 | trans |
| 330 | NC0199464 | 9 | 66.5 | trans |
| 331 | NC0002876 | 9 | 66.6 | trans |
| 332 | NC0029744 | 9 | 67.4 | trans |
| 333 | NC0173953 | 9 | 67.4 | trans |
| 334 | NC0107256 | 9 | 68.5 | trans |
| 335 | NC0173799 | 9 | 68.5 | trans |
| 336 | NC0020872 | 9 | 76.3 | trans |
| 337 | NC0173530 | 9 | 78.1 | trans + cis |
| 338 | NC0199722 | 9 | 79.6 | trans + cis |
| 339 | NC0023268 | 9 | 84.5 | trans |
| 340 | NC0173571 | 9 | 90.4 | trans |
| 341 | NC0199525 | 9 | 90.9 | trans |
| 342 | NC0148189 | 9 | 91 | trans |
| 343 | NC0028613 | 9 | 94.6 | trans |
| 344 | NC0010789 | 9 | 96.9 | trans |
| 345 | NC0153579 | 9 | 97.3 | trans |
| 346 | NC0014240 | 9 | 103.5 | trans + cis |
| 347 | NC0030512 | 9 | 103.6 | trans |
| 348 | NC0200144 | 9 | 110.4 | trans |
| 349 | NC0051922 | 9 | 110.5 | trans |
| 350 | NC0029024 | 9 | 116.9 | trans |
| 351 | NC0199898 | 9 | 119.2 | trans |
| 352 | NC0173478 | 9 | 122.4 | trans |
| 353 | NC0174164 | 9 | 147.9 | trans |
| 354 | NC0173382 | 10 | 27.7 | trans |
| 355 | NC0158122 | 10 | 36.7 | trans |
| 356 | NC0199574 | 10 | 45.2 | trans |
| 357 | NC0157951 | 10 | 48.3 | trans |
| 358 | NC0199810 | 10 | 48.3 | trans |
| 359 | NC0173834 | 10 | 49.1 | trans |
| 360 | NC0199686 | 10 | 49.1 | trans |
| 361 | NC0199366 | 10 | 51.6 | trans |
| 362 | NC0053108 | 10 | 61.2 | trans |
| 363 | NC0173818 | 10 | 65.3 | trans |
| 364 | NC0011002 | 10 | 66.5 | trans |
| 365 | NC0197151 | 10 | 92.4 | trans |
| 366 | NC0199635 | 10 | 106.4 | trans + cis |

### EXAMPLE 2

### Association of Genetic Variation in Yield with Transcript Expression and Expression QTL Using Design II Crosses of Homozygous Inbred Lines of Maize

This example provides nucleic acid sequences associated with grain yield in maize. SEQ IDs are provided for the ORF corresponding to probesets associated with yield, wherein these nucleic acids are useful both as molecular markers for either SNP development or sequence-assisted breeding (U.S. Patent Application Ser. No. 60/942,707) and as nucleic acid expression constructs for use in transgenic breeding. Further, data are providing that support the value of these nucleic acid sequences for the prediction of yield, via correlation analyses for the probesets.

These yield-associated nucleic acids were identified from a transcript profiling experiment referred as Design II involving corn elite hybrid lines. The method uses inbreds which may or may not be related by descent and uniquely combine them in a Design II mating scheme to generate hybrids. The method further uses the hybrids derived by combining related inbreds to identify genetic gain for gene expression in relation to key economic traits like increased yield, nitrogen and stress tolerance.

The uniqueness of the design allows for the simultaneous evaluation of: general combining ability for gene expression in relation to key economic traits; specific combining ability for gene expression in relation to key economic traits; additivity in gene expression with respect to key economic traits; dominance in gene expression with respect to key economic traits; and heterosis and genetic gain for gene expression in relation to key economic traits. In addition, this design allows for identification of: key regulatory pathways affecting genes that impact key economic traits; mutations in genes leading to a change in gene expression in the process of breeding new inbreds which may contribute for a greater performance with respect to key economic traits; and genes which have a significantly different gene expression in the newer and more elite inbred lines (offspring) compared to parents based on differential gene expression in hybrids of which these parents are a part.

In Design II experiment, 30 hybrids from six female and five male inbred lines were grown in in three locations (JA, JE, and CA). Both leaf and kernel tissues were harvested at R2 and R4 stages for TxP experiment. In addition, kernel tissue at mature stage was also sampled at JA and JE locations for TxP. Hybrid yield data were collected on individual locations.

Correlation analysis was conducted between gene expression level and yield across the 30 hybrids by individual tissue and stage on each location. The genes showing the strongest correlations (both positive and negative) at all of the locations were selected. The cutoff for correlation coefficients was 0.5 or larger (−0.5 or lower) at each location. To this end, a total of 22 probesets were identified (5 positive, 16 negative). Very interestingly, 21 of the 22 strongest correlations are from the kernel rather than the leaf tissue.

These nucleic acid sequences are good candidates for use as markers in breeding as well as for testing in a corn transgenic pipeline. Biotechnological manipulation of these genes (Over expression, regulated expression or suppression or expression of protein variants) is expected to impart enhanced agronomic traits to plants e.g. increased yield, enhanced stress tolerance, increased nutrient utilization, increased seed oil etc.

TABLE 10
Summary of significant probesets and corresponding correlation to yield,
reported per location.

| | | | | | Location | | |
|---|---|---|---|---|---|---|---|
| | | SEQ | | | Correlation | | |
| TxP ID | Annotation | ID NO | Tissue | Stage | JA | JE | CA |
| A1ZM064837_at | OSJNBa0084A10.13 | 422 | KERNEL | R4 | −0.67 | −0.51 | −0.59 |
| | protein | | | | | | |
| A1ZM071589_at | Expressed | 423 | KERNEL | MAT | −0.51 | −0.61 | 0 |
| | protein | | | | | | |
| A1ZM017493_at | Hypothetical | 424 | KERNEL | R2 | −0.56 | −0.51 | −0.56 |
| | protein | | | | | | |
| | P0654A08.34 | | | | | | |
| A1ZM009253_at | Hypothetical | 425 | KERNEL | R2 | −0.54 | −0.53 | −0.55 |
| | protein | | | | | | |
| | OJ1254_E07.2-1 | | | | | | |
| A1ZM020055_at | Hypothetical | 426 | KERNEL | R2 | −0.52 | −0.54 | −0.58 |
| | protein | | | | | | |
| | OSJNBa0072I06.32 | | | | | | |
| A1ZM009058_at | Os05g0144000 | 427 | KERNEL | R4 | −0.56 | −0.51 | −0.5 |
| | protein | | | | | | |
| A1ZM000169_x_at | Endochitinase | 428 | KERNEL | MAT | −0.6 | −0.52 | 0 |
| | A precursor | | | | | | |
| A1ZM025213_at | Putative 60S | 429 | KERNEL | MAT | −0.54 | −0.55 | 0 |
| | ribosomal | | | | | | |
| | protein L38 | | | | | | |
| A1ZM033574_s_at | Os03g0825400 | 430 | KERNEL | MAT | −0.55 | −0.64 | 0 |
| | protein | | | | | | |
| A1ZM017424_at | Hypothetical | 431 | KERNEL | R4 | −0.7 | −0.51 | −0.6 |
| | protein | | | | | | |
| | OSJNBa0029C15.26 | | | | | | |
| A1ZM075788_at | Os03g0827700 | 432 | KERNEL | MAT | −0.51 | −0.58 | 0 |
| | protein | | | | | | |
| A1ZM027765_at | Cysteine-rich | 433 | KERNEL | MAT | −0.63 | −0.5 | 0 |
| | extensin-like | | | | | | |
| | protein-1 | | | | | | |
| A1ZM000477_a_at | Putative PHD- | 434 | KERNEL | MAT | 0.59 | 0.53 | 0 |
| | finger domain | | | | | | |
| | containing | | | | | | |
| | protein | | | | | | |
| A1ZM023275_at | Adenosine 5′- | 435 | LEAF | R4 | 0.57 | 0.5 | 0.66 |
| | phosphosulfate | | | | | | |
| | reductase 8 | | | | | | |
| A1ZM056190_at | B0806H07.1 | 436 | KERNEL | R2 | 0.56 | 0.51 | 0.54 |
| | protein | | | | | | |
| A1ZM005830_at | OSJNBa0091D06.3 | 437 | KERNEL | MAT | 0.54 | 0.53 | 0 |
| | protein | | | | | | |
| A1ZM011567_at | OSIGBa0092M08.10 | 438 | KERNEL | MAT | 0.57 | 0.57 | 0 |
| | protein | | | | | | |

### EXAMPLE 3

### Mining Rare Alleles

The use of expression profile data enables detection of rare alleles or haplotypes in the genome of a plant. This is particularly important for leveraging rare but important genomic regions in a breeding program, such as a disease resistance locus from exotic or unadapted germplasm, wherein rare alleles are defined as occurring in low frequency within the germplasm pool and potentially being previously undetected within the germplasm pool. The present example provides methods for rare allele detection, experimental design (i.e., selecting exotic germplasm, germplasm with known phenotype of interest, screening non-elite germplasm), and utility (i.e., introgression programs for beneficial rare variants for specific traits and/or to expand germplasm diversity in one or more specific germplasm pools such as per maturity zone).

A set of germplasm comprising at least 2 germplasm entries is provided. Non-limiting factors influencing inclusion in a sequencing project for at least one locus include germplasm origin or geography, at least one genotype of interest, at least one phenotype of interest, performance in hybrid crosses, performance of a transgene, and other observations of the germplasm or predictions relating the germplasm and its performance.

Using the methods and approaches presented herein, at least one base pair position is queried using isolated nucleic acids and expression profile technology for at least 2 germplasm entries. Using methods known in the art for expression profile statistical methods, bioinformatics, and in silico evaluation, differences and similarities are identified and linked to the source germplasm entry. Following identification of alleles of interest, selection decisions can be made.

In the case of rare allele mining, the rare allele may be associated with a known phenotype. In addition, the identification of the rare allele can provide the basis for additional phenotyping, association studies, and other assays to evaluate the effect of the rare allele on plant phenotype and breeding performance. Further, the nucleic acid sequence of the rare allele can be immediately leveraged for use as a marker via methods known in the art and described herein to detect this rare allele in additional germplasm entries, to be used as a basis for selection, and to facilitate introgression of the rare allele in germplasm entries lacking the rare allele. In other aspects, the rare allele is isolated and the isolated nucleic acid is transformed into a plant using methods known in the art in order to confer a preferred phenotype to the recipient plant. The recipient plant can subsequently be used as a donor for conversion programs to cross with elite germplasm for trait integration purposes.

The identification of rare alleles is useful for leveraging the full genetic potential of any germplasm pool, i.e., set of 2 or more germplasm entries. This is useful for determining breeding cross strategy, increasing the diversity between 2 or more germplasm pools, evaluating heterotic pools, and informing breeding decisions. High throughput sequencing both accelerates the identification of the alleles and allows simultaneous detection of rare alleles and identification of associated markers.

We claim:

1. A method of plant breeding comprising
a. providing at least two plants wherein at least one expression profile is assayed for at least one locus for at each plant;
b. determining an expression profile effect estimate for at least one phenotypic trait for at least two expression profiles for the at least one locus; and
c. making plant breeding decisions based on the determined expression profile effect estimate for the at least one phenotypic trait.

2. The method of claim 1, wherein the plant is selected from the group consisting of a forage crop, oilseed crop, grain crop, fruit crop, ornamental plants, vegetable crop, fiber crop, spice crop, nut crop, turf crop, sugar crop, beverage crop, tuber crop, root crop, and forest crop.

3. The method of claim 1, wherein the phenotypic trait is selected from the group consisting of herbicide tolerance, disease resistance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, sterility, other agronomic traits, traits for industrial uses, or traits for improved consumer appeal.

4. The method of claim 1, wherein the plant breeding decision comprises selecting among breeding populations based on the at least one numerical value.

5. The method of claim 1, wherein the plant breeding decision comprises selecting progeny in one or more breeding populations based on the at least one numerical value.

6. The method of claim 1, wherein the plant breeding decision comprises predicting progeny performance of parental lines and selecting parental lines based on the predicted progeny performance

7. The method of claim 1, wherein the plant breeding decision comprises advancing lines in germplasm improvement activities based on the at least one numerical value

8. The method of claim 7, wherein the germplasm improvement activities are selected are selected from the group consisting of line and variety development, hybrid development, transgenic event selection, making breeding crosses, testing and advancing a plant through self fertilization, purification of lines or sublines, using plant or parts thereof for transformation, using plants or parts thereof for candidates for expression constructs, and using plant or parts thereof for mutagenesis.

9. The method of claim 1, wherein the plant breeding decision comprises selecting for at least one phenotypic trait based on the at least one numerical value associated with another phenotypic trait.

10. The method of claim 1, wherein at least one expression profile is transcribed from a sequence selected from the group consisting of SEQ ID NO 1-438.

11. A method of plant breeding comprising
a. establishing a fingerprint map defining a plurality of loci within the genome of at least two plants;
b. determining a plurality of expression profile for the at least two plants;
c. associating a QTL allele of at least one locus with known map location with at least one phenotypic trait;
d. associating the QTL allele to at least one of the expression profiles to predict expression of the phenotypic trait; and
e. assaying at least one other plant for presence of the QTL allele using at least one marker.

12. The method of claim 11, wherein the marker is selected from the group consisting of a genetic marker, a haplotype, a nucleic acid sequence, a transcriptional profile, a metabolic profile, a nutrient composition profile, a protein expression profile, and a phenotypic character.

13. A method of marker assisted breeding comprising
a. providing a breeding population comprising at least two plants;
b. associating at least one phenotypic trait with at least one locus of the genome of the plants of the breeding population, wherein the locus is defined by at least one nucleic acid sequence; and
c. assaying for the presence of the at least one nucleic acid sequence of the at least one locus to predict expression of at least one phenotypic trait in a progeny plant of the breeding population.


SEQUENCE LISTING (438 sequences)

SEQ ID NO: 1 - DNA, 803 bp, Zea mays
cccccgcttttaccgaaaagccaacctgcaggaaaaatattcgatctccctgacgataaaaagaagcattctcacaatagcaactccacatgtttagtttcagtaattaattcatatggttgcttacttattattcacagtgcagaatgtgtaggatgggttcaggcttgtggtaaacttcaaaaaactagccctgcatcacaacttgcctaaatagcttagagtaactcttatggaaggtttacatagcaaccgttaaactatagacataactaacgtgtacagtcatcagtatacacacgagtacatgactaattgcttgtgttgctgtttacatacctcaaaacacagactaactgaaattattggttaagcttaatagaaggtaacggcctaatgaccagtaattaaatacatatctagcctaattttactgatagatcaagctggatgaatggatgatggcatgaaaggaaagtacgtacctgaagccaaggagatggaattccatagtaagtgtactcttgtggaatatcttgatttcttgctaacctctccaatattctcacacatttaggaagacaattccaatatgcttcaacattgttggatactagtgaaacaaaaagactcgtgacagatgtcagcacacctaaatcacgctcatctagaagttgtgccattcggtcagccctgatatacagaattcgattagcctattgattaagtagagtataatactataatagaatgttttctataattttactaccagccatcaatattcacaacatcaggattct

SEQ ID NO: 2 - DNA, 255 bp, Zea mays
cttgcatgcctgcagaggacctcggagaccagccccaccgcaactttcatgatgctcaggcccagcctctcgtggtagcatttcttcaggtctggggtgagctgcgccaggacggaccagccaaagagaaaggaggtaggcagtcagcgtctgctcgttctgctgcatcgactggatgaacagaacactcgactcgaggatcttttacaggctactaataaaagaaagaagagcaggtaaaaacctaccagtgtc

SEQ ID NO: 3 - DNA, 255 bp, Zea mays
cttgcatgcctgcagcgacaataccggcacgctcaccctcaacaagctcagcgtcgaccgcaccctcgttgagatcttcgccgcgggcgttacgaaggacgacgttatcttgttcgccgccagggcgtctagggtggagaaccaggacgccatcgacgccgccatggtcggtatgctgggtgacccgaaggaggccagggacggcatcgaggaggtgcacttcttcccgttcaaccccgtcgacaagcgaacggc

SEQ ID NO: 4 - DNA, 255 bp, Zea mays
  misc_feature (9)..(9): n is a, c, g, or t
ggtaccctnttggnccggccggggcaggcgtggtcgcgtgtagtcagctcgccttgcccgctctaatcggtcaccactgacctccgcccatggcgaccatgtctgcagccctcgccatctccttaccgtcgctaactcgcgttgccgtcgcaagtacgccctctgcttcctcgcgcatcaagagagccacacaattcaggtgctgcgcggattcttcttcccaggagcaggagacctccggcgcccctcccgccc

SEQ ID NO: 5 - DNA, 375 bp, Zea mays
  misc_feature (366)..(369): n is a, c, g, or t
gatgagcatagatgaaaacggattcatgctggagttaagggcttatcttcagaaagaggtcgccttgcacaccgataaatggtctgaagagcagattcggctcctggtttctgaggggagagcgctgaagaagctggttgaagcttgttgtgaaattccggttgtcgctgagcctgagaaggcgccggtcgcgaaggatgccagcacgaagaagaggaagatgccagatgggatctgtgaagggatggcgatgctgcagaacggtctcaaggtcatgcgaggcgcctttggtgaaggggactttgctgatctgaaggataggtttgctgcacacttgtctcgcctcgaggatgcagtttcccagannnntagttt

SEQ ID NO: 6 - DNA, 807 bp, Zea mays
  misc_feature (5)..(5): n is a, c, g, or t
tgaanagcangaaaggatcttgggtcatcgcatngcaccgcacgcanattatgctttcgggcaataattcaggcgctagtgggatcaatgatcggggtcaggccggccccgccattggaaagacgagagagattccnaaagccccatctccccgactcatccctcctcctgcctgagtcgacagacgccgtctcgcgcgctagctagccaggttctaacagctagcgcaaganngtcaacacncgcgcgcatcacgcatnccacgagctagctttcaggttatgtgtgatgtgcgtcgtctgcatgccgtcggtcgncntcgttcccttgtggcanagcnngcancnccatcncccncncgcgcaccgggcaccggncgcngtggcggtgcaccagcaccacacggtgcngatggacggccgatcagtgcgttcggcgtacgacgttggtccgcgcctctagttccatttcccaagcgcgnganggacgtctgcgtgtctccgtgcctgcgcgtcntcgtccccggcgtttctgtcaanaacgtcccctngcttnggngcnaactctgngncnttnctgccgttggtggtgtggcggaagatcaacggcggcgttggcnctggtgcnactcgtacnacttaaaattgtggatncggaacccggaatngaatgnagnnnngtnnctacactctnataaacccngnctactnctnaacaaaggcatncgagcggggaggggctggttttaccgaaggagctcactgtnatcaacgaagccacttgagtgacctcagagntccatccgtc

SEQ ID NO: 7 - DNA, 184 bp, Zea mays
  misc_feature (54)..(54): n is a, c, g, or t
cgctttacgttggatagtgacaactacatcaaggcaatgcgtctatttgtgggngagcctgtctggacaccatacaatcgtccgcatgaccatctcccggctaggaaggagtacgtcgagaaaatcatcaaccgaggtggaaaccaagccgtcgaagctcgttgagcgtgtccactctacatgt

SEQ ID NO: 8 - DNA, 613 bp, Zea mays
atcctgaatgacacccaactatagggaggtgtttggtttgatgaatcactctatgtaaaatgaagtgatgcatcatgggtcttgggtccatttctcaaatttggtgggatgactccatttcacatattagtactaaacaactaactatgatgaatgaggtggtgatggattaagtcactccatttcacaaaccaaacaaaaaagtgaggagagagaagatgatagactacatcattcctcaaaccaaacactccatagttgttttcttccattctttttctttgaatggtgaatttgtgaaactgtaatgtgaaactgtaattaacataaattcataacctcctattgtcttccttttgaagaaaacgcctctgttatccattgatttttttttacatgtctttcatggccgctaaggaaatattttgtcagcattgtctttatatttagtttctgctgcaggtgttctcgggggagggagtattcctgctgcaggacgacagctggacggaattttggaatacaccggcagagtagctgaaataacggctaggaatttctgagaaatttcatgttagggaaccaacgatgctgtttaatagaattggctgta

SEQ ID NO: 9 - DNA, 353 bp, Zea mays
tgagcctcatcatattgcagctggtgctatctatttggctgctaaatttctcaaagtgaatctcccatctgatggtgataagatttggtggcaggattttgatgtcacgccacgacaacttgaaggttggtccttatttgcgaagttagtgagaaatgtttccactcgctctactttgtctatttgttctgcatatgttgtgattgttattgattgaattggcaccatgtgtttggtgtgtcaaattgaattttgttcctgtgaaagtccaggttttgaaggagtgaataaacataatagatacttaacttgataactttgtgttttctttctttactgctagcagttcttga

SEQ ID NO: 10 - DNA, 607 bp, Zea mays
aggatctcgttccacaggaggtgtactcctgatgagtgtctgtttttttataccctctttcccgctttaatgaaaggcagagctcctgccatttttcaaaaaaaaaatgtgcaaagttccagacagttttaaggtaaattccaatcatgtacaagggcttcagactcagtcagatgccgatgtacaaacatgttacattcgtgtgctgctgtgctttttttttaggaaagccatacgacgcactttattgattatcaaacatgttacatcgtttaccagtctgaagaataacaccagagggttcatcgaccccaaatacagtttcctttcaagagagagctactctgctagttcatgtgccacctgtgcgtttgtaaattggacacagcattctgggagcagtgcacggcatcctcgtgaaaaatgagaagaaaaaaaaaggatatttcttcactgcctccgtctcctttcatctccggtatacgtatagctggacaagacacacatctatacagatcgcatcactggtaaacttgcacagagtaaatgattacacgtccagctctttatgcggctacagctagaggtctttggctggtctttattc

SEQ ID NO: 11 - DNA, 886 bp, Zea mays
tgtagagtttgcagaggcactcacctccaaaatgcctggcaatctcaaggtaaaagtactgtctgtctgggtgcagtcataacacgcaccatttcatttatgtttttggatcctgcaggttgcgtattttgtgaattctgggactgaagcgaatgagttggcaatgttgatggcccggctgtatagtgggaatctcagtatggttgcgctcagaaatgcatatcatggcggaagtgccggtacgattggattgactggtttgcagacgtggaaatacccaattcctcaggtatgtgtacagtgtaatcaagtattgcgctttcatcaaattatttgactggttatgttttcagagtcacatatcatatgtttgctgcagtagattcttagttatcaataattatctctccgttctacccagcaaaaatgtatccatttctttattacatctatcatagcctcataagaatttttgcagggtgaaatacatcatgtcatgaaccctgatccttatcgggggactttcgggtctgatgctgcagcttatgctaaggaagtcgaagaacacataacttatggaagttcaggaagggttgcaggcttcattgcagaaacattccaagtataaactttgaacagaccatttataaaatgctagaactaattgaaataatatgtatcttttgtttataaacccaattcaaaataacttatccttgtcgcaatcttgttgatacaccatctctgctgtagggtgtgggaggtgctgttaactagctcctggatacctaaagttagcttatgacattgtgcgcaaggctggtggcgtttgtattgctgatgaagtccagagggttttggtcgtactggcagtcattactgggga

SEQ ID NO: 12 - DNA, 333 bp, Zea mays
tgagtgaccatacaaaaatcatgcaatgaactctacaagccagtatgtcactggcatttcttggcttcatctggtagatagcagttcttatcctcactgtcgggtggtggcggcacaccaaatacccagtccagcacattcaggtacttggaccggaagacttctctttcctgcgcatagcagtgcataatgatagaggtagctatgctgaacacaaccacatcagctcgctttatctgaagtattggagggcagagtcctgcatcggtcatccatgtgaaaaaactctcgatagctctggcaagacagtacagggatatctcaatcctcctg

SEQ ID NO: 13 - DNA, 255 bp, Zea mays
ccacgcgtccggaaaggaccattttctttgtgaagatgaagcatgcttggcaaagaagtttgttgtttttctgagtgaagcagagctgaagaggcataatgctatggagcacggtgggcgaatgtctcgttcccagaggaatgctgcacttcagatacctaccagttttatatatcgcagaaatgagcaagcacaacggcgtggcagaggtagggggcgtaatgctctccatgaccgatttgacagtcatatctc

SEQ ID NO: 14 - DNA, 546 bp, Zea mays
gctgctgttgtcgacatgtcgcggaactgcccggagctgaaggtattccggttatgtataatgggacggcaccagccggaccatgcgactggggagcctatggatgaaggattcggtgccatcgttcagaactgcagcaagcttaccaggctatccacgtccgggcaacttactgaccgggcatttgagtacattggcaggcatggcaagtccctgcggacgctctccgtagcattcgctggaaacagtgatgtggcgctgcaatacatcctccagggctgtccaaagctagagaagcttgagataagggactgccctttcggtgacgctggcctcttcgccgggatgcaccatttctacaacatgcggttcgtctggatgtcaggctgcaacctgaccctgcaagggtgcaaggaggtggctcaggtgctgccacggatggtggtggagttgataaatggccagtctgatgagaacgaaaggaatgaaagcgtggacatcttatacatgtatcggtcactcgatggtccaagggaggatgttcca

SEQ ID NO: 15 - DNA, 425 bp, Zea mays
ttcaggtacttgctgcaagatgccagtatgatctctgatgcacctttgcagtgtgctcgcagtgcaccctctgggagctggattaccactcccattctcttcttggcagagttgaacggctccacctttacaagagtgcttgctttgcgcaccgccaaaaagtctccaccaagtgacagcccgaactctagaatcgctgtctcagtaggtgtccccagtatttcacgtttgccatcctggttcaagacaacgtctcccccagtgttgttgaatatggactgggagagaatcgccatgacagagtctgggagttcagagaacaggttcttgatatccgaaacaccatccacttctttgatcttaccacagatgcaggctttgacaacggtcatgtggttcgttgtgagagtgcctgtcttgtcaca

SEQ ID NO: 16 - DNA, 385 bp, Zea mays
tttgcccttcggtctgtatggctcgatccccttatgattgggcatggcggccgggcgtcgtctggcttcttctggctcaacgcagcggagatgcagattgacgtgcttgcaccaggttgggatggggtcactgaccatgagaacgggcggatcgacacactgtggatggctgaggctggtgtcatcgatgcattcttctttgttggttctgagcctaaggatgtgatcaagcagtacataagtgtcacaggcacaccttcaatgccacaacagtttgctactgcgtatcaccaatgccggtggaactaccgggatgaggcagatgttgacggtgtagatgctggttttgatgagcatgatattccatatgatgtgctctgggctc

SEQ ID NO: 17 - DNA, 553 bp, Zea mays
tataaatgcttaaacttaacctggtaaattcatctacaggctctttcaaatggcaaacccactgttgttgagttttatgcaaactgatgtgaagtctgcagggaactagctccagatatctacaaagttgaacaacaatacaagtaatttgtttacacaccacttttcacatttctgaactttaagccttgattgagaataacaattgtgattcatttaataaaatcaatgcttgtcgatgctttgttgcgttgcctatcctattaaccacgttgattgaaccttgctatgggcatgacagttgggatccaagttgctgcacgacagacagaaccagcgaacaccacggacaacgacgacgacgaaccagagcaggtccgcttctcgctcttgggtggcaagaactgcagcaacgttggtgtcacgaccgccattaggcccatgaacaacgccaggctgaggtccagggatggaggcaacgccacgcccacgaccatcacccacagtgactctgcatcggggagcatgaggacgatttgtacgccatcacaat

SEQ ID NO: 18 - DNA, 765 bp, Zea mays
aagagacgtttcatccgcaagcgtaaaagcaagatgctagtgacacttactgaatgcagtaatgcatccaaaatccttttaaactctgacaagatatccatctactagtgtacacaatacacgacagtatgtgcatgtacaatgtctggtccccatttggccaagatcattcatactggtagtattcatcaagcatcccaagaagctaaaatcacttgagattcttggaagatgcgagtgcggaagaagaataaacaaacagcaaggtttgggtatgtgctccgcaatcatggagccggcacttacattttacaagtccttgaaaaggcaaagctctacagttttagctcgcatcctttcagcattagcatgcgtgtcggcagtgctcctcctccctcgacgccaattattcagaaaactatgcgtcgggtaaaccgaccatcctttccactgttccagggcacgctcgggatcggtgaaaccatcataccccaggacttcaccaatggatcccatgatgtaaaatgccagtggagcagtcacccgctggacaaattgttctttaatgttctctgatgaacactctgcgactgaactcttggaatcttctcgagtacagctttgggtctacggcagaaatgcaaccagggttgacctgaaaggacttgtatgcatgctcgagcttcttgttatgtcgtagtcttgtagaatgtcaattattccaaagaagagatagacatcctggaatccgccacttgatgtggt

SEQ ID NO: 19 - DNA, 429 bp, Zea mays
gatgaagctttggactgtgattgtgtgtcctttgtcttcattgaatagtatctaagtgcaatctccactgtcatttggtatccaggtgacatccatggtcaatacagtgatcttctaaggctttttgaatatggaggctttcctcctgaagccaattatctattcttaggtgattatgttgatcgaggcaaacaaagtttggagactatatgccttcttcttgcttacaaaatcaagtaccctgagaacttttttcttctgaggggcaatcatgaatgcgcctcaataaacagaatatatggattctatgatgaatgcaagcgtcgatttaatgtgcggctatggaaggtcttcacagaatgttttaacactctacctgtggctgctcttatcgatgataaaatattatgtatgcacggtggactctca

SEQ ID NO: 20 - DNA, 255 bp, Zea mays
gagtctagatcctcggggcatagatggaataacatacatcacgcggttttaagtagaaacaggaggcttcccaactctctcgcctccaaacccccactgtttaaatggcgtggtcaggaggaggaatcagaggccggttgttctggctgtgcttcagcagcgcgcttgacggacgcgcacttgatcaagtggttgtagcttcccttctccttgaaaagttgggagaaatggtgtttgcagtagaggatgccctcc

SEQ ID NO: 21 - DNA, 812 bp, Zea mays
  misc_feature (710)..(712): n is a, c, g, or t
gtgctcagtggtcagtgcgaggtgtccacgagtcaattaaagttggtttcttattcgtacaaagcatgcaactcttgaacagtttaataactacatatttgtcggatgatcctttcttctctcttagctttggcttgtcttgggagtcttcctaatggccacgagcctcagaatgtatgccacgtgccagcagcttgctcatgctgcagctgccaacagttttctcggccacactgagctgcgtgtgcatgtaccgccgtccattacccttgctacgagaggccggttacagagccttagacttcaactggctcttcttgatcgtgaatttgacgatttaggtgtgccaatacattccattcgactcatttgatggtttatagtgtttcctgaaaacttagtgccatggaaattttttgtttcagattatgacactctgagagcattggatgctgacaatagcccacatgctccatctatgagtgaagaagaaataaattctcttcctgtcttcaaatataaagttcaggcacaacagaggcatccccctgcccgaaaaaggtaaagccgaattgtcctttgggttgatttgttcatatcaccatatcgagttggttccagtatttcccatcaacacatgaccaacatttaattctgaaatggagacatgaagtaattttttctaatgtttgttttccaatagtgatggnnnatctnnnntatcagtttcttcaactgggtccggcaatnnnnnnnntgataaatactgacttgnnnnnnnnnnnnnnnnnttgaaaagtatcttattatca

SEQ ID NO: 22 - DNA, 543 bp, Zea mays
gcaggtcatacttttgtccaatatccaattgaaatgtttccattcaatatacataccttttgcatttcagctcattataattccttgcatatgcctctgcagacttatgagcgggggtacatagagatgtggctggaggctggccatgatacctgtccgaagacacaacagaagcttgccaataaatccctgaccccaaattatgtcttacgtagcctcataactcaatggtgcgaggcaaatggtattgagcctccaaaacgccctgctcaacttagggatgcaccactgtcatgtagtgctgctgagcatagcaatgttcttgagcttcttcagaaattgtcttcccaaaaccttgaagatcaacgtggctctgctggtatgcttcgtcaacttgccaagcgcagtgctgaaaatcgtgcttgtattggagatgctggtgctattcctattcttgttagtctgctatcaaccactgatgttagcacccaggaacatgtggttactgctctcttgaatctttccatctacgaagagaacaag

SEQ ID NO: 23 - DNA, 789 bp, Zea mays
ttgcatgcctgcagcgacagattacacggcctcacagacacaggatggttgccttggttcgaacatcaaaacaaacaaacaaacaaatgcagccttcaatcttatattacgcctatttacaaacaccgtgcattaacgaggaatcgtgcacggatccgatccatctctaatatcggaaactggggttcagctcgatggcctcgttgaatatcagctgcttcatctggtcttctgtcagagcctgccgctcgaagtcgaacgagaacggctccgtgcagacgggctcgtcgtcgacgtcgtgtagccgctccagatacgggtgcgccagcgcctccgcaactgcatggcagaacagatcccggtgactgtgagaagtgcgaggacaaaacctgccatggtgccattcgtgaactcttacagtcactgtatatctatacagattacaggcagcccatactacacatcatatatatgctagccttgtaatctagaaccataaaaacccaaaaaaaaaaaataaagtctgctaatctgaaaccaatagggggttgattggttgattctctgaagtaaactccgctggtctggacagctgcctgcctgccagtagaggaatctaatcttcaagattatctatagcctttgctgttttttttccatcttgagacatgtttattttgtacgcacatagccgtcatcgtcacttcggcaaaggcgatgtagaggaggctacgaagccgaggatgggtcaaaacagcagcagcgcttcaaccatcactccatcagtac

SEQ ID NO: 24 - DNA, 255 bp, Zea mays
cgaatggagaccagatagtttctatgagaagattcagaacaatcgccagcttgggctgcacacactttgcctgcttggtttgtgactcttgatcgacctttctggattcttcgttatcaagaattaggcaattgatctaatatttgtagccataggtaatgtgtcttatagtttaaccattgtacagggtatactgaacttaagtagacactgattattgcaagcaaatacaatgaatgttcaaactattagact

SEQ ID NO: 25 - DNA, 467 bp, Zea mays
acttcaaggactgatttgcgtttccagagccatgcggtgcttgccctccaggaggctgctgaggcataccttgttggcctgtttgaggacaccaacctgtgcgccatccatgctaagcgtgtgaccatcatgcccaaggacattcagctggcaaggaggatccgcggcgagagggcctaatcgccacctcaaacatcgtgacaaaaacatgaagtcctggggttattgttaatctggtgccattgtaaggacatatgagtagggtttgttttgtggatcgcaagtttctcttctgctactgttgctgctacctttgctggtgttattgctagggtcgaatacttaagttaactatgcgctgacagtctggactagcagtgttatgtgcgctgctggaccttgcgcgcaatgctgtggccgatttgttcgttaatgtcaagaattgtgttacagtgcgtgtatatatg

SEQ ID NO: 26 - DNA, 605 bp, Zea mays
attacacgcactgtaacacaattcttgacattaacgaacaaatcggccacagcattgcgcgcaaggtccagcagcgcacataacactgctagtccagactgtcagcgcatagttaacttaagtattcgaccctagcaataacaccagcaaaggtagcagcaacagtagcagaagagaaacttgcgatccacaaaacaaaccctactcatatgtccttacaatggcaccagattaacaataaccccaggacttcatgtttttgtcacgatgtttgaggtggcgattaggccctctcgccgcggatcctccttgccagctgaatgtcctttggcatgatggtcacacgcttagcatggatggcgcacaggttggtgtcctcaaacaggccaacaaggtatgcctcagcagcctcctggagggcaagcaccgcatggctctggaaacgcaaatcagtctgcaaaacaatatgttccatcaacaacttgacacaatgatataaatgtacttctagaagttgaattgagatatgtgtatactttgaagtcctgtgcaatttccctgacgagcctctggaagggcagcttcctgatcagcagctcagtgtc

SEQ ID NO: 27 - DNA, 719 bp, Zea mays
aaaaggaagattccaaacttgagccgtgagtatagaacagctattttcaccaaactgccaccgactgttgacaatagcacatcttcttcaggaaaaagggaacctgaaaaataagatgttttaggaaagggatactctcgtggtgtcatttttctttctggttgtgtccaaattcataacattgctatgttgtctttgaatagaccatctcttagcttaaatccatttgggcatcatattgtgttggtgcatttcaatctttatttgttttttctttccagttggtggtcatgcactgagtggacccactcttttttggagttagagttgaaaaacgagttaatgggacaggaagaatcaactttagaagggaatcccactttctagtagtgctcatatgcaaaaataccttccaagacaacacatattgtcaagcaagtggaagtaagatataaatagagcaaggctaccaaggcagcaaggaaaattccccccctgttggtattctggtcccttatgagttcatatgcttacaattacgaataaagaaaaactctagtcaagtttaactctgccaaaaaaaaaactctatgaaaatcatcctccagtggcccatgcaaaaaggattccccgacaccaatagggacaaagtcaactatgccttcttttgtccatttgacttatgtaggttgcagattgggaaagaacctgctcttttc

SEQ ID NO: 28 - DNA, 806 bp, Zea mays
cttctgatacttgcggatctcacttataaattacattcatgtcagtagtgcagcataacagcatattcaacggaagaatccaatgatgttctactcaccggagggcaacagttccagggcggtagcggtgaggcttcttcactccaccagtggtgggggcagacttacgggcagcctatatatgaatagccaaaagggcaaattcagtcatatcttgatccaatatcaggggtcaaatcaaaaaaaacaaattaaggacaatatggtaccattatctaaagaaccttaccttggtggcaagctgcttcctgggagccttccctcccgtggacttgcgagcagtctgcttggtacgagccatctgcactaatcaagggggcaaaacatcactatgtattcaggggccccaaaatctaaccaattacagccagtaaccttctgttctcatatcagcaaatgcacagatttcttaatttctgctgtgtaaaacaaactactaataatactggatacaataactctcagctataatcatttccaatcggtcaccttggttatcattatttcattatttataaatgaaatataagatagttcaggtacaatagaacaacaagcattttgttcaaacatatttctaatactatataatagaatttgaatatatttgttgactgaacaactagcactataactggaccgccctatagaacagtactccaaataacaccatgactagtaacaaatgtttaagaaacgaaaaccattaaatcatactcaacgacctgattaataaatgttcgatt

SEQ ID NO: 29 - DNA, 498 bp, Zea mays
catgtcgcggtagaaggactcctgtaccccctgctgcgtcgccagcctcgccctctgcaagaaagaaaggtcgaagtccgtttcattgtggtgggagacagagagaagctaactaaggcagggcaaccgagcgaggggagagacgaaccgagtggaacatgccgttggagagcgccatgacgcggttcttctcgtccagcgcgttggggaagggcgcggagccgtcgacgacggtggaggtgggcagccacggctggctcatccaccagtgcagcagccatggcgcgtagtaggcgacccggagcgaccactggtcgccgaccggctgcctcccgtagagctgcttggccagcccccgcggcagcccgcgccagcggtagttgatgactggcgccatcatggccagccccgccagcctgtgcgggatgcacctgacggcggcccagccggcgtgggaaccgagggaggagcagacgaggtggaacctgtcacccagac

SEQ ID NO: 30 - DNA, 155 bp, Zea mays
  misc_feature (101)..(101): n is a, c, g, or t
cttacattttcttgctgatcacagctagtagggagcgagagttaccttgaaatcaaggcagtacgtacgagtacgatcgacaatgtcgacggtcacttctngcacctttctgagcctcgggtccccagtgcggccggcagtctcctccgcgagac

SEQ ID NO: 31 - DNA, 255 bp, Zea mays
  misc_feature (179)..(190): n is a, c, g, or t
gaatcggaagagtagcaaatatctcatgcccaagttcccaactgcccactacctctcgttcccgtctccttcctctattcgtgttgtccgttcgctgccgctgtcgcctggttcgcatcgcatacagcgcctcagtccagatcgatcctccctcttcccagatcgggtctagttggacnnnnnnnnnnnnccggccgggatctgtcatattcacgccgcgtgtgctctctctttctgtctccctctctatctgcg

SEQ ID NO: 32 - DNA, 659 bp, Zea mays
tagcgacggaacggaaactcggaaaggcaccccatgcgcgttggatggaggagccctggagagtggagatggtgtttataaatccgctcctcctgccaccccaaggtttcagaaacccaccaccgcaacacctgcgctccgcggcctgctctctctttcaaacagcttcgtgtgtcgttgcgccgcgcatagttccagcgccagctcggcactgaagaatctggtgaccggccgggatcgagagccaagctcgagcaggaacggagagcgcgcggatatatatatagctagaatgatgatgatgatggacaggcagctcgtcaggcaatgcgacatggaggtcatgaggatggccatgctcaagcacgaagagaccttccggcagcaggtaaagcgcgcgccgacaaacttcggcttcttcttcttctttctcctgctgtccttcacaccgtcgctacttgtctaatatatttcacacatcatcctgcaaaggaagtgtatgaagcaagtatgaagtagaaactgctgctgctactgatcgatcgtggaatggattatcgccattgttcttggcaactaaccgtcttatcgtctgatcaggtccacgagctgcaccgcctgtaccgcatacagagggagctgatgagcgacctaataac

SEQ ID NO: 33 - DNA, 827 bp, Zea mays
tcttgttctcgactcactggataaggtagatttcctcatgcaacaaagacataagactggcatgccctttaattcattattcttcctatggttgcaacttgcaacaggcctccttacatagggacttctgcagatgattattttactgatgccatgcaatttcttgtgtctcttagtttgacccagcaaaactgaagaaacttcgcccaagtttcaaggagaatggtggtaccgttacagctggaaatgcttctagtataaggtaacttcttggcatttacgttattattacttagcccttttaattttatcttcagctgtattataattcttgtgatttactatggtgctagtgaacttctttctttacattgtgctggtactttgttatttttgttactgccctgcgcataccatatttgatttgtgtttgttcttatgaattgacactcctattatgttttaggtgaattagttaaattattttaagttttcttcctataaatgattattatcacttcagaaagtaaaaaaagtgtttgtaacgtcatattcccgttcaaaagaaaatcagcctatgtgtataatttcttcttggttgtcttcaaaattctttcatctcactttgcacttgctattttttccattggctggtaccgttcagtgtgtactgcctactactgcttgtgtagctacaatcagtcatatctgatttgcttttatatggatcagtgatggggcggctgcattagttttggtgagtgggcagaaggctcaagagcttggcctgcaagtccttgcaaggatcagaggatatgcagatgcag

SEQ ID NO: 34 - DNA, 493 bp, Zea mays
attgccttggtaaaggtggtcaattagcggctgcttataagcttttctgtgagatgattgaaaatggctgcacacctaaccttgttacttacaatattatgattgcgctccaagccaaagcaagaaattatgagaatgttgttaagctgtacaaagacatgcaggttgctgggtttcgtcctgacaaaataacttacagcatcgttatggaagttcttggtcattgcggccatctggatgaggctgaggcggtttttattgagatgaggcgtgattgggctcctgatgagccagtatatggacttttggtcgatctctggggcaaagctggcaatgttgacaaagcgctaggatggtaccaagcgatgcttcaggatggtttgcagcctaacgtgccgacctgcaattcgctcttgagtgctttcctgaagatgaacagatttcaagatgcctacatcgtgctccagaacatgcttgctcgggggcttgttcc

SEQ ID NO: 35 - DNA, 357 bp, Zea mays
caactgtagtctacagtctgaattgaattatttctgtctctgttatgaagacatgctcaagttcatgtatttcacatacctgtttatgtagcatctctaacaaactactggaatttatacaaatggtattttattgttgaatattaaatgtttttctatctgtcagctattctctatgatgaatacatacatgtctatgtgatgaagacatgctgcagttgtgttagccaactgaacgcgaggtacggtgtccagtgttgtacgtagaacgggggtttaggctaactccaaatccccaacaataccatctatttctagggactcattcgaagaacggagcatatgaagtattgtgcg

SEQ ID NO: 36 - DNA, 729 bp, Zea mays
gagatggtgcagcgcctcacgtaccggaagcgtcacagctacgctacgaagtccaaccagacccgagtcgtcaagaccccaggttcgccccgtctgttcattcctgctcgatatgggaggggtctttggtttctttatctgtgttgtgttcttggttgtattaattggttcgctggattcttgcaggcgggaagcttgtgtaccagtacaccaagaagagggcgagcgggcccaagtgccccgtcaccgggaagaaaatccagggggtgagtgattgattgttagcgcagctctactcttcttctgtctcggttgtgtgattcgttggtccttggtaccgaggtggggaactggtagcgtgtttgtgctatccatattgaagtaacagattgaagtaattggtgtgaattagtagatcttaattagaagggcaagcctggtccagttgtaagaactgtctgagtcaccagcgggtttgaacccttgcttcggtttatcccttccccagatcgcacatgagttgagcctccggccctccctttattttagtagattttaattgattgcttacatggtttactgtgttttgccctgtgatgccacattgtgcttgtataggtctattggacgatgtctgtagttaaatgggcctttatatctagctctcgtctaattttttccttgcgtaaatggctgtgtcatgtgaaattgaagtacttggtacctaca

SEQ ID NO: 37 - DNA, 832 bp, Zea mays
tgcctgttgccggccgggtttgccatggggattggtcgaagattaaaaataagtacagttcttttgatgacggacaactgcttcttttttgcttttcctcagcatatattattgctctactgcatgatacgcttaaaataccactggatcataaaagtcgtgatgttgtaaaccaaatccatggcgtgcctgttgactgggcattgggtgctttcattgtgcagacgacgatgaacagaacagagtactcagattcatcagtttcatatctaaacagctatgactctcctggcttggttccgcttctgttcattattgtggttgtatttacagcattgtccattctaaggtggagaaaacctcgtctgaagacgatctacgatatggagaaaggcaggtacatcataacaagggtcagccaatgagtcgcgcacatcagcgttgggagtgggtggactttctgcagcggagcactattgaccatgtatgtagattttcctaaattgatttcttacttagtggccaactacctccagcatgttgccttgcggagtcaacttgttgagtttgagcagcaacccctgtatatgatagaaggatgcgggttgccgtggcatttgtgcatgttcagtgttgtatgtaatgttgctaaactgttgttgatgtaattagaattttgtgcatgggcatccatccgatgatgtaatcctgagataaccatgtgtctaatgatcgattgaagttctctgccatggatcatggatggcagggcagctgtatctttgtttcatgtcaaatcactgatgttatgtaatcctaagatattatttct

SEQ ID NO: 38 - DNA, 255 bp, Zea mays
  misc_feature (173)..(190): n is a, c, g, or t
cttctcgactccatagcgcaccacaccagcgcggcgacagccagcccgcgaggagaaaaactagaagaggaggaacagggagagccatacatactcactggttggattcggtccaatccaatccagccggccgcggcgtgaccagccgatccagagggcgccgaatcgatctnnnnnnnnnnnnnnnnnntgcggagggctaccgctggggagcccggtcgccgcgggggatgcgcatgagggcgtccggcccgc

SEQ ID NO: 39 - DNA, 475 bp, Zea mays
catgcgagttcctctgcccgtttctcagtagaaattgaggtctgtgtacccatagtgatcgatctccgtgtacccatacgagtacggcagcagttcctctatgtccagctgggagctccaagaactcatcttctggatctggctgtggttgatcgattccacctctgcaatgaacatgctgtagacaggtctgtggtccgagaagcgcgactctccccggacgtaggagagctggctgagcccccttccgtaccacaagatgcggtcgcacctggaatggataaacgggttgccaatttttccttagctaactaactgtgacaaaatttgacaaatgaactggcgaattcaagaattgttattgcagttacgtatatatatataggtaccatgctggagtccgcttcttctccttctggttcaggtcttctcccgcgtatttgtcggagttgttggagtatttataggtcggtgg

SEQ ID NO: 40 - DNA, 382 bp, Zea mays
cacatcttcaacaccaggtggaggttgctgcctctgcggttcggaaggcagaggaggagcgctccatcctcgctcgtcaaatggaggaaaccaggaagacagtgtcgcgtctttgcttggagtggatggccaaggagatggaaggtatgggatcgaaaactgagagggagaaccgtgagcgtaggattgttgaggaagagaatcgaaggcgagaggaagaggatcagaggcagggggagttccgtgagcagcgagggaggaacaaggagattcgtgagggggagttccgtgaacaacaagggggaaattgagagtatagagcagcggatttgccaaaaatgtcattcccaaaatttcagggaactaaagcagggatatggat

SEQ ID NO: 41 - DNA, 798 bp, Zea mays
  misc_feature (777)..(780): n is a, c, g, or t
cttccaccttcgggtccgggttcaccccttccatgtccttcgtatcaacaagatgctttcgtgtgctggggctgataggctccagactggaatgaggggtgcctttggcaagcctcagggcacctgtgctagggtggacattggtcaggtcctcctttctgttcgatgcaaggacaacaatgctgcacatgccagtgaagctctgcgtcgcgccaagttcaagttccctggccgccaaaagattattgagagcagaaagtggtgagtgcatgatgaattcattgctgccatgaaaccaaaatgccatagcctaatagcttgcataagttattttttattattgttttttgatttgcactgtcttgtccaggggcttcaccaagttcagccgtgccgactacctgaagtacaagagtgagggtagaattgttcctgatggtgtcaacgcaaaggtacagctatcacatatcttgctacatttagatatatctctgaattagttttgggatagtaatgtaatttccattgatacgcatattttgaaatggagctattcttacaatgtttgctccgtcttctcttcggcagtatattaaccagttcataatctaattttaacttctcctgacttattacagctgctcggtaaccatggaagacttgagaagcgtgctcctgggaaggctttcctcgaggccgttgcttaagtccaatctacagttggaatgcgaatccagacgttttggtttagcctatcttattttgctttgtgcaacnnnnattttaagtgtatggggt

SEQ ID NO: 42 - DNA, 391 bp, Zea mays
  misc_feature (381)..(387): n is a, c, g, or t
ctgaactacaaaatgaaagaagcaaatatcatgaaacaggcgctgtgaataacactagtgctgggtcactcagatcacaagcaaggaggtattcgactgcagcgagttttattggtgtccaaggaccaaagaaagaagtcacttctgagttaccgagtctggataagttcttggtgaagcatctttcaaaacttgaaagagaagttcaggaagcaagagaagcaagcagaaaatccacattagtaaagtctgtcacacaaggtgcccacatccaatttacaggcagcaatgcaaagacaccagaatcaacttcagaactcagcagcattctggtgaagcatgtgtctaagcttgagaaggaaatcctggaagcaaagaagnnnnnnnaaag

SEQ ID NO: 43 - DNA, 753 bp, Zea mays
  misc_feature (76)..(76): n is a, c, g, or t
atccttgtctaccagaatgatcccatttctaaattttgtgcgccctttactttttgtttcctaggatgttcatcanatctgcccaagaagacaacaagaaggtgagaaagatttgttgcatttcaacaatgaaatctgttcttgttccaaatgcctgctgcttgtttatgatgttgtctgatttgttatgttgtgatgcatattgcagtatcctnnnctccagttggagttcctggggagctacctttctgagctgagcttgctggattatggcttgattcgctccttgccgtcacttgttgcagcctcagccgtctttgttgcaaggctgaccctggatccacacacccatccctgggtataatctcgatctgtttgatctctatacattcctgtctcatcaaatacaaaccaccaactcattttgttatgaacgggttgcagagcaagaagatgcaaacgctaacaggctataagccatctgagttgaaggactgtgttgctgccatacataatttgcagctcaaccggacatgtcaatccatggtggcgatccgggagaaatacaggcaacaccgagtaagcttgggctttgacagctgtacatggttgttagattgtttgcataactctagtgtactaactgtggaaaaatctttgaacagttcaagggtgtatcagctttgctacctcctgttgagatccctgcatcatacttcaatacactgaaggagtnnnnnttccttgataatc

SEQ ID NO: 44 - DNA, 796 bp, Zea mays
atcttgcatgcctgcagagttctagagaaacataacacgtatgtatgggagagggagacgcctcaaaagttactaaaactaagttgtacagagcatctaacatgagatctttaagcttttgaaattgtgagaataacactggattttgctggtggtacttatggcacttagatatattctttactcctgtcgttacatgagtactgcttttgtctttgaaataatttccaaaatatataacaaaattagttttagccaatctctgttgcaagatttatcataatgcaatattaacttgattcactgtaagtttcttagctacattaaatttgtatcatctgtcaaacacatcaagtgctggaggtattcatttttcctttagacaaaacttgtcaaacacgttggttttgcttggacaaaatattgaggacatatatactttattttttttttcagtatcaacgaaggatttcctggaaaggcctctagcaggttctcaagtacagttatgcttgctggaaacaaatacgctcaacagttggtctcgtattgatcctggtacattccgggtccgtggagcaaactattttaggtaaatgtcattcttgcagtatcaggtcatacccatcatttatttatttgaaactcctggatgctttatgggagtgatcttagttttgaatgcacttttccagggataagaagaaagagcttgctccaaattatgctgcatattatccatttggagtcgatgtgtacttatcaccacagaaactcaatcatatatctcgatttg

SEQ ID NO: 45 - DNA, 795 bp, Zea mays
  misc_feature (145)..(145): n is a, c, g, or t
tgggatggacgatgctgatgacatatttatgcggtcctggacaggaactatcattggtcctcccaacgtatgctacagaaatacccataactagatacataatgcatttgttttgttattctcaaacctgagatgtttcactgcntttttcgcagactgttcacgagggacgaatctatcanttaaagctgttttgtgatacanattatncanataaaccaccgactgtccgattccagactcggatcaatatgacatgtgtgaaccaagaaactggattggtactcgtgctttttttttgtttcttatagctngcaacctaccaaggcaatttatgaatgatagtaggcctattacttaactagcaccttttctaggttgaaccaagtntatttcctatgcttggaaagtgggaaagggaacatacgatggaggacattctgaccagnttaaaaagggagatgtcaactccacagaatcgcaggctgtccaacctcatgaaggtattttttttctgttcatccctccagtaccttatatgctgacagtgctgtttaactgtagccacgcgaagtaatctgtnttgaatatccaaaactnttgcccagactagattgtaaagccaaaacattcccgttttttggaaaatgactanaaacacccatgttgaaatttttatnttgccnaaagctggcancagggccagatacaggaaccaaatgagcntgggcctcaacaatnttttgcctttccctgtaattaacattaattaggctctgttggttccttacagga

SEQ ID NO: 46 - DNA, 255 bp, Zea mays
ggttcaatatgtataggctcttacgcttcgcggataattacaaggtatagatattatctattagtcaaaataacagagagagaaaactagtatgtgtgcttacaagttgcaatttagtatataaacataattccagaaaagcaaaaaccagaaactgatatacaacaactagaaaaaaaactattttcagaagtaaccaattaaaattttgttcttgactatggtgctctgctaggtataatataaacatattat

SEQ ID NO: 47 - DNA, 255 bp, Zea mays
tgaaacaagtagaattattttttattgcatacatgaaatggagaccaccataatcagtaaccatctgtagaagaagtggtggtttctcatcttccgtatctttcaggaagagatgttttccacttctcccagcaatccaagacaaacgatacgccatcttttctaatttgttggaaccacgaagaagtggtatctgccagtgcataacagaaaccgaatcagtcaaatatgtggattcttcatggctatgcagcc

SEQ ID NO: 48 - DNA, 623 bp, Zea mays
agtccgctgccaacgccggcggcaacggctccgccttcgtgaaggtcagcatggacggcgcgccctacctgcgcaaggtggacctgaagatgtacaacagctacacggagctctccgttgctctgaagaagatgttcagcaccttcaccacatccggtaaatcacatttaccccgttggcgtaggaatctgcagagcatacagtttgcgaactcatagaagctgcatccatgatccatcttaccagggaacaacatgaatgagggaaaactggttgatccggtcagcggtgctgatgtggtcactacttacgaagacaaggatggcgactggatgctcgttggcgacgtcccgtgggagtatgttctgcattctagtgtgccatcatctaggatgtgatcaggcaaatgttcaggcttcagcttcttatatgctgccttgttgattgttgtccctgcaggatgtttgtcgagtcatgcaggcgtctgaggatcatgaagagttctgaagccataggtcttggttagtgtctccagataaaattatcttccatagttccattataagatttacatgtcaactctaaccgcctatgttttcttcatttttttaaaaaatacagca

SEQ ID NO: 49 - DNA, 680 bp, Zea mays
tctacattgaagtgcaattaagattataatattgtttatgtgatgttgttcctgttataattaacttattttctttcttcattttcatacaatccatgctaatttcctaatcatttccagaaccgtggcatacactaaccatacagtgcttcctgaagctctagagaagtggagcttggacataatgcagaaacttttacctcgacatgttgagataatagaaacaattgatgaagaggtattagatgtgcagaaagtgcttatgcaaatttacttcttgctctatgagttaacgtacacttttctctatacttgattgaaacagctgataaacaacatagtctcaaagtatggaaccacagatactgaactgttgaaaaagaagctgaaagagatgaggattctggataatgttgaccttccagcttccatttcccaactatttgttaaacccaaagacaaaaaggaatctcctgctaaatcaaagcaaaagttacttgttaaatctttggagactattgttgatgttgaggagaaaactgagttggaagaggaggcggaggttctatcagagatagaggaggaaaaacttgaatctgaagaagtagaggcagaagaagagagttctgaggatgagttagatccatttgtaaagtctgatcctaagttaccaagagttg

SEQ ID NO: 50 - DNA, 482 bp, Zea mays
catttctggtgggaaggctagtagtaaaagacagatccgcagtagggatgctttgccgactgaattggagttccacacgtccgtgtctgtgaaatatttactattatcagctttcctggctttcaaggagcccagctacgcttgatgcagctttgttcgattcaactggagggtcagatagccataggcgtaagagaaagtatgccacctaatctcatctcctgacttgccaacagtctaattatcttattagaatattagatgcttacatcgttgttccttgatgtaggagttctcaggtgttgacgaatatgaaggacaccatggccgaagagatgcttttgaaaggccctggcacatttcctcttgagaggctcttggctatattttagtgtatcacgtcagtgtcataagaagatctcggtgatgttgaatgtcctgatagtatgatgaatgaaagtcgaataactggtttgatgtca

SEQ ID NO: 51 - DNA, 407 bp, Zea mays
ttgcaatgaagatggttctgatccagaatatgagaaattggggtggaaaaagcttgaggattatgatccatctcagaaacgttggaattgtcttgctagcctagttgagggtggtggtctttcagaagatctggatgatgatactgatgagatgacaataaatgattactatgcaagcctgccatttgcagcactattctctgcctgcaaggtgtctgattttatttcagattcaaaagattttattgcctcaaaagttgagtgaatgttatactatcactttatctgtgataggctaaaggcttgaaagtttcttgtgtactgtgctactgctcggaaggcgacaacatgccagaatctttccagttagctgaagcaatatgcaaacttctaggtcatggccctga

SEQ ID NO: 52 - DNA, 672 bp, Zea mays
  misc_feature (94)..(94): n is a, c, g, or t
cccagaccattcttttccaagtgcatgacccatcatttcatggggcagtggtagggcagtagcgcaactttccgacaaaccaatatttcctgtntctctcaatttcacacttgcatgtagttgtagattatgcttaccatgaccaggtaacttcagagcaagatcaagaatctcttctgttctttgaagacaaagcttcatacggtcaccagatgacaatgaagtatgtattgtcccatgggaagaatcataaagctgctgcatggaacgatcatgaggtatggactgagaaatgtattgttgctgttgctcctgttgcctgagctgtagcatgttgttcaatagagaggattgaggttcagggactccaggaatcaggggttgcaagtgtagctctgatagcagatactgttgttggttgatgtttaactgaggatcatgatatattccaccgagaaacttttctggctgtgtgatttgngagttcaaacaagctaaagttggttgattctgtgttaaaacgtaatgctgttgagcatcaatacctatttgctgagaattatgcagattttggtgnacatttantatctcttgngtaanatctataccacacataccagcatgcagatttccagagtcancattatttgaccgaangncaatccagaattg

SEQ ID NO: 53 - DNA, 255 bp, Zea mays
  misc_feature (10)..(49): n is a, c, g, or t
tcctctcccnnnnnnnnnnnnnnnnnnnnnnnnnnnnnnnnnnnnnnnncgcacacacacnnnnnnnnnnnnnnnnnnnnnnnnnnnacgtcgtacgcggcagctagcagagcacgcacgatcgccgcagcgtacgtactagcacagaggacgacgaccgacgccatggcggccttcttnnnnnnnnnnnnnnnnnnnnnnnnnnnnnnnnnnntgatggtggcggccgtgctggcggcgtcgtgggccnnnnnn

SEQ ID NO: 54 - DNA, 394 bp, Zea mays
tgttcgagatgcccaccggcggcgccgccatcatgcgggagggccccaacctcttgaagctggcgcgcaaggagcagtgcctggcgctcggcaccaggttgcgctccaagtacaagatcacctaccagttctaccgcgtcttccccaacggcgaggtgcagtacctccaccccaaggacggcgtctacccggagaaggtcaaccccggcagggagggcgtcggccagaacttccgcagcatcggcaagaacgtcaaccccatcgacgtcaagttcaccggcaaggccaccttcgactaatcacacttcacgtacgccgcacgccccagccgggatccaaattaaagaacttcttcttctattaattacagtatattgtaatttagctgctggta

SEQ ID NO: 55 - DNA, 255 bp, Zea mays
gctcgtattcgcctcatctcgcagcagcagcagaagaacaagggtatggcgaaggatgggggtcccgactggaacggcctgctcaagtggagcctcgcccacggcgacagcaccaacccgccgcgtgctctcaggtatgcaattcccgcgttcagatgccttgcttcatccccttgctctcgtcctgtgccacaaccctaatgactccgtgttcatcagtgcacatgcatagattttttttgtcttactccctgt

SEQ ID NO: 56 - DNA, 681 bp, Zea mays
tgttgcggcacgttgctgaactcgctcccctggactggatgagcagcaactgtagcggctggacctggatccggctcccatcgagccatgaatgtccagacctgggcctttgtgtgctccaacctcacacggcggccgctgaaacctgcgtgactgcctgcacaggacagcgtgcagatgctccggtccggagtcagtgcaggagcacgcgggacttggagcgttgccgggcgatccgcgacttgcaagaccctacacctcccgtactaatactagcatggtgtggcactggcaggtaagaacagccatgaatgggaggcacacacaagtgaggggcttgtgtggtgtggtgctggaactccacgcaggctgcctcagctatctgtctctctttctgtgactgcctatgctgtgcgtgttaaattactacctcacctcctctgccgttggagagtggagagagagtggtgctgagctagcgtgtggggttacgcttacgacggcctgccttgaaggctgccgcaggctaggcttcctgtgtggaagggggggagttttgaccgcccacgtttgactgccccgcgtgggcccgccgcttatgatttgcctctgccccgcgtgggcccgccgctcgcaaactcacgcgctaccgggggcagccggcagccccagaggcccgca

SEQ ID NO: 57 - DNA, 545 bp, Zea mays
caatgaagttgttgaaaattcagtatgtctatttccgcaaaattttattgtctatgaaaataacctggattgcgttgtatgcctaccaaatcatttcatgatatgtttttattctcaaatgtctgcaggaagaggagagtgcgcaacaagttggacgtaaaagagcagctcctaggggtagaggtagaggtagaggcggaggttccactgcaaagagggggcgaaaaacagatattgcttccatgcaaaatatgatgagcaaagatgatgatgattcagaagatgaaccgccaaagaaaactcctcgggtcagaatttcacctgagtgttctcagttcacatattgtagccaaattttgtgatgcgctacacctgaaagcaaacatattgtgtaaaatgagttgactcagtaagattttcttttttggtctttcgcaggtcaccaggaactatggcgctgtcaggaggagatgaccctttaaggagttcttgctcatgagagttataggctaggtgttttgtcttgtaaagttggaagagccgac

SEQ ID NO: 58 - DNA, 390 bp, Zea mays
  misc_feature (227)..(227): n is a, c, g, or t
atctcttttgcttggaacatttgtagaggagaagggataagatcaacgagaagctgaaagcattacaggagcttcttccaaactgcaacaaggtaacatgcattgcgtcttaattttaatccgaccataccccttctgctgttctctttttcgtttgatctcagaatagcttgtttcagaccgacaaagtttcgatgctagacgaagcaatcgactacctaaaatcncttcagctacagcttcaggtactgaatgaaatcatatcagatacacaattgttggttacactagacatttatttatttttatctttctctcctgcatcctttcctgttttatttattcagcctccagatattatgggtgctatagtagatcatcacattgtca

SEQ ID NO: 59 - DNA, 516 bp, Zea mays
tgcctttctaatgtcaaagttctgctccttgcgctctccctaaacaagcacgaagtatcaagcacaaaagtctatatctagaacaaagcatatgatttaaaaccatcagatgccagccagatataagttacctggacatagatatcgaaaaccctcctccctcagcttctccaagactgcatgtctggaaagtcaacctctgcaaattgaagtgtgaccgtgtaatttccattctcaagtccaatgccatagtatctcagggatgatggtgacatccttgctgtttggaacagtgcagagtctagggtgttctcgaactggcgtgaactgtagatgatgtaacttgcattgggtgcatctgcgtccaagaataacccaacgctgctaacggcccatgtaggtgcacccgcaacatagtatgatgcagcactaaggttggcattatcagcttgatagacagaattatctgaacctgatattgctctactaccaccacagtccaccgcaaaggacgca

SEQ ID NO: 60 - DNA, 510 bp, Zea mays
attccttaagggaaaggaagggaagaaaaaaacatctatgacatgtttaaaaagttgagccaccatttgcttggcgacagagcaagaacagagaccggcgcgctattgccatctcgcagccacgaacttagctgtttattcacctgtacatcaaatatggtccgtcgtcgcccggcgtttatcgttatcatcgtcgtcgtcgtcgcccttttggctagttcttcttggactgcagggctgccaccctctcctccgtggccttgagggcttccccgtagatgcgcaccaactgaaaaaagacaaggccattgatgttttgttacccgggttgctaccgaggcgaggcgaggcgaggcttgacgacagtggcgagttggcgggcactgtgaaccgctctgatggcaaacctcgtcgacttcgccgggcgtcatgatcagcgtcggcgacatcatgatggcgtcgccagcgaccctcaccagcatgccgcgcttctggcactcttcgccgaaa

SEQ ID NO: 61 - DNA, 540 bp, Zea mays
gtctgacattatgctcgttcacatgttgcgtctagtgttctctttttgtgcgaatatcaattctgctaatccccctctactgtaatgcattcaggctggaattgctcttggagagttaacttcgctgagtgacctttacgacacccagtcgatagcactgctattcctgatcggtgttgtttcagtcacaccagcattgctgggcaaggatgaggcacaagagaaaccaccagaaatggcggtaggcgcgtcatgatcaccaaagcacctagcggtgtccacgtcacctgtatatatgtcaacatccagcatgcagtggcttcattagttcggatcaacgggcccttgtggctggatgataaaaacggttcgcatggccacacagcttggcgggacctcggcctaaactccctccactgctggcctagagaggcgcctgtgttgccatcatcagaacatgattcggtagccattcaccacattattaatgaatgaacctgggaagcagaattgcctgtagacgagctgctctgatgtgta

SEQ ID NO: 62 - DNA, 869 bp, Zea mays
cttgcatgcctgcagtgtaagcttgaatctgtctttacaatcgtgaaccataagctgagtcctgttacagggcaactctcctttggcagagggttgaagagagcagagagctatggcatctcatgtttccctgccaagaaagagaagccctgaaactggacagcttaggcattccatttcctgatcatgtaaacaatgagttctttcctctcttatcttgacctcacagttttctgctcactgtggggctgcggcactgcaatatttatatagtgaacatatccttgataagatgtgtacttcttgatttttttcgaagggttttaaaccatggatatcctcttggcatccaacatctccctttcccttttgccctcatggttttgtctctctgaagttctcaccccactatcggattccctttctcgttcgagatcgagacccttctttggcaattggcactagccacagaactgtagctgctggtgattttcactttactaagacaaaccgattgttcagtaatttgactacacagtaaagttagtgatgctgccacacttactgctggcgatttccattttcttaagagaaaacaattgtctagtacagcatttgtctatttgaccacacagtaaaattagctgatgctgcctcactaatgtttctgttctggttaagaatacatgatctgaagcttagatgcagatttcactcataatagtccacatagtaacaaacaacatgatacaactccgacaatatgtgcaacctatcaatatcagaatcacaccaacactattgaatccagcattacgtagttcaatagagattcttaatacgcctcgcctactgtcctggattatagg

SEQ ID NO: 63 - DNA, 810 bp, Zea mays
gcacctcttctagtgttaagagcatgtataactggaacaactggcttcagttattcagttatatactactgcctgcactaagaatcctatgagggtgcatgcatttttgctatcaaacaaataatatttagtccacaataataataggaatatccgtactgttttataggtcatcaacgagactgtccgattagccaatattgcgcctggcatcttcagaaaggcactgaaagacatacagttcagaggtgaagatgtccttacacctgcagttttctctgttgagcaaactgatcgcactctaaaacaaacatgataaaaatcatagctttgatgtcactataaagtgcttttgacgtgtgatcttgaacagggttcacaattccagctggatggggagtgatggtctgtcctccagcagtgcacttgaacccagtcatttacccagatcctcttatctttaaccctacaaggttcaaggtttgtggattcagtccatgtatgcagaagtacacatgcagtgatatctggtgaaattgtttttgcacatgtccacatacaggataaaccggagataaaccgtgggtcaagacacttcatggcattcggaggaggcctccgatcctgtgttggagcagacttcagcaagttgcaaatgtctattttcctccatttccttgtcactagatacaggtagaattttcacaagcttatttctccgggagtcagaactcagtttaaaaaaatccatattcattttccaatatcatcgccctttaatttctttcacatcatttcaggtggagaagt

SEQ ID NO: 64 - DNA, 872 bp, Zea mays
cccctgatcctctatagtcacctgcagggttactcgtatagaactttgcaacactgatgcctgaatcaccataaggaagcttcacatatgaacagtttggaagtgttggaggggatctagactgagagtgaaggagatagcgctcaatttctgaatcatcatataaacagatgtgaccagatttcaacaagagtgcaagggcactttgtctgtgcttatttgggtcactaagccctgtaacaagttccattccttggcaagattcagcgataggcaaaaccatctttacaattcgcgactcactctcttcattcagaatcagaacctattcataaacaccaagtggaaggttgaacaacgatgcagttcttactgaacataaacaatcaagatacatcatccatataagtcaacaaactaggaagaaaatacaggctccaacaacgtacagaaaggggcaactttcaaatatttctgggaaatagaaacactttttggccagtctaccttgtcattggtgatatttgaatttgcagatcagccaaccataaaaccattaattccgaattgatattatgaaccagaatcatacagatgataaggcacattgctattagaacagaactccgcatgataagaaaaaggtacaaggacggatataacatggcatttgaaatttatggaagtggacagtctacaaactattatttgagcaacacttaaatagttattatggttaaggggagcgcctccccctgaaagtaggggcccctgaacattgaaacccattggttttacaaacccggcctgcctttttcctgtggttggggggcccttcgctaaaacaggctatttgggcaaccccggcctag

SEQ ID NO: 65 - DNA, 782 bp, Zea mays
gctgctcgcttgtaatatgaggaggtatctgttccagttgttgaggaccttgtgctgatatctgaccgtgcctacacaaaagggcaaattttagaaatggtaatcccactaaatgcaatttgcactcatactgaatgcatgcctggttctttaatactgcattatctaaacgtacaatgatcatagattaggatccatgtatgctacatcagcatacagcagatatggatcttttgctgaaaaattgcaattatgtcctttatacataagtggaacgtgtcaattgtaatctttcggatgatttctccctgttttgctgtcttcttgcatgctatctttttttatacccttgcatgctgtcttctccttgttctgcaattgtatccttgcatggtgtctttttactttacagtgattttttggctaagcgttaaatcttctgattgagcaggaaaagttgattctgaacacgctgcagttcaacatgtctgttccaacaccttatgtcttcatgaagaggtttctgaaagctgcagatgcagataaacaggtgaaatatcactacactagctcaagctcaagatgcagatgatccgtgttttgtctacttggataaacaaaacacctgtagcagtgatcttttggttttatgattgacactgccccactgttgtatctcgcagcttgagctagcgtcatttttcatgctggagctctgcttggtagaataccaaatgctgaattatcggccttcgcatctggctgctgctgcggtttatact

SEQ ID NO: 66 - DNA, 781 bp, Zea mays
ggcacatcaacccggcggtgaccttcgggctgttcctggcgaggaagttgtccctcaccagggcggtgttttacatcatcatgcagtgcctgggcgccatctgcggcgcgggcgtcgtcaaggggttccagcaggggctgtacatgggcaacggcggcggcgccaacgtcgtcgcgcccggctacaccaagggcgacggcctcggcgccgagatcgtcggcaccttcatcctcgtctacaccgtcttctccgccaccgacgccaagaggaacgccagggactcccatgtgccggtgagtacagtatcagcctagcttgccttcctgcttattcgtctcgtccgtccgtgcaagctcgacttatcgatggtttctttgtggacgaatgaacagatcctcgcccctcttccaatcgggtttgccgtgttcctcgtccacctggccaccatccctatcaccggcaccggcatcaaccccgcgcggagccttggcgccgccgtaatttacaaccagcaccatgcctgggctgaccacgtgagtggaaaacaactttcttctaccttcatgttgctaactagtttcactgttaaaagtagtatgaagagtctaaaattaaatagtgaaataaagcctaaatagttgatataaaacaagaaagtaattacaaaatataaaaagtttttctatctacttggatacagatagcaaaaacattagttatatgtaggaaaaaatcatgtatctaaaaagacaaaaaacgtcttatctgaaggtcaaagcac

SEQ ID NO: 67 - DNA, 357 bp, Zea mays
agcatttgtcaaaaccgcgacatgataagacccacaagcaacctccacaacaccgccacccccaagctgctcttcgaccaggcgaggataccttccatcattatttggattgccaagctgtccatacacagggcttccaacagtaaaaatgtgccctgaggtagtcaacccaatcgtaagagtatggccacatgcagccttatggaagttgtaatctatcaacgaagggacgcatgttggtttcagttttgaagacctatcaccatgacccaggcgatatttgtctccatcaccccaagtgaacaactttccagatgaagcatttgactgtgaaatgataacctcaacaacagcagc

SEQ ID NO: 68 - DNA, 796 bp, Zea mays
tggcgcgctggaggaggtcgtccgccgctgcgggtgggctggacggcaagaaggcggcggcggcggcggctgagcggtccaaggccagagggagaggctggatccggagcctcactcggaggcgcgcgcacggggaccgggcgtggtaggtcgccgtcgtcggtaaggaggcccggtcggtcgggggcggggcagcccatgcatgcatgcatggtgtccgatggatggatggatctgcgagcttccccccttgctttggatggagacgtagtaattgcttgcttgtgctttgtaccaagtgagatgagtgagtgcctccttttacctactataggctactatacgtacagttaatagcagctagctgttggtgtggattgctatgcgttagtacttgttaaaagaaagaaagagatatgtatatgtatatggtgcatgcaagaccggcaaattaaataaagtaatgcaatgcaaatacagaatggaaaactaatagaaatacataaaacaaaaaaaggactgcctttttagcagagattgggacagaggtgccaccagaaagatgcatgcatatgcactggggcaacattcgtctttcacaagtgagcaggcaaggtaggcatggttccacatgagctgatccacacccaacaaacaattaattagcgggggaagggtcattgacccatatcattaccaaattaaacccacatatagttgcgcgcaatgcaattgcaacagcagagcagaagaaagtgatacgaccaaactcccagaattggatcaaccaaccaac

SEQ ID NO: 69 - DNA, 255 bp, Zea mays
tttcttcagagtggatgcatctaccaactacaaagagcatgaggttaaagagaaaaagtagctataaaaaggacagtcaagtgacattagctagaaatacagtcatttcaattaaaacttgcaccagagtgcacttccataattacaaccatgagcaattaagcaaatgatctcagaatatccacccccaaattatggtttcatgcttgcatgaccttttacttgtctctgtgcaactaagaagtgactacatgt

SEQ ID NO: 70 - DNA, 356 bp, Zea mays
ctctttggtccattggagtttctggtaatcttttctcatggtttttccaaccaactacagcttacaaagataacttgcatgttttaatgttgttgctaatgtgaacctatcaatagtagtgtttaaatagttttatcaactagagaagtatcctaagactacgctaaagattttttttgatgtaagaactatgctaaaggttgccttcccaatcccgcattacttttatttcagaaattgacatcaagcgcattcatttctatgcccaaatcatcagctaatcctcgcaggcacagaccaccctcagcttttatagaacgccccaattgatatgaatagatttctggatttaggag

SEQ ID NO: 71 - DNA, 340 bp, Zea mays
agacagatgttggaacttggatggtgccatgcgtacatatgttggagctaatgcggtaatggaccgtcggccaaattcaatcgcctctaggtaccacaccggcgacggcgtcttcggcaaggcttgtagcagtagctcatgcaggtagtgatgacaagggactgttagcgaagaatgtcgacagtggccaatggtcatgtaaattgttcatcgtatagagaggtgcaagtgacaaatagccacggttttgtaattctgagctaggattagctattgagaccctgtaattcgtctgggcagacctcaaactagaggtcaacaacgtagcctggcctattgt

SEQ ID NO: 72 - DNA, 762 bp, Zea mays
gttcgctagtatgagaaaattgtacatcgcaccaaagtgttttacaccaaaaagctcggagacagcagctggcacaatagcccagtgagctccatatccaagtccaaccagcaagctcgcaatgtacatagttccaggccaagccatagcaaagaggaaatgcccagcagccatcacgatctgagcaactgccaatgctatgtgccttgggtatgtacgttccctgattgaaaagaatagccagtcaggatgagaacacatataaaaataagtttcataacttgagtgtgcataggaagtaactacaacttgagtgtgcataggaagtaagtctaggaaaatacataatagatacgctcaaaatataataagaacatttctaattatctaaaagctctactattctggagcattttagagagcaaactccagaagttaaatacaagtgtatcctagatgagtatctgaaagggaattaaaagtgtccacctaaactaagaagatgaaacgaagagcatttaatttggtatggaaagttctgattgtcaaagcaacaataccaacaccaatcgtttctaccaagaaacaactgtatcagcctttttcctgagactaaaagcgaatggtagattgcttagtgttggtacctgacaatgatctcagagaagtagccacctccaacacgaccaagaaagttccatatgctcaccagtgacacaaagatatgtgcatttttataaccaatagcctggctcatctgac

SEQ ID NO: 73 - DNA, 560 bp, Zea mays
caaagtgttttacaccaaaaagctcggagacagcagctgggacaatagcccagtgagctccatatccaagtccaaccagcaagctcgcaatgtacatagttccaggccaagccatagcaaagaggaaatgcccagcagccatcacgatctgagcaactgccaatgctatgtgccttgggtatgtacgttccctgattaaaaagaatagccagtcaggatgagaacacatataaaaataagtttcataacttgagtgtgcataggaagtaactacaacttgagtgtgcataggaagtaagtctaggaaaatacataatagatacgctcaaaatataataagaacatttctaattatctaaaagctctactattctggagcattttagagagcaaactccagaagttaaatacaagtgtatcctagatgagtatctgaatgggagttaaaagtgtccacctaaactaagaagatgaaacgaagagcatttaatttggtatggaaagttctgattgtcaaagcaacaataccaacaccaatcgtttctaccaagaaacaactg

SEQ ID NO: 74 - DNA, 255 bp, Zea mays
  misc_feature (1)..(36): n is a, c, g, or t
nnnnnnnnnnnnnnnnnnnnnnnnnnnnnnnnnnnngcgatctagcggacgcgtggggctttctctctacttctgtctgagcccttctgtcaactttcactggttaaaaattattcaatgggagaaagttgctcatctgctcagcaatctggcacaagccaaagtgctgagttgatgccagattcagacaagacagtgcatctaagagaggaactttcagacttaatcgagttcccaacatctgaacttctcaaa

SEQ ID NO: 75 - DNA, 703 bp, Zea mays
catggttgctcatctttgtttgtcgaacctctgaaatggatgactccaggtaggcttgatgttattaccttcctcttggagtatgaatctgtatggctgatgaaatttttatttgtgttgtgcaaagaaaaaaaaagccatataagtcctgcgcatatggttggttcctattgaacccatatacagagtatgatgcttaggtcaccttattactgaaacctgaataagtgaatatacttgatgtttgagcaattgcacaccctgtttggtcttagtagcacggttttattgccccatccttcgttaactaaaacttacatggtttcacctgcgcctttgttcacctagcagtagaagacggcgctttggagggaaagctatcatgcatccactgtggagctcggttgggatacttcaactggtcaggaatacagtgcaactgtggtagctgggttactcctgccttccaaattgttaagagcaaagttgatatcagcaccatctagacctgtgagcaaacgactggcaagtttgagggacgaggagcgcgacagcagtgacgcctgacggtttaatgttttgtgtaccatatgaacgctacgtatacaggtgaagcaatagcgtattttatgtttttccattgccgatcagaagctgtgtgtgacaaataccgtataaaatttcgcatttgctgatgtaacaa

SEQ ID NO: 76 - DNA, 400 bp, Zea mays
aatctgttgcttccgagtattggaaacctcaacccaattcattgctgaaatcactaccatcgttggcaaatataagtaatccaagcaatgtttacagtcccaacaatattctacaaggcaggattggtcctgtgttgagggttgccagtgatagcggtgccgctggtcgtgatggaagaatttctacaagcaacccaattgcaacaagtagtatcatgcatggctctcccgagtcagatgattcttcccaacactcagattcaggcatattactgagggggaatgcaagtaatggtggtttcagctacactagatcgaggcctgttgataatgtcatttattctcaatttatatcaactatgtattctgtttctaaagatcctgacccaacaattgcaac

SEQ ID NO: 77 - DNA, 321 bp, Zea mays
ggagtgatgtattggctaacagaagataaaggaacaagctgtggcatggcagttgtatcgtttgatctcagggaagaaatgtttgccttgattcatcttccagcattggaggtgaaagagacagcctcttgtgccaccatcaaagttgcttactatatgactgagatagatgacaaagtgtgcgtagtgactatgccataccaaagccatgcaccgaggtggcgccgaaacaacgcagagctttcaggcaggactgacatttgggctcttgaaagtgacaagtggttcctgaaatatagcatccagtcaccatctctatct

SEQ ID NO: 78 - DNA, 396 bp, Zea mays
agacggcccaccgggcggggctgaagctgccggcgtacaccacggtgcgatcgggcccgggccactcgccggtgttctcctccaccgtcgagctcgccggcttgagcttcgcgggcgaccccgccaggaccaagaagcaggcggagaagaacgccgccatggccgcctggtcctcgctcaaacagagtacgtcatcggcccaatcccgaaaacacaaaccttgcgcatatctttacgcgtcacggggcatcattaatggcccccgccatggtctttgcagtgccggaggctcgcaaggagcccggcgccggcgacgagcaggagcacgtggctgtcgccagagcgctcgcggcgctgaagccgcgagacggcggcgacggaaaggcggcgccgcct

SEQ ID NO: 79 - DNA, 255 bp, Zea mays
  misc_feature (1)..(28): n is a, c, g, or t
nnnnnnnnnnnnnnnnnnnnnnnnnnnnggcaaacgatacagcccttccctcttcaatcctctcgtcatttcctctcgcttctgctcagctgcaacccagcgcgtagatcttattcttggacgagaacgatgaggagctcttctccatgttccttgacgtcgacaagctgaactcatcgtgcggggcgtcgtcggaggccgaggctgannnnnnnnnnnnngcnnnnnnnnnnnnnnagggggctgagctggccg

SEQ ID NO: 80 - DNA, 804 bp, Zea mays
cttgtgatgtacgatttgcctgccagaaaattttactaatgtaacctgcttgtgatgtgtcagcagctaagggtaataacttgattccaacaagagaagacgatttatcgccaaagaaaagaacatgtttgaatgacaataattctgagagaacttccagagatatggagctagtccacattatggacaatcagcagaagcatgacacacagagggatgtggatactatgagaacaacatccaggggaaatgatgagaagaactccatcccagcacaccagttggaactttctcttagaagaactgactatggaaaattagagaaccacgagaaaaatgatagaagaacactgaaccattcaacttcatctgcattttacttgtaagtgctcctttctttggcttttcataatagcgatactttcgtcatcctaaaaacttatgtgaagtagataagtctagttacagatctggttgttatttttatgtcaggtttttttttgttcctagtgtaggtacagggtaacagaatttttttcaaataatttttgacatttatcttttcttttccaagcatagttaagagacttcttttaatagtgaattaccatgagatgaatagataatctcgaagcataaaatatctaactattgtagacacaccataaactttgagatggaatttcaagtgcccattgctgtatcttactggacaatttgcttcttttcgaagttccacaagtgcccattactgtatcttactgtggaagtatcttgtcgacaggtataattgcaggactgcgt

SEQ ID NO: 81 - DNA, 455 bp, Zea mays
cgccgtcatccctcgcgtcctcccacctctccccaatcgcggcggtgtccactcccgccgtcccacatcagcttcgcatcggctgctcccgccgccgcgccgggcgcatcgttgccatggctggatccggcaaggtgcgccgccgccgccgttctttaaccagccgtctttaatctgcccctgtgatttctgcgctcatgctcccgcttgactctcctttgcagttcttcgtcggaggcaactggaagtgcgtaagtgcttgttctgcctctccagctttacgcgccttggggtctagaactgatgatcctggagatgctatgtaactgtatgatcttttggctgctgccgcattgggcatttcaacgggaacgggacgagattttgtcgtcatccatatatacatcgtggtttactctgtccatggccacttctacaaatagaatcgcttgtct

SEQ ID NO: 82 - DNA, 255 bp, Zea mays
gcatgcctgcagcgaggcgaggtgggagcggaagcaccgcgtccaagaccatgagcctcgtcggaggtgtgagtgcccacgacgagccgaggacttcgatgggagcgagtgagccacttcacggtgagcgacggccactcgagcgggtgggagatgacgaggtcgtaaagcacagccgcattcctcttccagttccggtactccatctccgccgccgtaggtggggtgcccgccggtgtggacgatccgccgtcc

SEQ ID NO: 83 - DNA, 255 bp, Zea mays
  misc_feature (38)..(58): n is a, c, g, or t
cttcaccctgcttttcgtcttcctttcaccccgcttgnnnnnnnnnnnnnnnnnnnnncacctctctcctgcgccannnnnnnnnnnnnnnnnnnnnnnnnnnnngccgccgcacgcctnnnnnnnnnnnnnnnnnnntaggtcccttagctcggccttcgtcggggttcggggtccttcgctggcgacgatcacccaccaggtctcctttgaaagaagaattaggcagcacatctttacgaattgaggatgtca

SEQ ID NO: 84 - DNA, 530 bp, Zea mays
gcctccggtatctggtcagtcacaatatacagttaagccgcaccctatggggttgctgtctgaaaaatcaggtactcatgtcacaataagtgctccaccagcaaaatctgatccacctaagttacatgctgctggggatgctgcgtcaagtctgaaaaaggataatgaagttgtttctgggatcactgttttaaataaaccaacccttgagagggagagtaacgatcctttagtcacagtgaagcattccactgtgataagcccgtccttgccaattcatggtgaaaaaccacaaactttggtgacatcgtcgcctgttgctaattcagtctcacctgtgactggagctgatgggaaaagcaaagaacccattcaaaggtctggatcatttaaagatagtaagaaaaatgtcactaaaaaggatgtcaggaattcatatgaaccaccaaatgtacgttgttgttggcatctttctgccatgctatgtttgcatttttcctactttagtatttacttttgttcatatattt

SEQ ID NO: 85 - DNA, 548 bp, Zea mays
tgagcctgggagtggaaagcttcactgtttccgctgttgaatgtgacgaaagcaagaggactcacaaagttgtgaaagatcctagcaaggataatatttcttctgatgctaccaaacatgaattgcctaaggaatgtgcagtggactctactgaacaagcatcagcagtgacttctaacactgatgattcatatgcagcacctctagtaactaatcaggagcagttgctgaaagggtctgcttctgagaaacaagctgtgatgaataactcatccatgaattccgatacttcagcacatttttttgatggcaatgatgtgggcgtgacaacttctggaacttcagagcctactgctcaaacttccaatgatgaaggtgactcagatattagtccagaaacagacctagctgtttataatgtccctctaggttcatctgagctacaactgaaatctgagagtagggcaaatgatcaacagcctgctgcaccaacagtatcagttcgaccggtatccagggaaaagcctagtacaaagtccacagctggg

SEQ ID NO: 86 - DNA, 434 bp, Zea mays
tagcaaggataatatttcttctgatgctaccaaacatgaattgcctaaggaatgtgcagtggactctactgaacaagcatcagcagtgacttctaacactgatgattcatatgcagcacctctagtaactaatcaggagcagttgctgaaagggtctgcttctgagaaacaagctgtgatgaataactcatccatgaattccgatacttcagcacatttttttgatggcaatgatgtgggcgtgacaacttctggaacttcagagcctactgctcaaacttccaatgatgaaggtgactcagatattagtccagaaacagacctagctgtttataatgtccctctaggttcatctgagctacaactgaaatctgagagtagggcaaatgatcaacagcctgctgcaccaacagtatcagttcgaccggtatcca

SEQ ID NO: 87 - DNA, 398 bp, Zea mays
tgacttcggcacagtggatgacattttcaaagcttgcccttcaactacagtgtccagttatatcattcagaaaacaacaagaaaggctccattcttttcaggcaaataaaaaccatgcaatttcatggcccatccattagtagattctcttctcaattaatagcaactctttattgaccattttataaacaataacactaatgaacaatctgcacaaacaaattgggatgtgctttcaaactcctcttgtaaaaagtcacaaactcacaatagtaaataggagtaggaagcatggggttattaggtcagtgatcagataaacagatctagggggtatttgaatgcactagagctaatagttagtggctaaaattagttgaaacatccaaacaccctag

SEQ ID NO: 88 - DNA, 255 bp, Zea mays
  misc_feature (1)..(28): n is a, c, g, or t
nnnnnnnnnnnnnnnnnnnnnnnnnnnncccaaatgtacacatatagaactttctggtgctctggtgctcttactcttcagaccatagacaatggccaccgaactagatgtgagcaagtctcgccgcttcgacattgccatgtcgaggcgaactcggcgatcaacaagcctggtagcctgcttccaagatcagtatacaccgtccctggcacatcatcgccaccaagagctgaaggcatttctgcaacgccaaga

SEQ ID NO: 89 - DNA, 447 bp, Zea mays
agatgaggtccaatccttaacgaaatatcctatgtacctgagtttgtctcttgatcaaagaatccgtccccgccatcgttttctggtttccttgaagaaagctccaaagggcccatttcctctgagctcctttgtgcccacagatgagcgtttttgccagaggtgggctgggactagcttggaggcataccatacattcagacagaggttgctgctaacaagtttcacagagaagtctggaaggaaaacactagtctcaagacggtagatgcctaggtggtgggagtttgtttctcttgagcccacgctcctcctaacagtaagatgatctctttctgattttagagtttgtttggtagcagtctgcatacgcatagtactgattagtgattagttcacagttctcaggaaatgggcttggttgctgacttggtacttgatgttcac

SEQ ID NO: 90 - DNA, 818 bp, Zea mays
  misc_feature (706)..(708): n is a, c, g, or t
tctttccaagtgtataatcagagaaaatgcacagaagacacagcaatataatgcttcagttgcaaataaatttccaccccatgttcctcaaggtaaacacctgtgattccgaagaggtcattgcggttagctgtataaactgatttgttgttaccgtacctcaatctactaaatagtactccctccgttctaaaatagtattcgtcttagctcttgatttttatgtctatattcaactagatgttgatgaatgtagacacggatatatagaatacatacaccaaatattgtatgaatccattaaaaagtcaaaacgaattttattttgggactgagggagtaatacaaatacttttgttggattcagtgacaagatgcatccatataatagtatttcagcgaaacaaaccattttttggagaatgtagttaactgacgtgctgttgctgacaacattattctaaaaacactgatttgttatatggatggttgcggataacattattgtcaaaatggtttgtttggctgaaattttatttcacggatgcatgttccactgaaataaactgatatattatgcatcctgtatgaactgatgtgttgttgctgataacatgttaagaagtggaacaattttgcaactctgaacaggcagtttacgttagctgttataaactgatgtgttgttagttgttactgtacctcnnnctaattatcttctgtttttcttcattaacgttctgttattttggagaacggatacctggnnnatatactagacttgctttatnnnnnctgattgctgannngtgctgctg

SEQ ID NO: 91 - DNA, 649 bp, Zea mays
gccaatgaaagatcaccccttagcagtaagaacattctgttgcaaactaatgcttatattagccatcagtttatcagccttttactactttgcatgtagttggagtgttcgaaatctgatgctcaagggcagcttggcagaactttggaggatgcacagaacataactgaattatcaaatcaggagaacctgttacccacggtgtctcctggctttatatcaacgtctaatgggaaaggtatgaacttcagaaactctacccatggtcaaaatgggcatagtttgctacataactgaggcatacatgaaagtagaagattgatgattggatttctgtgtgcaggtactaacatacatgatactcaatttgaggaagagccgacaggtatgaacaacagccttattgaacctaatggaaatgatgacatgtgcacagaccatgctgctaccaaattagctaaaagcaaccatgttatggaaacagctaccctacctattgatgggagttcacagtgcatcattgacatttctccattgaggagctgctctgctttggagagatctttgccttcaccaccgcaggcttcgttcgtcaagaggtcacctgtgtggcatgtagttgacgcaaaacatgtgtttaaggagttac

SEQ ID NO: 92 - DNA, 437 bp, Zea mays
gttttctctgttcttccttgttcagaaaaactgaatgaactgcatacaaccaaaatgattcaatcaagaagtatcctgagcgcttatcgcgacgtgcgtttgtgcatcttctccacgctggcacgatgcgtccatgtccgcatacttctgctgttctgcgtccacaagaatcggagctcgcgcgcatgcaggaaatgaacacaatgcaggaacagctaaccgaactaactgaatcgcgtgcacaaagcttaacaaatcgaacacaaaataggtttagcagacgtatggttttagagttcaagagctcaatgtagacatcaacaaaagttcgtctttaattcagcgtgatttacatgacatacaaaatttactgtgctacacgtgttttaatagatgtgtgcatgcttgcaggttatcctggcagacaccattgaagc

SEQ ID NO: 93 - DNA, 624 bp, Zea mays
tgcagctagcggagcagatcgacgcgggcagcagcatgaaatgatcggctgtagcacactagctagtagaaagcaaatcctgaggcagcacaaacgaaccagaaggcggctgctgctgccatgccagccaactgaccaccatgcatggaagtggaactggggaagcaggcgtggaggaccagacagcaggccatggtgggtggtggccgaaaacggcaagagcagcatgagctttcgggtcggggatcggcggctgcgagccaaaaacggcagcacagccaggttggtggcatggcatggcatggcatggtatggcgcacacacacaacagcagcacgaagcagaaagatgaactgatggcgaacggatggaccatactgacaggcagctagcatagcaggcacgtattcaattcaacaaacacactgcctgtgtgacgaattgaaggtggtcgtacgtaccgggagtccgtggcgtactcgtagagcttgcccttgggggagaagacgatgacggcgacctcggcgtcgcagaggacggagatctcgtgcgccttcttgagcaggccgttccggcgcttggagaaggtcacctgccggtttatcttgttctctatccgcttca

SEQ ID NO: 94 - DNA, 434 bp, Zea mays
cccaattgggtgcacggactggagattctatgctccaaaggtgaggggggattgattgatgcgagtttttttgcatgcctttatttttcccttactttgtcttttgaggttgatgcaggcccgaaacaaggcttcttatcttctgcaaggttcaatcttgaaaaaaagcagtggcgatggtaaaagttgtgtccggtaatcaaaggggaccaaatgtctggtgcagcagtgcccaggttgtggttccattcgatgggcaacggcgaagtgctcatatcattccatgttttatgtacaattcttttttagttgttgtgttttagttcaaaaatgaactagcgagcgacaaatattcgagaacagagttagcagccagatttagaggggtgttgattgatgtatctgtgggggtgcttctgcttggttgattgaca

SEQ ID NO: 95 - DNA, 255 bp, Zea mays
gatcctttttagtcgacctgcagcgggagattattccacttggcagcgcccggttcggtcgtccgtgcgtgccttgtggatcgtgctctgctttgtacttggcacactcttctttccttttcagtcgaaacggggaacgcctcttgggcttggctcttgcttctcccccgacgcgagcctctttgatttggcgtctctttttccttctcccccgacgaaacgggaaccctgaagtgagccggtcgggccacggga

SEQ ID NO: 96 - DNA, 811 bp, Zea mays
ttctgtatgatttcaatagtaaaaggcggcaaaggaatgcaagagaccggcggacagtagaatgacatcgtacaacacattggtccgaagactgtgctagcactgtcagcaacagtgtttgctcctttaccaagaatcttgtgcctgtccaaacgatagtaggcttcagagcttttgtctggtagtaagtacggggctatggctgttgtgtactatatatatatgacatgatgtcctgacaaatatatatgtatcgataacttcagtcaatataatgtcgaaatgggcgcggttaatgttctggttcatgtgtataagtgtattgacagcaccaattaactaacgagcttatatttcctttcatcatcaggtaaaaaaaaacgcaaaggaatggaacatccgtacaaaggcgaatgggtttaactaattaaagtgtacatatatcttctctaaatttttaaaaaataactcagcaaaaggaaaaaaatgacatgctctatatgtataaatatcttgtttttatattgtttactactacgtatatctattttctgaattcattaatatttcaaccatgcatgtgagggacaactaagttttagtttgcattcttcacatataaattatacatatataaaaaaaaattctcccaaccttgactttattgttgattagatgcaaagtgttcctgttttcttcaacagaatcagaccagggatcgaaacttttttgtgatattaacacaacattcaaatatttcataagcataggaagcatgaccctttttattcatgcatcgagagaaaaagaa

SEQ ID NO: 97 - DNA, 255 bp, Zea mays
tctacagaaattgcttctcgtggtaggatgtcaagccactctgctgaatcgaagtcctgcttggagtctttgcgtggcgaacttaattcatcaagtgggttcatattgtctctcctgttccatgctgcccactctgcacggaatttgaatcgtctcgaggttcttctgaggtttgaaggtatccaactattagcaggctctatttgctcttgtatttcgggttataaagacattctgaatgccactaagcgaaaa

SEQ ID NO: 98 - DNA, 597 bp, Zea mays
tgccaggtaacggccacggattccatcactggaccgcctgtgtcctcgccaacagcaacccgcacgccggaaggggggttgcggacgccttcagattatcttcaaccattcctcctcctaaatcctacaatttatactttcagtttatatttaaataacacttccataactatatattattcatatgaacccaacaacaacgccgacgcctatataaacgtagttcacccacccaaagttgcgtgcagcagagcagcgtcgtatcgtattccatcctttgcttctattccacttcaaatcacaggcggccgcgcatccatccactccccccctctttcctgtcgcatcgcattgcatgctttgctccccacgcaatcaatcggagatcgatctcgtttccctctcttccttctcctatacaataccctgctcacttcttgaattgctccgcgccgaaacgcgcgcatgcaccccatgaacatgacgagctgcgaattcgccgccgtccgcctccctggccacccggcgctccggccgagccgcagtcgaccatctccgacgcccccgccgccacccgccgtagccaataagaaacca

SEQ ID NO: 99 - DNA, 255 bp, Zea mays
  misc_feature (174)..(198): n is a, c, g, or t
gcgcgcagtcttcccacgcgtccgggcgtcgtcagaatttcctcttttttttcctccccaacttctccctgcgtcggccgatgctgcggcgactgaccacagctttcgaccctctccagtggagggaacggggttagcgagcggcctccgctgcgacggcaacggcgtgggagnnnnnnnnnnnnnnnnnnnnnnnnnctccccatgttgctggctgcctcactgctctccgtcgccgctcctgcgctggctcga

SEQ ID NO: 100 - DNA, 356 bp, Zea mays
catatataataactactgtaggcagcggcatctcctgctgggacgaaggttcaagaagctagctaagagagaagggcataacgagataataagcagcgcgcgaagatgcaagactgggcgccggtgttcgtctcgctggtgctcttcatcctgctgtcgccgggcctgctgttccagatgccgggcaagtgccggatcatcgagttcggcaacttccagaccagcgccatctccatcctcgtccacgccatcctcttcttcgccctcgccgccatcttcctcgtcgccgtcggggtgcacatgtacctcggctcctaggcggcggcgcggggcgccgtaccttctctccctctcta

SEQ ID NO: 101 - DNA, 692 bp, Zea mays
tataaatccgtgcatttcagtttttgtttcttttttttcttctcctcatcatcatgaagtttttgtgcaattaggaatgctaatatccactcttcactagtatgacgagttctacattgacctgtggtgatcatttgacctgaatacttactttgtcctgagcttcccatctcctttacattatttggctggctgttccagtgtctgaaattcgcgttgaatatgccttgctaaacgcacgggcagttctgtactcaccaaagtatacggaaccctcgattcttacttcttaccgtcgtctgaaaattgcaaccggaatcaacacaggcagcccgaagacaggcctgccgggtgcaggaggtggccaggaaccgctgccagcagatgtttgcggatctaaactcccactgcagaatccccgtgagtagttgctatgccctgaggcctctgaatgacctagctggttcatggacgctaacctgtcgtttgaactcagggaccaagggttaggttcgccgaatacgtcgaggagaaggccgcgtcgtctccacgcaggagcagccggaggtgactggatttcaggaggtggcaggtgaactggcctgcccgatcgtgttgctgaattgttcggtacaataactaagtagtgctgcttggcgaaccatcacccttgtcactggtttgtgttggtt

SEQ ID NO: 102 - DNA, 517 bp, Zea mays
tgcgagccaccgatttttcttttcatgaaaggaacaggtgtgttttcagatgcaaacatggccgtgctcccccatcagcaaatgcatataatctgtagaatgcaaatcagtctctattcctgtatttttatttagtgacacggtcatgcttaagtagactgtacaattttttttttcacttgcagcatggtgtggtgctggtgctgaccattacgagcctttgatcgccactgtcctccagcatgttactccagacaaggcagctgtcatcctttgagaatgactgcagttttttctacctcccttagcttttagtcagcggtggagttgagacaattttgttattggagctagagtgctgaattgaggagcgacatcaatatgcccaagagattagagagacggccaacctgagtatcttgctctgttgcggggttttgcctgcttatgaagctgtgtatggtgattttgttgttgtatgtttatgagtaggctctgactagcgaggatattctga

SEQ ID NO: 103 - DNA, 255 bp, Zea mays
tttataccaaatatgccaagttgcaacaaacagcctaggctggaactcaaaactgtactatccacgcggttctcctcttacatttctcaacatcacgaaaattaggaaccaataacaaacccattatacaatctctgccattgttcccacagcagcaagcggcagcaatcagtatcggctataatcctgtatacatgccccctaccaaaaatctttttttatgtactcgctttgctctacaaattaagcaaacta

SEQ ID NO: 104 - DNA, 429 bp, Zea mays
cagctgtgcgacgtgggcacgcgcttcatcagctgggacggcgtgcacttcaccgaggccgccaacgccatcgtcgccgccaaggtgctcaccggcgactactccacgcccagggtcaccatcgccaaactcgtcaactccacgctgcccaacgacgactagctggctggccgatcgatcgatcgatcgtcgtcgtcgtcgtcattctgataaacatactgcaggctgcaggcttcatcgatccatatccaattgtccgtagaaatctacgactagctcattgtagatccgtaatgtgggctaagagatgcatcatgcatggcgatcgactatagctgcccaactcgtcaagagctggttcaccgccctcgtcatgatcgagctagtttggacgagcaaattccatggcaccgagtaacacaattgata

SEQ ID NO: 105 - DNA, 255 bp, Zea mays
  misc_feature (33)..(38): n is a, c, g, or t
tgatgacctatatgatcatggtcactcagagcnnnnnncggncgcgtgggttcgcattcagtggtgtcctattgggcgcctagaacttccatccttatttgaagaaggagagtccnnnnnnnnnnnnnnnnnnnatgatttcacgggacaattcaactctgagcccatggtaccggtacctgccacacctcatcctggtccgtatctagcttattttcagcctgccccgacaccggtatcatcaagctcccttgt

SEQ ID NO: 106 - DNA, 459 bp, Zea mays
atctcctagtctcaaggaggttacctgggggtgcactggtggaagtccccaacgctcgggacatcaccaaggattctcctcgttcttcccacccctacttcttgagttcttgcccacaaccctttgttgtgttgcttgctgcgcatcaatcccctgctagaccacgaaggtatcatcatttacttctttaatgtagtagatgctaatttacttttccacaaatttttgtcaaacttaggacaaactcagaaattgttatattttaggactaagggagcagtatttatctatgccatatgatgaaatggtggggtggaggactggggcacgaacaccttttataacaatggctcgtggttcagagttggttgaatcctattgatgtgtgaaacacttgatccccggtgtcgggtctagcggaaatgtgtaatcaaagctaagcacataagccactgtaga

SEQ ID NO: 107 - DNA, 596 bp, Zea mays
gaccttaaagctagtaacattctcttggaccaggatatgaaccctaaaatttcagattttgggttggcaagaatctgtagctccaatgatgctgaaggaaacacaaagagggtggtcggaacatagtaagaacacatgagtttatcaacattgttagcttgtttcctcttaattagtattgggtgatcaagcaattctaatatgttaggataacatttttatcttgcttaatataatgatttcagtggctacatgtctccggagtatgcctctgaaggcatctactcggtcaaatctgatgtgttcagctttggtgttctacttcttgagatccttagcgcgaaaaggaattcaggttttcaccaatatggagactttcttaaccttctcggatatgtaagtatatgctttctccacaaaatgcacgcaagttgttatggcataacagctgtacagatcatggaggcttgatgagctttctgctgttctgcagacatggcacctgtgggagataggaagatggcttgatcttgtagaagcatccatttcgaaagagatgcatgaagcagaggctaggcggtacattaacatcgcac

SEQ ID NO: 108 - DNA, 255 bp, Zea mays
tgcatgcctgcagagagatgctgctaggctgctagctggtacggcagctacagtgtcaaggcgctactgcttgatctggtttataattttctcaccttttgttgttaacaggaagtcgtccaatgagcctataactccagcagtaagctaaccgtgcaatacttcattgctgaggaatatttagcttccttattaatagataattaatttggtacggcaatctttgcagaattttctgtcgttgaggcattcaac

SEQ ID NO: 109 - DNA, 255 bp, Zea mays
  misc_feature (1)..(30): n is a, c, g, or t
nnnnnnnnnnnnnnnnnnnnnnnnnnnnnnccaaccttcacatttccacgtatgtagaatactccacctccacccccgtccatttcccatccccaacctctctctctcccccctaggagtgggagctcctggctccatctatccgcctgcagatatcacaagcttttcnnnnnnnnnnnnnnnnnnnnnnnnnnnnnncttctccgtctgcagtgcgagcagctgctaaaaattcccccataagcctctggcttc

SEQ ID NO: 110 - DNA, 343 bp, Zea mays
gagagcgctttcctgataagcactttgatcttgaaactgtccttcatgcaaagatccgtccactcacagttgctcacagtaagtcattcattgggtttttccatcctcaaggcactgaggatgtaactggattcgacttcctcgacggtgcaatgtcattcgataacatctgggacgagataagccgagcagcggagtgctcctccggaaagcctgccctctacattgtcagttggaacgaccacttctttgtcctcaaggttgacgccaatgctatttacatcattgacacgctcggtgagaggctgtctgaaggctgcaaccaggcatacatcttgaagtt

SEQ ID NO: 111 - DNA, 420 bp, Zea mays
cctgcaactgcttgggtattgtacatctgtatgtgatgtgtcatgtgcacatgctttgccattttgcattggcaataataggttgtgcatcccttcagcgctacaaggagcaaggatgtccgcttcagattaaatccgactgtttagactgctgcaactgtaatcatagagataaatacactatagaaacagtagtaggagccgatacgaattaaaaacaacgaacaggctgaaagatgcagcgcttgtctgcacaacatccccaaagtatcttcgagtcgaatacaacagggtaactgagtgtaaccacagttcgtacagaatttgtagagaaaatgaattgtccaaaccaccggataaattcatcttgttacaatgtttgctggcagaatagctcgcacagcacaggagatgagacca

SEQ ID NO: 112 - DNA, 565 bp, Zea mays
gcacgcacaacctagttcatgtttcttgcccatactgatgttcggatgctgtttctggtgttacctctgcgtggaaatctgaatatgctctcatactatgttcaggctaaggtatttttctctttccctctactctggtcaatttccctagttcatctaaataaaattttcaggcagattttaccattcatttgagctggacactaacaaaaaaaattgttatccatgtgcagaggcttgtgatgaaatacgggtcacttgtgtctgttgtctcttttgttggactgtttatctaccttgtcgcgagcccgtcgaagagcagctcaaaggcaagcaagaagagacgctgatctacttataggtcgcagttgacggtgtagctggtggttttgcctggaggaactcttggtagtttgagctgtgcgaaagcttttaagtttacatttactcaccactccatgaacggcaacgaagtattcatcagttagaaaagtgcacctataattttggttgcttataactttctatttgtcagagatttaactcgaaggacctgtaactagga

SEQ ID NO: 113 - DNA, 708 bp, Zea mays
cattgctcgcggtataacatcatttgtcttctttcctattttattgtcctttcccttttgtgctgcaagttttaaccttttccatggtacctgtcatgctagtcatctgggaaagcattgcggaaggataaaccttcacaagatgtagaagaaacctctgttaaagaatcaaaaccatcagctggtactttgacattttcctctcattttcaggcttttatttgtgatatgtggtagctccttgcgtggatagttgattactgcctcttttttttcttcctgttctgtacaatagcaattttgccagaatgttctgatgatcagtcttttggtagttgacttaattttattagaaatagtaataatgttagtttgctgaatattaccctcccatattgaactggaaaatggaagttttagtgtgaagtacatttttttcatgtttgacagtttgagacatttacaccctgtactcttccaggtgaagatgattcgtcaaaaatatgttataaatgcaaaaaggctggacatctttctcgcgattgcccagaaagcacatctgaggtggaccgaaatgatggcagtattagtagaagcagagacgatacgggtaccaatactgctcctgctggtggcaacagtcctatggacgaagacgatgttcaagaaattggtgatgaagagaaagaaaagttgattgacttggac

SEQ ID NO: 114 - DNA, 353 bp, Zea mays
atgatggtagttgagtagttcttcttgcctctcgatcttgggcattgatacttcgcccccttatatggtgcaaggtatccttcgtggttggggtaccctgagtcaacaacatcaaacttacctgcaacatattaatatgcaatgagcgcatacgaaccatatagtaatattttaagaataaacacttgtcatgtgaatttacctttcggtggatgaggaaacttgtcaccatatttgtctaaagcatcaccgaataccctcatgtccatcccacaaccacaaatgtaaatcttatatcaaagtcacatattgccacatattctgacttgagtaaccatgacgacccacatatt

SEQ ID NO: 115 - DNA, 373 bp, Zea mays
tgggcgccgtggtgcgggccgtgccggatgatcgcgccggtgatcgacgagctggccaaggactacgccggcaagatcacgtgctgcaaggtgaacacggacgacagccccaacgtggcgtccacctacggcatccgcagcatccccaccgtgctcatcttcaagggcggcgagaagaaggagagcgtcatcggcgccgtcccaaagtccacgctcaccaccctcatcgacaagtacatcggtagctcatgagcatgccatgccatgccatgcatgcatgcatcgacgactaaacacatatgtgtatctctagtagcctttgcatacgggatatgatcaattcaagcagccagccgtctgctactgctgtgtt

SEQ ID NO: 116 - DNA, 806 bp, Zea mays
gagtgtcgggataagctgggttcttgacaccggtggatgcccacaacaacctctgtttcttggcacctttcttggccaacgcctcccacctcgggccagagaatttcttctggtagagccgatttgctagtttggcctgtgcgacggcagcctggcagagcagaggatgttcatattgcataactctgtaaacagtttgagctctagcattaagaagaagctaattctatttttatttaccttccctctcaaggcaagtgcctcaggtgttccaatcttctcaagcattttgtcgataagggtgtcgactcggctgacaaagaaggatgcgacactggtaactcgagataagtcgctcaagccagaagcctctagcccatcaaggtaagcatcaatcacagcctcgtatctcgcaatagagaaaataagctgcagtgacagaaacgaatttgtgatcaataagcttaccgcagcagttatactatagagttctaccacaaaagaactactccgtctgtcccaaattaaaattcgttttaggtaattagtggattctctgatgtatgtgttttatatatgtgtctagattcatcatcgtctatttgaatataaacactaaaaccaagtgctaaaaagaatactattttaggatagagagagtatcatataacagaccagtggcaaggcagcactctgcaattaccgtgacgttgacgctaatgccattagcgataacttcccggatggaaggaacacattcggcggtagcagggatctttatgtagacattggggcggttgaccact

SEQ ID NO: 117 - DNA, 255 bp, Zea mays
tcatgatcgaggaggcacttgataatggactgacgaacctgaatgagaaactggtaacaaatccacccagtcagtcgatatcaatgcggttgacgctgcaggtattgtcatccgtgtaataaattctgatatcggttgctttgctgatgcagatggagcactgggaaaggcgtgtgacaaacgtacgagcgtaatctggctaatgattctgtacttgtgaattttatttgaacatgtgtatggttcttttcagtt

SEQ ID NO: 118 - DNA, 255 bp, Zea mays
  misc_feature (42)..(59): n is a, c, g, or t
cacctcgcggacggcggacataccccgtctcggttctcaaannnnnnnnnnnnnnnnnncccaacatctccccgtccaggtcgttgggtcagcccctatccttcaccttcacgacttcacctgcttctccactgcgcgccccgcccccggccccgtcgtgcgccctgctcagatccatcgcacggggcttgccgcaccactgcgccaagcaaaaggtgggcggccagcggcgcgtgcgcagatcatgcactggta

SEQ ID NO: 119 - DNA, 397 bp, Zea mays
gtcgccacagattccatcaagaacaccgtacgttccagtgttccaacagtctctctttcgcttcctccttccgccgccgccccaaagcggctctggcggcttcttggctactgatttggttcggcttcgcttgtgcaggtgtatgtcaaggcgaaagagtgcacggaagtggtgtccatggaggaattcgccgtgattcttggaaggcatttcacctcgctgtaccctcaggtatctcgaaactaattgatgtatccgggtttgctgctgagggatcaagatgggatttttatggatctctcttcgtgctgccgtttcggggcaggtttcggaggccacggtgactatcgtggagcgccacatgggagcgtgtgactgtcgacggaaagcctcattc

SEQ ID NO: 120 - DNA, 280 bp, Zea mays
  misc_feature (238)..(243): n is a, c, g, or t
ttttgagcttcccccaaacgattagatgacaatgagagaatcaccaacctctcagcacttgtattatagtcctccacctctacttctgaactaggatcaccaaccaagtaaggaacgaagcacagcagcgagctcaaacgatgaatcgctctgtaattttgtaatccgtcatgccaagaagctggaaaaaaaaggaagcttactccttcacggtctagaagccggctttgcggccccnnnnnncgactcctgcggcaggggaggggnnnngggagtggaa

SEQ ID NO: 121 - DNA, 497 bp, Zea mays
tgtgcttgctttacccattcggtggtgaggcgttggtggcgcccgcggccgcggccacgctcgatagggcgtcggccggtgacgagctcgacgaggaggacgccgaaggagtagacgtcgctgcggtcggtgagctggcttgtgcgcaggtactcagggtccacgtatccggccgttccctttactagcgttgacacgtgggtcgcttcagggttttcgggggctaggcgcgcgaagccaaaatcggcaacctttgctcgcaggtgctctgttaggagaatgttggacgacttaatgtctcggtggatgattggatgatctgcttcccatcgtaggaaaatatttgtcagacataaggatgctgaaggtttggtatccgcagtagatgtcaatacatagacatgttggtggctgtcatacaaaattctgatcatggaactgagatggtgattttcatgatggttgtctcatctagggaactgatactgaaatgtacc

SEQ ID NO: 122 - DNA, 770 bp, Zea mays
atagtcatacaagtggaactatcaatgagccacgtgcaaagaggaagaaactgaaatgagccatacggtttgcctccggactcaaggtaccaatagctcttattttttctagattgattgaatatacttgacacattttgctttcacaataagagtaattgtgcctgaaagtgacttgtatttttactttttttttgtatgtatgcttgcctacatatttagttgattatttacatcaccacattgtcagaatatatatattcccctactccgtccctttaaaattgtaagtcgttttagctttttaggtacataacttctgttgtacacctagaatcctagatattattatgggcatgtttggttttgacccctgacaaaaggtgccttgcccaggcagctcccaggccgtcgatttccagcacctgccctggccctggcggccggatctgcctggtcgagtgccctaggccagtgtcccaactagacatatgccctatgtctataactgattatctacgccacaaaatattgccctcattgtctatagtatatgctgatgtggactccctccattcctattgcaagttattttgggtttttcaggtatagcttttgctatgcataatgcatcaatatatatgccaacacagcaacacctgctttgcaccaagcgctaatacatctgtctcacagattggacagcgcagcgaaccggcttcatccttggtctctctgtccatgcgcgcacatgaagtcacgaaaggtgc

SEQ ID NO: 123 - DNA, 780 bp, Zea mays
  misc_feature (688)..(693): n is a, c, g, or t
tcggagcatcaccgaggcgctggacttctcgcaggtccggtcggagaaggacgccgagctgctgtacgaggccagggagtccaccagaggtggagggaggatgacgagggagcaggtgtgtatcatgcaaagcaactgcggctcgataatgttatgttatcatccgtaatctgatttatttgttgtttgatttgcaatgaagtacggagcacttaggcggaagattggtggcacctaccaggatttcttcaagtcatatgttgaaggtatcttatctacacctttcccgtgagaatatttaaacgccaagtgaacaggcccgtatattgcctactggtattcagattagcactgcatcactgaataatccgtgtagttttttttggaaacaagtgtagtatgatgaaagattgaaagtagtgtccactgaaaatatattatcgcacacatccttttttattcctaacaagagagaatatcaagttatcgacgattgacgaccaaccaaattggtttcgaatttcagtcgacgggcaatacgtggaggagggttgggtcgacaagacctgcaagatctgcaagaaggacacaaggggggagccgaggcaggttgacaagctaggaagatacgctcacgttgcgtgcctggagaaccccaaaccgacgaactttttcgccaaaatcttcnnnnnnatgaactatgctgcgtccgtcgaaagcgttgtctcgctcgctcgtggttagagagtacaatatataggtagtgttgttgtggcttca

SEQ ID NO: 124 - DNA, 754 bp, Zea mays
tgtttctccagtctttgtcttctgaacttgcatgcattcttatgtcagactggactaattaaactgagtttaccttgtgtaacacaaaggttgaccttacaagaacatttgtttggtaaaccaggtatctttacaatgcacctactatagtctataagtactgcataataacagttgaagcaaccatttgaatgcctaatgaaatgatgtgtattattttaaatcatggtgttatctccttttaaacaggcgacggctaggcgttagcccgagctgcactgcctagcacctaggcgttgtgtctaggcttgtttcagcgttaaggtgttgggcttaatctatgcatgaaaatgggacagattttagatggagaaagatgaactgtggtcatattatttatcgatgcatggtaacattagttctgatgtgctatcttcagaaagtgcaaatgcctagcatcagctaggcgctaggcagagcctggctgcctgcctagcaccgaacacttttttaacattttgtattatgctaaatgaccccctacccctgcagttggcttgtggctactacgacaatcacttccacaataggttatggtgtgaagcactaccgaatcaaaccaaggagtcggtgactgttgctgcaggctattcatggattaatttgctgctgctgatgacatacatcaagtgacttactgacttgtatcaagcatgctgttgtttcttaagcttcgtcaacttcccaatttgca

SEQ ID NO: 125 - DNA, 577 bp, Zea mays
cagcacaaggtattagtgtgtgtgtgtcaacttttgtttctagctagctagttccttaatgaaaacaattggcagccttttgtactatattctcgatcggcgcatatgctcatatcatttttctcctcaactagggctctgaggattccaaggataacggcgacacgaagcaagcagacctgcggagccggggcctgtgccttgtcccagtggcgagcacatacacggtggcggccgagacaccggagttctggcaccctacatttggaggcacattccggtagacaaacaaacaacctcatgcaaagagtgttctgaagctaaagcaaagcaacaacctggctcatctgttcttatcaaactaagttttgtacacagatggtgatgatcgtccagttttagtagcaagctaagtagcctgctgagacggaaagcaagaagtcatgatgccgcccaaagcaatcgaaggatgttttgcaagggaaaagacgtacgagatggatatgataataggtttccggtggtctcttctaagacatgcatacatgctggatcaggtgcagaagagagaatgatt

SEQ ID NO: 126 - DNA, 881 bp, Zea mays
gtccaatgcaaaagcacagggagttctttttgttggggacttgtcatatgcagataactacccatatcatgataatgtgagatgggatacttgggcaagatttgtagaaaggagcgtcgcgtaccagccttggatctggactgctggaaatcatgagatagatttcgctcctgaacttgtatgtagtagtatacttttcatatgctacatggttcaatgggtgtgttggcagttaaaaatatagacatttcactagagccatgctatgagcatttaaatgttcattcagttgcataattttgcagggtgaaacaaagccattcaagccattcagccataggtaccccacaccctacaaggcttctggtagcacggcaccttattggtattccatcaaaagggcctctgcctacattattgtcttggcatcgtactcagcatacggtatgtgtatgtacgactgatgttgtgttttagtcattttattgcaagcatgcactatgccatgagtatttgatataaagataatccggtcatatgcttcatttgggtaaaaataatacactgtcattctgtcaatatgcacatatgtgctggttgctactagctagaatggatagggtgatttatctataaataaatgctattgttccttttccaggcaaatacactcctcagtacaagtggcttgaagctgaatttcccaaggtcaacaggagtgaaacgccatggttggttgtcctcatgcatgctccatggtacaatagctataactatcactacatggaaggtgaaaccatgagggtgatgtatgagccatggttcgtgaagtacaaagttgacgttgtgtttgcaggacatgtccacgcctacgaacgcac

SEQ ID NO: 127 - DNA, 759 bp, Zea mays
  misc_feature (238)..(238): n is a, c, g, or t
aagctgcatccatgaaaaagactctctttgatcaggcatgtattatactcgttgtggatatttctaatcaatactttatctttatgcttgcacaaggacctttttcatatgtataagcatgcctgcttgtggtcccatccatggcagacactgctacatgctgtcttatggttttcaatgtttttctttgatatgtcgaatgctgactcagttttatttcgtcgacaggggtacctanatgagcaattttgccaggtggaagacttgcaggatgaagctagtcctaatttcgcggaagaggttgtcactttgtttttcaaggactcggccaggctaatatcaaatgctgaacaggctctgtaagtaaaacatgcagagcaagggagaagtgatttcttgttcagaaaataaagaaaatatgatgcaagcagaagtgttggacttgactgattaaaattttgtgacactgctgcagggaaaaataccccaaagatttcaatagatgggatgcatacatgcagcagctaaaaggcagctgttccaggtatcttggtgctgcatctggatattttttttagttgcatacgatccattctgtgtgactgattcattacagcacacagaccttactttatagttctgtgaacaatgaggactactgagtttaactactgaacatcctgcaaagctgacactatcatttttgaaacaactactgcatgcttcaccttcttaaaaaaagtggggaaaaaaactgaa

SEQ ID NO: 128 - DNA, 389 bp, Zea mays
ggcgcctgcatcatatatgtgcagtacagcagcaggtatgcatatgcatcagtcaaagaaatacgaccggcatgaacctactgccactgatctactcctataagtcagcattgttaccgatgaagaagccgtagttgatggcgtggttgatggcccagccgcccaccgccgccacgatgatgtacatgcagaagttgagcaccaggaggaggccggccaccggcctcatgcccgcgttcgccatggattgttgtcctaacggactactgatcgtcgatggccgacgagctcgactgaaaacgaattgagctcgatcgctagctcctagctagctacagcttgctagctctacgcctctctacgtacgatttctgctggcttgaggcgtc

SEQ ID NO: 129 - DNA, 255 bp, Zea mays
gttctttttatgtggctggtacattggtctgttgggtttcaactctaatactccaacttgcttgggactataaggctttgttgttgttgttggtggtggtacgttggtccgtgctgcacttaccttctgttgtgtatttacagtgtgctgcccactctcaagatgaagtcaggggaaaagcagttagattggttgcaaaaaggctctatgatctaacatatgcaacagaaaaaatagagcagtttgccatagaga

SEQ ID NO: 130 - DNA, 722 bp, Zea mays
  misc_feature (560)..(560): n is a, c, g, or t
tgatcctgtatctgttgaccgtgcggtggtcgcgctgaaggccactgacggtattgtggaggaggctacagaggaggagctaatggatgcaacggcgcttgctgaccgcactgggatgtttgcttgcccacatactggggttgcacttgctgctttgtttaagcttcagggtcagcgtataattggccctaatgaccgcactgtggttgttagcacagctcatgggctgaagttcacgcagtcaaagattgactaccatgacaaaaacatcaaagacatggtttgccagtatgctaatccaccgatcagtgtgaaggctgactttggttctgtgatggatgttctccaaaaaaatctcaatggtaagatataaagttatatgattaattaaccctccaaactgtttttttttgttttttcgttccaggaattttattcctgagtctttcaactttgtttggtgaacatggtatggtgctaaaatctaaacctaataccttgtagtactagttctggaggctcttttggttgtaggtcgaatggaatagagctaacacacnccaaaagagctcccagaactagtactacaaggtattaggtctagattttagcacctaccatgtgtcccaaacaaagttgaaagactcaggaataaaattcctggaacgaaaaaaccaaaaaaaacagtttggagggttaattaatatataactttatatctt

SEQ ID NO: 131 - DNA, 704 bp, Zea mays
  misc_feature (683)..(686): n is a, c, g, or t
ctgaggactctgtaagtatagccataaacttctgttattattttttagtttatacagacctttaaatttcctttttacaatgcaataatatacagttctcttgcgtgtttgaggaaaaactatagtgatatattaatgcttttgcaagaatataaattggcatttgttggttagattagagtggcaatttgatattctagtatttgaccagttgctacatccatgacacaatgattgacacctagctatccacttgcagtgtactctccaataacttattcagtggatcaatacctaaggaattgagtgctcttacaatgttggagatactggatttaagcaacaacaacttgagtggggaagttccacaagagattgcagaaatgcaatcacttaggcagttgtaggtttctgagtgattaattttgtgaaagttttcatcagcaataagtacaatgtcattatattttgtcgcttctgcagattgctttccaataactgctttcagtggcctctgacccaacattcctatgggaactacgatcaggaaaatgatttcaatatttatgataatcttgggaggggtaatatgaatcgaagagctgagaatgggtaagagaaaaaaatagttctataaatttccttctaagttatcacccatttttcttcttttttggaaattnnnnattatatgcagatcctga

SEQ ID NO: 132 - DNA, 739 bp, Zea mays
catggacgcgaagcagcatttgctgccgcagttctgggagataatgccctaatggagaaagcatggcaggacacaggaatgttagctgaggctgttttacatgctcaggtaggttatttgtttaattgaattctctcatgctcaaccctactttatgagctgcataccattatggttttgtccaataacacatttccgtgtcttcaaattttcatccatacttccatcgtcgttattgcattgtccaccattttttattgggccgaacttgtccacactttgtatgattttgatatgctcactagcctaataaactctataggcccatggtcgtccatcattaaggaacttggttataacatggaacaagatgctacagaaggaactggatcacacaccgactgtaaaaaccgatgccgccgccgcattcttggcttctcttgaggatccaaagctcacaagcttgggggaaactgagaaaaagcctcccattgaaatacttcctcctgggatgcctcccctatctgcacctccgatcgtgattaaaaaggctggagcaaaacctggtctccctaatgcagcaaaggcgtcaaatgggcctataggtgctcctatggctcaaggtgctcccatggctcatggtactcctggggcacaaggggtccccatgaatcaagttcctccatcacagggcagtcatgacgaggcaaggccatcagatgctacagtggctgatgca

SEQ ID NO: 133 - DNA, 604 bp, Zea mays
gaaagaaaatataaattcaactacttcaagttgatcgccaaacgtaggcaaactttgctaggaatggtcaaggtaacgcaaaaggtgcacaactaaagtctagcaaaggtaaacttggggatgctagataagaggcaacatatgctctactattgcattgatatggataaaaaatgaccagaagatgccctctacagctgcatattttcaccaatgtaaaaaaataatcagtatggcaaatgctacagagatacctagccaatatcctgtcccccgtaaacaattaataaagaaccacctatcccactagcgctcttcagaattgtacttattccaagcctcctgcacaatcgatcacgagtgaaaaagttagcctccctttatcgcaacaagtaacttgatatattcggaaagacagttatcaaattatcaacacaagacttgctttcgctagtcattagcaacaggcatacttccaaagattttacgaaaggcgtggccagagacgtactcgcaatttagaatgagcaagcattttaaaacgcagtattactagatctgttaccagtctctcacggatgccaggttcgtacagattactaat

SEQ ID NO: 134 - DNA, 767 bp, Zea mays
cgagggagcgactctgctcccggcggcaagcctgccgccgacaaagggtactacattgaagcccaccatctttgtcgatgtcactgtaagttaaacgacaagcacagcacaacgaaaaatacatacgattagaaacaatggcgagtcactgacgcatgctcatctcatcatctggatggattgaccaggaggacatgaagatcgcgcaggaagagatcttcggccccgtcatgtccctcatgaagttcaagtacgtacaccatacactaacatcgaaattgatattgttcatgcctcttgcattctgagtttctgatgactgacggccgttgtttgcacatttcttcttccgccatgcatccccattccccaggacggttgatgaggtgatcgagaaggccaactgcaccaggtacgggctcgccgccgggatcgtgaccaagagcctggacgtcgccaaccgggtgtcccggtcggtgcgcgccggcaccgtgtgggtgaactgctacttcgccttcgacccggacgcgcccttcggcgggtacaagatgagcggcttcggccgggaccaggggctggcagccatggacaagtacctgcaggtcaagagcgtcatcaccgcgctcccggactcgccatggtactgagttgagccagggaccgaccgatggaaccccatcgatctcttcttgtgcagtgtacatgcgtcatgcgtgcgtgctcacacagctgggttgctgctttgtgcttgtgttcgtctctggttt

SEQ ID NO: 135 - DNA, 555 bp, Zea mays
acagttgaacatgataaatttatagcaaacatatgtgaatttggtcgcgtttgagaaaaaagtgaatttggtatccaggctaatgcttcttagtttcttaattttgttatctattttttttcaggttgaggatgctgttaatcgtgtcacagataatacaaatgcagcacatgaattggatattttgtcattacgcacgcagcttggttctttaactacagttaggagcttacgcttttgtctccattgagtattattaatgatacccattcgtgatatttgatctgaagagttcatttgatctttcagattctgctttgtgttgatgttgctgcaacatctgctaagtcagttgatatgtgccgatttcttctcgatgaggtaagcattatcagttgtgcattctgcatctccacactcttgcagatattgttggaaacaatcgctgctatatcatatttttgtgtgtatggtttctactctgtaccgatacaggcattatctagtgcacaattagattgggttgctgctaattttactcttgctttggtgctt

SEQ ID NO: 136 - DNA, 878 bp, Zea mays
atgagctgtgcgaggagatggagactgcaggtaataatgcttttatggctgctgcccagtcaatgaagggggtgactgacggcatgaggaagaggaagagctgcggatatgaaggtgcagcagctgtgaagtttgtgaagtacaaggttgaagacagctcgctgaaggactacctgtcagcagctaatggcataagcagtggaagtgaggtagagaacccgcagtctgatgatgagatggaagaatcaaactgatgcataggaacgagatcatccgctaatgcttactttggacagttgctttttgggagttccttggacagccgcctttttttggagccaggaccagtcttgtttcagctgccgcttgtttaatgatgccgtgcagaatctgcaagacggccccatctccagctaggcattcggatgtagtagtaaccgttagctgaccatacttgacatggagcatatgatgtttgctatgggccatatcgtgctctctgttgttaatcctctaaagggttctctaccctaaatatagaggatcaaacggttctctacgctctccagcaacgtcctctaaacggtcctttaaatttagaggacgctgctggattctctatatatagagtttctctaaacgatcctttattcatttgactactttaaataaccggtttagcaaaactaaaatatgtacaatatatttgagagtatgataaatacgtatgtacaaaaaataaaattaaaaaatgtctttaatatgagtatttgagtatagagaacgtgatttagagaacgttgttggagagaaaatgagatatagaggatgaaatcttttagaagagactgtaaatgacatatatagaggatgaatat

SEQ ID NO: 137 - DNA, 458 bp, Zea mays
gtaatataacatatactaagtgttgagaaaatctgttacggctgttataggaggatgaagctacaaagtgtagcttgtgtgggtcaatcttccacaagcaatgctttagaaagatcagtgtttgtccttgtggtaaggcttctaatggcaggaagatcgcggcgcttgagcaagctgtacatgatggcacgggcatgccatcaacggaatctattcagcctccacctctctcttcatcttatgggtttttctctgatattctctccaaagcaaggccggacaagcttctgaggccgagaaatagcagtcctgtgatccttatgggttctttgccagatatgtctatatgagctgagttgtatttttgtgttccattgattgtgtatttttgtacacggtaggaaagacgctcctagttttgaatgaatgtaatcctgcagtgttattacttgaggtgt

SEQ ID NO: 138 - DNA, 466 bp, Zea mays
agtcgtagctcagtctcggagctcctacttgccctgaaggatccacggtcattgtcagaatcaagaaacaataccttaaaaaggaaatcacaggaatcagcaggttcagctctagttcccggcaagagaagctccgaaactggatcccgtcgtaactcacatagtggaatgagaaactctctacaaaaaatcagtgaggtgcctgaaggagggaataagaccaggaaatctggtttgcgttcttttatggggtacgtatgataataaaccacatgatatagtcatagcatttgctttatttcaaagttcacttgcagagtctctattatctttgcatctgttctcacgtttagtttgtttgtgcagccttattggtatgggacatgggaatgtagagaagaacattctaaaaccaagggaagatcccctacttgatagcgatgatgagagaccagatagcttcatg

SEQ ID NO: 139 - DNA, 433 bp, Zea mays
cttctccatgtcagtgtccttggatccaatatcctccaacattttctccacagcagtatgactagattggcattctacagcatttgaagcaaggcatttaggagtaatattggtgagtgagtggtcaccatcactagtacttacatctgatttagatattgctagacttgatacaaaatctgaaggagttttgctggtaatgctcatttttatgtctttcttagataaaaccagtgacttggatgctggatgggttagctcaccatttttatttgcaacttgattttcatcacttccttttggcatcacatctggtactacaatgtcatcgtcattccaagctgaaggtattgttgagaagtctatgtccagtgatgactccagtgatgataaagcttgtgtttcctttgaaataccaggcttataacggctg

SEQ ID NO: 140 - DNA, 487 bp, Zea mays
gcatcgcaattcgcagccgggggaaaaaaaagttgacgatgctgagtagaggaatgaatcgaaacagtggtaaactagtaggtgctgccgtgctggtgagtggtgaccgatccacagacatgacatcaaatgacccagcaggccagcacatgacataccaggtgcgcgatgacctcgcagtcgctgccggtcctgaagctgtggcctgcgccggcgaggcggctcctgaggtccaggtggttgtagatctcgccgttgacggcgacgaccaccgactggtcctcgttgtagaggggctggtcgccagaggccgggtcgatgatggcgaggcgctggtgggagaggtagcagtcgcccacctgccggaggccgctccagtcggggccccggtgcttcagccgccgcgacagctccagcacccgggacctcttgctgctgcccttggcctcgtcggcgcacccgagcacagcaagtatgccgcacatac

SEQ ID NO: 141 - DNA, 756 bp, Zea mays
agggcagcagcaagaggtcccgggtgctggagctgtcgcggcggctgaagcaccggggccccgactggagcggcctccggcaggtgggcgactgctacctctcccaccagcgcctcgccatcatcgacccggcctctggcgaccagcccctctacaacgaggaccagtcggtggtcgtcgccgtcaacggcgagatctacaaccacctggacctcaggagccgcctcgccggcgcaggccacagcttcaggaccggcagcgactgcgaggtcatcgcgcacctggtatgtcatgtgctggcctgctgggtcatttgatgtcatgtctgtcgatcggtcaccactcaccagcacggcagcacctactagtttaccactgtttcgattcattcctctgctcagtatcgtcattcgtcaacttttttttcccaccggctgcgaattgcgatgcatgtgtagtacgaggagcatggagaagagttcgtggacatgctggacggcgtcttctctttcgtgctgctggacactcgccatggcgaccgcgcgggcagcagcttcttcatggctgctcgcgacgccatcggtgtgacgcccctctacatcggatggggagtcgatggtaacaaccgtctccctctctctctctctctgatgaatggaatagaagctgaacaatatcctaacaatctgggtgtgctgggcagggtcggtgtggatttcgtcggagatgaaggccctgcacgacgagtgtgagcac

SEQ ID NO: 142 - DNA, 410 bp, Zea mays
cgcttcatgcccatgccagatcactctgtcgcggagatgagagaagatcaggcggcggccatagatgatcatcgtcatcatgaagatggcgacagctggcggtcagcctcagccgcgaaaaagaggaggcgtctggatcatccgttggcaacggcggcggcgcttctgatctttgtgcagccagcaccaaaggccgctaccaatgttgcagcatgtgaatcacaaggaggagctgatcatgagacaaaggtaccgcacacgacaaccctaacaagccctagctctagttcccctcttcttctggatcaacgacatgaattggatctagagctaaggcttgggactacaccaaaagttacgtacatacatggcaacgatacacgaagctagctagctagttcgtctgcacg

SEQ ID NO: 143 - DNA, 685 bp, Zea mays
gctctgtacagaagagtgaaccaaatcgaagccctgcggtgctactccaagcctgaaggctcaatgtttatgatggttagtcagagatacacaaaaattcagtatttgaaactttgttacttcccaacataaaggtgttggtattcacaaaaccctgcaggttgagatcaacacttcaattctacatggcgtcgccaacgacatggactttgctcgtgagttgatcaaagaagaatgtgtcctggttctacctggtacagctcactcactccatttgttgcgatattattttatccaacctttaacaggtgcattggtttctctggctgctgagaactgaatgttagtaataggttgacttgacattccagtacaagattcttcatgctggcatgattttaatctactctaaaattgatctgtagtaacagatccaatatcctttttgtttcttgttattttacaggttctgtcattgggttgaagaactgggttaggatatttttcggagcacctgttagtttaatactagaagcttgcgacaggattgagatgttctgccgaaggaggacgctcaagcaaaacaactgaagagctgaaaatgttttgaaaccaggaagtagggtttttgttagcccagtaacatatatatacagcttcgatttattttccaatgcacattctg

SEQ ID NO: 144 - DNA, 591 bp, Zea mays
tcgtgcagtgggaaagaaaccttccctatcggttctggtttctttgataattgatcaaccattactgcaagaatgagttctctggtaggttcacactgtaaacaaactccggtgttcttccctgtaactgttgagatatccaacctgccgccgtcactgatgaacatatctgattgattcttggtgaaccgtacatacacattttctttttgaacagcagatggtaaattcttgatgtctacaatttgtaaatataactgcaaaggatagagatgttcaagtattaatttagtacagacaccttggcagaagaattgcttgcttcagagtttacggaattatcatatcagttatgtatccagaaacagctgtttataatagtcatcactcatgagtatagtatggcatgcaacattttgatcatattatcagttcagatctttcaagctagaatatttactttgcagtaattgttacataaagaagagagaatcatacctctacaactgttatatcaagggcattaagatctttctgagctttgcagatgtcagacccagtaccaacctcagcactgaatgtctgaggagt

SEQ ID NO: 145 - DNA, 401 bp, Zea mays
  misc_feature (388)..(398): n is a, c, g, or t
taaaaactaagagtcaacatagtaaatgaaaatatttatgttggcatatattatatacatggacacagtgatccagccgctatgtagtgtggtttgaactacacaactaaagtagctaaaagaatgcaagatagggttgcgcgaaccggctaggtggcactatattggtctccatgatccaagatatgctttctgggtaccatggaactctttctcctacaatcaacttgagatatcgagggcaagtgggtcgttcaaagatgtgttaatgttgatattggtgccaatcgatctactgatggtggcgatgatgaaagtgttggcatctttcgtcttcaggagcaaccccgttcgataagaaaatcgttgattctaaagatctcaacgnnnnnnnnnnngac

SEQ ID NO: 146 - DNA, 419 bp, Zea mays
tgacgcccctggtcacgacatggggcaataccgagcgtcgcaacgggagccctagacacgccactaccactacccgtgaagaacaaccaccgccagataggtcgcagcaggtcgcgcagatccatcaggacctgatggccacacagcttggggtgaccgacgacgctgtcgtcgtcgtctcacatgccgagaacctcgaggtcaaagtttctggatctgagtctaatgtggttgttcccaacataggggtcaccatgcacggcagcaacgaggtcacagagaacagcggccgcagcagccatgacaataattctgtgatcatcatcatcgacgatgacaatgacgacgacgcaaatcctgccatcaccgtcgcagcagggtctcctggagctcctggtctagctcctgatgcagcggca

SEQ ID NO: 147 - DNA, 804 bp, Zea mays
tgtcgtaaatgcagtgcttgtgtccttttgttttaaagattgaatttgttaatgtgtattgttgatttttttttgttttgttttggtccacatgttgttgtttaaatgatttataaaagggtgctaatttatgtgtatttggtcatccatattgagtataaatacaaaaaatatcctgatattgcattagatgcttacgatgaactaaattttgttattaggattattttgtctcaccatagtacataaaaccatattacttgtcacatatgttcattatgcttctatatacttttttttggtcacagatgtggaatgtgcaatggtaaaggaatgttaccttgtatagcatgtggatcacgtggattagtgacatgcaacacatgtactggatatggttccctacttgcacaaagcatggctcatgttcaatggtaatcttgagctcatttttccaagtatttatatcggtttctgtttctaagttgaggagattgttgaacaacataaaaatccaaaacatctgaatgcaaaagttgaagtttagtttggtaatttaaaaattcattcttatcgtttaataatcaagtaaatttctggggtaacaaacctatagcgagcagttaggaagttgcagatattgtcagttgatgctttctttgtgaatatatgtttatcgttacatcataaaatataatttattgagagtctgagactcgttggtgttgaggacttgaggcaacaaattcgttagtcttcctatgtatgctctataacatgtcaagtcaaataccaacagatatt

SEQ ID NO: 148 - DNA, 804 bp, Zea mays
tcctcatggagtgctgaagctccagccccacaccagaggctgctggaattggacacaacctccataatagcatggatacacgaccttgcagcatcgaacacacaagcattcctgtgtttccgtaactcccaagcatcagcggaattctacaaaatattcaagattgtgctaagctgtggggtttggctggagcaaaagctcttaaaaggctctggccttaatctaggatttagttcccctgtactttgaggtttggtttttggtgttttagctcagttttttttagtttctgctgccattcattttggcggcttgtacttggtctattttagagctttctgttcttaatatagtgatacacaattctcctgcgtattcgagaagaaaaataactcccaagcgaccaagatgatcaacaaattgagacaatgagacctttcttaaactcctttgaaactctctttgacagtccggctccaccaaccggagaaactattagtgtcaggtaggggggcagcagcctgacagaccaaccctatgcaaaactgatgcccagacctgtctagagaacatacaggaaactaggatgtgctgaattgtttcttcagcttgatcacataacgggcatgctcaggatggggcagtccattttcattaattgaaaacataaacattttcctttctttttgaaactaataaattaaaatgggttgcttttaggtgatggattttgagtggtacagcaatgcctcaaaattggttggaagatttaaataaagtggcaaattattctatggggtttta

SEQ ID NO: 149 - DNA, 354 bp, Zea mays
gaagctgtggttcgaatggagcgagcactgtactacggaagacttcaaacttggtcaaattgatattgatgactctggggacgaactgttggatactaaagcaatccgcaaggatatcaaagcgctgtatagaaggtgagcttccctccttcccttactcacaattgttttgcatgctagttgcctcatctagtgcctttgtaggtaggtaacaagctttgtggccactggacaccatgtacatattgcagtgctgaccgtcgagctgatccttaaactgaagaaagaggtgacggtgggtggctctacgatcatgaaaacgatgtttcctgttccatttcattgtatggcagc

SEQ ID NO: 150 - DNA, 377 bp, Zea mays
gaggaataaattataatgagaggacttaacatctttggtacagcactggttattgttaagtggaagcagtaagctctcaatgcagaaaattgtcaaaattaaaattatgttttatcttgtagttactgttgacattgcatagtacgtttgatcaattgcagggcttcgagctctcctgtggcttcgttaagcaatgggagctgatacgtgaatcacgtgatgagggtttcaaggatggagtaggtattgatgttgctatcagtatggtttctgtagtcccatggaacaatggtccatggatagtctgacaaccttctctacagaactttggaacattcttctctcatgtatgttgtctgtcttcagttaacatgcca

SEQ ID NO: 151 - DNA, 911 bp, Zea mays
attttgaagatttgctcgcacaactgctggtggtattacatctatcttgcatggccattattttgatgttcccattgtgagtgtagatgctagcagtcagagctactatttgttgagcactaattttagttgaagattttgtcaattaaagggacaatggggcacttcctagcttcgaaagtcaacaagccatatgcagttgaagaaaaagggtaaatgctggggttacaatcttgtaacagaagactgagcaaaccaaacatatcaatgatcctgaaatggaaacacaacctttaaaaaaaactcaggagttacatagcatactacatcacattcatgtcgtccattcttgctgctcacatacatatacaaacccgcttcctatgtcgtgataaatactagtgcagcatggttcaccttcatctcgtttggaggttggaagtagtcttttcatcggggctgttatgctgcagtcttccaaattttgtgttgtttgtctatgatcaaccattttttgttttgtacaagtattgaatcccaaggctccaattttatatgttactatctggtgccgttattttttctcttacaaaacatttttgtgtctttatcctgacatatatgcatgtatgttacattatttcttcccagctgtttcagttgagcaagctgaagctattgctgacctcccaaaaggtgacatgtggtcggtgcacaagtttggtggcacatgcatggggacctctgagagaattcacaatgttgctgatatagtccttcgtgatccttctgagaggaaactggtagttgtatctgcaatgtctaaggttaccgacatgatgtataatcttgtaaacaaggctcaatcaagggatgattcttacattgctgtactggatgaagtttttgat

SEQ ID NO: 152 - DNA, 255 bp, Zea mays
aattcggcacgaggcccttccagcccggcagtcccaccgtccccgtcctcccactcccccctcgcaaccatttcaaatcgctctctcgacccaactgtactcgccgtcgtcaattcatcatccatccccatccccccatcgactcccagatctctatccccctctccctgtcctcactctctcgagcgcgttgtggcggatgtcctcgtcggcgcgtttcgatctccgggcctcgtcgcatcagttgcagccgcc

SEQ ID NO: 153 - DNA, 843 bp, Zea mays
gcttgaggttggccttgtcgccttctggacgagactggctgttcctttttgttcaacagcacttcgatagttcgatatcctgagtgatcgccaaagggactcgaaacggttatcagattttgattcctttttttattcttttgtttggatcatcgataaaaaaaaagagttacaatagggaagcgtacttggagctgaaggagttcatcagccgcacctccctgaacgacgccgagattttctgcgaaaagctcatgcgcgagtctccaaggctcaagggactaggcaagtctagttcttttttaaccacacactgttttactttcttgatcctttgatgaaacagtgtatgaaattactgaggcatgtgtggtttcgtctcttggaaacaaacagccttgaggattctggaggtgcctactgatcagagagcattcgtatctgatgtcatattctaattaaacttagttttcacaatctgtgttccaaagggattctcactttctcagatgaattgatgcgactgcaggtccgatctgcatatgtgaagaacgagttcgagtgggacaatttgaaggaattatccttcaaggtacgttgaatttttattaatcttctgctccatctactatatgggtctattacttgctaataataaatttcgacagactaaacttcttaaagtgtctcagttatgacccgcagtattccataaattttttggtcagtaaaattgtgtcaaatattaccaccaaaagatagatcgtagtcagtaagaatttcagaaaaagcaacagtactatatatttagcgttgtaattttctatgctcttttgtctccac

SEQ ID NO: 154 - DNA, 245 bp, Zea mays
tctcacctgatgagcagcagtgcacgtacgtcattgccggctctgcttgtgcttgctaccagatattttgtacgcacatcattgccggctctactttgctaccagatatttttatgcagtgccaaagcctgtcctgagtacacgtggtcgcgtatcatttgtttcccccctcctcttccgtgctcttttctgcttcagctcgagatgtgtgtacacactacacagtcgcacatgtacgtagagaa

SEQ ID NO: 155 - DNA, 372 bp, Zea mays
ccttcaatctcgcgttgttgctggtcatctcggattttaacgatgaactggagatctgatcgacgaaccggaagcgcggtaacgaagctctcgccaaactggtcacgatgatgttttgggttgccccgaaagatgaacaaacggagagaaattcctcaccggagttgtcgctatagtttgggcaagagtcaaacttcgacgagctggttatggaggcttatggctgcgagcttagttggcggcggactgctatggcttggatggcggcggcgctgctgcgaggctattgaggatgagtctataattgatggcttgaggtgttatggtggcatatatttatataggctgggagatgggtggttacatgataga

SEQ ID NO: 156 - DNA, 504 bp, Zea mays
ggatcatcctggccgattcttccaggttttgggaatgacgaggtcctgccctcaagtccagttgactggtgaagcctttgattggttgatggacggctgctgctgctggtggtcgggactaggtggctactgcgatttgggtcgctgggatctccagaacctcggctgcttgacagaagagccttccttgatgtgcttccattccgagatgatgaatgctttcttttatctgaatcaacagcctggaatacgttgtgaaaagtgttatgcgatgagctctttagtcaatcaccgtgggattgtaaataaattctttggtaaccaccatgccctgaatatgactaaagacggaggaacaaagaaattcaattacttcaagcacccatattatctgctgactagagctacatattttcaaggcactaaaagaaaaatttagggctaagggctagtttgggaacaccattttcccaagggattcctattttcccaagggaaaatgaa

SEQ ID NO: 157 - DNA, 690 bp, Zea mays
  misc_feature (383)..(384): n is a, c, g, or t
ggccactgcattcaatagccactcaaaggccagaaagaaaaatgctgccaaaaactcatgccacacaaggtccgtcacggagcagatacttcaaacgaaatttcttggtaagttcaagcgcaagagaggtagaagaaacaggcctagctataagcatgataattccagtgagcacggggaagcagaatacaagttatctgatagatctgaatctgacggaaatggctgcttgctgctacttcaggcacttgaacaggatctttcacctccaaagttgtccgctgaacatgatggggaggccttaactgacctgaaggaagatttcgaaatgagcgggggggatgctgagttaagtcggtgtactgctttctcggaactggtanntgtacttgagatatattttgatgcaattgaaaaaacattgtgcttcaccttttgcccatgctcttgtcatgtacgcatccaacaacactcccttgtccttggctctatctatgcatccacctttcactcttcttttcatccttgtaatagaaaacngnnngaggtagcagacaaagaaatggaannnnnnntagcagtcgtnnnnnntgttcgnnnnnnnnnnnnnnnnnnnnnnnnnnnnnnnnnnnncccannnnnnnnnnngactnnnnnnnnnnnnnnnnnnnnnnnnnnnnnnnnnnttat

SEQ ID NO: 158 - DNA, 578 bp, Zea mays
ggggcatcgggcagctcttcccgatgatgccggtgagccgcctcaacgaggagccgactctcaagggcacgctggtggacatcacctgcgacagtgacgggaagatcgagaacttcatcagccgcgaaaacacattgccgctgcacctgttcgagccgagcctcggtggctactacgtggccgtactcgtctccggggcgtaccaggaggccattgctggcaagcacaacctgttcagtggcccaaccctcgttcgtgtcaatagtagcactacaaaagatggcggcttcgagattgaacgcgttgacctgggcccagctgcggatgagatcatcggcagcatgaggtacagcgtcgaggaagacattatcaccgtcatcgagaagaaagccaaggggacagaagaagaagaacgtgtgtgggcgtgggagatggtggagccactcatgaccaaagcgctcaccaccatgccctacctcaacgaatacaaggtccctgaacctgaacctgaacctgaacccatcccttgaacttctgcactgcagagtctccggatttggctgcgactgcgatcatat

SEQ ID NO: 159 - DNA, 344 bp, Zea mays
cggcagagggacggcgggcagagcgacgacgggcaggcggtcggggccgagctgtgcccgacggtggcgttcgaggggcgtctcgttgctgcttagggaagaaggttggaaatcgatctcagtgcgtggctccggcgttggagatgtaccagtgtccgctggcgtctcaggcagccgcgtgctcgctcgggctccgaacagccaaatctccggtgagggtggaggcgagccttcacatgcctgcacgatggagatgtgcccgcttgcgtcgaagttggagatgcagcaagccgaagtcaaagttcagactccggggaggagcgcgtggctgagatgaagccaac

SEQ ID NO: 160 - DNA, 744 bp, Zea mays
tactatggacatatctctgcattattataattgtcttctcattgttgaactggctctcaaggcacttagttgttaacgtttctcctctctcttttttgcctgaatgttggaatatctttatgtgtttttatgcttgatgcatggcaatttgtattaggttgccaggttggtcaggaagttcttgtatggacttccagaaatgctaactatttgataattcttttcactcttcctgattcagttgatgaagtagatgaactataatgcttgtggtaaatatttggaaaatcatctaagttagcccttttgttgatacttggtagtgctgcttcatcaacatgttgagaccatattattccatttccagcattgagttatattaccaagtaacttgagatcctttttaggtttagcggctaaatattatagttatttggttttctgtttatcacagtcgatgctttcccatcttttaaattactatggacatacatagtttacgtcattaggtgtatgtatatgtatactgaactgacagaagaatctacaggtccagaagttacacgcttggctgctatggctgcaaaatataaggttttcttggtcatgggggtgattgaaagggaaggttacaccctttattgttccgtgcttttctttgaccctcttggccgttacctgggtaagcaccggaagctcatgcctacagcattagagagaattatatggggatttggggatgga

SEQ ID NO: 161 - DNA, 702 bp, Zea mays
gctgcttcggcgatgcaggagttctgtaattgagagagctgctagtagctgcaaatatcttggacaaggtaataatttcagagctcaatgttcctactaaaccatatggcacttatcattggatggtatcctttgagcgcgaacttgtttaaccatgtttacttaccattactgcttgctcaaattgtttccaaattagtctaaaacgacattgatgtgtttcaggagagggttttaagatgccatgaaatgattcaagagaagattactgcgggaagcattgtcctaaagtctgctggatcatcaatctcctctgtgccacaaagcccaataggtgtcctggacgctgcagcctgtctgagtcaacaaagcgatgacgctactgtcgggtctcctgcagtatgttaccatagttcttccacaagcaagaggagaaggatcactagacgtctactctaattgtggtacgcttcaggtgtgctcctcaccgctctaggagtttttgattggttcaaacatcttaaatttagtttggccgctggaggattatggtttagtcaagtagttgctgaatggaacaacaaaacacgcacactacttggtccataaagacaaaaaaaataacaggcagcgtcccgcgagccagcgctgcaatccagttcatgcaagaccctagagtccagggggagtgctggtgtaggt

SEQ ID NO: 162 - DNA, 405 bp, Zea mays
tagctaatggttagctggactattagatgagacatgagagtgtttggatgtctctaactaaaaataaataatatctaaactattagttgggttattcggatgtcttcagctaattttagcagctaattattagctctagtgcattcaaacagggtctaagtatagactctagaaaaaaaaggaacatatttctatcctgggtttacactgcttattatgtgccatgtgtattgttgtacccgtgccttctcatgactccttaccaaaggatttgaggtgttaactcagattccattcatattgagaccattttgtgcttataggttgctgacttgctgatcagttactgtgtctgaaactattctcctttatgttttgctgcctcattatggaaacacctctgta

SEQ ID NO: 163 - DNA, 437 bp, Zea mays
agttagtgatgataattccaacatgtcctttctaaaaagaatcgctacaagtgacgtgccgctgttgaaagagtatggtgtgaatggagttgttgtcgccctgcttctggctgtagttactcctgtcctgctttccatgatgtttggcaagaagacaaagcaaagggcggtgcgagctgatgtgggtggagaggagggatttgcaatgcggaacaacaggttttcctccttagttgaagtcccatgggagggcgccacgacaatgtcagctctgttcgagatggccagcaaaaagtactctcgacggaggtgtcttggcacgaggaagctaattaagagggaatttatggaatcttctgatggtaggaagtttgagaaactgcaccttggtgaatatgaatgggatacctacactgaggcatttaatcgtgcatgta

SEQ ID NO: 164 - DNA, 792 bp, Zea mays
gttcaagaattttgtgtctagattcttaaatctgaataaacaagttgaatatattaactgcaatgaaaacatatagatgtcatctgccacctatctccacgcacagtctggctcgacatcacctgcccttctcgtgatatctgattactgttttgctagtgctagctatcgatgttgttcggggattagtagtgttctatttcatcctcttaatgtgttcgcatcagatagatatatgttcatatgatttaaggatatcctgtcagcagcttacacatttgtctatctccatgcttcctttctttagttggagactggattattttttgttgggataaacttagtacattatcagagcatcttaactctggatatgggtgcctttaagggatatccaataaacaaaatttgaacacatttggtttgtcgtataggtacaaagccagcacatgcttgggcaggtttgatcagtttcactaggaactgttcactaattgactgtctcagtaatacaattgaatagcctgcaactcagttagccatgactttcttcgtcatagtttctttagctgaattattttgcacatattacatggatgtgcactacccatgttgttcaatatcccacatcaatatcgaattgagtatgatgactgatgacaactaccagttattgtttcttgaattggcaacaacttggtctgtgagtcattcccactttgcccctacccttgctgagtagtgtcatttcttttaggcataccttatagatgaatgattttttacattact

SEQ ID NO: 165 - DNA, 255 bp, Zea mays
ggcagctggaactgaaggtggcgggagccgagtgatccggcggtgagctgagcggggcgggatggcggcggagcaggggcggtcgctggcggagacgcccacctggtccgtggcaaccgtcaccacgctcatggtcgctgcctgcttcctcgtcgagcgctccctctcgcgcttcgccaagtggctgcgcaagaccaagcggaaggccatgctcgccgcgctcgagaagatccgcgaagagctgatgctgctcgg

SEQ ID NO: 166 - DNA, 516 bp, Zea mays
gcaccgacaacgacgcggacgccgcggcgtccgaggaccccgcggtcgagctccgcgaggcggaggttctgccctcggccgatgccatctctagcttccctgccgccaagcgccgcgtggtcaaccggccgcatgcgtccatcctcgcgctgctcgaggccgaacgatctgcttgttccggcgatgtccctaccgtggcactgccggcgctagagaacatctcgcacgggcagctgcaggtgctgtctggggtgcttccggaccacccatcccttgcgacagaccccgacaagccgtccttgtatgtgtgcacgccgcctcccctcatggagggtcacggggtgcccaagcagttccagggtcgcttccatgtggtgcccaagcactcaggtttgcttttcgtctaaacaaggatagctgagctgtgctggaaaatgtgctaatttaagtaacctatgtattttggtttgtaatttgtagattggttctccccagggacagttcataggctggaaa

SEQ ID NO: 167 - DNA, 702 bp, Zea mays
  misc_feature (690)..(690): n is a, c, g, or t
atgcttcacctagtattcatagcctctccccttctgctttccatttttttttgtaactgaccacttgtcaatcggcagccacttttgaggatctttgagccaattctgaagaatgccagcaaagagttgcttcatggaagccacaccagatctgtttcaatctcaactccttccaatagcgggataatgaaatttgcaaagaaacaattaacttgccttgggtgcaaagctgttatcaggtgacacatgcactacattgcacagatgcaataactatgcaaccagtgcctataaactcttaaccgtttgcgctgcattttcaccctgcagtggtgccagtcagacactttgctcacattgcaagggaagagaagcagagttgtactgcaaaacagtagcaaatggtacgtgctgtacactctgaaatgcatctacattctgcctttggaagcactatgcccctgagctcaccactgacagatcaaaattttgatttccgcagtttctgacctggagatgctctttgggaggctatggacacaatgccaggagtgccaaggatctctgcaccaggacgtcctctgcacaaggttttctgttttcccctcaatccaacacctgctcattacttggttccagtcacaagcttaaaactcctcgtatgcaatcgaaccactgcagtcgggattnccccattttcta

SEQ ID NO: 168 - DNA, 255 bp, Zea mays
  misc_feature (27)..(53): n is a, c, g, or t
ccgccgccgccgggcaccgctcctccnnnnnnnnnnnnnnnnnnnnnnnnnnntggctgccacctctccctccggcgaaaatggtcgaagcgtcgcctccgcgctccaccaaggcatcgctcgctcctcccgttccgtctacacggttcgccctcccgtatctgttgctattactcctgagtttcggatgctgcttttgctcgattttctagccgttggctgtcgcagattggttttgtgatggcagattacaag

SEQ ID NO: 169 - DNA, 401 bp, Zea mays
attttcactctgccatgcttggtattatttgttgtgctgctgcggtgcctggttgggatcttgtgaattttgtcttgcccttggagtctagatgttgtttttaattgctcttttgggtgggcgccgttgtccagaagaatcagctaaagttgatgatgttttggttggcgtcagactatgtgctgtccggatagttggctgttaaggatgtgaaaacgttcttgttagtttgcatatcgccttatatgagatgatgttagtatgtttcttagctggttatggtaaaagtcagtcagttttttttccttttcagagctttctcgatctggaaggattggctgctgaaatcgccatttcttgctgcgtgcttgcctggtctggttgtaacagtgatcgtgtac

SEQ ID NO: 170 - DNA, 731 bp, Zea mays
cgcctacgacgacgccacctccaccttcacctgcaaccacaccgattacaccatcaccttctgccccaaatccactccaaccaggtccttgcccaaatcagatcattccttcatctagttccttcacttccagtttcaacttcagttgcaatttcgagctgtaaggtattgtttgatatagaattcgtgagaatgacgaggtcttgattcggaattcttaggcttggagaaaagcaaagaagctttgatttacactcttctctttcccatctttttccttgtgtccccactcggcaccttgccaccttgtgtgctgtgtgactagaaaaccacaactgccgagttgggactgcaaggaacacatcagactgatagagggttccacatccgatgcttagattaactttacaagttcatgctttacaagattaagcaaacctggtcttacgcgagcaatatttcttttgacatttttgcagcgacaaatccgaacattcgtctcggaggccaagccacgagcaactggaagattcggtgtggcttgcatccttgaaggcaagtgacgccagtgctctggcaacaacgtcatggtcggcgtccattgtgctccgatctgccttggcaattgcagtggcgaccttgcttgctgcacaacttgcaccagatcatcctatggtcagcttgctatgattcatcatggttgcggttcttgatcggtaggagtcatctaa

SEQ ID NO: 171 - DNA, 392 bp, Zea mays
gcattattcttggatcaacactgctggcgctcggaagtacctgttcagttcctcttttctggtgctatgagataacagcctggggtttggtagtcttatatggtggcacggccttcttcttgagaaggaaagctgcagttgtccttgacgagagcgattactctacccacaatgtgggtctagagatgctggagtccaaaatggaagtgactccagagatgcagcgccgcatcaccgatggtttcaagcagtggatgggttcgtccctcctatcttctgatgacgaggaggaagcctcggatgactacctcgagcacgatgttcctagcccgactgccttaggtcaacaacaccagcatgagcatgatcgagagacctgactgctgtataaa

SEQ ID NO: 172 - DNA, 730 bp, Zea mays
  misc_feature (701)..(710): n is a, c, g, or t
aatgcatgtagttttcagacagttctgtccctgctatatctttttctgttcctttttccatgtctaaattcggctcgaggttataatgctgtacaaggggtctcatgtgcatgcaccggctaaagattgttcaccgggacctcaagagtgcaaattgcctcgttaacaaatactggacagtcaagatatgtgattttgggctctctcgagtaatgtcaaattctgcaatgaatgacaactcatctgcgggaactccagagtggatggctcctgagctcattcgcaacgagccattcacagaaaaatgtgatatcttcagcttcggggtcatcatgtgggagctttgcactctttgtagaccttgggaaggaattccacctgttcagatcgtttactctgttgcgaatgatggggcaagactggaaataccagatgggcctcttggaagcttgatagcagattgctgggcagaaccggagaggcgtccatgctgccaggagatcctgacccgccttcttgattgcgagtataccttgtgctgaggctttgcttctagtctgtatagcggttataggggcatcagacggtctggcttatatatacatcacttaattttccatttcacatacatcattgagggtgtaaattaccatgtattaataagcaaactttttctaaacctctttaatgctattgccccnnnnnnnnnnccccccgaccgcgtgtaccc

SEQ ID NO: 173 - DNA, 255 bp, Zea mays
  misc_feature (1)..(30): n is a, c, g, or t
nnnnnnnnnnnnnnnnnnnnnnnnnnnnnngcatccgccannnnnggtaccctcgtagacggattggcattggctgccccccggccctgttcctcgtcctcctcatcaattcccgtctctcagtcctcccaataatggtggcgtggtgagaaatccttccgtccggccgtcgtcttagaagatgaactgttcctcttgctgcgccgctgccactccatcttctccggcgcttctcgccaggccgcgggggggttt

SEQ ID NO: 174 - DNA, 332 bp, Zea mays
gtcaagtccggcgcctccaaggtggccgtcaacgacgtcgtcttcaagaacatccacggcacctccaacacgccggaggccatcacgctcaactgcgccaacaacctgccctgccagggcgtgcagctcatcaacgtcgacatcaagtacaacaggtccgacaacaagaccatgtccgtctgcaagaacgccatcggcaagtccattggcatggcgaaggagctcgcctgcgtctgaacctacttgcatccatcactcactcttcgtcacctctctctttctcactctcgccagtctttttttaggcctctggcaatctgcgaactttctta

SEQ ID NO: 175 - DNA, 770 bp, Zea mays
aaagatctctgattctgttcgctccaaattaattacatgctcttttctcttttcaggaggcttctcaatggcaactgccaacagttcgtgattagtatacacaagcattatggtaacaaagaatcgattagtcagatttgcaacagcatatatacctggtacagctaaatttatttcacggggtttctttctttcaggaacctcagataaacctgttagtgaaacttgaaaatcatgtatcatgacatgaaaacaacataagaaaacacataaatacattacacaatggtcaatgaaagttaaccttttttggcatcatgtctagactgcaccgaagcttgcttgctttttggctcctcagccctctgatgcctacctaattgagccatggaataatcacgtctagtctgtctttcttgatacttggacatgcaaaatactaccagtagcgcgagaactatgaatagcacaatagcaagaagaacatatccaacagttctgagggttgaaggcttcttcttcttggatgacgtagaattgctatttcgagcagtagatccactagaagagtttgatgctgcattagatggtgaaggagatcctgaaggggatgaagaggacgatggtgtttgtgttggtgagcctgtaagtgaaaaaggtgaggtggagggagctatgctggtattaaaaggattcccatcatttctgtacgcaaaagaagttaatggagtatttgtaactgggccaaatcataaaggggtgaaagacag

SEQ ID NO: 176 - DNA, 255 bp, Zea mays
taaacagcaaagtaacagaaatcaggaacataccattacaaacaccatcacatgtctcctgcatttcgtcataggttcctcggtgcgattcaacttcgagaaaccaacgtagatccgtccggtccgatgccactgcacaattcaggtttaagccttgagaagtgaggacagtttacccgtgcgaaggcgtgcgattcaaggccgctggtgaggaggtcagcgacgtccttcttgaggaacttgaccatcgcttgc

SEQ ID NO: 177 - DNA, 354 bp, Zea mays
atcattgggtgtggtggttatggtctagtctacaaggctgaactacctgatggctgcaagcttgctatcaagaagctcaatgatgaaatgtgcttgatggaaagggagttcactgcagaggttgaagcactgtccatggcacagcacgaccatcttgtgccactgtgggggtactgcatccaggggaactcgaggttcctcatatactcctacatggagaatggcagcctggatgattggcttcacaacagggatgatgatgccagcacatttcttgactggccgacgaggctaaggattgcgcaaggagcaagccgtggcctttcttatatccataatgactgcaaacctcac

SEQ ID NO: 178 - DNA, 519 bp, Zea mays
gtcgtccgcagagagaagcgaaggcgttggccctgccagccaagccagactagtgcaggggtcgtaggaaaaaataaataaataaataaataaattttgacgcagatgagatgggaaatcccagcttgacactttgttagggtattgtgcttgataaaaaatatggcaccaggtgaatctgaggtgccagtaaatcttggatgaattctgccgcacgcatccgaccggatcagcagaaacttgtctccatgccatgatcagggtacctgtgttgtgacttgtgagtgactgtggctgtgactggtgggtgccgttgtcgacgtgggataatgcctgatctgtccagttggatcaaaaaaaggaggacgagtttgcttgtggaagagaaatctgcgaatatctgttcggatgcgttttgcatatgcataaaaggcataaacaaggaaccccttatgcctttttggatttgtcatgttcttccatggtgtacatatagatcagtgtgcgatgagaaatgtg

SEQ ID NO: 179 - DNA, 817 bp, Zea mays
gaagcacatggactcctcccggacttcaagaaccaccgcatagtcaaagtcagcctctcctcctccacggactgtgaatcgtagtagttgtgagatatgaactgcaaattgtaatgccacgctatgctcccctgtgccgtgcagtccttcctctaccagattctgcggggcatcgcctactgccactcgcaccgcgtgctccaccgcgacctgaagccgcagaacctgctgattgaccgccgcaacaacctcttgaagctcgcggactttggactggcgagggcgttcggcatccctgtccggacgttcactcatgaggtacgtttcgcaccctgatgacacgctctcggtctggtagtatgctggcttgaggtgctgcgtgagttcaatccgcggtagctgggaaattgccatgccatgttattgcttattagcctactggaaatgaatgcggtcactcgttcataataaaaaaaatcctaacaaaccactgttacatctgtggttggatcataatccaacagatcagatggtttgcttgcatgcttgcatatagggactgcttatataacagtcgttctctataataaaaggctataaactggctaaatgctgaggtggacgagagagagaagaggataagtggtctgtaaacttacagccagtttttacacgagaatcaaaaaattttgtgagagagacaagtggagtcttgcattaatactgaagagctgaccgccatatgaatcggctaaatggtaggctgtaaagatccttacatccagcggttggctctattattaaaactgctcttatg

SEQ ID NO: 180 - DNA, 611 bp, Zea mays
catgcgccctcctcagctcttccacacaccactatctggatgtcaggtgcgtgcctgctgatcgatcatccatccatcccatgaggccatgatgcatgtgatttatttgtactagcaggatgtgggaggattttgacaagggccatgtcgccggtgctcggaatgttccctactacctctccgtcactccgcatgggaaggagaagaatccccagtttgtggatcaggtttccgcgctctacgccaaggaccaaaacttgattgtggtaatgcctgcctcatactgtgtttacataattaattgatttcatttgccgctaaccaaaccaactaactaacagggttgccgctctggcatccggtccaaactcgccactgcagacctcgtcaacgcggtaagcttaccttaacctagtgctatctcatcttagatttacaactaattgttttttatatatatatttattataagagtgagtttattgtgtacgtgtgctgcatataagcagggcttcaccaacgtgaggaacctacaaggcggatacctttccttgctccgaagcgcaaactagctagcttcctgcttatttgttcgcatgcttgtaataagt

SEQ ID NO: 181 - DNA, 255 bp, Zea mays
  misc_feature (1)..(8): n is a, c, g, or t
nnnnnnnnccggcccggccgcagcttgcctgccacctggtcttgaccctctcctggattaggagagccgcagccagagggacatcaaactgtgctgcctgcgtgtgtgtcttctttcttgcctctcccttcttcaacctttgctagcttgccagggagggagtccatgaattccagcagaagcctcctctcttcgcccctctttgctagctcgtctccaaacttcaggagcagtgctgctttcaagtcctgctcc

SEQ ID NO: 182 - DNA, 353 bp, Zea mays
tcacccaaggatgtttttgttgttatatccgagaaagaatgaggtttatcagatcacagttgacacaaaagcaagctcctgaccagtgaccacacctaatgaagaacaggcagctgcgccgcagcacacacacacacaccgaccagcgcagagagctatttgattcagatgcgcatttggctgattcagatgcgcatttggcagaaggcaatcgctgagcgcgaaggagtaaaagcatgatcagatattcagatcttggatcagtctagactgacaccttaggtcgccctttacaaatatgacttaaaaaggatgtgaacagagtagtacagcacatgctttggaagtttgga

SEQ ID NO: 183 - DNA, 306 bp, Zea mays
aatccgagacatgacgctaaggtcaaggaattgcttgtttcagtgtgcaatgctgcggttgagactttggtgaagaccactcaccatgccctgtacagtactaatggcaacttgggtgcaaatggtagtgtcagtggcaatgccaatggagctggggaagggtgggtggagacggtgtcgagcacgttggcagttcctagcaacaggaagtttgtgcttgatgttacagggagggtcacatttgagacagtgcggtcatttctggagtttgttctttggaagctgcaggatggggcaaggaagggc

SEQ ID NO: 184 - DNA, 814 bp, Zea mays
ctagcttgcatgcctgcagctagagcggatgtatcagatccgtcgtcaacttcctggtacgcatcaggtttcggctgcgcgcattcttgatcaatatcccacgaagcgtcctcttcacaggccggcacctctaaatcaatcacaaatgctcgtttaggatggttgttccgtgaaggttccacgtggcattcgtgtgacactactgattcctcagaattgtcatcatttggtacatcatcattgagatcgatgaagttcctgatgggggcgccacttttcttggtggctccttcagcggcttgtccatctttaggcacatgcttctggcacgaaagaggtaaacatgtagcaccctcttgggctttattacagacaggcagagttccggtaacaatgttatccgaaggcctatccttcttaattgcacagccaacagatgtagaagcagaatctgacaaattccgaatagtcacagctggttcattctggtctttagtatttttcagctctattgcagttcccaatgaatgtccaaaaatcttccctggcttctccacagctgttgggtcagcagacataagtttttttttgagccaagagatcccaagtactgaccccccatctttatcatgctgaaaactggaagcacatctcccatcttgttctacaagaccatcttcctgacaatctgacagtgtctcattcaaattgaagttcttcatacccttgccatgggactgatgatctgaatcttccaacttctgggaattcttttgcttccagttacttgaaactgctacctgatgtcgtgagt

SEQ ID NO: 185 - DNA, 878 bp, Zea mays
tatgttgtctattaaatgttctctttcaagtacatattttaagtgatcataaatgtttgtagtgacaacacaaagtcactgaacaaagttgtgctacatgttaggtcttctgctgttgtcagagaagcccacagtcccatactaaggagccgaacacggtctcgagcaaaggtattacactgtcatgatatggtttttgcttgtgatgatcttatgtcctcttactgatgctggtttgttgaaactggtgacatttaggcggcttcaacaagtatcacatcagttctttatctgaaagatggtgtctccattgctacttctggagctgcagacaagtaagttgcactttcatttttattctgcattttaatttattacccactttcatacttttgtatggtttgcaatagcttattgattttatgatattattgtgaacataatgcacttgcttgacgatgagtgagcctgatatcatctactctctgtttcttgtttttttttaaaattttgtagagtaaccaacgtttatatattttaaatactaatattgtaccttgtaaattgtatgccagtgttgtgaaaatatgggatacacgaaacctgaaagtgccgttctccaacaaaaactctcaagcaggggcccaacctttggtaaacatctctactcactaaagaatgttaaactatcagtacaaaaagcaattgaatcgtgcaattcagagtataatcatgtctatcaaattgagtcaacaattatggaatgttgtgtttccgtagctcaaatctggacaatctctgaacaaacatactggtcttttgtctctgaaggagggggtgaaacatggcatctcttgcctgtctcaagattcgtatgg

SEQ ID NO: 186 - DNA, 418 bp, Zea mays
acgatgttgctgccccagaatctgagaataaaggagacggcaagccagctgaatccgaatcagctccccttgcatttgttgatgaagcagcgccccatgtaatcgttggtgaagagaaaactgaggacagtcaggcagctgaatctgcggtgccttccctggcgtcttcagagggagagaagaaagagaacggtggtgccactgaaccagcagctccgcttgtggttgagacaaaggcagatgcacctcctccagctgatgcagacaagggcgcggagaattcaggccaggtgaacaccgccccccaagaaccgacggcagcgaactgcgacaaggggcagatccaacctggtgcctctgctgtcaggtgcacataagggagcttggtctccgatgagagaccgttgctttgtgtgtt

SEQ ID NO: 187 - DNA, 444 bp, Zea mays
cttcaagtttgccaatcttcagcccaatatcgaaggacaaagtaacacaagattagtttcaccaaatgcttaaaacaaaaaagtagtacatacatatatagataaaaatccaagacaatgaatggtctataaacatgtaatgaaagcaatagatttagtttctaatgtggccattggcagaaaattgaataaatagaactaacctgtccagttcggaagaacaactttgtgtagccaacttgatacatctcaggtaaaatgttgaactgatgaagaattgcaactgaaacactgagtggatcttgagatgcaacatcctcaagaagaagaaagccatacctgattaaattaagtattaacttttttataaaaaaaaagttggcagaaatttactgcagttgttcagagttaacttaccgccgagcaaacttctggtgagtcatt

SEQ ID NO: 188 - DNA, 747 bp, Zea mays
  misc_feature (461)..(463): n is a, c, g, or t
attttgaagcccaagtctttcagcaacagaaggatgaagtctcttttcacctcatagtcctagccaaagtcatcacaattgcaaggtaagacagctcaaactgaaagaagcatggttagaattaaggctcacttttctcagggatatgtagtaatctgctgtgcttgttaatgcaatcaatgcagcttcttggaatggtgcaggagctgaatctgttaattttatgtggatatttctaatagctgaggcgatgcttgctgctgcacaagcccaaccgagtctccaacctaatcagtccagacaggtagtttgtcaagtgtcatctcacaagtcttgctatttctatagctggttgtgtcactacaaactgtgcagcaaattctgagttgtgctcaccagtcacactgtaagtttttgacagtgaggatgtgatgatggtcctctcttgcatccctggaagnnnagccagtgagatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccccccnnnnnnnnnnnnnnnnnnnnnnnnnnnnnnnnnnnnnnnnnnnnnnnnnnnnnnnnnnnnnnnnnnnnnncccccc

SEQ ID NO: 189 - DNA, 579 bp, Zea mays
caggtcgctctagaggatccccctggaaggacatccatcgccacgccgacgtcaactgttctccatcaactggaagggaagtccagcaaaggtcccttttaatgttattgcagaccattccattgaaatgtataaatccagctttcttgtttgttttatagcatattttaccatatttatgtgaatggcggggcaggcccctacatgtatttctagcaatcaagcagtttcctaattttgcatctatgttttttttatcattttaactaattttattcccatttatatcttgcttatacaacatccagttttcttttctaggactagaatagattgatgtgaaggggaaagaatagaaggagaagcttaacttgtatattaagaggaaagatagaacagtgtacaacagagcatcaactgatttgtggaacaattttgtcttcattgtaatcttcttcgtcattgcataatctgtagatgttgtaagcgtagttaaactttattccatgcccaagttgttgcttatgtcgttaagttcttgagaaactcaactgattagattgaaattatgcggattgt

SEQ ID NO: 190 - DNA, 904 bp, Zea mays
ttttttcgtgttgctcactctatgcagcttattgtcaaccagtgaattgatcgcttgtctttatccttgggactttacatatatattcttgtttctcaacaataggctttggtagacaagacgaagctggacccaagtgaagttggcgacattgttgttggtactgtcttagctcctggttcccagagggcaatcgaatgcagaatggctgcactgtatgctggtttccctggtaacggcatttccaatgttgcattttcctcctgtggtgtaggacaaccatcatcccaaccttgatctatttgttggttcaaattggatgcagacaccgttcctcttaagactgtaaacaggcagtgctcttctggccttcaggcagttgcagatgttgccactgctattaaagcagggctctatgacattggtaggatttgttgctgtcttgttttggcttactttgttttgttttggattggttgatgaattgggttttattttctccattcaaggtattgctgctggtttggagtccatgacagtgaacaaagttagtcttgatggccaagcgaatcccaaagtaagcctaacatgttggtcaatatggcaaaactgcatatttagaaacgtgtgcaagcatctttgatctgattaaatttgtctcgagtctttctactgttttaggttgagctattttctcaagcacgtgattgtcttctcccaatgggcctcacatctgagaatgttgcacaccgttttggcataacacgactggagcaagatcaagctgctgtaagttgcctgactcccattctgtggtttgtttttttgtgtgtatatatatgagaccttcgtttgctgtctcacaatatgcttcctgctttaaagtcttataatctttattctg

SEQ ID NO: 191 - DNA, 490 bp, Zea mays
tactttccagaaatcgccacatagctgcagttctagcagtacagaatactgatagggcaacccgcgtggtcgcttggtgttgagcttccacaaaacatgagcttcttttctgcatagtcatcttagcctatcctcttgaatttcagtccggacaattgatacaacgacggctatgtcatccagtttgcccccactgtagccaaggtacccagccgagagagcagcatctgaaaatgggcttctcccagccccagacctccctacttcctgggccttcacagccaagtgttccgcaatctcctgcaagtagaacaaccattgcaattgagtcattttattacgcactggacatttttatttaccaagataattgattctgaccgctggttctagatctgcctgtaacgatttggagacaattccagctatttcatgctcgtatacattatcaaaaaggccatcggttgctgtcacaattgcatcgccgtcc

SEQ ID NO: 192 - DNA, 255 bp, Zea mays
tgcagcggcagcgagggggagcagacgaagagggcgaggagggaggagaagaggaggcaggcgaaagggaaggccgtggcgcctcagatcgacacgggcttgttcagcaacgtcccccctgagctgttcctgcagatcttcaagttcctctcctccgaggacctcatctcctgcgctctagtctgccgcttcatgaatgctgccgcgtccgacgagaccctctggcgtcgcctgtaagttggctggggaggctgc

SEQ ID NO: 193 - DNA, 888 bp, Zea mays
tgcatgcctgcaggcttcgtaggcctttcgcgccagacctggcgctggatgttgttgtaggccatcttgtcttgggatcatagagctgcacgaagagacatttttaacatctcttttgcactgctgctactccttccactctaaattgtaaaatatttttttatctttttattaacatattatgtttactagtatatgtttatatatcgtgtatataattaaaacattgtatctagcgtcttataatttacaatgaagagtataaatatatgtgattatattgatatttatacatatatctatgttgttgtttcgacgaattcataaaataactcgcttgttaggacttgtttagcaataatgtttctcaaaatcatagttttacaatattatactttacaattgttatgataataccgtagtattggcagttccaaaaaataccatggttttaagaaatattgtttgaatataacattgtaaactatggtataaacaaaagctagtcatgctttaaaactttaggttggactagagtttttaatactccaaaaatacgatggtatctataataccatggttttgaaaacatagctttcaggattgcaacaaaacactacgatattgctaaaaactatggtattgtctagaaactgtaaaaatacttcacttctaaacagtgcatatgtgtgtggagggtgtcttggacatataaaacacgttttccgtgaccaaacaagcagtgatgagtgaaacgtgctgacgatgacaagttgacaagtatctttgagaagctaagcaaactcaatttcaacgaaccattgctggggtaataatccttatgattagaaaaaaatttgagtaaattatttttgggaggttttttaa

SEQ ID NO: 194 - DNA, 765 bp, Zea mays
gagagctctgttatgcagtgaagaatggcacttgtgccgcaagaggaataacttggacgagccgtggatctacgtaatctgtagggaagcaggcatcaaatgctatgtgttggctccagatcctccaagccggtgctttcgaatcatgcatgtcattgaacccccatgctcagggaggaaaggtgttaccattgaagctttagacaaaaagctatttcttctgggtggctgcagttccgtgtatgatgctacggatgaagtatattgttatgacgcctcctcaaatcgctggagctcagctgctccaatgcctacagcaaggtacttaatcggcaaagtccattgacatctttatattcactctcgagatcgagacatacctgtcaatctataattatagataacatgacacatgtactccattaatctgtgcctgtatatgtagtgcctcatgttttccctagaacagcacatatatgtagcatgtcatattcgtccttaggcaacaccttatatactatatctattggaccagttccttatgctccgccgcctccctttcaggtgctattttgtgtctgcatcccttaaggagaagttgtacataactgatggatacggcttgacggacaagtcaccaaattcatgggacatctatgacccggccacggactcgtggtgcacccacaagaacccgttgctcacccctgacatagtgaaattcgtcgcattgggcgaggagctggtgaccattcaccgagcggc

SEQ ID NO: 195 - DNA, 388 bp, Zea mays
gcttctcccaagggagtgccgagagccatgcagggtgatggggtacgacgtgcccaagggaactaccgtgttcgtgaatgtctgggcgatcagcagggatcccaagcactgggaggacgctgcgacgtttagaccggaacgattcgaggccgcgggtacggttgattttaagggcacggactttgaatacacgccgtttggggcgggccggaggatgtgccccggcatggcgtttgcgcaggcaagcatggagctcgtgcttgcagcgctcctgtaccatttcgactggaagctgcctggtgggatgctgccgagtgaattggacatggcggaggagatgggcatcactgcccgaaggaagcgcgacctttacctgcatccagtcgtt

SEQ ID NO: 196 - DNA, 255 bp, Zea mays
  misc_feature (236)..(255): n is a, c, g, or t
ctggccgggatgtcagagaaaggaaaacccacaccggccaaaccacgactgcaaagagaaaggaaaggctctctcccagccccaaagaaaaaggcagcacaccaactcaccaagaaagtgtggagctgctcctgcctgcagcctcctgctcatcgaccctttcgccccacaagctcgattcctcgctcggccatggctgcacccgcggaggagacgcttgccgccgaggatacggnnnnnnnnnnnnnnnnnnnn

SEQ ID NO: 197 - DNA, 873 bp, Zea mays
cgtcgtcccgccaagggcagcagcgccgagatcatcccgttcctcaagacctgggtcaacctgcccatggcagtcggcttcatgctcctctacaccaagctcgccgatgtcctctccaaggaggcgctcttctacgccgtcatcttcccgttcatcgccttcttcggcgccttcgcctacgtgctctaccccatgcgcgacgccatccaccccactgcgctcgccgatcgcctcctcgcctcgctcgggcccagcttcctcggacccgttgccatcctccgcgtctggagcttctgcctcttctacgtcatggccgagctctggggcagtgtcgtcatatccgtccttttctgggggtttgcaaatcaggtacggctcacccaattctattaccgtgtctcctctgtcacagctaacaatttaatatttggttcacctgttgccagttctctactgaaatgctagtgattcatggctagacattttaaaattgcaagtagtaattgttgcttgctctgttgtggactctgaatgtccccttcccccaaaaaactttggaatggctggtggggacgtggggtatggttgtctttatgcgcagaaattaaaggagttctctccaacttcaaagaaagggcagttgatagttgtccgatgtggtgctgtttgtttattaatcttcaggtgcttttaatcactacagattactacagttgaagaggccaaagagttctacccgctgtttgggcttggggccaatgtggccctcatcttctctgggcgcacggtgaaatatttctcaaacatgaggaagaatttgggtcctggagtggatggttgggcaatttcgttgaaggccatgatgagcatagt

SEQ ID NO: 198 - DNA, 544 bp, Zea mays
tatgcagtggataagggcgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaatgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgctgctgtggcaaacccaggtgtcactgttgttgacatcgatggagatggta

SEQ ID NO: 199 - DNA, 463 bp, Zea mays
accactgcgccatggctacttaattgcaatggaggtttgcatttctccgagaacccggatgtcgtgattctgcctccgttcttgatccaggatcttccggtcttgttcccaccattactttccttaggaaactggaatggtttcaaccttaatttgtttgtgtgtgtgtgcacatgcagcgtgtgtttcatatggagatcggtgcactcatcttgtagcttaatttagcttgcatccccttcgctaacagagacagagagtaagatatcttagtcctctcgccatctctctctaaacatatacgtatcattttgtttccaacgactctccgaatgagtcgttcgattctcctgcaggatctgacgacgaaagcgatcatcaatcatatgccacatgctatatttcacaggttctatctggttaagtaaagatctggcgtgtggcatatgattgatgatcactt

SEQ ID NO: 200 - DNA, 255 bp, Zea mays
tattatggatgggtgagctcatctataatataaacgtgaactacatattcactgcaaatagaataaaagttatagaactcacctatttaaaagtttcctcataatatttatcttttgtgaatgtgaactactgaacttgggaatcaaataaaaaagcaacattcctttatatgtattcctttgaatttctgattgaatgacattcctataatcttttttctacgtttacatattactggcagtcacctctatgag

SEQ ID NO: 201 - DNA, 766 bp, Zea mays
aatgtgacattcgatttcttcactgcaaacattgttgtacgagcaacataactgacgtgtaaatgaaggggataagagcccagggctccaacttcccatgcccgagtatcaatgtggcaatgtggccctgatggcctgatcgcccagaaacatttcggtgcaaaatcgcagcctttaattttaactaatgaaaattgactccaagcagaataatgcagcaaccacgcaggcacgtagattgatcagaactggtcacgataaaatcagctaaaacaataatgattccgtaagctttatggtcaataaatagcatccatcaatgattacttgagaatttgaaacagatccaatatttcgggattgcatagtagcataacaaatacaattgatctgtattatccacgtccatgcaaccggtctaaattttggttgcaattttacctgccaaatcacaacacggcggctaactaaatgcatgtcagcatgagcctttctacagaaaatctaccttgctaattggaagtcactatcaaagtcagtcaagagacacaagaaatggacgtattgcctttcaatacatgagttttgactgctgaacgagtgtgatgtttccatatactctcccatctaccgtgcttcctcttcctcttcattgttttctgtttgggatgctagcttcacaatatcttcaacaaggatttttccttttgaaggagtgcataaagagaacctttcacaaaaatccaagaaaattggacagaggcaa

SEQ ID NO: 202 - DNA, 475 bp, Zea mays
  misc_feature (151)..(151): n is a, c, g, or t
atcttcggagtcgcttaggccgagcgaccgaagcaggaactcgacagcctttcccttgtcccagtcgatcaccgggcgaacctctaaaacctgcagcgtagcaaaagaattttttttttcgttgagtcttggagcaagtatcaagatcaanggcagtcgcagtagaacagtctaggctcctaccattcgcccgttggttactttgagacgagggaaggcctccaagacttgtttcacgagtcctgcgaccactttccagtcctgaaaaaaacaatagcataaagctctcagtctcaggtaaattcgctactgcttccctgtaataggcgcggtctgaatctgatgcatcctagtaaaaataaaaggatgtctgaagccgtaatttcatccatttctctaccgacaaaaaaaaggaaagataaacagatnnnnnnnatnnnnnntgaaagtttcaggggcacgcaatctcaccttc

SEQ ID NO: 203 - DNA, 851 bp, Zea mays
  misc_feature (758)..(758): n is a, c, g, or t
ctcggtacccctgcaaatgcacttctccatgcaattggattgtccaccttgccccgcccaagcaccgaatcctcaattcttggctgaatcaggaacttctcaatcttgcaggcagaatcagcatcaatgccagcagcatcaagcgaatcaaggaggaacatgcaagactgaaagcagtttaagaagtactgtgagaagggaagatcagcgcgatcagcgccatggtctatggctatcacaatcttaggagctagctgtttcaccaacccaaggatcgctggcagtggtggtgcacgagcagagcaaccagtcgggaggacaacaactacatcttcatcatcagcggcagaaatgaattctgcagggttgatcatatcagcactgatagcactgaactcaaagggaattccaaggtcagcagcaaactgtgcaatgttgtcacgcgcaagacacagctccagtggatggtgggaagctgtgttacagcgcttaagacactgaagcaacaaggtctcaccggcacgaaattggtgtgtgtctttatgcaccaccgaattcaatctctgatgactcgctagatgccaatgcataagtcatccggcatcaatgatctcgaccgccactcgtctgagccactcgcactgtctgagattgaagcgtgagtcaaggtcatcattgtgctgttgtcaggattctccatatatgaggacttcccccaccctcttttcttggggcatgccgggtgatttggtatgctntcccctttatgttcttctttcttcctctaattttctctggtggggatggttagttggtagcctacataaggggatcgggcctttttgcaacct

SEQ ID NO: 204 - DNA, 436 bp, Zea mays
gggacagaagtcagcaaaaagatgactgccggaatgagctccatgagtttcaagatgaaggagttgttccaggggcagactccagctgacaagattgttgagggtgccacctcagaggatctggacgggcctgactggaactcaaacttggagatttgcgacatgatcaacacagagaaggttgacagtgtggaactcatccgtgggatcaagaagaggatcatgctgaaggatgccagggtccagtacctctccttggtcctgcttgagaccattgtcaagaactgcgacaaggccttctcggaagttgcggccgagcgggttcttgatgagatggtcaggttgattgatgatcctcaaaccgtggttaataacaggaacaaggcccttatgctcattgaagcatggggggagtctggtgatgaactccgttatc

SEQ ID NO: 205 - DNA, 533 bp, Zea mays
tgcggctgtcggcggtagcggcaaatctacgggcaagcgaattgagatttgatagataagtagcaaccactgcctacctgtaacaaattttccagttcattctgtacttgacaacttggcgtcacattcagagctgcacgctcgcactcaacagtgcaaaagttacaacacaaaaccaaatgtttcagtttcagtaaccgtaactgcctccgacaagtgcgagacaatttccccaaaacgctttcactcgtatttatagatacaatccagacaaaagatctgttgtgtagttccaattctttgcagacatactgtaaggtttgctgtgctcagatctgctcgtggcaaacgcagtttagttacaatgtgctggcgacgcccaactcgagctcattcagcagagccacggctcccggagacgtcgcactcgtcaagcagcgctaggatgttgtgctcgggcgcgcttacggagggcagtatggcgcgccttggaactgcagatcgtgacctagcgctgctaccgtcaggctcca

SEQ ID NO: 206 - DNA, 642 bp, Zea mays
acgccatttgtgcgcacgcacgagcacgagcacgtcccctactagtgtgccggccatcactcggccaccagaacaccacagctcacggtctgctgcttgtcatgtcatgtcacgcggtgcagttgacagagccaaggagatgctcggcacgttcagcccccagctggagccctacacgtacgtgacgccggaggacaccaccccgtcgggaatgtttgcccgggggtcctactctgccaggaccaaggtacgtacgtacgtggatgcagaaccagtcctttttcggttcacacagaatccttcaggctcgatagttcacgaggtttggtttggcatcctgttctttgacagttcctcgacgacgaccgcaagtgctacctggagatcaactacaccttcgacatccgccgggagtggccgtcgacgagctgaccatccttccctccctccatcggtggcctgagcagaccagtcgtttcgttctgtgttcttccttttctatgttgcctcgtcctgtcctgtccctaggtttttgtttgttcaccgtgttgctgcgtgcgtctatatgtgcccttctccatgcgccagctgtctgctgcgttgacaccgttactagacagtgcaaatgtcaggtcgggtcta

SEQ ID NO: 207 - DNA, 417 bp, Zea mays
ccccttatgagatagcttcgacttctgacggatgcagcaccagttgcttcactttgtattatttacattttctactgggaaacaactgctggatacttcttgccacagaccgtctccgttggtgtcatcttggcccagaaagctccactgcttgcaccaacagtgacgagtcgtccgtcaggtcagagtggcagcggctgaaggagaactcgggcaccttgaatttgtgtgactcagtgtgttgtaacgcctcccaccgctctgcaagtccatcgccttccagcatccggaccacctctgacatcttgggcctgtgaccagggaggaactgagtacatagcagtgctacttgcaccatctcctccagctcaatgcggtcatatttgctccccaggcccttgtcgacaagtatgtcca

SEQ ID NO: 208 - DNA, 799 bp, Zea mays
  misc_feature (2)..(2): n is a, c, g, or t
gngccgtggtngcngaatcgcccgtgcagcagctcccggacagctttgccggcttcggccccacggacgccgagctcagagagttcgccgcagacatggaagcccttcttggccagggcctggacgacggcaacgagctggatagatcgttctacatggagagcctagggctaatggcgcagcaggcggangacgtcggccggntaaagatggagcccaacggcancgtaagctcccgctcccgcggcgagggcgctcccggntttggccctacggagatgaagccagaggcgtcgtcagctgctgctgaggtgctggacacagacttcaactgctgctcgcccacggtgatgatggataacgaggatgaggacagttncgaacagaaggcgtcggcgagcaacgcngccgccgcgccgcgggtacgcaattcttgaagaggagcctggatctcancctcaactacgaggccatcatcgagagctggggaagctcgccgtggaccgatggccaacggccaagcgtccagctcgacgatttctggccccacgcccacctcacggtacgtttatttccaaattcacgctgctcctcntcctcgancgatcgatngcgancgcatgnccgnngcgngcnctnnnnangcnncnnntgcnnnagacnnncnnagtncantcggtncatgcaaggttgcgtgcaaggctngattggcngggaagaaggaccgnttgggttggggaagnncgtnattcacgnttgggatggcggcggcaaaagccggttaaacgtaccggaaacg

SEQ ID NO: 209 - DNA, 531 bp, Zea mays
acaagctgcatatagaaagcgagggtatttatcatatgttggaggatttgaaacataattttacatcagctgcatatgaacctgaaccgttcgctatttctgaacaattgtaataaatttctttggaggaagattatcacttgttaataaaaaatccatcatatcataaatattaaatatatattcaaacttacgagcaggaaaaagcctgtatctgagaatgatttaattaagtagaagttagtaatcgtacacttcagctgcatttacatatttttttgctgggagtattcattttctagcaattagcacaagacaagggcactgaactacaatcagatcaggcggctcggcatcccccagcaattcctcgctctcgtgcttatctctactctacagctgtaagttctgtacatataacagtcatctagcctgatagtgtggtcgaaatgcttgattagatcccccagatagacggtctctcgatctagttttagcaacatcaacaactatcatccagccgtttatgat

SEQ ID NO: 210 - DNA, 255 bp, Zea mays
  misc_feature (123)..(147): n is a, c, g, or t
tgcaaaacaaggtttcagtaaactggctcttaaaccttggcccttcactgggcattgtccgcgttcgaggcattgggctaatacatacagaactaatgtctggaatcccagctatcttctccnnnnnnnnnnnnnnnnnnnnnnnnntccaccaggtactggtattcctatgtgtcaaatcagttcctgtgccacatgtgcaaccggaggaacgatttctggtgggtcgcattggtccaaaagagtataggctat

SEQ ID NO: 211 - DNA, 740 bp, Zea mays
  misc_feature (712)..(715): n is a, c, g, or t
aggtgctgtacctattacagggcatacttcaggcactaggagaaatgacaactatgttcgtcggcacagaagcccattgaattctggactgtggatttcaattgaagttattgttaactttagccagattgttgctgccattgttgttctttgtgtgtcaagaaaggaacacccacaagctccattacttgagtgggtcataggttacactgttggttgttttgcaacgttaccccatctctattggcgctatatacaccgtaatattgtaaatggtgagcatgagtcatcacatacacctcaagggtccgctcataacaactcaaatgaagcgactcatgctgcaagtgcatcagaacgccgcagaaatgctgcccgaaatgctgtgcttgctaatcctaggtactcatgccttctttttttccttaaaaaaatggatcggtatttacaatgaatatgtttgactgtcacttgttttgtttatttatggtgctgcaaaatggctatgcattaaagatgaatgccctgtgctatctgcaggatcaatgcgctgtttgaccacttcaagatggccctggattgtttctttgccgtctggtttgttgttggaaacgtgtggatatttggtggccgttcttctgctgctgatgcaccgaacttgtacaggtaaatgtgaactgctcttcaattgatgagatgtcactatgtcagnnnntttatcctgcnnctgactttctact

SEQ ID NO: 212 - DNA, 571 bp, Zea mays
cgcatgaggggagagatggaccaggtgctccttcctacgccattgccaacttgttcttgttcttcttcttcttccattatattggcggcttgactgaaacgttaatctgcttgcaggtccggagcgtgatgatggacaacatcgacagggtgctggagaggggtgaccgtctggaggtgctggtggacaagacggcaaccatgcaggggaacacggtgcgcttcaagcgccaagctcgccgcttcaggaacaccatgtggtggaggaacgtcaagctcacgtacgccatgttcttgttgttgtcttgcacattttagggtaacaatcgcctgtccctgtaactcttccactggtctttttgcagagctgcagtgatattcatcctcaccgtaattgtgtacgtcctgctcgcctacatgtgtcgcggcttcaccataccgtcatgctttccgtaatctccatccgtcccggtgccagtgcggaccaccagccctagctctagctgcgtcacaacttggttttccgggttctgtaacagcagcagcggtgctgcagttctctctcttgtacg

SEQ ID NO: 213 - DNA, 255 bp, Zea mays
  misc_feature (1)..(101): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaactccaacaatctgtaatcatattatacacatcttccacttacatgaacggatcacatcagctagtatacatgataagggtctcatgtccatgcacaaaatttgagtgtattttacaagccaccaccaccaccaattcccctttgatgacga

SEQ ID NO: 214 - DNA, 416 bp, Zea mays
aagcggatgcagtcggtggagttggcccgcgacgccagccagatgccgccgccaaggatcgggatcgagatcaggaaggttatgaagttgaccacccccagcatgcccgtgcccccgcgcagcatcttgctcttgctcttcctggctgctgtgctgtctcctggaccttctcttctcctcttcctcgacctgctgctgttgctctgctccgcttccttggctctgcgcctctctctctctctctctctctctctctctctctctaactgtcctgccttctctctgctgtgccgttgcctgctctgcttctttgggcgtcgggtgaggagacagagagaggggacggctggatacttgtcggcttttaacgaggtcgtcgaggagcacgctagcgcggctgttcggggagagcaacg

SEQ ID NO: 215 - DNA, 720 bp, Zea mays
attatttcatttcatccttgcatgtgcctttcctttccatccacccacttgatggctgtagtctcgatcgagttcgttcgttcgtacttcgtaccagacgatgaactcccacgcacatgatttagtactagttttaccatgcattgttcagtaaaagtatatgcttgcttgatcagtggttgtttcaatcagaagattaaaaaaacggaatataatattaaaaaaaaggggaagtggctagggaattcctcagtcctagctagctagctcaccggtgggaacgccatgcttggcttgggtgcaggtacatatatcttgggctattcgacagcgaagtagaggctgcaaggttgttcacctcggacgattctgccatttgttcatacacaccatgccttttgatttctctcttgcaatttctcttcttcttttatcatggcttttgattcccaaagggttgagtaccgactcgatattcgattctccctgccgtttcgtgaccccagggcgtacgacaaggccgcgatcaaatgcaacggcagagaggccgtgacgaacttcgagcccagcacgtacgacggggagctgctgctgactgctgaagctagcgctgaaggtaattaagtagctgctcgctgccatgtaatcttcagatgacgccgctgttaattattagctcatcagctttctgacgatcagcccttgtttttgtttctgctt

SEQ ID NO: 216 - DNA, 255 bp, Zea mays
acttggtctgaaggactgcagctagcagcatctatgggttttcagttccctcagtgtgaaagtgtacatctttcggaagtggttcctttagcaagcaaagatgcaatcaatcttatctcgtggctttgctcatgggacccacgaagaaggccaacagcagtggaggttttgcagcatcccttctttcagccatgcttctatgtcccaccttcacttcgcttcaaatctactggatatgcatcaacgccaccatca

SEQ ID NO: 217 - DNA, 868 bp, Zea mays
tctggagacaagtgacagatacctgcacccagcttcctcttctcgtctgtcgtcattttcctatcatatcaaaatttcagcaaccatgtgatccagattcagttgcacaagtagaataccatcatcacaacataaaaaaataacagcatatcgtcaaatcaaaaagcatagaatgagcattataagatatacctgcatctgtgaaccaccattttccggagctcctccagcttcctgttgatctgatttagctagaaaggaaaaagaagagaggaagtactaaatatccatgggaaatagaataggatacatgaaatctaatacttaatgaggcagtgaagatgagtccgacattttgtacgcattgcatccaaaaccacaccatcaaactgctattaacatcccaacaactaacaatgtatctgagcatcatacctcatcatcagtgtcttttgctaattttacgattgcaacttctcgagaagtatttgtggctgcaacaccctttgattcctcctccttttgtcttttttcctacaaggatacgttgagaaaaatataatgcgcaaataaaaggttctgacatgcttttattggctaggaagtagacactcatgtacatttcaaaaccaaaaaaacaaatagaggaacatgcatgactaaaaaacttcacctcactctcaactttaggaagaaaatggagccacttctcttcaaatttctcaagcaatgatttagccatcaagtggatgacattttgatcatcattgtacttcatcgcattggcaaaaattaatcgaacatcagaacatatttctctaacattggtgtaacaagtaccatccttcctttccattttcttttggat

SEQ ID NO: 218 - DNA, 767 bp, Zea mays
tacatgattattagttttcctgtttggttcagttttgcaactgctttacttctctacagagaaggctcacacgattctttatcagaaacattatccaaagagccaactgctgaatcaataagtgacgtcagtcttgttcagagggcagccttttacctttcttgggtgctatgcccttctaatgatgatcaatgcgagatgctatcaaacaatatattggaaatatcacattcttgggctaggaacaacaaaaagtgcctgagctaccaaattactatgaaccataggaggaaactgcaaatacccacagctgcgaagtcagagaaatttcatgtgtcaataaacactgtaagctcccttgttaaagaatttgatgatagctgtgtgcctgtgtaaaatgttgtagcacaactgcctttcctcaagtgcaagatggaaaactagcggatctccatccttcaaactttaacctaatattgcatcagtggattcctttaggagtattgcttatatcacctagttgtgctaacgagcagaactgtgacatccttctgtgctacacaagcactggggaagattctggtatcaaatgaagcacaaataagaacaaaagattattccagtaatgatggtttcttaggcagctgcattgatagctgagagatgggctttgattggagcatatctcatctttatctggcttgacgtagttgaggatatgtcctcattgatatttggttgcgaagatagatgtcatcgttctgtca

SEQ ID NO: 219 - DNA, 687 bp, Zea mays
tttatgcaagcaggaggtgtacttgagatcatggaaggcaattgagcagggacttcttgtgaaaggattagagatttttggaaggaacaggttgggtctttctcctaatatccaactctagatagctcatagtgatcagattggtgttccttgcacaaggaacttaggaaatttgtttcccttgatctccaaatttcttgaaattaacctttgtactgtaaatttgtttcccttgatctctttaccttgtgacaccacataaacgtatccgagatctttgtactgtaattgtaaaaaaacaatttaatcttggtttacctaatcttaatagttacaatgaacccatggtttgtctttcacacagttgtttaattgctcggaaccttcttggtggaatgaagacgtgcaaagatgtttttcaatatatgaattatattgaaaacaacagtgcctctggagctcttagtggtgttgattctcttgtcaaaggatatattaaggttggatgcactgtgacccttatttgttgatacttacattcaagccggtaacttaatttggtgttgtccgtgttgatgtcccagggtactgagttgcgcacaagatcaagatattttagaaggcgaggtaaagtccgtcgtttgaagtacacctggaaatctgcaggttacaatttcaaaaggatta

SEQ ID NO: 220 - DNA, 431 bp, Zea mays
aaatgtctgccttcatgagcttccagcaaagcgtgcaagctgcaggggtgccaggcagccagcccaaccagacgacggagctggaccttttccaagacctgcagttgcctcaggtggagtactacctcccatccgcaaggccatttattcaccctccataatttctaattttgacaacgtattaacatgaatgaaaatgtctaattttgagcaggccggggcgcagagcacaaacccattctccgatgagagcgttcagggatacccacagtacatgaacgggcccaacggcgacaacgcacagccaggccaggacttccaacagcagtctctgaagctaccggcggcgagcccgtacgacaatttcctcagggctgctggctgttaggagtccagttacatggacgacaaccacctcacctcaagatagt

SEQ ID NO: 221 - DNA, 654 bp, Zea mays
atcttcgcatgaagcttaacgagtgttctgctacggagataaagctagcgcaggccattgaagatgagatacatttaactattactgctgtgctttcagcggatgacaatagaaagacagcttttcaacttgctttccgcgaggatcagcaaattatcagggtaaccgagtctttctttaattcaaccaacgcaaatgcttgttaaggagaaaatttgtcatgcagtaactacttgcttttcttaggggggggagctaactatttgctttctgtatcaggataagtggatacatatttcccgtgctctgatggatgagagaggaccatggtctgctaacccttttccaaatgatgttgtgactcactggaaacttgacaagacagaggatagatggaggcggcggttcaagttcaaacgtaattacaaatttgatgaacgattatgtcagccttcacagtcaaggaatgaaagtacagcaactttagctgaccagctgtacattagtgccaaaattactcagaaaatgaagcaattccttgttaaaggagtgcggggaatcacagaggatagtagttacgaacaatttgaagacactaatgacgcgagtgaatcttcccagagtaatccttcagagagccaaaaccttaataat

SEQ ID NO: 222 - DNA, 255 bp, Zea mays
tgcatgcctgcagattaatgaagctgaaaaagaaaacaaactgtatactgtagtcgtttatcttgaaaaagttcacacctgcaaaataacaggtgaattagataagctgcttcaaacaaagtacaattttgttaagatggtcattgttggataccctacctatgatggatccgaaaatgacaaatacaatcagatacactgtagaaaaaataaggaagtacatataatatataatccaataagggccatccccaa

SEQ ID NO: 223 - DNA, 738 bp, Zea mays
ttgcatgcctgcagcttgcacgctttctttgcagttttgctttttagtaggctgttgtgtatatgaccatgttaagagaagtgccatagacagttttgaagtttttagggtttaatagggtaagatattcaccactcagcggtatcagtgaccctgtggcctcttgaattgatgtgcaaataagtgccaaaagttgtaatagtatacaacgctcatggttacaaagtgattatagtcttttttcatgttttcccaagcgtggttaaacataatcatttttatctatcatgtatttaatatcgtgggcacatgatcaaaggggcataactcacatgcatttgttagctagctttcagagtacaaattatataggttattaagtatttagatactcgtcttatttcctgtcgctactcgtgcacctgctatctttaaatatttcgttctcacaaactgactggaaattgtttgtctctgacagtgctagatggggctgcgtaatcatatactccctctgtcccaaattaaaaattgttttatctttttaatagattcatacaatagttaatgtatgtgttctatatatgtatctagattcattttcatccatataagtatagatataaaaatatagagctaaaacgtctactattttaaagggaaatttggatccataccattaaaagatcaccactgtggatccataccattactatctcacttacatgtgggtccaca

SEQ ID NO: 224 - DNA, 471 bp, Zea mays
agatatacaactttacatgctacataggcacggtgcagcagggaatcggaagcagggcaccccaatttgacccaatgccaagaacggaaacttgccagcacgggcttcacaatatatgctaaaacgactcacgttttaattcttaggatcacatgtgatctttacacgaaataatccgggtacaacgaaggaaggtggtagcagtgggagagggcttggttcgggaggccacagtggaaattctctgtatatcaatctagtgttgggccgagagggacttcctataaaagcatgagaaagagaagaaatgtgagttcagctcaggattatgcataacaggatagaatagcaacggaacatataatacattgctcagcacgtctttctcctgtcaagtttgaatggaaggttcagccatttcttccttgggaaccgctcttttgatttcttttcacttgagttatcgctgta

SEQ ID NO: 225 - DNA, 255 bp, Zea mays
cccaatcctgcgtttgattcaggaaaatcaagctgagtttgtccgcttggtgaacgaaactccggagggtggtcctggagggtaagaaaaattaaataattatgttaataagtcacgagtacataaatattacattgtgctgtggtgaaccagaaacatattaggtcaactggcagctgctatgccgcaatcggtgacagttaccccagaggaacgagaggccatccagcgggtacagctcttttataacttatt

SEQ ID NO: 226 - DNA, 444 bp, Zea mays
cgtgatagtacatgctacctacctcccaacaactcacgtgtgtgagaactgttttggttgacactgcaaccagccacacgacaacgacgtcacaactccggactccccatcagtgggaaggtagggtaggggagtaggggacacaaacttgtggaaatcaggaaccccacgacgcaatcaagacgccacggttgctttgacgctggaaataaactcctccatgctccggtagctggtgccaccctctgcaactgccttggccgcttcctcggcggtcctcgtcgtccttgccctgaccgcttcgtcggacatgacctcttttactttcttcgcgatctccaccccggtcaccagccagtcctccccgtcccagctccagtgctccatccacaccccaaccccaccgctcaccgctaccttggcgttcaaagaaacatagatgat

SEQ ID NO: 227 - DNA, 255 bp, Zea mays
  misc_feature (172)..(209): n is a, c, g, or t
gccggccgggttccgatttttatgaaaaaggcataaaaaaatcggacgaacctgtcccgcgaggagtttagcgaagggcttttaaaaagagaaaaagggagaaaataaaggcgcagacgaaaaaactgcggagaggtagcggtggatcgaaaccctaggcgcggtggggctnnnnnnnnnnnnnnnnnnnnnnnnnnnnnnnnnnnnnngagtcgtacatcgggagcttgataagcctcatgtcgaagagcgaga

SEQ ID NO: 228 - DNA, 771 bp, Zea mays
  misc_feature (735)..(736): n is a, c, g, or t
tgctcgaaaacaccttgctccctggggctctgtacacatgaaagaattgcagcgtgtcacagcaacattggtttttagaagcaatactaattgtactccatacaaggttagcaacattttcactgttctcagttatgatatgccactgtgtcacggaattgggcaactgctggaggtagaattgtagaaataccatgtttctgtatggcatctttggtttgtagctgacagtcaatttgagctgagactagaaaagtctttttgagaatatgacgactcaaccaaattagattttgtttcctggcttgagcatgaattttgaaacttacatgtattgaacgttcatcggttatttgggatcgactcctggtggagtactgctgctgcactacaaatgttttgcatacctgaacctgatactgttttctatttgttagatattgtttgagcaagaccgctgggactatcttgttgacattttcaagcaagatttttgcaagttatatggcatgacactagagccgctgctgaacatatatctgcaagcaggcctgacagctctcaagaccccgtatcctcgcatgtgtttcacttacttttgtctatggtcttttattgctaaaatacagtttgctagtattatttttgttatggtatcctgtattgtccttgaccatttatttaaaggttctgctctgagggtagctgtcccaaagaagaccccttgtctctggnnggcttcaggaaactnnnngagcctctgcccttctc

SEQ ID NO: 229 - DNA, 385 bp, Zea mays
agctggcgtctgtaagggaagtacttaaaaagtcagaaggagaactcgagatcacctcgaagcagttagttttggtttcagaagcacatagtgaccttaataaagaattgctggatgcatataagcagttagagtccacgcaaagtgagctagtgaaagagcgtaaaatcaatgctactcttaacatggagcttgaggctttggtgaaacaatcagtgatagagtccgaagcaagaaaaactcttcaagtcgacttggatgaggccactagatcactaaatgaggtgaataagagtacactctgtctgtctaagcagctggaaacaactaattccaagatttctgctattaaagaggagaaagatatgctgtcaatgttccttga

SEQ ID NO: 230 - DNA, 518 bp, Zea mays
cttgcccttgctctttgctcagctggcgggtaattcagtaattgataactgtgcatcgttctttagcaaatattttaacaatgaaatgggacggttgatgtgtaaccattttgatttgtccttcagtttttttgttttatcaagtttccctggtcggcggaagcatgcaatagacaagattaacaaggctgctgatgaactctctcgaaaagttgatgaagctatccaaaaggatatctcacagagtgctaataacctaatccgcttcgttgaggttatcagcaaaccttaccaggaagcatgtcagcgaaaaatagattggttacagggcgttcagggtgagctatctgctgtcgagcgtaagctccaaactttgaaagttgaaatccaaaatcttaatggatcataaggtttcatttcaacttgtttatttgcaattattttttggatactaatagtttttgttggtttatactgcgttgtgatgctattgagttgacgtagtgcctttcctgtat

SEQ ID NO: 231 - DNA, 402 bp, Zea mays
  misc_feature (389)..(394): n is a, c, g, or t
ttgaatttctttgattccatttcttgacagcataagcatccacctgtggatgtgtcctccaggagaaaacatgtgaacccgccgagcattcatttcaaacttcaaaatagggccatcatggagggagagaaacgttatgacgaagtaaacaaatctgtcgctgtcaccgtcttcattaccattgcctaaagcaaaatcatcttctctgaacatcaattcagtcattgaagcccatgtgtacctccatttccttgacaagagacatgttcttacagcttcttttattggcaaacagcagagaattttatctttaataacatcaggtagattactgatgatgtcaacatttgtaacagatttatgtcttttcttttgagaacctccacgannnnnngggtatac

SEQ ID NO: 232 - DNA, 255 bp, Zea mays
ccacgcgtccggagcgcgagctcttaaacgggcgcacgccgcgtgcgcgcccgacccgcttgccccgcgctcggacgcgtcccgatgcgcctccgccggccggaaaacccctactagtcctagggctcgcctcgccgccctgatgcgggggccgcccggggtacctcaccctgccgatcttttccgtggtcgcggcgatcgggtacgtctactacaccacggtgttcgtggccgtgccgcggtggctgggcctgt

SEQ ID NO: 233 - DNA, 461 bp, Zea mays
acgcggcttgttcttattgaatccatcaaggacgcaggaaagataggatcttcggctccgattctcggagcaacagcgcgaccgcagcgggttttggtgcttcctttctttagggttgtgtccttgcagaagttgctctgtggaatggggacctgtcggaggagagttctgggggtatggtttttcttctggttcttgttgacacttgagcagtgcacatccctgaatcgtgaaggtatctatcgtgttgtggtttgaatcgcttgcgtggatggttgttgcccgacctttgtttttgactctgtttagggttgtttctttgctgaactgcaggtgctgctctgctgagatttaaggcggcgatcgaggcagacccatatggtgctttgttggactggaatcaagagagtttgagcccctgtacttggtttggtgtggaatgctccgatgatggactagtc

SEQ ID NO: 234 - DNA, 458 bp, Zea mays
  misc_feature (89)..(90): n is a, c, g, or t
gcagattcagagtggctcctggataagtcctgggatatcctgaacaattcatcatttttatgtagtagagatagagttaagatcattannggcatggatgaagcttattatggatggatagctcttaaccacaacatggatatgcttggcacctcatcatctaaaatgacatatggttcacttgatttagggggatcatcattacaggttacatttgagaatgataaatcagtgcaggatgagacaagcattcgtctgagtattggttcagttgaacatcacctcagtgcttattctcttactggttatgggttaaatgatgcatttgataaatctgtagcacatctagtgaaaagactgggaggagttgctagcaatggcaaggttcaagtcaaacatccctgtctacagactggattcaaggaagattatatttgttcttactgccatccattgaa

SEQ ID NO: 235 - DNA, 255 bp, Zea mays
  misc_feature (249)..(255): n is a, c, g, or t
ggggtgttgaaggacgctcggttcctcaggtgcggcggcgaccttgtctaggattttacccgtggaacgtggcgaccacttccgacgggccactcgccggcgctcgcccgccctgctcttcaccccttctcccgggaccgggacaagcgatctcccgtccccgtagtccgcaggagagagatttttttctcggttgcaatgaagagttatccgtgggcgggggcccgccctcttgccgtcgtgcagcgnnnnnnn

SEQ ID NO: 236 - DNA, 255 bp, Zea mays
  misc_feature (120)..(123): n is a, c, g, or t
ctgaaccctggggttcctgaccagctctgccatcgcccactcgactgagatgactgttgtgtccattccagcagtgatcatatcctgaacatgatgaacaaaccatggaagattagtacnnnngaagtgatgcagaaggtgtccggtcatgaaggcnnnnaggttgatgccgagctgcgctcctgggcacacatgccggccaannncgaacnnnnnnnnnttgaagtcgctgcccttgatgtcgatgttctcctc

SEQ ID NO: 237 - DNA, 478 bp, Zea mays
  misc_feature (433)..(434): n is a, c, g, or t
tctttggatctctcttccacatcattcaccaaagcattttcttcgctgttgatgtcttgcagtggcataatttcagccgctggagcaacaacagacggtgtctcatcaactttttcttcatttgtgattgtgactagatctgaagcaacttccacgtcaggctggatatctggtaccagaatatccatctttgcattctccgatagattaacattctgaggttcttgattctcaacatgaattggagcttgctttgcacttcttagtggtctctcacattgtacctcttgacggtcaggaatctcagttgttgggctaggcacctttgaggtttcaaccatggaactagtaaccttcctaaggctacgtttaaccttctcaagttctgttaactggccatcgggaactgactcagcagggtggcctacttaannccacgctcggagcagggttctccattgctttgctagcagctacc

SEQ ID NO: 238 - DNA, 788 bp, Zea mays
ctgatatttatgttctcgggtaatttttctctcagataaacctttttttctctctttcgggtgggttatacttaaacatgccgccgacttctaaagggaaaaaatcaataacgtaaaccttttatatatattttttgcaagaaaattgtccacgtacatatttatttttgaagaaattatgccttctcaattgaaattcatttaccaaacagatgcagctggacaagtatattaaaggtggcagttaagagagctgttactcatatactgtaggcccacatgctatttggttatgaaatttgacactacctttttatttttgaatcaacattaaaaagatgcagaactgcttcaattactaatattccttacaaattaaaaaaaagctccatttaccatgaaaactgccgctacctttcagatttcaagagattatcccactggaagtcgggtatatgattggaactgaagacactcgtccggtcgcagtatgggagcatatcatccacgaaactttgaacaagaagtgtccggataaatcgaagtccaaacgccacagtgactctcccccgccagcaaggtttaatccattagactatgttcttgccgtggagaatgagttgcataacgaatccaacagtgatagcgatggggagcttcatcggccgcacaatgcagaatcatccgattcagctcgacgccgtctccagccattggatttagcctgtggcgttcacaattataatggggataaaaggaaaaggctgcagtatgtacgggtaataagc

SEQ ID NO: 239 - DNA, 571 bp, Zea mays
tgcgtcgtcttcgatttctcggacccgaacaagagctcagtggagaaggatatcaagaggcaggcattgctggatcttatagagtttgtcgagtcgaccaacgctcgcttctcggaggcagccatcgcggcctgctctaggatgtgtgccatcaacctgttccgggcgttccctccgagttgcagatctggctcatcaggtggtggtgagggcgacgaggaagagccaatgttcgaccccgcgtggtgccacctgcagcttgtttatgagctgctgctgaaatttatcggatcctcgtccttggacgcgaaggtagggaagaagcactttgatcactcattcatcgtgaagctccttaatcttcttgactccgaggatccaagagagagggattgcttgaaaactattctgcacagggtatatgggaagttcatggtacaccgtcctttcatccgcaaagcagtgagcaacatattctaccagtttgtctttgagactgatcggcacaatgggatcgcggagctgctggaggtctttggcagtgtcattagtgggtttgccttgcctctga

SEQ ID NO: 240 - DNA, 255 bp, Zea mays
  misc_feature (129)..(140): n is a, c, g, or t
aaattcggcacgaggcaaggacaaggacggaggaggagccattaccgctgggccttggacaaactaacggccggccctcgagggcccacgcgtccgcaacactcacgtttctccagacacagagggcannnnnnnnnnnntcaaggatcaaggatgatgggcagtgggatgggcgcgtacggcggcgcgctgcgaccggtggagagcgcggccggggagacgatgctgctgtggggcctggggcagcccgagtcg

SEQ ID NO: 241 - DNA, 771 bp, Zea mays
aaataattcaatacaaaatcaaattcaagccatcattcagatagcctttgttgatacaactgatgacccatatatttgatccttatgttctgtttacagttaagactatgagtgcaacctagagaattggtgtcgctaagacaggttacaaggtgtaaggcaaaaaaaatacagcaaggatcaaaataatacagaatacagtatagattatagaagtcaacagacgtcgacagcgtttggttgctaggttggtccaaatattaccaataaagtcaaggaacacctaagcaaagactgttataataagataatgctacagcaagctattaagtatctaacaaatgattaaagtttaaaatcatacaaacttacatcaacagcatcggtacccttatccatcagatcaatttttgtgagcacaccaaaggtcctttcacctgaatcaacaaccgtgcacttctataaaacatgtacatgacttaagataaacaatgtacaaaaattacacgaatgcagtaaaagtatttctttcataaactaacttgagaacaatggcaggaattatctgtgtaatgtaaattaactttcaacagagatggacaaatcgacatttggtgtatgaaaatcacaagactgtcagtccataccaattttcatcatatcaaacaaactcaaaattatatatcattatgaagcttcctggcatgccaaaagttaaagcatatgtgacaataatcattaaagaaatgtatagtcttatattgtatacgg

SEQ ID NO: 242 - DNA, 904 bp, Zea mays
  misc_feature (1)..(30): n is a, c, g, or t
nnnnnnnnnnnnnnnnnnnnnnnnnnnnnncgcagatgcaccagtgtccttccagatcccgccgcgcgtctccttccgcgacacgctctcagtctcacgccacagctcgtcgcgttcgtcaacggtcgacccatcgcctatggaaggaaggcgcgaaccggaggcggacggcggaaaggccgggggcctcccgccgtcggagccgccgcacctgcagggccagccgccgcaggagtacgggtacggcacgttccagggccctcgacatcctcccgtcgggttcccgcagcctgccccgcctcccgggctcggcagcggcggctatcacaaccagcagcagccgtacgcgcctgcggagacgtactacgcccaggggtaccaggccgtaccaggctatggtcctgttgctgaaggaagacctgtgagaatgaggcgtctcccatgttgtggcctcggcttaggctggtgtctgttcataaccgggtttttcttggctgccattccatggttcattggagctttcgttcttatatgtgtgcgggtacatgaccaacgagagaaaccaggatacgttgcttgcacaattgctgccgttgttgctgccgttgccatcctcttgggggtaactaaaggaacgcacgtgtggtgaattgcctccagagaaggcaatcaatgtgtatttgcgttgtatatctgtgtgtgctaaaaagtgtggagcaacatccatgtgaatgtgatcttatgtccatatatattctactttgcaatgaaatttgaaatgtagtaatgtaccatctgcttcttgtaagcagcactgcatcatatataataagataccttttatttaaaannnnnnnnnnnnnnnnnnnnnnnnntgtgtattatnnnnnnnnnnnnnnnnnnn

SEQ ID NO: 243 - DNA, 530 bp, Zea mays
tcttgccgagaagaacacgcgggacctgctggctcaaagagagcaagcagagaaacatgtaagacatttacacacaaagaacattctctctgtacctgcatttattcatagcattggtgtttatgtatttcagaggctgtctgaattcctggatcctgagatcaagagatggtcaaatggaaaggaaggaaatctgagggccctgctatccacattgcaatatgtaagcagctaaatttcatgcatccttaaacccgtaacatagaacatggaaaaggttatcacgtgtcgctctgtttcccctggcagattcttggtgccgatagtggctggcaaccggtgccactaacagatctcattacagcggttgctgtgaagaaagcttacaggaaggcaaccctatgtgtccatccagataaactacagcagaggggcgctacgatcagacagaaatacatatgtgagaaggtttttgatcttcttaaggtatgtgctctgtccacattgaatgctctggttatttggaagtt

SEQ ID NO: 244 - DNA, 909 bp, Zea mays
acgccctggcagagaacacacgatgcaagtagagtcaattaggagcggtgttcttggtggtggtcatttcttcttttgggagatagaaatatttcggctcctcagaaacttaatatgttcctcccccacttgctcacctctttgtttgttatggagacgttccttccaaccctggcattcatgtcgatgatgcagttgctgcaacgcatcaatagaagggcttattaaaccatcgtgttatgacacacaatacttttcttttttgttccttcttccatcaaggttcaagccaagcgctcaccttatcttcgtgttctcccctacgccaatgggcaccttgccctctgccagtagccttgagatctcgtcttcggtctcatacaaatccgcacccatcatcatggtattctgcaaagcaccgttcgcgtcagtgtctgcgggcgctcaatacagatacaagactgagcagtcatcgacgatgaacaagcaagcgaaccttgagctcgcatccagagtttaggcgtgagcgaacaccaacaatagagtgctcgatggcacactcacgcaagaagcagccgtgtgaaatgatcgcgtctttaatctgcacacgttgaatccaaaggtcaaataggcacaagctcatactacagtttacaggtcacaggcaccattcattccatgcataggaagcgcgtaccctgcacttatccgacttcgttggtggcaagtaccgaggggaagtgaagaatggtgttttcggatcgtaaaactcgaactttggaggctgcgacggggcatacgagatgaaaactgtattagtctggatggaaatctatcatttcaagatattttcagtctaaatgcactggacagttaagagcacattagacctggaaagagaaaacagaa

SEQ ID NO: 245 - DNA, 745 bp, Zea mays
tcctcgcagcagaaacgctttcctaaagttcacaagaaaaacaccatctcaactttgctgcattgatgcaacggaatgaacttgacagaatagaaatgggaaggggctctgtataacatcactaacctctgtaactttctgggattgccgattataagggttatcgtcattcataaacatttccatctgagcagcacttaatccataaaggaattgaggaacatgcttgtaattatccatcactgccagaggacgtaatacttaaaaatggtgcaaagatttgattgacaagatacagaagcaggtgtaaaatgttgtggccgctacacaagtcatgcatgtgtaaaaggtgcagagcttgataaactttttatcgaagcattcgatgggtgtttaggaagacaaaagaggtcctctgatgcatagtcaacaaataaaaaaaataaaaaacagaaggtgcaaatatcttttttttatttatttttcttatgaaccagctagtaaacttggtcatgtgccaaaaagttaaactgccatctagaatatttagacctgtgccaaagatttgcaagctcattaaattggaaatcatcaaatcaaggcgaacattgtccctgaagtctaacactttgcggtggataaatgaacgtgtagatactagtgtttcgctgaactgggtgcccacggttgacagagcactcacgtccatccttgaggttctcctcgcggaactgcttggggatgt

SEQ ID NO: 246 - DNA, 255 bp, Zea mays
  misc_feature (54)..(97): n is a, c, g, or t
cagcaacaccgccagggagagataaatcaagaccaaacgtacccaaacccttcnnnnnnnnnnnnnnnnnnnnnnnnnnnnnnnnnnnnnnnnnnnnctctgcagcctccaccttggtacagagatgaacatatagctttggataagcctgagcttcctcctggtgtatcaaagatgactgaatatgatggaccanagtgtttnataactcccggaaatcatgactggtttgatgggcttcatacttttatgaga

SEQ ID NO: 247 - DNA, 825 bp, Zea mays
ctagcttgcatgcctgcagtgaatcttgcaaagtagtggcacgtgttttagtttttagtgcggaaggaaaaaaggtaaaataaaaaagaagaggaagcaaaggtggggttggattggtgaactgggcagggcatggcaggacaggagggagggcacggggcaccagccaagccaaccatagcaacttctctaccctaggcgtctctgtcagcccactccgatttgggccgccaccactagtgatggcccatcaccgccgccgcctcctccactctccctccatccatttaccaccccaccccgccgccgccctcctcttctctccccctgagcctgactggaaccttcccgagagaggatacaaaggcacccggatcgaggaaaccaatccattccattccattccacacaaggctaggcacaagcgaaccaaacccatggcgtccgacggcagcggcgtggtgacggtgtacggcagcaacggcgcggagctgctggagccgtccaagcagcccaagtcggcgactttctccgtcaaggtggggctggcgcagatgctccggggcggggtcatcatggacgtggtgaccccggagcaggcgcgcctcgcggaggaggccggcgcgtgcgccgtgatggcgctggagcgggtccccgccgacatccgcgcgcagggcggcgtggcgcgcatgtcggacccgggcctcatccgcgacatcaagcgcgccgtcaccatcccggtcatggccaaggcccgcatcgggcacttcgtggaggcccagatcctggaggccggggcgtcgactacgtggacgagagcgaggt

SEQ ID NO: 248 - DNA, 847 bp, Zea mays
ctactgtctgcaagctcgaattaaaatacaaaataggatccgaccttgaacattatttccagaaaatgacctccaaagtccaaagtgcctctttgtacaatagaatctagcataagccttattttaagtgcccaacttgtttaaaggaaaaaatcttaactgacaaagttttaaaaggttttcagagcattaacactactctaaagtttacactggacaaggaaaccattgtaaatgtacattgtggtggcctaaatatgtacctccagtagaccctattgattactctacaatttcttgttttcacacaaaaaaaacctctgcagtttctgaaagtttgtcaaaggagactttggtaaaagtaagatattgaattattgatgcaacaaatgaaacaacaacactctagtcagttggatggaccattgggctcaattgcattgtgcaacatattcgcaactaacgaagttaaatggcacaacatgacctgttaattatgctgacagtttacttaatgtgtttgcagctgggtattaataactcaatattttccaatatctcatttacatctttttcatacactcattgtagctaggaactttcgaatgatacttgggacaagtgacattttgacaaatgctgcaacgacatattaccttgggaggaagatttgtcatttcgaagtatccaccacgttgtccgcactctccccagtaccctttcgacacagtgtggaaagaaacaacctgaagctcactgcttattggcggacccatgtcgaacataacctggaaaaaaattgagaaaggcaacataagcaatgatttttggaatgccaaacaatctt

SEQ ID NO: 249 - DNA, 255 bp, Zea mays
cggacgacgacggccagcagcaacagctgcagtcacccccgcaccaccaccacgagatagaggacgacggcgatgaagacggcgacgacgcccgcagcgacgcgcctgcccgccacggcggataccactcggtaagcctgcccgtctccttccccgccgcgattccgattcacctcggcagctttgctgttccccgtcgaacctgcctggggctgggcagggcttaggttttcggattcgttcgtgggactcggg

SEQ ID NO: 250 - DNA, 255 bp, Zea mays
gagaaggatcgttcatgctctttgtgtagttccttttactactattatgcgcacctgaagcttgcatgacgaaaaattgtatacataagtgcacacttcttttgggaaatttattcacaatgtgttctcactgacgtcatgactattaggttgtgagctgattagcaaaacatgggaaagtttccatgatgtgaagctcagtgaccggagtctcatttggtatgtttttttctcataaggaagcgtccatttcat

SEQ ID NO: 251 - DNA, 627 bp, Zea mays
agtgagtttttccagcctattgcgtccagccaatgcaatttcccagagtagtcccatcactttcattttttgaacttgcagatcgccgaagatgacacccaagatttcgactcaaagtaccgggcacttgtcgcaatcggttctcttgtatccttcctgaacatcccgttattgcagggcttcatgcgatgcacttctctttccttgcatcatttcttctaaattttccatgccttggcatgtgttgcccttaacctaatttactatagatgctcaagggccttgtgaagtcccttgccctcgaccttgatgttaagagcgttgcaagcagcgccaagtcttcaatggaccagaagattgcggaagtaggaggtgacatcgaatcgctaacaagatgactacaataggtttgcgtacacttggtcgttgcggacattgctgctccttcgcttgaggcactaccgtagtaccgtgcataacttaaagccggtctgtgttgcagaccaattcagacgccctccacgtctctaccaaaaaaatcgcaatgggttatgattattttttcacctttgtaaccatcatggaagtgtggattttgggtggtagcgcttagattttgtaaattga

SEQ ID NO: 252 - DNA, 713 bp, Zea mays
taagatatactatctgatatctctcatacataccatacattaatgctcatatgtataaagaacaaacacaacgccatcgtccatgtctatatactgtacaattgtaagaatgataacacatttccgtgacctttacgcgatgttaacactgcttgcttcagaaccatgaactgaagcagaatgtattgtataacattgttactgatttaatgtttgttcgagcaggagacggagaagattgcgctcgcagaatcaacgggcctagaccagaagcagatcaacaactggttcatcaaccagaggaaacggcactggaagccatcggaggacatgcctttcgtgatgatggaaggcttccacccgcagaacgccgctgctctgtacatggacgggccgttcatggccgacggcatgtatcgcctcggttcatgaactacacacttccatggacgactgtgtggggcctatatatccaatcgtcggaccgtgggagtatatttcagtgtaccaggaccaggatgcgttgcagtgcaaggacattgttgtggaatttttgttgctgattgccaaggggtgggtcggtcgatgtagcgtctatgcatggcttgtagttctcctgtaatgttgttgttaatttccgcttggcggcttgtaatcttaggtgtatctctggaacttgaatcctgggtgattaatccggcaagcagctgcct

SEQ ID NO: 253 - DNA, 412 bp, Zea mays
tgggacgcggtcggtggcgcccccatgcccgagcgcatcatgaagcccttctcggtcagctgcgtcgacagtaggatcgtcgtcgttgggcgaggcctccacgtcgcgattgggcatgtgcgggaggagcccgctggcggcggtcctggtagcaggggagcgccaggctactccgtttgctggcaggacgtggacgctcctaaggagttcagcgacctgacaccttcgagcagtcagattctgcatgcttagctgctcgttgagccagccagccatctgcccactggtaatccgcacaaggacttcaacctggttgggttggttactacgagcggtcagcggtcaactattgcagctgcctgcttccagcgcttttgcctttgatgagatgcgcaatcgagtaggatctttt

SEQ ID NO: 254 - DNA, 255 bp, Zea mays
actaagatatcaaatcattcaaaaaatagaatataagataccagatcattcatgaattttgttacattatgcaacttcatggcactcagcatgatgaggactggcattatcctgaagaacataaacccaagcatgagggctagtccttgtaattttccaactagctattttctatagtactgaaccatgccatttctagagaatctttactgaattgcaatcagaaagtctcactacaataacagaaactaaggg

SEQ ID NO: 255 - DNA, 255 bp, Zea mays
gcctggctctggcttctgccatcccactcagcagtgccaaggcccagcgccgaccgagccttcccctcgcctgatctgcgcgtccctgtcctcggctcctgagcactcgcgtactcgctcgtcgcatctgcccacactaccactggccacaccgaaaccctagctgcgcacccaaatcgatcgatccagggacggcagcggcacgacgcgccaaaataggttcagaactagcgtcgcgacaaaatcggttcacga

SEQ ID NO: 256 - DNA, 468 bp, Zea mays
tcctcctgctcgtttgttacaccggtacgccttcgccatgatatataaatatacatgcaaaaaaaacattgtgtagattacacatgtttgtttgcgttttgcagggacgcaaggcgggccggtgactatggtgtcggcgaggaagtgcgagtcgcagagcttccgcttcaagggaccttgctcgagggacgccaactgcgcaaacgtctgcctgaccgaaggtttcaccggcggcgtgtgcaagggcctacgccaccgctgcttctgcaccagggactgctagtactgcctcgtctcgcctcgcctcggctcgtgtcctgcgtgtgcatatatagccgcagcggatcagtaaaacatgcatgcatgcgttaccgaatatttggcagtaccagcaagttaatggattaaaatattaaataatatgtcatggaacaaagcctgttttgaacgtggaactgctgagtgcta

SEQ ID NO: 257 - DNA, 388 bp, Zea mays
ggtgattcgagttccctaggagtctcggccatgattgtgacctgggggaagtccacggaacacgacgggtgtgttcgcgaagaaatcaagggcagagcatctgggcggcgtggcggattcctcatcggcgtgcgctgctcgtcgctgggccgccattcctcgtcggtggagcatgacacttcacaatccgaggtattatggggggaatgtgtttggggtgttcaccacatcccgcaacaaggtttagatcgagttgcggggcactaggggtcgggaatctctcgcaccggcggatgctccgctgttaggggaggttgcgccgccgtcgggcacaccataggatgacgacggtcgcttgaccgtagatctccatacggacaactttcat

SEQ ID NO: 258 - DNA, 374 bp, Zea mays
cgtgagcatggagagcacgattccgacaagaatgcttcccccgacgaccttcctccttccatcagatgaagctgacggcacacttccctgccctgggtcatggccacagggaggcagagggatgccgcagaggccagaattgttggcataccgggactgcggaaacgtggtgagctgcccagtcgaaggaatcggaccagacaggttgttgctggatacgtccaagtcagcaaggaaagtgagaccaccgaggcccggggggatgccaccggtgagatggttgtttgagaggtcaagcgcgccgactagcttgagccctgagaactcatacggtatcgtgccgttgaggtcgttgtgccccaggttgaggactt

SEQ ID NO: 259 - DNA, 255 bp, Zea mays
gggattttacctattgcccccctgtttttccctttgtgaagcttaagctacaccggctagcttccctcagacacagaacatcatcagctcactctcttctccatcctcttctcggttatcccaccaactccccaaccccaccccaaagatcaggtttcgatccgcgaacccactcccaatcctccaaagatcaccgctctcagacccctcctccccggcaatggccgccggtggccccgcgctgctgcgtgccct

SEQ ID NO: 260 - DNA, 555 bp, Zea mays
aatatagttttcaaagaaagctgttgtttcccgtactgatatggcagttgtggtttgtgttgatcatgttcagatttccaaattccattggctgttgaccctcccaaagctggaagtagggaaaaaaggagttcaagggcatctattgctttaggaattggaattcctattgtagcaatattcactgctctgattctcggcatttattgtattaagaagcagagaaagagttcgatgcatgaaggtaccatcgttatttttctcatttcaattattttgttatttaataaactggtaactctctttcagtttttggttaaatccacatgctatagcaaacctactgagctgcactaggaattgcaaaataccttggatcccaaataaacagagattcataaattttttttgaaagaaatggcaggagctctgcctaatcattaaaataagagagttcagaaaacacaatgtacaggtccgaggaccgagacaagacagactagcaaacacagcgtgaggaacggtcccaacagacccgcacaaaacagctaaacc

SEQ ID NO: 261 - DNA, 781 bp, Zea mays
agggatgctgaaagtaattccatacctagaaacactttcatacactcatttgcctcaacaccagcatctgcacaacaataggaaatagaaagtggcttaaccagttcatctgtatgtcggaaataaacaaaaacggatgaaggcacagaaacgtgcaagggacatccacacagatgaacacacagcgcaattaacttgtatgattgtatctcatagagcaatccttatgggactgatagaaccccaaaagcatgatatctcttcaggtttgcagaaaccaaaacaggtctacagtttgaattatgtcaagtgcactccacaaagaaagcaaaatgaaggaaaatgtaagatgaatagaatgaggttcaccatttccttatcacactttaacctgtgtgactatccaaatcctctaaagacagtgcattaggggcttaaccaaacaaaaggttagatccaatgctccacaagcagacaagcaataaattacagacaatgcacagcctcgatccagaaaacagggcaaagcctaacaatttagcaacttctttccacctcctttttactaatagagaataaactccccacgcttcaatgtcgcaaccaccaaattaagcaaggcagacacatctaaatgagaagcagcagcctctcggctacgctccacaggcatatccggcggaagccgccaggtcaaaataaacataaaaatgaacacacgccggaacccaacaagacagcaggcgcgtcacaacacgaatgggatctgcaggcatgcaag

SEQ ID NO: 262 - DNA, 442 bp, Zea mays
gtgcacactggcgccagatgggtatgtgtgcagcccttgatagcctgagcttttggcaaaactaatggtctgtgacagactaaccgatctttggttttttgctcttggacagttcaatgacatgatcaacgtgccattcacgtcgaagccgttcgtcgctgggttggtagcctatatcctggacaacaccctccaggtaaaggagagcgcggtgcggaaggacaggggcaaccactggtgggagaagttcaggagcttcaagaaagacgcgaggagccaagagttctactcgctgccgttcaatctgaacaagttcttcccgtcggtctgatctcaaatggcgccgccgctgaatcaattctggaagcaacccttgttcatatgggccttaatgaggaacataattctgctggtctggccagtggaagcttctgtgtctcca

SEQ ID NO: 263 - DNA, 255 bp, Zea mays
tggatggtactttgtttgacaacgctaacatcatgctgtccagaagggaattctattccacagctgatgttctgcaaatattccagcaggtcagttatttgaggagaccagaaggctattatatcatcgaacaagtttcttggtgatgtcattactatttttgacatttagttaatggttgctttcatgcaacaacgctatttgctgagatcaatgtttagttttttttactgtgaaataggttatcatcaagtc

SEQ ID NO: 264 - DNA, 255 bp, Zea mays
  misc_feature (2)..(10): n is a, c, g, or t
cnnnnnnnnnctgagatcctagggattcaggatgaaagatattggcccttgatgtctccattaccatcatacggctatggaagagagcacaagggacctcgatatggaagccttatacacggccaagacctgaagcatgtgactataacaggtcataatggtaccataaatggacaaggtcaaagttggtgggtaaaatttcggagaaagctccttaatcacacaaggggtcctctggtgcagctcatgcggtca

SEQ ID NO: 265 - DNA, 602 bp, Zea mays
  misc_feature (542)..(553): n is a, c, g, or t
gtagatgcggtcgaatatcttcattctggcaagagtgttggcaaggtataagttgtctgtctatctgtgacagcatgagaatctgtcccaatcttcgcaacttacaggttttcatcctgaactttttcaggtcgttgtgtgtattgatccatcatacggtcagatcgttgcaaaattataatccatggacatgatgcgcattgtcttccatggtcacgtaggttactgtcttatagttacacatatacctcaagaccgtattttcccagcgggtgacaaatgaataagggaacatgttggtgtatagtttagtttcatttttccaaagtgtaagatgcacaactatactgaagggcaagcatgggcatggaaatcgattaagtttctaatcccgtgcagtaggatatgcattcgagtggcctcgttccacgttgtatgtatacaccatgctcttgtcatgtacgcatccaacaacactcccttgtccttggctctatctatgcatccacctttcactcttcttttcatccttgtaatagaannnnnnnnnnnngtagcagacaaagaaatggaaannnnnnnnnnnnnnnnttaacttgttc

SEQ ID NO: 266 - DNA, 889 bp, Zea mays
  misc_feature (877)..(877): n is a, c, g, or t
tgagaagcctgagctgacttctccggaggatcttggtgttcaagctgcatcaatgctactagaagaggtggctcaaggaggtgttgttgactctacacatcaggtctgggcttttatgcaagcattctatgtatttctgaattttgcctggtgttaagctcgatgatagctgtatatattgttatgggctcagtgaagcttagacaggagtacaggacagtgaagcattttgttcttttggactgtttgtgtctactacagacaagctgcaagttctacatggcatgtggataactagatatgatttttttcaacgatctactttctcttaaaaaatgttgtcctctcttggactgacagtgctgtacatttatgtttgcccatcttaaattgcttgttgtctatcctgatgatattccatgcataagttacagatgcttttgtttagtgattttatcttaaatgcttgcagggcctcctatttattctgtgtgctttatgcccaccggatgtatcaaaggttcgtgtgggacagttgactccatatggtatagagacacttcgaaacatcagagattttcttgacgtcaagttcatcatcaagcctgatcccaattcaaacaccgttactttaaaatgtgttggcgcgggagtgaagaatcttgctcggaagatttcataagcaacatgaccattaatctgatcaaggattcatgccacatcaagagactactatgagaccctatttgtaactgcaaacaacagtgtaggagtttcccttagctgttactgctgaaattctattcgaggtccatgatgtcttagacatcaccgatgtacatcagtattagaatggattgttctgatcatgatgccntgtnnnncgaca

SEQ ID NO: 267 - DNA, 697 bp, Zea mays
taatgtaactggggtgaataatcagcaaagcagtgagcgcacatgtgtgtagtagtggtgaactggtggggcgtggagcactaattagactgcttgagtttggatcatacatatccaaatccagtaacaacacgtagtggcctttcttctgtccattcagtcatctttagcaaatcttccatagtatgtatgttctcaaaaatgccttccttaggtacctggtcgcacaagttgactgcttcagatataaacttcaatttgaaataacaagactagtacctatgaatcctcctggataccaacataccgcaagggacaaacgacaatgtccaaactcaagagcatcatgaacaaccactaaatcctcattgccctgacacaaaaaatttaagcacatcggtcagataaaggcttcttcaaagcagaatagagcagacccagtgtcagaggctctggggaagggataaaccgatgcaagcctttccccacgaatgtggagaggctgcttcgaacctgcgacctggtgacttagtgagaaagctagcaccactgcaccaggcctaaaatttcacataaaagaatattgcggattcgattggaagagattaatgttttataatatataggtccagaacccaatgtgcgcacaaaccaggaaggcattagtctaagcacaagataaaccagatataacaaa

SEQ ID NO: 268 - DNA, 391 bp, Zea mays
agggagcggcgtgccgcacaggcacttgttgtgcgcgtagcactctgcctcgtgcatggacatgaaccttcccgccgggagctcgccgcagagctcgttatagctgacgtcgaagtgatccagcctcacgtccatcagcgacttggccactctgcccctgaccctgttgtggctcaggtccaggaacctgaggtggtgcgggaacctgacctccgtcaggtcgaactcgagctggttccacgacaggtcgatcttgaccgccggcttcgtgatgccgaacaggaacgacgaggggtcaccggtgagctggttgtgcgagaggtcgatggtgtcgacgtcgccgtcaccgtactctccgggtatctcccccgtgagccggttgttggacagg

SEQ ID NO: 269 - DNA, 892 bp, Zea mays
atgattgtacggcaatcactcagagaataagggctatgcttcagtcagctgatgaacaggtcaggagcttgaagaagcagagtacatttcttagccagttggctgcaaagacaattccaaacagcattcactgcttgtctatgcgtttaacaattgattactatatcctgccccttgaggaacggaaattcccaaggagtgaaaatttagaaaacccaaatctctatcactatgcacttttctctgataatgttttggcagcctctgttgttgtgaactcaaccataatgaatgctaaggaaccagagaaacatgttttccatcttgtgactgacaagttaaactttggagccatgaacatgtggtttctgctgaacccacctggcaaggccaccatccatgttgagaatgtggacgaatttaagtggttgaactcatcatactgtcctgtgttgcggcaacttgagtctgctgccatgaaagagtactatttcaaagctgatcgtcccaccactctctcagcaggttcttcgaacctaaagtatcggaaccccaagtacctctctatgctgaaccacttgaggttctatctcccagaggtctatccaaagttggataagatactcttcctcgacgatgacatagttgtgcagaaagatttaacagggctgtgggatgttgatcttaatggaaaggtcaatggtgctgtggaaacctgtggggagagttttcaccgttttgacaaatatcttaatttttcaaatccacacattgctcggaactttgatccaaatgcatgtggctgggcttatgggatgaacatctttgatctgagggagtggaagaagaaagaattacctgggatctaccacaaatggcaaaatatgaatga

SEQ ID NO: 270 - DNA, 768 bp, Zea mays
tgttgcttcttgatcagttacagttggttttgccaattcagtttttcccacgtcaccagtacgtacatattggaatgaatcagagtcttgtggagcctcagcagttggctgtgcttgagttgatgcaggtcgcggtgtctgctgatcatgtaaagatcttgtgacaacctcatcaccaatagcatgatctgttgctgtctttttatcaacagccacatcattagttgacaaactggcatcataaacatcttcgtggattgtggcaggatcttgtgaagcttttgcagcctctaatctttgaccctcatatgtgctgagaagctcaccagcttcatgactatcaggagttcttctcaaggtaccttttgaagcaggatgagcagatgtaattggtgcttgcatttgttttggtttccccaccctttcatcaatgttggactgctcagtaaatttatcactgatagtggcaacatcagttctggcagcttctgggagtgatggtgcagcacgtctaacatcagatgcttctacaacaattggtacaggtggttcgaccctttgctcaccagattgtttggccatctgattgacactctgtgaacttggagctgcttcttgattcatgggtcttgacttttgatcattgttaagagatatggctgcctcatcatcagcagaatccataattacaccatgtaatgaagcagccatgcgttgccattgatcaatggggaatgatttgctaggcaaaatcccccttcagggaac

SEQ ID NO: 271 - DNA, 593 bp, Zea mays
tcttgcatgcctgcagttacaggagtactccaaaagcacagaacgagcacagcataaattatgacctatttgatgaacaagattagtattaataaggtatagcacatggcaaaacaagaaaaccagacatagaaggtagttcacataggagcaccttagtaaccaacaaaaattttgtgtatttgtcgtataaggattgcttccctttaaggtcatgttcctgcactgtaaagcaggcaaaggaaaaaaaagttatgaaggttaaaatgcatcatgggagcatcacctcattttgtacaaataacaaagaagctaaattttgctcccgaacaaacaacagcaatgctacggaatgcaactacctagcatttccaaaaatacagggccttccggtactgtattacaacttacaagcgatgagggcatgaggccaagacgattagacgaatagctacagaacaagacaatgagagttcagcactcactttttgccagttccttctccttggcagcagccaggcgcttgagtttagcagcttgtgcaaatgtggacggcctacagcagacatacaggcaaagaagcgaggagtatt

SEQ ID NO: 272 - DNA, 424 bp, Zea mays
  misc_feature (415)..(419): n is a, c, g, or t
tgtctgtgaggaaagtgatgcttttgtggctaacaacattgggaacatggccaaaattctggctgggcaaaggtaatgcatcttttgtaactgttcttatgcttaactgctgttaacttgttaactcaaccccccctctcctgatcatctatctctgcaggagatattttggacacaagagaacaatccggccaaatgctaaacagctctatcccttattcatgaagaggggaaatatgtcatgggatgcatttgcctcacaggtgcgaacaatccagaaaggatatatgggagagcccatggaaatcacaccaggaagaggcgaatttcatgcaaaccctgccgcctgtatatgtgaaaagaccagcagaaactcagcaatagtcaagtccatttctggaaataacctggaacnnnnnactga

SEQ ID NO: 273 - DNA, 255 bp, Zea mays
cctgggcggccacccagacgtcgttgcgcttggcgttggcggcggcgtcgttgatccgcttggccactggcgagtggtcagctgccgagctattgcgcctgctctcgtcgtcggcgccgccattggtcgcctcgacgtggagaccgacccggcgagtcactgccgcggccatctcgaacggtgagccacgcctagttctagttcgtgttgtgttggttggtgcatgcggcctgccgaccgagggttatatagggc

SEQ ID NO: 274 - DNA, 554 bp, Zea mays
ggttcgagtcctccgaagtcaagtccaccccgttgcagccctacagcgaggtgttcggcctccagcggttccgcgagtgcgagctcatccacggccgctgggccatgctcgccaccctcggcgccctctccgtcgagtggctcaccggcgtcacctggcaggacgccggcaaggtacgtgaatagctagctattaagctatgcatgcatgaatccatggagttcgttctcgatctgctctcagacgatctaaccaacctgcgaattaatgtgacggccggcgtacacgtacgtgcaggtggagctggtggacgggtcctcctacctgggccagccgctgccgttctccatctcgacgctcatctggatcgaggtgctcgtgatcggctacatcgagttccagcgcaacgccgagctcgacccggagaagaggctgtaccccggcggctcctacttcgacccgctcggcctggcggccgaccctgagaagaaggagcggctgcagctggcggagatcaagcacgcgcggctcgccatggtcgccttcctcggctt

SEQ ID NO: 275 - DNA, 374 bp, Zea mays
tggtgttcttctgcttcctggtggagcagcggcggacggtgcggagcagcaagccggcggcggggatctgctcgcgctgcggcgggtgcgccagcgtggcggacatggagaccgccaccagggtctgctacctgctcaccgtgcaccgccgcacctggcgcgccatcatctgcaccttctgcggcgccatgctcaagtcctaccgccactacaggctctactgactctcggccgatcgatggagttgttcgagtcggtgggtggtggtggaacgcgcgccttcattcttcgcgggcctcgcggcttgctgtgaggtgtagtgatgagctacttacggtactagctagtgatctgttcttcctgatgagtcctca

SEQ ID NO: 276 - DNA, 491 bp, Zea mays
tgatgaacagaaccagtagtaagcactgaacagtaagatgtcccacattgccaataagtctgccagcttgaaatctgaatgaagaggccagtgctatatatgtttgaaacagatatagatctgacaccagcatgatggtaccatgaatttgacaaagtcactaaattgctgagcttagatgaccaatctcgtctatgcaccccatcgtgctaatgccctattacaaacataagaaacgccaatattctcaaggacaaacctttggttgctgaggattaaagcataatgaactatttaacaaacataactcgtatcagtaccgatcctactaacccagtcaatcgcaatcagattggatctgatactgatcgccttttgatgaaagtgttacgactccacactatctagttatctgcattgttcatggaaattccacacagcaatggccatctgcatccataccagcaaaccagaacttttatctgccctca

SEQ ID NO: 277 - DNA, 357 bp, Zea mays
gctgcggttctttgtgaagggggaggtggcattcgtgatggcgcacatgaggcggtggaagcgcacggacaagaagttcttcggcagggcgagaaagctgttcgacgttgaggtggttcacgaggatccaccgctcgagggctggcggcacgggccggtggtttaccggttcaccgccaagaagcagcacggcaagaagtgaagaaactaccttaaggtaaaggctgcttgcttgcctatctgcatgtgttcatgttgcagcgctgctgctgctactgtttgtcctcgtttgttgagtgctagattccaatgttggaaacagaaagtagttccttggaacaatgtgcaactctggac

SEQ ID NO: 278 - DNA, 345 bp, Zea mays
cgcttcgtgttcgacggcaagcggctgaaagggaagcagacgccggaggagctcgggatgaagaaccgggacaagatcgactttttcgttgatctgagcgcgacggacgacggcggcgacgtggaggagccggccgtgtacttgacggtgaaggtggtggtgctggacatgaaagggcgtacgatggagcgcactttgcagagtacccacaagctgcaggtcgttatggatgcctactacgacagcgtgccggacgtctcacgcggcgtgggcaagttcctatacgacggcgggcagttgcagggctggcagacgctcgcacagctgaagatggatgacaaggac

SEQ ID NO: 279 - DNA, 155 bp, Zea mays
  misc_feature (40)..(40): n is a, c, g, or t
tgcgacatagttgccttcaacaagatctaggcgtgacttncgatcagtccttcccctggggtgctggcgatggtggtggctgggcaatgcaccagcctaatttatccatagctatatgtctgctttgtgtggccagtgatattgccgtgagtatt

SEQ ID NO: 280 - DNA, 373 bp, Zea mays
gatcgaggccagcgactgtgttccaaaactaccttggcgatggcagtcggttcgtgccatcgaggaggtcacagatttatgggccaacactgggtgcagatggtcgcttgtggatggaaggcttcaacattgtccagtgtggaaattacgcacaatcagtacctacggccataccagttgctcaggtgcctcagctactttaccagtcagaggatgaactagcggcgaggcgaacacgcagtcacatcacaatctcatcgtcagttgggtcccctataaacccgctccagcgctctccgatcagagcgactccattgcgagtcggttcatacccccggtcgacagaggtgtccgtctgtactgctgtgaatat

SEQ ID NO: 281 - DNA, 456 bp, Zea mays
gccggtcggtttgtatgcaactacgtgtactaccactctcttcggttcgcggagcaacacggcatcaaatccctgttcgtgcatgttcccctcttcttgacgatcgacgaggaggtccagatgcacttcgtcgcttccctccttgaagtcatcgctttctcctccaactaggaacagcagttggaaggtgcgtatgctcagctttccttcctgtccagttcattgctgtaggacagaatagcagagtgagcgtaacactgtcctgtccgggttggtttcctgtttgcagatcagcgtctggattcctatctcaaggatggatgtaaagctggaatgctgcatgtaggctgaataaggtggtgtggtcattggcgatagatgttaatcttgctccacaggttgtgtttgggaatcgggagggaaaaaaagagaggtcattggcgatagatgatgatc

SEQ ID NO: 282 - DNA, 407 bp, Zea mays
  misc_feature (398)..(400): n is a, c, g, or t
gggtcttggcggccatgcatggggcaacgatcgagatcgacccggggcacgacggcggccgacggcggcgaggagggagacgatcgatccaagaggaggtgtgtccgtgcgtacgtgtgtgccgtgtgcacacggcagcgagcagctagggcatatgcatcatttcagaagagaactagctactccgatcaagatgagcgacgaaggattgtacgtatattgctgagaggagatcgaagagacggtgatgagttgcattggcggaaaggtcgatgcatgcgtggatgatcagccaatcgtacgtagtacgtgtgtaaaaggtcggccggagcgaataatggtcgtcgtcgtgcatgtctcgatcgagacacctctctcgctcgctcttcctctcctannncttaaaa

SEQ ID NO: 283 - DNA, 255 bp, Zea mays
  misc_feature (1)..(1): n is a, c, g, or t
natccatccatcccacctcctgctccaattctcccctccgcccaccaacgcagcagcagccagcagccccgccacttcccccctcgccgcttgcgatggcggtcacgcgcgtnnnnnnnnnnnnnnnnnnnnnggcggccccgctctcgtcccgccgccaccgggttgccctcccctccgcctgccgccttctccccgcctcggccacctgctgcaacagccgcgcgccgatgtcgctgnnnnnnnnnnnnnnnn

SEQ ID NO: 284 - DNA, 562 bp, Zea mays
gcccccagatgtcgtctcctcagataatgttgagtacgatttctctgatgtttttggttctaccccggtccaaacaccaacaaacctttgtggttttggtccggagagtcctgcacctactgttgagtccaatgaagaattttacagtgatcctgttgtcatcatcaaccgatcccattctcttgtcggtccaacatcacttgtcagccgctccttgcgacttagcaagcttagtctaggaaaaaacgagggatcatcggaacttgtcaagtgcctctcagaagaaaaggaaggggaccaagggcagctcagtgatgaagatctgggtaatgcgatgacagaggatgagggcattgggcttgatgacttagaaatcttgaagcttgttggccaaggggcattcggtaaagtatttcaggtgagaaagaaaaacacctcagagatatatgcaatgaaagttatgaggaaggacaggatactagaaaaaaaccattctgaatatatgaaggccgagagagagatattgacaaaagtcgaccaccctttcatagtgcagctgagg

SEQ ID NO: 285 - DNA, 512 bp, Zea mays
tgcggtcaataaggttgcagttcctaaatttgagccaaagaaaggggtgaatattgtgacagatgagaaggctactaacctttctagcgcctcagttgatgatgttgctgttattaatgatcttttggccaaactggaagaatatgccaagggcctgccaccagggttccaaatgaaacctatccaatttgagaaggtaagtattccaaatgaaactcaatttgagaaggttctttgagatggttccccaacatgcacttttcttttattaggattttgtttgttcgagggtcagatggatatgaaaaggccaggtataacatgcgctgctttctttccccaggatgatgatactaattttcacatggatttaatagctgggcttgcaaacatgcgcgcaaggaattacagcattcctgaggttgacaagttgaaggctaagttcatcgccggaaggatcatcccagccatcgcaacttcaacagccatggccacaggtctagtgtgcctgg

SEQ ID NO: 286 - DNA, 840 bp, Zea mays
aggaatgtggcaaactacatgggtcaaatggccatggctatgggaaaacttggaacgcttgagaatttccttcgtcaggtattaaccctcttttccaaatgcatctttgtgacatagataaagggttctgtgaatgattgtactccctcccaaaaagaatgctcatcttgcatttcagggatagcttttttaagtttggccagacagaaaaaagtactaaactactaatatttatttatgaatgcgtagtaagtatcgttaagttaatcatgaaatatatattgataataaactcatttgtaccttcaattgttggtactattctttataaacccaatagcacttaaaaagatttgattatttaatatgagggcattagacttgaatatatatagcgatatacatgatagagaccgtgtatagaacaataactttatatatgagatgaaataaaacaccgacatatgcagagacatctagcaatgacacttatgtatgaaactgaactgaatacgacaaggacaacctgcggtgcctaaatcctgctacttctctttgttgtatgtatcaaggctgacaacctgcggctgcagactctacaacagatgcaaaggatcctgaccactagacagtcagctcgggcgctacttgtgataagtgattactcttcacgtctccgtgccctaagctctctctggcttgctcggcccaaggaatagcaagaacatgctatttgactgcaatacttttcacaatttggttttcaatggtcaggagatttgacaaggctgcgacagacttgattcggacatatctgcacatactatgacccaaatgaag

SEQ ID NO: 287 - DNA, 862 bp, Zea mays
atcttgcatgcctgcagacatgtaagttcttgcagtaatcctgactcttaaacgtcatttccacgagtgaggtgatgctaatctgacttctcttatcgtcgtgttcgcagtgagcttgttgttctcagtaggtccggtttcttcagtgatgtggggagaggaaagatcaggctctcatcgtttcggtatgtccaggaacgccactcactctgaggatcaaacagcttgaacaatgtcggccagaaggctcaacccgtctatgaaatgaatctacgatgtggtgttgcaggcagaaacagagtgccggtgctgtgcgacttttcgagctcgaggtccgacgtgtgcgagctgagaggcgacgtccgcgtcctccccaacgccacgatcgtcctccaccatccgctggcgaggcgccagtcttggaggatgaagccgcacggcaggaagaacgaccggcacgcgctcgcccgcgtcactgaggtgacggtgactgtggcgacggcgtcggcgtcgccgcaccacacctccggcgcccctgccgctgctgctccccgatgcacggccaaccacaccgccccggcggtcgtcttctccgtcggaggctacgcggggaacatgttccacgacctgacggacgtcctcgtcccgctgttcatcaccgcgcgccggttcggcggcgacgtgcacctcctcgtcggcgacgcccagccgtggtggctggacaagttccggccgctgctcggcgggctctcccggcacgcggtggtggacatgggcaggggggagcagcaaccggggccgtgctcttgtaccccgcacgtgatcttagggctcgaggtccaccaaggagatgaagcgtggc

SEQ ID NO: 288 - DNA, 499 bp, Zea mays
ccatcctggcatccttttaagatggttgaggttaacggagaaaataaggtacttgctcagttctgtccagctatggtgttatctttaatgcttatagaacctctattagtatttgtttggaaaaaaaaatcatatccaccctcattcgttgcaggaaattctggatgacgatgacacaaaactgaagttcctgtggatcgagtacggcgatgatgtgtgcaatgcggtgaagaccgcactgatggagattaacgagtataaccccagcgggaggtatgttgtgccggagctctggaacttcaggaaaggccggaaggcgacggtgaaggaggtgctcaagtacctgttcagtcagatggacacgacgacgaagcgcaggaggggctgaggaggtgcctgatgccgagtagtgccttctatgttgttgcatcagtcgtgtgctgctcgatccccaagtggcagtctggcataggagatgcccctcttgtgtagtgaaatc

SEQ ID NO: 289 - DNA, 470 bp, Zea mays
aggattgcctgaacggaagcactttctggaacaccctcaggccacattggcaccacaacataaacagtgaaccgctccccagcctcaatcttgctgacaatcttcaatgagagctccttcggaatcaagtgaagacaatcgatatcttctggcttgatgccttccggcttccagtcatatgagcttccaaggaagtactgattctcaatgtagatgaagttcttcgccctccgtatggcatttatgtatgcatcctggatactccggtcgatgatttgatcctttccactcacaagcccagctcttgcagcttcatcaggagtctcggggaagccaaaagcagcaccgccgtcaatggatctaaagagctgaacattccaggtgtctctgtcctccgggaacatcacaggagaagggggaataataatgtcagaaagatccctgagacatacaaggaggtccttaccaca

SEQ ID NO: 290 - DNA, 255 bp, Zea mays
  misc_feature (1)..(18): n is a, c, g, or t
nnnnnnnnnnnnnnnnnngaccatttcgctgtggctcggatgacaaaaaccctcgctgcgccgcgcgctcccctctaccgcctccgacaccgcagagccgcagccaagcaagctgcgcccctggacttggcctcaccggtgaagcggagcttgaggataggcggtgctcgggagataccaagcggtcggccgcaggcgccacgtcggcggagatgacggacggtcaccacttcaacaacatcctcctcggagggc

SEQ ID NO: 291 - DNA, 736 bp, Zea mays
cttgcatgcctgcaggggcaggtaggcagctgcgcttacccgactgtctgcggcgcgtacggcatctgcgtcagcgggcagtgcacgtgcccatccgcgacgtacttcaggcaggtcgacgaccgccggaccgacctcggctgcgtgcccgtggccccgatctcatgtgcctcgacgcaggaccaccggctcctcgctctgagcaacgtttcttacttcaactacgtggataccaaggccgcgctgcctcggatgatcgacgaagagagctgcaagaaggcatgcttgcagaactgctcctgcaaagccgcgttcttccagtacggcggcaacgacacctcccggggctcctgttacctgccgacgcaggtcttctcgatgcaggtgaaccagtggcaagaaactcactacagctcttctgcgtacctcaaggtgcaggtcacaaggtctcctcctcctcctcctgtccctggtccctcgaattcgaatgggacggccatacccgcagggaaaggaaggagaaggactcgtgaagctgccatcgttggctccgcacttgcgggggccattgctttactagccgtgatcgtcgttacctcggttgttctccggaggcgatatcggcgtagagatgacgaggacgacgaggatggccttggagaagtgccaggtatgatgacgaccaggttcacgttcgagcagctgagagcagcgacagagcagttcagcaagatgt

SEQ ID NO: 292 - DNA, 510 bp, Zea mays
tgccttgcgtggtggctgctgcatactggtggaaccctcagcgccaaagcgtgaagagaagatatcgttgccaaacattgacctctggaatcctggttccgctctcatgccacccatgccaggggatccaaagaactccttaaatatatcatccgcactcctgtgattaaaatggggagcggtggaatgagccccgccagggaagaaggggaatgcgccagaacctcctgctcctgggggcggctgccccttgagcccttcttctcctagctgatcatagattgcacgtttcttgggatcgctaaggacctgataggcggcacacaggattggtcaagtcaacagatgcaagaactgaactagtagcagtaaaaaaagcgaatcatggggtgaaatgcataattcggcggtgcagctaatcagaaactcgaatcgataagctaaaatatggatcaaaagagagacatctgagaaagttaaatcctttgcagtattggcgggcgtggtcca

SEQ ID NO: 293 - DNA, 255 bp, Zea mays
atgagatgatgactgcgtccagaacaataaaaaataaggaaagatgcaattccaatcttcctatccttacagatgtcaattctgggttgatagaccttaaggtcacatgaaaaatagtaaatctgttaatagattgttttagtcgtggtgagttattggttttaatagagaagcatgatagtgatgatatagtaattactgtgcagtcttacttcctcagcatcaggtaggcaagaacaacagtcgcacttcgac

SEQ ID NO: 294 - DNA, 896 bp, Zea mays
gcattgacttccttgccctgaactaaagcctgcggacggatggacccattcccttggggcatacagcaatcacactgatgttctttgggaaatcgagtccgagtgattgcaaatgtcccagaagaaatccatgagataaaccaaggatactgtttggtttcatgtgagagaagatcttctcatagttgtcagcctgcagaaagtgatatgcaactttcaggatgaactgaaccgaccatgtaacaattgagtaccatatctccataacgaaatggcgaatgggagtgctacagaacatcatataggttggactaactggcagggaacaaataaccttttgtagaagacttggcacaagtcaagcatcaggcagaagtatacatacacaatcgaatgcacatgaaaccatgcaggctagtgagcatgatgcgaacctgtgcagcatcggatatcagcagcaacacaagatcgctgccggagacggtttcccaaatatcgcccaaggttccgctctcttcggtgaatccagcggcacgtgcttcctcaaaagatttggatcctttccggagaccaatctggaagcaaaaggcagcagcatgttggactgacagaacagtgccgaagtatatacaggaggcagaatatctgggtcatgattgccatgtccaagttgcacaaaataaaaaactgtaaactctctaaaattgaagttaaaaattcgaagaaaagaagccagatgaaaaataaagtagactttttcaccaaagcagagtatcggttttgttttgtttttgtcaaaaaaacatccaacaccagatcttttagtcgtggtggttttgcatgtatgtattatcgttacgacaagggaagaactaaccttcacaacaatatctgac

SEQ ID NO: 295 - DNA, 255 bp, Zea mays
ccggggatgcatcgaagtagtgagtaaacaacatggatttttggatccgttttaccaagatttcagacaaagattcaggcggcttttgggaacttctttccggcatcttaattttaagcttgcaatgagtgtcttggcttcgaagattgattactcaaaccatgagccttcagaatacgataataatagcacttcaaagttattggaagacatgctatcaccacatgacatgaaacgactagaggcgtattcaaa

SEQ ID NO: 296 - DNA, 692 bp, Zea mays
  misc_feature (369)..(376): n is a, c, g, or t
tgtctttcggttgaacttgtttgaagccctgcccaacctggacattttttagtttaggtctaaaaagagaaaccctagggttgtttgaaagggcttgtttctaggcttggcatttttgggtcagtgaagcaattagtatggttgtggatttgcatttggtactcggcattaggtcttatttttcgcgagcgctaactctaattgtattgctgaaacttaagtgttgcttatatattccgttggtaacggatactattctgcttcaattcccttctacattgtagattcgctacaggcttggctcttggctagccaagcctgcagtttaggactttgccaaaactgcatttgccatttaacaggcttctnnnnnnnncattnnctctggaggtctaggnngtggnagacgtcgcagcaggagcaggagccgcagtcgcagccctagataccgcagnnntccgagctataatagaaggtaaccatttttttgacttcttagatttcatcagttgtaaattaactctcaaattttgtcgctcaacctcaaatgctgcttatatctttccttggtaactgatactattctgctttaatgcccttctattgtcgattcattatgggctttacttttggcnnnnnaagcctgcaatttgctactttgnnnnnnnnnnnnnnnnnnnnnnnnnnnnttc

SEQ ID NO: 297 - DNA, 651 bp, Zea mays
tgttgtgttttcctaatgctaactgctgctattgactttacacctatttttgcagctactttcatacctattacttcaatttttttgttatttcagcaacaaatcagcaagaatatggagttttggagaagaaatgggcaaggttattaaaacgtcgtttaaacgataaattgtgattacagggtagaacgtccgatcgttttaaacaaaccgcgtagaacatccgttaaacgtgttttaacgtcaatttggcatcgtttaaacgttttaggaccttttaccttgttgggcctcacaaaggcccaaaaaactaggtcaagacaacctcctcccttctctccttcagtgccgcccgctccggccatctactgccacgtgtctccctcctctcctccaccccctcctccctcctccagcgctgccaggtccgccaccgccacgacactccccaatagcgccgaccgcagaccgcctcctccagcgcccaagcgcccgcttccttcctccaacgccgaccgccgccttcagcaaccgagcgcccgcttccttcctccagcacccgagcgccccatcccctctccctcctccagcaccggccgcctctaatgccggcacgccgcccgccagctctagcagccaacgtcgctagca

SEQ ID NO: 298 - DNA, 255 bp, Zea mays
tcatcctctttcagctttcctcctgtttcccgttaccttggatatgccaatatattgggggaaagaggagcatacagatctgtagttgtaaaagaaaaaacagttagttaccactgtatcgcttttgatacgagattcagattcaataagccaaaaagatttcacatgtacccatcaagaaaagctattaatcttttttatttttctgaagaacgagaatataaaaaaacagctaggttctgtggccttgcaaaa

SEQ ID NO: 299 - DNA, 394 bp, Zea mays
ggcccaatctttccctccctcatcaacaagcttagcacggcacaccttggtatgatgttcctctccaagctataccctataatcgcaggttgctccattacttcgcttagttcaaccttcagcacatccaggaagaaccggatcttcttctttatgatctcatcagagaagttaactatattcggctgtttcttgaacgctctctgcagctcaccctcggacaccccgaagctccggtaaagggccaccctatgcagccacttctccctactaatgcagcacaaggcgcggatgccgtaaggaaagcgcttctccgttacgcccaagccgagctccttgagggcctcgaagatctggcatatgcggtcgggcgacatcatgagcacgcccatct

SEQ ID NO: 300 - DNA, 244 bp, Zea mays
tgtttaggcaccttataactataaatattcttagcgtttgattcattttttccttttggggctgacctaacaacaaatgttagatgatgacactatctgctgcctatctgagtgttgtttgggttcctgactgagcattaccattgtaacgagaaagcgatgtaaaagcccttgctgctgtgctgacgaaagaggattcctttccaatgttacacgatgagtccggttgatttgtgtaacatgt

SEQ ID NO: 301 - DNA, 557 bp, Zea mays
cgttggcacagtcacgtcaatcgatgaacatcattcagaaacattacccagggctgatagcagccgcgatccttttcgaccctccaaagatctttgaatccttttggaaggtacggtgttccatagtgcaattctgtttctaagtttaacagcaggtttactagttctggcatcggttgtcaagcatcctgaactttaagccatggactttgtctgaaccagtacaagcttggtcctgtagaaattgtgacatgccatttcattaactgcagtgtcaatgtataatgcttaaacctccttttttttagatgctaagttacttcatcgagccggagctggaaaagaaggtgaaattcgtgtacactgacaatcctgagagccagaggataatggccgacatgtttgacatggagaagctggagtccgcatttggtggccgcagcgcgtctggcatcgacgttgccaagtattccgagagaatgcgaacaggagatcagattaggggtcttcgctaacggcaaatggaatactgctatctcacagttcgtcaagaaact

SEQ ID NO: 302 - DNA, 449 bp, Zea mays
ttctcatctcaccgcaccggtaatggagatcaagcagagcagctagagcgtactactaacccgagcatcgcgaggacgaagatctgccagaagattggccacgtcatcttgggcctgaccttcctgcacgcagccattaattttccacagtcgcatgcatcgtgatcagtcatcatcagtgcgctagctttagctcgatagatcgccagaaatcagaaatcagcgacgaggacctctcgaggacgagggcgaagggggcgatggagagcgtggcgaaggcgtggcggtagacgacgagcacgtagtggctcatgccctggttcagcgacaccttggtgagcacgttcatcccggcgtagccgaactggagcgagatcatggccacgtagggcttcgccttctccacgaacccggcgcaccgcgccatggagatccttgcttctcactcg

SEQ ID NO: 303 - DNA, 364 bp, Zea mays
tgaggaactgcaggaaggccatccctccgagggaaggaggagggaaggtgataatcatcgacatggtggtcggggctgggccggcggacccgaggcacatggagatgcaggccctgttcgacctctacatcatggtcgtcaacggcatggagcgggacgagcaggagtggaagcagatcttcgtcgaggccgggttcaccgactacagagtcacgccggtcctcggcgtccgctccatcatcgaggtgtacccttgaacgaacgaacgaacgaacgtcgtctggtgccatgtgtgtgtgtttgtgtggagggagggtcatcctctattttcttttttgttttgtttctgcattctgaagacacg

SEQ ID NO: 304 - DNA, 255 bp, Zea mays
ggtcaaatgcagatatgtggagcatgcaaatgcaattcctaaatggactgggttactgcctaaatctggaaagatggttagtgcttgaattctttataaccatcgtatggtcttacaccattagacacacactttttattctctgttatggtcttcactaccattagaaccgtgtaaccatgaaatattattcttggtgctctaggtaattaatacagagtgggggagcttcaaatccaacaaacttcctctttc

SEQ ID NO: 305 - DNA, 761 bp, Zea mays
  misc_feature (62)..(65): n is a, c, g, or t
gccggccctcactccaccttggccgctgttctcctcaccgcgcgtgctctctccctctctcnnnntcggcgccgactcggctaccgccgccgccttacggaagcttcccaggtcttcgtcaggcttttctccggcaacgagcaccctgcacaatcgtgaacctgcacacctcaacgatagatggcggggttcagaagccttgcacccaagaccaggaacctggtggtggccgggggactgtctgccttcgtgctcggggtctactactacaccatgagagcggtgggaggcacggacgagctccaggtggcgatcgacaagtttgaggagacgaagaagcaggatgccgctagaaactctagctccagcgcaggaggatcataattcataaacacgcggacacgccctggatctgctgcaggctgagtggtagatggtacgacgacagtcacgtcgctcagtaggccatgaccatgcgttctgcttgtactgtggaataaataaatgatgggttatggtactctttatctggtagagtttggcagcttgtgctttgagggatgtaatagtggccccgatgtttgtattttcatggcgagccggaaattttgtggagtatttgcttgtgtgactccctttactctagagcaacgactgaaatgcaaacagtccttctatgcagaagcgcaagcaaacatttgatctattacattacgatacatcaaaagtttgaagggcgcctttttataaggacccaagtt

SEQ ID NO: 306 - DNA, 470 bp, Zea mays
ttccccattccgcctctctagctggcctgtcgaggaccctcgccatcctccccaggcgcaccaccaaacggtccaaacccccagcggcggcgctaggatttgagtctcagcgcgccggcaccgccaccgccaccggcggtgatggcagggcaggagcgtaggacaatcgatctggaggagggatgggcgtttatgcagaagggtatcaccaagctgaagaacatcctcgagggcaagcccgagcctcagttcagctccgaggactacatgatgctctacacgtgcgcctccttctcctcttactcctcctcgccgccaccgtctcctgcgcagcggccgcttggtttcacaccgcgatccgcctcgatccggttctgatctaggttttggtcctcttgtcgcaggacgatatacaacatgtgcacgcagaagccgccgcacgactactcgcagcagctctacgacaagta

SEQ ID NO: 307 - DNA, 726 bp, Zea mays
tcatcttctactgaaaaaaccaggagaagtggccatcgcatggcatgacacacaactttgtccatcccttttactctggcatccagcctctgtgacctggacgagctaccggcgcgtggagcccagaaagaacggagtgtcattctcttacgccactgtgaccgccctcaaccccccccaaagccaatgaaaggcggtttagtcgcttcctacctcacttggtgatcgttctctctttccggttgatggttggcgctgtttatttttagcatcggttggactatcagactaggctaagggcccctttggtagggcttatttttcagcttcggctctggctcatgcaaaagttgtgccaaacacctctttttcaaatggcttcaccaatgaagtgcttttccaaaatgaactagagggcatgagccaaaaaaagtggctcacccggcttcagctcacgtcatttttgcacaatagccctcccaccagtccaaattaatttttttggtcatgccctcaatccctagccacgcacaatagccctcccaccagtcccaactatacaagggtctttctaaaaaacaacttataagccgttttgccaaatgatttttcagaatggctttggctcatctaaagaagtggcttcacctcgtgagtcagagccaaagccgtttttgtagaagccagagccctgccaaaggggccctaaaccttgccttagtttaa

SEQ ID NO: 308 - DNA, 426 bp, Zea mays
tggagagggtgagatcacaaaagctttcaaccgccgggactcaaagctagaaaagccatcgccgccaactccaagaccggcccgtccaacttccaggcattcccctttgacgccctctgctagagtggcaccgatacctgcgaggagaaaatctgtcacgcccaagaacgggctttcacaggtggacgatgacgcgaggagcgtgctcagtgtgcagtctgagcggccaaggaggcacagtatagccacctcgactgtgcgggacgacgagagcctcacgagctccccgtcgctcccaagctacatggttcccacagaatctgcaagggccaaatctcgcctccagggttcagcaatggccaatggcgcagagacacctgagaaaggaggctcaactggaccagccaagaagaggttatccttcca

SEQ ID NO: 309 - DNA, 597 bp, Zea mays
ttgattggtgttaaaccaccatgtagcttggcagagctggagaatttttggagccattttctggatattcaggcttagctgagaatgaagacctcctcttgctgcctttcaggctctcagtagaaggatgttcttctttggatctctcttccacatcattcaccaaagcattttcatcgttgttgatgtcttgcagaggcataatttcggccactggagcaacaacagatggtggttcatcaacattttcttcattagtgatggtgactggatctgaagcaacttccacatcaggctggatatcaggtaccagaatatccatctttgcattgtccgacagattaacattatcaggctcttgattctcaagatgaattggaacttgttttgctcttcttagtggtctctcacattggacctcttgatggttaggaatctcagtttttggactagatgccttcgaggtttcagccacggaattagtaaccttcctaaggctacgtttaaccttctcaagttctgttaactggccatcaggaactgactcagcaggagtgctacttaatttccttggattccgtcttgtcttccattttt

SEQ ID NO: 310 - DNA, 462 bp, Zea mays
tggcaactccacacccagcaaaaggcaagacccctgcaaacaatgacaaatcaacggagaagtctccaaaatctggtgggtcagtcccttgcaaatcatgcagcaagtaagtaggcactgatttatttggtgagcctatttggttatctggtgaaaaatcgatcaatgtattgatccttggtctctgtttgctgtctgtccaggacattcaatagtgagatggctctgcaggctcactccaaggcgaagcatggggccaaatgagttgcaggtccaactgagtccatgaaggagaagcatggtggccaagtgagtcgaaggtccattcgagtccatgaaggtgaagcatgggagatccaagcgagtctggaaagattgtcgatgttagtaggctgtccttattttgtttagagacttgtggctatgtgattgatgtagcgaaacaaggctgtgatgtgttaa

SEQ ID NO: 311 - DNA, 710 bp, Zea mays
tgagaacagagtagtttcttacgaggctggcaaggtaatatttctctgcattaattacttttactggacatgcttctttcaaccgggatgatagttaaattggctgcatgttgcttgtcatcataggcccttgctgatgacatcggaataccattccttgagactagtgccaaggatgcaacaaatgtggagaaggcattcatgaccatggcaggggagataaagaacaggtaactagatcatttgtgttgtagactttttgtggcagcattttctcattccttttggagtcatcaagtatagtatgtcaacatatggaggccctcttttattgagggactgagtacaaacaaagaacactgtgcaacataatattgcatatgattcaaagtgaaaaataagccatgaatcccatgatcactatgtttgctattgtttatctgtatacgggagcacctaatcattcttcattctccttgttggataggatggcaagtcaaccagctgcaagtggtgctagtaggcctgcaactgtgcaaatgcggggtcagcctgtttctcagcaaagcagctgctgctcttgatcgtctgtctagatcacagtagtgtgattgtgcagcaagcaaaacttaaattggttgctccctgtacattaagcctattactgtcccaaagaactcgatgttatctatccgatactggaagtctgt

SEQ ID NO: 312 - DNA, 255 bp, Zea mays
ggcagaccgggacacacaaaaaccccaatgcgagcgcccctggcttgtcggcgtcgcctccttcagccaaaaccctagcttgcatacgcgcccgaacaaagcttcgtctgcgaggggcgtcctcctgccgccagaagctttcaggagcatcagcgatggtgtggttccaatgcgaggactgtggcgagaacctcaagaagcccaaacttgccggccacttccgctcctgctccgcgtacaagctgtcatgcatcg

SEQ ID NO: 313 - DNA, 667 bp, Zea mays
tgatagttctgcaagcctgtgtctgagggtactagtatctttcttcggttgttcaccttcattcttaccaacatatacaaggacacagataaagcgaaaaatgccaggagcgaaggtaataaaacttttatcaacaagttctttgatcgtgaatgattactgatattgtagcactgtgctatacctaggcgaggaaggccacacagcgcattgttccccatcaatgatttgagtgtgatatttgagaacactcctccttctggtatctgtccatccaatctgttaaaagaaaggttcaagttggcaaggtaagtgagattggtcaaggattttgggatggcaccggagagtgcattggaggataggtccaattcctggatattaagtatattgctgaatgaacctggtattgatccttggaataaatttctggacagattgagatatatcatcatgtggagttcaccaaaggagactgggatgtcacctgacagcttgtttcctgataaatccatcatggtaattgctgtcaattttccaacatcagcaggcaggaacccacttaaagagttctgtgacaagtcaagctcaataagtttctgaagatcccacagacttgttggtatggttgaagacaatgagttttgggataacgtcataatttgca

SEQ ID NO: 314 - DNA, 140 bp, Zea mays
  misc_feature (70)..(70): n is a, c, g, or t
agatcgatccttgagttgacgaaaacatacacagctgctagctaatcctgccgtgagtgcaatgcatgcnaagatgacgaagcccgcagccatcgttgttgctgctatcgccgtcctctccgtcagcctgctggctgggt

SEQ ID NO: 315 - DNA, 463 bp, Zea mays
catttgcctcagttttccatgctggaataggagcagtcttgctgctctacgttctcttctggctcaaggtttgccttgtcccttgtgataaactgtacttgcgaaaactggcgctgatgttcacttctaatttggtgcagttggaccttttcacaactctgaagtaccttggcttcctcggcatcttcctcgtgtttgtgggccataggaccctgtcctacctttcctcggcgtcagcaaaacagaagacggcatgagtggacagtgcaagcagagctttcccgatcatcaattcgttttttgaggtggccattccaggccactgggtgtctttgcttggtactctgaccgcaccctagtttttctttagctaacatgcattttgagggaggactcggtttatttcagttggctttgacgaatactgatactaggacaagtaacaggacctcacatcacagtt

SEQ ID NO: 316 - DNA, 580 bp, Zea mays
agaacaagaactcgtcctacttcgtggagtggatccccaacaacgtgaagtccagcgtgtgtgacatcccgccgaggggcctgtccatggcgtccaccttcgtcggcaactcaacttccatccaggagatgttccggagggtgagcgagcagttcactgccatgttcaggaggaaggctttcttgcactggtacacgggcgagggcatggacgagatggagttcaccgaggccgagagcaatatgaatgacctcgtgtcggagtaccagcagtaccaggatgcgactgctgatgacgatgaggaggccgagtatgaggacgaggaggagccagctgatggcatgtgaggggaggctgttaaatgtgaagcctggtggttgcagggaccccgatgagttttgatgtcccctttccgtgttgtcggttgtcgccatgttatactagtacactagcataccaccctcgtggcccattccgtcgatcgtggctgttgatgcttgtgtttccttgttctatagaaccttgcttttggtacggagctgcaatgtggattcgatccttccgtatcgctacttatgct

SEQ ID NO: 317 - DNA, 379 bp, Zea mays
ttccggtctcgagcctggtggggaagacggtggggctctacttctcggctaaccattgcgcgccgtgtatcaagttcacgacgaagctggcggccatctacagcagcctcaggggcaaggcggaggacttcgaggtcgtgtacgtccccatggacagggaggaggacgggtacctgcgctcctgcggcgacatgccgtggctggcgctcccctacgacggcgcgccgtcgcgcgcgctggcgaggtacttcgacgtgcgggagatcccgacgctggtggtggtcgggcccgacggcaggacggtgacgcgggacggccggaacctggtgaacctctacttcgacatggccttcccgttcacggacgcgcagatcaggct

SEQ ID NO: 318 - DNA, 357 bp, Zea mays
tctgtcagcgacaacgatgtccgaaacatagttctatcttatcttatgcacaactgttttaaagagacggcagaggcattcctatctagcactgggttgaatttacctgtcgactatagtgtggatgtggataagcgtaaaggtatttatttgcaggtcaatactggatctttcctacattattatgttttagggggcgctttacttgtcatattgcatgatgaagtactatttgtatgttttgataaatttactgcaatctttcttgttgtgtgttcgaagcaggctctccgcatttgcaggggtaaggcttgcctcggtttatcccttccgcagaccccactaatgtgggagccc

SEQ ID NO: 319 - DNA, 778 bp, Zea mays
gacgccgacgtcgatgtgcccgacggtgggccgcccgtacccatccaccgctgcatccttgccgcacgcagcgacttcttctacgacctcttcgccgctcgcggccgcgcaggggcagcgcgcggtgatgcggccgccggcgccggagtagccgcggagggggctgccagtggaaggccgcggtacaagatggaggatctcgttcccgccggccgcgtggggcgcgaggccttccaggcgtttctggggtacctgtacaccggcaagctccggccggcaccggtcgacgtggtgtcttgtgctgacccagtgtgccatcacgattcgtgcccgccggccatcaggtccgcggtcgagctcatgtacgcggcgtgtaccttcaagatccccgagctcacctcgctcttccaggtgagacgcaatttggttcttgctcgccccattgtcaataggattaaactctaatttctttaggaattgtttcgttctatgccaatactgtacatggcttcgttagactagaaatggatttgtgattttgttttctccaatccgagtgttgactacatcactacaaaaagctattaatagctgaactgctaaaattgctgaatgctgattctgttttctccaatccgagtgttgaccacatcactgcaaaaagttattaatagctgaatcgctacaattgctcaattgttgattttgttttttccaatccgagtggtgacactacaaaaaactaataatagttgaactactaaaaatggtgggctat

SEQ ID NO: 320 - DNA, 615 bp, Zea mays
ggtctcctccgtgatctttgatctcgacggcactctccttgacacaggtacgtacgcacgtgtatccatccacccagcctcctcgccatcccgctcccgctcccgctccgtgtggagtacgaccgatcagatagctgtgtcgtgtcgctgtgccgtaataactgatttgcatgcactgctcgcgcaaatgcgtgcgttgtcttggcgcacagagagggcgacaagggacgtcctcaccgaattcctggcggcatacgggaaggtccccgatgcgggggaggaggagaagaggctggggcagatgtatttggaatccactaccgggatcatcagagactacggcctgccgctcacggtcgacgagtactccaaggcgatgtatcccctgtacctgaaaaggttcctttttcacttcttacatgtacataacgcttgccagtctaatcgcttagtagtaataggctggctcgctttctcctactgtcctactacaggtggcaaaaggcaaaaccgcttccgggagtgaagaggcttgttaagcatcttcacaagaacggagtgccactcgcacttgcttctaactccattaggagaaatgttgatcacaaacttccg

SEQ ID NO: 321 - DNA, 347 bp, Zea mays
ggaggaaatcgaaaggcttaagtgtgacaatgcagctctgacctcagagcttgaaaagaatgcacagaagaaacttgttacagagaaacgaatgcaggagctagaagataagttgatctttttggaggatcggcagaaaaatctgatggcgtatgtcagggatattgtacaggcaccaggatctttctctagctttgtgcagcaacctgatcatcacggaaagaaaaggagactaccagtacctatctctctctaccaagattctaatgctgaggggaaccaggttgtgcatgggagcttcatcaccaacccaccagcttgcagggaatcattcgacaagacggaat

SEQ ID NO: 322 - DNA, 780 bp, Zea mays
agcgaggccagcttcgacacggccgacttcacgcagacgccgcacaccgcggcgggcaggtcggaccggcactggtacacgccctccaggcccgacggcgacggcgaggtgtagttggcgtaggcggtgaagccggcgctgttcgccagggacgacagcgccgtgtccacgtccgcggcgtactgcgacccggggtcgtaccgcccctgcgagcacccggcgtacacgaacgccgtgtagtcgtccccgccgcgcgcgggcatcgtgcacgcgcacagcaccatcatcagcgccaccacggggaccattgctgctgcaacaggcttcgacatgcttgcttccttctcttcttactaattcctgcacctacagctttcgaaagggtgcccaaaggaaagaagaccaacaagaagcaatgaagcagcgaagtgtctctcagtgatgggcttaagggtttatataggtgtataggagtacttacataggtctgttatttttttttctaagtattgtgtggtgccagctcagaagtatcaatacatgttccgaaccggctacaatactgaatttgtgatggataatcgagattaaagacgttattttttagtcatttgcatagtccggtaggctcttagctcgaactgatactaaaacgttccatttagtatagtaccgacttgcaaagcgtccgtatgttgctagttgctacagttttcatttatacttatcattatagtataaaacataaagattttccctggttctatttggtttggtggg

SEQ ID NO: 323 - DNA, 841 bp, Zea mays
cttgcatgcctgcagactcccaatccacgtgtatccttccattgattcaggaagcgttgtagccaggtaatagaagatacaggctgctgtgtgcgtacagtagagctccacaactatgagcttcactatcctagtgaagaggtagttgatacgtatgtccttctctaaacgccagaagaattctgtgaccttggtaacccgtgtcaagcgaatccacagcaggtatcttacttcttctttacttccacaagcctgttacggaattcatcatgagagaattaatcataacagaaaaataaagaaaggtagattacagcaaaagacaccgatccagaatttacgagctcaaagctttctttagcaagagagagctaccctgtagatagcatcccatgggaagcaaccaagaagatcaaagatgaagcttgatttgcaatatcggagggcaatagcggtgggactgtagacgattcggtacgtgtctgggtcacggtatgccacgaaaaatttcacaacaatgtcgataaggaacgcagcctgtccaactgtgtccaggaagaagaggttttctgggagccctctgaagaagccgaattccaggggcgtgaagaaggaactgtacaccgcccaacccagtatgaacttggtccataactgataccatctgaagaaaaatcgaaaagtaattcacaaattcagtaaactatcatccttgtgaccatatacacgtttagacagtaataaaaattcgttgtaggagttggccagaatggtatgaattagtattatacaagcgaagcctcggtgctctccatgaagctcaaagtgaaaaccaccaatt

SEQ ID NO: 324 - DNA, 466 bp, Zea mays
cagtgcaaacagaagagttacataatatttttgaattatttgtttggcttgctttatgtatcaataagtttgactacaatttcatatatgttgcagcttcgcagagaaaaaatttcagataggatgaaaaatctacaagatcttgtcccgaattctaataaggtacatttctttgggggtttaacttgtttggtgcattttcatattgcaactggagctgcaactgcagttattaaaactgatcatcgagctcattatcaaaactttgctgcttatatatgcaacgggaacatgtatttcttagttgtggcttgaaatctgcaagtgacacaatagaattgagtatttattaaatttaaggttcttttttgggggttcacaatttcaagtcttcaactctacaaatacgctggagtgaatgttggatttctttgtgccaacaatagtcattacctgtttagccttt

SEQ ID NO: 325 - DNA, 255 bp, Zea mays
  misc_feature (113)..(142): n is a, c, g, or t
gtggcgctgcacggtagccgtgcaggccgcgctctgcctcgcgctctacgcggccttcagcctcggcgagccgcagctcatcccgcggggcggggtggacgcactcgggcggnnnnnnnnnnnnnnnnnnnnnnnnnnnnnnagcgtcgctggcggcgcgcgcggtcccatcgaccaggagaggctcctgaggcagatggagaccacagcaaagctttatgaagtcaagttcgtgctggatgttactcaactggg

SEQ ID NO: 326 - DNA, 255 bp, Zea mays
tctcgtggctgttgttgatcaaccgccaccttctgcgccgccggatcagttttcaccagaattctgtgcgttcatttctgcatggtaatcttactgctttataccaacgctgcctataaacaccctttttccttaaacaacccgtaggagttcttgccggcctatggacacccttacctgtggttaagctactccacatgttagcaattgttttggcatcagcaattttgatctactcctattttaaaatgccat

SEQ ID NO: 327 - DNA, 255 bp, Zea mays
atcactcctgtgcgcaaaaatggatacagaaaaagaacaaggacgctgtcattacttgaaggtggcgaggggagatcctgctgtcgtgaagtacgaagcaagatctacatgcaggtcatcatacatgctcaggaacggatggtcctgcatcgaagatggcaggaagggaagggcaagataagtgccttagttttgcagacgattcctgaaatgaaatgaagatgtggagctgtttttcggcttaccaataaggct

SEQ ID NO: 328 - DNA, 767 bp, Zea mays
  misc_feature (1)..(1): n is a, c, g, or t
nattgggtacctcgcctcgagccggaattcggcacgaggatccaatcgaaccaccagtccaccacctgattgactagagcaaaagcacaagccgcccacgcatctcgattnnnnnnnnnnnnnnnnnnnnnnggcgcgcagagctcgtgacgagagcaaccttccttccgttcctcgatcgccatggacaaggtgctggccttctcgatcctgagcgcgtcgccggccgacctctcctccacgggcgccggcttcggcgggagctgggcgcggctgtcgtggcggcggggcgcggacgaccagcgtgcgccgtggtggtagcactataatcagcaccaggaggaggacagggagaagcgagacttgcgctcccgcgacggcggagcgcacgcgagcggagggggagcggcggcggcgccaccgcggttcgcgccggagtttgacggcatcgactggttcggaaccatcgtgtcgcgctgatcaacaatccgggctcggccgacgcgccccccgagttaaccacgtgaccaatcctgtctactatgttttttttaccttatggtggattaattgtcccaacacagataattgggactccgcgtgttgtacatacagggaactgctcaattaccaggtgggatggggaacatttatttgttcctgtcctctgcattttttttctgtaccgaaatggatggatggtctccaacttgaaattgagtccctcagccccaggtaatctggcggtggatgaacccaagccgaac

SEQ ID NO: 329 - DNA, 658 bp, Zea mays
tgctttcagtttgcggcatcagtcggtggcgcctgcgatggagtctctgttcggccaatcgcagcggaggaggagggcgggcggcagcggcttcgaatcgctcaagcgtctgggtcacatctcacccgctgtgcagtcccacctcaaacatgcaagcccctcgtctccttctccaatcccggctctcttctcacgcttggctttagctgatcccgttcccgactgcaggtgtacctcaccctatgctccgcgctggccttctctgcactcggcgcgtacctccacatcctcctcaacgtcggaggcgccctcacgaccgtgggatgcgtggcctccatcgccttcctcatctccctgcccgcttcacgggaccaggagaggaaccgcttggcgctgctcatgtctgccgcgctccttcaaggcgcgtccgttggtccgctcgtcgaccttgttattgacttggattcgaggtcaatcgtcgtcactcatccattacattacacaagaatgatgagctgtggtctgctttgttgatgaaactgcttctcatgtaccaggattctcgtcactgcgttcgtcgggacggcagttgcttttgcatgcttctctggcgctgccatcatcgccaagcgcagggaatacctgtacctcggcggct

SEQ ID NO: 330 - DNA, 458 bp, Zea mays
cggcggcaatcaagcagcagcgggatgatggagggaccgttccgattcttgccggccgaagtgaaagagatggaggagcgcctgttcccggtcaccaatcgcaggctggatcacatcctcatggatgagctcgctctgaaatttagctgcttccggcgccgtgctggcatggttcccgtcaagccaaagcaggtatgtgcatcgacgatgttccgttccacatatcaatttcaaattccctctcccatgaacctgcggtttcattccgatctggacatgcatgcatccaggtgctcaactggttttataacaaccgtaacaagacttctgccaaggtagcagccagggaagcacatgctccatgggagttttgggccaaccatcagcaagctagagctagaggaggctcatccatcagcaagctgaagccaaagaaggcgactacgcacgcaggatct

SEQ ID NO: 331 - DNA, 773 bp, Zea mays
agaactggacctgccatttgccaattttcctgaagggtccgccctctccacaatctgctttcttatgaagtcgtggagcctgccagacaacatctgtctcaccttctcaaatgccatttccagcctgtcgagctgcgcgtgaagcagcagtgaaacattaggttgttgtccgcgcttaactttaggcatatatagtacatgttagcaaacaattgcgctgcatgcgttacaacaatcgctgacccacgggctaaccaaaaccagcttatgcaactaaactaatgcatgatgtgttgagaggccgggtggagagcacttgcctgctctgtggcattgatattgtacatgaagagcccgtagtacaggtcgaaactgtcgcttacggtgttctgggtctgcaaggttcgtaggcgataagagccagcattcgtctcagcaatcaagaatggacaagaccacttacaagttgcagcagggtctcgtagcccttggtgttgattggggttgaactgaggttcagctgtgacagaaccctgcgccaaacaacgtgagtcatttataacatcgcgttaattaagcaggcagatcgaaaacgatctggagtgatagctgcaaaaacgttccagatatatgcaatgttgctgacctccccaattgtgtgcgtgatgaactggctccccgcggcgtagcgatcatgctcgggacacgacatttccaccattctacaaccctacaagaattgtctagaatgaaatgaaaccaccccaaaattt

SEQ ID NO: 332 - DNA, 255 bp, Zea mays
aaggaaagggcaggaagaagtgagtgatgccaaggttgagtggtgtggccgtgtgggagagactgaagctcacatgtcaatacattgggacatggtacctactgttagagtggacacgctaactagttcaacctaaaagtttaagctgatggtagaaggtaatcaatccacttatacatttcaacaccctcactcgcgtgcagtcagagaaaaaggcacaaacgtggaaataaatgggtggaggcataaataaaa

SEQ ID NO: 333 - DNA, 255 bp, Zea mays
  misc_feature (124)..(141): n is a, c, g, or t
cccacgcgtccgcattaatcagttttattatcagtttatttctgctaactgccatattacgatttacactgcattcatattttctctgcttgaagtacatgtaagttatacggaacaccctgtnnnnnnnnnnnnnnnnnntttatgtctttgtgcagtaaatgggttatgatgaacaaactcactaggaagcttgcaagaattccagatgaagtgcggactgaaatagagccatacttttttgagcattctgct

SEQ ID NO: 334 - DNA, 548 bp, Zea mays
ctggtcccactactgcttaaatgttatcagtatcacatacaaacaattattacaggattcacatcaaagcaattcaagggagttcccccaaaaaactaactagtatgcaggtaagaactgttaagagtatgccatcaattggattggatacatttccttgcagtcaactgagttgattttttggtatgcagaagctaaataaaaaaaaaatgattctgctttccattttgtaccatgttagagcataaaccttatttgcttccttcctttgcacaacagctttcaggtacaagctaacatcagtacaacaccaaaagatagattgggcattgcatgaatcacatgcgtatgaagaagccagaagagtggatagctgtttgtgatctcttcctgttctgttctgttctgttcacggtgtcacctgcttcagcgctcttgcactgccggtaggaagtcttacagagatgagagctaaaagggtttcaaagtaaacaggtttgtttcagaaacgcttcacagaaagtggcgcgcagagtagtgtatgtcca

SEQ ID NO: 335 - DNA, 255 bp, Zea mays
  misc_feature (81)..(85): n is a, c, g, or t
ctcgaggcctgcgcgggacgcacagagcaccacaccatcgccacggcgcctgcccctgtcctgctaccttcgcacccggcnnnnnccggccgggcccctcccactccactccgcgttttcaaaaatcaaaaccccagccggaacagcgaaaccccttccaccatagcccgtaggaaacggagcagcggttgcggtttctctctccgtcgctctcannnnnnnnnnnnnnnnnnntatccggtccgcaaggcgggc

SEQ ID NO: 336 - DNA, 791 bp, Zea mays
gatcctgtttagccgacctgcagcaacatcacctccagcagaaaatgctcttcctttgccctgaaaacaggtgatgattaaatacaatgaaaaataataagatctatagaaggcatcaaggcaatgccacaatagtgattaccgtatcaaaaagagctaaaaggaggaaattagaaataaactaaaccaaagggggggggagttgaaatcagctctgaagtaagttttgggagaagcagccatgattggcacatgaaacgttagattctgaaaaaaaaaatcaaaaaacgaagcttttagccctcatcaccaaattatgttaatcttactgataatgaatcagaaaaacataaccatacataagacatcaagagggtcttcatttaaatgcctaacagttcaatcttaatgcttcaataacttatatttacacaaaaaatctagcgtacaaacattccacattaggatcaagtctaaacaattcttcaaagccaagttcatgtaggacaacgattaaacaggaaccacatctaaatcacaggtaacccctactgatcaaacatacatgcatgctgaattactgatgttgctagaaactcatgcctgtcagtctgtgtgcccagtcaaaaattctttagccttccttttaaaccaagatggtacagtagtagcggcatgattcccaaatgatagagcaagcaagagatgtgcaggcataacatggtgccctgtgtcatggaccgcaaagagtcttcttgcctatggtgcatacgcctgaactctgacaagcc

SEQ ID NO: 337 - DNA, 255 bp, Zea mays
  misc_feature (10)..(11): n is a, c, g, or t
ctcgaggccnnggccgggaaaagatggaaggaggaagaggaaggggtagaggagatcctcaatcctctccactcctttctctcccagctcccaaaattcccctcctttctctcccagctccaaaatcccnnnnnnnnnnnnnnnnnnnnnnnngtcgtttccaagccccgttgggnnnnnnnnnnnnnnnnnnnntggatataacctcgcccgctacctccgtccgaatgcagggccttgatatttaatgggctg

SEQ ID NO: 338 - DNA, 519 bp, Zea mays
gaaggtccatcgcaaatccaacctgcaaaggctgaagatgaatacgatttcttgcacgacggcagaaggcaggccgaagccaggctacagagagcacttgcccgggtgcactctatgtcacagtacccagaagcgagagagcagtaccacaggcttacaacctgcgttgctgaaatgaaacagtccagggtatggtttgatcgctcgctcctatgctttcgaagcttgctgcaggacgtgttttattttccctcggggtttgatcatgatttgatgctggctactttggaacagatgatgcaggatgagatgctaagccaggcggctggtggtgcggacgatttcatggctggactggaggacttgatctgcatagatgatgccccgccccgatgtctgccattcggtgaagctgagcctttttacatcagataggcagggaggcggtttccttgctccccttttgtcagactagccctgtaaatattcttcagctagttgttcaccccgactgtttac

SEQ ID NO: 339 - DNA, 813 bp, Zea mays
gagaacatggtgttcttgtccaccccgccgtggccgtcacccgacaaagctaccaatgcggcgactagaccagcgttgcccgccagcgttgcttcagtgtagttgtagttcctgcgggcatctttgaacccgtcatggcggtcggggccagcaaccatggcacctgctatggtattagggtttggcttcttggcgtccctccacttccaccctcctttgcagccatagtgaacaccgttcttagggatcgaggcgccccggtggtgaacatgcttggggtaacgctttccgaacccaaccacgtagctcatcttcaaaggattctttcctaagatgtaatcgatctgatggaagtagctagttaattagtatatatcagaagttaaacactgctggaacgacactgttgtattaaaatgctggcagcctacctgagtccttgcaaaactgcgaagaacctcaacagagtagaaatgggggccacaataccacccaggagtatccgcagcctcaagatagtcactgaacacagaagcaaggaatgcagcgttgaccacatactggaggggccgtggtttcccgtgatttagctggatcagaccacctgtaatggaaggaagttcagtgaactgtacagttaaagcagaagtgacagtatagtacactgactggatggatgcataaatcacctcttgttctgttgaaggatttaaagactggaaggtaggaacacatgataatgctagtttgattgtggaacgtcctcaagatctcttcgtatggatatccaagactaaagaaaagcctcaaccc

SEQ ID NO: 340 - DNA, 255 bp, Zea mays
  misc_feature (1)..(33): n is a, c, g, or t
nnnnnnnnnnnnnnnnnnnnnnnnnnnnnnnnnttcattgtcgacgatgaggtcattgtgaatcggcaccccaagaagaaggccaggatagggagtgggagggagacagtggtggaccctcaagtatctattgaggaagagacatggccagaccttgagtcagactcatcggactttgagtttgcgacgtccgatgaagaacctaacaatgttgaaacacctgtagctgagcagactacagtcagaaaagggagg

SEQ ID NO: 341 - DNA, 419 bp, Zea mays
cgtcagcttgcgccaatggcgactgcacttctctgttacctggtggctcctgctcaggtcttgcctggcctggcaatgtgtcatacgcgttcaacaactactaccagcagcatgatcagagtgaggatagttgctacttcaatggcctaggtttgataactactgtcgatccatctgtcgacaattgtttgtttcctctcacaattcacgtttctgccgccacttctttgcacctaacattgcccgtgtcgctgttattagttttgtggttttgcattgtctgtatatagtctgatgaggctatagattgagccttcaaaagtgtttcgttgggcctcttgttgaatgtactccaaagtctcggtgcaccatcagacgtattcctagaatggcacaatctggctatcagaactggtagg

SEQ ID NO: 342 - DNA, 592 bp, Zea mays
tgacggaattttcacttcgtaagcactccccatgtcacagcctctcagtttgttgacccaaaccttctttcccttatagacacccctgaatgaattcgcaccaaaccaatcaacgaactcgatctcctcagaacgcagcatccagcgaccaagctcctcgccaccggagcgaatggtttgccattcatctactgagacgaagactgatgcctgtggtaggggagtgggaagctgtggtctgtgcgccggctcattgtcgagtacccggtcatcatctggatcaaacatcgactcctcatcgaagtttctcgatccttcttcttgacaaccgcagagcccaaatggtagcttcacaccaccactgttctttctttgcttctttactaccgatttcagcgcggactcaacctggttcttgaagagcgcctcgttcccagtctgcacaagtatcatgacgacgccaagtgtcagccctctcttctcaaagatctggatcttcttgcaacagaacgaagggctgtctagtgatcctgacattgactgccatgagagcggggctgtgcaagcaaatgttagctggaaaacagtgcct

SEQ ID NO: 343 - DNA, 255 bp, Zea mays
cttgcatgcctgcaaagcactccattaagtagcagttgtcaaggaggtcatcctcgttggccgcgtggatgcccagagcagcaagcatatcaaacacgtaggagaagtccctttcttccgtattcctaaaagcatgggattcccccgtctcaggaagtgatcgacgtgctgtctcgtcatcactcaatatagaaagctcgctgccatctctagtgtctgtagtctccctcttcagaagcccaagttgcaatctga

SEQ ID NO: 344 - DNA, 729 bp, Zea mays
tgctgaaaaccgaactgatgtttgacatggatggatacaagtcaaaaatattaagctcaagatagtaggtagtttgcctagccattttgaactagaagcggtgatctggttgttggtaataggacgccaggccggtcatagaagtaaatggaaagaagaaaactacgaattaaataaaggtgatggtaccaacaccaagttagagacgtctacagctgctcttttccagtttatcagagtactggctggacagatgtaaatctagcaaacctccatgttgatcattgtccggcttcaagtcattgcctgtagattaaaacatttggtgtcattgtcgaagttctgttcaaatgctatatggtacaagtgtacaactacacccttgatttgtttaacattgcatcaaacttagcttactgcttaccttgatcagacaggttcacaggtcaaggcccaaatgttataagatagcaggactgatcaattagctgccaccatcgatggaataggagagattacgcgtgagaccaagaacctagttattcttaggcttccatgtagaaattgcatagagtatccttcctttccagccttgtaggagccatccgttgtcttcatctatgacgaagtccttgtgataaatgtcttttacttttgtccatgggttttcatcacaatttcttggtttaacggagactggacaaattcagccctgaggttcttacctgc

SEQ ID NO: 345 - DNA, 255 bp, Zea mays
tgaacgcccttcgataaagatgactggggagcactcaaaaaaattgaccactctagagctgtgccaggtgttttttcttcttaaactttccgctatagtttgtcagtagctcatgatatgaatgtgtacaggttgcaggggatgacttgttgatgtctgatcctgaacgcattaagcgggcagttaatgagtacacttgcaacgctcttgttctcaaggcaagtatcaagtcatccatataaattctttcttatt

SEQ ID NO: 346 - DNA, 756 bp, Zea mays
tccgttcccgcgctgcccccgccgctgccctcgaggagcctggcgaagcagaggagcgcgcgggactctgtaatgctcttgcagacttcgacgtggccgcgcgcaagcatccacagagcctttgccgccatggccttcatctgcgccttggtggccgggtcctcggcctcgcgcgatccgcggccactagaaccgtttgagctcaggctcagctggggtcttgcacccggcggcgtgactccattcgttgtcgtgttggtcgccgccatcgcggattgcaccaccgattgcatctggttcggtcttgcccgctgttggccagtagaagtttccaacgggttcggcggcacattggttgaggaaccaggctcgtcttggcgcacgcttcgcgatttggcaagcacaacggagtgaagattgtcgagcgacgtcgtggtcgccgctgccgcagtaacggcgttccgggagccgttcacgccgatggaatacctgctatgctccttgatggttccggaagagaggtgggtgacgagatgccggacggcgttgctctgcgcgaacaggtcctggcacttgtggctgtggcgcgccagcgacgcgatggcgtccgcgaccgcggcctgcacgcgcatcggcggctccttgagcgcagcggcgaagacggagcacaccccggagtggaggagcttgtccaggctgtcctcgtcgcggcagaggaggccgagccgccctgggccgcgggcctcctgccccgcg

SEQ ID NO: 347 - DNA, 255 bp, Zea mays
ttttttgcaatagcctcttgccttgctcgttcttcttccatagacaaccgcagggcgagtgccaactctggatctacatttgggtccacaccaaattcaaatccagatgctccagtagctacggcagctgctgcggaagcagcaaaaccacttccaccttcttcaccagtaaatataggagtgctggataaagaaagaaaaataaggttaacatatatgcagcataagaaaacttagaacgcaggaacatggcac

SEQ ID NO: 348 - DNA, 255 bp, Zea mays
aatacgcttcctccgcgaatgatggaggtgatggcagctggaaatctcagagaaagaaaagagagaaagaagatgatgaaactgacctcgaaaatggtgatccttcttctacatcaaagaaaccaagagttgtttggtcagttgagcttcatcaacaatttgtgaatgcagtcaatcacctcgggatagacagtaagattttgctacttgtatttgtagttcttacttttattgacaagaatttgctgtaaattc

SEQ ID NO: 349 - DNA, 725 bp, Zea mays
aaccagaactttcgttgcccacatctgccaccagtgtctttttatgcgtagtcccgctatcactgcccggtgtcccgttttcgtcgccagcatttgcagcggaggacccaaagaaaaagaatcttctcatatttcaggaattagaagtacggaagattcaccagaaattggatccctgcatgaatcgggtcttggtaagcaaagcattaactatatgaaatattatgtagcaagctgtactgtacatctgaagtaacaagacaacaaatgctttctttacgaattagccgagaactaacaaaaatattaacaagagacttgggaaacaaaatcatcccagaaaatgaaaatctacaaggattttttttttgtaaagatcgcaaggaaatgttcagtaatttccacataacattggaaataactagctgtcatctcccaactgaagttgcacatgtcctccacgccagcatctcactacctcgagtaggaaatcaatcgaacatgttacacgcacagccaccggcaccaagtaaaagcgaacagtactcgtttggtcctcgatcgtagggcaccgaaccgaaccgcgcaaaattcaatcagaagggcaactgaaccaaaaaatcgaaacagaaccagacattcgcctagcgagccgtgtcccgtcgatcctcgatcccagaaccaccggcgattcaaggacgccccaccgatccttgacccagcgc

SEQ ID NO: 350 - DNA, 255 bp, Zea mays
agaccgtcatggcggccaatccgggccactacgtcgcggccgtcatcaccaccgcgcagccggacgcgggagacggagcagcctcttctgctgccgcgcccgtgaagcacctcaagctgctccgccccgacgacacgctcctgctcggccgcgtctaccgcctcgtcagcttcgaaggtactgtgcgtgcgtcgtgccctggactggcgccgaacgggctcatacgttacgttgctggccgtgttaggcccggcc

SEQ ID NO: 351 - DNA, 862 bp, Zea mays
  misc_feature (800)..(803): n is a, c, g, or t
ttcttgacttcagcagctaatccttggacaacaggtaaacttcaattggaaagggtaaatggttaatataacagcaaatgccagggccagtgatgcaattatgttatacagcatactctgggtgcaacttcgatattttgctcacaaaagcttctagatcaagtgcttcatcatagttcccattccgtatacacctacaagcataagcctaatgattgtcatgcataattcagcaataatacaacagctgaaactatgcattatgaaaaacaaatgacattgtcattaacagccgactcaaacataaagtaaagagacaagggaatggtaagataaaagttccatacgtgtccatcaatggtgggatttcaagcaagtcaagcaatgtagtgtggttggctagtaatgtttggttaagcttcctctcctccagaatttgttgtgcagactcaacaaactcagtgcagccagatgttaagtttggtatctcctctatctacaatggtgcatgaacaacatggtaaaacatttcaaatcattcttcgaaggccgcatgaacaatagaagtctataatgttagcatatcatatagaaagaaaaaaggaagtgtaataatgagcactgagttaatcctttcatgaagtgtgtttaataaacatactgttggccataacaggtgctcaagatcatctagttcacatcagaatgttgcaaggttacttctgtcctgatacgcatatttgaagatacaggcactgtatttttttcacgaaataattaggctaactatatggaagctnnnntccacannnnnnnnnnnnnnnnnngtcatttttttattnnnnnnnnnnatctatatgac

SEQ ID NO: 352 - DNA, 255 bp, Zea mays
  misc_feature (212)..(245): n is a, c, g, or t
aggccggccgggccggctatccctactcgaccaaaccagagcagcctccaccgccagcgccgacagctcgtgttcgaccttgcgcacttctccgcccatggctgaacccgaggtaggggtggccgcagacacagcgatggaaaccgaagctcctgaagcagccggtcagaagagggaacgggaggaagtagttgatcctgctgctgaaggannnnnnnnnnnnnnnnnnnnnnnnnnnnnnnnnnagaagccgaa

SEQ ID NO: 353 - DNA, 255 bp, Zea mays
actaggagtattgtactagtatattactactgtgctagtatattactgttgtattagaactctcccctttctttgcacaatgaagcgagaccatcgtctcctgtgagttataacacaggttttcatggtcaaacaaccatttgccggtgttgccacagccttccatccaaacaagcaagaagaaatatgcccatcacctagacagcgctagcattgctgcttctcttgaccttgaagtagacaggatctttcagc

SEQ ID NO: 354 - DNA, 527 bp, Zea mays
  misc_feature (18)..(18): n is a, c, g, or t
gtctcctgctgggtttancgtacagagcacgagtgaagattttcatgcacagttcatccggctcacactcctccaagatcttatgaatcgtnctgagcttatcgccgtcttcgtccttctggtaccttgcaagctggagcacggtactntagtagcggccggcgagggaatactcgataggctcccnctgtgggtaacagtattccttagcccactgggcgctcctcgtggtagtgacgatnattgcatcggtgccacaggctagcagactcaaggcgtttctggtctcggtccatgtggatccgtccatcttctcattgactatgaggatgatcagggggcagacatagttcaagtgttccttgatcttggtcgctagcgcgtagctcttcanccgctctttcagcttctgcttgatcttggccatcttctgatcaactgtagtctcgaaaacagctttttccgttcggaccaagttcacttgttggtacttgcatgattcccttactgtagtcagncgccgttcg

SEQ ID NO: 355 - DNA, 557 bp, Zea mays
ctgctttgagccatgactggtggcttcattcgcaggccagcttggtgtcgaagctgctcaggcagatcgcgaaggcctggaaggctgacagtgggtaacggtagtccatggtgaacatgtccttagccaccttcccaaattgcaggatcaccttctcgtgctcgggcggcggcactggggtagcctgtgatggggttggtgctccagcagcgggctgcgtggcagcaacgagctggaagttcttcacagatgcgactgtcaccctgcccctgaaattgaggcaccagcactgcagctgctcgtgccaccttggggccttgttccggagaatcaatggcctctccttgcactcatcattgtctcccgatgccgtgtctccaatcctgcggctgctgctcattgagatgtcggagaatcttgagctgctgaaacgcatagaccggtccacaacagatgacttggagaaggaaatgctgccaaatgactcatcagtagagcgagcaaggatgctgtccagctggctgggaacactgccaccagcctcaaggcacgaggta

SEQ ID NO: 356 - DNA, 801 bp, Zea mays
tagtcttggatactacatttttctactgatctatggttgtccaaacttattaccatttcatagaagtgcatctgatttttttttaaaaaaaaaattgaggttacatttaatttgtgtccttttgttactgattaaagctcattgttgtagaggcactggaccttctccaagatccaatcatattgctacactgtatgatgatagggtccttttaatttttggaggccactcaaaatctaagacacttaatgacttgtattctttggatttcgatacagtaagtttgtgggcatccttgtttcagtatacctcagtacattttgcatttagcaggctactcattagtgttttcttcctgacagatggtttggtcaagagtgaagacccatggtcctcacccatcacctcgagctggttgcagcggaactctctgtggaactaagtggtacatagctggcggcgcaagcaagaaaaaacgtaagtctcaagctctctctcaatttggaagatgagttggtgatttatttcttaatgaagaatacatgtattgctaatgatgaccatctagcttcttttgcaatttctaacaagaaatcaaattctcaggacatgctgaaacttgggtttttgatgttctacaatgtaaatggtctgtttgtgtagtgccccctagttcatcgattactacaaaaaaagtaagtgtttttttttctttctgactctcatatttagttttgatagtatctcgtactcgctactttccattctacaccactttcatctaattagccagaaaactaa

SEQ ID NO: 357 - DNA, 548 bp, Zea mays
cctttgagcatgccgcactgcctttctaattcactttttttgttggcagggtcgtctggctttccatgcgctgttcagtagagaaaagcttccgcaaagcttttctcccccacaagcagaaggctcgaaggaagttggcaaggaggaggaagatgaagtcaccacagtggcagttgacctcaacaagagtaccacaagcattgatcatgacctcgacacaattggtgagccaagggcttcctttcccaatgaactgtcgcccctgaagctgaaattgcgccgaacaggcttcaaaccatacaagagatgttctgtggaagcgaaggagaatagggtgccggctagcgacgaggttggtaccaagaggattcgtcttgatagtgaagcatcaacatgagcgatttctacatggttaccgctcggacttcctctgccgccatgaagtttatgacaaagttgtatttacatctaaactagtataggacttaaagaccttgcaatgcaatgcttcatgaaactgttgtgtgctgtattttatactgagaaaa

SEQ ID NO: 358 - DNA, 350 bp, Zea mays
tatataagacggggtgctttgcattgcgtgcgtgcgtgttcatgggatcgtgttaagacggaggatgcactcatggtcccagagagagagagcgagagagagagaggcagcatatatgcatctttcatcgttaaaagaagctaggccatggacagacaccagcagcagcagcagccaggaaaaaggattaaaggtgtagcatcagcagcagctagctctagggtggtagtggtagtagtggtagagtgaggtgatcgttcgaaagccagctaaacacagatgcaatgtgtgtaatggatggagttgttgtgttagatcgagcgcgtttgcgtttaggatcggaccatctg

SEQ ID NO: 359 - DNA, 255 bp, Zea mays
  misc_feature (1)..(28): n is a, c, g, or t
nnnnnnnnnnnnnnnnnnnnnnnnnnnncaacatcttttatctattatctatcgtgattcaattacacgaggcttgaaacaaacactatgcaatggcatctataaataagtcacgttacaatcgtccggaccaaccccttcatcacaaactacagtctttcggcatcttgtgcatctataccaatcaacctctgtttcatgaacagagtgctatgtttctttttagttcagaatgtaccatatatctaataacag

SEQ ID NO: 360 - DNA, 430 bp, Zea mays
cgcgcgagcatccaagaactgtggctcctttccatggttcttctagcgtcgtcctctcttgtctgtgcaagcatggcggtagatggtaactttcttctgacgtgaatatatacatacatctagtgttcataattctaactgacagaagaagcgcgtgtaactcgattgaccatattgctagggcaaaccaaagaggacatcaacgcaacgaatgtgacgagcatgaacatgacgaggtcgtcgtcggcgagctacaacatgactggcggcggcggagaacttaacagaggtccgtgcgtcgtgagatctggattctactggtgccagaacattggctatccgaccatgtcggagtgcttgaagaactgcgaatcgtaaagcactggagtatagaaaaggaataaagtggtctctgctagcttaatttctc

SEQ ID NO: 361 - DNA, 487 bp, Zea mays
tttcatccaagttgtagagctactgacgaagccatgcctgccgcctacattatcacatacgtacaggtatgggctaactaaacgactcctctgctcaacaaatttcagtcctttgtcccgagctcgggttcgcacgagctccagcacttcgttactgacatcacagcgacacgctcgtgtgctgcagcgggggagcgccgtctatgagtttactccatcatcagctctaggaggaaggagagctgctcagtggctgctgtctgctgacctgtctgtcattggcatggcacatcgtcaagaactcgccgcaagcccgcctcttcctgcgttggagcagaacgcgagacatttcaaacaccaatttttttcccctttcgtagtctcggtaaaactctggatccagctctcgcgatgcagtgttacctgagcttgtttaactgggtagttagatcactgatctccaggaaggatagcttgcttgagtgaa

SEQ ID NO: 362 - DNA, 825 bp, Zea mays
agccaccaccttgagaagacgagctaccagcacctccagattgcccagggattgcatatccaactttgattccataggactgcaaatttgacaaaagatgtataagatcatattcatgtggcggtaacaagatacacactataagcaagcaaaatgcttttgagaggacaaggttcctggcgggcaaatgacatcttcactatcagggaatttgctagtttccagaaaaaaaaacgcttcatctgcaaatcccttgacattaacaagtaaaaatatatattcctcggtctttttcagcataaaaaaacaccaccccaacaaaattatacttaaaagtttattcaattatgacatttcataaaaggctacttctctgccatccatcatataaattaagactacgaggtctaaaggtcttagttaacagcaagtttcaagagatcacaaggataaacataaaggtacaggagtatctgtctttatctcggcaaaattaatggttaatacattttcggaaggacaatcatgaacgtgatacattgttactaatctacataagttctgtaaatggtagttaaagtaatgttgaaggggctcactacattttacttaagcagagcaaaactaatggtttgggagtaaaaaaatacctcattagatacatcgaaaacaccatcttgaatcttcttgtagattgctccagcagtcttaacaaatgcctgcatttaaaagttcatgtgacatgagaaagttggaaatttttttgacttggcgtagatacaccaagaatcaagcatcaccttctcgacattctgtgcggtcttc

SEQ ID NO: 363 - DNA, 255 bp, Zea mays
cctcatcgtggctgctagctcaattggcgaggagctgttctaccgcgccgccatttcagggagcactggccgatattttcctgagaagcacggagctaatgaaggatgctcgagggattgcatcactgagtggtatcgttcctcctctggttccattcgctcagacgttcgcggcagtcatcactgcagccctcacaggaaccctctactacgtcgccacaactccgaaagaccctacgtatgtggtgacaccag

SEQ ID NO: 364 - DNA, 773 bp, Zea mays
gcagtcgccatcagtataacggttacaggccaaattgcaacatccaggatcagtgtaagccagcaatgccgtggcgtcaaattcaaatcgaattccttaacctctcccaagaacggccaaagcctatttctgtgacactggcaacgagtgtagcaacagtcacactggcaagcaagcaagcagcaccagcgtttactcaaagtcgcaaaccaacgaaatgccatggcgaagctattgtggaagctggcgtacgtcgagcaccagcgctacgcccagcgtcggctcgtcgtgtgtcaagtctggacattgtgtaatttggaggatcgcagaaaaataaataactcgtggagtttggacaaatccagccggtccttatcagttagttcaggctatacaaagacgacaatgcacacgtcgccaccaatcttgttttacttggctttgtccactgcacttctgctgcggtatatatatatatataagccgctctgctagcagaagcaaaacatgcatcgggaacccttacaggtagataccacaaccctgtgcagagttcttgcagtctcatcaaggtgacaggaacgacataggacatggaggccaccctgagcccgaagccccatttcgtggtcatcccatggccagccaccagccacatgatccccatcgtggacatcggctgcctcctcgccgcgcacggggcccgggtcacgatcatcaccacgccctccagctcgcaactcgtccagagccgcgtggaccgcgccgggcaa

SEQ ID NO: 365 - DNA, 255 bp, Zea mays
agcataagctcggtaggttactgcgattcctgtgagatcaactatattgtttctgcattggcatgtggtgtttttttttctctccccaaaacacagcatgtgctgacatattaccttgtgttgcaggctcatttgctcatgggtttacatactctggtcatccagttgcctgtgcagtggcaatagaagctctgaaactttaccggtatgctgcacttcttgtagcatgacagtatccctgctcagatgtcagta

SEQ ID NO: 366 - DNA, 442 bp, Zea mays
  misc_feature (433)..(439): n is a, c, g, or t
tttctcttatggagtaatcccagatataacctctccatttttgacatctctttgctctttcaggagttggtgaaatttacacatatactaactaacgttcatttttgcaggtggagaacatttcatttgatttgtcacaatacaacaatgccccaagcttccactggcctgaaagcagcagcaggtctcagctccactgagtaggcagcaatgcaatcgtggatgccttccaaatagactaagttctgacagtcttgtaagggcttgttcggttattcctatcttctgtggattagatgagattggaaaaaaatatgaaagattttgacttgaaagattttgacttgtttgggattaaaacgcacccaattccactcaatctacatggattgagagcaaaaccaacaagccctaatttggaaaaaaaaaaaannnnnnnggg

SEQ ID NO: 367 - DNA, 1115 bp, Zea mays
gccggccgggtcgggactgtgtggggctagctagtcttgcagactcggagatacgactagcttgttataacaagccaagcagagcggtggggaaaacaatggcgggcaatgagggtcttaaggtccttggcctgcaggtgagcccgttcgtgctccgcgtgtgcatggcgttgaacacaaaaggagtgagctacgagtacgttgaggaggacctatccaacaagagtgagctcctgcttaagtccaacccggtgcacaagaaggtacccgtgctcatccacaacggtaagcccatctgtgagtcactcgtcatcatgcagtacgtcgacgagctgttcgccggccggccgatcctcccaaccgacccctacgagcgcgccactgctcgcttctgggctgcctacgccgacgacaagctgttcccagcgtggtacggcatggtgaaggcccaggcggaggaggagagggcggagaaggcgaaggagacgctcgccgccatcgagcacatggaagtggccttcgccaagtgctccggcggcaacgccttcttcggtggcgactccatcggctacgtcgacatcgtgctcggctccttcttgttctggttcgaggcggtgcgcagggttttcgacctggagatcattaacgctagcaagactccgctgttggctgcgtgggcggagcggtttgtagggactgtagaagcgaaggaggtgctgccgttgcccacggcggacatggcggtgcagtacatcaataagcttcatgcccccctgccgccgccatgagttcacaatgagtcgtgtaagtgtaaccaagcagggaaaaaggtaaatggtgcggtgctatggtccaaatttcaaccgaataatgttcaaagcttacatcgataggcttgggttgttgtcatcaaataatgtggttcagtcgtctcctctgcaataaatataaatatgattattactttctttcgaaaaaaaaaaaaaaaaaaagcggcccgctctccaacgccacagcttgcgcccgcaatggcactcgtctccgcctcctcgtcctccaccaccgccgtcacggggctccccaggaatgggcagcgggcgtcgtcctccttcctc

SEQ ID NO: 368 - DNA, 595 bp, Zea mays
  misc_feature (465)..(465): n is a, c, g, or t
gagctctacccgaaccacagaggccgtgcatgtcccggaggcgaccgtgcaggagcagatatcctcgcccttcatggactcgccgccgccgcacctcgacgcccccttcttcggggccgatctgatcccctccggccgcctccgtcggatgcgcgggtaccgtactcccccggaggcctcgaggaagagataatgatgttttagacaagagttttactgggaggaatgaacatgtacgatcgctaccaggattggcgccttgatgtagataacatgacatacgaggagttggtcgagcttggagacagaattggatacgtcaacacagggctacgtgaggacgagattgctcgcagccttaggaaggccaagcacccatccttcggttccttgcgattcgcagcagaagtggaaaggaatgcactatttgccaagaagaattcagtcaaatgaagagatgggaangctagtctgtggccatagctaccatgtgttgtgcatcaagcatgggctctctcanaagaacactgcccggtttgccaaactgccgtcaccagaaattgaaatgcaaactccaaggcacgctggtctgcat

SEQ ID NO: 369 - DNA, 2264 bp, Zea mays
gataggaggggtgcaactatttagtgacgagaagaagcaagcaacggccaataggcactttgtttataggacataatatttacagattcaattttcttttctgcggtccaaacaatccattttttttctattccagtggtttgcaatccggtagttctttcacttccctgttccctcgtttctgcgttttgcaccgaccaaccaaaattcggcacgagggtatgcgggttgctgacttgtccacaacaagcacatcactttcgttttcttttctcccccaagaagatgagtgagaagatgagcaaggagggatctcgctttagaccagcgaatccatccacaaagattccgtgcacctccagcgcagcctccgctgtctgtccccgcggccgcggcaaccgagcaggaagaggaggaggatcccgtcctcccagtctagagcagccgcttgcaaagcgcatcggcggcacggccgtggattccgcgaatctgccaccgcttgctgctgcttctgagactgacaccgccatcgcagctgatggggatactacccagcgacggcgacgacgtggtgcaaatccggcacgccgacgccgccggcgagcccaccgtcgtcaccgtgagctgccccgacaagacaggactgggctgcgacctgtgccgggccgtcctcctcttcggcctcagcgtcgtcaagggagacatgagcaccgacggccgctggtgctacatcgtgttctggctgttgccgcgcgggcggcggcccgtggccgtgccgtgggacctcctcaaggaccggttgcttcagctctgccccgtcgcgccgccattcggcttcgggtcccacgacgcgtacctcgcggcggcggctggcctgcaggacgttccgcctcccgcgccacagctcttcctgctcaagctatgctgcttcgaccggatgggactcttgcacgatgtgacacgcgttttgtgtgagctagagctcacgatccggagggtgaaggtctctaccacgcctgatgggagtgtgctggaccttttcttcatcacagatgccagggggcttctgcacacaaagagcaggagagaagaaacccaggacagactcgagagcgtcttaggcgactccttgacatgttgcgaaatagatcctgctggcgaagacatggtgtcttgctgcctccagtcctgggcctgggcttcgctgacgcctgctgttatagcacagatgtttagcacagctgatcgcgtggaggagcaaccgatcggcacaaggggtggtggtggtggtaccatcatctccgtgacgatggacaactcgcttagccctgcccacactcttattcagattcagtgtggcgaccataagggcctcctgtatgatgtcatgaggatagtcaaagactgcaacattcagatttcatatggccgattctacgcgagccaaaacggaaggtgcgagatcgatctgttcgcggtgcagtcggacgggaagaagatccttgatcagcacaggcagagagcactgtgctgtcgcctgaggatggagctccgccgaccactccatgtggcgttggtgaaccgaggtcctgacacggagctgctggtagcgaacccggtcgaggtttctgggaagggtaggccgctggtgttctacgacatcacccttgctctcaagaatcttcagagacgaatcttcttggctgagattgggaggcacgttgtggaaggcagggagtgggaggtctacagattgcacttcggtgaggagcacgagctctcgtctgcgctgcggaagaagattgtggatgcagtcaccaacatgctgatggggtgggactgatggcactgctgctgctgccgtgcctcggtctgaaacattatgcttagtgttcttccagaaactaacctatttggtgcaagcaaagaagaaacaactgatctctatctcttagatgcagcggttttcagctaatgcagagaccatgaaacctttggttatctaaacatgcactaggaaccggggtcctactacttagtgggcaccagacctgatgtatatgcatatactgaagcatgatcaagagtcatgtgtggcatatatataccattttgtctttgtcagaaaaaaaaaaaaaaaaaagtacacaagcacacaaaaaaaaacacgtagaacagatgttggagatgaagagttgtacttctggatgattacaaaatatgaagtgtatataaggttcagatttaaaaaaaaaaaaaaaaaa

SEQ ID NO: 370 - DNA, 266 bp, Zea mays
gggaccaagccctatgggcaacaatggagatgaccagcgtcttggtttatcaagaggaagttaatccctctcaactgaaactcccatcaaatttggggcatagtgtgtcgattcagttgtaactattatatgtcattttgggttagcaaactgtatctactaatttctcagccttaagccttttgagctagaaacttgtgtaaggatttgttgcactaagtgacatgctgatatttgaactatttaggctgccaaatctgtttttt

SEQ ID NO: 371 - DNA, 1114 bp, Zea mays
cccacgcgtccgactattttggtactcgtgatagtgcttttctgtattgcacattgaacgctaaaacaagtcccatatgaatcatttagtattacttataagcaagcattagataagttccatgcgctttctctgatttgcagctggaacctaagcatgatcagaaattaatataaacttttgcttatagcataacacccttgtttcttgcaggttttgcatggtgatgcagttcttgatgaattattggactgtaccatattgagtggggatgtcattatacgacatcttgcacagcttctaagtccgaaatatgtagtatttctggtactgttacaaaacctccttttctgcaattaaatacccagacgatttcctttcaggtaaagtgagaaaaacggcagagcgactcctaaaaagaaccggagggagtactgaataagcattgccaacctagttttcattcaccattgagaatgtccttctgtattttcaataacgtgacatcaaatgctcctagtgaaggtgcagtggtttagaagcgaactgtaggatcaaactatggtttattaggctgaggagtagggacaatgatgtgcctggcctggttttttttttcttctaaactgcttgctgtagccttgtattttgtagagtaagatatctgttgttctcatgtactgcagtggagatatcagttgcagcacatgacaccaccggtgggatggagaccaaaatactggaggctgcagtgatagctaggcttggaatagacgtgtatattaccaaggctggaacagaacactcgctgagggccttgaagggagatgtgagcaccgactcggaagattggcttggaactattatacgctcttccaaatagatttcttgttgcaaaaacattcggactggtttactggcgattttcccgatgttatatgagcagtgatgagccttgggctttcgatgatgatccagcacgggtctattccctactgtggtcgtagtttggtttacaagcgtcagcctgatgttacaaatgccgttcagtaaatttgttttgttttctgaaaagataagatgcattggtcgcaagagaggaaaaaattgattt

SEQ ID NO: 372 - DNA, 3249 bp, Zea mays
ctttcacatggcctgccgtgccgcgtctctcccacgccactctcgccggtcaccagatcgtcgctcgcttccgttattaacccgttttgtagctgattccggggctcctgctgcccgcggtccacccttcggtcagctaataacgcctccgcctcttcccttccctcccttcccccttgggcttgggccctgcccattaaactcgagcagggcactgtttctacaacgtttgccgttctcgaccgaccattgatggccacgaagagttaagccctgctctgggaagggcactgtttctttagcttccatgcgtagtaggcacgcggcgcacgcaggcaggcaggcgggcatggaaaagggaactgtagcgcccacgcgttaccgaccgagagagagagagtcgagcgagtcctccacgtccgcctgcgccccccaaagaaaaggcatacaagccgtcgtgtgttaatggaaacggacaagtgtgaggaggaggagcaaacgcgtcagccttttttccgttgacgcctcgcctcgcctcgccttgctgccccctcctcccgttctcgaacaggtcaagccgcgcctcctctcgcctgcctgcgtccttgctcttcctcctcctccctttgcccgtcgtccgctccgcttcgttgtctcatccgctgccgttctagcaagcggggcagcgtcgcgcctcggcctgatgctccaccacgctccagtccacagcctccccggtccttctcgttcccgttctcgttgagagcggcggcttgcccggcccccgggtcctcttcacctcacctaaccttgtttctagtttcggtccgtcggttggtgcctcgtcggctgcctctcgctgcttggcccgtttcttcctggggtcgcgggcctggagtacacaagtatataatcttcccggccgtcgtcttctctttctcgacctgcctctcttcgcattggcccgttcccgttcctcgctccctgctgctccttccgagacagactgcctgggatcgaagcacacgataggagagacctcctctgtcgtctgtgcctgggtgagaggtcgggtggtggtggcagtgcttggatgcttcacgcgaggccttggatggtggatgctgcgtgctgctggtctctgcgctgcgcctgaggtactctagagattagtttggccgcatcctccgtatcttctacttcttgaccagggatggagcttgtatacatggaaaggagaaacggatcgtacgacgatggcgcggttccagagttcagtggaaggggggctggaggggatcaggatcaggacccgcccgctgctgcatccgaggtccagtctgactcgcgtcagacaccggacgagaagaagagcaagggctgccaatcatgccgtgacccattctgttcatgcagcggcggcacgccccactcggaactgttccccccacatcccgccaagatgatgatcctggagttcctcatcgcgagcctcaggcacccgacaagaacccacaacgtcagtgatctcgacgacctgatcgccgacggggtcagcccaagggagcgtggtaaacctcggcccttctgagaagaaggtgctggaatctctgcacgccatggtaaacgccaagacgaggccgaagagcccgtcgccgttcatggcgggatctaagctgaggaaaatccggtccaagtcccacgtcataacgcagtcggaaatactgaagctcatctctcctgagacgtgggaagcctcctccttccctggggcgtctccgatgatcggcggaggcacggctgaggacggcgtccatgggaacaagccgccgccgccgtgctgctcgtctgtgagctcaaaccagcatgttctgtcacggtgcccgtcgtccctcagcgcatgccttctacagtgcatatggaaggatggcctgcctcattttgagctgtcactggacgatcatcccattgcggtgtacgctgcaagtccagtcagggtgctggagaaggacagggccttcgactacgtctacatgatccattcgggagagcaaggaagaaaagactggatgggccactcgagcaacgtgtccagacttgtgggcaaggtgaaggtgtcaagttctcttgttcttaacccagacaagtcaggtcttttggaaaccgagtttgttctgtatggttctccggacgactatcttaggcagatgcgcagtccctacagcgtgccgaaagggaagggccttgtcaggagggtgacagagatcatgagagcaggcaatgtttccgctagcccgagacatccctggaagctgggcaaatcttcctctcatcagttcgacgacttggctggagtacttgaaggtgaaatgatcagtgctggaggatcagacctggtgagtctcaaggccgacgatctgccgtccaaccaagaattagcagcgattgtggtgagggaacagcggcgcagacgacggaaagagcctgtttcgggtggctggggcctcaagttccttgagaaagcaggactgaatcgcccataaggcactgaagaaactgatgttcagaacagtgatggtgccacacattgtgtaactgttgttgtccccagaggctaccacggcggcgtggtatcaaagagtagtcgcccgtcaggcctcgtcgagagatggagatctggaggacgctgtgactgcggcggatgggatcttggctgccccatcagagtgctcaacggtgatggatgtggctccttgcctgaaggagagtcacgggacagaaggtcggtagagatatcgatcaagggtgcaaagaatgagacaatgttgaagctcgtgaatatcacggaggacttgtacatcctctacttcgactcgagtatttcaccattgcagtgcttctcaacagggatagcgattgtgcacagccgagcgccagagctttgccccaaactgtgaattgcgcacagccaggcacgcacccgagctttgccccccaaactgtgaattgcaaagcttggtgtagctgaattacaaagattcgtgaagcactatgtacatgtagatagtagaatgggaataattcttgttatttttaacttatataattttgagagagaaaagcaggcagctgttttcagactgcttttaaatcatcaatctgttggtttggccgattgagcggaagctcagaaagatgaatttacaaatcatgtttcgttaaaaaaaaaaaatttttgaatcccttgac

SEQ ID NO: 373 - DNA, 2060 bp, Zea mays
gaattcggctcgaggcgagagcgagagagagagagagagagaggagagagagagagagagaggagggagggagctcgagaggtcaacgagggggagctggcacgaaagctcagggctccttgctcctcctataaacccagcgcacccgccttccaggctcccattttctcgcccgggagccgcaattccatcctgctgctcgagttatatactcacccaatagaacaattctatgcctgatgattgtacttcttgaggtagaagatcatactgaagcagaactcgagcggtgttgaatcgaaatggaaggcaggatgacaagtttggaagccaccatgaaagtttcctcatcgatggcaagcagcatatcaaggaatgctgaccctgaccaacagactctacgccccaattcggtggagcagttttattttccaagacctggtcaatctttgccaggcattcctccattctttgggcctccctcctccagtttgtatcttcctaatgacaatgaagctaaatttggcaatcagtttgaatcaaatccttcacagaatacagattgggatccgcaggctatagtgagcaacctaaccttccttgagcagaagatcaagcaggtaaaagatattgtgcagtctatgagtaatcgagagaaccaattagctggcggttccagtgagctagccgcaaagcagcatcttgtaactgctgatctcactagcattataatccagctcatatctactgctggttccctgctcccttcaatgaagaatccacttcttagcagcaatccagcagttaggcagcttggcaacactcttggttctcctatgggccttggcatgaatgctaatcagagaccaagtgtagacagcaagactgatattcctgacacaggaaaaacttctgattatgatgaactgatgaatagccttaatcctgcccaagatgaaagagatgagatgatcaaatgtccaaatccctgtggtggggaagggtctgaaccgactcccatggaggaccatgatgtgaaggaaagtgatgatggtggtgagggagagaatctcccccctggttcatatgtagtcttgcaattggaaaaggaggagattttagcaccacatactcatttctgcttgatctgtggtaagggtttcaagagagatgctaaccttaggatgcacatgaggggccatggagatgagtacaaaactccagctgctcttgccaaacccacaaaggattcaggtgcagatcatgcacctgttacaaggtactcatgcccatttgtgggttgcaaacggaacaaagagcacaagaagttccaacctctcaaaacaatcttgtgcgtgaagaaccactacaagcgaagccactgtgacaagagttacacctgcagccgttgcaacaccaagaagttctctgtcatcgcggacttgaagactcatgagaagcactgtggtcgtgataaatggctctgttcttgtggaacaaccttctccaggaaggacaagctttttgggcacgttgcccttttccaaggccacacacctgcgcttcccatggaagatgtgaaggtatcagaagcatcagagcaacctcaggacagtgagccgatgaatgaaatggcgaggagtaatatgtatagcttcccctgtagttcgtctgatggtatatcgaacctcgacatgaaaatggctgatgatgtgcgtggttatttctcgccgctgaactttgatccttgctttggcgcgctcgacgatttcactcgccctgggtttgacatctctgagaaccccttctcattcctgccttcaggatcttgcagttatgggcagcagaacggagatagctgatggatgccgccatcgatgatgacgacaacagcactgccaacctttatttatgttgttgcatgatgctagcttggttcttcctctgttcaagagccatttgagattatgcctttatattttccctaaatgtacttacgtttgtggtcattaataatgatcaaggacttctagcaaaaaaaaaaaaaaaagggcggccgc

SEQ ID NO: 374 - DNA, 1832 bp, Zea mays
cagacactagacactacctatctactccaagatccgcggcggcgtgctggcctactctaagatccgccggcgcgtcgtggcggctcctccgcgtcccgcctccgaggccgcgggcggcgtcaggctgatcggggtggcgcgcggcgccggggagcgccccatcgtattcgtgcctctcaaggtcacaccgcctcgcgctcgtctcctctccttgtcgcttcgattcgttcccctcccgctcccgctcgccctcgccatgaatgatgtcgcctgaatctggttctgggggtttcggtagctactacttggccggcttgccgcctcttcttctttgcgttgctgctctgccattttcctattgtagtagcggcgccatgcgagaacgagatgagccaactgcagaggttggcttggcttctgccattttcttctttgctgcagaggttcgcggacgcaggtcggaccactgccgcgcccttcctcttcctgccggccgccgcactcccgccagcgccgtactacttactacctgcatactgtattctgtagtagtatgaaaaggtaaaggcggcgtactgctatagtatctctgcagaatgttcggaggaggagtacaccaactgaaagaggtttaggaaagttgcaccgtggatctagaattctagatgcattagtgtagcctacagtagccctgtacacacataaaggcatttttcttataaaattgtctcgcaaaatgggatttttttgtgctaattatagggtgctttagcgccacctgggatttttttgtgctaattatatgcagcttccatagaccaccacaggcattcccgcatgcagttcgctctgaatgcctcctttcattctattactatccctgaacgacgacccatctcagggaaatagccattaaatgatagcagccttttttaaagtatggcttggtttgcgatgttattatggttcgaaaagttgctctattttagtctcatttattttataaattgcagtactaaactgttttagtctatagttcctttttagatgactaaaagggattaaacaaaaaagacacccgtaaattaatcagtcgttaaacagtgctagtactatgcatttcatcgcgtaaattaatcaattgaatggtttcaattggtgattttgcagcctaggccatggagggggtatcatcagctccagaaatggacgaggctatctcaactgaccaatcaagcagatcaacaaaaagaagggctaaagtgtgggatcatgttgattcagagctaatagatgggaaagagaaggcggtttgcaaatactgtaaggcccacttatcttctgctgcggtgtaaacaatagagcaatgaaagatatgcgagatgctttgggcagccagatgtttttcagtggtgaacacctccatgtgaggtgttcttctcatgtgctcaacatcatggttcaagctggactaaaggctaaggagttccttggtgcttccggtgacaagcgaaaggaagtcaattaactctgaatcagtggttctgctgcaaggttacacacaacattttattgaatggaaagaggacactcggtgaccacaagatcatcagatgatcatttgttgagctctggaactaaatcctctccgcaggtggtaaccaggcgggttcccatccgagttccgaggtcactgtaatgctaaattgtcaagttcagttatctgaattcagtttgagttataattctcatcaagcatcaatgtcaccaactgtgtagaaatactgaattttagcatggagcgtctttataaacaaaaaaaaaaaaaaagg

SEQ ID NO: 375 - DNA, 1954 bp, Zea mays
atgtcgcccatcctgttggaggattgggaatccgccggtcacggcgtgcgtccacgagtcgaccggctcagctccagccgaggctggcgagcccaccgcatcccgctcggctcccgccgcggctgcgcggtgccgcgcaagctcgcaccacgcgtcgcgccggaggcaggcccgccctcccgtcttcgccatctccagcggaaccgcatgccccaaaagcgaagccaaccagcacaaaaaggagcacaaagctctctctccctcccccctctctctctctctctcctgttccccgcgagagtcgctcagggccgcgacgaggagaagcggcagcgcgtcacctctccaatggcatccccgtccctcctccgtgcaacctgtgcgcttctcgagtgctgaggctccctgtccctaagacaagcttgcgcgcgcgatggagggtttttcgagggacttgctgtgcgggattggcaagggggacgcgccgccaccggagaagaggccggggcagctgcgggaggagatggaggaggtggagcttagcctcgggctgtctcttggcggccgattcgggctcgacaggaaggggagcaagctcccccggtcgtcgtccgtggcggccatgctgacaacgccggtggaggtgccggcgccgccctccctctcgcgggcgagctcgctgccggtccaggcggaggcgtcggaagttgagaggaagcaggggctggatggatggggttcttgccgtgagggcggcggtctcggcgtgcaacacgcggcgaggctgccggcgagcgggaacccttcctctgcctcttcggtaggtgaaggacagattttgcaaggcaccctcatgaggactagttcactccctgctgtcattgaggcttctggcaatgatgactggaagaaacgaaaggaagcacagagcctgaagaggctcgaggttaagaagaagaggattgagaggaggaactctctcgcctgcaacacttcaaaagaagctgctgggcagagtccaaaagagatgaatgcaaacactgacaaattagtaagttctgatgaaactattgtcagcgctaacgaaagtcatagcagtggcaagcatctggtgaaggggcttccaccaaagtaccaggccacaattacatcagaagacagttcgtcagcaatgcgaaagaagccaaactcggccttcaaaggaacagccatcactgaggagcagaactcatcatcgtctgttccttcctccggtgaagctataagtagcgtgacggcaccaagtctgcctctgttgtctttggtccccataacagcaacccttggttccagggaagaccaaagcattttaggcagggctggtgcaagggcaaacggcatgggggacgtggaaaggaggatgatgcaggagatgccgggcgtgttcaccaagggcctgtccaacggcagcagggtcgaaggcttcctgtacaaatacagcaaaggagaagtgaggatcgtatgcatctgccatggaagcttccttactccttcggagtttgtggagcacgccggcgccggcaaggtagataacccgctgcgacacattgtcgtcagcgctaccccgaacctgtgactgtgaggccaggcagacccgtgcacaagctatagtgttctgtagactcaggacctgagcctagcgtcagagcaatgtcaccgttagactgtgtggtgcggtgcggtgcttatgcccgattaaattggtgggcattgtggtaacataacctgttgataatgttttgacaatgttttgatccttcctaaaaaaaaaaaaaaaaaaaaaaaaaaaaaaaaaaaaaaaaaaaaaaaagggcggccgctctagagtatccctcgaggggcccaagcttacgcgtaccaatcaaaaagaaccaaaattaaaaaaaaaagaaatgaaaaaaaaaaaatcttcaat

SEQ ID NO: 376 - DNA, 796 bp, Zea mays
ccggggttttcctagccactaatcagtctcctctcttgaccccgcgcacaagcaaatgtgaagcccccatccgccgatcggtcccgctgttcacctgcgcgcatgtattactagctaactgaccactactactgtcagttgtcattattgcctcgggcaagaaaccgacgaaccggtcggctgaacaagacggcgcgtgtggagccatgctttccagcgcgacgagagcagggacgtcgtcgacgaccaagcccgagcacgcggcgggcgccgtccacggagccggagccggcggcacccccatcgtcgtcaccgacggcgggcaggaggtcaccgtctctcagttcgtcgcgcagctagatgaggcggcggcggaggctggacagcatccaccagaggttgaggctgctggagcagcagatggagacgctggaggccgaggtaggcaaagccaccagcagcagagtgcacacctacgcctagtttgctagcagcggcggccttgccagatcgatccatcgacccgaagcagaccacagatctttgtttttcttgattaaatctttctcatgtacatgtacgacgatgatcacagccagggccggctctatgatttcaatggcctcttcccaactagaaaatataggccctatagactatttttctttttaaagtttatcgtataattaaagagataatattaataatgatatatgtatatatatattttttcgcaaaaactatgtctataatacaaactagtcctatgagcaaaaaaaaaaaaaaaaaagcggcc

SEQ ID NO: 377 - DNA, 988 bp, Zea mays
attccccgtcgaccccgcgtccggcctgtatatccatcgcacaagccctgcgtggtgcagcccggtctaatcagaaacccctgctctctcttcgcgttgagggtccagtttccttgtcctcgactccttccatgtctcccaaggacaagctcaagctcaaacggttggccccaggccaagtggagctctcctcacaggcgggggcaatccctgctatcaccgaagacgaggatgaaccacatacccctggattgtggcctcttttgtctctgaatggttcagatggcagcctgcaggggtttgaggagctattggcctcagtacttggaaagaaggcaggcgagaagggttccttcaagctactgaaggcacgggcctcggctcaaacctatgtcaagatggcattcacggtggagaagaagcttgctgaaggggatgtgaactggtcaaatattccagaatggaagacgaagcccaagaggatgagagcgcactacgaagtccttgctcgagtggaggagggtcaggcgatccctgagaggatagcgcaggtacagcctttccaggccaacgaggccatgtcggagagtgtgctcaccggcaacgtcacgaggtcgaagatggaaatcgtccatccaccgccggtttacttcactctctgagtgagggaagaactggatgctgtttgatgttctcttcagagctactacacgttgggtggtgggtagtttacgctggctcgtaggcaacaaagttgtacaagcggcaagagtttgttgcctatacactgactgcgttgacatgaatattgacgttctatgtgtactgaaaggctggcaagcctttaatattatcaattggaataatgaggaataagaaaattctgtacatctttagagggcaaaaaaaaaaaaaaaaaaaaaaaaaaaaaaaaagggcggccgctctagagtatccctcgaggggcccaagcttacgcgtacccagc

SEQ ID NO: 378 - DNA, 4311 bp, Zea mays
tttttttttttttttttttgcacgcgctgcccctgccatccgtagtagcggcactgcagcagggcagggcgagcgccatgtgcaaacacagtgcgccactcgagtcgacagcgaaatggcagggaaacttgtttatagatcactaaaacaagtactactactacgagtaataagagttcgttcattcgttcattacagatcactgtacacttgcttgcctcttttactcccatccttgggtgctgcctcaaatcaaatgcctcaacgacaacgaggaggagggagagcaagacgagaggtatggaggaacggaaccatgatttgaggatccttcactgcggtttcgccggaggcgacccgctggtctcgccgccgccgccgccgagaccagactccaagtcgccgtcttcattctgcggctgctgctgctgctgctgctgcgccggcgggggcgcggccgtggcgccgccgcagccaagcagtcgccgtagggcggcaacactggccgcctccacgaccgccgcgactccggcgatgggccctgctgctgcaaccggctgcgacggcgcggcgccggcgccccgggtgctgacggcgccgtcctgggagccgaagaagaggacgttggtggggaagttgggcgagtccgggtccgcctcgggcgcccgcggcgcggcgtcgtcgccggcgaccgcgcggcggcggcacagcgggcaggtggagtgggagcgcagccacatgtcgacgcactccacgtggaacgcgtggccgcagcggggcagcacccgcgcggcctcccccgcggccagctcgccgatgcacacggcgcagtccccgcccgccgccgccgcctcctccctcggcagcgccgcgatggcggcctcgtccagcccgccgtggcaccccgcgccgcccgcgaacacgaagcggggccgggccctgtcgccgccgatggtcggggcggccccgcggtaccgcttcgcgcacaggaagaggtagaagcagatgacgaaggtgatgaagattgccgccgccgccgccagcatcgtcgccgcggctgccaccgaggttcccgtcaccgccatcgggggctccggctccagatcccttgtcgtcctgttcctcgtcccaggaagcagcctgttccccagctttcttggctagtaccactgttcttggccgcggtggagaccggcggcgcgctctgagattgggagtagcgactagcttagctagtggctcgtgccgaattcggcacgagtaccgatccagccggcgagatggacatcgtggtgggtgcgctgtcgggcatagtggacgcgttgccggggaagctcggcgagctgctggagcaggagtatgcgctgctctccggcgtccgcggggatgtcatcttcctcaaggatgagctatcaagcatgcgcgccgccatccactactgcgagtccctcgaccaccacgactcccagaccactggatggatcagcctcgtccgcgaggtcgcctacgacatcgaggactgggttgacctcttcagcatccgcgtccacggcggtgcccaatccacttccgggtttcgtgcctggcttagccgcagcgtggacaagttaacggcgctccccgctcgccacaccatcgcgagcgagctccagggactcaaggagcgcgttcttgagattagccggcagcggaaccgctacaggctgggccaaatggttggcaccacctcgcaacatccccatgatcccagactttctgcgctcttcgtcgaccccggccgcctcgtcggcttcgatgggaaggtggaggacgtgtccaagactgtcatggacgccggaggtagtaatgggctgagaatcgtctccattgtcgggatggccggctcagggaagacgacgctcgcaaatgctgtgtaccggcgtctccaagcagacaacaccttccagtgctctgctttcgtctccatcggaccgaagccggacatggtgaagacagtcaaggatatgctctcaagactcggcgacggccaccgaggaggcgaggacattagccaactcatcccgagggtcagaggaatactggagaagaaaaggtacctcgcttggatcgatgatatatggagtagtgaacaatggggagtaataaggtgttgttttccagacaacagtcttggtagtaggataatcaccacatcaaggaatgatgcgttgcccaccaatcatcattacggttcaagcaaatttgtctacaagatcggcctccttactgacaatgaagccagggagttgtttctgaagaaagctttcagcagccggaatgactgtccacaacatctggtggatgcttttaccaaggttctgagaaggtgtgctggcttgccacttgctgtggttagcgtagctgccaagttagcacacaagcaatcaagagaagaatgggagaagcatggattgaacttgctatacagctcacattcagatgggtcagatgggctgaagcaaatactccatcttagctacagcgatctacagccacaactcaggtcatgtttgctgtacctgagcatatttcctgagaactcagaggttgagacagatcgcctagtgaggcgatggattgccgaaggactcatcgctgcaagcaatgaggatacggcaatcagttccctcaatgagctaattgggagaaacttggtacaaccgttggatctgaaccatgatagtatcccaaggtgttgcagagtccacccagtgatatatgatttcattgtttgcatgtcgatgcaagacaactttgccactgtaacggatgctcaacatgtcccaatcaacaacaaaactgtccgtcggctgtccctgaatttgaagagcaacagcaagcaagatcagcctgcagcacgaaatgagactactgatttgtctcatgctcgctcagtcactgtcttcggtcacgctagtgccacccctcatctgactgatctgaaagtggtgcgtgtgctggatctcgaaggctgcaaaggtcctgtgtgcttggatggcctatgcaagctggtgctgctgaggtacctgagcctcagaggcactgatgtcagtgaactgccagcggcaattggggatctaaggtgcttggagacacttgatgtgaggtcaacgaaggtggaagagcttcctcccagtattgtaaggctgcaaaagctaatgcatttgcttgctgggagtgccaagctgcctgacgggatggataagatgaaagccctgcggtcattgtcatgcgctgctaccacaaagagctctgccaacgtcgtggaagagctcagcaagcaccataacttgagacaactggaactgtattactatgctactgaaacgcctgggaatgagaagcaaatcaagttccccgctgatgggctccagactgtgaaacaactgtgcatccggtgcacatcaccatcggtgacatttgagccccgtgtactgccaaaggtccaagcacttgtcctgagattcgagaaaggccgtgctgatgacacgattggcgtgtctggcttagagcatctgtcgagcctcaagcatttggtacttgagtttgagcagcatgatgcgggtgccatggcgacagttactgcagtgaggatggctgctgaggggctccatctagatcatcaatatataactctaaaggtggatgggaaaaactgcggcgcctgcggccttctcgaatccctctgccgcctccaccgcctccggactccggtgaccggtgagtcggacaacggtgtggtcttcccctccctctgcagcggcgcagtgacgattcccctctgggcctcctgcctcccctcccccatcccacaacggtacaatggctatccggactccggcgtgcccgaaaacctaaccctaatccagatctggtctctcttcctcattcctccctgccctgcccttctctctcgtcggtgtgtcgtggacgactctggtgctccggtgcgcagcgcaggagccagagtcgccgatgcccagcgaggtggttggctggtcgccatggaacttgacgcggtagccgagcacgaggagcacttgttggaaacccataacgaggaagaagatgctgctcaagccctattcggtagtagctctgctgctctccgtcgggatgctgcagcggctccttccagcgatgttgggggaacgggctccagcagtgcgacccgtaccgaggcaagcacgggtaacgaggggcgaacgaggaagagggcctccagttctccagtctgggaggactttgaacaggtgttcaactttgagaatg

SEQ ID NO: 379 - DNA, 1864 bp, Zea mays
atatgaaaaatgatattggggcagctctgaggtttcagttgcagctagacggggctagtaacaatacatgagataagtgaaagggcctctagggtaatggttaaaacttccgagtaacccctctaggtcttcgattcgattccctcgaggacgaatttctaacatggttaaaaaaatccacttgttgggctttgcctgctcctgggttacatggggacagcctgctagtaatgggcactagggttcaaggatcgttttagttctatttttttttctcggcagaaccatgtttcagtctttttttaatataataccgggagaacagtttttccctttccgaccgagtttaacaatacacgagagatggactacaatgacattgcattctcgttttcctttgaccacactatatactagtctatgagaccacctgcaataattgacaacggcactgaattttgattacttattttctaataacatggaaaaatgcaacagcagaaacctgcgtgtatgtgtctgtatagaccttctcccccatgtacacacttctgcatttctgattctatagtaaactggccatccaacaatacacaccgatgtagagatcccttatggagcaccattcctgtcattagagcctctgttccagagaacctcagagtcaattgtgctcagatgtttatcatcaagcctcttccatgtcttgatggacttgacacctcgccattcaccgctgtctgttttctcaagttgcacagacagtaccctcgctcttcttgaccacccagctttcccataggcatggtaaccaaatccaccagcatagcagagctgaataccactgagcttcccacagaagtcattaatgtgatcatgtcccacaaaggcagctctcacatctccagcctccaccatggaggcaaagaacccagagttaattgatgctgagctgataccctcctgctttactcctgtgaaattggatgccgtaaaactgctgaactctggtagtggaatgtggaagaacacaaggccaggtgctggttccttctgtttcgggttcttattcatatactttgcctgtagacttgaagatgtttgttggaaccagacttgctgtgaagccttgatccaaccataaccattgattgacggcacagtggaatagtcaccgctgtccaggaaatacaggttgagcactgatttttcatccatggatgtcccttcaaccccggaaacttccaaattgtaatttccgtaaccatcaatttcaattccttccggattgaagcttgcaagggtgtttttcatgcccacaaggtgacgcatcacaccctcacgcgatagcgtaccctcttggtcatggttcccgatcacagcagcccaaggcagtttcatgtcaatggctggagcaattgctgcatccatagacttggctgcatcagttgaatcagcaccatagatgttatcacctgtgaagacgacgaggtcggggtcctcggcgcggaagacgcggtagaggaaggcggtggtgttgaggtcggtgcagccggccacctgggaagggagcacgtcctcgcaggccgtgctccggccgtccgcgtagtgcatgtccgccacctgcaccaccttgaacgtcccgctctcccgccggaaccgcagcttaccggacaccccctgcttctccttccccgaggcggcggcatcggccacggcgaacaggagaagcggcgggaggagcagcagcaggcaggcggtgcggcggccgcgtccgaggccgcagccgcccatgctgacgctggcgcagtgcggcgtgcagcgagaagagaatgaatctggggactgcctctgccacccgtgc

SEQ ID NO: 380 - DNA, 614 bp, Zea mays
ccacgcgtccgatcgaagcctggacgacgcgaccgcgcggcggcggctcgacgcaaccagcacggcggcggcgcacagccccgacgacgactctccctgctgccttctctcctgtgaggatctgctctcttccttccctctcctatcctctgctctcgctgcccccaaactctcgccctcgatctcgctcctctacgctctcgcacagtgacgctttgttcaaggtgtggccgccgccagtcgcctgcgtcgttgaagcagttgctgtcgaggttgaggtcctcctgggggagacgcgcggcgcggccgaggcgcagcgcggtcaggttcgggtatgacctgcatagctactcccagaacttcgacgatggcgttgcctagtcagcccggcggccacggctagcttcttcagtacgtgtagctagttaggagtctaggatattcgtgtaactagctacggccaggatattagtgtggctttaattagtgctttttgtcctgaccactgagatttcagtccagtgttaacttccggtgattatctgtgctctctagctggttcatccagttatattgtataaagaaattacttcatcttaaaaaaaaaaaaaaag

SEQ ID NO: 381 - DNA, 541 bp, Zea mays
gtcgacccagcgttttcggacgcgtgggtttgcttcccagacggccagatcaggcgaaaggcgtgacgggggccaccgctgcggcgccgcccctcgaccctccgctgctccgcctgcgctggagaccatccatctggaagagatctctgatttacgggatggacggagccaagccagtggcggccgggaagcaggacctggaagaggcgctgctccgtatcgtgcagcagcaccaccaccagtccctctgccaacgacaacaaaccggaaatcggagacatcgagaactggatgaagatcatggacttcgattgtaaaagtatcaatgcagccatacgcaatattcaccagtcatgactctgcccgcgattacaatttcacgatttttaggaagtatccgtgtcattacgttctctgattagaattattgggtctggaaaccctctttttatggaaccgtgtctcctgattttcccatgcaagcacctgtgctatgagtgtatgtacccttcaatttcaaagtgatgatatatataggatc

SEQ ID NO: 382 - DNA, 1738 bp, Zea mays
cctcgaggccggccgggccgggatttccaacgggaacacatctgtttagagctgtggctctgcgaggaatggagatggagtcgactcctctcttgacaccatacaagatgggcgatttaacctggcacacagggttgtctacaaatatatggctgcagctgaggtccggcaccacacaaacagcacatctgtttagccaagctgtggccaacctctgcgaggaatggagatggagtcgactcctctcttgacaccatacaagatgggcgatttcaacctggcacacagggttgtgctggcgccgctgacgaggtgccgggcgttcgggaacgtgccgcagccggagcacatggctctctactaccgccagcgggcgacgcccggcggcttcctcatcgccgaggcctgcgccgtgtcggagagcgcgcgcgggtacccggacgttccggggctgtggacccaccaacaggtcgaggcctggaagccgatcgtcgacgccgtccacgccagcggtgccgtcttcttcgcccagctctggcacaccggccgggcctccccctcaggttctctgccattggctttgtggttttctcattcatgttgaaatcggttttctttttcttttttggttgcatcttttcgcttgtgataaggactgatgaaactgggaagacaatgacagctgatatgtcagctacttcagagattttttttttctgttcgcttggctggcagcgcatcgtccggtcatcttgtggcccagtgccagtggccactgctggctacagcaattccaaccaaatgggcaggcaccgatctctagcacggacaagcaaatcccagcacaggtgaaccattccggtgacgtcaacacgttcgcggtgcctcggagactagagacagaggagataccacacgtcgtcaatgacttccgggttgccgccagaaacgccatcaaagcaggctttgatggagtggagatccacgcggcaaacgggtacctgttcgatcagttcatgaaggacagcaccaatgaccgagacgactcctacgggggcagcttggagaaccggtgccgtttcacagccgaggtgatggcagccgtcgcggacgaggtcggtgccaaccgcctcggtgtgcgcctctctccgttcgcggattatatggactgccacgactccaacccagaggcgctcgcgcttcatgtcatcgacgagacgctgaacccgctcggcgtgctttactgccacatggtagagccaaggatgcgggtgaacccagacgacggtattatgactctcgagcacatgctgctaccgttcaggagggcgtttcgcgggacgttcattgctaatggaggctacgaccgtgaggaaggcaatgacgccattgcccgtggccgtggctatgcagatctcatcgcatacggccggatattccttgccaacccggaccttccgatgcgttttgccaagaatgccccgctgaacaagtatgataggagcaccttctatacctctgatccggtggttgggtacacagattatccatttctcgatcaactctaggatgattggcttataaacaatttccccacctggagtactatcttgaccttaaaataaaatgagtgtcactataatattagtgtcgatcaaagttttctaagtttaactaaatctacaaaaattattattaagcatttgtcaaagtatctatacacttagtgattttgttc

SEQ ID NO: 383 - DNA, 1635 bp, Zea mays
aaagcaggctggtaccggtccggaattggggaatcatccgacccgagatcgctttccattcccatcgcttctcttctccacacgaactcactcttgagagatagagcgaaacacaaagcaggcaaaaaaaaaaggatagagagaaaaaatcattctcgtttctctcactccgctatacccccgcccgaatcgatggagattagggctccgccaacgagcctcagtctcgcgccgccgcccgcctccgccagcttccgccgaacaccgcttaggaccttcttccttaacggcagcgtttcacttaaagttgtgcaagtacgacaatcaaatgtcaacagatttaagtgcaatagcatacaaagtaacctctttgatagactaacgagggttgccaggtcctacgcaaatgcagttctaagttcatttgaggaccctgagaagattctcgagcaggcagttctcgagatgaacgatgacttgacaaagatgaggcaggccacagcacaggtcttggcgtctcaaaagcggcttgagaataaatacaaagctgcagaacaagcttctgctgattggtatcgaagggctcaacttgctcttcaaaagggtgacgaggatcttgctcgcgaagcccttaagcggcgtaaatcatatgctgacaatgcaagctccttaaggtcccagcttgatcagcagaagggtgtagttgaaaatctgatttcaaatacccgggttcttgagagcaagatagcagaggccaagcagaaaaaggatacgcttaaagctcgtgctcaatcagcaaaacaaatgtggatcttgtttcacttcaggactgccacaaaagtgagcgaaatgttgggcaacgtgaatacaagtagcgccctttctgcatttgagaaaatggaggaaaaagttatggcgatggaatcccaagcggaggcactcggccaattagcaactgacgatctagaaggaaagtttgcactgctcgagaattcatctgtcgatgatgatctttcacagctgaaaaaagaactctcgggaagctctttgaaaggggagctcccatcaggcagaagagcggccagcaactcaggcgccgggcgccctttccccgacctagagattgagagcgagctaaacgagctgcggaggaaggccaaagaatactagagagtagagactctccaccaaccatccttcccgggcctgacagaatctctcatttccaagtacatatagcacagctgcagatttgctccatcgcgccttggaaagttggaacagaggtgcgcctttgactgtcactgtcaaagggtatatggagtcgccgagtttctcggcattttcccgtcgacggacatttgtacagctagccataaattgttgtaaatcactacgctccctagagcccttttttttgcgtactacgtatatagtcaaagtttgaagtttccgcgtcgtggtttttacccccattttttccgtgccataacatgtgttttgtgactggagacttgggatcctcagccaggtcgatcaaatactccctttttttcttcttcttataaaaaaaaaaaaaaaaaaaaagggcggccgctctagagtatccctcgaggggcccagagcttacgcgta

SEQ ID NO: 384 - DNA, 1518 bp, Zea mays
tatttagtgtgaactatagaacaagtttgtacaaaaaagcaggctggtaccggtccggaattcccccacgcgtccgcccacgcctagaaaaaacccgcgacaaccactgagccgagcgggggtggatatggcagcggtcgccgcccaccaccacctccacccctcctccgtcgctgcccgaactccaacacttggccgacgccaccgtttctcgtcgcttcctctcgccgcgcggccgtcctcccaaggccgtttcagctcccgagccgcgacccgcgtcctcacgcgcgccggaagcactggcgccagcagcgccgcctcgcccgccgcgacgacggcgtcgtcgttgtcgctggaggagctgcgcgggggctgcaccacctggacgtggcgagggatgcgcgtgaactacctcgccagaggacaggggccgcccgtactgctcgtccacggcttcggcgcctccgtcgcgcactggcgcaggaacattggggtgttgtctgaatcctataccgtctacgcgatcgatttgctggggtttggcgcgtcggacaagcctcctgggttttcttacacgatggagacatgggccgagctgatactggatttcttggaggaggtggtcaggaggcccacggtgcttgtcgggaactcggttggaagcctcgcgtgcgtcattgctgcatcagagtccagcagagaagccgtccgggggcttgtcctgctgaactgcgccggtggcatgaacaacaaggcgatcgtcgatgactggcggatcaagctgctcctgccactgctctggctgatcgacttcctgctgaaacagcggccgatagcatcagcgctctttaaccgcgtgaaaaacagggacaatctgaaggacatcttgctctctgtttatgggaacaaagatgctgtggacgatgaattggttgagatcatacgcggaccggctgacacggagggcgccctggacgcgttcgtgtcgacggtgaccggcccgccggggccaagcccgatagccctgatgccgcggctggccgacctgcccgtgctggtgctgtggggtgaccgggaccccttcaccccgatcgacgggcccgtggggaagttcttctcgaagctgccgtcggagctcccgaacgtgacgctctacatgctggagggcgtggggcactgcccgcacgacgaccggccggacctcgtccacgacaggctgctgccgtggcttgaagcgctcctgccgcccgccgccggcgcggtgactacgacgacgacgacggttgtctaaggctaataataatacgccttattgctgactggatctttgaagtctagttggactcttcatttttaactctctagttatctctttataaaaaataaattctataaatcttatagattttgtttagaaccaactcagtttttaaaaaattggcttttattttttagctagcagtggccaacttcttagtttctaagagcatctctaatctaacatagttcctatttcac

SEQ ID NO: 385 - DNA, 798 bp, Zea mays
atgacgagcgcctccgagctcttcatcgcgcgccgcgccgcccgtggggcccgcctctccgaccccgaccccgacccggacccgcaccccgtcgcgctccgggaccagcatggactcggcggccggcgccagcgccgcgggtgccggccgcgacgccagctcgacgccgccggggacccaactaattattgggcacgactaatatgcaagcaggatcagatcttatcctacatagacaataacaactttggggattctgcatctcggaggaacagactcgataggctgatgttcaggacaaatgagcggcttcctggtgctgtactgcaagcacaagcacgtgtcctggagagactcagaggcatttctcttggttcatctgcctccaggccatccatctcattagatgaattttccgctaacgatgtatttagaatcatcgacttcagaaacagggaaacgtggcatgaggcaaaccggccccactcatcgtcgttccacctaggcagtgaatcagatgaagaaagaccaaacgtcagctccgttaactccaaccggtcaactggacttagcaaggcggctttcctgcgtctgcagattgagatttttgaggctagcaaagatgataacagagaggcttcaccagaatgctctatttgccttgatggattttacgacggagatgagctgataaggttgcactgtggccacaggttccactcgacatgcttggagccatgggtgaggaaatgttcagactgcccatactgtcggacaaatatacggtcacaatcctga

SEQ ID NO: 386 - DNA, 1027 bp, Zea mays
cgaccgcaccgtgtgagttcaaaaaacttacccccaaaaactgtcctcccttggttccttcccgctctccagccaaagtcgccgctcccgctcggacacgcgtctagaagcttctggatccgtccctgacgccgatggagcagctcgactacaagttccacgagaagtacactgctctgaagaagcggaagctactggacgaggggctggagcggaagcgggaggcggaactcaatgagctttacgatgccatgaaagattgggtcagcgaactcaggaaagacaatgcggaactcaacgagaagctggtggagaagcaggatgagctcgagaaggcccgtcaggaattccttgaagatattcgtacaagggacgctgaaattctgaagctgaagcagctcttagatgagaaggctgaaaaaaacaattctgcagctactgggtttccttgcctagcgcctgaagctgtcctagaaaattcaactccattgtcacctaaaagaaagacaccattttcccacggcaagcagctagaatgctccagaagacatacacgcatctcagggaataggacaacagagattcggagtgctcatatgctttctttgctgcttcaatctttagttcgcatgaagatctcggtaaatgatggaacagaaatattttcggtttcagtatctcatgaagctagtggttacagtttcatcctcacctggctagaaacatctgatgaatggtcatataagctctcctcgttgggcacactggaaaggatagccgttgattggatgaggcaagacataagattcagcatgaaaatgtgccgcatgttctttgagcggatatcaagcatcataacaaaaggatgacgacatgctcttcgcttcagctagttatatcagtctgcctactgtacatggaagcatcctgatgtaaaaatgatttgatgcactatcttattgcagagctgtctttcatgctacttgcgcccgtatccctgtggtgtgcttgggtgccggacaaaaac

SEQ ID NO: 387 - DNA, 722 bp, Zea mays
aaccggttctccactcgtccatcgcgcgccgcgctcgcaaccacccacagtgttctcgtcatcccatccctggctagcagataaagctcgtaaattttggatcctaaaacagtcgtgccgatcctccctataaaggcataaagcctagctcccttccggcgacgatgaagcggtcgaggagcggacgcgagagggcagaggagatattgaggtggaaggctgcgtagagcgaggcggcgacatctgcagcgataccggaagatgacgctattcgagcagctgttgaggagaccgttgtcacctcctcgtttgagcccgaggcggccaccagcgctgatttaggcgtgtcgctggtgggcgacgacgagcgcacggacatgataccgctcaagttcctccgtgcgcacaggaacaaggtgaaggaggccatggcgatgctcaggtcagtggtgttgtggcgcaagcgctttggcaaccatgagctctaggatgtcgacatcggcctaccggagctggagcaacgtgatgttcctccacgtggttggctgacgctcgctgctcctcttccttcctcttccccatgaccgaccggtgcccacgacaaccttcctccgcacgttgtcgctccgtgtgacgcccacgttctcttgcgtcacgtccactccctgattatgataacataatttctcctgctcaaaaaaaaaaaaaaaaaatttgcggcc

SEQ ID NO: 388 - DNA, 1116 bp, Zea mays
atgcggtatcacgtgaaggtcctgtccccgctcctcccgcccccggcggaggagggggcgctgacgacggaggctgcctatgccgccggcgacgaagacgacgaccaacctgatgctgatgggtcggcggcgttggcaagtcatcgaagagcagtcctattcaagcatagggctctggacgactacttccaggggaagctagtcgagtggacggagcaagtgatggagagaggcgacgtcatgcactccgccgccgtcgtggcgccggaggcagaccaggaggtcatcgctctcgcacgcagggccttggcccaggggcatcgcagcgtcttggtggacattccgcaggtgcacatggatttcgagccgctgcggccccaggagatcctctactacatcctgtgccagctccagcaggatcgagcgtcatcgccgccgcaactcgggtcgttagaaggaagcccagacacattggtgaagcagtatctcctctacagaagcaaacgggagatcagccatggaaaagagagtctactcggccaaactgagatccagcaggacattcagaatgtgatgggcaagcttgacgaggtgaagaggggcattcaagatgagcaacttctgttgcagctgctggaagggatcagggacgtcaaggagctggaagagaagcccatatccgtgctcttcgagctgctggtgctgcaacagcaagctgcccggcgagaccaagcgaggggcaaagccatgcgcaaactggcagcatggcacgcccacatcctccggataacagcagagagtctcaagaagtacatagaggcagagcatctgccgcaacaggaggcggagaagaagacggccaagaagtttgatttctatgatgcaagatatggatatgtcatacacaaggtgtttgcaacgaaggccagcaacagcaaaaggcgccatgggcatgtcaagatccaaggcatgatcagcaaagtcccacaagatgttgccaccgaaaccgaggagaagatcgatgagatcaagaagacggtccaagagcagatggaaacggggtggatcgtacgcaagatcatcgagaatcacttcgaggacgacaagaggcctctg

SEQ ID NO: 389 - DNA, 974 bp, Zea mays
acgtgaaaaacaaaattatacactgttgcattatttggtgggcttagacactattgtacatcagtgggatatggaatgtgcatattagaattgttatagtttgttagttgcgtagttaatacatgtatgttttcagttatatatattagacctttataagaaacacaactctgtatatttgcaagtatggaacgtggccgacaatattattttacaccaggaggtgccttagcagcacaggtggcgccttagtctggttcctgttctagtaaggctgcatacaatgcattcttctttgtgtccatcaagtttgtgccttggaggagaatttggagatcctcggcaaccctggggtgtaaattttttttgtgtcttgctgtcaaaaagtggtctggatggctaatcaaaattgtacaaacaatattgtacaaacatagtttaacgcatcctgctgctagcttattgtgtgatcaagctgaagagaccatcctgcatattttagtttcttgtattttcgctcggcaggtctggaccttggtattacagaagcttggacttgtggcactagcacctgattctgatgtaacaagtttccccagctggtggtgtggtgctatcaagaagatacctaaggacgtgaggaaatgatttaacacttaaattatcctggtagcttggaaaatatggaagcatcgaaatgcatgtgttagctcaaacgtgcaaatggttttacaggcggtgggtactgagagaatcctttggtgctctccaggagcttctgctctttaggagctgctcccccccccccccttcagattaggtttgagttttcctggctgtggttatttgattttttttaggtgtgggtgtgtgagtctcaaggttttcctgggagaaggttccatgccctctccccctggtgttctttcttccatcttaatgaaatgacatgcagctcttctgcatggttcgag

SEQ ID NO: 390 - DNA, 820 bp, Zea mays
actatagaacaagtttgtacaaaaaagcaggctggtaccggtccggaattcccgggataccaagcaagcacaccgctaagcctgctgccgcctcctcgcggcccagcagcatcagctaactaacccgcccgcgcgccgtacgtgcgcgcgcctgcagctgctcacaagtcacagctagatagtagctactagctaggcagctaggaagagaagacgatcagcatggcgggggagatcgtggcaccgtcggggagccggcgcagctggctgtcggcggcgtccatctcgcggtcgctgcacgcggcaggcgacccggacgacccgttccggcggtcccaggctagccgcgacgacggggacgacgaggagaacctgcggtgggcggcgctggagaagctgccgacgtacgaccgcatgcggcggggcatcatccgcagggcgctggacgagggcggcgccaaggcggggcgcggacgaggtggacatcgcgaacctggacccgcgcgccgggcgggagctcatggagcgcgtcttcaaggccgtggaggacgacaacgagcgcctgatgcgccgcttcagggaccggctagacctggtgggcatcgagcttccccagatcgaggttcggtacgagcacctgtcggtggaggcggacgtgtatgtgggcgcgcgcgcgctgcccacgctgctcaactccgccatcaacgtcgtcgagatcgatgcttgtccattccttcccgctcctgtggacagtgacaagcgggagcgggagtacaactattcatttagggggtgtttggttgcccctgctaaagtttag

SEQ ID NO: 391 - DNA, 231 bp, Zea mays
atggatgcggcgtcgatgctggagctgtatcgccgcgaccggcgcaggctcctcagcttcctgctctccgccggcggtggaggcggccgggccctcgatctctcccgcgtcgacctcgacgccgtcagcgccgactacgcgctcgagtgcgtcgcctccggcgcccattttaatgcttcggaagccacgaggaggtacttcgacgagaggaggtaccctatcatgctgtaa

SEQ ID NO: 392 - DNA, 555 bp, Zea mays
cccacgcgtccgggccgcccttttttttttttttttgtacatagcgtttacacttgaattgaaccaaacaatatatcggatcgcagcgtttacacttgaattgaaccaaacaatatacgcagggtcgcaggcagcggctgaccagctcgctagcggtggcggctggctgttggagaacgcagccacacagggtcgcagcctcgctcctcgcagggacgccggacctagaccgctggacgacagccggacctggacggcagtcggcaggaacgcaccacgggtccacggcagccggacctggaccacggcagcctcgcagggacgtagccacgcaggaacgccgaacgcaccacgggccacggcaggaatgcaccacggcagccggacctggtttggcctttctggccgccggcggctggcggatggggtggcgcggctgcttgtggctggagcgctgggcgtgcaggcggctggcggatgagaggattgagagcgagagtgcgagacagagagtcagagagagacacggcgtgagagtctggttctagatcgcga

SEQ ID NO: 393 - DNA, 1127 bp, Zea mays
agtaaaccactccttgcacacgaaacaaatatatgagaccctctcaaaggatgaatagttgcttgccttaaattagtacatcttataagaaccttgtacgtcattaatgcatcgaatgctctgaagtcttaactctgttaatactttactgcttattacacatgcagaagaagaaaaaagaaaggtagagaaataaattacacagagagagaatagtgtggctaacctcattttgtatattggtcatctgaataaacatgtactgcattgttcctagtatctcttagtttacagtagtttcacatttgtgtctctttttgtaatattttgctcaaaatggcggaaacacacaactttctatgtatcttgttgcattgcagggctggttattcacgatggggagccgagaacgttctaaatggtcgaactggtgaaagaatggaatccttggttttcatgggccctaccttttaccagaggctgattcatatgtccgaagataaggtgaaataccgtaatactgggccggtccaccccctcacgaggcagcctgtcgctgacaggaaaaggttcggtggagtcaagtttggggaaatggagcgtgattgcctcctcgcccatgggtcagctgccaacctgcatgagcgcctcttcatgctgagtgacttctcccagatgcacatctgccagacgtgcgagcgcgtggctaatgttgtcatgaggtctgtccccggaggcaaaaagatccgtggcccctactgtgggttctgcaagtcttcggagaacattgtgagaatcaacgtgccctacggcgcgaagctgctctaccaggaactattcagcatggggatctgcctcaagtttgagaccgaagtctgctagagcttgtaggcctgcttgtatccagtcatccagttattatgtacatagagttagcctatgacgatctcatgctggcagctatgtgaaattgtgtgccaatgtgtgatcccggcggcacagcaatgatgggcgagttcgatggagtgaaacggcagtctacctctgcttagagaataaactctgttgaaactagaacccttgcagcgtttatatgctgttttcgtcctgcccatctgataaactat

SEQ ID NO: 394 - DNA, 773 bp, Zea mays
ccagtgcccgtctctctgcctcccttcctttcgcctccccactcggcgggtacgggacctgggcggcgacttcaacgtcgtccgcgggaaggttgagacggcgggggctggaggtggtgtgcgaggccacgacagggcggcagccggactcggtcaagaagagggagcgccagaacgacaagcaccgcatccgcaatcacgcgcgcaaggccgagatgcgcaccaggatgaaaaaggtcttaagagctcttgaaaagcttaggaagaaacctgatgcccaacctgaagaaataatcgaaatagagaaattgatctctgaggcatacaaagccatcgacaagacagtgaaggttggtgccatgcataggaacaccgggaaccatcgtaagtctcgactggcgaggaggaagaaggccatcgagatactccgtggttggtatgttccaaatgctgagcctgtcgccgccacctaggattcttgagaaatcactatcatggattcgtggcagcttttcatcaaatttcaccaattttgggcaacatatgctattcaaggatctcgacagcttgccaagttctctattgaaatggtctgttcataccttttctgctatcttctatgtctatgcgcttgctctgtattagcagttaaactactgactagagtggccacctcttctccccccgtttctgttgttgttcacttcattgttgtaaaactaaagtcagaaaaatttaaatatagctgttctctggtgtgtccagatttcttc

SEQ ID NO: 395 - DNA, 282 bp, Zea mays
atccagtgttctgtcctagcgccgccgccgccccagctcgccttgcgtgactgagagctcgtcggcttccgtccacgcgtgaagcgagagcatggacacccaggtgaagcttgctgttgtggtgaaggtgatgggcaggaccggctccaggggtcaggtgacccaggagccggtccggcccatcaccgtcacctgacaggcgagcggggttttccacggcatgttgtaaagtttgtacattctggttgccatgaaaatctatgtaaaattcaatccaaaaaa

SEQ ID NO: 396 - DNA, 557 bp, Zea mays
ctgcagctatgaagaccctgacaactttctacggctaccatccattggagatggtaatgcacccgctgggactcttccccactgagaagagggatgaccccatgtggtgcaaccgagtgagccacgtgaaggacatgtgggccatgcctcccgaccaagttgggaggatcactgttcagtgcatgagcacactgtaccgccttcaggccctgcaaagctacaccgtggcacacctcactaaccttgctcagaccactaagctcaccctcgacagccgagaggacttcgtggtcgacttgtcttcagaattgctctatgatgtgcaagaggagcttgaggaagctaacgaccacctggagatgcaccaccaggatatggaggcaaacgaggccgaaagtgagggagaggaggctcccgagaaattggaaccagcccctgattcagatgagaagaaaagggactgtttccaaagagggctcaacaccaagcttcgtgagtgcctcaacactgttcatgccgacaacttcaatgaactggtcaaccttgccatttcatag

SEQ ID NO: 397 - DNA, 656 bp, Zea mays
aaaaggacaagcagaccctcctccggtcctcccactcttccctgtccctctgccagctcaaccctctaccatggactaggaggccctagccttcgccgcgtgcatgtctgctcgggtgcagggacataggcatggaaccgcccgcccccgcagcgccacgaggggccgagcgcgccgaggctgtcgctgcggagccggcttccggggccggggccagagtcgaggtgcaacagacggggcccgagggctccgtgaaggcgagccgcggggggcgggaggtgccgctgcggctgcgcctcgggagggcccgtcggcgggcgggacctgtcacaccgacgccgtcgtggaagatggaggacgacggagtccgggaggggccgtccgcgcttgcgcctgcggccgcttccgccgcggcgaggaggagctcggcctcggcgcgccagctcggcgccagtctctgggagatccacgacgtggcgcgggaggtacgccggagcaggcgacggggtgggagggccatcgccgccggtcgggagcacggctgcggcggagaactggaccagttcattgatttgcttgctggtaccgcttttgaccaccccacccaatgcagttttctcaaggtccaaagtactgtggaccttcagttggtgtaa

SEQ ID NO: 398 - DNA, 817 bp, Zea mays
aattcggcacgagattatgtatggcatgcagtcagggtagcgatgctttttcacagtctggcctgtattcaaatcaccaagacattggaccacgttatgcgggggtacttctcggattgtcaaacactgctggcgtgcttgcaggagtttttggtactgcagccactggctacattcttcagaaaggttcttgggatagtgtgtttaaggtgtctgttgtgctgtacatagtaggaacggtggtgtggaatgtctttgcaactggagagaaagttctcgaataagctgtggacattcctatatgccaatatacgaaaattgtatgattttggtatacaaaggtaaatttaagaagagatacagctggaagaaaactccattttgtgcaagagatataccttcatctaatgtttgggctatcggaagaatgaaatcaaattagagtatcgtgtgaggtcaagaaggtactcatggaactggagaaacttcttgtacagaaagcaactgcaccaatggcacgtcagattttgcagctgtttcttgagaagtacaggcttctaccaagatcatcaatccacatatttttgttttgtttttaaaatacatggtagttaaatcgtgtttcaacttgcccttgattaaggcgctacagctactgccaactaaatgtagtattatgtgagctctttttcatcgagaattcctaattgtacctgcttcagataccttaaacgtctctgttccttgcgtcgttgctattatcgttttgtgtatgctgcataatgatattgcatggtgatggtgtcatttttttggc

SEQ ID NO: 399 - DNA, 727 bp, Zea mays
ttgacgcgtgggctggccgacagcggcgagccggcgagtggcgacaggaacacgcgagggcgcccaggccccatgcgcgctgagccgctgacccgctgtgtgtctgctactccgctgcggcgtgcggcgtcccgcaaggccacaagcaaggcagcaccgctgttgctggcatcttggcgttggcgattaggcaattggcatcttcatcttgctgctgctcggtcttgctgctacagtttcttggtgttgatttggcaatttcttgctctgctcgagtgctcggtctcacggtctgcaactgcaagggccaagggccaacggcaataccgcgtacagtgactaaaatatgaagaagcagagagatattgcctctctttttcgcaatcatgctgcaaagaaacagaagtttattgcgtcgagacctcaagaacaagaggaagagaggataaataaagaaattgtgaatccaatgccatcttcaccgcctccgacaacattttatgatgtcagtcgtcttccacatgatccaggtgaaagacaacctatggcaggtaccatgctaacgatgcaattcgagagcatatattttgaaggagccggttaaccatatgcacatgaattaccaaaaggaaaattggaatagaagatcgccattttaatgtcgagtgaatgaagaagacatatgtgaactgtatggcgttgaggtagcgcagccagataagtagta

SEQ ID NO: 400 - DNA, 685 bp, Zea mays
actccaagatgtggctgggtatggagtggctttggcagtaaaagatatctacgctgatgcctttggagaaagaaatttgggctctaaccttgtgggcctgatggtaaaggatggacgacaaggaaaggtgaatgccaaaggttactacatttatgagaagggtgcgaagccaaagccagaccctagtgttaagcatgtgatcgaggagtaccgaaagcaggcaaacacaatgcctggtggaaagcctgttactttaatggatcaagatattttggagatgattttcttcccagttgtgaatgaggcatgcagggttatggatgaaaatgttgtaattcgagcttccgatcttgatattgcttctgttcttggaatgggctttcccaaatacaggggtggtcttgtcttctgggctgacactgttggagcaccttacatacattcgaagctaagcaagtgggccgaaatgtatggccccttcttcaaaccatcatcatatttggaacaacgagctaaaagtggtgtaccattggccacaatggagcaggctgttcgtcatgctaaccattttgttattctccgcagagcgcaccaggaacatcacagcaaggttcggcgaggtcgcgcatgtgaggtgctgtccagcattgggaagaagaattcagaatacttgcggccgggatga

SEQ ID NO: 401 - DNA, 1610 bp, Zea mays
cttcgctggatggaggacatggctttgttagtcaacaaaataaaatgttctatatctccaatgcccccaatatactcatcagcactgtgagtgtcaatctcatttttgcgattgcccattatatacgattggaatgggaaaatactaaactggcgcctttctaccccccaggatgcagctcgtgaagagcatgctaaggaaaagttcagaccatatgcctactgtaagtctaaaaaagatcagttaactgttccggtcaaggcctctacggtgccgacacgatgttaacattccctaaaaaaatttacaggctgcggagctgctgcccacgcgccaccccacgcgttaccgtccgcgtgggttctaccccccaggaagcagatcgtgaagagcatgctaaggaaaaatccagaacataggcctactgctggggagctgctgaggcatccacatctgcaaccatatctttctgaatcgtccaactgttcaccgatctatcttccggtgaaacccaccaaaggtaacctggtagacaagcagtcaaagaagccaagcagtggcagaaagcgagctgtcaaagccaatggatccaatggaacattagaaactgcagcagagcacgctgtggaagcaagagacagctccacaaacttctcagatgtatcaactataggtacccaggaagccttgatattgtaaatgccggtggatcctgatgccggaagcaaagaagagcagaacgttgatgttgtaccgtttcagcatgcggcagagaatctgatggcgacaaccgatagacaggtcgatgcgaccatacgtcttaaagccataagaaccagtaacgcaaaagaggcggccccggtcacagttgcagaccaaaagttcaacgaggcgccaataccaaatgaggaactgacaatcggggtagtgcaggaacaaaggaaggacgtgaagccacgctcttaccaggcaggaaaaccagagatgtgtgacacaaccgtaacggaggaatcgtcgcccatcagcacgctaaagctggcgcacacggagagcgcgcccgcggagtgggaccacctgaacatagtccagcagagggccgacgcgctggagtcgctcctggagctctgcgcgaagctcctggagcaggagaggctcgacgagctcgcgggcgttctccggccgttcggcgagggcgccgtgtcgtttagggagacggcgatctggctgaccaagagcctgatgacccccccacccaagttcggagagtccccgacgaagcttttgtaaaacagtgattcgtttgagagtttttgtttttttttcgagtgccattttcgagagtgtacatagcatcaacagaatttgagttcaagattgctgtgcttacctaggcctgagctttatttatcttggatgtatatggtttgcatggcaccgactgttgtgagcgatgactggttatttaccagccgccttgttttatttattgtgctggaaatcatgccgagtccatttctgtttaaagagtaataaaaaaaaaataaaaaaaaaaaggggtccatttctagtgtatccctcgaggggcccaagcttacgcgtacccagctttc

SEQ ID NO: 402 - DNA, 659 bp, Zea mays
cccctgtcactgttgcagaccaaaagttcaacgaggcgccaataccaaatgaggaactgacaatcggggtagtgcaggaacaaaggaaggacgtgaagccacgctcttaccaggcaggaaaaccagagatgtgtgacacaaccgtaacggaggaatcgtcgcccatcagcacgctaaagctggcgcacacggagagcgcgcccgcggagtgggaccacctgaacatagtccagcagagggccgacgcgctggagtcgctcctggagctctgcgcgaagctcctggagcaggagaggctcgacgagctcgcgggcgttctccggccgttcggcgagggcgcggtgtcgtccagagagacggcgatctggctgaccaagagcctgatgaccccacccaagttcggagagtccccgacgaagcttctgtaataacagtgattcgtttgagagtttttttttcgagtgccattttcgagagtgtacatagcttcaacagaatttgagttcaaggttgctgtgcttacctaggcctgagctttatttatcttggatgtatatggttttgcatgccaccaactgttgtgagcgatggctggttatttaccagccgccttgttttatttattgtgcaattgtagcgtgagcgtaatggctgctgct

SEQ ID NO: 403 - DNA, 1937 bp, Zea mays
ctatagaacaagtttgtacaaaaaagcaggctggtaccggtccggaattcccggcatggccactggcctggccacagggctctctctctctctctctctctcaaacaagcttggattcctgacagtgcaagcaaagcaaagcaagcagagccgaagcttggagtatttctcttgcttcatcccctctcttatttacacaacacatctgagagacatacagcaagctctctccccattggccatatctagctcgtagtctacactgcctctaaaccttgacatttcctttgtattcccctttccaatttgcagtagtttgtaggtagaccgcaaggtggttttttcttgcagcaatggcggccaaggtttgacaaggcatgacgtcctgattaagccccctcgcggcagccacaagatttgttgcagcgtgagacaggagctgcggctgcggaggaagtgagcaaaagcatggatctttccggcacgcagggcgagctgccgatggcaacaatgcacgccggcgggtcgccgtacctggggctgcaccacgggcacccacagcagcacaaccatggcgcgaacggccggcacatgtcgccgccggatgtggtggcggaggaggccaagaaccggcagctggcggtggtgcccgtgggcacaggaggcggaggcggggcaggggtgaggtaccgcgagtgcctgaagaaccacgcggctgccatcggcgggagcgcgacggacgggtgcggcgagttcatgcccgcgggcgaggaggggtcgctggacgcgctccggtgctcggcgtgcgggtgccaccgcaacttccaccgcaaggagccgccgggcggcgacgggcggcagctgtacgggcaccaccaccaccacccgctgagcccgctggccgcggtgcacccccaccatcaccgggggctgctggtggcggcgcttcccccggcgccgacgcgcatggtgatgccgctgagcgccgccatgcaccatcaccagcagccgccgcagcagcaccacagctcggcgtcagcggactccgacgacgcacacgtgccaggggtgcgcgggggggaggtgcagcagcagcagcaggcaccggcacggaagcggttccggaccaagttcaccgcggagcagaaggcccggatgctggggttcgcggaggacgccgggtggcggctgcagaagctggacgaggcggcggtgcagcgcttctgccaggaggtcggcgtgaagcggcgcgtgctcaaggtgtggatgcacaacaacaagcacaccctggcgcggcgcggccacgccgggctccccggcgacccggagctgctggggccgcacgacggccagctcggcatgccgctcccggagccggcggcggagcccgcagctgctgctgcggctgcggctggacttggagtgaccgaccaagccatgtctttcgggactggacgacattgatcaagagagtctaatgctgactgaatcagccttgcggcatagggaagctgttttctttgttaatctgctgctgatctcatcaatctagccgtagctttagctgcattcgcattccactgttccatccatccatcctagctatccatcaaccacaaactgatgatgcggagtctccatggatttctattccttgttgatttctgtagctgctctccccgctcttgactttaattttttagtgctcgagtgactggtggtttgggaggtgagactgaatcactcactcttccgccttatgtgttgttcctttcatttcatcatgctactgtcgttgtggtgtttagtcagccagccagagagtgagtgctacactagtactagtgattgatcaacagctggtaatctgatacgcctggctgccgtgcatgcttagcaggcctgtaaccgaaagaatctactagtaaattatgatc

SEQ ID NO: 404 - DNA, 1314 bp, Zea mays
ccacgcgtccgccgacacgcacggcacgagcacgacagatcgggcgactggagtcatcgccgccgggccggtcgcggtcggatccccccgctccgcctcctctccttcgattcgttgcttcccgcttcaaatccaagccaccaccctttccttcccatggcttccgacgccccggcggagcaaccggcgacgcagcagaagcccaccagggtctcgctgtcctatgaggagatctccaagctcttctccctccccatcgctgaggcggcctccatcctcggagtctgcaccagtgtcttgaagaggatctgccgcacccacgggattgttaggtggccatatcgtaagcttgtttctgggaaagctggggatgacacaaaaggtcctgatagcgacaaagccaacgaactccttgaggtatcaaaaatcgcgaaacaaaaggctcccagtgcatcaggcccatcagtagtgtcatcaagcacttcccaaggagcggcaaagtctcaacagggcaattctaaggcaggacagttttcagtttcgccaccaacaggtaaacataacgcatcactaagtttgacccatagccaagccaaggccatcccttgctatatggatgatttcaagtacggattcccgtcatctggcttgtcttgtgaaactatgaaatggtggggcacaagcagcgacacagactatgtgcctaccaaagatggaagccatgaaccccatgaatcaacaacacatgagccatcaaagggcatgactgatgatgacgagttggactggggagcagatgaagcggaagctgaagctgatggcactgttacggcagaggcatcggcacagctgtgctcgctgagaaggaaagcagtagatgacgggcgcaaactattgaatggtcacaaccgcaggggccaagaattctctaggctgaacaaaaggcagaagacagcgctagcccaggtatttggggcttcactgccagaatgttgtattactcgtgtctagtgagtgaagaacaactgtagctgtgttttccctccttctccagaccctcatgtaatctgacccgttattagcgagaagtgtgaaatcttctcactgaattgaagaatggcaggagcttgttttgtcttccttatcaacaagttgttgctgccgcttttgtctacgtattttctctcatctcccatgtgtgtacgaggagagcaccttatgttatatctgtgaatgcaggtatatatgtacataacagctgtgtttcttctttgttttcgataacattgcattttgttctttgattcccaaaaa

SEQ ID NO: 405 - DNA, 2287 bp, Zea mays
gtgcaaatcttcttatagctccatgtcatatcagatacacttgggttactatagcttaagtcaataaaatataagagcagtagcagtttacttgtgtatcttcaaacaatgagaaacatgcattttccacacactctgtctctctcttaaaaaaaatccactaactacaactacagctacaaccactaattctgcagccataaacatctcactgaacatagcttttatttgcgactaatatttttctatgataatgaacaataaactaatacaattcggacaatgatgagcccaacacaagaatattgtacaattccaacaatagaaacaattgcaatcatcattcacgtatagtatgtacccaaacctttaggtgcacttgccgatgccacctagcaaaaaccgccgcataaacaaactcttccgagctatagcgcgcacctacataacgtgctcgccttccgcttcctccggccgatacatacgccaacgccgatgccgacgacgacgagcgcatcgacgacgagggcggcgcagcgccgccggagaagctgcaagggtccaccgccacgccagccatggtgctgctccttaggcctcgacccctccacggtcgccgccaccgccaccgctgccggccggagccccctccccgcaccgccgcaccaggtcgcgccgccgctctcccgccgcatccgctccccgggacgcgtatcccccatcgacgacccgaccttgcctgcctccgtgggttcgtctgtttccgtgcgactgtcgtccgtcaccgaatgcccgcctcctgccctgctgggtccgccgccgggcgctgtggagaagccgagggaaattctgaacctgaggctggtcgacaaaggcgtgatcttggaggtgaatgagttggagagggtgcgcggggagagcgaggtggtgaggaaggttgttggggaactaggcggcgagttgacggtggaagggatggtggaggtggaggccttcagggaagcggtggacatgatgttggaagacgaagacgaggccgctgcgatgaggcggctcgctcgtggtggtgtcgcgcgagccatcgacgtgctcgaggtgagctctccgctgttatattacgctgctactgcttgcttactgatttgtgctcaaattttactctttgtatgcctgttaactaccaagttgtctgaaaatgtgtactggttttatgctaactagtaaacaaattttactatcaaattttactctttatatgcctgttaacgagtaaacaaatttctcaggtatcattgtcactcgtgtttgacagaggggttaagagctgtgtaaggtacctggacgctgttccgtggagcgaatctgaagaggagacaatcaagaggctgatgtctcagcatccttcgtacaaggcagctttgagaaacctgctggcggcgaggctgcagcccgccgagaggcctgcctcgtcggcagaagcagaactggtcgttgaactggccgactccatcacgaaggggaccaacaacaacgcccggaaagagctgcggaaccgcatctgccattcgtgcctgaactctctggcgcgattgctgggagaagaatccccggagccggatcagacgtccttgccatcagttggaagaggaggagtaccggtaccgccacctgagactgagaccgagagcatctacaggctagtcgaggacatcaactggctgctgcagattctgatcgacaggcagatgggggaggagttcgtcgagctgtgggccgaccaaacgagcctcagcggcgtgcacggacgggcgtcgccgatggtgcgccacgagctgagccggatctcggccacggtcttcatcgccatggggagcggcaggctccagtgcgccggagccagacggttcggcgtcttccaggcctggttcaggccgatgctggcggacttcggttggctccggaggtaccccaagggcctgaacgtgacggccctggaggaagggatcgggcaggcgctgctgacgctcacgctggagcagcagcaggcgctgttcttggagtggttcggggctttcggcagccacggccgggagtgcccggacctgatgagagccttccaggtctggtggcggcggtcgttcgtgagatgagatcattaggtagccaaataaaaaaaaacagtaattctacagcagattggttgtttttatgggcggttggccattagtttccggtgggcccacattctcttatggcaatatgagttggaatgtcatcatcagtgatc

SEQ ID NO: 406 - DNA, 1327 bp, Zea mays
ctatctgtggccgagcgcaaccaagaaacgaggtatggccggagggagaggtagggctggtcggctggacctggtcggctggacgttgccggactggacttttccctgttggagagggggtagcgaggagggctgccgcaccgacgcgagcacgaggacgccgcggagggccgcgacccgaggccactggcgaggacggactgcagagcgctcgcagcggacgacccacgagcagagaagcgagctgcttacgaagctagatagaggatttggaacggcggtggccggcgaacaagcgggctgaaggagtgcggcttgtgggtcactcggcaaggctacgagcttcatcgaacaagaagacacgcgggtcaaggcaatcggcaggattgagaagacgtgtactgatcggccggagtggaagaacaactcacatttgtgcagcttacccaagggatcttcagtgccgtcatattttgggaatccaagcttctgcgctcctggagggggtgggccgtgtattcacggtgtcgttgagagcgcaattgtactgtccaaggggctctgggtgatgacgggatcctctcctggcgctgagtcgttcaccgtgagaacaggccgaccggcggcagcagaagagaagatcaggcggaaggattgtctagtccaataccaaatgataggcacagggttacgagcagatttggtttggtaggtttcgagatgaagacgagggcaaggtctagcagtgccaactctaggtgtgctgctgcttacactgcgcggtggtggagctggtcatgccggcggccgtgcaagtgcccgccacgcccgtgtccaggaaggagacagggttttgacaggaaaggaagatgttcgtgcaataacaaaggaagctcgatctgtggtagaaccacacactaacaaaataaaaaggttggagtatgcgacgggtgggtggatataacttttacctgatgcgaggcaacaggtaaatgcttccgaggcaacaggcggatgctgaagcgtctttttttttcaatgatgcactttcacccgatcagaagcgtgtcatttctatgtgcttgtatcgtcttgatgagaaccattgggtttaataaagtctctttgtcgaaaaagaagcgtgccatttcacgttcggcctccactctcgtgtagggatgaatttgggtgtccggaaatttaaattattcgtattcatattcattaaaaatgcaaatatagatatctgtatgggtatttgattttaatattaaagttttcagatttgattcttgctgagtacatatgtgatgggtccgttggcttt

SEQ ID NO: 407 - DNA, 460 bp, Zea mays
cggcggcgtgactgactgctacatgtcgtcatcggtaaaggcgattataccttgtttccgagtccattggttctttagtctagtcgtgagttgtgcctgtgacagcacaaaaggagtgagtgttatgcttcgacatacagtattacttagacgtgcgtagagacgagcattttgaaaagaaaaaaaaaagtgtagaggtctcaagggtttcgaaagcgggcatttcaaaagattttgcgctggctctaaaaaaagatctgttttagcataaccgagtgaaatcagagtgtcagacgcgagcgcacagatttttcaggaagcagctggcaagagcttcgtcaagcatgtattaacagaaaacataatttgtataaaaaaatagcgcacaaatttgattgtattggatttcgctgggatcttgttgattgcataaattatttggtcttttacgagcactccg

SEQ ID NO: 408 - DNA, 1662 bp, Zea mays
actggacgcctgcagtaccggtccggattcttcagacgagggacgcgtccggcagcctttacaatatcttcgagtacatggagcatgatcttgctggtcttgcggcaagttgttatgtcccgttcacgttgcctcagataaaatgttacatacagcagctgttgtcagggcttgagcattgtcatgacaataatgtattgcaccgggatatcaaggggtcaaacttgttgttggataataatgggattcttaagattgcggattttgggctggcaacattctttgatccacgacataaacgaccaatgacgagccgagtggtcacgctatggtaccgtccaccagaattgttacttggtgccactgagtatagtgtaggtgttgatttgtggagtgctggatgcattcttgcagagctcctgtatggaaagcccataatgcctgggcgcactgaggtggaacagttgcacaagattttcaagctatgtggttccccttctgaggagtactggaagaagtctaaattgccacacgcaacaatattcaaacctcaacaaccctataaacgttgcatcagggagacatttaaagattttcctacatcggctctgccattggttgaaactctgcttgcaattgatccagctgagcgacaaactgcctcagctgcattacacagtgattttttttcaaccgagccttatgcttgtaacccttcaagtctgccaacatatccaccaagcaaggagatggatgccaagttaagagacgaggaggctagaagactacgagcggctgcaaaggccaagggagaacagaaaaggactcgaccacgtgatcgatctcggagggctggaccagcaccagaagctaatgcagagattcaagcaaacttagaccagaggcgacgaatgataactcatgcaaatgcaaagagtaaaagtgagaagtttcctcctccgcatcaggatggaggtacgggtaatccgcttggatcctatcatcatatggagcctatgttcgagcaccaggatgcttccttcagtacagtagtccccatagagaaggggacctcacaaacatggtccgggccgttgtttgatccagcagctctcgggcagtcgaggcggaagaagcaaactaccctagatgctaaggctgctgcttactcaaagcagctccaaaaggaaaagggagggatacgagcccgataacaaaatgtcgttttcctggtgagcatgtggagggaccgtgggaaaggttcatcgcctacggatgaattttcttgttttgttcctctggtggttggaaacaggcaacaatgcgctgctgtagggtgaagtcttccggattgtgtacatgtcatgtggtagcgtttccaagtactgtttagatgcttctgaacaagctgccgtcacccggacgggactttaggggtttgagactcgtatttgtgctctttatggcaattcagttgcccatgtgtatatatggagcaattctttcttgtgtatgtacagagacggcggtagctttgatttcttaataatgaatcaagtgtgatggcgaccttcctcacgtaaaaaaaaaaaaaaaaaaaaaaaaaaaaaaaaaaaaaaaaaaaaaaaaaaaaaaaaaaaaaaaacgaagcttacgcgtacc

SEQ ID NO: 409 - DNA, 756 bp, Zea mays
gagtagaaacttgttatcgaaaccttagtgaatcagctcgtgtttccatatctcttcctttgtcagttttttctggtaccgccctaacttttgtaaataagtgccgtcagatttgctgaactagcatcagcctaatagctgcattttgtttaaaaatattagtaattgtctagaaaatgagcttgtcaggtgcacttatatgcacctcagtacacgaaacccatccgaaggcttcatcgtttggcggacgctgccagtctctgtacacgcatagcgagcagctccctgataactatctccggttcgggcaggcgtttgcgaagtgggaaggatcgccgcagttaaagcagcagccgagcacggtgtcgcccgtcgctgaaacgaacatcccgccgtcagcgttacggcctctgcctctccggcctccgcctctccgtcctgcagatgcagacgcagacgcagacgcctgcctgctgctgccgcgcccacggcggcctctccccgtaccattgtccacttcgtcttcccatttgaagaagccacagccaggatcttgacatttgtagaacctcctccccctgttagcctctgtattagcaattcgtgatatgcaaagcgctccacatgaggtgcacagcacttgacctgcatctgatgcattacttggattttgggtatgggttgaacggaaaccttgagaatgcataaccgatgggttatcattattaagctggcttgccggaccaaaatagtatg

SEQ ID NO: 410 - DNA, 445 bp, Zea mays
  misc_feature (337)..(337): n is a, c, g, or t
ctcgatcacttctttcccttccacacaacacacagcttcctcaactcccctcccgcttcgtcagctcatcatcataaaccaaacagagagctctctctctctcggtcgttcaattccaggatgctgctcaacccggcgtccgtgctggacaccatccggcagcacctcctggaggagccaccggcggcggccgacgagagcttcgggatcctggtggcggaccagtggagcggctcgctcccgttccgcaccgacgacgccgatgacgatgacgaccccaccgatgaggctggccaggcggcgggtgacaggactttgctaggacgggaggaggagngacgtatcgttcagtgatctggaggacgacgaggacgatgataggagggagaagggattgaatgggcaagagccaaggtgggttggggaagcttgtacttttgttccg

SEQ ID NO: 411 - DNA, 654 bp, Zea mays
ccggccgggccgggcagtgattggtccaggaaagaacattgaagaaactgatattgggcagctgccctatctccaagccgtggtgaaagaaactttcaggctacatccctccgtcccgctgctaccgcggcgtgctccagtcgacacaaaagtaatggggtacaccatccctagaggctgtcgtatgttcataaacatatgggcagttggtcgagacaaagaaacatggcctgaacccgagacgtttatgcccgagaggtttttggggaagacagttgatctgagaggtggggattttgatctcatcccgttcggcggaggacgccggatctgccctgggatgccgctggcgattaggatggtgcatctgcttcttgcgtcactgttgaatcagttcacatggaggcttccagttgagattgaaggaagaggtgttggcatggatgaaaaatttggcctgactttgatcaaggctgtgcctctgtgtgctatggctacaccaatttgactttataatgttgtgaccgttaaatcgtgcatatgaccgtgaaaacactgttttgcacgagcattgttgatagtgtggaatttaaaataagatataagacgagtgagactttggaaaaaaaaaaaaaaaaaaaggcccctagggcc

SEQ ID NO: 412 - DNA, 991 bp, Zea mays
gctatcatctagagtagccccgttaaagcccaaacaaaaaccccgctgcgagatctgcgcatctcgctacaccaccaccacgacggccatggcggcgccgcgcgtcctcctcctcctcgccgccgcggcccttctcgccgtcgcctccctcggggacgcttcgggcgagggcccccgcgggcgcaagctgctggtgctcgtcgacgatctggccgtccgctcatcccactcggccttcttcggctcgctccaggcccgcgggctagatctggagttccgcctcgccgacgaccccaagctctcgctccaccgctacggtcagtacctctacgacggcctcgtgctcttcgccccatcgaccccgcgcttcggcggatcggtggaccagaatgctgttctggagtttatcgatgccgggcatgatatgattctggcagcagatcattcggcttcagatctgatccgcggcatcgcgacggagtgtggggttgactttgatgaggacccggaagcaatggttattgaccacatcaattatgcctccagtgaggttgaaggtgaccacaccttgattgctggcgatgacctgattcagtcagatgtgatattggggtccaaaaagattgaggctcctgtgctgtttcgagggattgggcatgcggccaatccatccaacagcttggttttaaaggttctatctgcctcgccatcagcgtattcagcaaacccggaggctaagtggcatctgttccatctctcactgggtcggccatatcgctggtttctgttatgcaggctaggaataatgctcgtgtgttgatatctggatcactggatttgtttagcaacaggttcctaaagtctggtgtgcagaaggctggcagcaaaatgagccatgacaaagctggaaatgaacaatttgtgacagagacgagcaaatgggtcttccatgagagggggcatctgaaggcagggaatgtcaagcaccata

SEQ ID NO: 413 - DNA, 703 bp, Zea mays
gcttccgcaggctccacacaagccaccggacacccgccgataccaccaccgctccctctccggccgccatgctgccgccggccgcccgcctcgcggcccgccgcctcctcggcctcgcctccgcgtccgcttcacagtccgccgcccgacgcctctccccgtctccgatctcctcccccgccgccgcggccgccacgggttacttctccaggtccttctcttcggcgcttaactaccacatcgattcgccggacaacaaccccaacatgccgtgggagttcaccaaggccaacatggacaaggtcaacgaaatcctatctcattacccaagcaactacaagcaatctggtattattccaatgcttgatcttgcacagcagcagcatggtggatgggtcccggttgcagcaatggacgccattgctaaaatagttggagttgcaccgatcagggtctatgaagttgcaacattttactcaatgttcaatcggaccaaggtgggaaaatatcaccttttggtgtgtggaactacaccctgtatgattcgtggttcacgtgatagcggatgtggctggttaagtcatcgtagagatgaacagcatgcggtgcctacggatggttgacttctctgcttgtgtaaatccagtgtaggaattggggttaaggtgatgtgcgtgaacgtggagagcaccatgaggaaagg

SEQ ID NO: 414 - DNA, 480 bp, Zea mays
  misc_feature (392)..(392): n is a, c, g, or t
cctgctccagctctagagacgtcgaagatgcgtcggagaagactgcacgcctgcctggagaagatgcctggagaagaccggcacgcctgcctgctctagctcgaggagcgctggagatccacccgcctggagaagaccggcaggccaccgtgtcgtcgaagaatcaggccgcggcgccggtgaagaagcaagtcgtgacgccggagatccgttcgtccgcccggtaggccgtcacgccgcatccgaagatctgccaggcgccggagatccgttcgtccgcggatccgtccgcccggtaggccgccgcgtcgcgtccatcgcaggcctccgcgtcgtcgaagaagcaggccgccacgccggtgaagaagcaaccaccccggtgaagaagcagnctgccgcgccgccgaggatgtgccaggcgaaggagagagggagagttggagtcgggatcggggaagaagaggctgttgcgcagaagat

SEQ ID NO: 415 - DNA, 612 bp, Zea mays
ttttttttttttttttttaacaaaaaaagaatctgatcatatatataaacatctctcagatacaatcacttcgggcgacgctgtgtgaaaacgactgcgattgttgcaactacaggatagctcctaaagccgttttacagtgcccccatcactcaaaatctaggggcatccacatgccgaagaaatgtctcaagctgaccatgaaccgttcgacaaaaaaaccctgccaattttgaccaatccccgcaccaccagctatgttcttcaaaggccaaaaaggggggaaaagaaagaatggatgtaggcgagtatgaacagttctgctgtgcaagtccaattgcgaaaatctcttcgaccgagcagatgaatggtaatggagccacctcgggccatggaatgactcggtcgaaggaacagaatggacttggtgggtagctacattagtatgcaaactcattgggctgtgcccacaaaaaaaatgttgtcttcaattcccagtttgccacccccccagtgatcttgctctgataaatgactgactgactgaagagcaggctggcacagagcttgtcttagtcgcccaccctctcgtaaaagaaatgtaggcctcgc

SEQ ID NO: 416 - DNA, 1199 bp, Zea mays
cttttttgggcccccccggttttcccatttttttttttccggtctgttttgtggggggatttgggggggaaaaaaatttccccggggaaaagcattttccccctgtttccgcccaggggccaaaataccccccacaaaaagggaaaaaaaagtgggggtccccccgggggggggggccccctttaaaaatagggcccccgggggggcaggaaggaattgccagacccaccttccctaacttcctaatctgaaaaaccaatgcagccataactccgccccgatctatccgtcgcccctccaaaagcacgagagcgcaggaagtgagagaataaaaataataataataaggggaaataataagaaaaaaattctcgtgttcttcgaccggtggtggtggtgcgaacgcaaggcgaccaaggggaggcgagatgaggggggcgaagcggccgctcagcgtggtgacgtcgtgggtgcggcggcagccgcccaaggtgaaggctttcttggccgttgtcaccggcatggccgcgctcgtcgtcatccgcttcatcgtccacgaccatgacaacctcttcgtcgctgccgaggctgcgcacgcgctcggcataggcgtcctcatctacaagctcacaaaggagaagacatgtgccggttcgtttccgcctgcttctttgccacccgttcccgatctgtcgagatcgcccgtgccggcgcagatagggtctaggatttttataggatttggcccgccaattcaacgtttcagacgacgattttactcctgtgaacatctaggactgggaaaggttagaatatttatttggtctattttgtatctataaataccacttatgtttttgtccaatttttagataatgtttgttgtgacctcagatgtgtagcaatgtcattgtttggggtcttgtacgggattcagtgctaatgtattcatctggtacattttggagcaaaaaaaaaaaaaaaaaaagcttatcgatacttcgtcccttgtgtgttagccacgacccacctcacagtcattgtgcccacgcgtccgaaacttttgcactcgtaaagtggattagcagagggccgttaattttttttttcagttgtaaactgtttacgtgaatgctgtaccgataccgtgataccgtctgactttttcatccatgctagagttgaagcaggtagcatcc

SEQ ID NO: 417 - DNA, 2662 bp, Zea mays
cctcgaggccggccgggacaataaaatacagctgcactggttagatggatgtgtttaagcctcttatgcactaggttgtgggttcgagcccccacaactacatatttttaaaaaaaaaaaaaaaaaaaaaaaaaaaaaaaaaaggccgggatagcgtcgtagtcggtactcggtagtcgtagtcaccgcgttcgcatcccgagcacgcaaccatggctcgactgtcagccaccgccgcgctcctcctcgtcgtcctctccctcctcaccgtcgcgcactgcggtgccctcgcagtcgcagcagagcacgacaccgagcccgaatcaaccgccgccgtggccgtcagcgacgacaacgtgctatccgaccccacggtccacgctgaggcgggcaccgccagcgccactgaagaggaggccatcccggcggagcagcagcagcatgggttcgaactcctccgcctgccgtctcaccggctccaccaccaccaccacctctggtggaagcgccaccacgggatctaccgtgacgcgtcgcacaagttcccttgttctggccacggcgcgaggcgcgagcaggcagtgagccaccttgccatcttgcccgcatccgcagaggtggccagggaggcgggggaggagatgcccgtggccgagccggacccggatcgctctctcccggacagcgacggagagggggaggtggctattacaagcgagcagccgtcgccgttcaccgacgccgacgggcacgacgacgagagctcggcggcagtcagggcgtggaagcacgagatgctgcggaggtggttccgcttccacttccaccacgacatgcgccgccaccgtctccaccacgtccaacacgaggaggaggaggaggaggctaccaccgccccagagacgacagggagcatgggcatgaagcggcaccggttccacttccaccaccacgacgacgaggacgaggagaagaatatggcggcgaggaagcggctcccgccacgctgatgagcacgacgaggacagcgacgacgaggaggatgagcaagtggaagagatggtccggcgcttcaggaaggcgatcatgaggaggaggttcggccacggccacggccatggacgccgttcccgcttcttccaccacatcgtccaccgccacgccgaggaggccaagaagcaggcagattttgaagaagaggagagcggtgtcctggcctggattaagggcctcatgaaccggttctagagctgcctcattcgagcgatccgtccactcacagtcacagccatggctgcttgcctttttactatttgccatcggccatcccgcgtgcatactgcagtgtcttggcacggctgcggccgcttggggatagcgtcgtagtccttatccatgttcgcttcccgagtatacgcacatgtatcacagattcacaagacgtacgctcctcctcctcgtcctctccctcctcgccgtcgcgcactgcggtgccctcgcaggcgcaggagagcacgacaccgagcccgaatcaaccaccgccccacagacgacagagagcatgggcatgaagcggcaccggctccaccaccaccaccaccacgacgaggacgaggagaagaatatggcggcgaggaagcggctccgcgcacgctgatgagcacgacgaggacagcgacgacgaggaggatgagcaaatggaagagatggtccggcggttcaggaaggcgatcatgaggaggagattcggccacggccacggccatggacgccgttcccgcttcttccaccacatcgtccaccgccacgccgaggaggccaagaagcaggcagatgttgaagaagaggagagcggtgtcctggcctggatcaagggcctcatgaaccggttctagagctgcctcattcgagcgatccgtccactcacagtcacagccatggctgcttgcctttttactatgagaacgtatcttttgctcatgggggcagcagcgctccttgctcttccaccaccacgacgacgaggacgaggagaagaatatggcggcgaggaagcggctccgccacgctgatgagcacgacgaggacagcgacgacgaggaggatgagcaagtggaagagatggtccggcgcttcaggaaggcgatcatgaggaggaggttcggccacggccacggccatggacgccgttcccgcttcttccaccacatccaccgccacgccgaggaggccaagaagcaggcagatgttgaagaagaggagagcggtgtcctggcctggatcaagggcctcatgaaccggttctagagctgcctcattcgagcgatccgtccactcagtcactcacagccatggctgcttgcctttttactatttgccatcggccatcccgcgtgcatactgcagtgtcttggcacggctgaatttacttggtgttattagtgtgttttctatccacggtgtgtcgtcttatatacgcacttgtatcacagattcacagtacgtactacctgcttgtgtgtgcttcacattgtgttgtttcatgtaattctgtgcagttatgtgccttttgttcttccaaaaaaaaaaaaaaaatgttcagtacgtactacccatacctagtgattttaattatttattgctaagagttcgaaaaaaaaaa

SEQ ID NO: 418 - DNA, 287 bp, Zea mays
cgagaccgtggcgtccgcacaaatcccgtgctcccccgtctgaaaaccctccccaacttcggcggcggcggcggcgctggcgaacgggtactctctccggcgagtctcccccctagtaaccaggaaatatggatgtacccggtgatgaacccgtggaggacctcacttgaagtcctaataaccctggcaatgagggagaagccactacttatggtaatgtgcaagattagttccagggaggaccaggaaagccttacgagggaccccaagaccccatgcaagacgag

SEQ ID NO: 419 - DNA, 446 bp, Zea mays
tatattgagcattgctagacacactgagacaatcgcacttgcaacaatctaacaacagcggctacctagagattagcctttcttgagcgcttcgccctttgtgagaacgcagcataagcggacataaaaacagactgctgactagctgctagagacgatattacacggtgactacgaccaagaaactatagtgggcttaggacaccgtgcttggcaagtctacatgcggcactgagagcgagccgtaagcggattacaacgcccgattggccgattgcaacgacaatacgagacacatctggcaagacaagccatagcatcgcggccggtataactgatactagcagcactgtgcgaccgtagcttgtggaaccgtaccgtctactatgcaaaatcatgtgcttgcagtcaacccactcgctattgctaacgggggagctatacca

SEQ ID NO: 420 - DNA, 865 bp, Zea mays
  misc_feature (819)..(819): n is a, c, g, or t
ttgggtacctcgaggccggccgggatccatcatgggctccaaatctccagaagaacatcagcaaggccacaatgggtcgtccggagacgccgcaattgctgtggttaacccgcctaaatcagccgtcgcgagcggcttgcttcaaggcatgctcgaagggcagggagaagacgatgaagacgacgacgatgagaagaatagagctgacgtgaagacaacggatggcgccaagaaaaagaggaagcgcaacaacaagaaaaacaacaagaaggggccagctacacaacagtcattcccgcctagggtccctctatcagatctattcgatggccgtccttacccggaaggtcagattgtcgactatgccccgaaggatgacaacctccaacgtaccacgaccgaagagcttcggcatgaagctgccatgagtaacatggacggagactttctcaaggactaccgcaaggctgctgagatacaccgtcaagtcaggcactatgcacaaaagctggccaagccaggtgtgtccatgacttaccttgccgaagagattgacgaaagtgtccgtgcactgactggtcaccatggtcttgagcctggcgatagtctcaaagctgggctggcatttccgacgggactttgcctgaaccatataggagcccactggacgccaaattcaggcgccaaagaagtcttccttcagcatgatgatgtcttgaaggttgacttcggagtccatgtcaatggcagaattgtcgatagtgcattcactgtagctgctaaccagtctacaacacctgctagcagctggtaaagcgngcaccacaccgnngctgaggaagcaggcatcgatgcccgaatcgac

SEQ ID NO: 421 - DNA, 740 bp, Zea mays
atctttctctctcgcgcgccatggccgagtaccacctcagcgcgcagctccacggccacgaggatgatgttcgtgggatctgtatctgtggtgatgtgggggtcgcaacatcgtcaagagataaaacggtgagattctggacgcagcacccggaaaagaagcgcgaatatgtcctctcaaaaacacttgtaggacattcaagttttgttggcccgatggtttgggctccaccttctgatcgctttccggaaggagcgattgtttctggtggcatggataccctagtattgctatggaatttgcatacaggagaagttgttggaacaatgaagggtcacacctcacaggtgactggtcttgctatcgacgacaacggtgacattatatcttcatcgatggattgtactttaagaaggtggagagatggcaatgctgtagaagtttgggaggctcataaagttgcagtgcaaactgttctaaagctgccctcgggagaactatttacgggttcaagtgattccaccattaaactttggaaaggaaggacttgtctacatacattctctgggcatgcagacactgttcgatgtttagcacagatgccagggatgggtatactttctgcatcacatgatggaactataaagttatgggcattaactggccaacccctgttggagatgattggccatacctctcttgtatattctgtggatgcgcattcatctggtctaattgct

SEQ ID NO: 422 - DNA, 1114 bp, Zea mays
cattgggtacctcgaggccggccgggagctaactttcttcgcgtatcagcctggcccgtgctcgcccactgcgctagggttcacttgcctctcgagttcactcgcagccgcagccgccgcggctctcgcctctccggttcactcgcagtcgccatcgcggaggcgttccagagcggcagccaccatcgccggcacctccacgtggtcgacctcgacgtgtcgtacgacttccagtggccctccttgatctagtccctgtcggccgccgccgcgagcacgagcgcgagaacgagcagcgcataacacgacggcgacggcgacgagccagtttccctgcgaatcaccggctccggcacgagccacgacgagctgcggcgaaccaaggcgcggctggcgcggctgctgcttccacctttcacggccagtctccggtacttctccgtggtgttcgactcgctgcacaagtgcctccccgccgacagcgccgagaggatcgccatcgagagcggccacctgggcgtggagatcagcgacgggttgggttttggtttgttaattagtttttttatgcttctttaatttcttctttgattgattacatatatagattgattgattacatatatagatatatacacccatgtaaagtagatgtcaaaccagttattgcacacgattgatacgaagtagtcccaagagtcgtttcaaaatttcacaatctaaattatcatttggagagtcatttttccataaaataccactatctatatattttcaatcttcaaaagtctttttatatcttatttatattttagagagtcaatcacgtttttatatcttatttatattttagagagccacatctgtatcaaattactctcatgagtttctgtttatgtttgttggtctagatactctcatgatttctaaggcaattttgtttctgtcacaaactagatatgcatgagaaactggatctggatatggaattacaaattgtttctggcagattctatttatgtttgttggtctgccaacaaattgtttatgttgctactatagttcatatgattgtaattcaaatgattggatattcacaaattgttggtctgcc

SEQ ID NO: 423 - DNA, 1141 bp, Zea mays
tatccttcttcctgcgctcgctattcatccaacactactccaagtttaatttcccacaccaaacctcagctcccaccgatcggttccatcaacctaacaatccctcgcttgttagcccgcgcgcgtgtgtcgtgtgcgctgcgtgcgtcccatggctcagcctcgcgtgcacccaaacgcggcggcggcggcgggtgacctggagatgggcggcaacggcggcgtgttcccgccgctgccagttcgcgggcagcccgcctccgtgaaccagcagcagctcggcggcggcccccacacctgggtgggcaacgacgccaacacgctgctggtggtggccacgctgatcaccacgctcacctaccagctcggcaccagcatccccggcggctactggcagcaggacacgacggcggcgtcgagggacggcaaggagcaggtcacgtaccgcgccggcgaccccatcatgcgcgacctgcaccggccaaggtactgggtgttcatgggcgcgagctgggtggggttcgcgagctccatgatgatgacgctgagcctgctggtgcggatgcccgttgactcgcgccacgtgcgctggtccttcgtggtggcctactccagcctggtgctcaccttcgtggtgtcgcagcccaagacgcacctgtcgctggacctcctcatctgggccgccgtcctcgtcttcctctggatcatcgtcagcctccacccggaccgccgcgcgcgcgtcatccaggagctctgctgcggtggccgcgagacctgaagcagagcaaaccccgaccgacgtaccacgaatgattagttgttgctttttttcttttctttttttggtgacgatggggtaattgactgtcttttctcttctagtcgaccaagatgtaaacaaaatatcccgctgcacaaggtagccagagcctatgtccttgattgagcgctatagtgctctacattcgtatcaagtagaatctagataggcagatgaacacgctggcatgatttagcgtaatcttttcggcacccgcctgcaatttaaaaaactagactagatatttccatgtcaacgtgtaaagaaaaaaaagaaaaatcaatggccattcgctagttttctttaaaaaaaaaaaaaaaa

SEQ ID NO: 424 - DNA, 2967 bp, Zea mays
gcttggcaaagagaccgataccacacagttagaggggccgctttttttttttttttttttcaaaaacagtagtaggataagactctgagcagctcagtagaacatttacataattacatggaacctcgcaccccacaaataacttagtcaagctcaaatgctactatgtaacctctattctagagcagttacaaatttaccaaccgattaacagtttagacactgatcacaagaaaaacaaaccatcctgaagcctaccagaaccgaataaaatgggaccagaatctctgagggcaacaagagcagaggctcaccaatttaccaaagattcacaaaatacatcatgttgctgatatcatcaaggatctcctgtgtgaagggctgaaggcaagaccaaggaatagaaaacattgacagtgatcccattgaggatagcagcagcagatcgcaacttttgaaatacccttatatccaagaaaaatggtgcaaaaggatggacaatactatgacctatggacctcccacaaataaattatctacaacaacaacaaaaggtaatgagctgattatcactcttgatcttccaaattttggatctgcaagagaatgtgttgcagcattaactcgaggatgtgtcaacctccttcccatgatcttcgtttattgttcttcgcagtcgtttcaaccagatctcatagtccatcgggtacggagttccacagaggctgtcgacataatccgcaaaagcttttaacactgctgagatgtcgcccaacttctgctgtatgactctcctagaccatgacgtctccctccactgaagtgcactttcgacagagtcaaggtcggggcaaactccgccacaagagaaatatagaaggtagattaaactttctgcgtcagacgctccacatagttttccttcctggagcgcaaatgtagacgagaagaatagattcattactggtcgatctctatcttctaggatagcatggccccatccaataagcacaaaatacggatgtgttgaaccattgttgacacagatcacattctccggctggatatcaccgtgacggactcctgctgatgctgctgcagcaagagcagataaacagtcatggcaacatctcagagcctcctcggcaccaaacaatccattccgtatcagattcgaaatggtctcaccaactggtgaggtgacaagcattggagtactgcaccatggatgaccacaacttgcatttgagttagggttgttacatgggcctggatggatgacccgtccggacgattccatgtgtggtatatatctgctagaaatgcatctttgcttcatcacggtcagtacttttgtctgcctctgtacttggtaccaggagtccatatcctcccatgccggatccaaatgagaagggctggagcctacatacagcaaacaagttttggtaggttcttccacaggagtagaagtataaataggaggatcactgtctgttaggatgtcactgatctgaaaacctttctgcatgtaagcatcatccatcaacacaacagatcctatttcaatccttaaggtttcatgtggtttcacatcgacagctgcttcctcctgtacctcaacaatttcaggagaacgactccgctgttggaatcgaaataaaccattaccattagcttgtctgtcaaccagatgtctttctgatcttcggctctgtagccttgaactatgttccgttgccagttcatcgagagaatacttaggctctccgtaaaattggatcatcgagtgaaatattgcgatgagaacctgaagggtcccaatgtctccccagttagcattcccaagtttgttccatactcggtctacggaacagactgaaattctggaatgtttctttatccattcagcagcacagtcataaacgttgttgagatcaaaatccagcctgttcaaatcaccttgcacttttaccactccgccaaactgtgaatcaaaaagaagcacgtaaacagagtccgaattgcgaagaacggtggaggacttgctgaggcttcgagatagcaggacacgcagagcaaagaacagtgcctcccctaagacagcaggagacggctcctcgtcaccctgtgcggaaaccattccgacatcaggtctcaacaaagccagcacgacgatatgatcccaccttccttgtgaaggcttcaaattcttcagcagcacagcagtcgcgcaaacaccatcgagccttccactgcttatagccttctcagcaggataaaatctggagctcgtgctgtgtacacaggcaaccgagaaaggcatcgcttcatcaggaccccagaaagtttgccacagtcctttaacgcctgcgtgaaggaaatcctccagagcaagatacgcgctggcctcaaaaacatccgacgtcgacgcgtacagagcgcttggcatccgtactagctgctgaaaagtaacgccctccagagcgtaatcgaaattgtgtaactctgtaagctcgaaaggaacgtcgaaacctggcttcctgaaaccacaatcttcgctgtgaccggagaaccagtcctctaggcttgacgtcagaagctcgctgtccacaaacttgcggagaaaatccttgcacgttgacgacgagaagttacggcgccggcctcgtgaggaagcgctatgctttttcggcgctgaagctgaaccgaccgatgtacggtcaggcgactggtcttccatgaggaggcggccgggtcgtcgttgtcgactggtcgtctcccccaggggccagggccgggccggcggctagattggagcggctggtggagctggagcggtagtcggacgactactcccgactgactccgtggtcgctggacggccgccccagccgcaacgagaagacatccgtcgccccccgcgaacagacggaaggagatggggaaaaggtggggtttttattt

SEQ ID NO: 425 - DNA, 1108 bp, Zea mays
aaaaaaaaaaaaaaaaaaacaaaaaaaaaagttctctttctcgcttcgatagctaatttcgccgtcacccgccggacagacacggaggtacagaggagcgagggggaaagtgggatggtagtgggggcatcatcggtgtccggcgccgcggcttccggaggtacctctggtacggctccgccgcggaaggggaagtcgtgcaagggctgcctttagtactcgtcagtgctcaagtcccgcggctacaacccaatctgcgttgggatcccccgttccatcccccaagttcctaactacgtggtggatgagcctaaggaggacgcgacagcgcaggggcatgatctcaggcggttcaggtacgggtgcgcgggttactccatattcgtgaacagggacggccaaggtggtgagggcgagggcaagacgttgttgccgtattgcaggggcctcgagttattggtggacagcagattggtcgagagaaaaccatccgcagctgaacaagctccagcacatgttacaaaggatgcagctaccacccgctctcaccaacaaggacaacagagacctgcgaacttgtcgaggcaggaattcttggagaggtttaagaagagtgctggactggtcgcttcaggggtcgcgaagaatctaaacaaaacggccaattatatcaaggaaaatattcatgacatattgtaccacgatcgtcgcccaccaaagtagtagccatgccgacttgctgaggtatatcatctatgctcaaggttgagagctgaagaaatgccttgctgctactggaacccttacacaagtactacctgaatcgccccctactcttgtattgtagtcccagaacgaagcacagcagccccagctccgtcactgattatgcaaatggagccttccaggattccaacttcactcactttacttgcataatcttgtaatttagggctgcatatgtacatgcaatttaacttgctgcttgtttgaagcggaaaatcagatttcgtttggtgtaatcggaagacgatgatattggttgaattttgactgaaaattcgagaaacagcatttctcgatgaaaatacacggtttctcggttgag

SEQ ID NO: 426 - DNA, 1014 bp, Zea mays
accagccgcctcatcctctagccgatctccttttttgtcgcctccgtgcgtgccccgggggccttgatcgcgcgcaccggtcagctcagcccgtctgattcgcgtgctgccagccctccgccgatcgtgaaacacaaccgtcggtggtttgcttgcttgatcgctgatcctcgcggacgctcacggtcatggcgcctcagcctttgcgcgctccgaggctgagcgcccccgctggccacgcggcgtgcaggtgctcggccgcgccgctattcgggaagcgggcgccccccgtcgtcgcgttcccccgcgcccgtagcggcggcgccgtcgcctcgtgctcggccgtgcaggagtcgtccacctccaccactgttagcaagaagaaggatgccgcggacggggccaaggaggccacagcggaggcggcgaagccggccgcggccgccaagccaaagaaggccgcggcgctgccgctgccggagatgatgcagcaggagatcatcccaccgctgaaggatgccttggaggcggaggacgacgtgtcccaggtgcagctcgccttccaaaacaacacgctggagggatccttcataaagaacgacgtcccttattatttttgggccttctttccaaaaggagacctcacaggaccgaagggctttgcgctatcgtcctacagcaacgaagtgagcaccatcgagccgttcctgatcgatgagaagaggataacggccaagtacgtggtgttctgggtctacaagaggctggctgggcaaggcatccttccggtctggaaggaagaggagggggaggaggaggccgcgaagtagtaggttgcgcaggcgcatacgcatacacatacacacacacagggtgctgccgtgaacatacacatgtaacgctgtaaagttcatctgctagtaaaagttcatctgctgtgaacatacacatgtaacacagatacttcagtttcagcagttaaaaacctaccgtcgttaaaaaaaaaaaaaaaaa

SEQ ID NO: 427 - DNA, 3667 bp, Zea mays
agtggacgcctgcggtccggttcggattctctggtcgacgagtgccatgcgcgcgctactgcatctgcggctcatcaatccgtgcagtcccagagcaccccgtcttcccgctactcccgcaagggcgctcgtcgcttccttctccggggcgtggcaggcaccttctggtagagcgagagcgagagcagcgccgccggcagttcgcgctatggcgtcccagcaggtgttccgggctcagcagcagcagcagcagcagttcccggctcagcagcaggagtcccagccggggaaggagcacgcgatggacccccggcccgaggccatcgtccaggactacaaggccgccaacaagctcaaggacaaggtggcgctcgtgaccggcggcgactccggcatcgggcgcgccgtgtgcctgtgcttcgcgaaggagggcgcgacggtggccttcaccttcgtgagggggcaggaggagaaggacgcggaggagacgctgcgtgcgctgcgcgacatcgggtccgagacgggcgcgcgcgagccgatggccctgcccgccgacctcgggtacgaggccaactgccgggaggtggtggagcgggtggcgtcggcgtacggcgggcgcatcgacgtggtggtgaacaacgcggcggagcagtacgagcgggagagcatcggggacgtgacggaggcggacctggagcgcgtgttccgcaccaacatcttctcctacttcctggtgtccaagcacgcggtgccgcgcatggagcccggcgcctgcatcatcaacacctcctccgtcaacgcgtacaagggcaacaagacgctgctggactacacggccaccaagggcgccatcgtggccttcacgcgcgcgctctcgctgcagctggccgacaggggcatccgcgtcaacggcgtcgcgccgggccccgtctggacgccgctcatcccggcgtccttcggcaaggagaaggtggagcagttcgggtcccaggtgcccatgaagcgcgccgcgcagccggccgagatcgcgcccagcttcgtcttcctcgccagcaaccaggattcgtcctacatgtctggccagatcctccacgtcaacggaggcgtcatcgtcaatagctagatacggaagctgttttgctgtcgtgtgatcgtgtccaagtccaagggaacaaagcgagtgtccgtgtgtaccagtcagtaccaggtagcagtgtcagtacgcgcacgcaggaaggcaaggcagggttcagttgtagcggtgtgtaatgctgtcagcacagcacttgtaaagaaggaacgtgtctgaataaatatgcgtccaagcatgaagacttcgtacgtggtttgcacaaaataatcgtacgtcccggtgcagtggtactgctagtgcaccattctccccctctcttttggacgaggtttatcccggccgggacacgttcagtagaacactaaattcgaaaccaaggttcaaagtggtttttttttttttttttggcaacacattaaattcgcatatgcatttgagccaaatgaatcgcgtcgatcatggttacaacaggacgtatagcagtgtgcaacttcaacgttcaccacgctaatgagtcagcctttacctttatatattctaaaagcaaaatcacatgtcaacgcttatagcgctgatgttctctcgacactgtttagaaaacaaaaacaaaatatgcaaacaaaaaagacgaagctatcatcttcagccacggcgaagccatctgcttgcttgcgagtcaaaactggagacatcatcttaaagctccctcaaccttggttcttgctagcatcccgtatatggaacataagcttaacgcccacgtagaacgtagtaaatatgaaggcaagggaaggcagggcatccttcccagccattcttgatatcagaactttaatcccggatggccccacgacgtaaccaacaagatttgccaccataagacagctgattgtcactgcaccagcaattgctttaaattcacggtgaacaagcatcccaagggaactagttgcctgaaatttgctggacagagatttaaccaatatctctggaatgaaaaataagcaagtcaaccatgcccatgagataagtttccattctagatcatgccacactgctacaaacgtgaatatcacccagatactcagtagttttctttgagaaccaccaagaggaatgtataagtacctaaccaaccatttattaaaagaagcatgccagcttttccagaaactctccagatcatggcaattatttatacaccttggcatgttttcaggagtctcgactcctccaaccagtgaccaaaaccggaagtagcgccaaataagaaagaatttcaaccacataaagtttaacaccccataactgatgatgaaaacctcaaaaggggataactgctgccatagccggcttactacaaaggcattgtagtggaaaaagtgtgtcatgccttccataagaaggaaactcaaaatccaccttagaccataccaagatatttgtgcaaatgaataatttttctgaggtacctctaactgagcagcaaatgcattgtatccaacaataggtccagcaatatacactggagcatatgtcaagtagcataaatacataagaaatgtatatctgtcaacactgagccctctttcctgcaaagcaaagtagcatgtctttccagaataacaaacctgagatctttgcatatgtttcttgaagtcaaaatgagaagaatgaattgtccagcagtaatcacatccaaagctaatcatccgtaatacaacaaaattgaagcatatgtgccacctgaatgtaccccgatagttgtctagaaaagccatattttgaccgaacaatgagaatgaatatccttcataaactcggttaagaataagaacggacaagttgaagctccaaatgagacttgtgcaatacttataatgagcaaatagcttcactattgagtaattaatcagtgcaattaaaagaacaaaaacaacacaagcaccgtgaagatagcataaatagctcaaagacagtatgagccacaacaaagcagtacctcgacctttcaggccatagcaatatctcaacgtattgaccagaatcagaaatgctcccattacaacagtcagaattggcaagttcccgcggaagtttctccattgcgcatccgacaggtcattcagatgacccgcaagtgatcccacgcgtagtccgtagagcctcccggaatattcgtgcgagaggcgaagcgacctccagatcacgacgaggtagaagcccagcgcgtagagtacgagcagcgtaagttccacccggttccagggcacgccgcccatctccgccagacctcaacccccgacctcgctaggcgtgcgtgggaaattggggagagtgagcagcgtgggagcagggcccctgctcggcgagccggtggggaatgcgcggctgtgtccgggcgcagaggcagcaaggcgactgagatccgaggcggaatggcagctggcagtggcggacgatccaggcggaggcgacaccggcgactgcggtgagaatggaagcctagaaggcggcgaggtagaagagctgctggccgttggatgtggtatttttcgccggtttcccggccgg

SEQ ID NO: 428 - DNA, 1464 bp, Zea mays
atgggcggcggcggcggcaatccaaatctgatgatatctcaacctaggctagagtgcttgtgtctcgatacatggggcacccttgtgacacctcaaatcacctcacacaacaagctgcaccgcagcaacgaacacgaaccttggcagaacgaagctgaagtagttcgctgtgaacccagtatggcaaacgcgccgaggatcctggccctcggcctgctagcgctcctgtgcgctgccgccggcccggccgccgcgcagaactgcggctgccagccaaacttctgctgcagcaagttcggctactgcggcacgaccgacgcctactgcggcgacgggtgccagtcgggcccgtgccgctcgggcggcggcggcggcggcggcggcggcggaggcggcggaggcagtggcggtgcgaacgtggctaatgtggtcaccgacgcgttcttcaacggcatcaagaaccaggccgggagcgggtgcgagggcaagaacttctacacccggagcgcgttcctgagcgccgtcaacgcgtacccgggcttcgcccatggcgggacggaggtggagggcaagcgcgagatcgccgccttcttcgcgcacgtcacgcacgagaccggacatttctgctacatcagcgagatcaacaagagcaacgcctactgcgacgcgagcaacaggcagtggccgtgcgcggcggggcagaagtactacgggcgcggcccgctgcagatctcgtggaactacaactacgggcccgccgggagggacatcggcttcaacgggctcgccgaccccaacagggtggcgcaggacgccgtgatcgcgttcaagacggcgctctggttctggatgaacaacgtgcaccgtgtgatgccgcagggcttcggcgccaccatcagggccatcaacggcgccctcgagtgcaacgggaacaaccccgcccagatgaacgcgcgcgtcggctactacaagcagtactgccagcagctccgcgtcgacccagggcccaacctcacttgctaggcgacggcgtccacgttgtacccatggctcctgctagtgctagctcgtccacaataaatgctcggcgtcgcaccctcgcgaaaagtaaaccaagagtttactgactagtacgagctagctagcatgcagtaataataatcatcgtaataataataataaaacgactgggctgagccactttgttcgacctgaatgaataatgaagcaactgtatgttccctctgtacatagttgtgtatgcttgtgcttttactttccgtttgcagcatgggtctaacatcgaccgaatcaagaaatttcgagaataccaagagcccttaatcccgattggggatgggcgcaaaaaaaaaaaaaaaaaaaaaaataaaaaaaaaaaaaaaaaaaaaaaaaaagggcggccgctctagagtatccctcgaggggcccaagcttacgcgtacc

SEQ ID NO: 429 - DNA, 673 bp, Zea mays
gaacgcactctcctatatccgccgccatcgtctcgggctctcatctctaatcttcgggttcgcgtcgcagtgtcttccgcccgggctaccaccatgccgaagcagatccacgagatcaaggacttcctgctgactgcgcggcggaaggacgcacggtcggtgcggatcaagcgggccaaggacgccgtcaagttcaaggtgcgctgctccaggtacctctacaccctctgcgtccacgacgccgacaaggccaacaagctcaagcagtcgctcccgccaggtctgactgtccaggagatttaaggtatcaagccacaaactgtgctttttttgcggtgtcagggacgtttgctccagtgttactttagtttgtaggtgggctcatgaagtttgagtttgggatgcgaatgatgtgtcgaacctatgagcctgtgttgtaatggttaccccagaccgcaaaggatggcaatctatcataagttaaactgatttccgtgattttcgcttcccccccttttgtctgattcctttgttgtttttcgcctgagcctttttctgccacagcaattgcgaactttgacgtggcaatggtggaatgagttatgttgtatcatctttcttgcctgctcgtatttgtaatattgaacagatagcagctgaaaattttgtaggc

SEQ ID NO: 430 - DNA, 984 bp, Zea mays
cccacgcgtccgcggacgcgtggggttcgggtgcggcccatgtgagaaggcgcgcggtgtttgcttccgccgtcggcaaggaaggaaaaagagagcagagaaccctagctagagtttggcgagtggtgcggaggcgcggctgctcctgctcgtcgcgacgtcgaatttcagcgccgcccgcgcgatccttggtctcttcgcccgagaggcgcgacaatggctgggaaaggcgggccgaccaacctggagaaggagcagatgtttgggatggcggagaaggagatggagtacagggttgatcttttcaacaggcttacaaagacctgttttgagaagtgcgttgaaaaaaggtataaagaagctgagctcaatatgggtgagaacagttgcattgatcgatgtgtttcaaagtattggttatccgttattttaaaggtcttttaagctttgttcaaaacttagctccgttttaagtcatgtacgtttcagtttctatgcaaccttacagatcaatgacgaaacatgacacatatcttcgaaaaacattttctcctataaatagtctagatatggagtaaacagtttttttataagatcgcatctgttcggcgccattgatgcccagggctatgccccctaatgcaaactgtagatgcttaatgccgagacagaggtaagatttttgcttatcctcttaattgtgatgcaaactgttttgagaattttgaagatgttatccatctgttaagctaatgcaaacttcagttatcttagttttggtgctctcaagacgcagtggagaagatgaaaagtgcatgcgcctccatccgatgtattcgctcagtcgcttgtatattggattaagaagattgcattatgatgtaccgtgtattccgagtttgagacgttcctattatactttagaaaaaaaacaagagttacggttgtcctatgtattaaaaataatactttttgtatggcatt

SEQ ID NO: 431 - DNA, 1897 bp, Zea mays
gaaagctgggtacgcgtaagcttgggcccctcgagggatactctagagcggccgccgccgccgccggagaaatccatgtgtggtggagatctccatggtggtgggctccaccctgcgccgcgccagcgacggcagcgggaatgccctgggcggggccgcgtgcggggccacctcctcctcccccgcgctcgtcgtcgccgcagccggggcacgagcggtcggtggcggccgagccatcggtatcggagaagccgtccgagcctgtctccgagcggcggattctgtgcctgaatcatgctccatatgtggtccctgtccggccggtcgacgtcgccgccgtggcccgtctcctttggcatgacggtagccttctcggcgccagcagcggcggccttgccgttgccgccatcgccagcgccctgctcgacggactgctggcacgggaggagctgctgctcggtggtctccaacattgtcagtgccatgtccgtggacggcagcaagagcgagcagcaggtctcacgtatatggtaacggaaatatgacgacgtgagtttgagatcatcgttacctgttaatggagttgacatgtccattttagagtattgactatgcccaacttttttataatagagaaatcctcgcctgagtctctatttcgttgaagccacatccgtcctcccgcccgcgcctccgtggtcgacctccagctcgcagtgcgcccggggtcgatccagcccacatctactccctccgaccctcgtcgcctccgcgctctccagcgatgacttcccgcgcctccatacctcgcaccagctcttttctctggccacttctcgcttgaaccgcctccgccgccccgacctcgccgcctcgctcctcgacctcctccttgcctccgccccgccctccccggggctccttgcgcgggcgctctccctctacccgggacccgacgatgcgctccgggccttctcctcgtctgcccccgccacccgctcccctcctccgcaccttctccgcgctacccggatacagtggttctccaagaaaggcgagacagccaagggggaggagctgcttgatgtgatggaatcaaaggaggtagccccgaattaccttacctacaatgctatcatgcaggggtattgcaaggaggggaatgtcggcgcagcgatgcgtgtgttcaagaagatgaaggtgatgaagaggcgggaggggagaagtgacttgggtgtgtctgctcattcgcagatatatgtggtgctgtttaggagtttggtggagaaacagaagtttggtgatgcgctgtggatttttaagagctgctttgcaatgaaggcagcaccaccatttgaggctgttaaaggtttggtagagggtttggtgaaggcaggcaggtcagcagaggccaaggatgttgttgccaagatggcctttcttgtgaaaggcgatgctaaacttgcttgggagaaggttgttggcgaattttttttttcaaagagggaacaacaaatttgaatccatgatttgctaggctttcatgcattttggacttggaatgatgatggcaaagaagataggctttcaacatgggatccaaagttgccttcttgttgtcactggagtgtattttgcaaggtccgtggttagatcaggatgtaatggatgctccctttactgtaagaacttgctgaattttgtcttttaataattttgaaatgatgttaaagtttatatttgttgaagtgcttaggagatcatgatagatgatacatatactatctatctatcatcttaacatgtttgttgtaatgtgtgtttgatgcctcatcacataattgctctgcttttcctttgaaaaaagggaaatttggattcaaaaaaaaaaaaaaaa

SEQ ID NO: 432 - DNA, 2796 bp, Zea mays
acgaagtaatatttggtcctggagaggctgaccaatgctcgcagtggggtttgaggaagagtggaaacatattacaacagcttcctgcaagcaggcaaagcatgttgttctctgcgacgatgcctagctgggtgaagaaactgtccagacggtacttgaacaaccctttgacgattgacttggttggcgatcaagatgaaaagcttgctgaaggaatcaaactctatgctatcccactcacagcaacatcaaagcgtaccgttcttagtgatctaatcacggtttttgcaaagggtgggaaaaccatagttttcactaaaagctggaactccgccgcggtggcgggcggctctaaaaataaacggatcccccccccctccccgagactgggagagaagagagatcactaactccccggcccccccctctagcacctaccagagagttagccaaacaagttgagaaggaaataaaggaatcagcacccaagcttggcacggtgtgtgtttatggcggtgtctcatataatgtccagcagaatgcactctcgcgtggtgttgatgttgttgttggcacacctgggcgcataattgatttgataaatggtggaagcctccagttgggagaagttcagtatttagtcctggatgaagctgaccaaatgcttgcagtggggtttgaggaagatgtggaaacaatattacaacagcttcctgcggacaggcaaagcatgctgttctctgcgacgatgcctagctgggtgaagaaactatccaggcggtacttgaataatcctttgacgattgacttggttggagatcaagatgaaaaacttgctgaaggaatcaaacttcatgctatcccactcacagcgacgtctaagcgcaccattcttagtgatctaattacggtttatgcaaagggtgggaaaaccattgttttcactcgcacaaagaaagatgcagatgaggtatcattggcactgacaaccagtattgcttctgaggcacttcatggtgatatttcacagcatcagcgtgaaaggacactaaatggatttcgtcaagggaaatttactgttctagtagctacagatgttgctgcccgcggtcttgatataccaaatgttgatttgattatccattacgaattgccaaatgatccagaaacttttgtccatcgttctggacgcactggacgtgctgggaaagcaggaactgcaatcttgatgtttaccagcagccaaaaaaggacagttaagtcacttgagcgtgatgttggttgcaagtttgagtttataagccctccgtccatagaagaagtgctggagtcatctgctgagcatgtcattgctacattgcgaggggtgcacccagagtcaactcaatactttcttggagcagctgagaaattaacagaagaactaggacctcatgcccttgcttctgcactggcacatctgagtggattttctcagccaccttcttcacgttctctgatcagttatgagcagggatgggtgacattgcaactaactagagaaccgggatatggcagaggcttcttttctcctagatctgttactggttttctatctgatgtttgttcagctgctgcagatgaagttggcaaaatatacataacagcagatgagaatgtccaaggagcagtcttcgatttacctgaggaaattgcaaaggacctgcttactatggaggtccccccgggaaacacattaaccaaaatttcgaagctgcctgcgttgcaagatgatagtcctgctactgatagctatggtcgcttttcaaacgaccgaggttccaggaacaggcgttccaggggtggaggcgcttcaagaggccggggtggttgggacaccgacagtgaggaccgataccggcgtggtggcaggagcttgaggtctgacaacgatagttggtcagatgatgactggtcaggcggtgggagaaaatcaaaccgttcctcctcatcctttggtggccgctcatcatcctacggcagccgtggctcaccatcaccatccttcggtgtccgctcatcatccttgggcggtagggagagcagcagaagcttcagtggcgcttgtttcaattgcggggaatccggtccccgtgcatcggactgcccaaacaagtagatggcgcatttttccctgcccaagcaagtagacagctcgagcatcaggacgcacgcagagcgaaaacccaagcgcgcccctgatcgaccagtatactacgcctgacagccagctacagtttttgtgtggctgtctcgtcaggactgaagattttggcatgctgattccagttatttagatccagaggagtccttactctacagggatatgttgagtctcagtagaattgtgtaaccagtaaccacacacacgaacaatggcggaggactaaaatataaaatcatagctggcatattgaacacgtaacatatattgcagttggcatggctcttttcttttcttccttccaatttgactccagtggtcgcactgtgtcgatcgtttttacatgcactactacacatgcagcctcctttgcttgctggcagctatgcatagttgcgtgtgcgacacagcatctccaagctctggcaatcgcttgtcaaatgtctgctcctcagagctgtcctccgtatcaatcctctttctgacttttctagtcttctctagagattttatcttgtatatcttatttctcttcaacattttctaaatcatgtcctctatatgtaaatatctatattagaaacaatttcttc

SEQ ID NO: 433 - DNA, 1067 bp, Zea mays
  misc_feature (1013)..(1013): n is a, c, g, or t
cccacgcgtccgcccacgcgtccgaacccatgctcgagctccaccaaccaccccccgcggacagatcctcgcccttgccccctccccccatgacggccgcgtccgctgtcacgccccagcccccaaacccgtgcaccgtcgaccgctccccgcctatcccaccgacgcgtggtcccctgccccactgcagagacaccgtgatgcaggacagccgcgtcgtcccctcaaacccttcagagagtctacgtccccacgtttgcgggcaggcggagatctagatctgctctcctctgcttcgtcgctcggcggtctcgtcgtggcggaagaacaagcaacttgccatcgagtgaggccacacgttccgtccctacatcagtgtcgcggctccaatgagcgtgcgcgtcagtgacaccagcgggaggcgagggcaggcagtggtggcgatggcgtccgccgacgcgcgcttggcggggatcgcctcctccattcgcgtccatccctgacttccccaagccagaaggatgcaaccaaatgcgagatctacgtggccttcaacaccaatcacctgttaatggtcatcaggcttcgagagtgctctgggttctgataggatctctaaagtttacaccttgacataccctggaatctctaaccaatgttgcattccactttggtcctgtagttttggctgaccaacaagtaaatctggtacttcctagagcggttgcacggcaagcggctgctgctcatgggcgactcgatgaaccagaaccagttcgagtccatgctctgcgcgaggccttgccgaacaaggccagatgttcgagatgcacgagcacaggatcagcaagggccaaggcttcttcatcttcgagttcacggtaataataaatcagtcgagtgtggcattatgtaaagtagcatgggagctagagtaattattttttatctcagtatggttcccactcagggagctttggtgtttctgatctcaatgttttttggtacaaggnttggcatgcgtgcaagtgactttttccttgattatatgtgctgacttttggcac

SEQ ID NO: 434 - DNA, 1620 bp, Zea mays
tcacgctcgtggcccgtcgtgtcagtgtcagggccccacgaggcggcatcgcgtcgcgggccgccaggcgtgaccgaaacaaacgaacccttttgcctcggccgggtgccttgcccggcccatgccgtgcccggttgtgtaaatagaagataggcgacgaacacgaagcaagcaaggcagccgaatcccaactcctgtgtgtgcctgccaacgcctcgtcgcccgccgccgccgctgcttcgcccctctccgccagccgctcccgctcggagcacgccgccgccggcgtgggcgcgcaccgacggccgtcgtgtcgcatggatatggcccccgcggccgtctcctccaacccgcgctcggtcgaggaaatcttcaaagacttctccggccggcgcgcggggctcgtccgcgccctcacctccgatgtggacgatttctgcagcttctgcgacccagataaggagaacttgtgtctctacggccttcccaacggtagctgggaggtctcgccgccggcggacgaggttcctccggagttgcccgagccggcgctcggcatcaactttgcgcgcgatggcatgcagcgccgcgactggctctcactcgtcgcggtccactctgactcgtggctcatctcagtcgccttcttctacggcgcccgcctcaacgccaacgaccggaagcgcttattcagcatgatcagtgatcttccttctgtctttgaagcatttgcggacaggaaacacgtcagggataggtctggcgttgatagcagcggcaagtccaggcactcatcaaagagaggaaacgatggccatgcgaagaactccagggcagcagctcctgctgccaaagagtacgacgacgacgatgacgaggaggacgaggaacacacggaaaccttttgtggaagctgcggcggcctctacaacgcgaacgagttctggatcggctgcgacatctgcgagcggtggttccacggcaagtgcgtgcggatcacccctgccaaagcggaccacataaagcactacaagtgcccggactgcagctcgaagaaaataaggcagtaggtcccagtgtgcaccgccggccatcgctgacctgtctgatcccaaaccatactccattgcttgcctgccttgccccaaggtcgcagctagcagatgcgaattccttcatttgtggggggttgttgtgtgatggatagcctggtaggaagaggcaaaggactgatatataatgactcgtttattgttcatctttttgcctgcttcgtcggtcgtcgcagtcgttgtggctgcatgaactgatgagtggtttgttcgtgctagttgtaggctgtttcactgatttgcgtgcgtatgtggactggtcctgtcaaatttgtaggcctgaaaccgtacttccatttacgtggctacgcgagctgatttgccggtctgcctctcccatctgtatcctggacatggcagtatggtcacgaagtccagatggcgatatgcctgactgccggtaacggagggcagctgtggtgtagtatatctcagtcacatgggaggcgttagataatttttgaccaaatgttataaaataagacactattaaatagtttttttatat

SEQ ID NO: 435 - DNA, 1458 bp, Zea mays
cgggaagggaggggaggggaggggaggggaggggaggggagagaacgcgagaaactatgatgcgctgcgccgccgccgcgcttgcggtcgccctcatcgctgccgttgccgccgcaaccgctgcggggtcgccggccgcgggcacgtgcgcgcggaggggcgcgccgccgttcctcgacgccgtcggatcccgctgccccttcgtccggatcgacccgtccccgcctctcgagatcaatgaaccatctttacttttagccgctgctgggcatattttgatttgacaaagttccttgtttatatcgattaataaaatggaatttctgctggtgcctcgaatgtggatgggttatattgcgaatttggaaagttgtggtttcttatataccttgtcttctgctgaaggtgagaggagagactgttgatactgagttgaaccttcgaaggaggggtgcttcatactccattctcttttatgctgcatggtgtccattttctagcaaactgcgaccaatatttggaactctcagcactatgtaccctcagatacatcactttactgttgaagaatcttctgctacaccaagcctgttttcaagatatggagtccgtggtttcccagccattcttcttgtaaatgagactacgatggttcgatatcggggttcaaaagatcttaactctctggtcgatttctataaagaaactacaggtctcgatccaattgcatatattgatgttgttgaacaagagagcacgggaagcttgagctcagttatgccatgggatcgttctattcgtgaaatggccaaagatgagatgtatttgttagttgcagttctttttattatcgtgaaggttgtagcacacttcatacctgttgtcatgtctcatctgagagctttcttagttgttcgtgtccaaaacttgaacctagggatccgtaggggatcaaaccagctcctggaacgagccttgaatgttcttgatgtaaggaggctttggagcaagctgaggctgagcaataaggcaacagacttgaggaagggagcaagtaatgcccgagcttgggcttcctcatttccttctgtttcactgggtgaaccttcatcttcaagacaagcttagccatcttagagatgtttgttagggtagctgctcacatgtttccgtgcaaaaccatttaatctttgctccccactgtttctgtgcagtcaggaaggggggaaaaccttgcatgcatgtaaattttgaggaaatcatgtgctgttgtatattaggaatacctttgcatttgctggtgcgacgcctaatcttcctgtctattaaccagagtgtttatttggcaatgtatcatgaacttgtagggttttttttttagaatttctctcttgtttgtggtgctgttgtactttacactgtactcttcattaaagaacttggtttcacttcgttatt

SEQ ID NO: 436 - DNA, 3743 bp, Zea mays
  misc_feature (10)..(10): n is a, c, g, or t
gccggccggngatgcatagactccagttctcctctctccctctctctctcggtcttaaaaatccagcgccaacgcggccgccggacgcgcattgctctccctccccgcctcgtctcctcacttcattctcgccacatcttcctctgctcccctcctcccaggtggcgttcttggagctccagctcctcgagctgcgtctgtctacgcttgggtggcggcagcccgcaatggggaggagcagccgagcctgctcccttctcggttccgcggcccagctgctcctcctgctgctgctctcgctcggctccgcggccgcccagaagggctccacatggaagaccctgagcggcaaggctccagtaatcgttgctaagggcgggttctccggtctatttcctgattccagtgattttgcttatcggattcttgggaccattagctcccctgacacagtcatgtggtgtgatgttcggttgacaaaggatggcgatggtatctgcctaccgagcataaacatggataactgcacaatgatagacaatgtttttccagaagggaagaagacctacaatgttaatggtgtatctacggtgggatggttctccgtggactatacgagcactgatctgctgccaaacgtgacactgaagcaatcggtcctatctcgtacccctgtttatgatggttccatgttgataaattctgttgaaaatgttttcacctcatttaacgcctctgcagtttggttaaatgtccagcaagacagtttctacagccagtttaagcttagcatgcgaaactatatcctgtctttatcgaaacaattcattactgattacatctcatcgcctgaagtgaacttcctcaccagtatatctggaagagttagcaagaagacaaagcttgtgttccgctttcttgacgaaggctctattgagccatcgacaaaccagacatatgggtccatgttgaaaaatctaacatttgtcaagacttttgcctctggaatacttgttcccaaaagctatatctggcctgttacaccagataattacctactgccttatacttcagttgttgatgatgctcacaaagcggggctagaaatctatgctgctgattttgcaaatgactttactatcagctataactacagctttgatccattagcggaatatctttccttcattggtaacagtgccttctctgtcgatggtgtgttgactgatttcccgattactccttcagaggcagttggttgctttagtaacctgaacaacagcaagatcgaccatgctaaacctctggttatctctcataatggtgctagcggtgactacccagactgcaccgaccaagcttatgaaaaggcagttgctgatggtgcagatgtcattgactgtcctgttcaagtgaccaaagatggcatactgatatgcatgagttctgttgacctaatggatgtcactactgttggaaaatcatcatttacttcgcaagtaactaccatcaacgatctgaaggctggtcctggagtcttcaccttcaaccttacttgggatgatatttctaagaacctacagcccatgatatcgaacccaatgagcacctataaactgtacagaaatcccagaaacaagaatgcaggaaatttcatgagattatcagactttttgacgtttgcaaagggaaaggatttgtcaggaatcatgataactgttgagcatgctgcattcatggcagagaagcttggatttggagtggtagacgcagtggtcaaagcacttgatgactctggttatagcaaacagactgcccagaatgttatgattcagtcaaccaacagctcggtgctaaagaagttcaagcaggaaaccaagtacagccttgtctacatgattgaagaaggtgtcagagatgccgcaccttcatcccttgcagacattaagaagtttgctaatgccgtctctgttagcaccacatctgttttaccacagacccattattatttgacaaaccagaccaataagcttgtcacgtcccttcagtctgcgggcctccaagtttacgtctatgtgctcatgaatgagtttgcatctcagccgaatgacttcttcgcagacgccacttcacagattaatgcttatgtgcaaggtgctaaggtggacgggatcatcactgatttccctgggactgctcacagatacaaattgaactcctgcacgagcatgggaaacagcgcaccactatttatgcagcctccgcaaccaggtagcctccttttaaccatggccccagatgtacagccaccagcagcggcgccaatgccgctcttgacggacgctgacgttgcagaaccagccctacctccagtcagtaacaccacaacagctgcatctccttcgcatgccgccctcagaatgcgaaccgatgtctcgatcctcatcgcattgctgatgctatgtgcttccctcctcatctgaaggtagtcctgcacaacatcatatgcggcgtggtgcattagagctgaggatttcacattgagctgctaatagcaattctttccaccatttatgtagcgatttttttttcttgttctgtccggtgttttgaaggatggatggatgttcagcttgatacctcaacggtgacacttgtgtgtgctgccgctgcctctaggtatattttggtacatatatgtggtgatacattgttcaggtcatcttttctactgccaaaccctggcccggagggcaaaaatattcactgccatctgtgctaactgttgtcaggcatacctgctatcatcaattctttcccccccctaaaaaaagcaatttttttcttgttctgcccggtgttttggagaatggatggatgttcagcttgatacctcaacggtggcacttgagcgtgctgccagctgtctctaggtacattttggtacaaacgtgtggtgatacattgtccatgtccccgtttttactgccaacctgcctgtggagggcaaaatattccctccaaacatacagtttagtaaggctgtcagggtgttatataatttggtaaggctgtccccaatagatctacatatagtttgggcaaaaaaaaaaaaaaaaaaaaaaaaaaaaaaaaaaaaaaaaaaaaaaactttcaaccattctcccagatgcacagccaccagcagcagcgccaatgccgctcttgacggacgctgacgtcgcagaaccagcccttcctccggtcagcaacaccaccacggctgcatcgccttcgcatgccgccctcagaatgcgaacccatgtcttgagcatcgccacattgctgatgctatgtgcttccctcctcatctgaagatagtcctgctcaacatcatacgcggcgtggcgcattagagctgaggattttcacattgagctgctaatagcaattctttccaccacctttatgtagctatttttttcttgttctgtccggtgttttggaggatggatggatgttcagcttgatacctcaaaggtggcacttgagcgtgctgccagctgtctctaggtacattttggtacatacgtgtggtgatacattgtccatgtcaccgtttctactgccaacctgcctgtggagggcaaaatattccctacaaacatacagtttagggtctgtcttggatacacctcaatcctgtcagggtgttctatgatttggtaagaggctgtctccaatagatctacatatagttatgc

SEQ ID NO: 437 - DNA, 1421 bp, Zea mays
ccaagtggaactgatggacaaaccaatacaggttcgggctcgggttgatgatggaggtatcatgtggagtgagaatatagttccaaactgtaactgggagtctggagaaaccatgttttattctgaattcaaggtgctgctacttaagggaattacttctgacattgagtgggctggtgacgaccacctcatttacattacaacggagagtatacttcggccaggccttcaagcttccgaaagcaagagatatttatttgttaaatctggaagcaaaaatacaaggtttatattctacctggacatattaaaacaaaacaaggaacttgctgttttgacaccttgtgtgtatggcgttgatacaacacctagtcattgtggaaatcatgtctttatgaagagacggagtgatgaattgtataattctgaattggttgcttgtccactggataatgttgctgagatcacttctcctgaccactgctctagccctgagttgcaggacctgttgcatacccgacaacagatgttacaacaggtacagatgcatttgcagagagctcaagttcggatgaaacgccaggctggcaaaggaaggatggaacggtccttcagtgtgggtgatcgagtgtatgtgaagctccagccatactgccagtcatcagcagccgagagagtgaaccacaaactggcgtttaaattctttggccccttaagcatggtcaggaaggttaacccggtagcgtatgaagtcgctttacctgaaggaagcaatgtgcaccctgtctttcacgtatctcaattgaagtcatttgttcctgcaaatgttatttccagctcggttttaccagctctgactccagactaccagatccctgaagaggttatggactcacgactgcaacgaagggcaggcaaggaggtacctcaactgctggtacgttggactagttggccgccatctctggccatctgggaagatgagcactggatcaaggagcaatttccaagggcgccagcttggggacaagctgtctctgaaggaggaggagatgtgaacgacgctggtattcaggatggcatgggtaaagctactcaagggggcgcgggagaatcaccagcaggaggcgagggcgacgctgttccagcagctcaggcgcggcctgcggaccacaaggcccaacaagaggaggtgcacaggccttgagtgggtgaattagacctatgactatgtatcgtggctccactatatatgggttgttcgtagacagggagagggtgtttttttggctgtaagaactcaggcggcggcggcgatccgaggtgatcgtcttccgaatcggaccgtactttccacaccaccataatcaagcgtgtgctccgtcttaatcgaaaaaaaaaaaaaaaaaagcggcccggcc

SEQ ID NO: 438 - DNA, 1861 bp, Zea mays
cctcgaggggagacatatatgcaccacgcacgcgcgggcaccacagctagtagagcctgcctacctccccactgtcgtcattaattcaccaggctagagctagctctaccgccgttccccgatcgagcgtccgtcatggagattgatgagatggagagtggtgtcgtcaatggaagcggcggcggcggcggcgggtcgttccggcgggcgaggtgctgggagctgctgtgctcggcggcgggcatgctctggggcaaggtcgtcgggttcgcccgcaagctgggcaggatcgcgagggacgacccccggcgggtggcgcactccatcaaggtcggcctggcgctcacgctcgtgtccgtgctctactacgtcaggccgctcttcaacaactggggggtctccaccatgtgggccgtgctcaccgtcgtcgtcgtcatggagtacaccgtcggtggcacgctgagcaaaggcctgaacagagcgtgcgggacgctggccgccggcttcatcgccgtcggggctcacaaggtggcctacctgtgcggcgacaaggccgagcccgtgctgctcgccgtcttcgtcttcttgctctcgtcggcggcgacgttctcgcggttcatcccggaggtgaaggcgcggtacgactacggcgtgaccatcttcatcctcaccttcagcctggtggccgtgtccagctaccgcgtcgacgagctcatccgcctcgcccaccagcgcttctccaccatcgtcgtcggcgtcggcacatgcctctgcaccaccgtcttcgtcttcccggtctgggccggggaggacctccacaggctcgccatcggaaaccttaacaagctggccgagttctttgaaggactcgagtctgaatgcttcagagagaacgccacgttcgagaacttggaagcaaaacccttcctccaagtgtacaagagcgtcctcaattccaaggccaccgaagactctttgtgcaattttgccaagtgggagccctgccatggcaagttcaagttccggcacccatggagccagtaccagaagctcggcgctctctctcgccagtgcgcttcctcaatggaggctctcgcttcctacgtcatcaccctaacaagaaccgagtaccctgaagcgcggcccgagctacgctcggaggttcgaacagcgtgccgccagatgagcctgcactcggccaaggcgctccgcgagctctcggcggcgatgcggacgatggccgtgccgccgtccccggccaacgctcacatgtccgcggcagccaaagcggccaaggacctcagggtcgagctagaggacgcggacctggcgcaggcgatgcacgtcgctgttgtcgcgtctctgctctcggacctggtgaccaaggcgaagcagatcacggaatctgtcggtacccttgcgcgacttgcccgcttcgtcaagaacaacgaccatgagaacaacgctgacgacaaggatattgcggctatcgatgcagtgagctgagaaagcactagcagacggtcttgtggcggcaacaatatatcctaattaaccgtgcatccagacgttcatagctatctaattcgcacatatcgaaatcgaagatgggtacattttacatagggtacccttttacataaaaccacatcaggcgatatgcctgtatgcgaatcggatggctctgaacgtctagatgcatagttgcacagggtaatatgagtgcacaatatatatgtgttgccggcaatcttttcggtgggacttgactagtgatgaaatatttggcgtggtggggaatacatgtatcagaagcctttgctaaatgggataaaagtaagtcggaaaaaaaaaaaaaaaa